(19) United States
(12) Patent Application Publication
(10) Pub. No.: US 2024/0238424 A1
(43) Pub. Date: Jul. 18, 2024

(54) HETEROBIFUNCTIONAL COMPOUNDS AND METHODS OF TREATING DISEASE

(71) Applicant: Halda Therapeutics OpCo, Inc., New Haven, CT (US)

(72) Inventors: Samuel W. GERRITZ, Guilford, CT (US); Kyle J. EASTMAN, Killingworth, CT (US); Katherine J KAYSHER-BRICKER, Branford, CT (US); Taavi NEKLESA, Orange, CT (US); Kanak Shail RAINA, Branford, CT (US); Hao LI, Branford, CT (US); James John MOUSSEAU, Salem, CT (US); Nilesh K. ZAWARE, Hamden, CT (US)

(21) Appl. No.: 18/516,381

(22) Filed: Nov. 21, 2023

Related U.S. Application Data
(63) Continuation of application No. PCT/US22/31280, filed on May 27, 2022.
(60) Provisional application No. 63/193,822, filed on May 27, 2021.

(51) Int. Cl. A61K 47/55; A61K 47/60; A61P 35/00

(52) U.S. Cl. CPC A61K 47/55; A61K 47/60; A61P 35/00

(57) ABSTRACT

The invention provides heterobifunctional compounds, pharmaceutical compositions, and their use in protein degradation and treating disease, such as cancer.

### CROSS-REFERENCE TO RELATED APPLICATION

This application is a continuation of International Patent serial number PCT/US2022/031280, filed May 27, 2022, which claims priority to U.S. Provisional Patent Application Ser. No. 63/193,822, filed on May 27, 2021; the contents of each of which are hereby incorporated by reference in their entirety.

### FIELD OF THE INVENTION

The invention provides heterobifunctional compounds, pharmaceutical compositions, and their use in protein degradation and treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerase thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistant to currently available medicines.

The present invention addresses the foregoing needs and provides other related advantages.

### SUMMARY

The invention provides heterobifunctional compounds, pharmaceutical compositions, and their use in protein degradation and treating disease, such as cancer. In particular, one aspect of the invention provides a collection of heterobifunctional compounds, such as a compound represented by Formula I:

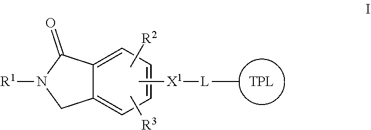

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a collection of compounds represented by Formula II:

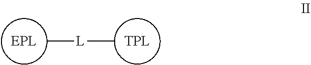

or a pharmaceutically acceptable salt thereof, where the variables are as defined in the detailed description. Further description of additional collections of related compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I or II, to treat the cancer.

Another aspect of the invention provides a method of treating hepatitis. The method comprises administering to a patient in need thereof a therapeutically effective amount of a compound described herein, such as a compound of Formula I or II, to treat the hepatitis.

Another aspect of the invention provides a method of causing death of a cancer cell. The method comprises contacting a cancer cell with an effective amount of a compound described herein, such as a compound of Formula I or II, to cause death of the cancer cell.

Another aspect of the invention provides a method of degrading a GSPT1 protein in a cell. The method comprises administering to the cell an effective amount of a compound described herein, such as a compound of Formula I, resulting in degradation of the GSPT1 protein in the cell.

Another aspect of the invention provides a method of degrading an effector protein in a cell. The method comprises administering to the cell an effective amount of a compound described herein, such as a compound of Formula I or II, resulting in degradation of the effector protein in the cell, wherein the effector protein is GSPT1, Cyclin K, RBM23, RBM39, IKZF1, IKZF3, a PLK1 degrader protein, a CDK4 degrader protein, or CK1alpha.

### DETAILED DESCRIPTION

The invention provides heterobifunctional compounds, pharmaceutical compositions, and their use in protein degradation and treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

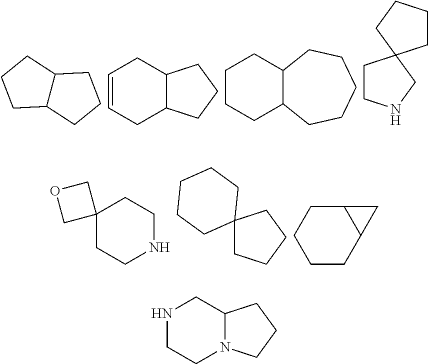

Exemplary bridged bicyclics include:

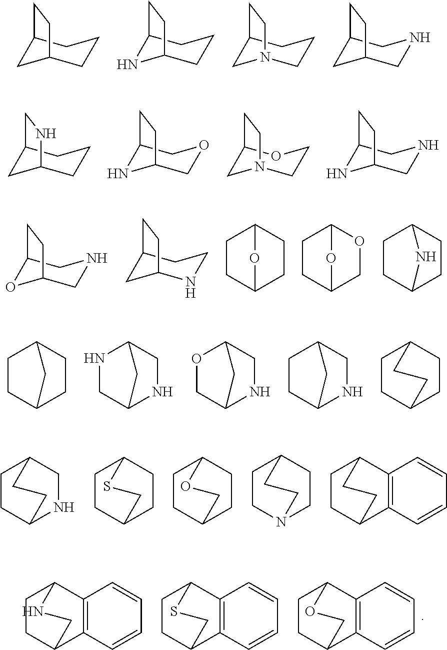

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene,” “imidazolylene,” and “pyrrolylene,” respectively refer to bivalent pyrazolyl, imidazolyl, and pyrrolyl groups. Similarly, the terms “pyridinylene” and “pyrimidinylene”, respectively refer to bivalent pyridinyl and pyrimidinyl groups.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄OR^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The term “amino” is art-recognized and refers to both unsubstituted and substituted amines, e.g., a moiety that may be represented by the general formulas:

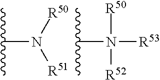

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

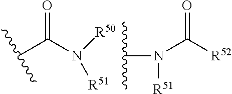

wherein R⁵⁰ and R⁵¹ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R⁵² is an alkyl, an alkenyl, or —(CH₂)ₘ—R⁶¹.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

The invention provides heterobifunctional compounds. The compounds are generally represented by the following formula:

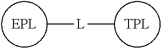

or a pharmaceutically acceptable salt thereof, wherein EPL is a moiety that binds to an effector protein selected from GSPT1, Cyclin K, RBM23, RBM39, IKZF1, IKZF3, a PLK1 degrader protein, a CDK4 degrader protein, or CK1alpha; L is a linker; and TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα.

The compounds may be used in the pharmaceutical compositions and therapeutic methods described herein. Exemplary compounds are described in the following sections, along with exemplary procedures for making the compounds.

### Part A: Compounds of Formula I

One aspect of the invention provides a compound represented by Formula I:

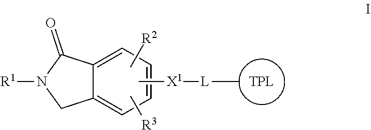

or a pharmaceutically acceptable salt thereof, wherein:
R¹ is

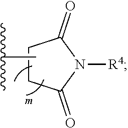

R² is hydrogen, halo, C₁₋₄ alkyl, or C₁₋₄ haloalkyl;
R³ is hydrogen, halo, C₁₋₄ alkyl, C₁₋₄ haloalkyl, or —(C₁₋₄ alkylene)-N(R⁵)C(O)N(R⁵)(C₁₋₄ alkyl substituted with 1, 2, or 3 substituents independently selected from the group consisting of halo, hydroxyl, —N(R⁵)(R⁶), —N(R⁵)C(O)(R⁷), —C(O)N(R⁵)(R⁶), C₁₋₄ alkoxyl, phenyl, C₃₋₇ cycloalkyl, a 3-7 membered saturated heterocyclyl containing 1, 2, or 3 heteroatoms independently selected from oxygen, nitrogen, and sulfur, and a 5-12 membered heteroaryl containing 1, 2, or 3 heteroatoms independently selected from oxygen, nitrogen, and sulfur, wherein the phenyl, cycloalkyl, heterocyclyl, and heteroaryl are optionally substituted with 1, 2, or 3 occurrences of R⁸);
R⁴ is hydrogen, C₁₋₄ alkyl, C₃₋₇ cycloalkyl, or —(C₁₋₄ alkylene)-C₃₋₇ cycloalkyl;
R⁵ and R⁶ each represent independently for each occurrence hydrogen, C₁₋₄ alkyl, C₃₋₇ cycloalkyl, or —(C₁₋₄ alkylene)-C₃₋₇ cycloalkyl; or an occurrence of R⁵ and R⁶ attached to same nitrogen atom are taken together with the nitrogen atom to which they are attached to form a 3-7 membered heterocyclic ring;
R⁷ represents independently for each occurrence C₁₋₄ alkyl, C₃₋₇ cycloalkyl, or —(C₁₋₄ alkylene)-C₃₋₇ cycloalkyl;
R⁸ represents independently for each occurrence halo, C₁₋₄ alkyl, C₁₋₄ haloalkyl, hydroxyl, C₁₋₄ alkoxyl, —N(R⁵)(R⁶), —N(R⁵)C(O)(R⁷), or —C(O)N(R⁵)(R⁶);
R⁹ represents independently for each occurrence halo, C₁₋₄ alkyl, C₁₋₄ haloalkyl, hydroxyl, or C₁₋₄ alkoxyl;
X¹ is

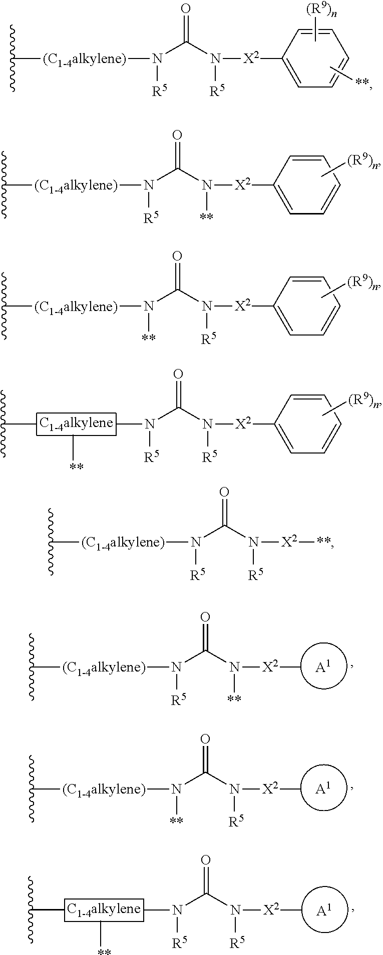

or a bond; wherein ** is a bond to L;
X² is a bond or a C₁₋₄ alkylene optionally substituted with 1, 2, or 3 occurrences of R⁸;
A¹ is a C₃₋₇ cycloalkyl, 3-7 membered saturated or partially saturated heterocyclyl containing 1, 2, or 3 heteroatoms independently selected from oxygen, nitrogen, and sulfur, or a 5-12 membered heteroaryl containing 1, 2, or 3 heteroatoms independently selected from oxygen, nitrogen, and sulfur, wherein each of the cycloalkyl, heterocyclyl, and heteroaryl is optionally substituted with 1, 2, or 3 occurrences of R⁹;
L is a linker;
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα;
m is 1, 2, or 3; and
n is 0, 1, 2, or 3.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, X¹ is

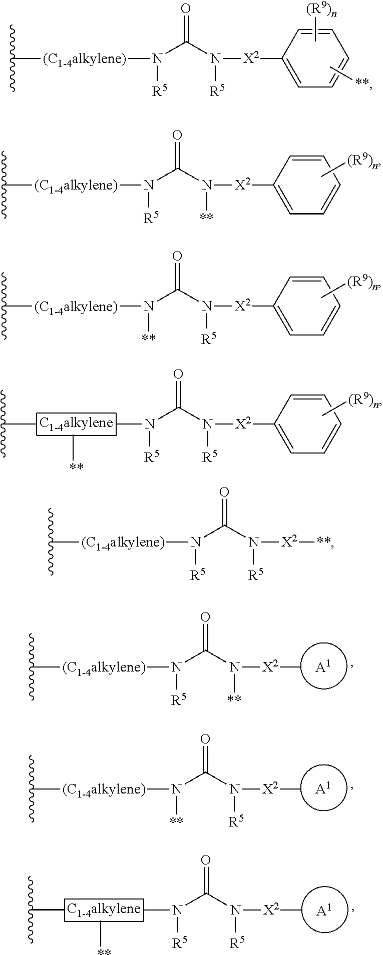

or a bond; wherein ** is a bond to L. In certain embodiments, X¹ is

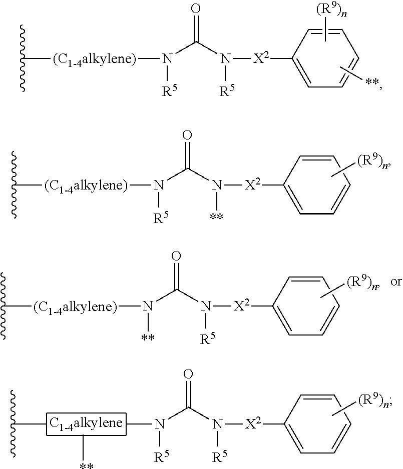

wherein ** is a bond to L. In certain embodiments, X¹ is

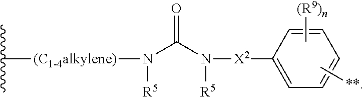

In certain embodiments, X¹ is

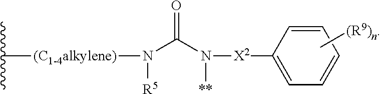

In certain embodiments, X¹ is

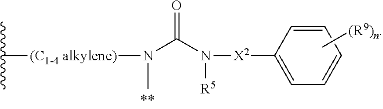

In certain embodiments, X¹ is

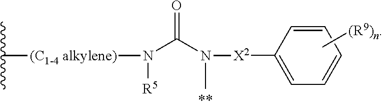

In certain embodiments, X¹ is

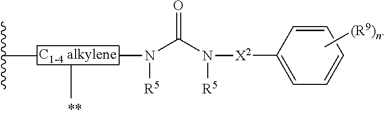

In certain embodiments, X¹ is

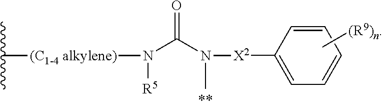

In certain embodiments, X¹ is

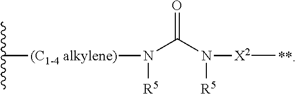

In certain embodiments, X¹ is

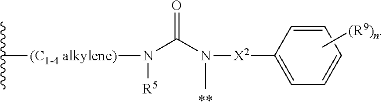

In certain embodiments, X¹ is

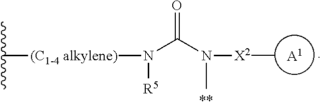

In certain embodiments, X¹ is

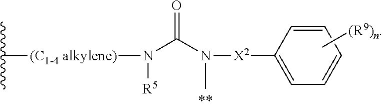

In certain embodiments, X¹ is

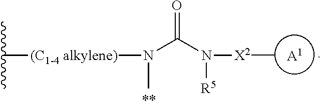

In certain embodiments, X¹ is

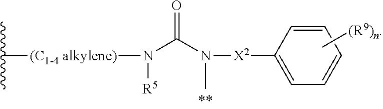

In certain embodiments, X¹ is

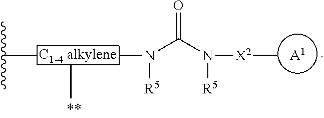

In certain embodiments, X¹ is

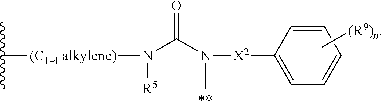

In certain embodiments, X¹ is a bond.

In certain embodiments, X¹ is

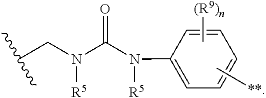

In certain embodiments, X¹ is

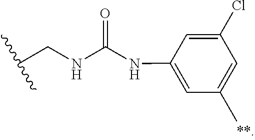 [SMILES: *CNC(=O)Nc1cc(C)cc(Cl)c1]

In certain embodiments, X¹ is selected from those depicted in the compounds in Table 3, below.

As defined generally above, X² is a bond or a C₁₋₄ alkylene optionally substituted with 1, 2, or 3 occurrences of R⁸. In certain embodiments, X² is a bond. In certain embodiments, X² is C₁₋₄ alkylene optionally substituted with 1, 2, or 3 occurrences of R⁸. In certain embodiments, X² is C₁₋₄ alkylene optionally substituted with 1, 2, or 3 occurrences of R⁸; and R⁸ represents independently for each occurrence halo or C₁₋₄ alkyl. In certain embodiments, X² is selected from those depicted in the compounds in Table 3, below.

As defined generally above, A¹ is a C₃₋₇ cycloalkyl, 3-7 membered saturated or partially saturated heterocyclyl containing 1, 2, or 3 heteroatoms independently selected from oxygen, nitrogen, and sulfur, or a 5-12 membered heteroaryl containing 1, 2, or 3 heteroatoms independently selected from oxygen, nitrogen, and sulfur, wherein each of the cycloalkyl, heterocyclyl, and heteroaryl is optionally substituted with 1, 2, or 3 occurrences of R⁹. In certain embodiments, A¹ is a C₃₋₇ cycloalkyl optionally substituted with 1, 2, or 3 occurrences of R⁹. In certain embodiments, A¹ is a 3-7 membered saturated or partially saturated heterocyclyl containing 1, 2, or 3 heteroatoms independently selected from oxygen, nitrogen, and sulfur, wherein the heterocyclyl is optionally substituted with 1, 2, or 3 occurrences of R⁹. In certain embodiments, A¹ is a 5-12 membered heteroaryl containing 1, 2, or 3 heteroatoms independently selected from oxygen, nitrogen, and sulfur, wherein the heteroaryl is optionally substituted with 1, 2, or 3 occurrences of R⁹. In certain embodiments, A¹ is selected from those depicted in the compounds in Table 3, below.

As defined generally above, R¹ is

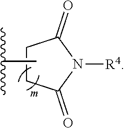

In certain embodiments, R¹ is

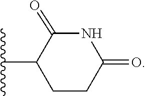 [SMILES: *C1CCC(=O)NC1=O]

In certain embodiments, R¹ is selected from those depicted in the compounds in Table 3, below.

As defined generally above, R² is hydrogen, halo, C₁₋₄ alkyl, or C₁₋₄ haloalkyl. In certain embodiments, R² is hydrogen. In certain embodiments, R² is halo. In certain embodiments, R² is C₁₋₄ alkyl. In certain embodiments, R² is C₁₋₄ haloalkyl. In certain embodiments, R² is selected from those depicted in the compounds in Table 3, below.

As defined generally above, R³ is hydrogen, halo, C₁₋₄ alkyl, C₁₋₄ haloalkyl, or —(C₁₋₄ alkylene)-N(R⁵)C(O)N(R⁵)(C₁₋₄ alkyl substituted with 1, 2, or 3 substituents independently selected from the group consisting of halo, hydroxyl, —N(R⁵)(R⁶), —N(R⁵)C(O)(R⁷), —C(O)N(R⁵)(R⁶), C₁₋₄ alkoxyl, phenyl, C₃₋₇ cycloalkyl, a 3-7 membered saturated heterocyclyl containing 1, 2, or 3 heteroatoms independently selected from oxygen, nitrogen, and sulfur, and a 5-12 membered heteroaryl containing 1, 2, or 3 heteroatoms independently selected from oxygen, nitrogen, and sulfur, wherein the phenyl, cycloalkyl, heterocyclyl, and heteroaryl are optionally substituted with 1, 2, or 3 occurrences of R⁸). In certain embodiments, R³ is halo, C₁₋₄ alkyl, or C₁₋₄ haloalkyl. In certain embodiments, R³ is —(C₁₋₄ alkylene)-N(R⁵)C(O)N(R⁵)(C₁₋₄ alkyl substituted with 1, 2, or 3 substituents independently selected from the group consisting of halo, hydroxyl, —N(R⁵)(R⁶), —N(R⁵)C(O)(R⁷), —C(O)N(R⁵)(R⁶), C₁₋₄ alkoxyl, phenyl, C₃₋₇ cycloalkyl, a 3-7 membered saturated heterocyclyl containing 1, 2, or 3 heteroatoms independently selected from oxygen, nitrogen, and sulfur, and a 5-12 membered heteroaryl containing 1, 2, or 3 heteroatoms independently selected from oxygen, nitrogen, and sulfur, wherein the phenyl, cycloalkyl, heterocyclyl, and heteroaryl are optionally substituted with 1, 2, or 3 occurrences of R⁸). In certain embodiments, R³ is hydrogen. In certain embodiments, R³ is selected from those depicted in the compounds in Table 3, below.

As defined generally above, R⁴ is hydrogen, C₁₋₄ alkyl, C₃₋₇ cycloalkyl, or —(C₁₋₄ alkylene)-C₃₋₇ cycloalkyl. In certain embodiments, R⁴ is hydrogen or C₁₋₄ alkyl. In certain embodiments, R⁴ is hydrogen. In certain embodiments, R⁴ is C₁₋₄ alkyl. In certain embodiments, R⁴ is C₃₋₇ cycloalkyl. In certain embodiments, R⁴ is —(C₁₋₄ alkylene)-C₃₋₇ cycloalkyl. In certain embodiments, R⁴ is selected from those depicted in the compounds in Table 3, below.

As defined generally above, R⁵ and R⁶ each represent independently for each occurrence hydrogen, C₁₋₄ alkyl, C₃₋₇ cycloalkyl, or —(C₁₋₄ alkylene)-C₃₋₇ cycloalkyl; or an occurrence of R⁵ and R⁶ attached to same nitrogen atom are taken together with the nitrogen atom to which they are attached to form a 3-7 membered heterocyclic ring. In certain embodiments, R⁵ and R⁶ each represent independently for each occurrence hydrogen or C₁₋₄ alkyl. In certain embodiments, R⁵ and R⁶ each represent independently for each occurrence hydrogen or C₁₋₂ alkyl. In certain embodiments, R⁵ and R⁶ each represent independently for each occurrence hydrogen. In certain embodiments, R⁵ and R⁶ each represent independently for each occurrence C₁₋₄ alkyl. In certain embodiments, R⁵ and R⁶ each represent independently for each occurrence C₃₋₇ cycloalkyl. In certain embodiments, R⁵ and R⁶ each represent independently for each occurrence —(C₁₋₄ alkylene)-C₃₋₇ cycloalkyl; or an occurrence of R⁵ and R⁶ attached to same nitrogen atom are taken together with the nitrogen atom to which they are attached to form a 3-7 membered heterocyclic ring. In certain embodiments, R⁵ and R⁶ are selected from those depicted in the compounds in Table 3, below.

As defined generally above, R⁷ represents independently for each occurrence C₁₋₄ alkyl, C₃₋₇ cycloalkyl, or —(C₁₋₄ alkylene)-C₃₋₇ cycloalkyl. In certain embodiments, R⁷ is C₁₋₄ alkyl. In certain embodiments, R⁷ is C₃₋₇ cycloalkyl. In certain embodiments, R⁷ is —(C₁₋₄ alkylene)-C₃₋₇ cycloalkyl. In certain embodiments, R⁷ is selected from those depicted in the compounds in Table 3, below.

As defined generally above, R⁸ represents independently for each occurrence halo, C₁₋₄ alkyl, C₁₋₄ haloalkyl, hydroxyl, C₁₋₄ alkoxyl, —N(R⁵)(R⁶), —N(R⁵)C(O)(R⁷), or —C(O)N(R⁵)(R⁶). In certain embodiments, R⁸ represents independently for each occurrence halo, C₁₋₄ alkyl, or C₁₋₄ haloalkyl. In certain embodiments, R⁸ represents independently for each occurrence halo. In certain embodiments, R⁸ represents independently for each occurrence C₁₋₄ alkyl. In certain embodiments, R⁸ represents independently for each occurrence C₁₋₄ haloalkyl. In certain embodiments, R⁸ represents independently for each occurrence hydroxyl. In certain embodiments, R⁸ represents independently for each occurrence C₁₋₄ alkoxyl. In certain embodiments, R⁸ represents independently for each occurrence —N(R⁵)(R⁶). In certain embodiments, R⁸ represents independently for each occurrence —N(R⁵)C(O)(R⁷). In certain embodiments, R⁸ represents independently for each occurrence —C(O)N(R⁵)(R⁶). In certain embodiments, R⁸ is selected from those depicted in the compounds in Table 3, below.

As defined generally above, R⁹ represents independently for each occurrence halo, C₁₋₄ alkyl, C₁₋₄ haloalkyl, hydroxyl, or C₁₋₄ alkoxyl. In certain embodiments, R⁹ represents independently for each occurrence halo, C₁₋₄ alkyl, or C₁₋₄ haloalkyl. In certain embodiments, R⁹ represents independently for each occurrence halo or C₁₋₄ alkyl. In certain embodiments, R⁹ represents independently for each occurrence chloro or fluoro. In certain embodiments, R⁹ represents independently for each occurrence halo. In certain embodiments, R⁹ represents independently for each occurrence C₁₋₄ alkyl. In certain embodiments, R⁹ represents independently for each occurrence C₁₋₄ haloalkyl. In certain embodiments, R⁹ represents independently for each occurrence hydroxyl. In certain embodiments, R⁹ represents independently for each occurrence C₁₋₄ alkoxyl. In certain embodiments, R⁹ is selected from those depicted in the compounds in Table 3, below.

As defined generally above, m is 1, 2, or 3. In certain embodiments, m is 1. In certain embodiments, m is 2. In certain embodiments, m is 3. In certain embodiments, m is selected from those depicted in the compounds in Table 3, below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from those depicted in the compounds in Table 3, below.

The compound may be further characterized according to, for example, the identity of L and/or TPL. Exemplary further embodiments for L and TPL are provided below.

Another aspect of the invention provides a compound represented by Formula I-A:

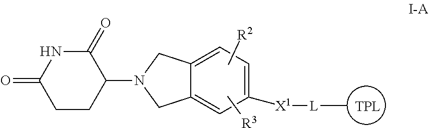

or a pharmaceutically acceptable salt thereof, wherein:
R² is hydrogen, halo, or C₁₋₄ alkyl;
R³ is hydrogen, halo, C₁₋₄ alkyl, or —N(R5)(R6);
R⁵ and R⁶ each represent independently for each occurrence hydrogen or C₁₋₄ alkyl;
R⁹ represents independently for each occurrence halo, C₁₋₄ alkyl, C₁₋₄ haloalkyl, hydroxyl, or C₁₋₄ alkoxyl;
X¹ is

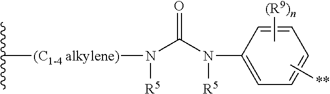

wherein ** is a bond to L;
L is a linker;
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα; and
n is 0, 1, 2, or 3.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

As defined generally above, R² is hydrogen, halo, or C₁₋₄ alkyl. In certain embodiments, R² is hydrogen. In certain embodiments, R² is halo. In certain embodiments, R² is C₁₋₄ alkyl. In certain embodiments, R² is selected from those depicted in the compounds in Table 3, below.

As defined generally above, R³ is hydrogen, halo, C₁₋₄ alkyl, or —N(R⁵)(R⁶). In certain embodiments, R³ is hydrogen. In certain embodiments, R³ is halo. In certain embodiments, R³ is C₁₋₄ alkyl. In certain embodiments, R³ is —N(R⁵)(R⁶). In certain embodiments, R³ is selected from those depicted in the compounds in Table 3, below.

As defined generally above, R⁵ and R⁶ each represent independently for each occurrence hydrogen or C₁₋₄ alkyl. In certain embodiments, R⁵ and R⁶ each represent independently for each occurrence hydrogen. In certain embodiments, R⁵ and R⁶ each represent independently for each occurrence C₁₋₄ alkyl. In certain embodiments, R⁵ and R⁶ each represent independently for each occurrence hydrogen or C₁₋₂ alkyl. In certain embodiments, R⁵ and R⁶ are selected from those depicted in the compounds in Table 3, below.

As defined generally above, R⁹ represents independently for each occurrence halo, C₁₋₄ alkyl, C₁₋₄ haloalkyl, hydroxyl, or C₁₋₄ alkoxyl. In certain embodiments, R⁹ represents independently for each occurrence halo or C₁₋₄ alkyl. In certain embodiments, R⁹ represents independently for each occurrence halo. In certain embodiments, R⁹ represents independently for each occurrence C₁₋₄ alkyl. In certain embodiments, R⁹ represents independently for each occurrence C₁₋₄ haloalkyl. In certain embodiments, R⁹ represents independently for each occurrence hydroxyl. In certain embodiments, R⁹ represents independently for each occurrence C₁₋₄ alkoxyl. In certain embodiments, R⁹ is selected from those depicted in the compounds in Table 3, below.

As defined generally above, X¹ is

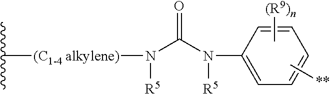

wherein ** is a bond to L. In certain embodiments, X¹ is

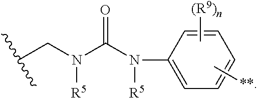

In certain embodiments, X¹ is

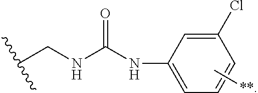

In certain embodiments, X¹ is selected from those depicted in the compounds in Table 3, below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from those depicted in the compounds in Table 3, below.

The compound may be further characterized according to, for example, the identity of L and/or TPL. Exemplary further embodiments for L and TPL are provided in Part C below.

### Part B: Compounds of Formula II

Another aspect of the invention provides a compound represented by Formula II:

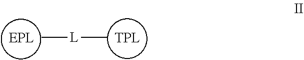

or a pharmaceutically acceptable salt thereof, wherein:
EPL is a moiety that binds to an effector protein selected from GSPT1, Cyclin K, RBM23, RBM39, IKZF1, IKZF3, a PLK1 degrader protein, a CDK4 degrader protein, or CK1alpha;
L is a linker; and
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

The compound may be further characterized according to, for example, the identity of L and/or TPL. Exemplary further embodiments for L and TPL are provided in Part C below.

As generally defined above, EPL is a moiety that binds to an effector protein selected from GSPT1, Cyclin K, RBM23, RBM39, IKZF1, IKZF3, a PLK1 degrader protein, a CDK4 degrader protein, or CK1alpha. In certain embodiments, the EPL is a moiety that binds to GSPT1. In certain embodiments, the EPL is a moiety that binds to Cyclin K. In certain embodiments, the EPL is a moiety that binds to RBM23 or RBM39. In certain embodiments, the EPL is a moiety that binds to RBM23. In certain embodiments, the EPL is a moiety that binds to RBM39. In certain embodiments, the EPL is a moiety that binds to IKZF1 or IKZF3. In certain embodiments, the EPL is a moiety that binds to IKZF1. In certain embodiments, the EPL is a moiety that binds to IKZF3. In certain embodiments, the EPL is a moiety that binds to a PLK1 degrader protein. In certain embodiments, the EPL is a moiety that binds to a CDK4 degrader protein. In certain embodiments, the EPL is a moiety that binds to CK1alpha.

In certain embodiments, the EPL is a moiety that binds to GSPT1. In certain embodiments, the EPL is one of the following:

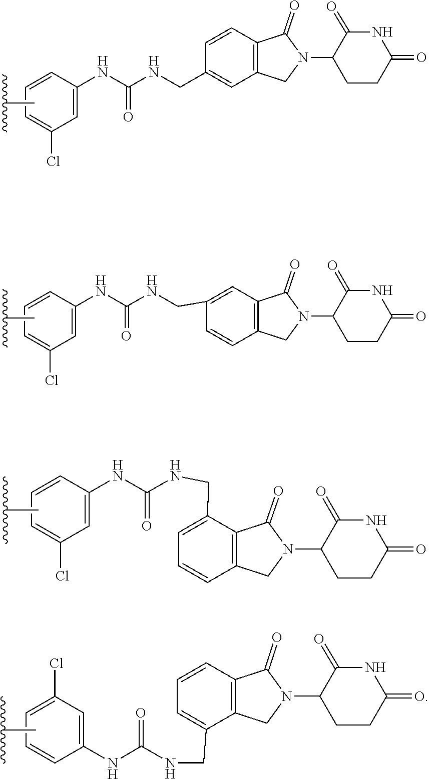

In certain embodiments, the EPL is one of the following:

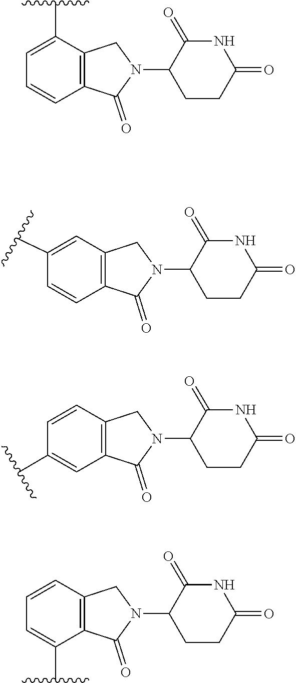 [SMILES: *c1cccc2c1CN(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *c1cccc2c1C(=O)N(C1CCC(=O)NC1=O)C2]

In certain embodiments, the EPL is one of the following:

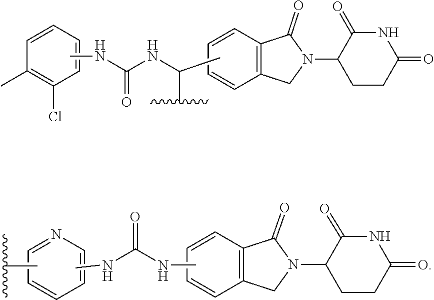

In certain embodiments, the EPL is one of the following:

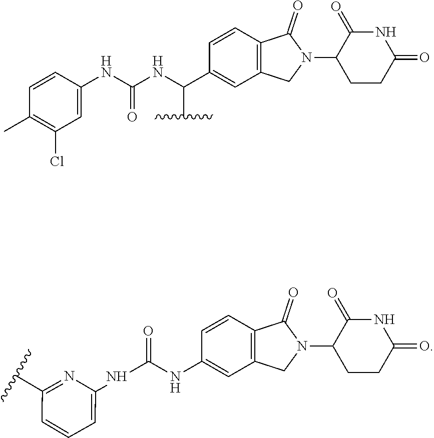 [SMILES: *C(NC(=O)Nc1ccc(C)c(Cl)c1)c1ccc2c(c1)CN(C1CCC(=O)NC1=O)C2=O ; *c1cccc(NC(=O)Nc2ccc3c(c2)CN(C2CCC(=O)NC2=O)C3=O)n1]

In certain embodiments, the EPL has the formula:

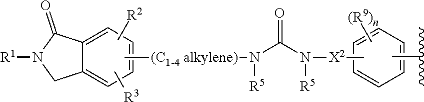

wherein:
R¹ is

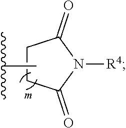

R² is hydrogen, halo, C₁₋₄ alkyl, or C₁₋₄ haloalkyl;
R³ is hydrogen, halo, C₁₋₄ alkyl, C₁₋₄ haloalkyl, or —(C₁₋₄ alkylene)-N(R⁵)C(O)N(R⁵)(C₁₋₄ alkyl substituted with 1, 2, or 3 substituents independently selected from the group consisting of halo, hydroxyl, —N(R⁵)(R⁶), —N(R⁵)C(O)(R⁷), —C(O)N(R⁵)(R⁶), C₁₋₄ alkoxyl, phenyl, C₃₋₇ cycloalkyl, a 3-7 membered saturated heterocyclyl containing 1, 2, or 3 heteroatoms independently selected from oxygen, nitrogen, and sulfur, and a 5-12 membered heteroaryl containing 1, 2, or 3 heteroatoms independently selected from oxygen, nitrogen, and sulfur, wherein the phenyl, cycloalkyl, heterocyclyl, and heteroaryl are optionally substituted with 1, 2, or 3 occurrences of R⁸);
R⁴ is hydrogen, C₁₋₄ alkyl, C₃₋₇ cycloalkyl, or —(C₁₋₄ alkylene)-C₃₋₇ cycloalkyl;
R⁵ and R⁶ each represent independently for each occurrence hydrogen, C₁₋₄ alkyl, C₃₋₇ cycloalkyl, or —(C₁₋₄ alkylene)-C₃₋₇ cycloalkyl; or an occurrence of R⁵ and R⁶ attached to same nitrogen atom are taken together with the nitrogen atom to which they are attached to form a 3-7 membered heterocyclic ring;
R⁷ represents independently for each occurrence C₁₋₄ alkyl, C₃₋₇ cycloalkyl, or —(C₁₋₄ alkylene)-C₃₋₇ cycloalkyl;
R⁸ represents independently for each occurrence halo, C₁₋₄ alkyl, C₁₋₄ haloalkyl, hydroxyl, C₁₋₄ alkoxyl, —N(R⁵)(R⁶), —N(R⁵)C(O)(R⁷), or —C(O)N(R⁵)(R⁶)
R⁹ represents independently for each occurrence halo, C₁₋₄ alkyl, C₁₋₄ haloalkyl, hydroxyl, or C₁₋₄ alkoxyl;
X² is a bond or a C₁₋₄ alkylene optionally substituted with 1, 2, or 3 occurrences of R⁸;
m is 1, 2, or 3; and
n is 0, 1, 2, or 3.

In certain embodiments, the EPL is a moiety that binds to an effector protein selected from Cyclin K, RBM23, or RBM39.

In certain embodiments, the EPL is one of the following:

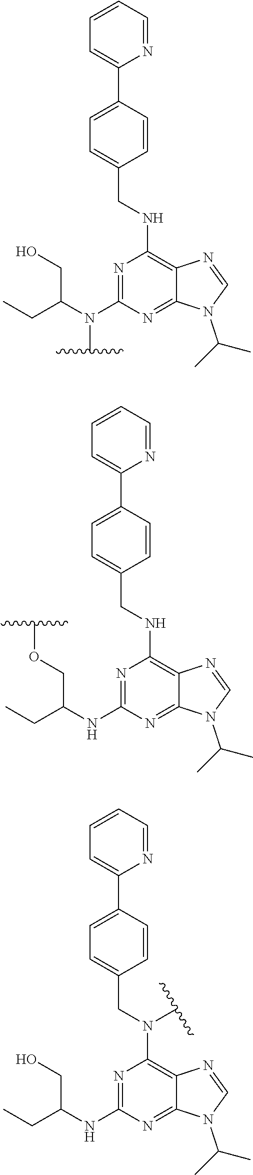

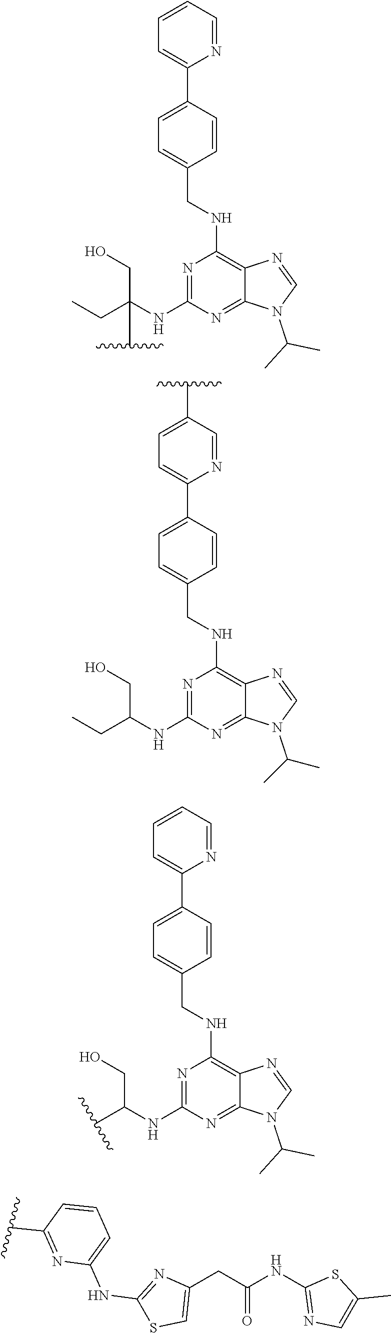

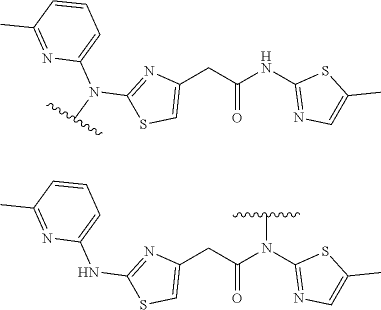

In certain embodiments, the EPL is one of the following:

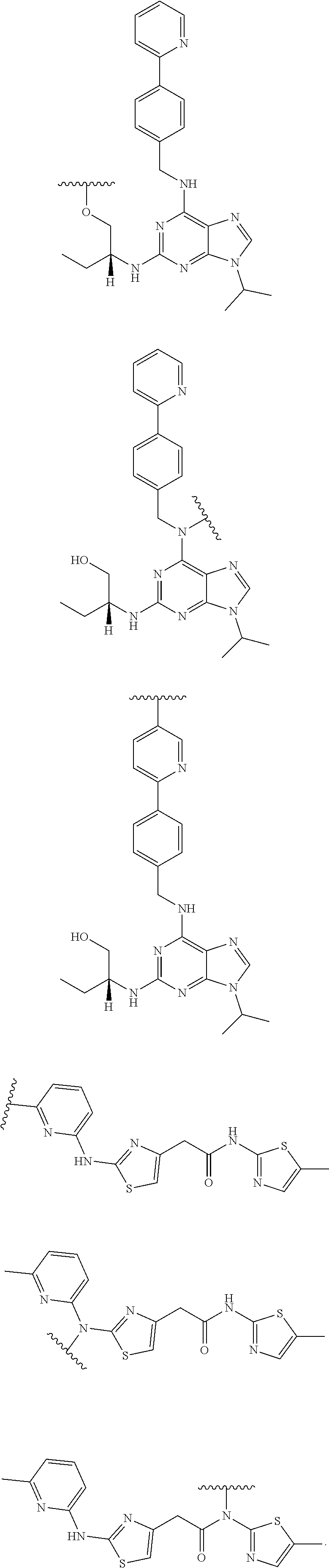

In certain embodiments, the EPL is one of the following:

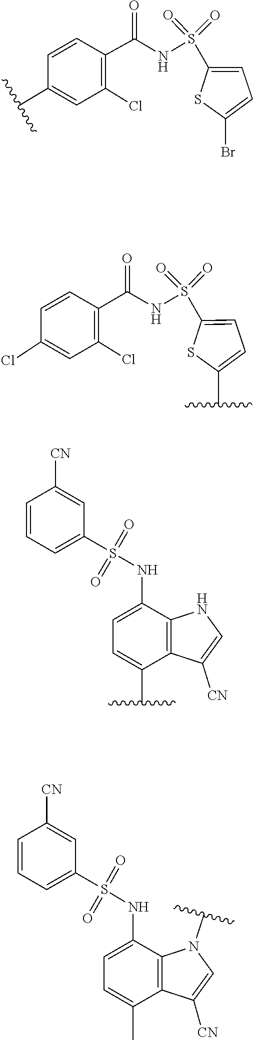 [SMILES: *c1ccc(C(=O)NS(=O)(=O)c2ccc(Br)s2)c(Cl)c1 ; *c1ccc(S(=O)(=O)NC(=O)c2ccc(Cl)cc2Cl)s1 ; *c1ccc(NS(=O)(=O)c2cccc(C#N)c2)c2[nH]cc(C#N)c12 ; *n1cc(C#N)c2c(C)ccc(NS(=O)(=O)c3cccc(C#N)c3)c21]

In certain embodiments, the EPL is one of the following:

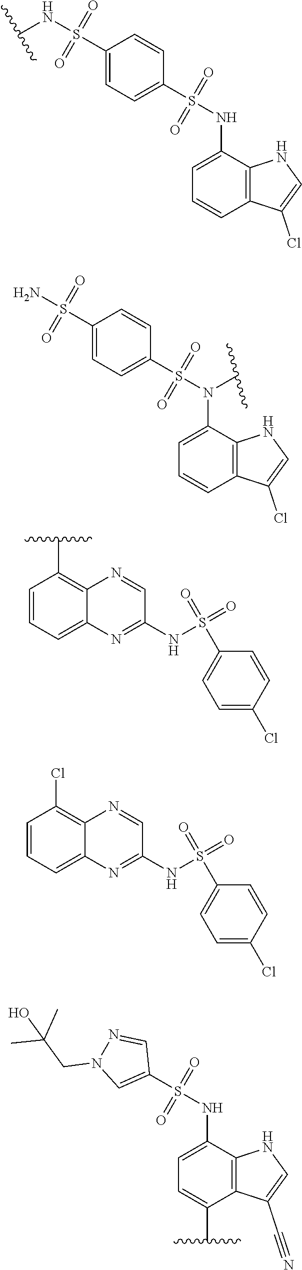 [SMILES: *NS(=O)(=O)c1ccc(S(=O)(=O)Nc2cccc3c(Cl)c[nH]c23)cc1 ; *N(c1cccc2c(Cl)c[nH]c12)S(=O)(=O)c1ccc(S(N)(=O)=O)cc1 ; *c1cccc2nc(NS(=O)(=O)c3ccc(Cl)cc3)cnc12 ; O=S(=O)(Nc1cnc2c(Cl)cccc2n1)c1ccc(Cl)cc1 ; *c1ccc(NS(=O)(=O)c2cnn(CC(C)(C)O)c2)c2[nH]cc(C#N)c12]

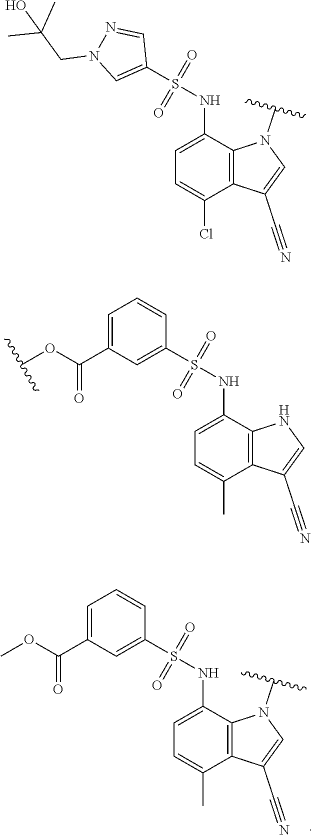 [SMILES: *n1cc(C#N)c2c(Cl)ccc(NS(=O)(=O)c3cnn(CC(C)(C)O)c3)c21 ; *OC(=O)c1cccc(S(=O)(=O)Nc2ccc(C)c3c(C#N)c[nH]c23)c1 ; *n1cc(C#N)c2c(C)ccc(NS(=O)(=O)c3cccc(C(=O)OC)c3)c21]

In certain embodiments, the EPL is a moiety that binds to an effector protein selected from IKZF1 or IKZF3.

In certain embodiments, the EPL is one of the following:

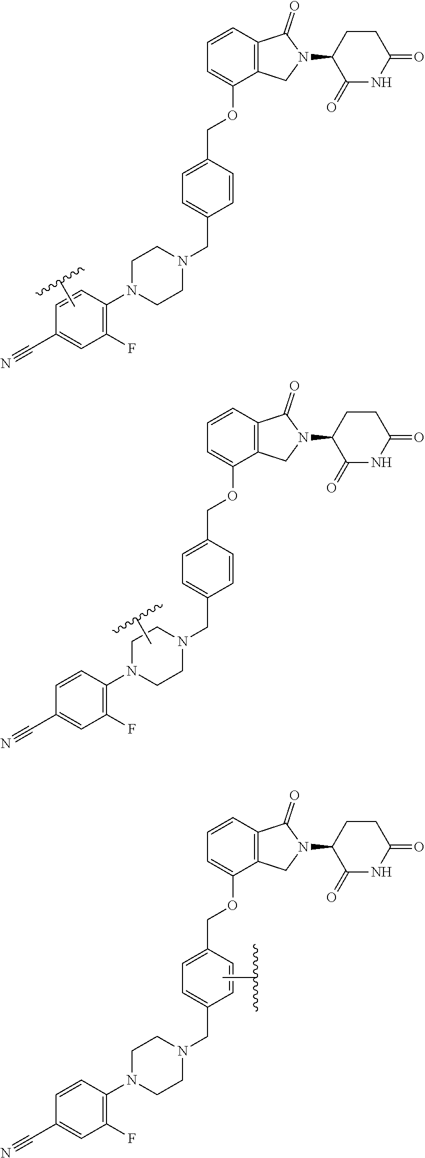

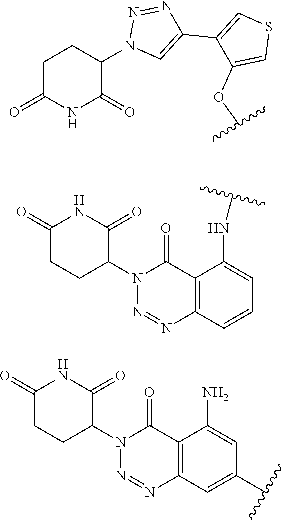 [SMILES: *Oc1cscc1-c1cn(C2CCC(=O)NC2=O)nn1 ; *Nc1cccc2nnn(C3CCC(=O)NC3=O)c(=O)c12 ; *c1cc(N)c2c(=O)n(C3CCC(=O)NC3=O)nnc2c1]

In certain embodiments, the EPL is one of the following:

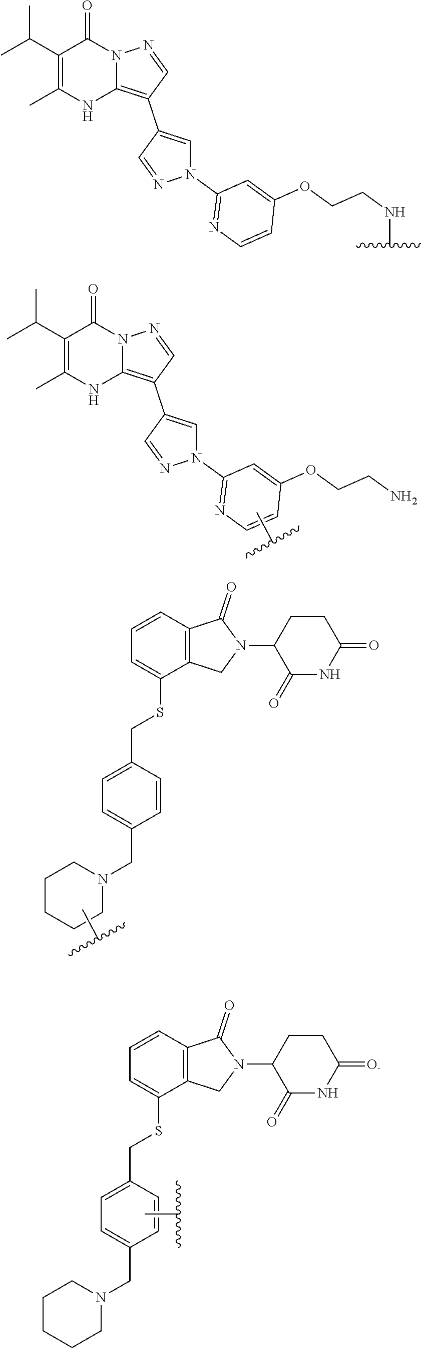

In certain embodiments, the EPL is a moiety that binds to an effector protein selected from a PLK1 degrader protein, a CDK4 degrader protein, or CK1alpha. In certain embodiments, the EPL is one of the following:

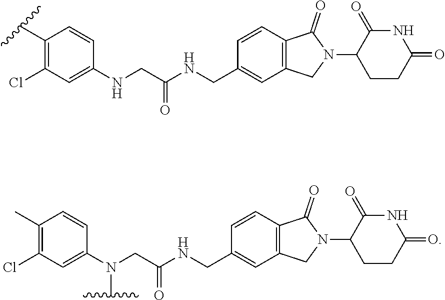 [SMILES: *c1ccc(NCC(=O)NCc2ccc3c(c2)CN(C2CCC(=O)NC2=O)C3=O)cc1Cl ; *N(CC(=O)NCc1ccc2c(c1)CN(C1CCC(=O)NC1=O)C2=O)c1ccc(C)c(Cl)c1]

In certain embodiments, the EPL is selected from those depicted in the compounds in Tables 3 and 3A, below.

Additional exemplary EPL components are described in more detail below.

### A. Moiety for GSPT1

In certain embodiments, the EPL is a moiety that binds to Eukaryotic Peptide Chain Release Factor GTP-Binding Subunit ERF3A (GSPT1). Exemplary moieties that bind GSPT1 are reported in the literature, including those shown below:

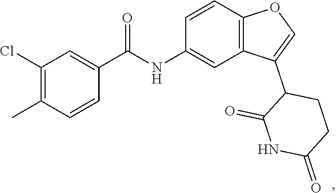 [SMILES: Cc1ccc(C(=O)Nc2ccc3occ(C4CCC(=O)NC4=O)c3c2)cc1Cl]

as described in Luo, Y. et al., in WO2021047627.

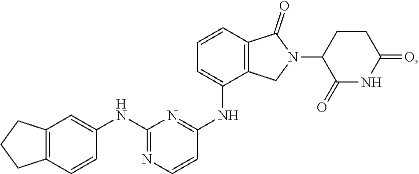 [SMILES: O=C1CCC(N2Cc3c(Nc4ccnc(Nc5ccc6c(c5)CCC6)n4)cccc3C2=O)C(=O)N1]

as described in Gray, N. et al., in WO2020006264.

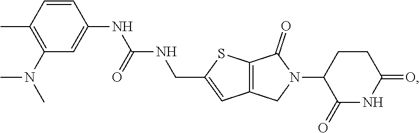 [SMILES: Cc1ccc(NC(=O)NCc2cc3c(s2)C(=O)N(C2CCC(=O)NC2=O)C3)cc1N(C)C]

as described in Chan, K. et al., in US2020369679.

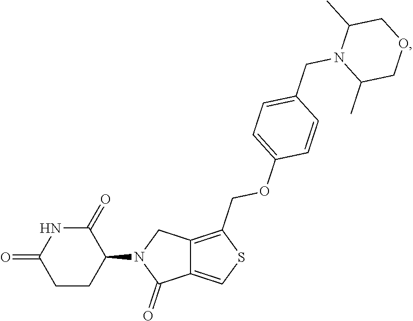 [SMILES: CC1COCC(C)N1Cc1ccc(OCc2scc3c2CN([C@H]2CCC(=O)NC2=O)C3=O)cc1]

as described in Chan, K. et al., in WO2019241271.

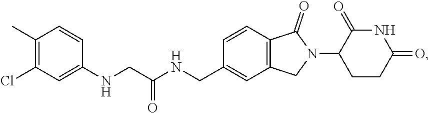 [SMILES: Cc1ccc(NCC(=O)NCc2ccc3c(c2)CN(C2CCC(=O)NC2=O)C3=O)cc1Cl]

as described in Chan, K. et al., in WO2019241274.

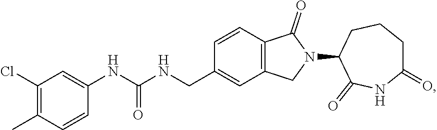 [SMILES: Cc1ccc(NC(=O)NCc2ccc3c(c2)CN([C@H]2CCCC(=O)NC2=O)C3=O)cc1Cl]

as described in Chan, K. et al., in WO2019241274.

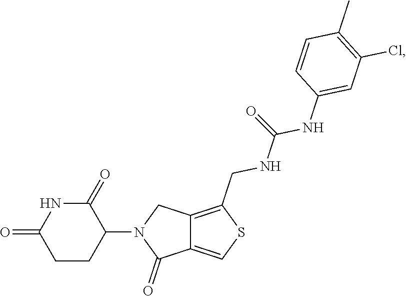 [SMILES: Cc1ccc(NC(=O)NCc2scc3c2CN(C2CCC(=O)NC2=O)C3=O)cc1Cl]

as described in Chan, K. et al., in US2018298027.

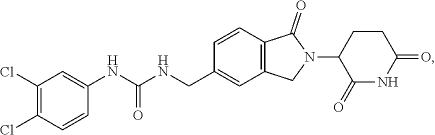 [SMILES: O=C1CCC(N2Cc3cc(CNC(=O)Nc4ccc(Cl)c(Cl)c4)ccc3C2=O)C(=O)N1]

as described in Muller, G. et al., in US2009142297.

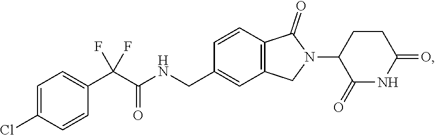 [SMILES: O=C1CCC(N2Cc3cc(CNC(=O)C(F)(F)c4ccc(Cl)cc4)ccc3C2=O)C(=O)N1]

as described in Hansen, J. et al., in WO2016007848.

In certain embodiments, the EPL is a radical of one of the above compounds, which is attached to L through a modifiable oxygen, nitrogen, or carbon atom.

In certain embodiments, the EPL is one of the following:

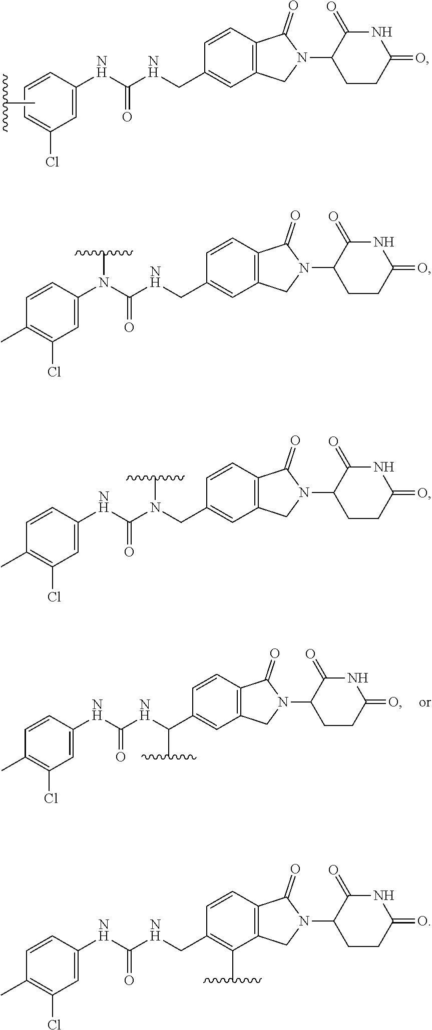

In certain embodiments, the EPL is

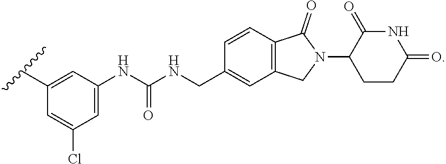 [SMILES: *c1cc(Cl)cc(NC(=O)NCc2ccc3c(c2)CN(C2CCC(=O)NC2=O)C3=O)c1]

In some embodiments, the EPL is

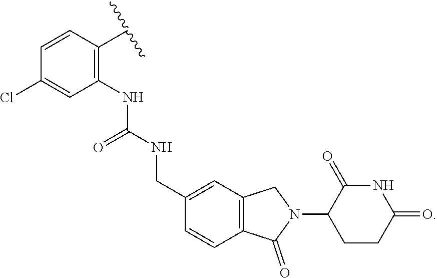 [SMILES: *c1ccc(Cl)cc1NC(=O)NCc1ccc2c(c1)CN(C1CCC(=O)NC1=O)C2=O]

In certain embodiments, the EPL is one of the following:

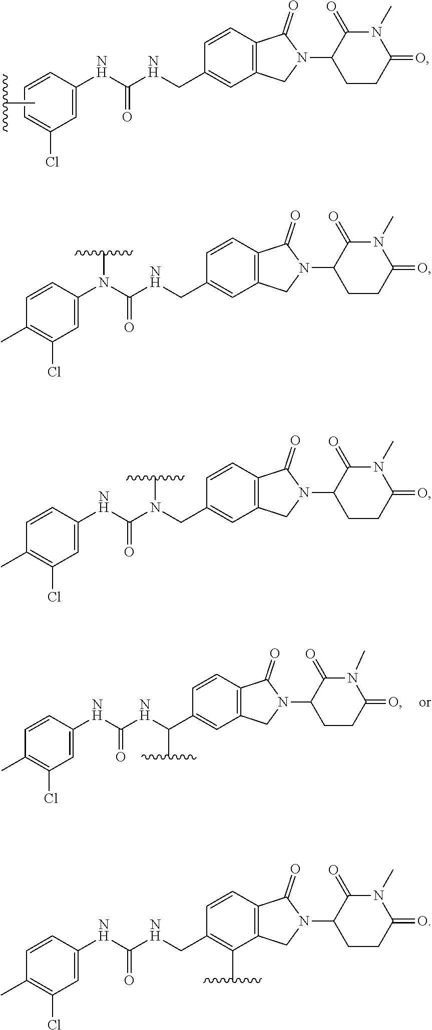

In certain embodiments, the EPL is

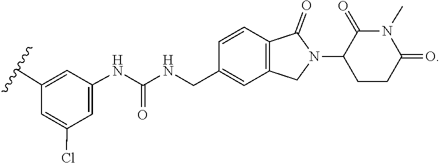 [SMILES: *c1cc(Cl)cc(NC(=O)NCc2ccc3c(c2)CN(C2CCC(=O)N(C)C2=O)C3=O)c1]

In some embodiments, the EPL is

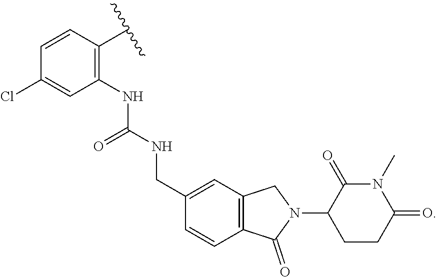 [SMILES: *c1ccc(Cl)cc1NC(=O)NCc1ccc2c(c1)CN(C1CCC(=O)N(C)C1=O)C2=O]

In some embodiments, the EPL is

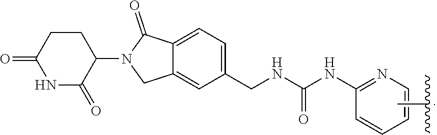

In some embodiments, the EPL is

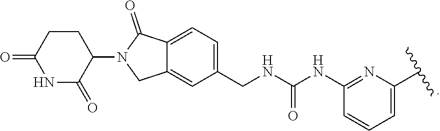 [SMILES: *c1cccc(NC(=O)NCc2ccc3c(c2)CN(C2CCC(=O)NC2=O)C3=O)n1]

In some embodiments, the EPL is

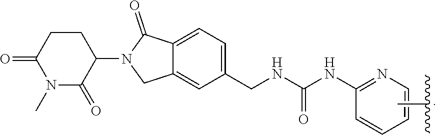

In some embodiments, the EPL is

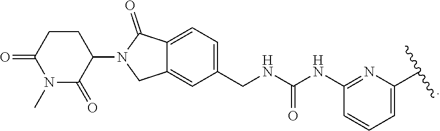 [SMILES: *c1cccc(NC(=O)NCc2ccc3c(c2)CN(C2CCC(=O)N(C)C2=O)C3=O)n1]

In certain embodiments, the EPL is

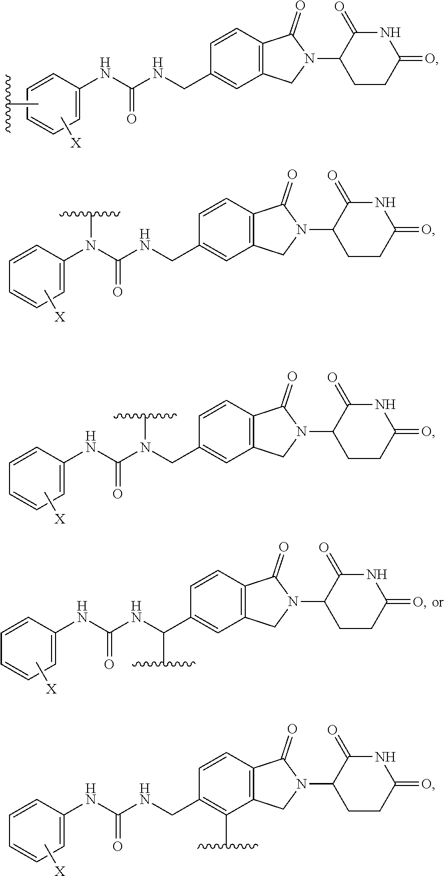

wherein X is H, D, halo, C₁₋₆ alkyl, amino, amido, amino(C₁₋₆ alkyl), C₁₋₆ alkoxy, or hydroxy.

In certain embodiments, the EPL is

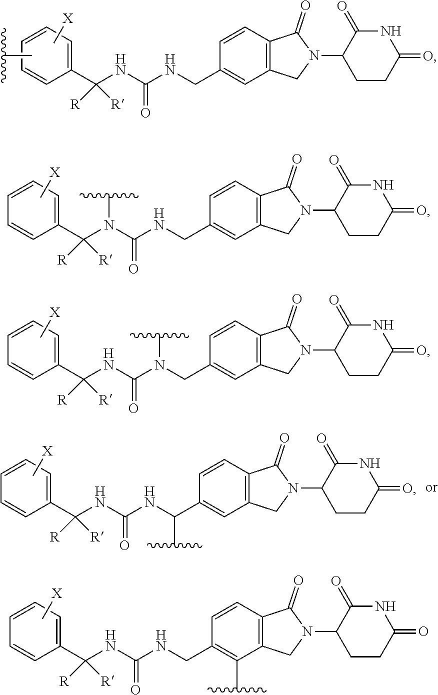

wherein X is H, D, halo, C₁₋₆ alkyl, amino, amido, amino(C₁₋₆ alkyl), C₁₋₆ alkoxy, or hydroxy; R is H, D, halo, C₁₋₆ alkyl, amino, amido, amino(C₁₋₆ alkyl), C₁₋₆ alkoxy, or hydroxy; and R′ is H, D, halo, C₁₋₆ alkyl, amino, amido, amino(C₁₋₆ alkyl), C₁₋₆ alkoxy, or hydroxy.

In certain embodiments, the EPL is

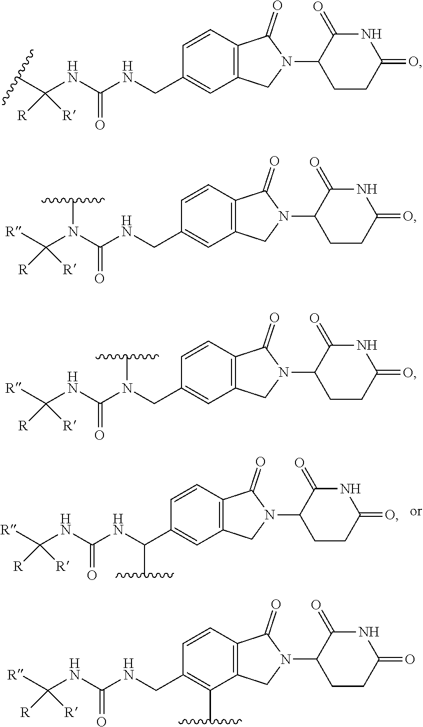

R is H, D, halo, C₁₋₆ alkyl, amino, amido, aminoalkyl, alkoxy, or hydroxy; R′ is H, D, halo, C₁₋₆ alkyl, amino, amido, amino(C₁₋₆ alkyl), C₁₋₆ alkoxy, or hydroxy; and R″ is H, D, halo, C₁₋₆ alkyl, amino, amido, amnoalkyl, C₁₋₆ alkoxy, hydroxy, aryl, 3-10 membered heteroaryl, C₃₋₇ cycloalkyl, or 3-10 membered heterocyclyl.

In certain embodiments, the EPL is

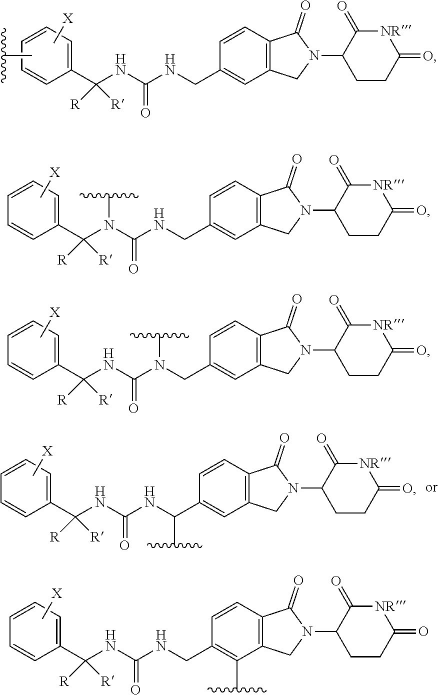

wherein X is H, D, halo, C₁₋₆ alkyl, amino, amido, amino(C₁₋₆ alkyl), C₁₋₆ alkoxy, or hydroxy; R is H, D, halo, C₁₋₆ alkyl, amino, amido, amino(C₁₋₆ alkyl), C₁₋₆ alkoxy, or hydroxy; R′ is H, D, halo, C₁₋₆ alkyl, amino, amido, amino(C₁₋₆ alkyl), C₁₋₆ alkoxy, or hydroxy; and R″′ is H or C₁₋₄ alkyl. In some embodiments, R″′ is methyl. In some embodiments, R″′ is H.

In certain embodiments, the EPL is

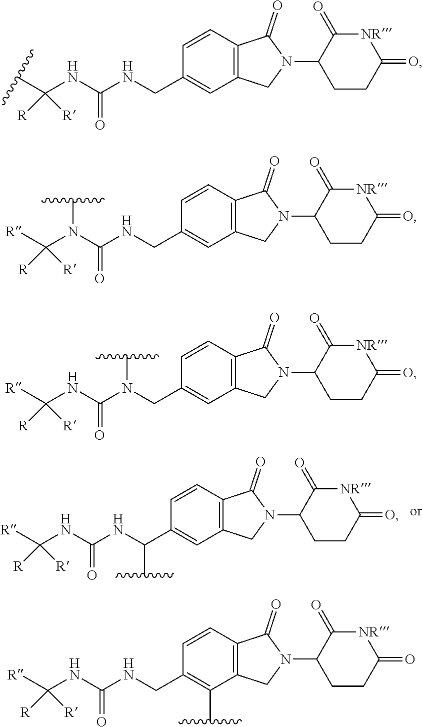

R is H, D, halo, C₁₋₆ alkyl, amino, amido, aminoalkyl, alkoxy, or hydroxy; R′ is H, D, halo, C₁₋₆ alkyl, amino, amido, amino(C₁₋₆ alkyl), C₁₋₆ alkoxy, or hydroxy; R″ is H, D, halo, C₁₋₆ alkyl, amino, amido, aminoalkyl, C₁₋₆ alkoxy, hydroxy, aryl, 3-10 membered heteroaryl, C₃₋₇ cycloalkyl, or 3-10 membered heterocyclyl; and R″′ is H or C₁₋₄ alkyl. In some embodiments, R″′ is methyl. In some embodiments, R″′ is H.

In certain embodiments, the EPL is one of the following:

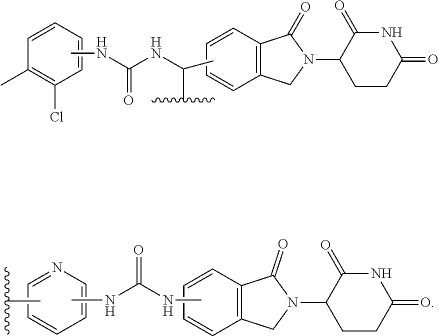

In certain embodiments, the EPL is one of the following:

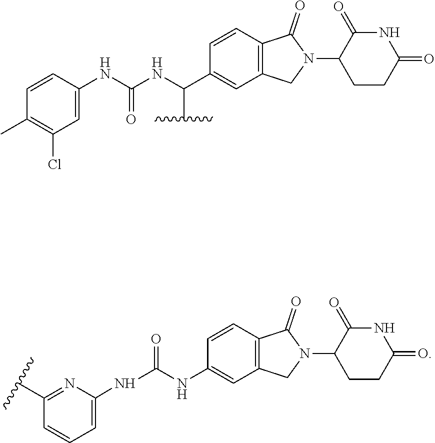 [SMILES: *C(NC(=O)Nc1ccc(C)c(Cl)c1)c1ccc2c(c1)CN(C1CCC(=O)NC1=O)C2=O ; *c1cccc(NC(=O)Nc2ccc3c(c2)CN(C2CCC(=O)NC2=O)C3=O)n1]

### B. Moiety for Cyclin K

In certain embodiments, the EPL is a moiety that binds to or degrades Cyclin K. Exemplary compounds that bind to and/or degrade Cyclin K are reported in the literature, including:

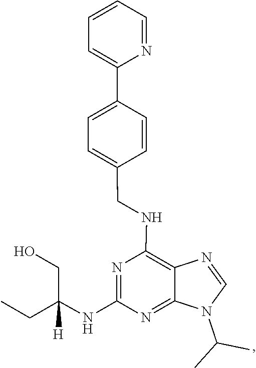 [SMILES: CC[C@H](CO)Nc1nc(NCc2ccc(-c3ccccn3)cc2)c2ncn(C(C)C)c2n1]

as described in Slabicki, M. et al., in Nature (London, United Kingdom) (2020), 585(7824): 293.

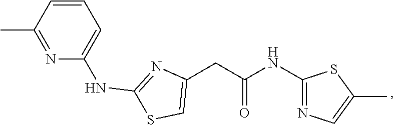

as described in Lv, L. et al., in eLife (2020), 9: e59994.

In certain embodiments, the EPL is a radical of one of the above compounds, which is attached to L through a modifiable oxygen, nitrogen, or carbon atom.

In certain embodiments, the EPL is one of the following:

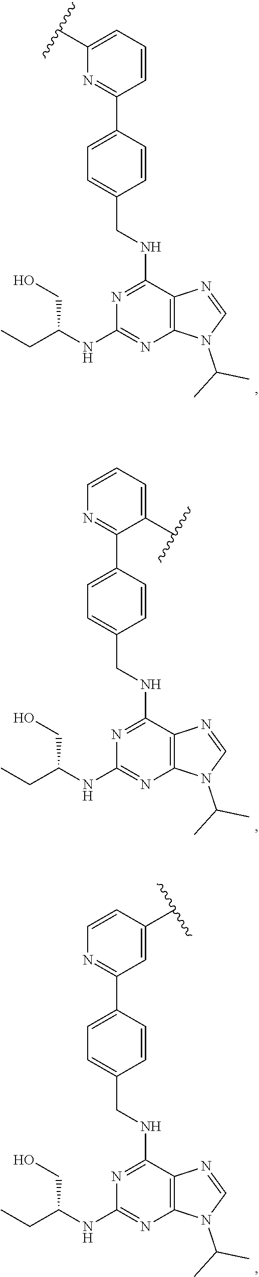

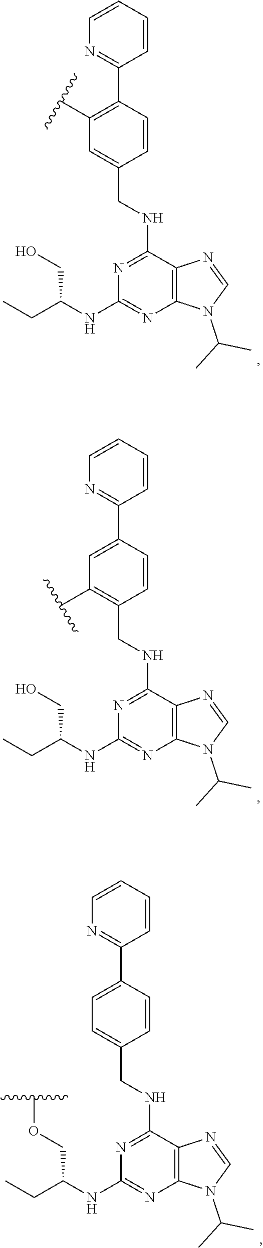

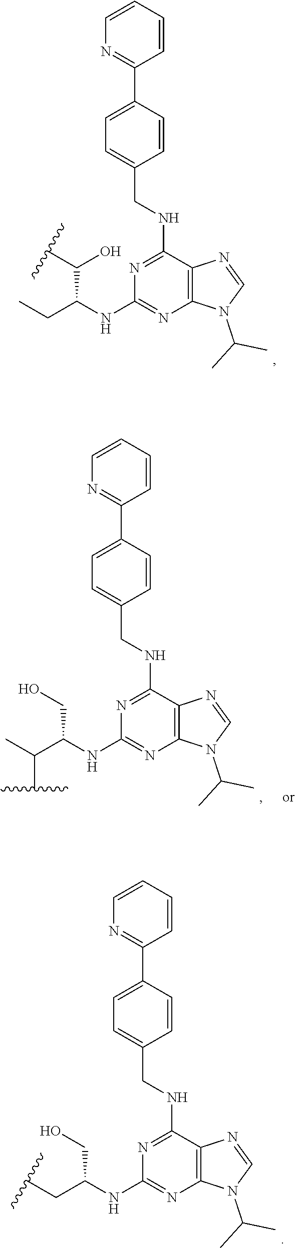

### C. Moiety for RBM39

In certain embodiments, the EPL is a moiety that binds to or degrades RBM39. Exemplary compounds that bind to and/or degrade RBM39 are reported in the literature, including:

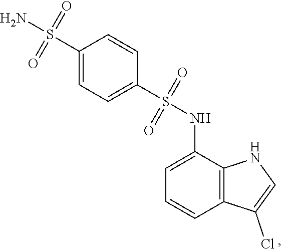 [SMILES: NS(=O)(=O)c1ccc(S(=O)(=O)Nc2cccc3c(Cl)c[nH]c23)cc1]

as described in Han, T. et al., Science (2017), 356(6336): 3755.

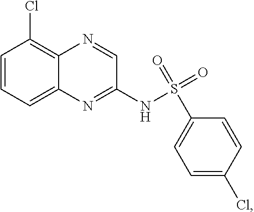 [SMILES: O=S(=O)(Nc1cnc2c(Cl)cccc2n1)c1ccc(Cl)cc1]

as described in Han, T. et al., Science (2017), 356(6336): 3755.

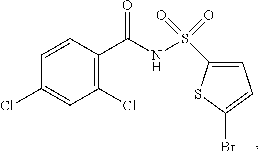 [SMILES: O=C(NS(=O)(=O)c1ccc(Br)s1)c1ccc(Cl)cc1Cl]

as described in Han, T. et al., Science (2017), 356(6336): 3755.

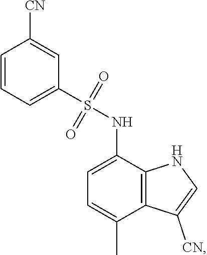 [SMILES: Cc1ccc(NS(=O)(=O)c2cccc(C#N)c2)c2[nH]cc(C#N)c12]

as described in Uehara, T. et al., Nat. Chem. Bio (2017), 13: 675.

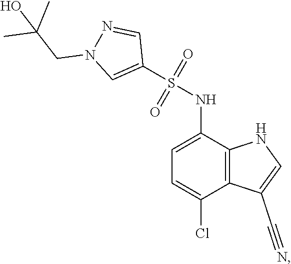 [SMILES: CC(C)(O)Cn1cc(S(=O)(=O)Nc2ccc(Cl)c3c(C#N)c[nH]c23)cn1]

as described in Estrada, M. et al., WO2020210139.

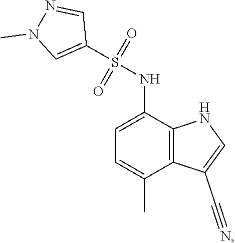 [SMILES: Cc1ccc(NS(=O)(=O)c2cnn(C)c2)c2[nH]cc(C#N)c12]

as described in Estrada, M. et al., WO2020210139.

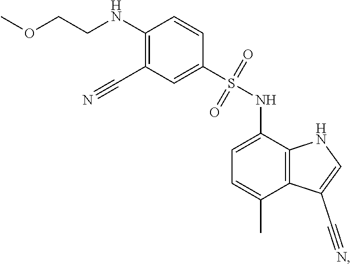 [SMILES: COCCNc1ccc(S(=O)(=O)Nc2ccc(C)c3c(C#N)c[nH]c23)cc1C#N]

as described in Gray, N. et al., WO2019147783.

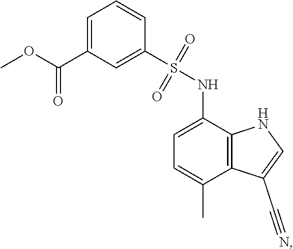 [SMILES: COC(=O)c1cccc(S(=O)(=O)Nc2ccc(C)c3c(C#N)c[nH]c23)c1]

as described in Gray, N. et al., WO2019147783.

In certain embodiments, the EPL is a radical of one of the above compounds, which is attached to L through a modifiable oxygen, nitrogen, or carbon atom.

### D. Moiety for RBM23

In certain embodiments, the EPL is a moiety that binds to and/or degrades RBM23. Exemplary compounds that bind to and/or degrade RBM23 are reported in the literature, including:

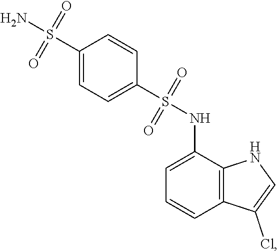 [SMILES: NS(=O)(=O)c1ccc(S(=O)(=O)Nc2cccc3c(Cl)c[nH]c23)cc1]

as described in Ting, T. et al., in Cell Reports (2019) 29: 1499.

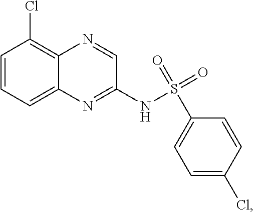 [SMILES: O=S(=O)(Nc1cnc2c(Cl)cccc2n1)c1ccc(Cl)cc1]

as described in Ting, T. et al., Cell Reports (2019) 29: 1499.

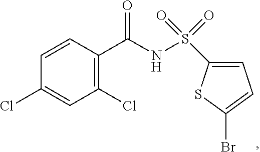 [SMILES: O=C(NS(=O)(=O)c1ccc(Br)s1)c1ccc(Cl)cc1Cl]

as that described in Ting, T. et al., Cell Reports (2019) 29: 1499.

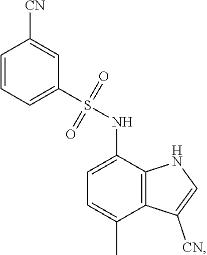 [SMILES: Cc1ccc(NS(=O)(=O)c2cccc(C#N)c2)c2[nH]cc(C#N)c12]

as described in Ting, T. et al., Cell Reports (2019) 29: 1499.

In certain embodiments, the EPL is a radical of one of the above compounds, which is attached to L through a modifiable oxygen, nitrogen, or carbon atom.

### E. Moiety for IKZF1

In certain embodiments, the EPL is a moiety that binds to and/or degrades DNA-Binding Protein Ikaros (IKZF1). Exemplary compounds that bind to and/or degrade IKZF1 are reported in the literature, including:

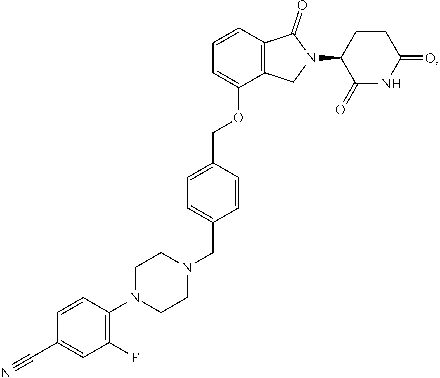 [SMILES: N#Cc1ccc(N2CCN(Cc3ccc(COc4cccc5c4CN([C@H]4CCC(=O)NC4=O)C5=O)cc3)CC2)c(F)c1]

as described in Alexander, M. D. et al., WO2019/014100.

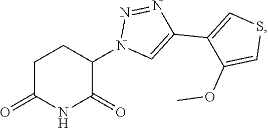 [SMILES: COc1cscc1-c1cn(C2CCC(=O)NC2=O)nn1]

as described in Watanabe, M. et al., WO2019/146773.

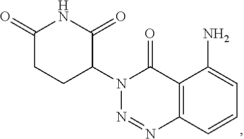 [SMILES: Nc1cccc2nnn(C3CCC(=O)NC3=O)c(=O)c12]

as described in Hwang, J. et al., WO2018/208123.

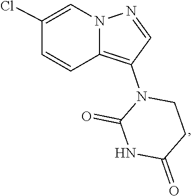 [SMILES: O=C1CCN(c2cnn3cc(Cl)ccc23)C(=O)N1]

as described in Axford, J. et al., WO2021/053555.

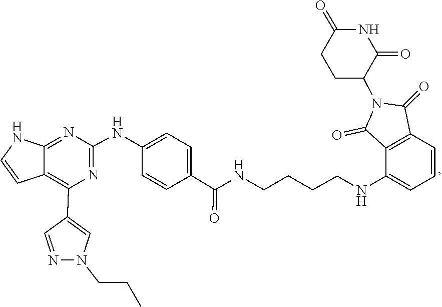 [SMILES: CCCn1cc(-c2nc(Nc3ccc(C(=O)NCCCCNc4cccc5c4C(=O)N(C4CCC(=O)NC4=O)C5=O)cc3)nc3[nH]ccc23)cn1]

as described in Min, J. et al., WO2021/022076.

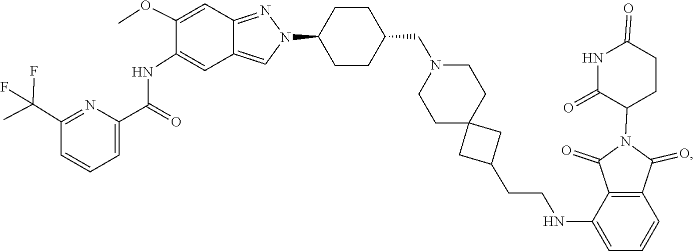 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cccc(C(C)(F)F)n1]

as described in Mainolfi, N. et al., WO2020/264499.

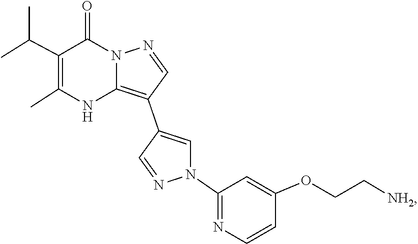 [SMILES: Cc1[nH]c2c(-c3cnn(-c4cc(OCCN)ccn4)c3)cnn2c(=O)c1C(C)C]

as described in Qi, J. et al., WO2020/263832.

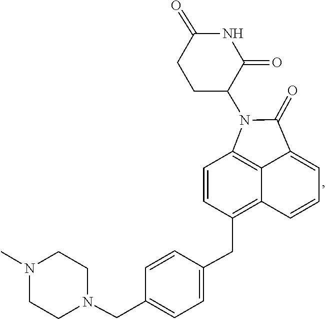 [SMILES: CN1CCN(Cc2ccc(Cc3ccc4c5c(cccc35)C(=O)N4C3CCC(=O)NC3=O)cc2)CC1]

as described in Henderson, J. et al., WO2020/210630.

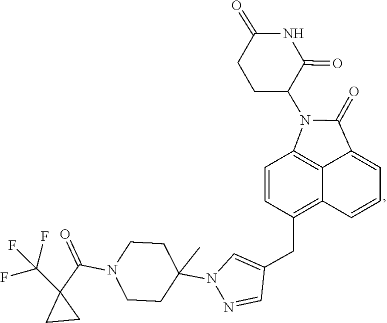 [SMILES: CC1(n2cc(Cc3ccc4c5c(cccc35)C(=O)N4C3CCC(=O)NC3=O)cn2)CCN(C(=O)C2(C(F)(F)F)CC2)CC1]

as described in Henderson, J. et al., WO2020/210630.

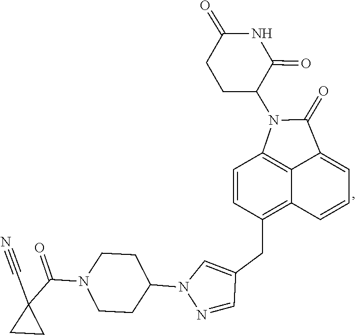 [SMILES: N#CC1(C(=O)N2CCC(n3cc(Cc4ccc5c6c(cccc46)C(=O)N5C4CCC(=O)NC4=O)cn3)CC2)CC1]

as described in Henderson, J. et al., WO2020/210630.

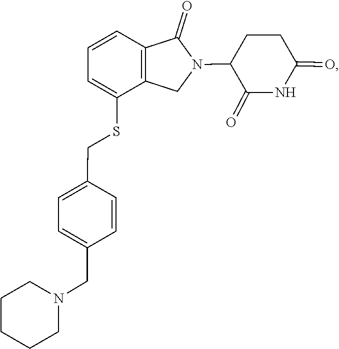 [SMILES: O=C1CCC(N2Cc3c(SCc4ccc(CN5CCCCC5)cc4)cccc3C2=O)C(=O)N1]

as described in Yang, X. et al., WO2020/173426.

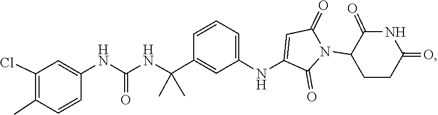 [SMILES: Cc1ccc(NC(=O)NC(C)(C)c2cccc(NC3=CC(=O)N(C4CCC(=O)NC4=O)C3=O)c2)cc1Cl]

as described in Verano, A. et al., WO2020/117759.

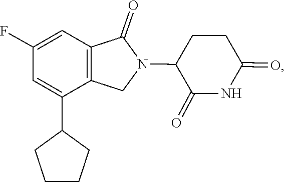 [SMILES: O=C1CCC(N2Cc3c(cc(F)cc3C3CCCC3)C2=O)C(=O)N1]

as described in Chan, K. et al., WO2020/102195.

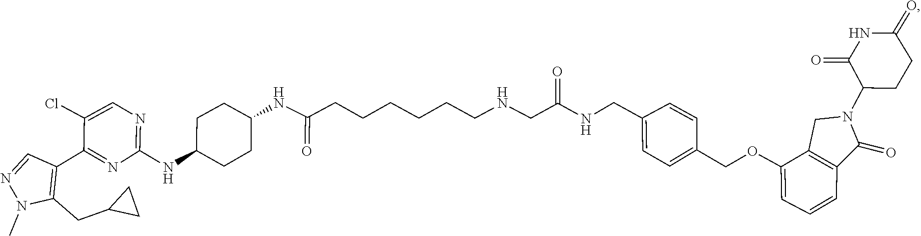 [SMILES: Cn1ncc(-c2nc(N[C@H]3CC[C@H](NC(=O)CCCCCCNCC(=O)NCc4ccc(COc5cccc6c5CN(C5CCC(=O)NC5=O)C6=O)cc4)CC3)ncc2Cl)c1CC1CC1]

as described in Chan, K. et al., WO2020/023782.

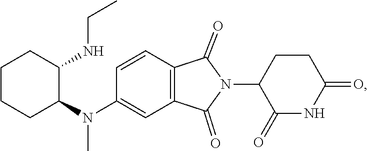 [SMILES: CCN[C@H]1CCCC[C@@H]1N(C)c1ccc2c(c1)C(=O)N(C1CCC(=O)NC1=O)C2=O]

as described in Beckwith, R et al., WO2020/012337.

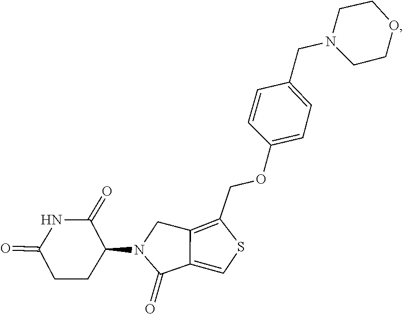 [SMILES: O=C1CC[C@H](N2Cc3c(csc3COc3ccc(CN4CCOCC4)cc3)C2=O)C(=O)N1]

as described in Chan, K. et al., WO2019/241271.

In certain embodiments, the EPL is a radical of one of the above compounds, which is attached to L through a modifiable oxygen, nitrogen, or carbon atom.

### F. Moiety for IKZF3

In certain embodiments, the EPL is a moiety that binds to and/or degrades Zinc Finger Protein Aiolos (IKZF3). Exemplary compounds that bind to and/or degrade IKZF3 are reported in the literature, including:

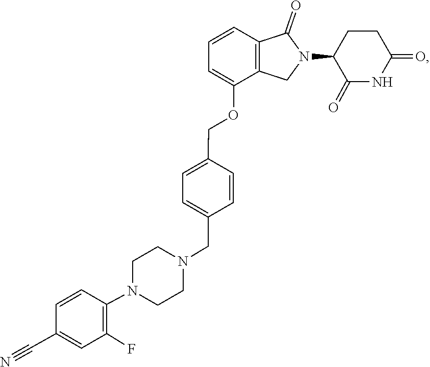 [SMILES: N#Cc1ccc(N2CCN(Cc3ccc(COc4cccc5c4CN([C@H]4CCC(=O)NC4=O)C5=O)cc3)CC2)c(F)c1]

as described in Alexander, M. D. et al., WO2019/014100.

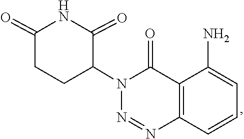 [SMILES: Nc1cccc2nnn(C3CCC(=O)NC3=O)c(=O)c12]

as described in Hwang, J. et al., WO2018/208123.

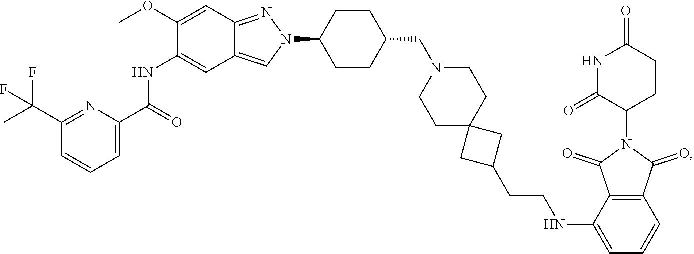 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cccc(C(C)(F)F)n1]

as described in Mainolfi, N. et al., WO2020/264499.

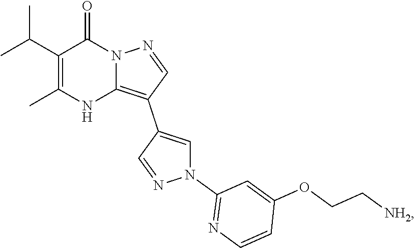 [SMILES: Cc1[nH]c2c(-c3cnn(-c4cc(OCCN)ccn4)c3)cnn2c(=O)c1C(C)C]

as described in Qi, J. et al., WO2020/263832.

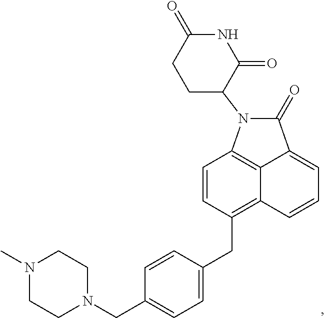 [SMILES: CN1CCN(Cc2ccc(Cc3ccc4c5c(cccc35)C(=O)N4C3CCC(=O)NC3=O)cc2)CC1]

as described in Henderson, J. et al., WO2020/210630.

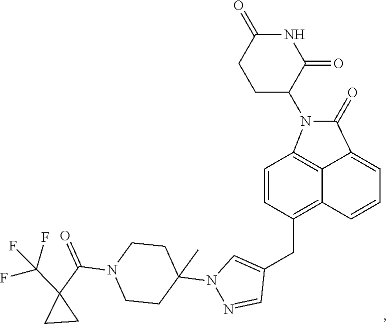 [SMILES: CC1(n2cc(Cc3ccc4c5c(cccc35)C(=O)N4C3CCC(=O)NC3=O)cn2)CCN(C(=O)C2(C(F)(F)F)CC2)CC1]

as described in Henderson, J. et al., WO2020/210630.

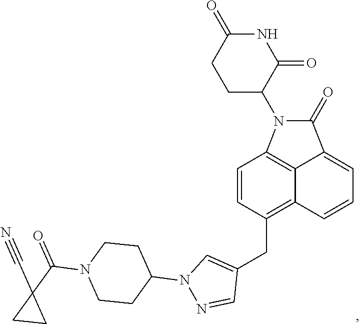 [SMILES: N#CC1(C(=O)N2CCC(n3cc(Cc4ccc5c6c(cccc46)C(=O)N5C4CCC(=O)NC4=O)cn3)CC2)CC1]

as described in Henderson, J. et al., WO2020/210630.

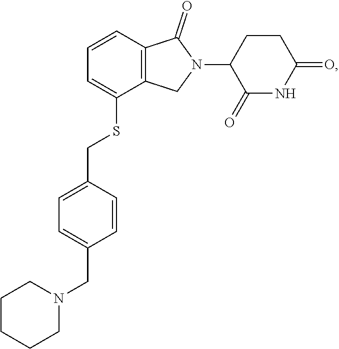 [SMILES: O=C1CCC(N2Cc3c(SCc4ccc(CN5CCCCC5)cc4)cccc3C2=O)C(=O)N1]

as described in Yang, X. et al., WO2020/173426.

In certain embodiments, the EPL is a radical of one of the above compounds, which is attached to L through a modifiable oxygen, nitrogen, or carbon atom.

### G. Moiety for CDK4

In certain embodiments, the EPL is a moiety that binds to and/or degrades Cyclin-dependent kinase 4 (CDK4). Exemplary compounds that bind to and/or degrade CDK4 are reported in the literature, including:

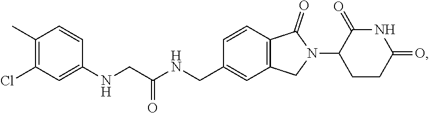 [SMILES: Cc1ccc(NCC(=O)NCc2ccc3c(c2)CN(C2CCC(=O)NC2=O)C3=O)cc1Cl]

as described in Zhao, M. et al., Biochem Biophys Res Comm (2021), 549 (21): 150.

In certain embodiments, the EPL is a radical of the above compound, which is attached to L through a modifiable oxygen, nitrogen, or carbon atom.

### H. Moiety for PLK1

In certain embodiments, the EPL is a moiety that binds to and/or degrades Polo-like kinase 1 (PLK1). Exemplary compounds that bind to and/or degrade PLK1 are reported in the literature, including:

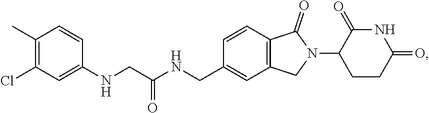 [SMILES: Cc1ccc(NCC(=O)NCc2ccc3c(c2)CN(C2CCC(=O)NC2=O)C3=O)cc1Cl]

as described in Li, L. et al., Mol Ther Onc (2020), 18: 215.

In certain embodiments, the EPL is a radical of the above compound, which is attached to L through a modifiable oxygen, nitrogen, or carbon atom.

### Part C: Exemplary Further Description of TPL Component of Compounds of Formula I and II

Compounds of Formula I and II may be further characterized according to, for example, the identity of the TPL component. As generally described above, the TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα.

In certain embodiments, TPL is a moiety that binds to a target protein selected from KRAS, HER2, BTK, EGFR, androgen receptor protein, estrogen receptor protein, or ALK. In certain embodiments, TPL is a moiety that binds to a target protein selected from IDH1, FLT3, FGFR1, FGFR4, HCV-NS3, FGFR2, FGFR3, ERK1, ERK2, FGR, HER3, HER4, or PI3Kα. In certain embodiments, TPL is a moiety that binds KRAS. In certain embodiments, TPL is a moiety that binds HER2. In certain embodiments, TPL is a moiety that binds BTK. In certain embodiments, TPL is a moiety that binds EGFR. In certain embodiments, TPL is a moiety that binds androgen receptor protein. In certain embodiments, TPL is a moiety that binds estrogen receptor protein. In certain embodiments, TPL is a moiety that binds ALK. In certain embodiments, TPL is a moiety that binds IDH1. In certain embodiments, TPL is a moiety that binds FLT3. In certain embodiments, TPL is a moiety that binds FGFR1. In certain embodiments, TPL is a moiety that binds FGFR4. In certain embodiments, TPL is a moiety that binds HCV-NS3. In certain embodiments, TPL is a moiety that binds FGFR2. In certain embodiments, TPL is a moiety that binds FGFR3. In certain embodiments, TPL is a moiety that binds ERK1. In certain embodiments, TPL is a moiety that binds ERK2. In certain embodiments, TPL is a moiety that binds FGR. In certain embodiments, TPL is a moiety that binds HER3. In certain embodiments, TPL is a moiety that binds HER4. In certain embodiments, TPL is a moiety that binds PI3Kα.

Exemplary moieties for the TPL component are described in more detail below.

### Moiety for HER2

In certain embodiments, the TPL is a moiety that binds to HER2. Exemplary compounds that bind to HER2 are reported in the literature. A radical of such compounds reported in the literature that bind HER2 are amenable for use in the present invention.

In certain embodiments, the TPL is one of the following

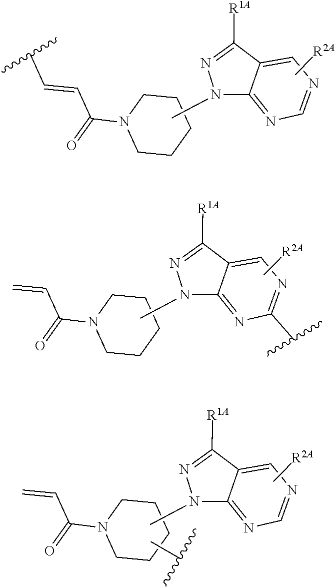

wherein:
R^{1A} is —C(O)(NR^{5A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R⁵)(R⁶))
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to the same nitrogen atom are taken together with the nitrogen atom to which they are attached to form a 3-7 membered heterocyclic ring.

In certain embodiments, the TPL is one of the following:

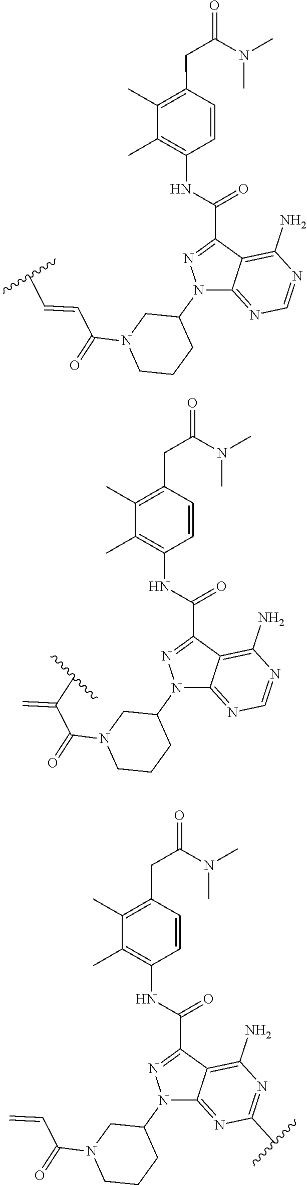

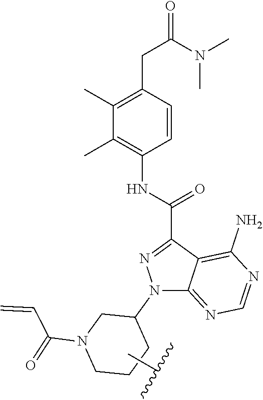

In certain embodiments, the TPL is a radical of

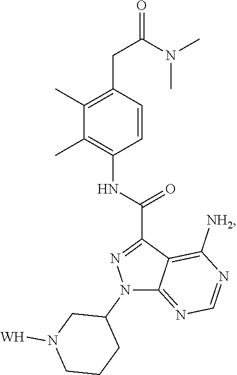

where WH is a group that reacts with HER2 to form a covalent linkage.

In certain embodiments, the TPL is one of the following:

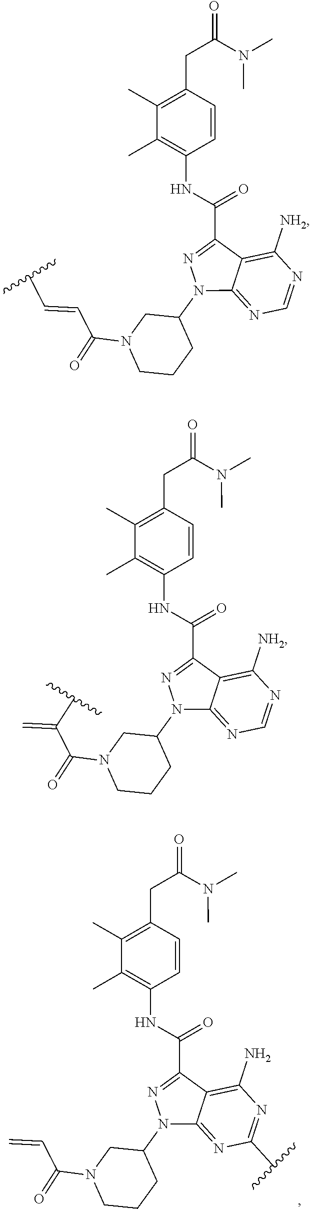 [SMILES: */C=C/C(=O)N1CCCC(n2nc(C(=O)Nc3ccc(CC(=O)N(C)C)c(C)c3C)c3c(N)ncnc32)C1 ; *C(=C)C(=O)N1CCCC(n2nc(C(=O)Nc3ccc(CC(=O)N(C)C)c(C)c3C)c3c(N)ncnc32)C1 ; *c1nc(N)c2c(C(=O)Nc3ccc(CC(=O)N(C)C)c(C)c3C)nn(C3CCCN(C(=O)C=C)C3)c2n1]

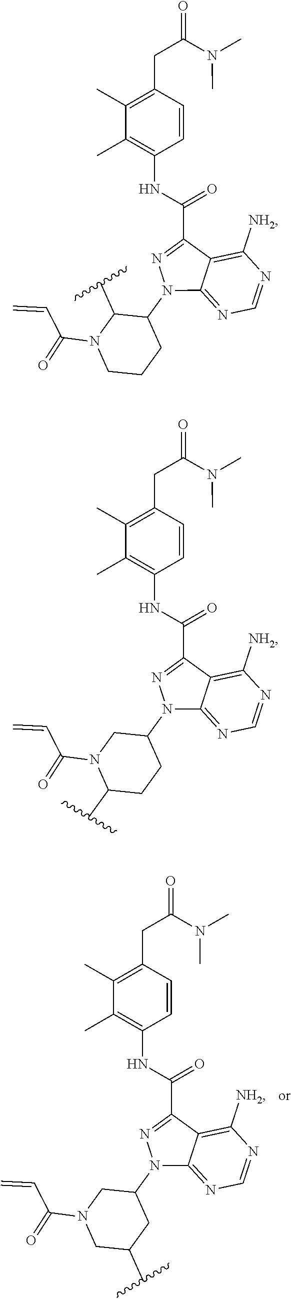 [SMILES: *C1C(n2nc(C(=O)Nc3ccc(CC(=O)N(C)C)c(C)c3C)c3c(N)ncnc32)CCCN1C(=O)C=C ; *C1CCC(n2nc(C(=O)Nc3ccc(CC(=O)N(C)C)c(C)c3C)c3c(N)ncnc32)CN1C(=O)C=C ; *C1CC(n2nc(C(=O)Nc3ccc(CC(=O)N(C)C)c(C)c3C)c3c(N)ncnc32)CN(C(=O)C=C)C1 ; "or"]

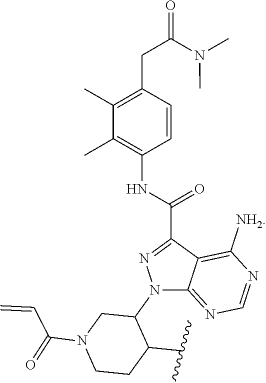 [SMILES: *C1CCN(C(=O)C=C)CC1n1nc(C(=O)Nc2ccc(CC(=O)N(C)C)c(C)c2C)c2c(N)ncnc21]

In certain embodiments, the TPL is a moiety that inhibits and/or binds human epidermal growth factor receptor 2 (HER2). Compounds that inhibit and/or bind to HER2 are reported in the literature, which include:

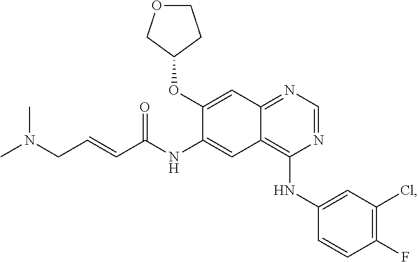 [SMILES: CN(C)C/C=C/C(=O)Nc1cc2c(Nc3ccc(F)c(Cl)c3)ncnc2cc1O[C@H]1CCOC1]

as described in Chen, J. et al., WO 2015/023703;

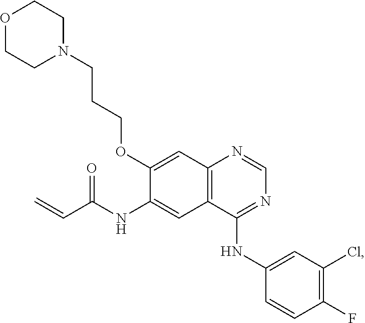 [SMILES: C=CC(=O)Nc1cc2c(Nc3ccc(F)c(Cl)c3)ncnc2cc1OCCCN1CCOCC1]

as described in Huang, Z. et al., WO 2012/027960;

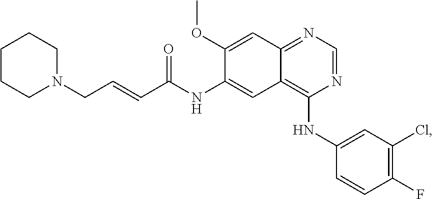 [SMILES: COc1cc2ncnc(Nc3ccc(F)c(Cl)c3)c2cc1NC(=O)/C=C/CN1CCCCC1]

as described in Wu, F. et al., WO 2012/159457;

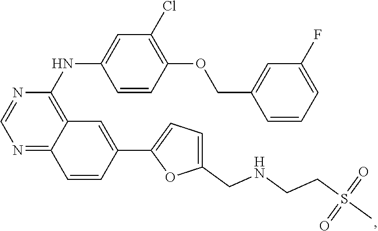 [SMILES: CS(=O)(=O)CCNCc1ccc(-c2ccc3ncnc(Nc4ccc(OCc5cccc(F)c5)c(Cl)c4)c3c2)o1]

as described in Wu, F. et al., WO 2012/159457;

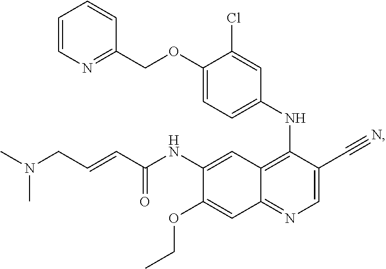 [SMILES: CCOc1cc2ncc(C#N)c(Nc3ccc(OCc4ccccn4)c(Cl)c3)c2cc1NC(=O)/C=C/CN(C)C]

as described in Wissner, A. et al., WO 2005/034955;

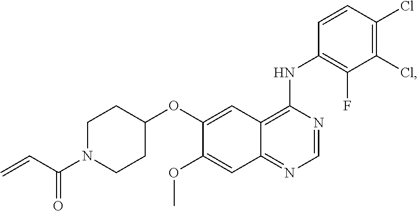 [SMILES: C=CC(=O)N1CCC(Oc2cc3c(Nc4ccc(Cl)c(Cl)c4F)ncnc3cc2OC)CC1]

as described in Li, Z. et al., WO 2019/149164;

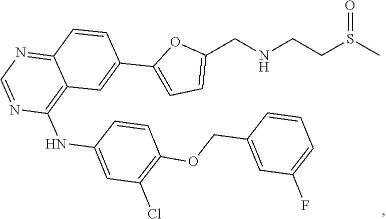 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

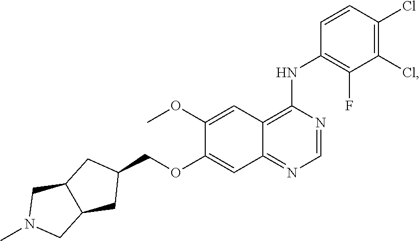 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

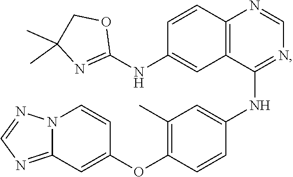 [SMILES: Cc1cc(Nc2ncnc3ccc(NC4=NC(C)(C)CO4)cc23)ccc1Oc1ccn2ncnc2c1]

as described in Xia, G. et al., WO 2017/148391;

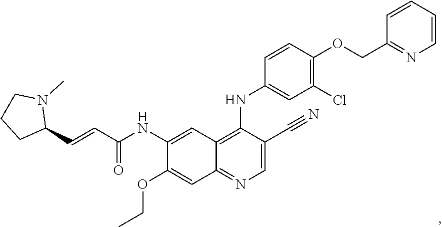 [SMILES: CCOc1cc2ncc(C#N)c(Nc3ccc(OCc4ccccn4)c(Cl)c3)c2cc1NC(=O)/C=C/[C@H]1CCCN1C]

as described in Li, X. et al., WO 2012/122865.

In certain embodiments, the TPL is a radical of one of the above compounds, which is attached to L through a modifiable oxygen, nitrogen, or carbon atom.

### Moiety for BTK

In certain embodiments, the TPL is a moiety that binds to Bruton's tyrosine kinase (BTK). Exemplary compounds that bind to BTK are reported in the literature, such as ibrutinib and zanubrutinib. A radical of such compounds reported in the literature that bind BTK are amenable for use in the present invention.

In certain embodiments, the TPL is one of the following:

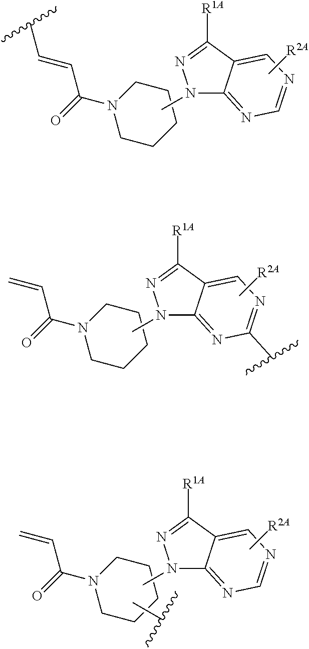

wherein:
R^{1A} is -(phenyl optionally substituted with 1, 2, 3, or 5 substituents independently selected from halo, hydroxyl, C₁₋₄ alkyl, and C₁₋₄ alkoxyl)-O-(phenyl optionally substituted with 1, 2, 3, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is:

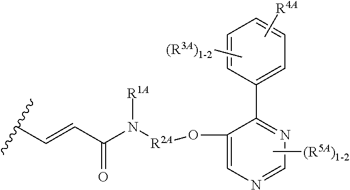

wherein:
R^{1A}, R^{6A}, and R^{7A} are independently hydrogen or C₁₋₄ alkyl;
R^{2A} is C₁₋₄ alkylene;
R^{3A} and R^{5A} each represent independently for each occurrence hydrogen, halo, hydroxyl, C₁₋₄ alkyl, C₁₋₄ alkoxyl, or —N(R^{8A})(R^{9A});
R^{4A} is —N(R^{6A})C(O)R^{7A};
R^{7A} is phenyl or 5-6 membered heteroaryl containing 1, 2, or 3 heteroatoms selected from oxygen, nitrogen, or sulfur, wherein the phentyl and heteroaryl are substituted with 0, 1, 2, or 3 substituents independently selected from the group consisting of halo, hydroxyl, C₁₋₄ alkyl, C₃₋₇ cycloalkyl, C₁₋₄ alkoxyl, and —N(R^{8A})(R^{9A});
R^{8A} and R^{9A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{8A} and R^{9A} attached to same nitrogen atom are taken together with the nitrogen atom to which they are attached to form a 3-7 membered heterocyclic ring.

In certain embodiments, the TPL is one of the following:

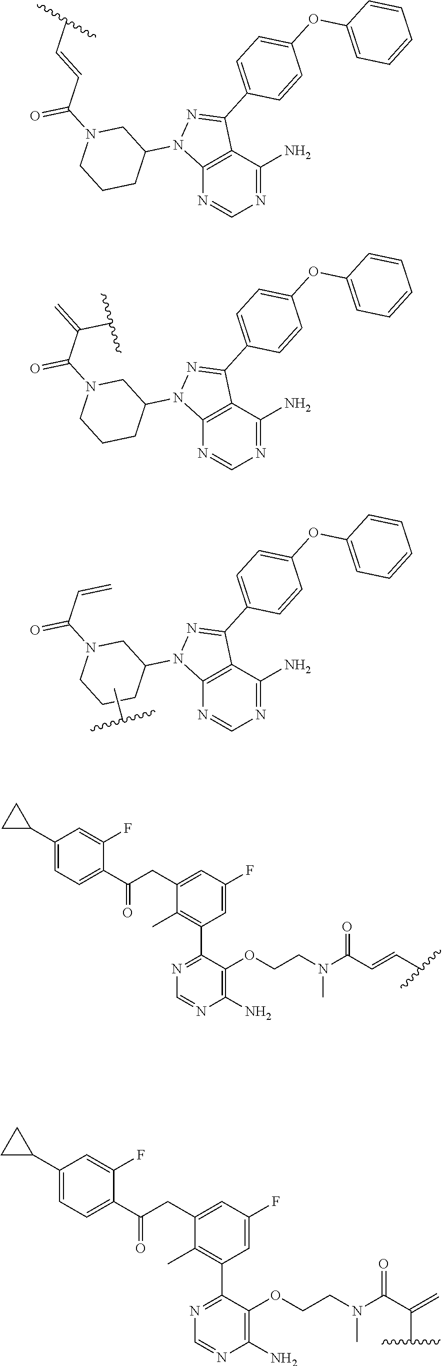

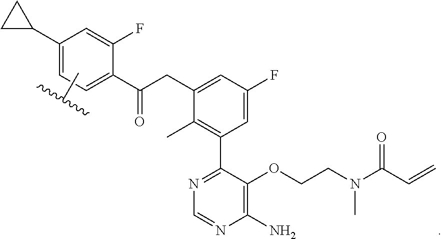

In certain embodiments, the TPL is a radical of

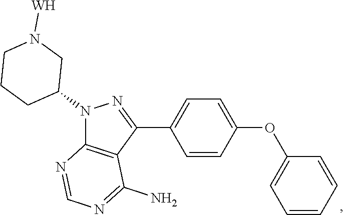

wherein WH is a group that reacts with BTK to form a covalent linkage. In certain embodiments, the TPL is a radical of

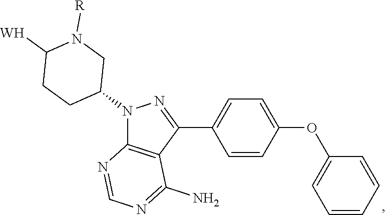

wherein WH is a group that reacts with BTK to form a covalent linkage, and R is H, alkyl, or acyl.

In certain embodiments, the TPL is one of the following:

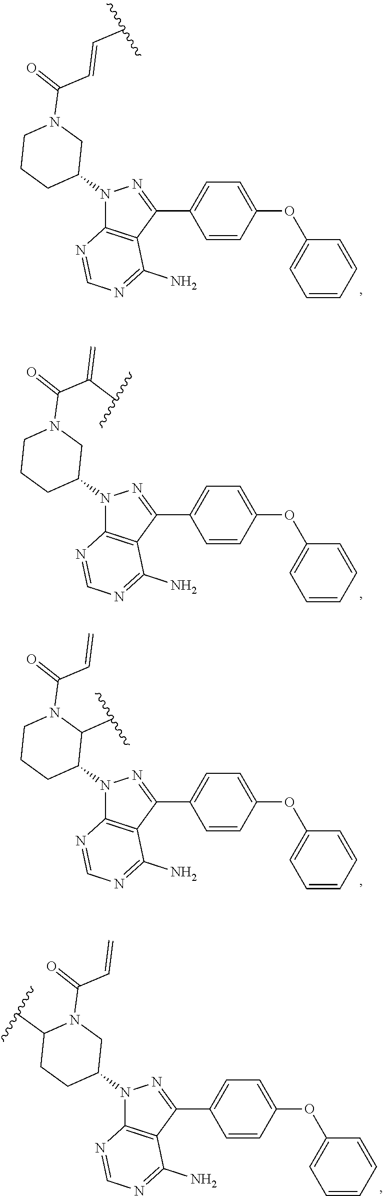

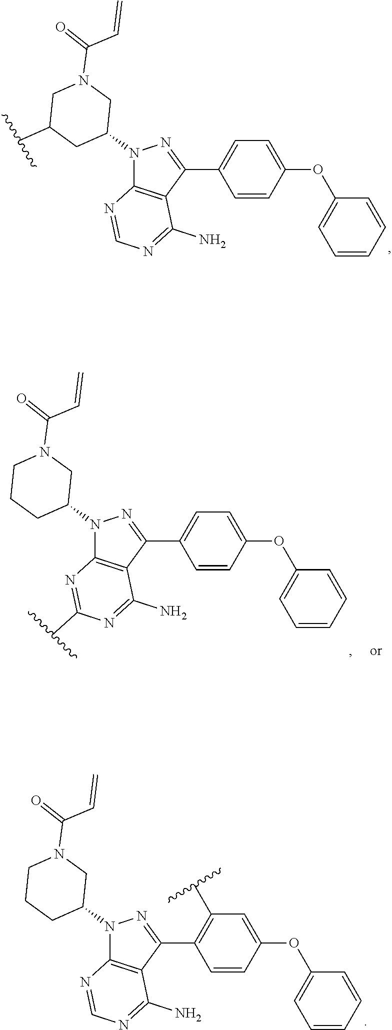 [SMILES: *C1C[C@@H](n2nc(-c3ccc(Oc4ccccc4)cc3)c3c(N)ncnc32)CN(C(=O)C=C)C1 ; *c1nc(N)c2c(-c3ccc(Oc4ccccc4)cc3)nn([C@@H]3CCCN(C(=O)C=C)C3)c2n1 ; *c1cc(Oc2ccccc2)ccc1-c1nn([C@@H]2CCCN(C(=O)C=C)C2)c2ncnc(N)c12]

In certain embodiments, the TPL is a radical of

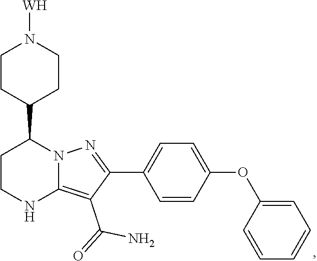

wherein WH is a group that reacts with BTK to form a covalent linkage.

In certain embodiments, the TPL is a radical of

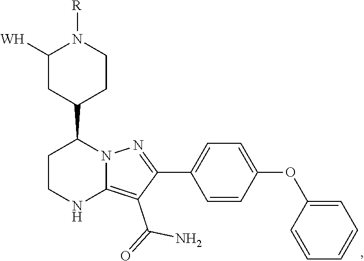

wherein WH is a group that reacts with BTK to form a covalent linkage, and R is H, alkyl, or acyl.

In certain embodiments, the TPL is one of the following:

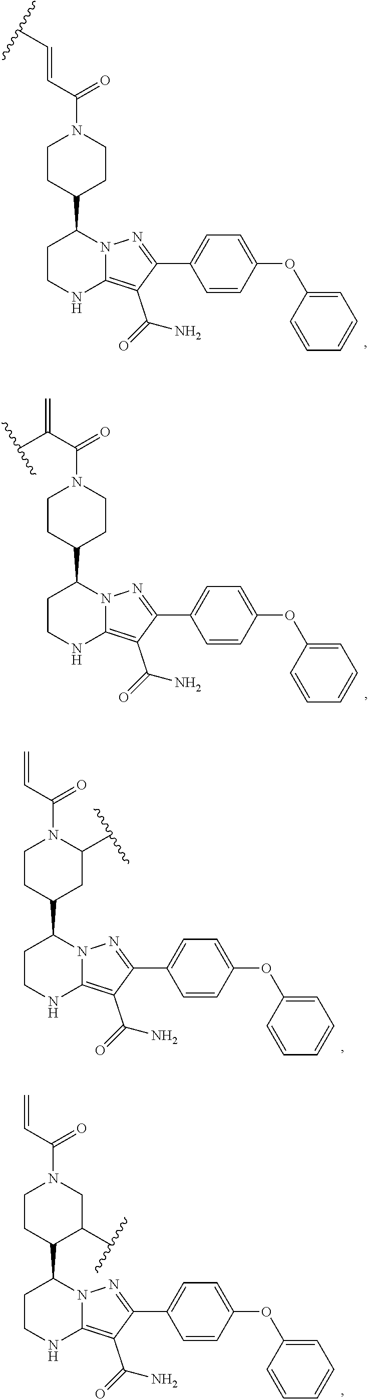 [SMILES: */C=C/C(=O)N1CCC([C@@H]2CCNc3c(C(N)=O)c(-c4ccc(Oc5ccccc5)cc4)nn32)CC1 ; *C(=C)C(=O)N1CCC([C@@H]2CCNc3c(C(N)=O)c(-c4ccc(Oc5ccccc5)cc4)nn32)CC1 ; *C1CC([C@@H]2CCNc3c(C(N)=O)c(-c4ccc(Oc5ccccc5)cc4)nn32)CCN1C(=O)C=C ; *C1CN(C(=O)C=C)CCC1[C@@H]1CCNc2c(C(N)=O)c(-c3ccc(Oc4ccccc4)cc3)nn21]

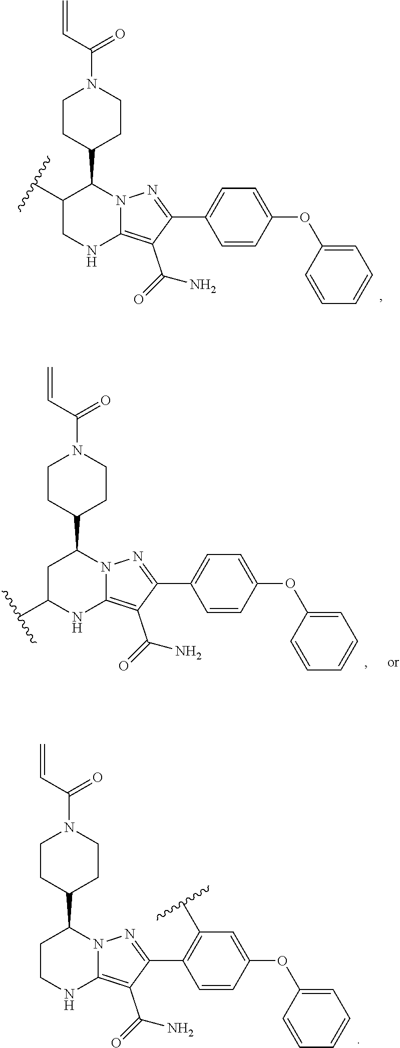 [SMILES: *C1CNc2c(C(N)=O)c(-c3ccc(Oc4ccccc4)cc3)nn2[C@@H]1C1CCN(C(=O)C=C)CC1 ; *C1C[C@@H](C2CCN(C(=O)C=C)CC2)n2nc(-c3ccc(Oc4ccccc4)cc3)c(C(N)=O)c2N1 ; *c1cc(Oc2ccccc2)ccc1-c1nn2c(c1C(N)=O)NCC[C@H]2C1CCN(C(=O)C=C)CC1]

In certain embodiments, the TPL is a radical of

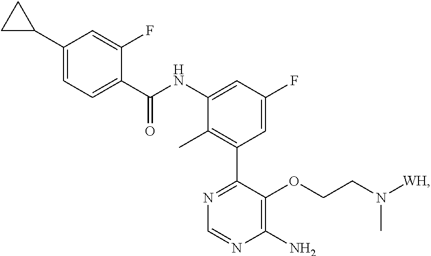

wherein WH is a group that reacts with BTK to form a covalent linkage.

In certain embodiments, the TPL is one of the following:

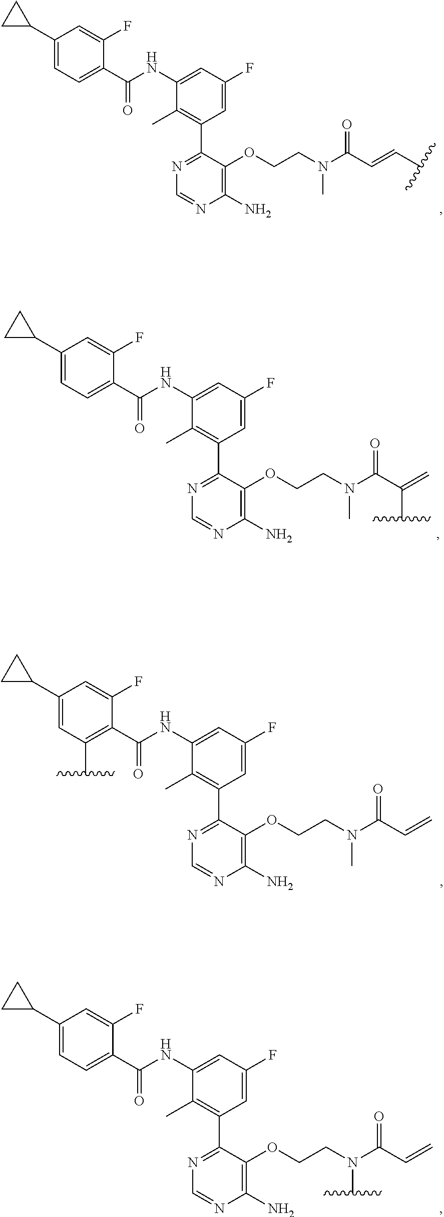 [SMILES: */C=C/C(=O)N(C)CCOc1c(N)ncnc1-c1cc(F)cc(NC(=O)c2ccc(C3CC3)cc2F)c1C ; *C(=C)C(=O)N(C)CCOc1c(N)ncnc1-c1cc(F)cc(NC(=O)c2ccc(C3CC3)cc2F)c1C ; *c1cc(C2CC2)cc(F)c1C(=O)Nc1cc(F)cc(-c2ncnc(N)c2OCCN(C)C(=O)C=C)c1C ; *N(CCOc1c(N)ncnc1-c1cc(F)cc(NC(=O)c2ccc(C3CC3)cc2F)c1C)C(=O)C=C]

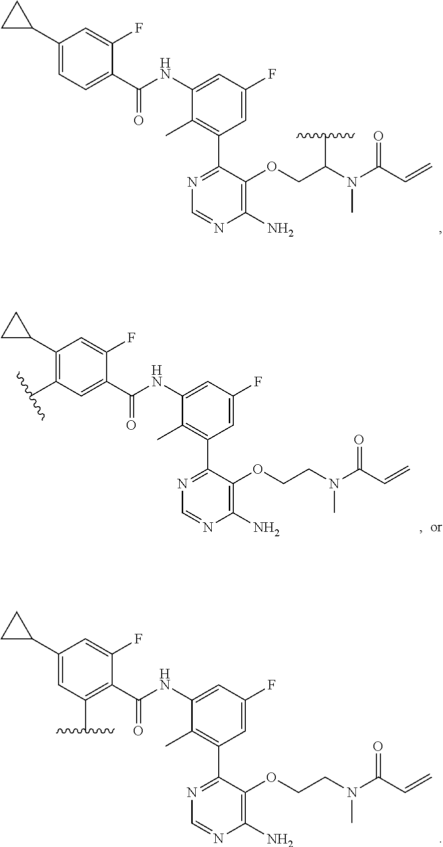 [SMILES: *C(COc1c(N)ncnc1-c1cc(F)cc(NC(=O)c2ccc(C3CC3)cc2F)c1C)N(C)C(=O)C=C ; *c1cc(C(=O)Nc2cc(F)cc(-c3ncnc(N)c3OCCN(C)C(=O)C=C)c2C)c(F)cc1C1CC1 ; *c1cc(C2CC2)cc(F)c1C(=O)Nc1cc(F)cc(-c2ncnc(N)c2OCCN(C)C(=O)C=C)c1C]

In certain embodiments, the TPL is a moiety that inhibits BTK. Compounds that inhibit BTK are reported in the literature, which include:

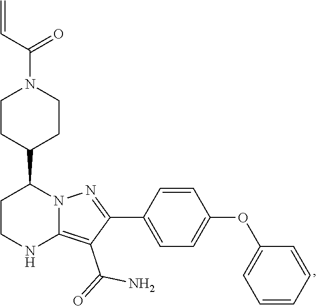 [SMILES: C=CC(=O)N1CCC([C@@H]2CCNc3c(C(N)=O)c(-c4ccc(Oc5ccccc5)cc4)nn32)CC1]

as described in Guo, Y. et al., in J Med Chem 2019, 62(17): 7923.

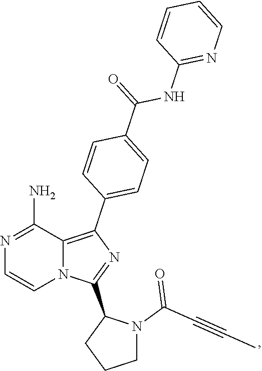 [SMILES: CC#CC(=O)N1CCC[C@H]1c1nc(-c2ccc(C(=O)Nc3ccccn3)cc2)c2c(N)nccn12]

as described in Hopper, M. Et al in J Pharmacol Exp Ther 2020, 372(3): 331.

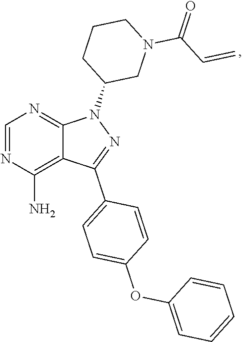 [SMILES: C=CC(=O)N1CCC[C@@H](n2nc(-c3ccc(Oc4ccccc4)cc3)c3c(N)ncnc32)C1]

as described in Honigbert, L. Et al in WO 2008/039218

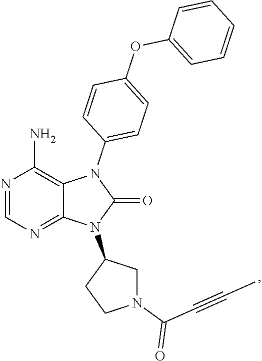 [SMILES: CC#CC(=O)N1CC[C@@H](n2c(=O)n(-c3ccc(Oc4ccccc4)cc3)c3c(N)ncnc32)C1]

as described in Yamamoto, S. Et al in WO 2011/152351

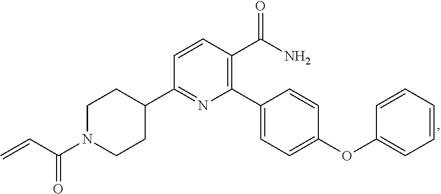 [SMILES: C=CC(=O)N1CCC(c2ccc(C(N)=O)c(-c3ccc(Oc4ccccc4)cc3)n2)CC1]

as described in Chen, X. Et al in WO 2015/048662

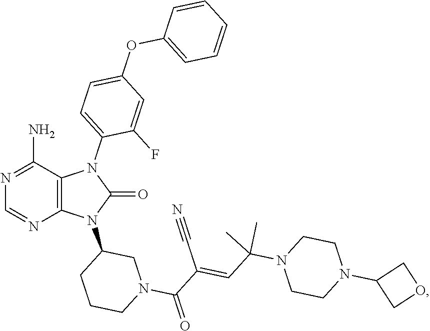 [SMILES: CC(C)(/C=C(\C#N)C(=O)N1CCC[C@@H](n2c(=O)n(-c3ccc(Oc4ccccc4)cc3F)c3c(N)ncnc32)C1)N1CCN(C2COC2)CC1]

as described in Owens, T. Et al in WO 2014/039899

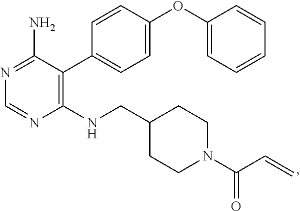 [SMILES: C=CC(=O)N1CCC(CNc2ncnc(N)c2-c2ccc(Oc3ccccc3)cc2)CC1]

as described in Caldwell, R. Et al in J Med Chem 2019, 62(17):

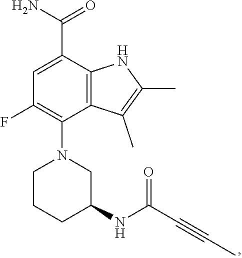 [SMILES: CC#CC(=O)N[C@H]1CCCN(c2c(F)cc(C(N)=O)c3[nH]c(C)c(C)c23)C1]

as described in Watterson, S. Et al in J Med Chem 2019, 62(7): 3228

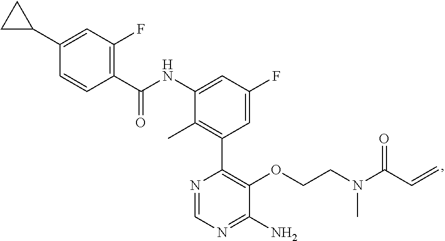 [SMILES: C=CC(=O)N(C)CCOc1c(N)ncnc1-c1cc(F)cc(NC(=O)c2ccc(C3CC3)cc2F)c1C]

as described in Angst, D. Et al in WO 2015/079417

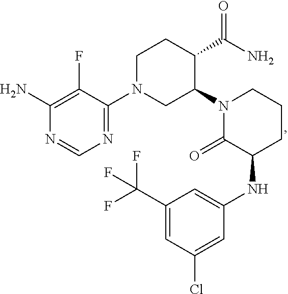 [SMILES: NC(=O)[C@H]1CCN(c2ncnc(N)c2F)C[C@@H]1N1CCC[C@@H](Nc2cc(Cl)cc(C(F)(F)F)c2)C1=O]

as described in Hopkins, B. Et al in WO 2013/185084.

In certain embodiments, the TPL is a radical of one of the above compounds, which is attached to L through a modifiable oxygen, nitrogen, or carbon atom.

In certain embodiments, the TPL has the following Formula:

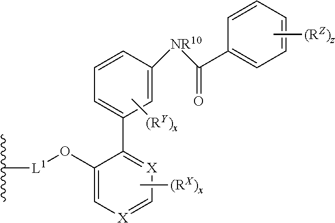

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
R¹⁰ is hydrogen or optionally substituted C₁₋₆ aliphatic group;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
L¹ is a covalent bond or a saturated or unsaturated, straight or branched, optionally substituted bivalent C₁₋₉ hydrocarbon chain, wherein 0-3 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
x, y, and z are each independently selected from 0, 1, 2, and 3.

In certain embodiments, L¹ is

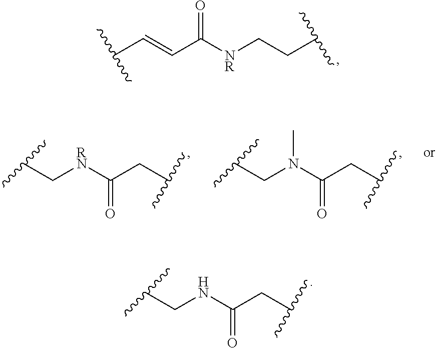

In some embodiments, the TPL has the formula:

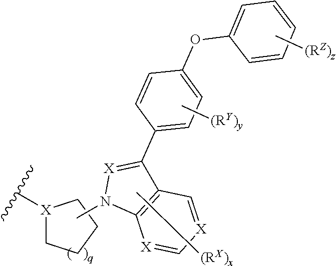

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
each of q, x, y, and z is independently 0, 1, 2, or 3.

In certain embodiments, the TPL has the formula:

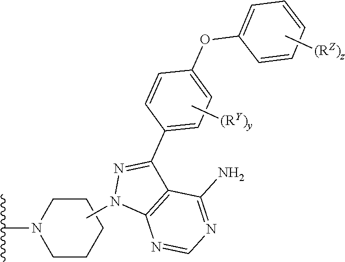

each instance of R^{Y} and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
each of y and z is independently 0, 1, 2, or 3.

### Moiety for EGFR

In certain embodiments, the TPL is a moiety that binds to EGFR. Exemplary compounds that bind to EGFR are reported in the literature, such as Osimertinib and mavelertinib. A radical of such compounds reported in the literature that bind EGFR are amenable for use in the present invention.

In certain embodiments, the TPL is one of the following:

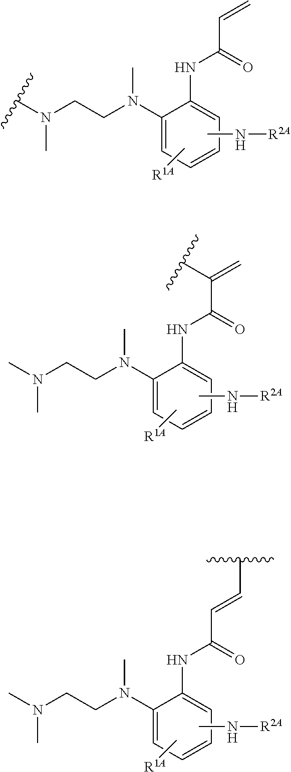

wherein:
R^{1A} is hydrogen, halo, hydroxyl, C₁₋₄ alkyl, C₁₋₄ alkoxyl, or N(R^{5A})(R^{6A});
R^{2A} is -(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl);
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

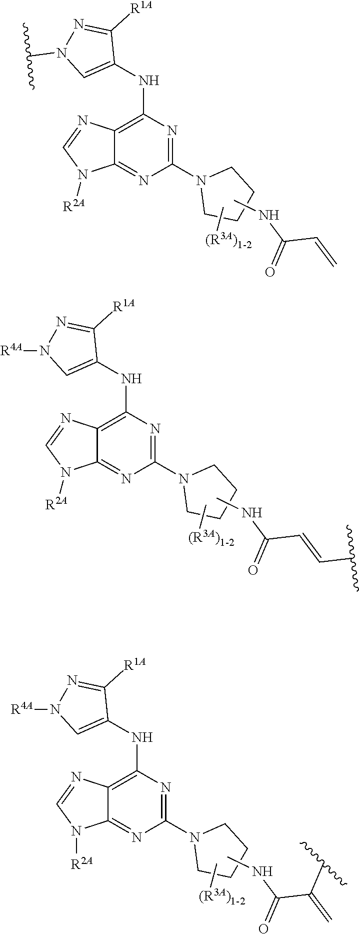

wherein:
R^{1A} is C₁₋₄ alkoxyl, C₁₋₄ alkyl, hydrogen, halo, or hydroxyl;
R^{2A} and R^{4A} are independently hydrogen or C₁₋₄ alkyl; anH
R^{3A} represents independently for each occurrence hydrogen, halo, hydroxyl, C₁₋₄ alkyl, or C₁₋₄ alkoxyl.

In certain embodiments, the TPL is one of the following:

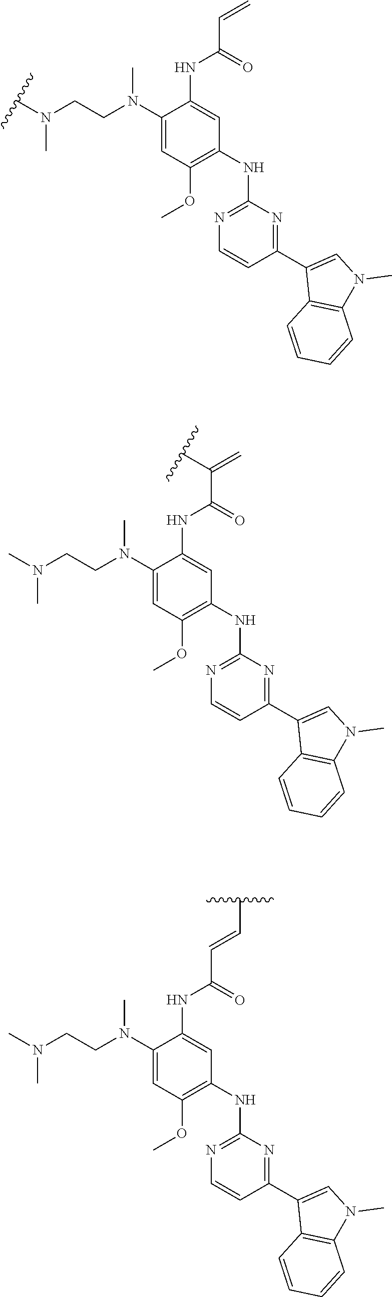

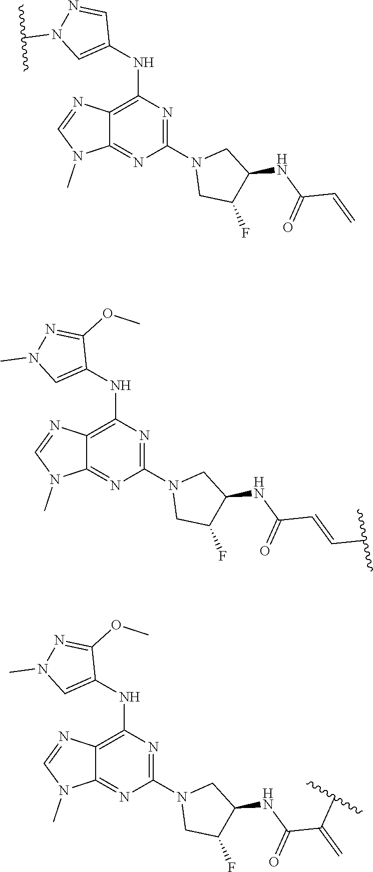

In certain embodiments, the TPL is a radical of

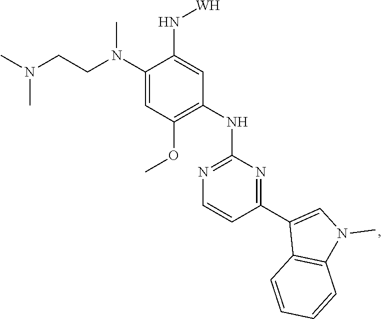

wherein WH is a group that reacts with EGFR to form a covalent linkage. In certain embodiments, the TPL is one of the following:

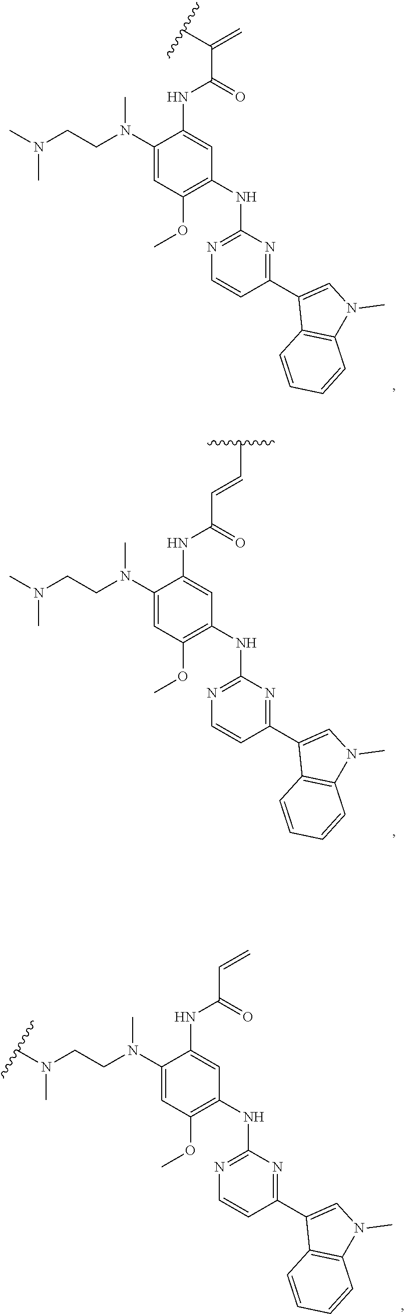

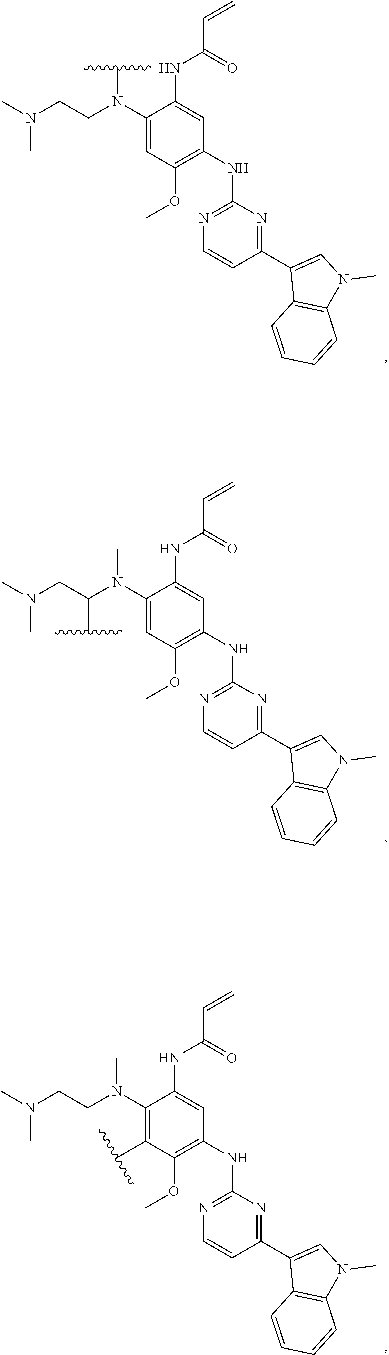

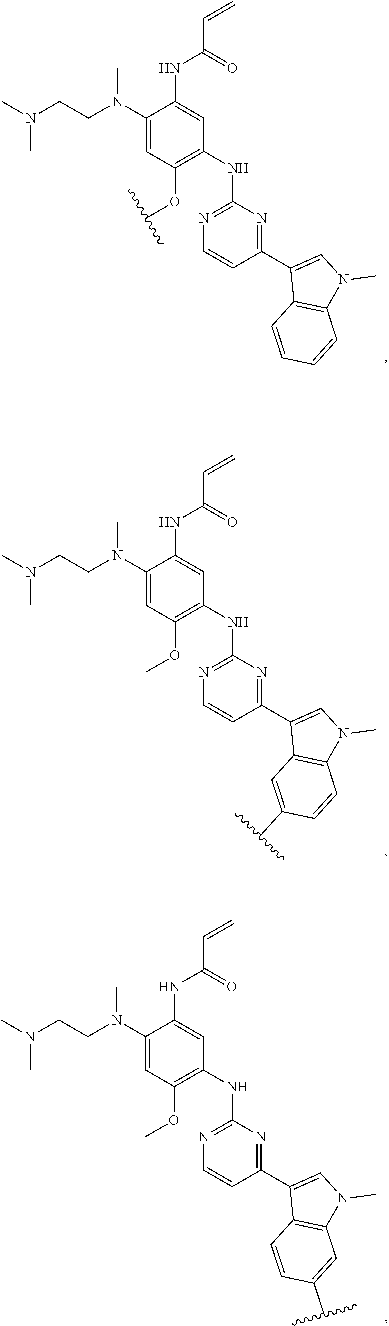

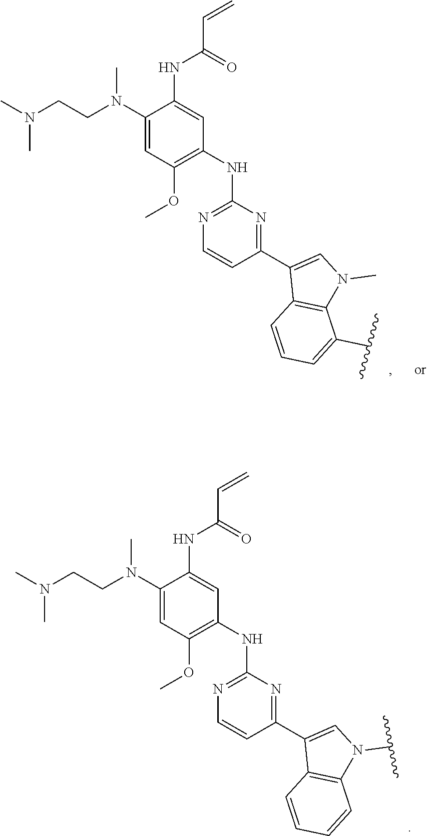

In certain embodiments, the TPL is a radical of

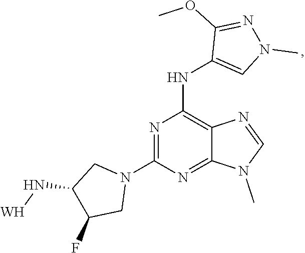

wherein WH is a group that reacts with EGFR to form a covalent linkage. In certain embodiments, the TPL is one of the following:

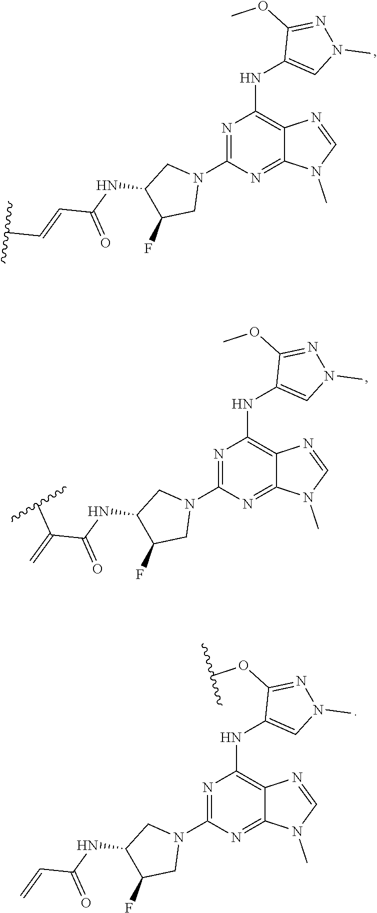

In certain embodiments, the TPL is a radical of

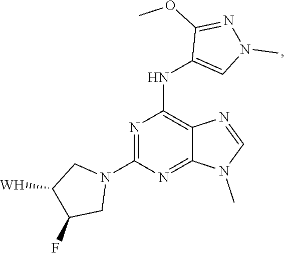

wherein WH is a group that reacts with EGFR to form a covalent linkage.

In certain embodiments, the TPL is a moiety that inhibits or binds to epidermal growth factor receptor (EGFR). Compounds that inhibit or bind to EGFR are reported in the literature, which include:

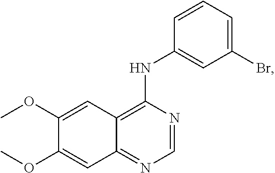 [SMILES: COc1cc2ncnc(Nc3cccc(Br)c3)c2cc1OC]

as described in Gangjee, A. et al., WO 2012/106522;

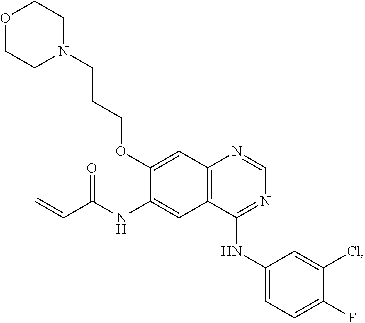 [SMILES: C=CC(=O)Nc1cc2c(Nc3ccc(F)c(Cl)c3)ncnc2cc1OCCCN1CCOCC1]

as described in Huang, Z. et al., WO 2012/027960;

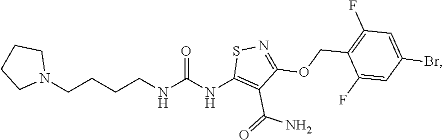 [SMILES: NC(=O)c1c(OCc2c(F)cc(Br)cc2F)nsc1NC(=O)NCCCCN1CCCC1]

as described in Bingaman, D. P. et al., WO 2014/152661;

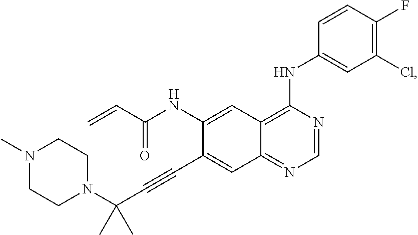 [SMILES: C=CC(=O)Nc1cc2c(Nc3ccc(F)c(Cl)c3)ncnc2cc1C#CC(C)(C)N1CCN(C)CC1]

as described in Kitano, Y. et al., WO 2002/066445;

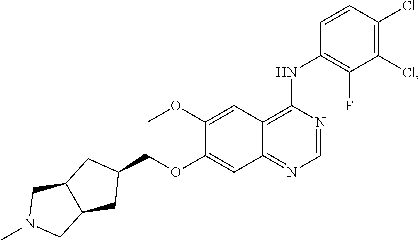 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

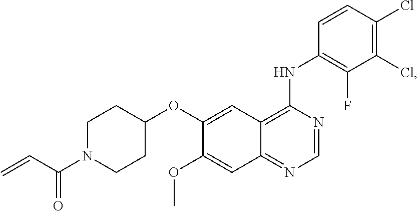 [SMILES: C=CC(=O)N1CCC(Oc2cc3c(Nc4ccc(Cl)c(Cl)c4F)ncnc3cc2OC)CC1]

as described in Lee, K.-O. et al., WO 2008/150118;

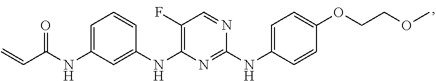 [SMILES: C=CC(=O)Nc1cccc(Nc2nc(Nc3ccc(OCCOC)cc3)ncc2F)c1]

as described in Kluge, A. F. et al., WO 2009/158571;

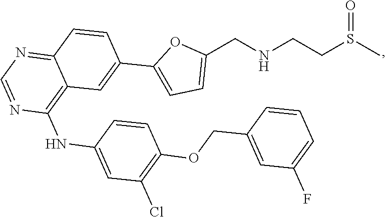 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

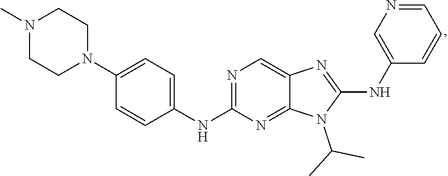 [SMILES: CC(C)n1c(Nc2cccnc2)nc2cnc(Nc3ccc(N4CCN(C)CC4)cc3)nc21]

as described in Li, D. Y. et al., WO 2014/135876;

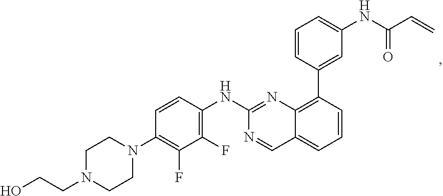 [SMILES: C=CC(=O)Nc1cccc(-c2cccc3cnc(Nc4ccc(N5CCN(CCO)CC5)c(F)c4F)nc23)c1]

as described in Qian, X. et al., WO

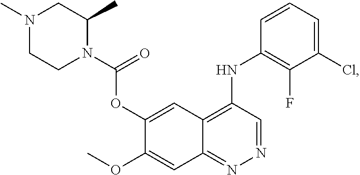 [SMILES: COc1cc2nncc(Nc3cccc(Cl)c3F)c2cc1OC(=O)N1CCN(C)C[C@H]1C]

2015/027222;

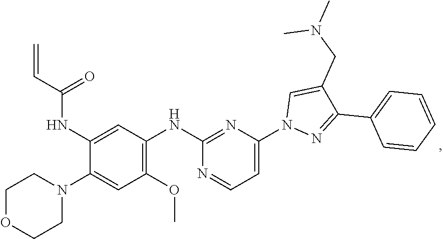 [SMILES: C=CC(=O)Nc1cc(Nc2nccc(-n3cc(CN(C)C)c(-c4ccccc4)n3)n2)c(OC)cc1N1CCOCC1]

as described in Suh, B.-C. et al., WO 2016/060443;

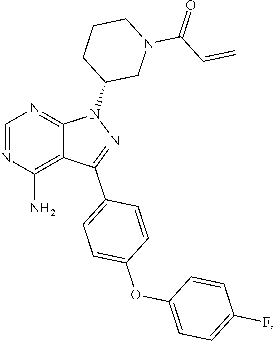 [SMILES: C=CC(=O)N1CCC[C@@H](n2nc(-c3ccc(Oc4ccc(F)cc4)cc3)c3c(N)ncnc32)C1]

as described in Zhang, D. et al., WO 2014/187319;

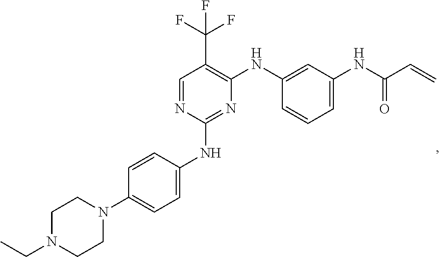 [SMILES: C=CC(=O)Nc1cccc(Nc2nc(Nc3ccc(N4CCN(CC)CC4)cc3)ncc2C(F)(F)F)c1]

as described in Zhang, D. et al., WO 2015/117547;

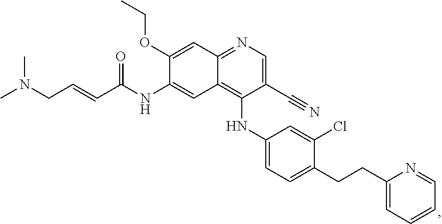 [SMILES: CCOc1cc2ncc(C#N)c(Nc3ccc(CCc4ccccn4)c(Cl)c3)c2cc1NC(=O)/C=C/CN(C)C]

as described in Wissner, A. et al., WO 2005/059678;

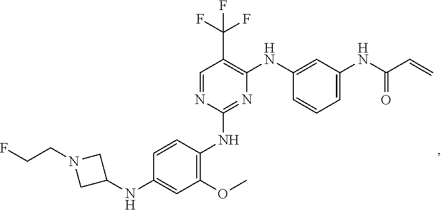 [SMILES: C=CC(=O)Nc1cccc(Nc2nc(Nc3ccc(NC4CN(CCF)C4)cc3OC)ncc2C(F)(F)F)c1]

as described in Lee, K. et al., WO 2012/064706.

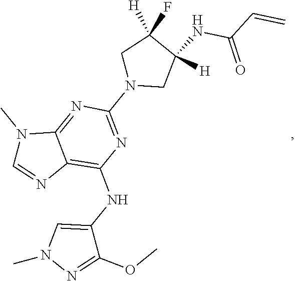 [SMILES: C=CC(=O)N[C@@H]1CN(c2nc(Nc3cn(C)nc3OC)c3ncn(C)c3n2)C[C@H]1F]

as described in Behenna, D. et al., WO 2015/075598.

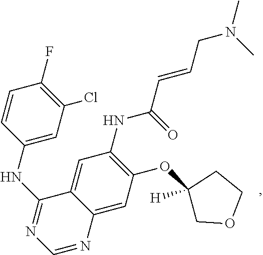 [SMILES: CN(C)C/C=C/C(=O)Nc1cc2c(Nc3ccc(F)c(Cl)c3)ncnc2cc1O[C@H]1CCOC1]

as described in Himmelsbach, F. et al., WO 2002/050043.

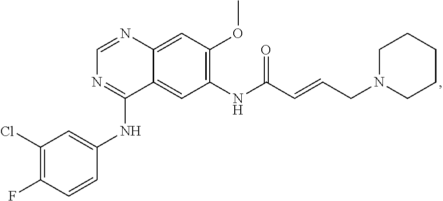 [SMILES: COc1cc2ncnc(Nc3ccc(F)c(Cl)c3)c2cc1NC(=O)/C=C/CN1CCCCC1]

as described in Fakhoury, S. et al., WO 2005/107758.

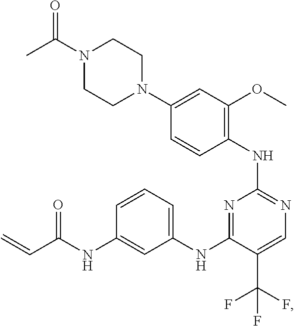 [SMILES: C=CC(=O)Nc1cccc(Nc2nc(Nc3ccc(N4CCN(C(C)=O)CC4)cc3OC)ncc2C(F)(F)F)c1]

as described in Lee, K. et al., WO 2012/061299.

In certain embodiments, the TPL is a radical of one of the above compounds, which is attached to L through a modifiable oxygen, nitrogen, or carbon atom.

In certain embodiments, the TPL has the formula:

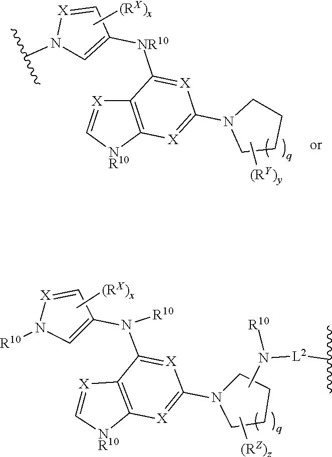

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
R¹⁰ is hydrogen or optionally substituted C₁₋₆ aliphatic group;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
L² is a covalent bond or a saturated or unsaturated, straight or branched, optionally substituted bivalent C₁₋₇ hydrocarbon chain, wherein 0-3 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
q, x, y, and z are each independently selected from 0, 1, 2, and or 3.

In certain embodiments, the TPL has the formula:

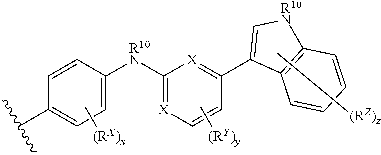

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
each R¹⁰ is independently hydrogen or optionally substituted C₁₋₆ aliphatic group;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
x, y, and z are each independently selected from 0, 1, 2, and or 3.

In some embodiments, the TPL has the formula:

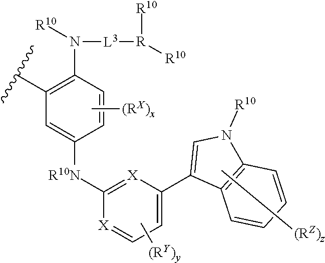

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
each R¹⁰ is independently hydrogen or optionally substituted C₁₋₆ aliphatic group;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
x, y, and z are each independently selected from 0, 1, 2, and 3.

### Moiety for AR

In certain embodiments, the TPL is a moiety that binds to androgen receptor (AR) protein. Exemplary compounds that bind to AR are reported in the literature, such as TMBC and 5N-bicalutamide. A radical of such compounds reported in the literature that bind AR are amenable for use in the present invention.

In certain embodiments, the TPL is one of the following:

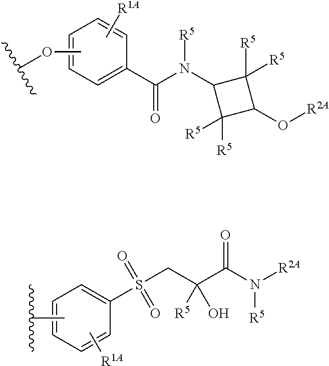

wherein:
R^{1A} is hydrogen, halo, hydroxyl, C₁₋₄ alkyl, C₁₋₄ alkoxyl, or —N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one following:

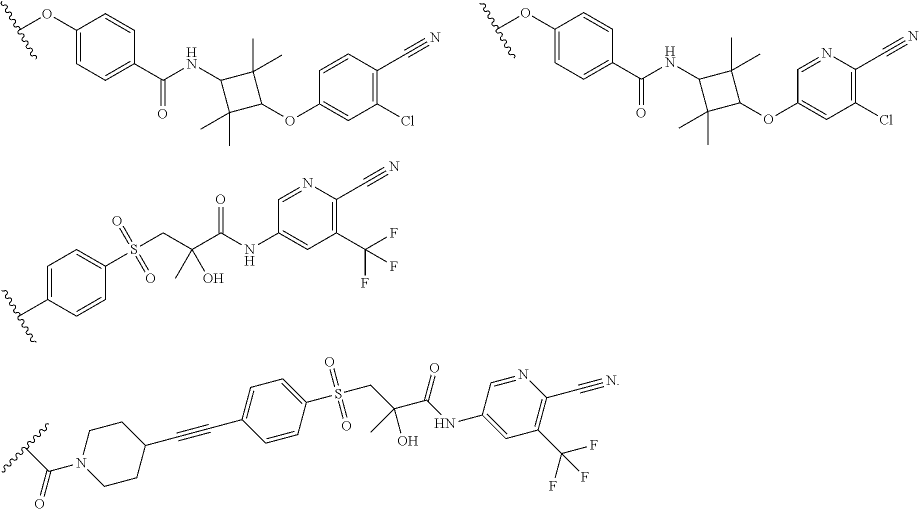 [SMILES: *Oc1ccc(C(=O)NC2C(C)(C)C(Oc3ccc(C#N)c(Cl)c3)C2(C)C)cc1 ; *Oc1ccc(C(=O)NC2C(C)(C)C(Oc3cnc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1]

In certain embodiments, the TPL is

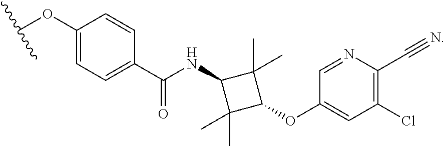 [SMILES: *Oc1ccc(C(=O)N[C@H]2C(C)(C)[C@H](Oc3cnc(C#N)c(Cl)c3)C2(C)C)cc1]

In certain embodiments, the TPL is

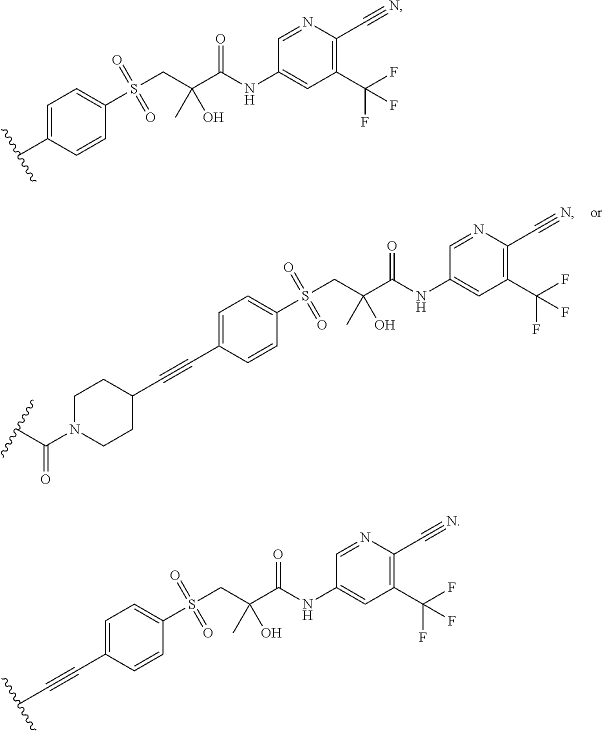 [SMILES: *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1 ; *C#Cc1ccc(S(=O)(=O)CC(C)(O)C(=O)Nc2cnc(C#N)c(C(F)(F)F)c2)cc1]

In certain embodiments, the TPL

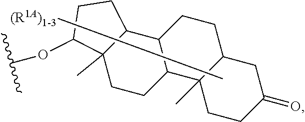

wherein R^{1A} represents independently for each occurrence hydrogen, halo, hydroxyl, C₁₋₄ alkyl, or C₁₋₄ alkoxyl.

In certain embodiments, the TPL is

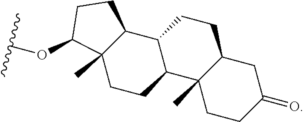 [SMILES: *O[C@H]1CC[C@H]2[C@@H]3CC[C@H]4CC(=O)CC[C@]4(C)[C@H]3CC[C@]12C]

In certain embodiments, TPL is a moiety that is an agonist of the androgen receptor (AR) protein. Compounds that are agonists of the AR are reported in the literature, which include:

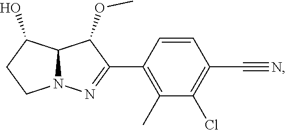 [SMILES: CO[C@@H]1C(c2ccc(C#N)c(Cl)c2C)=NN2CC[C@H](O)[C@@H]12]

as described in Ullrich, T. et al., in WO 2013/014627.

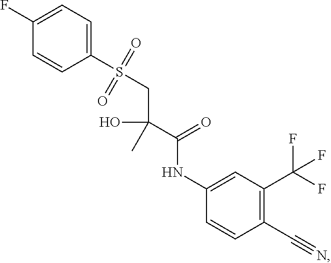 [SMILES: CC(O)(CS(=O)(=O)c1ccc(F)cc1)C(=O)Nc1ccc(C#N)c(C(F)(F)F)c1]

as described in Yoshino, H. et al., in Bioorg Med Chem 2010, 18(23): 8150.

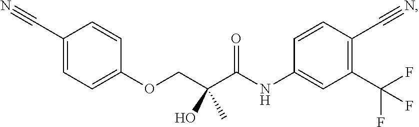 [SMILES: C[C@](O)(COc1ccc(C#N)cc1)C(=O)Nc1ccc(C#N)c(C(F)(F)F)c1]

as described in Steiner, M. et al., in US 2016/128968.

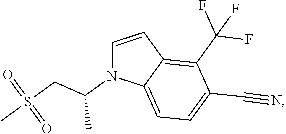 [SMILES: C[C@H](CS(C)(=O)=O)n1ccc2c(C(F)(F)F)c(C#N)ccc21]

as described in Cadilla, R. et al., in US 2019/127326.

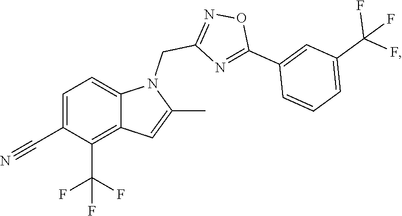 [SMILES: Cc1cc2c(C(F)(F)F)c(C#N)ccc2n1Cc1noc(-c2cccc(C(F)(F)F)c2)n1]

as described in Turnbull, P. et al., in 249th Am Chem Soc (ACS) Natl Meet⋅2015-03-22/2015-03-26⋅Denver, United States Abst MEDI 247.

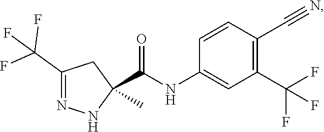 [SMILES: C[C@@]1(C(=O)Nc2ccc(C#N)c(C(F)(F)F)c2)CC(C(F)(F)F)=NN1]

as described in Allan, G. et al., in Endocrine 2007, 32(1): 41.

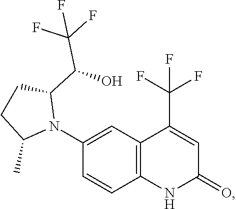 [SMILES: C[C@@H]1CC[C@H]([C@@H](O)C(F)(F)F)N1c1ccc2[nH]c(=O)cc(C(F)(F)F)c2c1]

as described in Martinborough, E. et al., in J Med Chem 2007, 50(21): 5049.

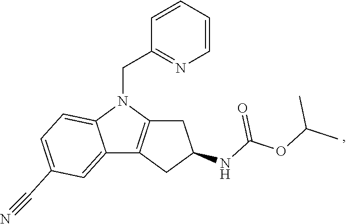 [SMILES: CC(C)OC(=O)N[C@H]1Cc2c(n(Cc3ccccn3)c3ccc(C#N)cc23)C1]

as described in Benson, C. et al., in WO 2016/040234.

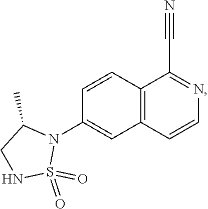 [SMILES: C[C@H]1CNS(=O)(=O)N1c1ccc2c(C#N)nccc2c1]

as described in Chekler, E. et al., in 246th Am Chem Soc (ACS) Natl Meet⋅2013-09-08/2013-09-12⋅Indianapolis, United States Abst MEDI 30.

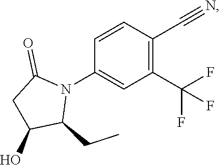 [SMILES: CC[C@H]1[C@@H](O)CC(=O)N1c1ccc(C#N)c(C(F)(F)F)c1]

as described in Aikawa, K. et al., in Bioorg Med Chem 2017, 25(13): 3330.

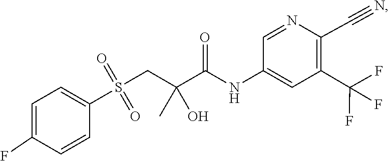 [SMILES: CC(O)(CS(=O)(=O)c1ccc(F)cc1)C(=O)Nc1cnc(C#N)c(C(F)(F)F)c1]

as described in Cortez F. et al., in ACS Chem. Biol. 2017, 12(12): 2934.

In certain embodiments, the TPL is a radical of one of the above compounds, which is attached to L through a modifiable oxygen, nitrogen, or carbon atom.

In certain embodiments, the TPL is a moiety that is an antagonist of the androgen receptor (AR). Compounds that are an antagonist of the AR are reported in the literature, which include:

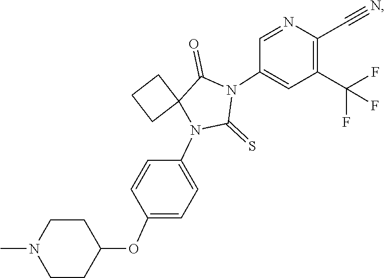 [SMILES: CN1CCC(Oc2ccc(N3C(=S)N(c4cnc(C#N)c(C(F)(F)F)c4)C(=O)C34CCC4)cc2)CC1]

as described in Bignan, G. et al., in WO 2018/009694.

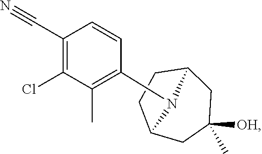 [SMILES: Cc1c(N2[C@@H]3CC[C@H]2C[C@@](C)(O)C3)ccc(C#N)c1Cl]

as described in Sunden, H. et al., in J Med Chem 2015, 58(3): 1569.

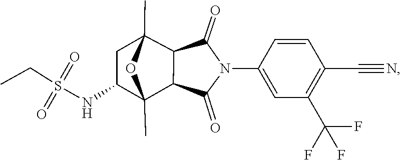 [SMILES: CCS(=O)(=O)N[C@@H]1C[C@@]2(C)O[C@]1(C)[C@@H]1C(=O)N(c3ccc(C#N)c(C(F)(F)F)c3)C(=O)[C@@H]12]

as described in Balog, A. et al., in ACS Med Chem Lett 2015, 6(8): 908.

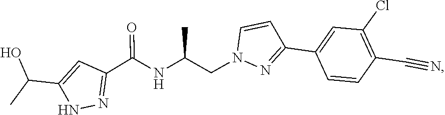 [SMILES: CC(O)c1cc(C(=O)N[C@@H](C)Cn2ccc(-c3ccc(C#N)c(Cl)c3)n2)n[nH]1]

as described in Sugawara, T. et al., in Int J Cancer 2019, 145(5): 1382.

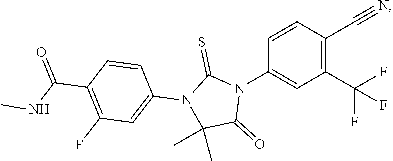 [SMILES: CNC(=O)c1ccc(N2C(=S)N(c3ccc(C#N)c(C(F)(F)F)c3)C(=O)C2(C)C)cc1F]

as described in Sawyers, C. et al., in WO 2006/124118

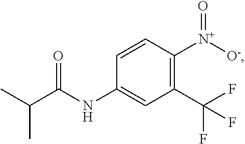

as described in Rizner, T. et al., in Steroids 2011, 76(6): 607.

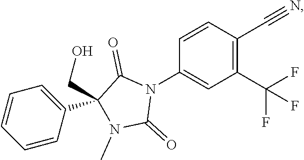 [SMILES: CN1C(=O)N(c2ccc(C#N)c(C(F)(F)F)c2)C(=O)[C@@]1(CO)c1ccccc1]

as described in Sabchareon, A. et al., in J Med Chem 2012, 55(19): 8236.

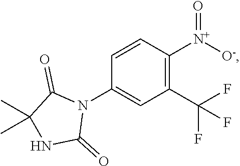

as described in Schlienger, N. et al., in J Med Chem 2009, 52(22): 7186.

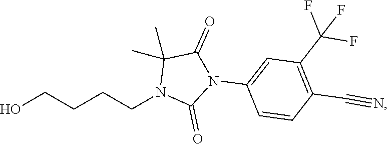 [SMILES: CC1(C)C(=O)N(c2ccc(C#N)c(C(F)(F)F)c2)C(=O)N1CCCCO]

as described in Huang, T. et al., in J Med Chem 2010, 53(11): 4422.

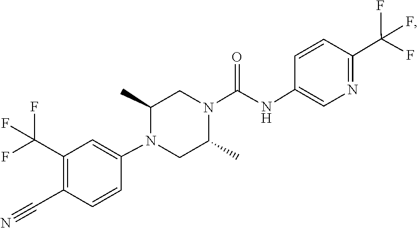 [SMILES: C[C@@H]1CN(c2ccc(C#N)c(C(F)(F)F)c2)[C@@H](C)CN1C(=O)Nc1ccc(C(F)(F)F)nc1]

as described in Kinoyama, I. et al., in J Med Chem 2006, 49(2): 716.

In certain embodiments, the TPL is a radical of one of the above compounds, which is attached to L through a modifiable oxygen, nitrogen, or carbon atom.

### Moiety for IDH1

In certain embodiments, the TPL is a moiety that binds to IDH1. Exemplary compounds that bind to IDH1 are reported in the literature, such as LY3410738. A radical of such compounds reported in the literature that bind IDH1 are amenable for use in the present invention.

In certain embodiments, the TPL is a radical of

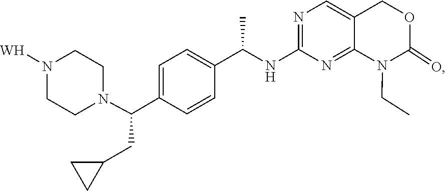

wherein WH is a group that reacts with IDH1 to form a covalent linkage. In certain embodiments, the TPL is one of the following:

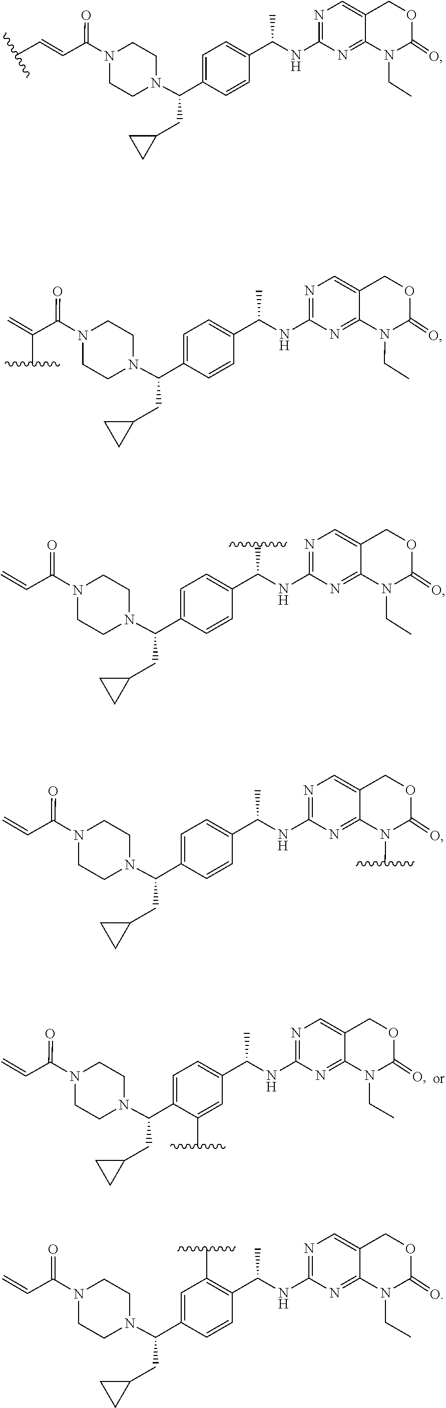 [SMILES: */C=C/C(=O)N1CCN([C@@H](CC2CC2)c2ccc([C@H](C)Nc3ncc4c(n3)N(CC)C(=O)OC4)cc2)CC1 ; *C(=C)C(=O)N1CCN([C@@H](CC2CC2)c2ccc([C@H](C)Nc3ncc4c(n3)N(CC)C(=O)OC4)cc2)CC1 ; *[C@H](Nc1ncc2c(n1)N(CC)C(=O)OC2)c1ccc([C@H](CC2CC2)N2CCN(C(=O)C=C)CC2)cc1 ; *N1C(=O)OCc2cnc(N[C@@H](C)c3ccc([C@H](CC4CC4)N4CCN(C(=O)C=C)CC4)cc3)nc21 ; *c1cc([C@H](C)Nc2ncc3c(n2)N(CC)C(=O)OC3)ccc1[C@H](CC1CC1)N1CCN(C(=O)C=C)CC1 ; *c1cc([C@H](CC2CC2)N2CCN(C(=O)C=C)CC2)ccc1[C@H](C)Nc1ncc2c(n1)N(CC)C(=O)OC2]

### Moiety for KRas

In certain embodiments, the TPL is a moiety that binds to KRas. Exemplary compounds that bind to KRas are reported in the literature, such as MRTX849 and AMG510. A radical of such compounds reported in the literature that bind KRas are amenable for use in the present invention.

In certain embodiments, the TPL is one of the following:

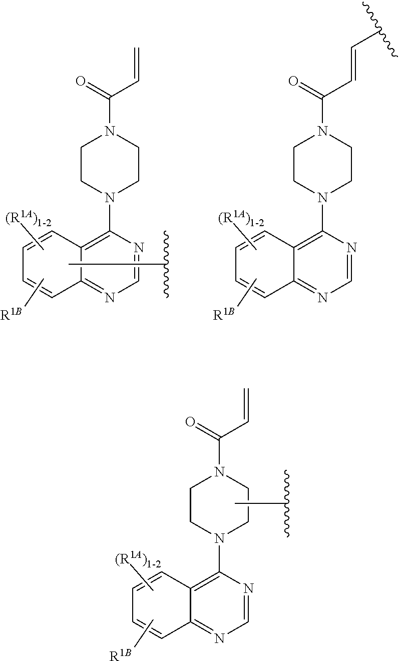

wherein:
R^{1A} represents independently for each occurrence hydrogen, halo, hydroxyl, C₁₋₄ alkyl, or C₁₋₄ alkoxyl; and
R^{1B} is C₆₋₁₂ aryl optionally substituted by 1, 2, or 3 substituents independently selected from halo, hydroxyl, C₁₋₄ alkyl, or C₁₋₄ alkoxyl.

In certain embodiments, the TPL is one of the following:

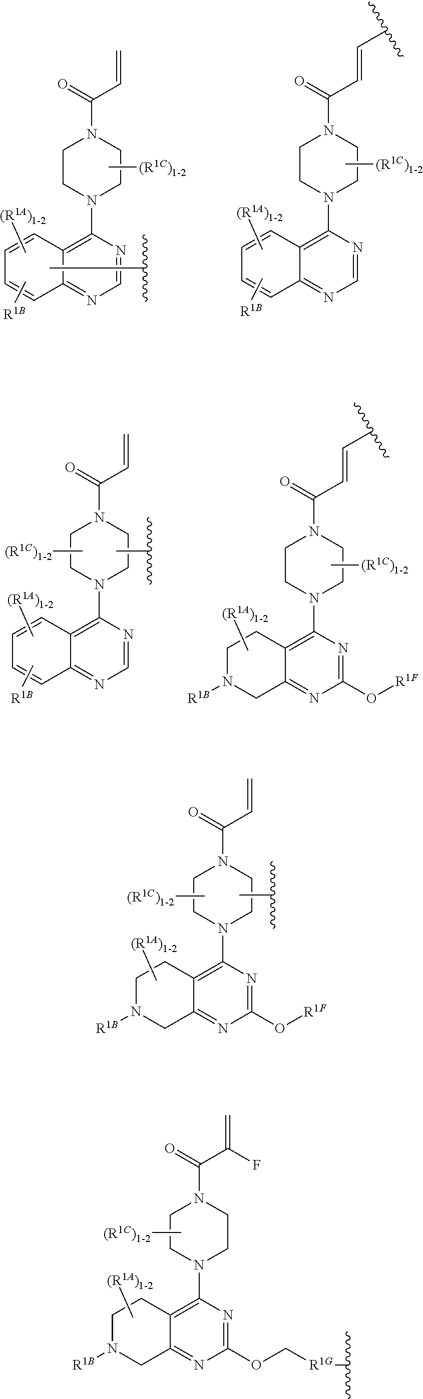

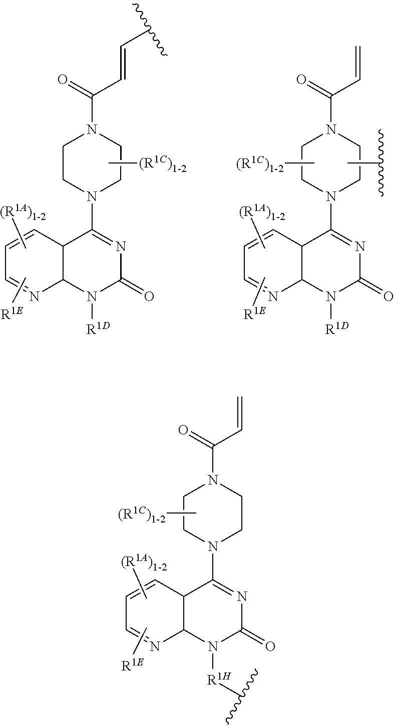

wherein:
R^{1A} represents independently for each occurrence hydrogen, halo, hydroxyl, C₁₋₄ alkyl, or C₁₋₄ alkoxyl;
R^{1B} is C₆₋₁₂ aryl optionally substituted by 1, 2, or 3 substituents independently selected from halo, hydroxyl, C₁₋₄ alkyl, or C₁₋₄ alkoxyl;
R^{1C} represents independently for each occurrence (C₁₋₄ alkylene)-CN, or C₁₋₄ alkyl;
R^{1D} are R^{1E} are independently phenyl or 5-6 membered heteroaryl containing 1, 2, or 3 heteroatoms selected from oxygen, nitrogen, or sulfur, wherein the phenyl or heteroaryl is substituted with 0, 1, 2, or 3 substituents independently selected from the group consisting of halo, hydroxyl, C₁₋₄ alkyl, or C₁₋₄ alkoxyl;
R^{1F} is —(C₁₋₄ alkylene)-(3-7 membered heterocyclyl containing 1, 2, or 3 heteroatoms independently selected from oxygen, nitrogen, or sulfur, wherein the heterocyclyl is substituted with 0, 1, 2, or 3 substituents independently selected from the group consisting of halo, hydroxyl, C₁₋₄ alkyl, or C₁₋₄ alkoxyl);
R^{1G} is 3-7 membered heterocyclylene containing 1, 2, or 3 heteroatoms independently selected from heteroatoms independently selected from oxygen, nitrogen, or sulfur, wherein the heterocyclylene is substituted with 0, 1, 2, or 3 substituents independently selected from the group consisting of halo, hydroxyl, C₁₋₄ alkyl, or C₁₋₄ alkoxyl); and
R^{1H} is phenylene or 5-6 membered heteroarylene containing 1, 2, or 3 heteroatoms selected from oxygen, nitrogen, or sulfur, wherein the phenylene or heteroarylene is substituted with 0, 1, 2, or 3 substituents independently selected from the group consisting of halo, hydroxyl, C₁₋₄ alkyl, or ₁₋₄ alkoxyl.

In certain embodiments, the TPL is one of the following:

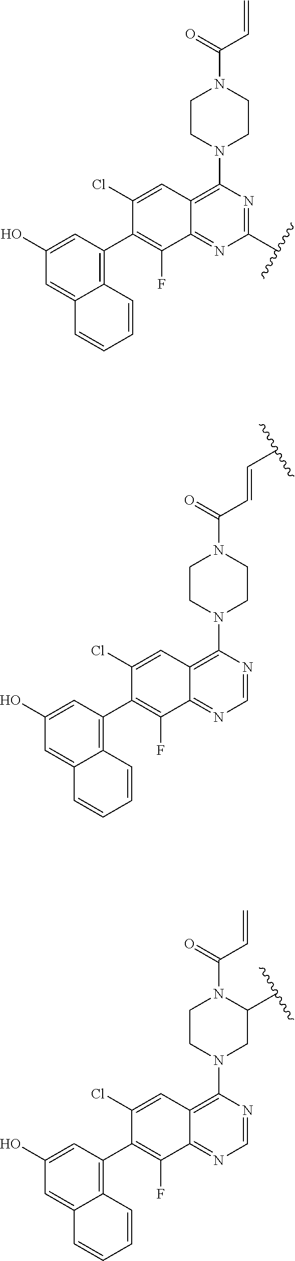 [SMILES: *c1nc(N2CCN(C(=O)C=C)CC2)c2cc(Cl)c(-c3cc(O)cc4ccccc34)c(F)c2n1 ; */C=C/C(=O)N1CCN(c2ncnc3c(F)c(-c4cc(O)cc5ccccc45)c(Cl)cc23)CC1 ; *C1CN(c2ncnc3c(F)c(-c4cc(O)cc5ccccc45)c(Cl)cc23)CCN1C(=O)C=C]

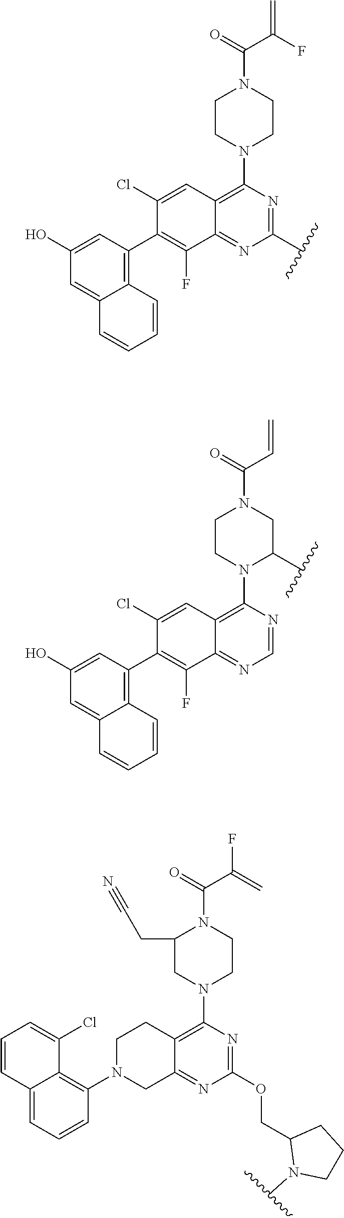 [SMILES: *c1nc(N2CCN(C(=O)C(=C)F)CC2)c2cc(Cl)c(-c3cc(O)cc4ccccc34)c(F)c2n1 ; *C1CN(C(=O)C=C)CCN1c1ncnc2c(F)c(-c3cc(O)cc4ccccc34)c(Cl)cc12 ; *N1CCCC1COc1nc2c(c(N3CCN(C(=O)C(=C)F)C(CC#N)C3)n1)CCN(c1cccc3cccc(Cl)c13)C2]

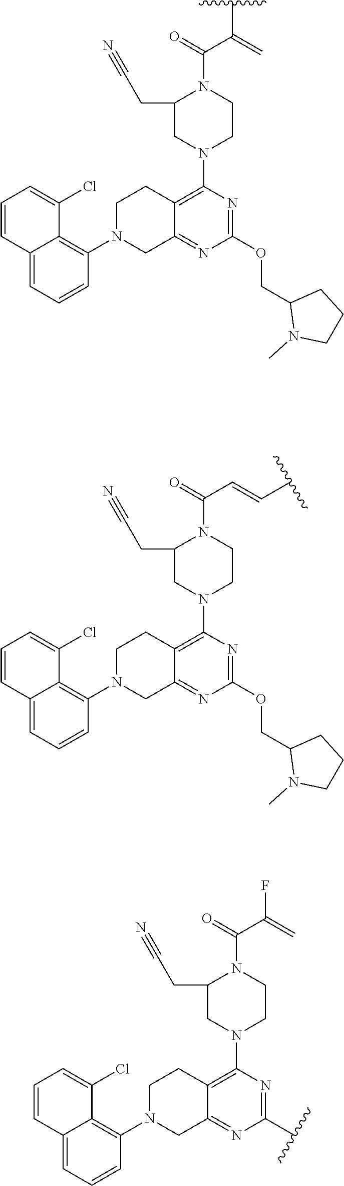 [SMILES: *C(=C)C(=O)N1CCN(c2nc(OCC3CCCN3C)nc3c2CCN(c2cccc4cccc(Cl)c24)C3)CC1CC#N ; */C=C/C(=O)N1CCN(c2nc(OCC3CCCN3C)nc3c2CCN(c2cccc4cccc(Cl)c24)C3)CC1CC#N ; *c1nc2c(c(N3CCN(C(=O)C(=C)F)C(CC#N)C3)n1)CCN(c1cccc3cccc(Cl)c13)C2]

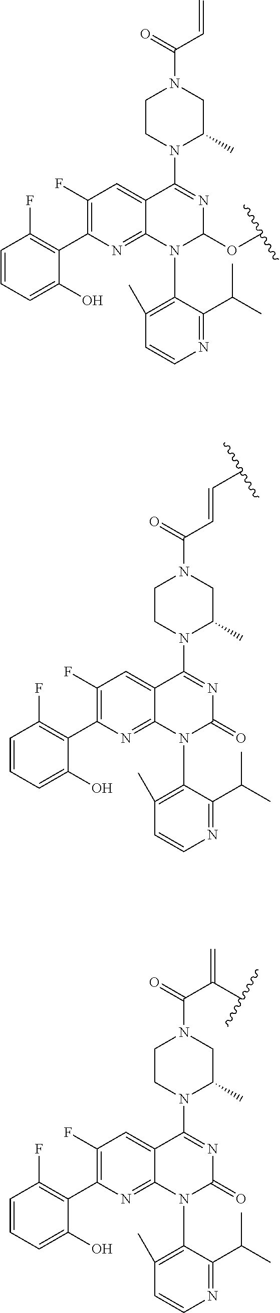 [SMILES: *OC1N=C(N2CCN(C(=O)C=C)C[C@@H]2C)c2cc(F)c(-c3c(O)cccc3F)nc2N1c1c(C)ccnc1C(C)C ; */C=C/C(=O)N1CCN(c2nc(=O)n(-c3c(C)ccnc3C(C)C)c3nc(-c4c(O)cccc4F)c(F)cc23)[C@@H](C)C1 ; *C(=C)C(=O)N1CCN(c2nc(=O)n(-c3c(C)ccnc3C(C)C)c3nc(-c4c(O)cccc4F)c(F)cc23)[C@@H](C)C1]

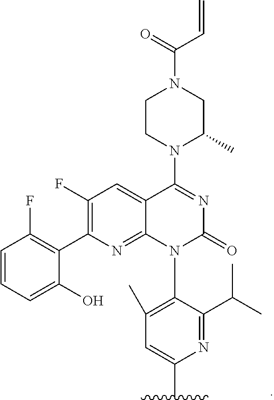 [SMILES: *c1cc(C)c(-n2c(=O)nc(N3CCN(C(=O)C=C)C[C@@H]3C)c3cc(F)c(-c4c(O)cccc4F)nc32)c(C(C)C)n1]

In certain embodiments, the TPL is one of the following:

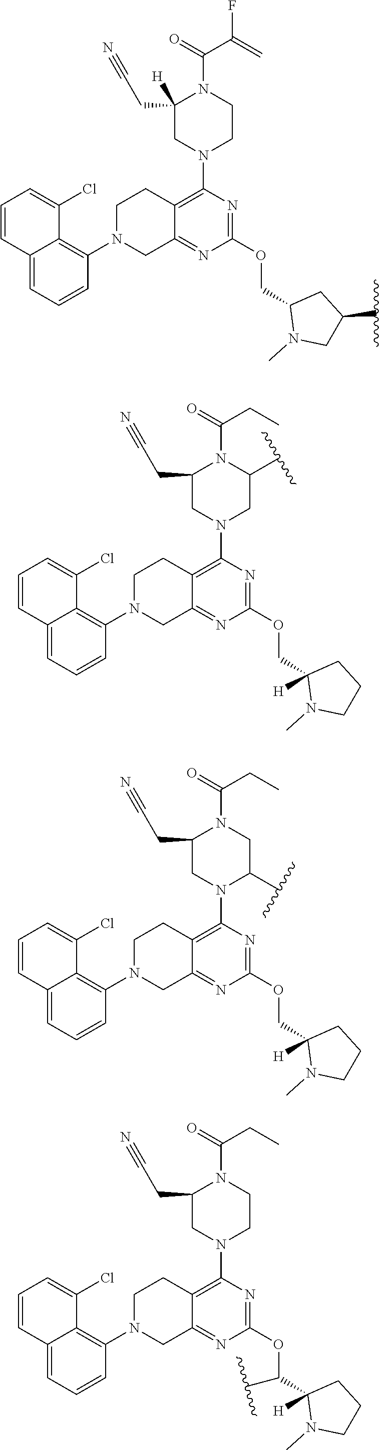 [SMILES: *[C@@H]1C[C@@H](COc2nc3c(c(N4CCN(C(=O)C(=C)F)[C@@H](CC#N)C4)n2)CCN(c2cccc4cccc(Cl)c24)C3)N(C)C1 ; *C1CN(c2nc(OC[C@@H]3CCCN3C)nc3c2CCN(c2cccc4cccc(Cl)c24)C3)C[C@@H](CC#N)N1C(=O)CC ; *C1CN(C(=O)CC)[C@H](CC#N)CN1c1nc(OC[C@@H]2CCCN2C)nc2c1CCN(c1cccc3cccc(Cl)c13)C2 ; *C(Oc1nc2c(c(N3CCN(C(=O)CC)[C@H](CC#N)C3)n1)CCN(c1cccc3cccc(Cl)c13)C2)[C@@H]1CCCN1C]

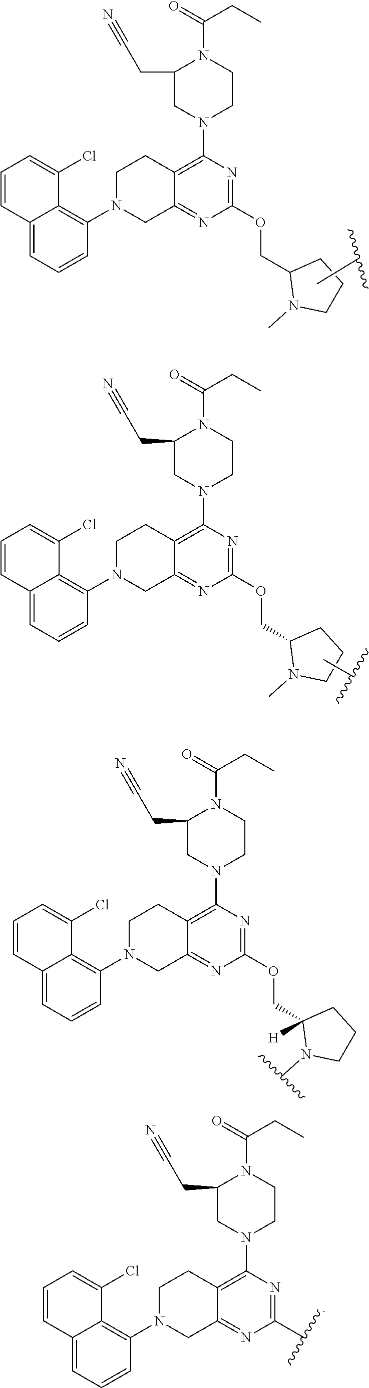

In certain embodiments, the TPL is a radical

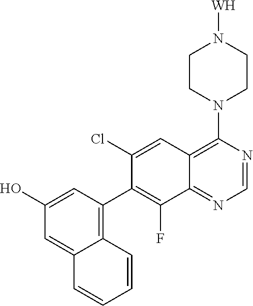

wherein WH is a group that reacts with KRas to form a covalent linkage. In certain embodiments, the TPL is one of the following:

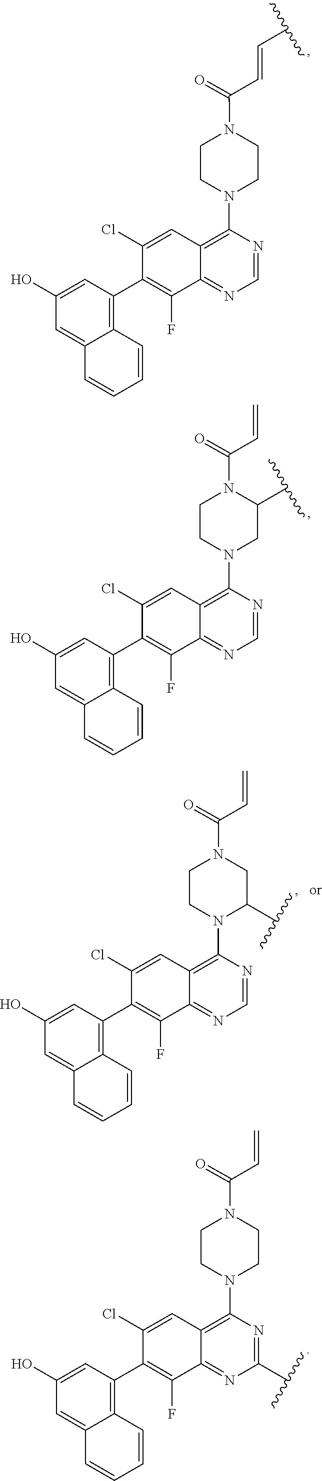 [SMILES: */C=C/C(=O)N1CCN(c2ncnc3c(F)c(-c4cc(O)cc5ccccc45)c(Cl)cc23)CC1 ; *C1CN(c2ncnc3c(F)c(-c4cc(O)cc5ccccc45)c(Cl)cc23)CCN1C(=O)C=C ; *C1CN(C(=O)C=C)CCN1c1ncnc2c(F)c(-c3cc(O)cc4ccccc34)c(Cl)cc12 ; *c1nc(N2CCN(C(=O)C=C)CC2)c2cc(Cl)c(-c3cc(O)cc4ccccc34)c(F)c2n1]

In certain embodiments, the TPL is a radical of

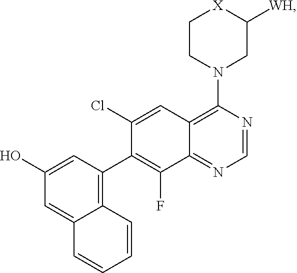

wherein X is NH, NR^{a}, CH₂, CHR^{a}, or C(R^{a})₂; R^{a} is C₁₋₆ alkyl, C₂₋₆ alkenyl, amido, amino, aminoalkyl, or C₁₋₆ alkoxy, and WH is a group that reacts with KRas to form a covalent linkage.

In certain embodiments, the TPL is a radical of

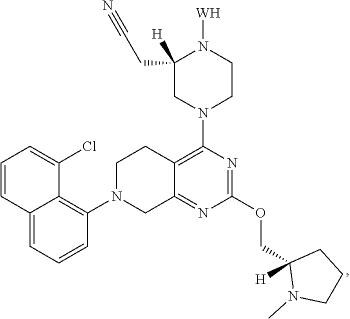

wherein WH is a group that reacts with KRas to form a covalent linkage.

In certain embodiments, the TPL is a radical of

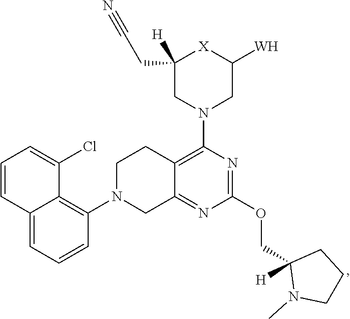

wherein WH is a group that reacts with KRas to form a covalent linkage; X is NH, NR^{a}, CH₂, CHR^{a}, or C(R^{a})₂; and R^{a} is C₁₋₆ alkyl, C₂₋₆ alkenyl, amido, amino, aminoalkyl, or C₁₋₆ alkoxy.

In certain embodiments, the TPL is

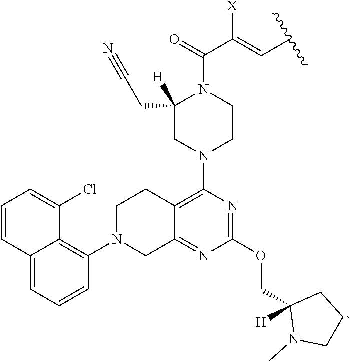

where X is hydrogen or halo.

In certain embodiments, the TPL is one of the following:

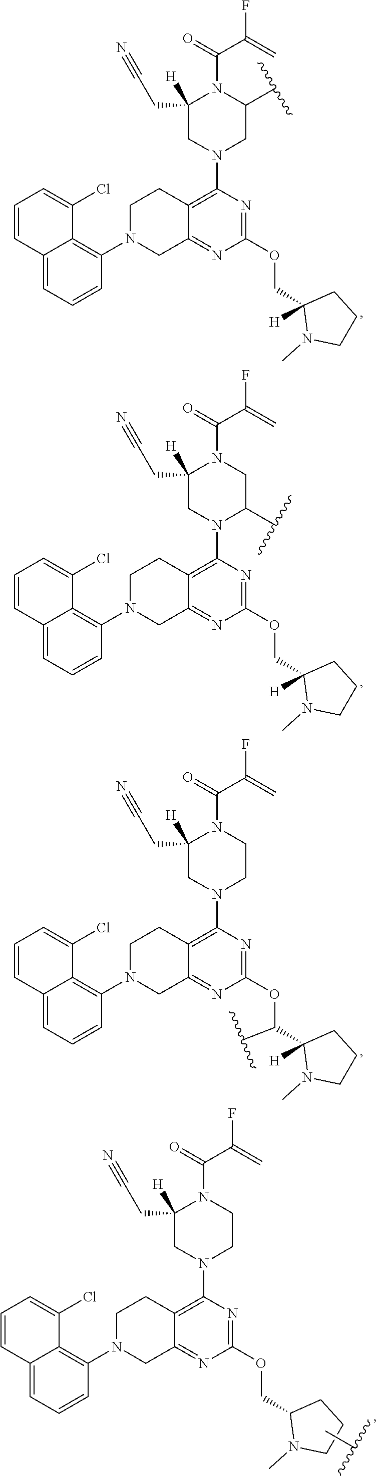

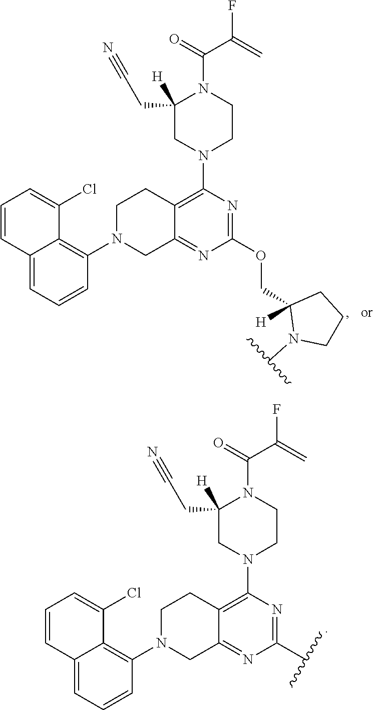 [SMILES: *N1CCC[C@H]1COc1nc2c(c(N3CCN(C(=O)C(=C)F)[C@@H](CC#N)C3)n1)CCN(c1cccc3cccc(Cl)c13)C2 ; *c1nc2c(c(N3CCN(C(=O)C(=C)F)[C@@H](CC#N)C3)n1)CCN(c1cccc3cccc(Cl)c13)C2]

In certain embodiments, the TPL is one of the following:

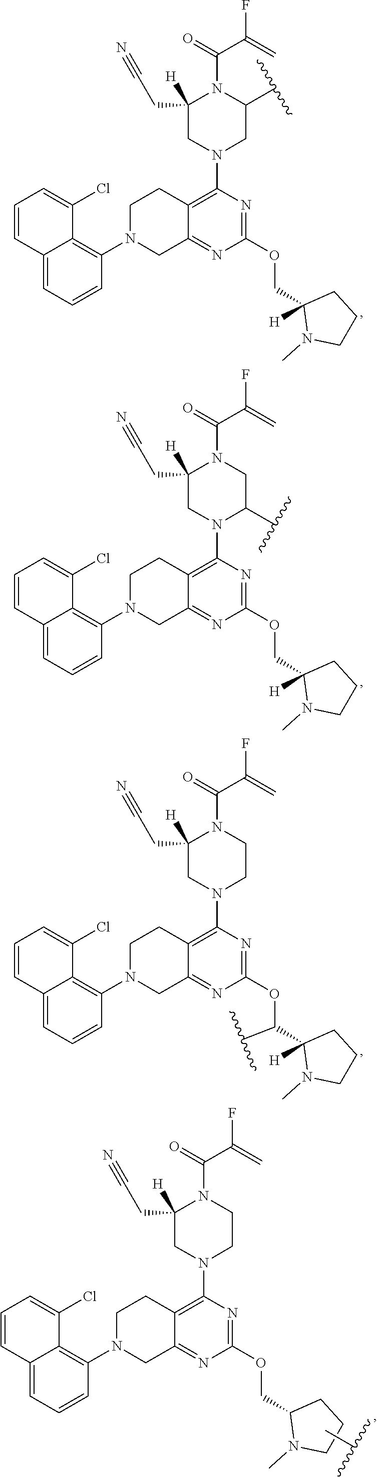

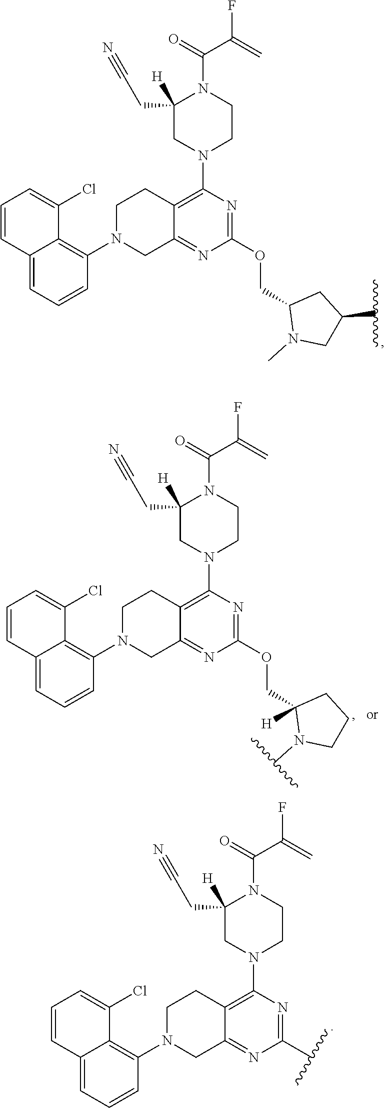

In certain embodiments, the TPL is one of the following:

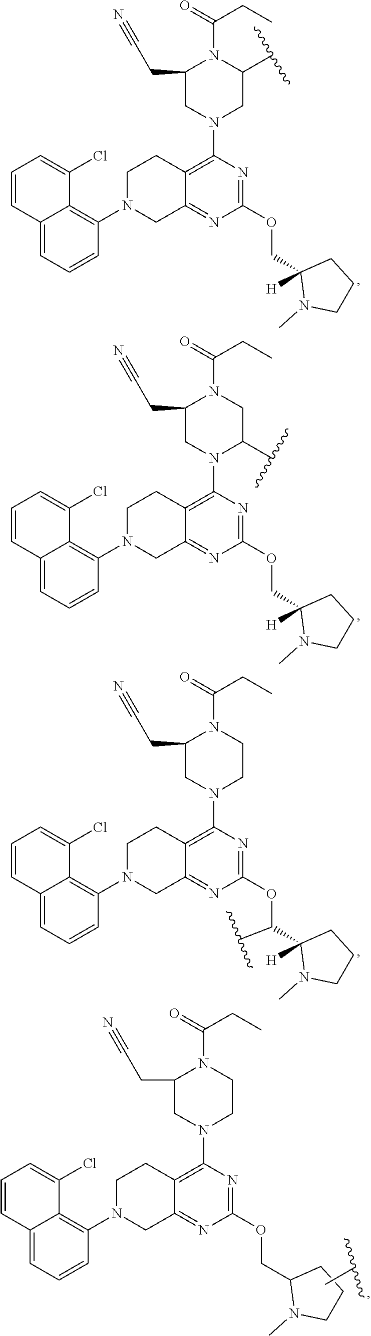

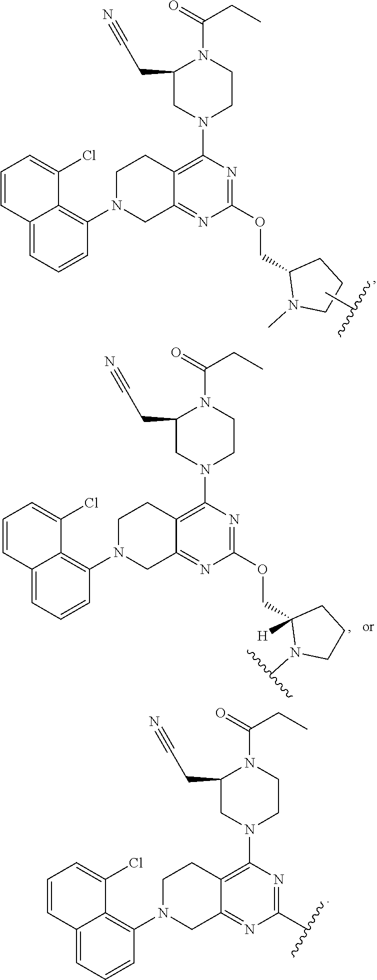

In certain embodiments, the TPL is

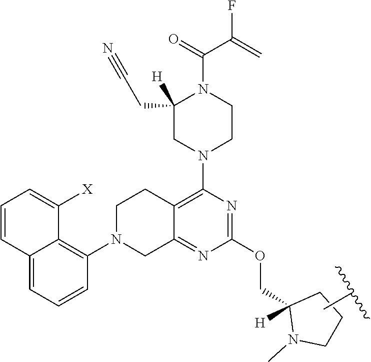

wherein X is hydrogen, halo, C₁₋₆ alkyl, amino or C₁₋₆ alkoxy.

In certain embodiments, the TPL is a radical of

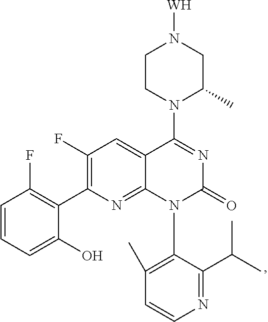

wherein WH is a group that reacts with KRas to form a covalent linkage.

In certain embodiments, the TPL is a radical of

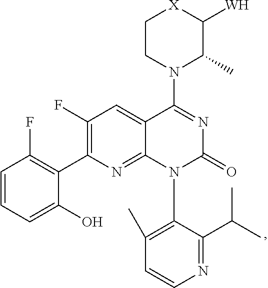

wherein WH is a group that reacts with KRas to form a covalent linkage; X is NH, NR^{a}, CH₂, CHR^{a}, or C(R^{a})₂; and R^{a} is C₁₋₆ alkyl, C₂₋₆ alkenyl, amido, amino, aminoalkyl, or C₁₋₆ alkoxy.

In certain embodiments, the TPL is

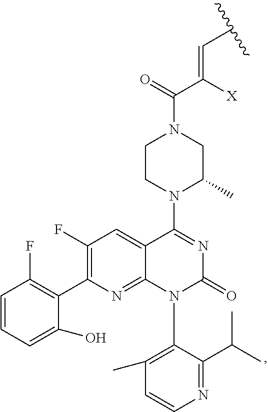

wherein X is hydrogen or halo. In certain embodiments, the TPL is

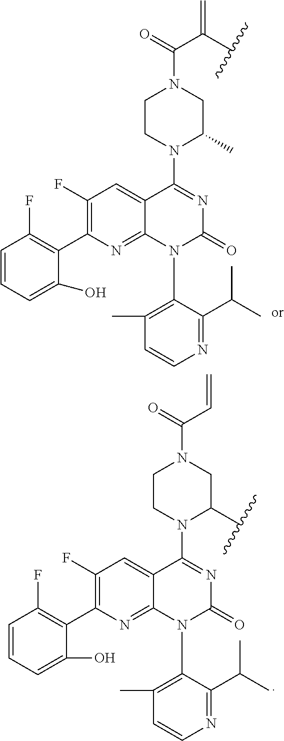

In certain embodiments, the TPL is

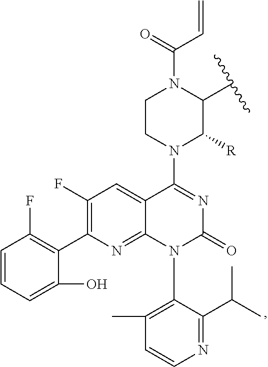

wherein R is H, methyl, ethyl, CH₂OH, CH₂NH₂, CH₂NHR′, OH, or NH₂; and R′ is alkyl, alkenyl, amido, amino, aminoalkyl, or alkoxy. In certain embodiments, the TPL is

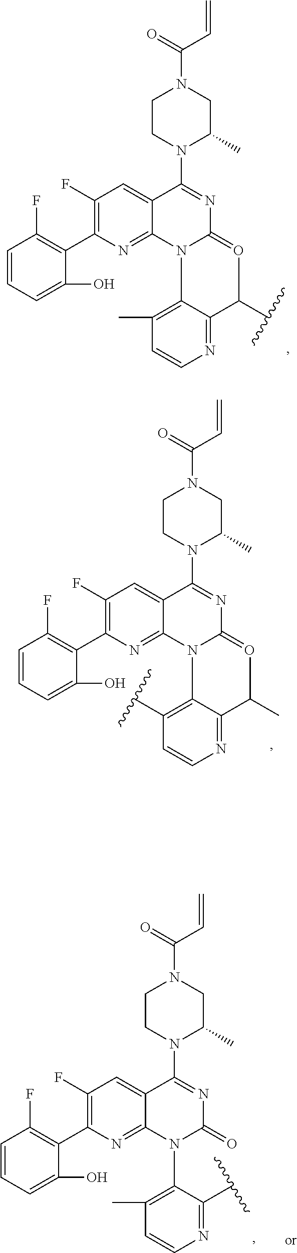

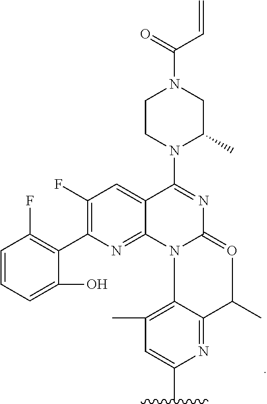

In certain embodiments, the TPL is

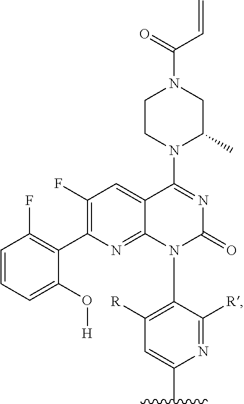

wherein R is H, a linker (e.g., alkyl); and R′ is H or a linker (e.g., alkyl). In certain embodiments, the TPL is

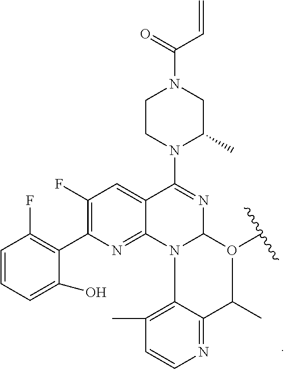

In certain embodiments, the TPL is a radical of

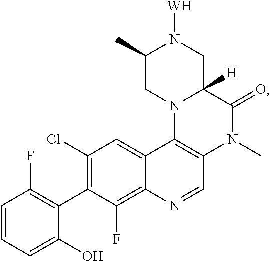

wherein WH is a group that reacts with KRas to form a covalent linkage. In certain embodiments, the TPL is

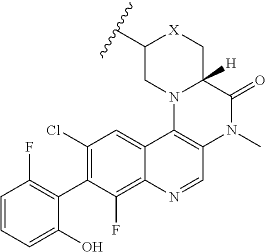

wherein X is NH, NR^{a}, CH₂, CHR^{a}, or C(R^{a})₂; and R^{a} is alkyl, alkenyl, amido, amino, aminoalkyl, or alkoxy.

In certain embodiments, the TPL is

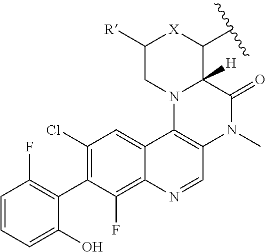

wherein X is NH, NR^{a}, CH₂, CHR^{a}, or C(R^{a})₂; R^{a} is alkyl, alkenyl, amido, amino, aminoalkyl, or alkoxy; and R′ is H, methyl, or ethyl. In certain embodiments, the TPL is

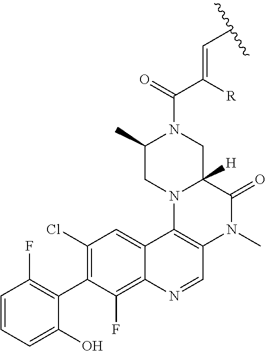

wherein R is hydrogen or halo. In certain embodiments, the TPL is

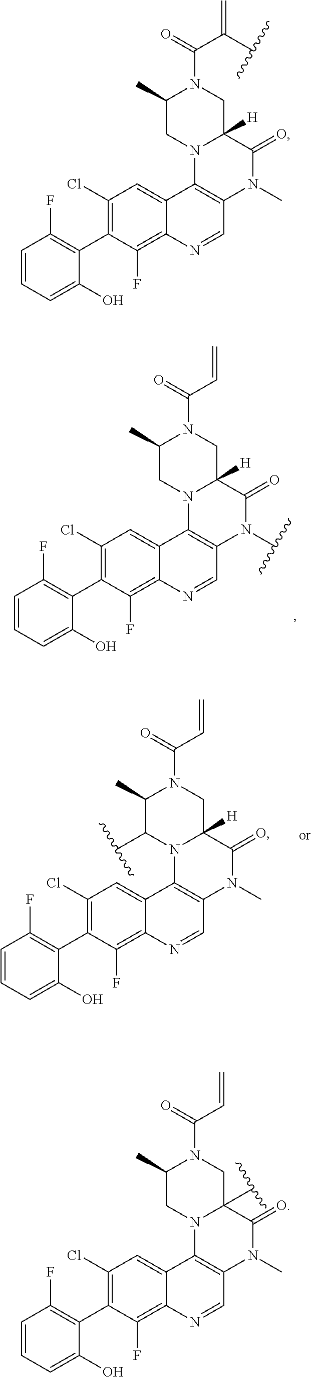 [SMILES: *C(=C)C(=O)N1C[C@@H]2C(=O)N(C)c3cnc4c(F)c(-c5c(O)cccc5F)c(Cl)cc4c3N2C[C@H]1C ; *N1C(=O)[C@H]2CN(C(=O)C=C)[C@H](C)CN2c2c1cnc1c(F)c(-c3c(O)cccc3F)c(Cl)cc21 ; *C1[C@@H](C)N(C(=O)C=C)C[C@@H]2C(=O)N(C)c3cnc4c(F)c(-c5c(O)cccc5F)c(Cl)cc4c3N12 ; *C12CN(C(=O)C=C)[C@H](C)CN1c1c(cnc3c(F)c(-c4c(O)cccc4F)c(Cl)cc13)N(C)C2=O]

In certain embodiments, the TPL is a moiety that binds to a mutated Kirsten rat sarcoma 2 viral oncogene homolog. Compounds that bind Binders of mutated Kirsten rat sarcoma 2 viral oncogene homolog are reported in the literature, which include:

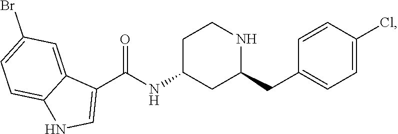 [SMILES: O=C(N[C@@H]1CCN[C@@H](Cc2ccc(Cl)cc2)C1)c1c[nH]c2ccc(Br)cc12]

as described in Jansen, J. M. et al., 24th Int Symp Med Chem (August 28-September 1, Manchester) 2016, Abstract LE007;

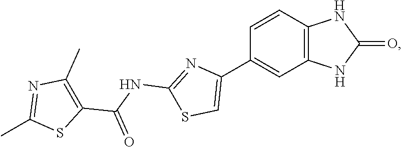 [SMILES: Cc1nc(C)c(C(=O)Nc2nc(-c3ccc4[nH]c(=O)[nH]c4c3)cs2)s1]

as described in Rabizadeh, S. et al., WO 2016/161361;

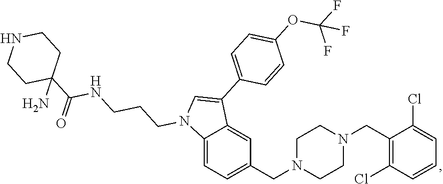 [SMILES: NC1(C(=O)NCCCn2cc(-c3ccc(OC(F)(F)F)cc3)c3cc(CN4CCN(Cc5c(Cl)cccc5Cl)CC4)ccc32)CCNCC1]

as described in Welsch, M. E. et al., Cell 2017, vol. 168(5), page 878;

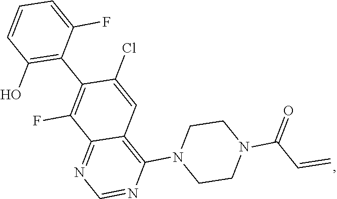 [SMILES: C=CC(=O)N1CCN(c2ncnc3c(F)c(-c4c(O)cccc4F)c(Cl)cc23)CC1]

as described in Wijeratne, A. et al., ACS Med Chem Lett 2018, vol. 9(6), page 557.

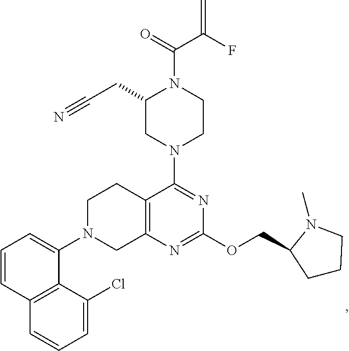 [SMILES: C=C(F)C(=O)N1CCN(c2nc(OC[C@@H]3CCCN3C)nc3c2CCN(c2cccc4cccc(Cl)c24)C3)C[C@@H]1CC#N]

as described in Blake, J. et al., WO 2019/099524

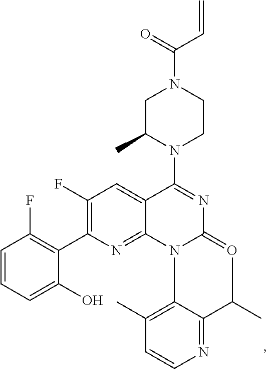

as described in Lanman, B. et al., WO 2018/217651

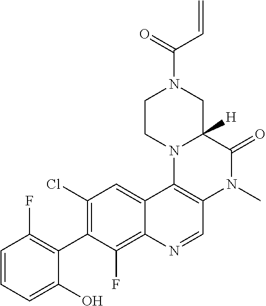 [SMILES: C=CC(=O)N1CCN2c3c(cnc4c(F)c(-c5c(O)cccc5F)c(Cl)cc34)N(C)C(=O)[C@H]2C1]

as described in Kettle, J. et al., WO 2019/110751.

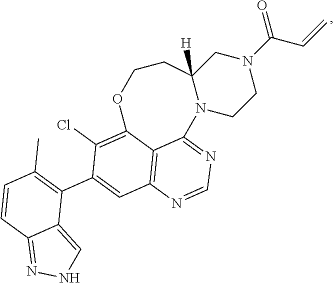 [SMILES: C=CC(=O)N1CCN2c3ncnc4cc(-c5c(C)ccc6n[nH]cc56)c(Cl)c(c34)OCC[C@@H]2C1]

as described in Kettle, J. et al., WO 2018/206539.

In certain embodiments, the TPL is a radical of one of the above compounds, which is attached to L through a modifiable oxygen, nitrogen, or carbon atom.

### Moiety for ER

In certain embodiments, the TPL is a moiety that binds to the estrogen receptor (ER). Exemplary compounds that bind to ER are reported in the literature, such as raloxifene, H3B-6545, and AZD9496. A radical of such compounds reported in the literature that bind ER are amenable for use in the present invention.

In certain embodiments, the TPL is

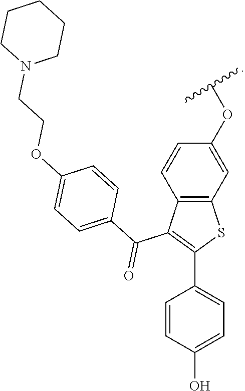 [SMILES: *Oc1ccc2c(C(=O)c3ccc(OCCN4CCCCC4)cc3)c(-c3ccc(O)cc3)sc2c1]

In certain embodiments, the TPL is

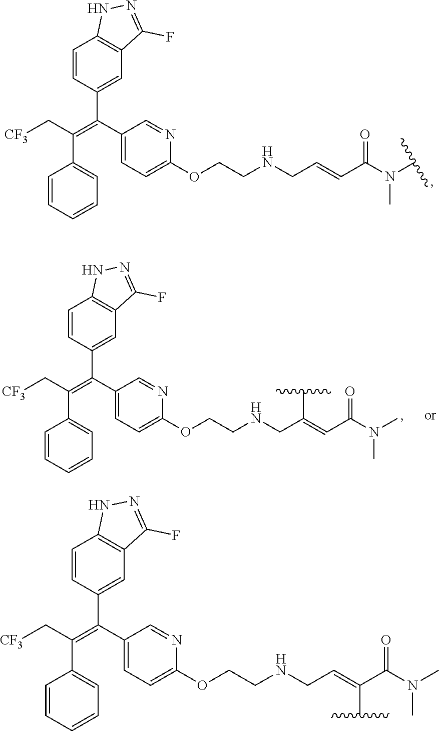 [SMILES: *N(C)C(=O)/C=C/CNCCOc1ccc(/C(=C(/CC(F)(F)F)c2ccccc2)c2ccc3[nH]nc(F)c3c2)cn1 ; */C(=C\C(=O)N(C)C)CNCCOc1ccc(/C(=C(/CC(F)(F)F)c2ccccc2)c2ccc3[nH]nc(F)c3c2)cn1 ; */C(=C\CNCCOc1ccc(/C(=C(/CC(F)(F)F)c2ccccc2)c2ccc3[nH]nc(F)c3c2)cn1)C(=O)N(C)C]

In certain embodiments, the TPL is a radical of

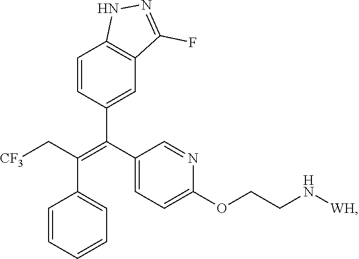

wherein WH is a group that reacts with ER to form a covalent linkage. In certain embodiments, the TPL is a radical of

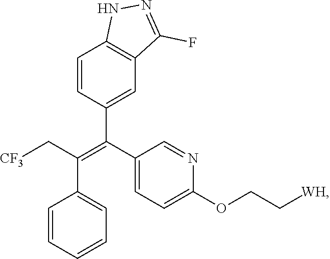

wherein WH is a group that reacts with ER to form a covalent linkage.

In certain embodiments, the TPL is a radical of

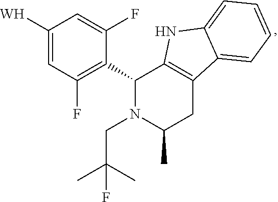

wherein WH is a group that reacts with ER to form a covalent linkage. In certain embodiments, the TPL is

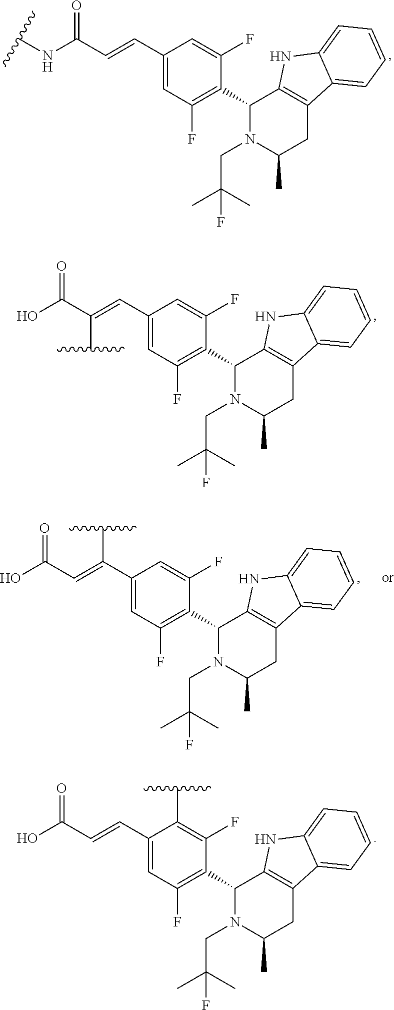

In certain embodiments, the TPL is

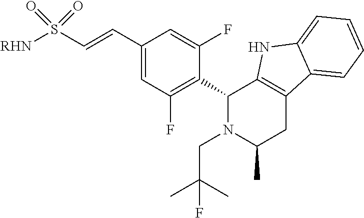

wherein R is H or alkyl.

In certain embodiments, the TPL is a radical of one of the above compounds, which is attached to L through a modifiable oxygen, nitrogen, or carbon atom.

In certain embodiments, the TPL is a moiety that is an activator, inhibitor, and/or binds to the estrogen receptor (ER). Compounds that activate, inhibit, and/or bind to the ER are reported in the literature, which include:

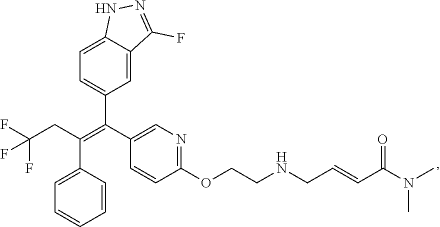 [SMILES: CN(C)C(=O)/C=C/CNCCOc1ccc(/C(=C(/CC(F)(F)F)c2ccccc2)c2ccc3[nH]nc(F)c3c2)cn1]

as described in Bock, M. et al., in US20160347717.

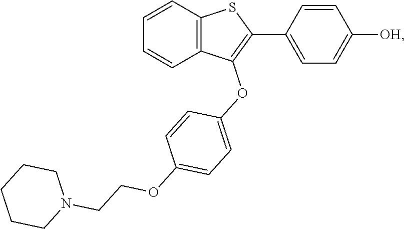 [SMILES: Oc1ccc(-c2sc3ccccc3c2Oc2ccc(OCCN3CCCCC3)cc2)cc1]

as described in Palkowitz, A., in U.S. Pat. No. 5,488,058.

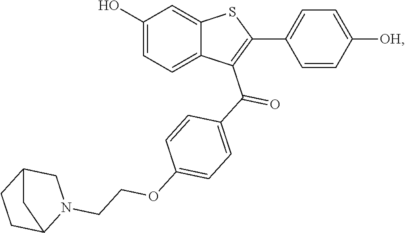 [SMILES: O=C(c1ccc(OCCN2CC3CCC2C3)cc1)c1c(-c2ccc(O)cc2)sc2cc(O)ccc12]

as described in Cameron, K. et al., in WO1995010513.

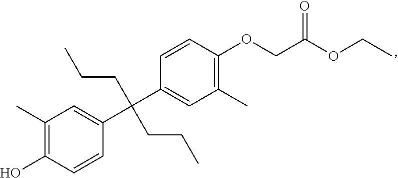 [SMILES: CCCC(CCC)(c1ccc(O)c(C)c1)c1ccc(OCC(=O)OCC)c(C)c1]

as described in Nanjyo, S. et al., in Bioorg Med Chem 2019, 27(10): 1952.

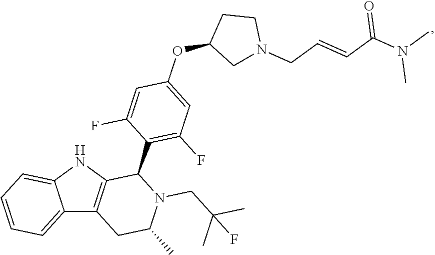 [SMILES: C[C@@H]1Cc2c([nH]c3ccccc23)[C@@H](c2c(F)cc(O[C@H]3CCN(C/C=C/C(=O)N(C)C)C3)cc2F)N1CC(C)(C)F]

as described in Yang, F. et al., in WO2019223715.

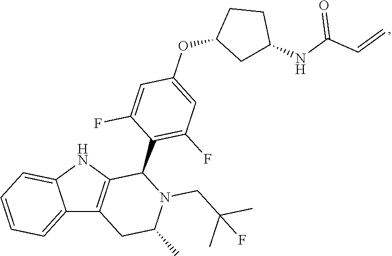 [SMILES: C=CC(=O)N[C@H]1CC[C@@H](Oc2cc(F)c([C@@H]3c4[nH]c5ccccc5c4C[C@@H](C)N3CC(C)(C)F)c(F)c2)C1]

as described in Yang, F. et al., in WO2019223715.

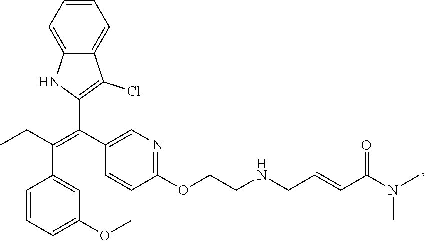 [SMILES: CC/C(=C(/c1ccc(OCCNC/C=C/C(=O)N(C)C)nc1)c1[nH]c2ccccc2c1Cl)c1cccc(OC)c1]

as described in Duan, S. et al., in WO2020125640.

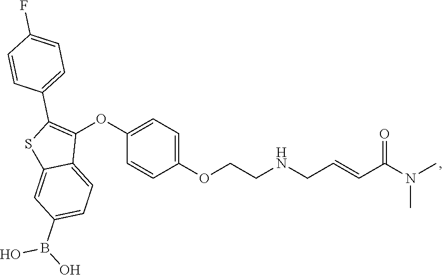 [SMILES: CN(C)C(=O)/C=C/CNCCOc1ccc(Oc2c(-c3ccc(F)cc3)sc3cc(B(O)O)ccc23)cc1]

as described in Wang, G. et al., in WO2020055973.

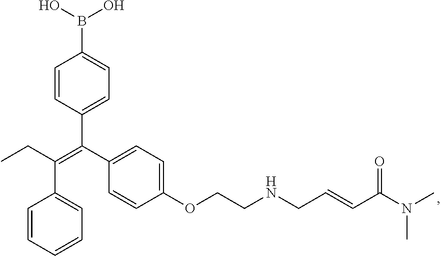 [SMILES: CC/C(=C(/c1ccc(OCCNC/C=C/C(=O)N(C)C)cc1)c1ccc(B(O)O)cc1)c1ccccc1]

as described in Wang, G. et al., in WO2020055973.

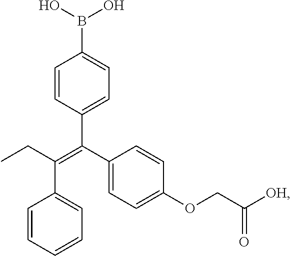 [SMILES: CC/C(=C(/c1ccc(OCC(=O)O)cc1)c1ccc(B(O)O)cc1)c1ccccc1]

as described in Ruenitz, P., in 219th Am Chem Soc (ACS) Natl Meet⋅2000-03-26/2000-03-30⋅San Francisco, United States⋅Abst MEDI 330.

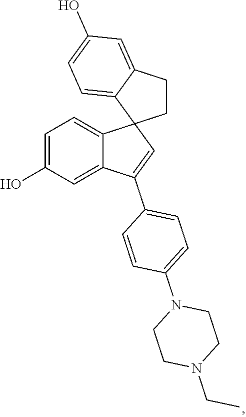 [SMILES: CCN1CCN(c2ccc(C3=CC4(CCc5cc(O)ccc54)c4ccc(O)cc43)cc2)CC1]

as described in Watanabe, N. et al., in Bioorg Med Chem Lett 2003, 13(24): 4317.

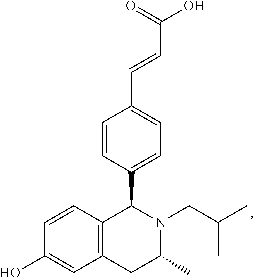 [SMILES: CC(C)CN1[C@H](c2ccc(/C=C/C(=O)O)cc2)c2ccc(O)cc2C[C@H]1C]

as described in Scott, J. et al., in ACS Med Chem Lett 2016, 7(1): 94.

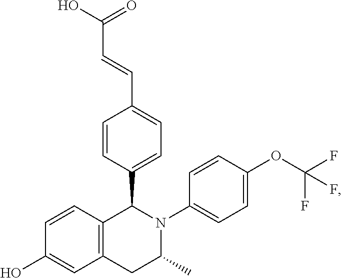 [SMILES: C[C@@H]1Cc2cc(O)ccc2[C@@H](c2ccc(/C=C/C(=O)O)cc2)N1c1ccc(OC(F)(F)F)cc1]

as described in Scott, J. et al., in ACS Med Chem Lett 2016,

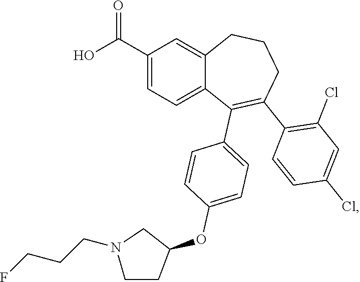 [SMILES: O=C(O)c1ccc2c(c1)CCCC(c1ccc(Cl)cc1Cl)=C2c1ccc(O[C@H]2CCN(CCCF)C2)cc1]

as described in Bouaboula, M. et al., in US2020392081.

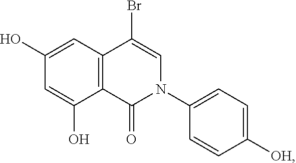 [SMILES: O=c1c2c(O)cc(O)cc2c(Br)cn1-c1ccc(O)cc1]

as described in Dalton, J. et al., in WO2008091555.

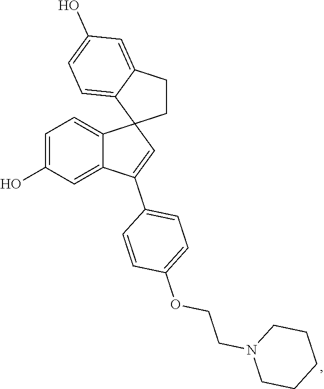 [SMILES: Oc1ccc2c(c1)CCC21C=C(c2ccc(OCCN3CCCCC3)cc2)c2cc(O)ccc21]

as described in Watanabe, N. et al., in J Med Chem 2003, 46(19): 3961.

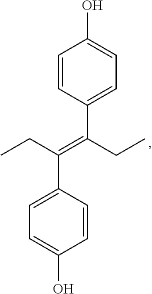 [SMILES: CC/C(=C(/CC)c1ccc(O)cc1)c1ccc(O)cc1]

as described in Nanjyo, S. et al., in Bioorg Med Chem 2019, 27(10): 1952.

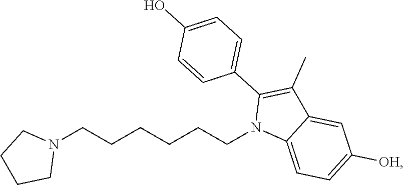 [SMILES: Cc1c(-c2ccc(O)cc2)n(CCCCCCN2CCCC2)c2ccc(O)cc12]

as described in Miller, C. et al., in J Med Chem 2001, 44(11):
1654.

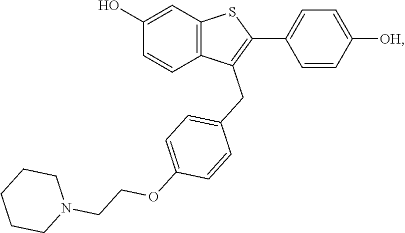 [SMILES: Oc1ccc(-c2sc3cc(O)ccc3c2Cc2ccc(OCCN3CCCCC3)cc2)cc1]

as described in Cameron, K. et al., in WO1995010513.

In certain embodiments, the TPL is a radical of one of the above compounds, which is attached to L through a modifiable oxygen, nitrogen, or carbon atom.

### Moiety for ALK

In certain embodiments, the TPL is a moiety that binds to anaplastic lymphoma kinase (ALK). Exemplary compounds that bind to ALK are reported in the literature, such as ceritinib. A radical of such compounds reported in the literature that bind ALK are amenable for use in the present invention.

In certain embodiments, the TPL is one of the following:

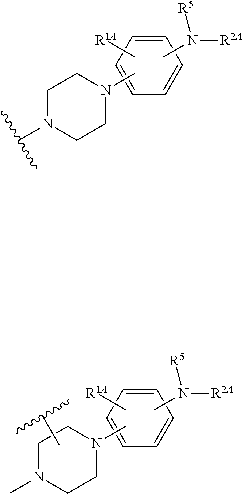

wherein:
R^{1A} is hydrogen, halo, hydroxyl, C₁₋₄ alkyl, C₁₋₄ alkoxyl, or N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl)-N(R^{5A})-(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, C₁₋₄ alkoxyl, and —P(O)(C₁₋₆ alkyl)₂; and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

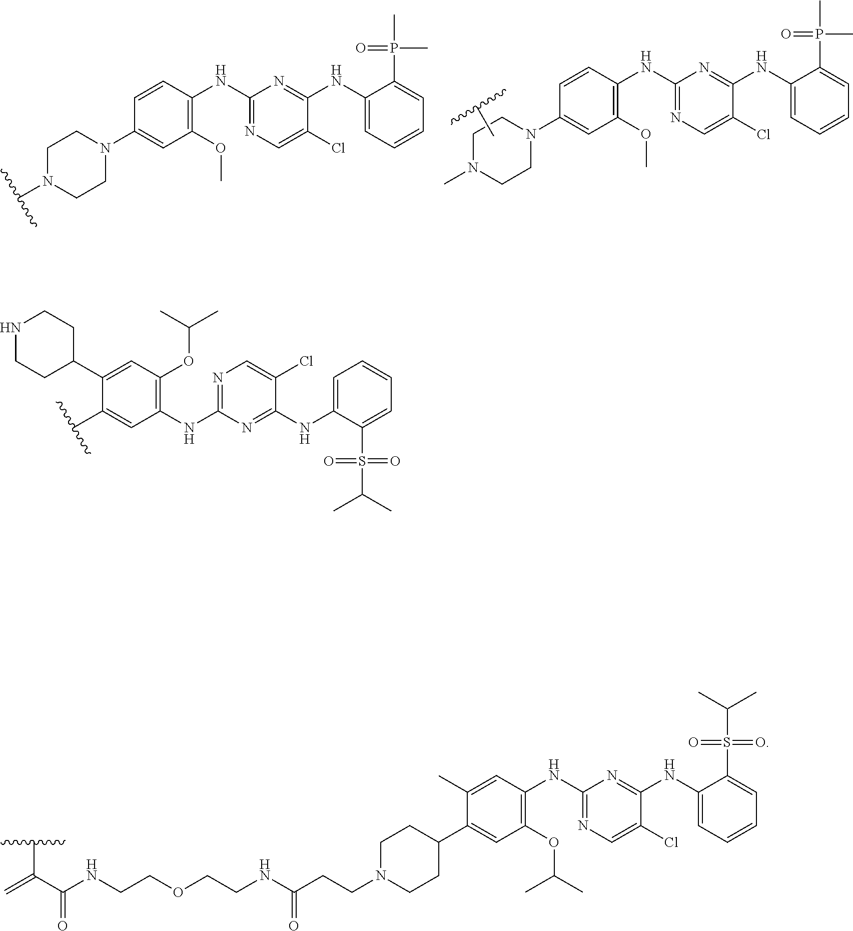

In certain embodiments, the TPL is

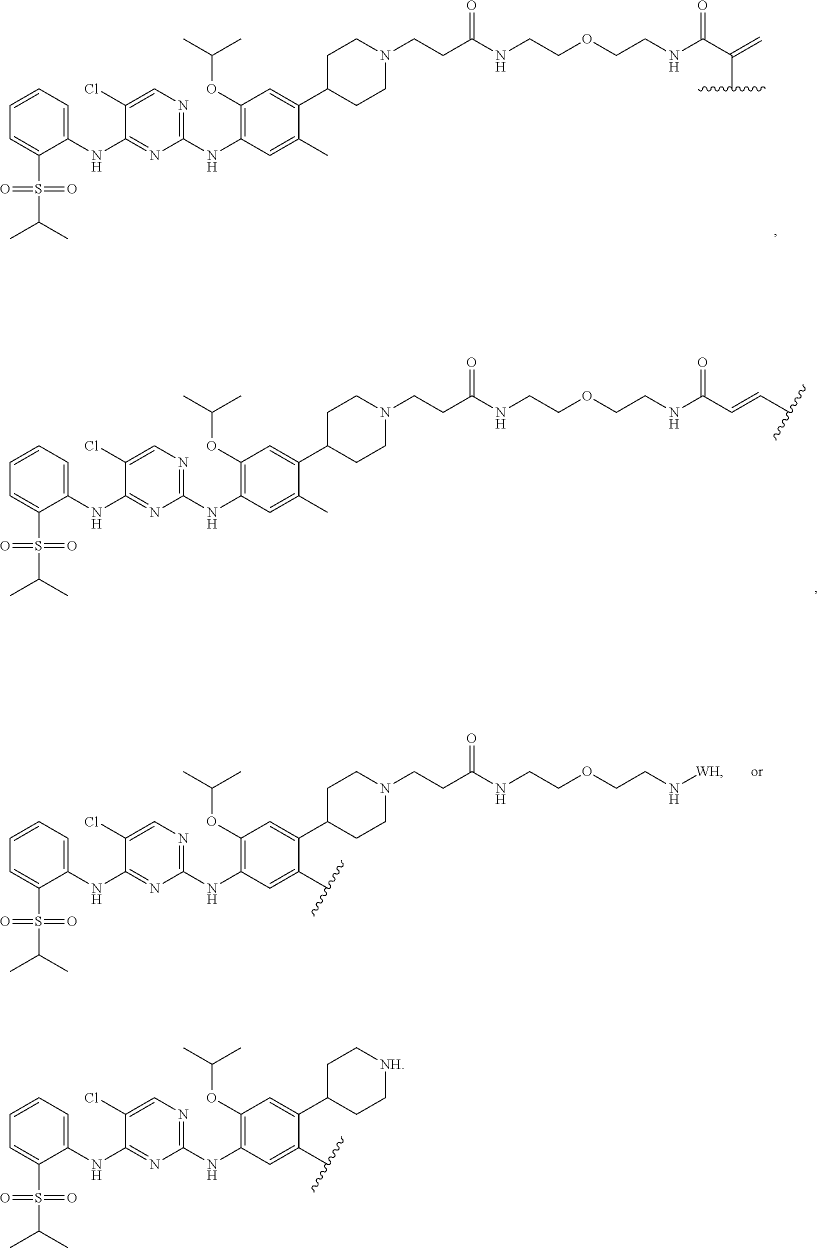

In certain embodiments, the TPL is a radical of one of the above compounds, which is attached to L through a modifiable oxygen, nitrogen, or carbon atom.

In certain embodiments, the TPL is a moiety that binds to and/or inhibits ALK. Compounds that bind and/or inhibit ALK are reported in the literature, which include EML4. Additional exemplary compounds that bind to and/or inhibit ALK and/or an ALK-fusion protein include:

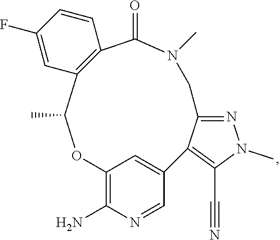 [SMILES: C[C@H]1Oc2cc(cnc2N)-c2c(nn(C)c2C#N)CN(C)C(=O)c2ccc(F)cc21]

as described in Wang, Y., et al., WO2017148325;

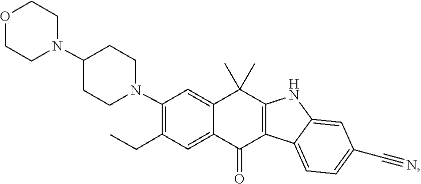 [SMILES: CCc1cc2c(cc1N1CCC(N3CCOCC3)CC1)C(C)(C)c1[nH]c3cc(C#N)ccc3c1C2=O]

as described in Kodama, T., et al., Mol Cancer Ther 2014, 13(12): 2910;

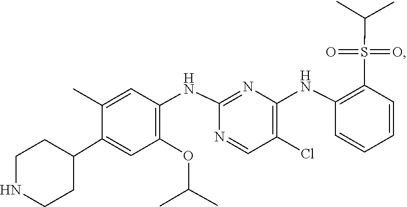 [SMILES: Cc1cc(Nc2ncc(Cl)c(Nc3ccccc3S(=O)(=O)C(C)C)n2)c(OC(C)C)cc1C1CCNCC1]

as described in Marsilje, T. H., et al., J Med Chem. 2013 Jul. 25; 56(14):5675-90 and Chen, J., et al., J Med Chem. 2013 Jul. 25; 56(14):5673-4;

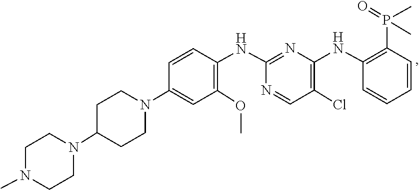

as described in Zhang, S., et al., Clin Cancer Res. 2016 Nov. 15; 22(22):5527-5538;

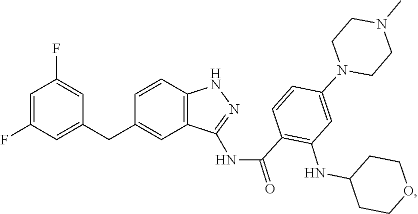 [SMILES: CN1CCN(c2ccc(C(=O)Nc3n[nH]c4ccc(Cc5cc(F)cc(F)c5)cc34)c(NC3CCOCC3)c2)CC1]

as described in Ardini, E., et al., Mol Cancer Ther. 2016 April; 15(4):628-39;

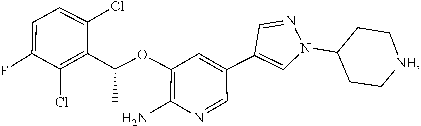 [SMILES: C[C@@H](Oc1cc(-c2cnn(C3CCNCC3)c2)cnc1N)c1c(Cl)ccc(F)c1Cl]

as described in Cui, J. J., et al., J Med Chem. 2011 Sep. 22; 54(18):6342-63;

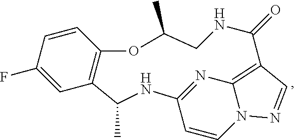 [SMILES: C[C@H]1CNC(=O)c2cnn3ccc(nc23)N[C@H](C)c2cc(F)ccc2O1]

as described in Zhai, D., et al., 108th Annu Meet Am Assoc Cancer Res (AACR)⋅2017-04-01/2017-04-05⋅Washington, D.C., United States⋅Abst 3161, Cancer Res 2017, 77(13);

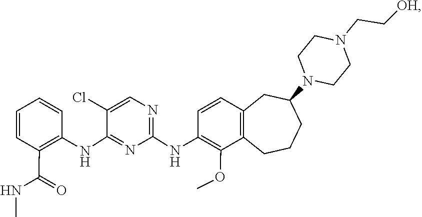 [SMILES: CNC(=O)c1ccccc1Nc1nc(Nc2ccc3c(c2OC)CCC[C@H](N2CCN(CCO)CC2)C3)ncc1Cl]

as described in Jacobs, Martin, J., et al., WO2013134353;

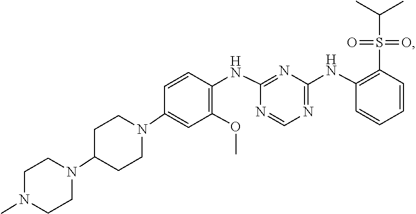 [SMILES: COc1cc(N2CCC(N3CCN(C)CC3)CC2)ccc1Nc1ncnc(Nc2ccccc2S(=O)(=O)C(C)C)n1]

as described in Mori, M., et al., Mol Cancer Ther 2014, 13(2): 329;

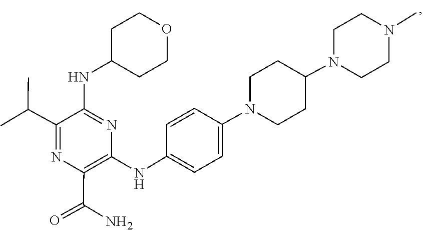 [SMILES: CC(C)c1nc(C(N)=O)c(Nc2ccc(N3CCC(N4CCN(C)CC4)CC3)cc2)nc1NC1CCOCC1]

as described in Shimada, I., et al., WO2010128659;

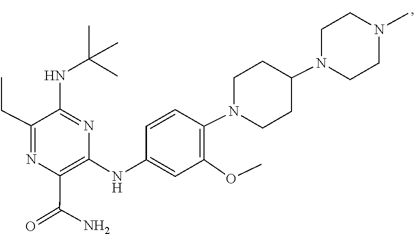 [SMILES: CCc1nc(C(N)=O)c(Nc2ccc(N3CCC(N4CCN(C)CC4)CC3)c(OC)c2)nc1NC(C)(C)C]

as described in Shimada, I., et al., WO2012053606;

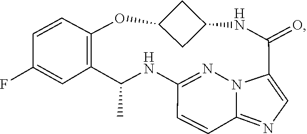 [SMILES: C[C@H]1Nc2ccc3ncc(n3n2)C(=O)N[C@H]2C[C@H](C2)Oc2ccc(F)cc21]

as described in Zhang, Y., et al., WO2019210835;

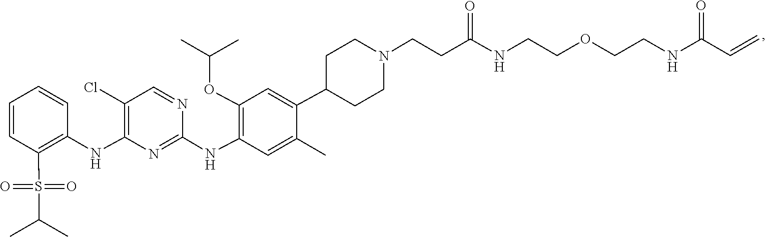 [SMILES: C=CC(=O)NCCOCCNC(=O)CCN1CCC(c2cc(OC(C)C)c(Nc3ncc(Cl)c(Nc4ccccc4S(=O)(=O)C(C)C)n3)cc2C)CC1]

as described in Yan, G., et al., J. Med. Chem 2021, 64(3): 1558.

In certain embodiments, the TPL is a radical of one of the above compounds, which is attached to L through a modifiable oxygen, nitrogen, or carbon atom.

In certain embodiments, the TPL has the formula:

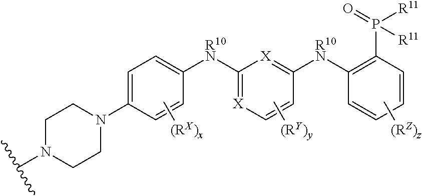

wherein:
each X is independently N or CH;
each instance of R^{X}, R^{Y}, and R^{Z} is independently halogen, —CN, —NO₂, —OR, SR, —NR₂, an optionally substituted C₁₋₆ aliphatic group, an optionally substituted C₁₋₆ aliphatic-Cy group, or Cy;
each R¹⁰ is independently hydrogen or an optionally substituted C₁₋₆ aliphatic group;
each R¹¹ is independently an optionally substituted C₁₋₆ aliphatic group;
Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); and/or
two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur); and
x, y, and z are each independently selected from 0, 1, 2, and 3.

### Moiety for FGFR1

In certain embodiments, the TPL is a moiety that binds to and/or inhibits Fibroblast Growth Factor Receptor 1 (FGFR1). Compounds that bind and/or inhibit FGFR1 are reported in the literature, which include:

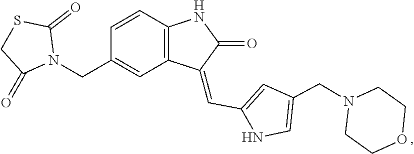 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

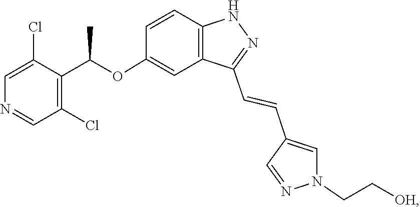 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

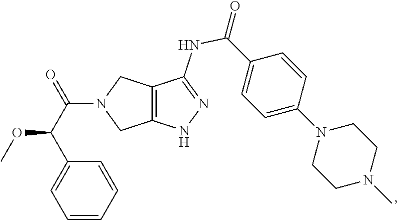

as described in Fancelli, D. et al., J Med Chem 2006, vol. 49(24), page 7247;

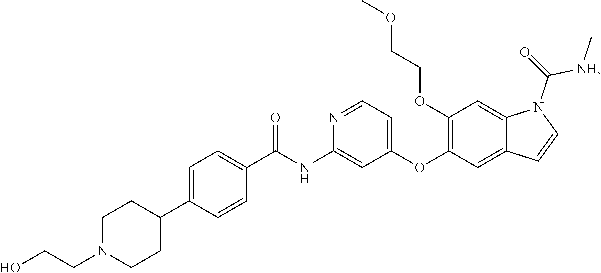 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

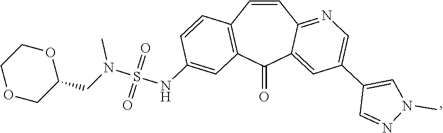 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

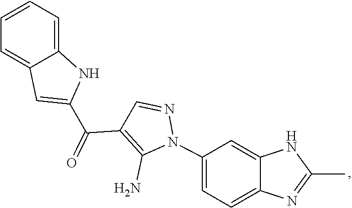 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

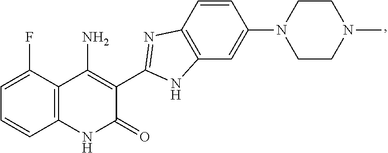 [SMILES: CN1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

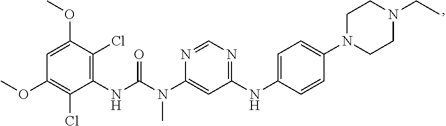 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

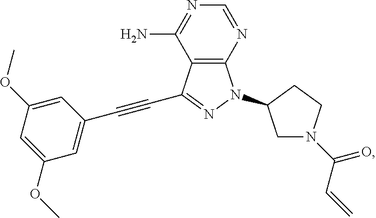 [SMILES: C=CC(=O)N1CC[C@H](n2nc(C#Cc3cc(OC)cc(OC)c3)c3c(N)ncnc32)C1]

as described in Sagara, T. et al., WO 2013/108809;

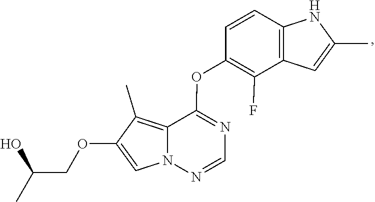 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

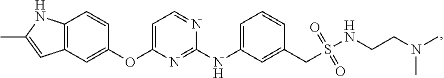

as described in Su, W.-G. et al., WO 2011/060746;

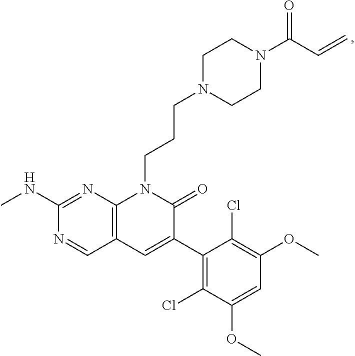 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

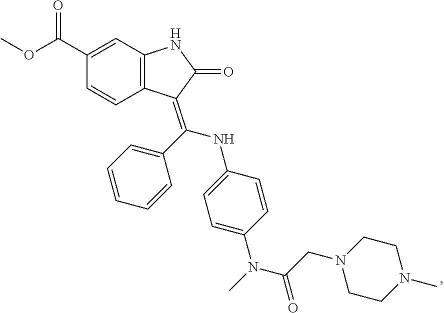

as described in Walters, I. et al., WO 2017/109513;

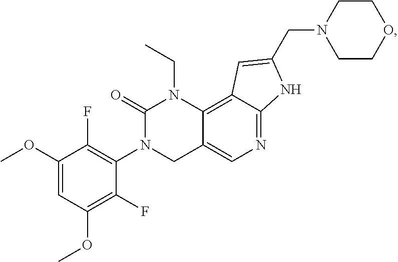 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

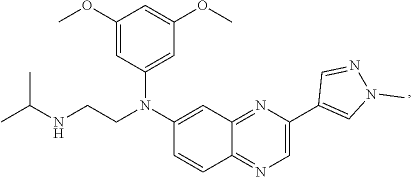 [SMILES: COc1cc(OC)cc(N(CCNC(C)C)c2ccc3ncc(-c4cnn(C)c4)nc3c2)c1]

as described in Xu, X. et al., WO 2018/153373;

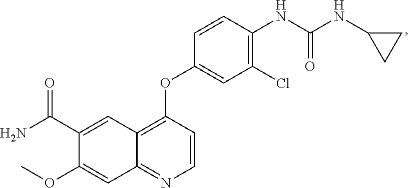 [SMILES: COc1cc2nccc(Oc3ccc(NC(=O)NC4CC4)c(Cl)c3)c2cc1C(N)=O]

as described in Zhang, Y. et al., WO 2019/062637.

In certain embodiments, the TPL is a radical of one of the above compounds, which is attached to L through a modifiable oxygen, nitrogen, or carbon atom.

### Moiety for FGFR2

In certain embodiments, the TPL is a moiety that binds to and/or inhibits Fibroblast Growth Factor Receptor 2 (FGFR2). Compounds that bind and/or inhibit FGFR2 are reported in the literature, which include:

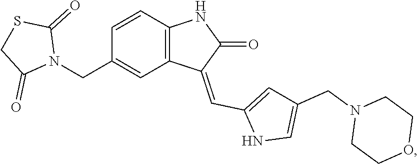 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

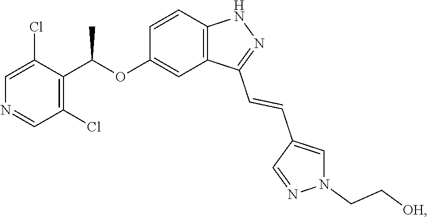 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

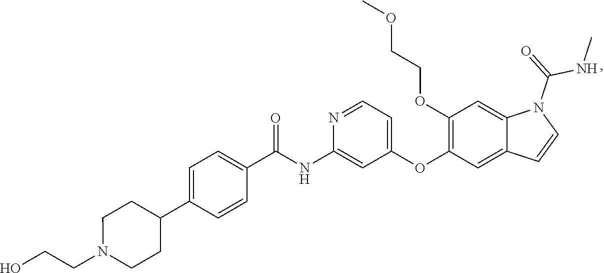 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

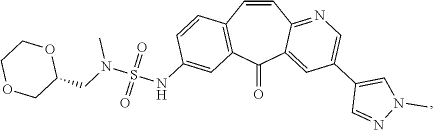 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

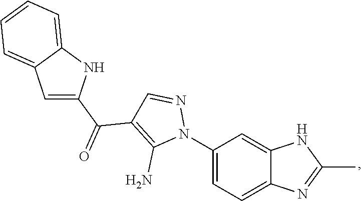 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

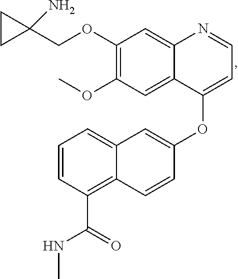 [SMILES: CNC(=O)c1cccc2cc(Oc3ccnc4cc(OCC5(N)CC5)c(OC)cc34)ccc12]

as described in Nguyen, M., et al., 106th Annu Meet Am Assoc Cancer Res (AACR) (April 18-22, Philadelphia) 2015, Abstract 784;

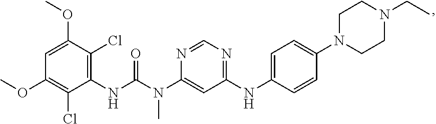 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

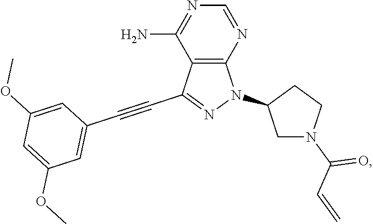 [SMILES: C=CC(=O)N1CC[C@H](n2nc(C#Cc3cc(OC)cc(OC)c3)c3c(N)ncnc32)C1]

as described in Sagara, T. et al., WO 2013/108809;

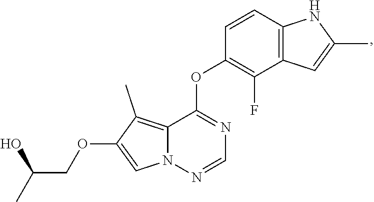 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

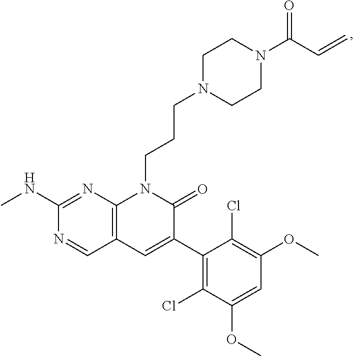 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

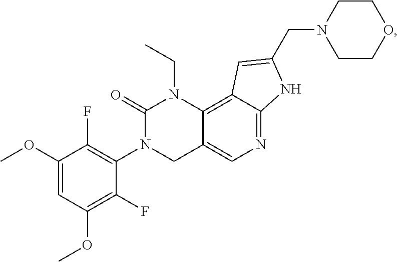 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

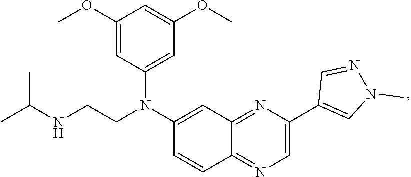 [SMILES: COc1cc(OC)cc(N(CCNC(C)C)c2ccc3ncc(-c4cnn(C)c4)nc3c2)c1]

as described in Xu, X. et al., WO 2018/153373.

### Moiety for FGFR3

In certain embodiments, the TPL is a moiety that binds to and/or inhibits Fibroblast Growth Factor Receptor 3 (FGFR3). Compounds that bind and/or inhibit FGFR3 are reported in the literature, which include:

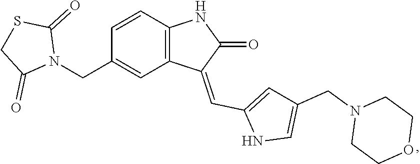 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

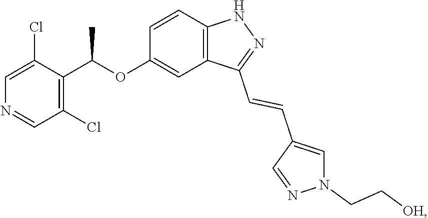 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

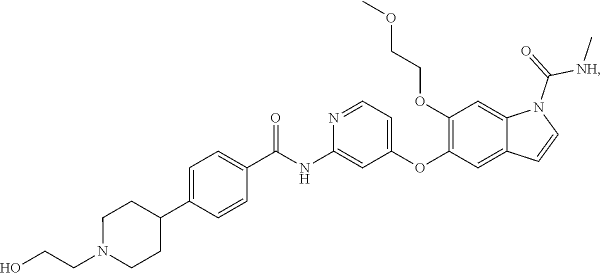 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

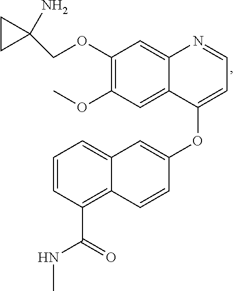 [SMILES: CNC(=O)c1cccc2cc(Oc3ccnc4cc(OCC5(N)CC5)c(OC)cc34)ccc12]

as described in Holmstroem, T. H., et al., Mol Cancer Ther 2019, vol. 18(1), page 28;

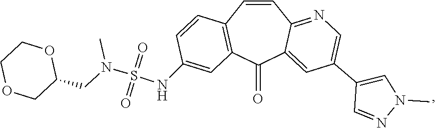 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

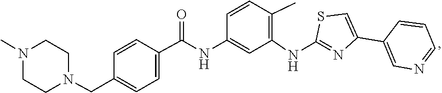 [SMILES: Cc1ccc(NC(=O)c2ccc(CN3CCN(C)CC3)cc2)cc1Nc1nc(-c2cccnc2)cs1]

as described in Moussy, A. et al., WO 2015/082496;

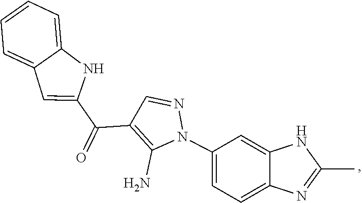 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

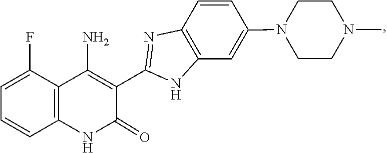 [SMILES: CN1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

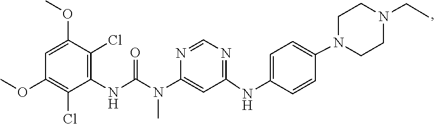 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

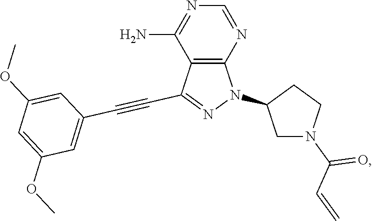 [SMILES: C=CC(=O)N1CC[C@H](n2nc(C#Cc3cc(OC)cc(OC)c3)c3c(N)ncnc32)C1]

as described in Sagara, T. et al., WO 2013/108809;

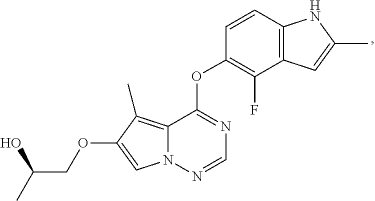 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

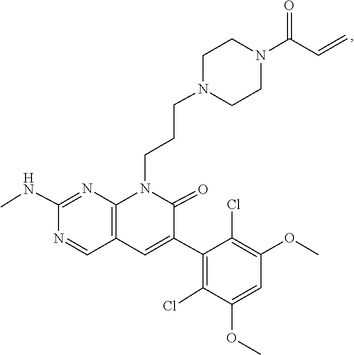 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

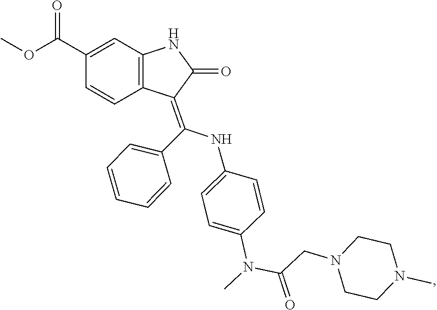

as described in Walters, I. et al., WO 2017/109513;

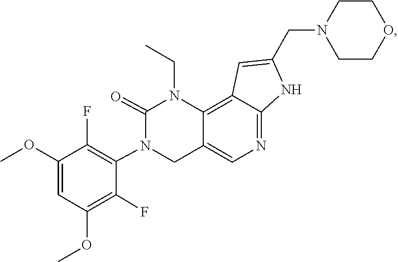 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951.

In certain embodiments, the TPL is a radical of one of the above compounds, which is attached to L through a modifiable oxygen, nitrogen, or carbon atom.

### Moiety for FGFR4

In certain embodiments, the TPL is a moiety that binds to and/or inhibits Fibroblast Growth Factor Receptor 4 (FGFR4). Compounds that bind and/or inhibit FGFR4 are reported in the literature, which include:

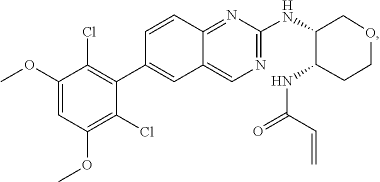 [SMILES: C=CC(=O)N[C@H]1CCOC[C@H]1Nc1ncc2cc(-c3c(Cl)c(OC)cc(OC)c3Cl)ccc2n1]

as described in Bifulco, N. Jr. et al., US2017/174652;

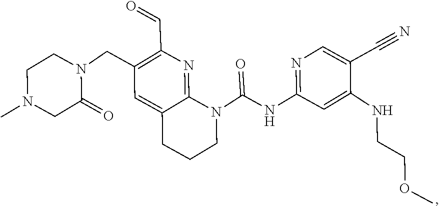

as described in Buschmann, N. et al., WO 2015/059668;

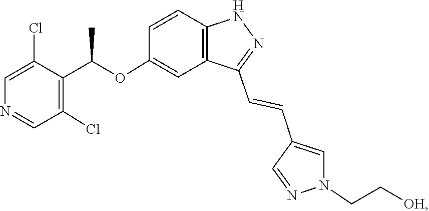 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

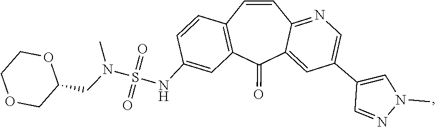 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

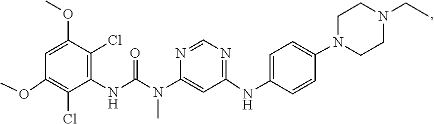 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

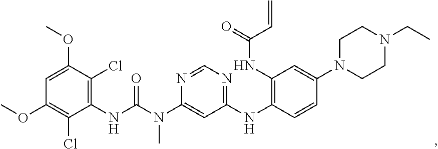 [SMILES: C=CC(=O)Nc1cc(N2CCN(CC)CC2)ccc1Nc1cc(N(C)C(=O)Nc2c(Cl)c(OC)cc(OC)c2Cl)ncn1]

as described in Reynolds, D. et al., WO 2015/057938;

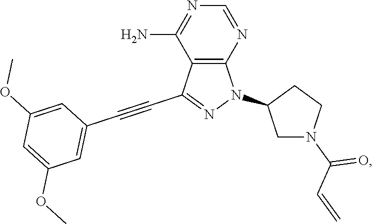 [SMILES: C=CC(=O)N1CC[C@H](n2nc(C#Cc3cc(OC)cc(OC)c3)c3c(N)ncnc32)C1]

as described in Sagara, T. et al., WO 2013/108809;

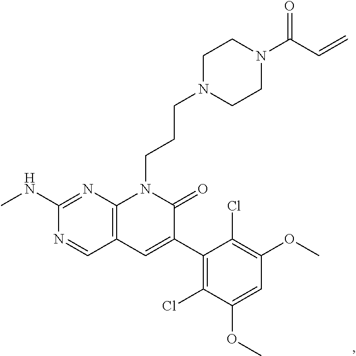 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

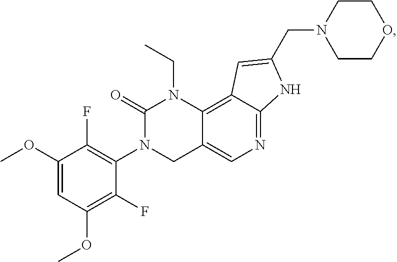 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

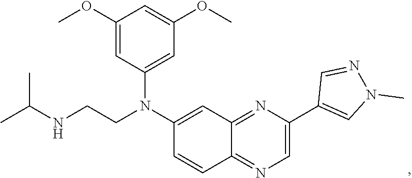 [SMILES: COc1cc(OC)cc(N(CCNC(C)C)c2ccc3ncc(-c4cnn(C)c4)nc3c2)c1]

as described in Xu, X. et al., WO 2018/153373.

In certain embodiments, the TPL is a radical of one of the above compounds, which is attached to L through a modifiable oxygen, nitrogen, or carbon atom.

### Moiety for ERK-1

In certain embodiments, the TPL is a moiety that inhibit extracellular signal-regulated kinase 1 (ERK-1). Compounds that inhibit ERK-1 are reported in the literature, which include:

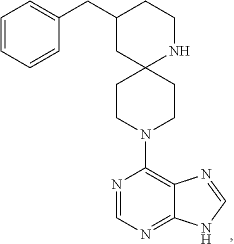 [SMILES: c1ccc(CC2CCNC3(CCN(c4ncnc5[nH]cnc45)CC3)C2)cc1]

as described in Allen, C. E. et al., in Bioorg Med Chem 2013, vol 21(18), page 5707;

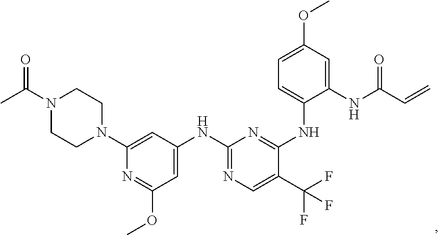 [SMILES: C=CC(=O)Nc1cc(OC)ccc1Nc1nc(Nc2cc(OC)nc(N3CCN(C(C)=O)CC3)c2)ncc1C(F)(F)F]

as described in Haq, N. et al., in WO 2014/124230;

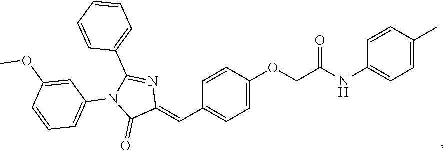

as described in Awadallah, F. M. et al., in Eur J Med Chem 2015, vol 94, page 397;

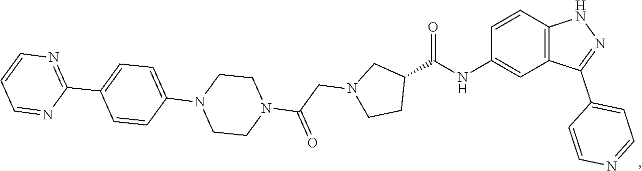 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

as described in Huang, P. Q. et al., in WO 2016/161160;

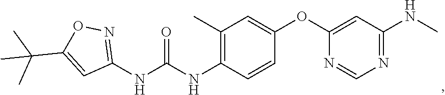 [SMILES: CNc1cc(Oc2ccc(NC(=O)Nc3cc(C(C)(C)C)on3)c(C)c2)ncn1]

as described in Chen, Y. et al., in Eur J Med Chem 2017, vol 127, page 997;

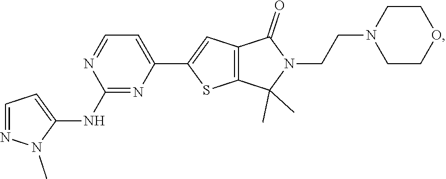 [SMILES: Cn1nccc1Nc1nccc(-c2cc3c(s2)C(C)(C)N(CCN2CCOCC2)C3=O)n1]

as described in Cortez, G. S. et al., in WO 2016/106029;

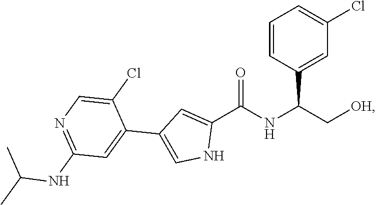 [SMILES: CC(C)Nc1cc(-c2c[nH]c(C(=O)N[C@H](CO)c3cccc(Cl)c3)c2)c(Cl)cn1]

as described in Huang, P. Q. et al., in WO 2016/161160;

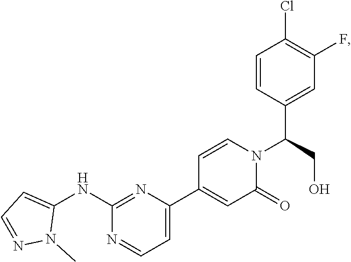 [SMILES: Cn1nccc1Nc1nccc(-c2ccn([C@H](CO)c3ccc(Cl)c(F)c3)c(=O)c2)n1]

as described in Huang, P. Q. et al., in WO 2016/161160;

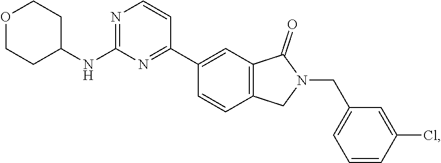 [SMILES: O=C1c2cc(-c3ccnc(NC4CCOCC4)n3)ccc2CN1Cc1cccc(Cl)c1]

as described in Ji, D. Z. et al., in Eur J Med Chem 2019, vol 164, page 334;

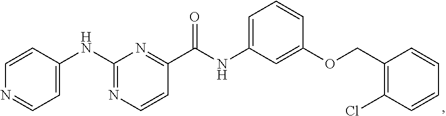 [SMILES: O=C(Nc1cccc(OCc2ccccc2Cl)c1)c1ccnc(Nc2ccncc2)n1]

as described in Kim, E. E. K. et al., in KR2012/092768;

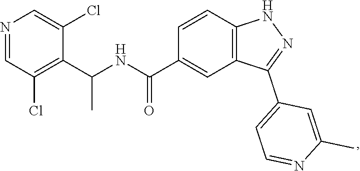 [SMILES: Cc1cc(-c2n[nH]c3ccc(C(=O)NC(C)c4c(Cl)cncc4Cl)cc23)ccn1]

as described in Li, L. et al., in Bioorg Med Chem Lett 2016, vol 26(11), page 2600;

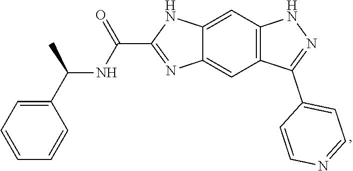 [SMILES: C[C@@H](NC(=O)c1nc2cc3c(-c4ccncc4)n[nH]c3cc2[nH]1)c1ccccc1]

as described in Liu, S. et al., in WO 2019/076336;

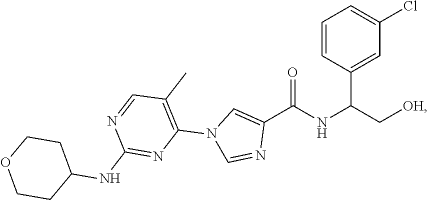 [SMILES: Cc1cnc(NC2CCOCC2)nc1-n1cnc(C(=O)NC(CO)c2cccc(Cl)c2)c1]

as described in Venkatesan, A. M. et al., in U.S. Pat. No. 9,896,445;

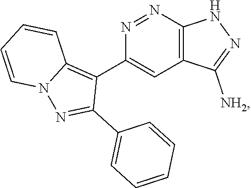 [SMILES: Nc1n[nH]c2nnc(-c3c(-c4ccccc4)nn4ccccc34)cc12]

as described in Zhang, C. et al., in J Pharmacol Exp Ther 2019, vol 370(2), page 206.

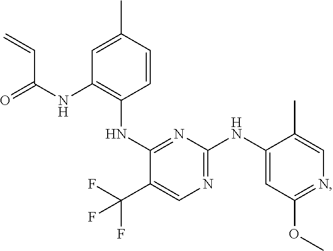 [SMILES: C=CC(=O)Nc1cc(C)ccc1Nc1nc(Nc2cc(OC)ncc2C)ncc1C(F)(F)F]

as described in Haq, N. et al., in WO 2014/124230.

In certain embodiments, the TPL is a radical of one of the above compounds, which is attached to L through a modifiable oxygen, nitrogen, or carbon atom.

### Moiety for ERK-2

In certain embodiments, the TPL is a moiety that inhibits extracellular signal-regulated kinase 2 (ERK-2). Compounds that inhibit ERK-2 are reported in the literature, which include:

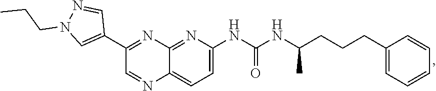 [SMILES: CCCn1cc(-c2cnc3ccc(NC(=O)N[C@H](C)CCCc4ccccc4)nc3n2)cn1]

as described in Gerlach, M. et al., in WO 2012/136691;

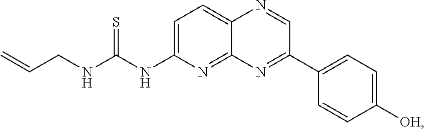 [SMILES: C=CCNC(=S)Nc1ccc2ncc(-c3ccc(O)cc3)nc2n1]

as described in Guenther, E. et al., in WO 2004/104002;

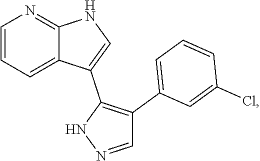 [SMILES: Clc1cccc(-c2cn[nH]c2-c2c[nH]c3ncccc23)c1]

as described in Fairfax, D. et al., in WO 2012/094313;

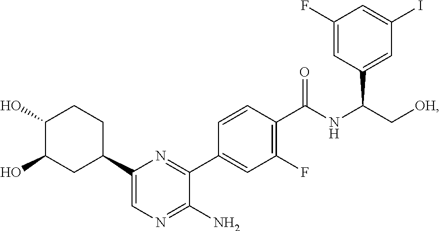 [SMILES: Nc1ncc([C@@H]2CC[C@@H](O)[C@H](O)C2)nc1-c1ccc(C(=O)N[C@H](CO)c2cc(F)cc(I)c2)c(F)c1]

as described in Bagdanoff, J. T. et al., in WO 2015/066188;

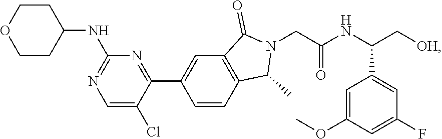 [SMILES: COc1cc(F)cc([C@@H](CO)NC(=O)CN2C(=O)c3cc(-c4nc(NC5CCOCC5)ncc4Cl)ccc3[C@H]2C)c1]

as described in Berdini, V. et al., in WO 2017/068412;

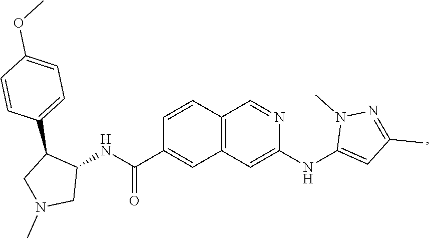 [SMILES: COc1ccc([C@@H]2CN(C)C[C@H]2NC(=O)c2ccc3cnc(Nc4cc(C)nn4C)cc3c2)cc1]

as described in Blake, J. et al., in WO 2014/036015;

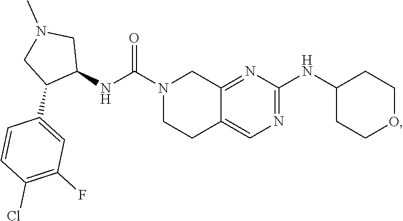 [SMILES: CN1C[C@@H](NC(=O)N2CCc3cnc(NC4CCOCC4)nc3C2)[C@H](c2ccc(Cl)c(F)c2)C1]

as described in Blake, J. F. et al., in WO 2012/118850;

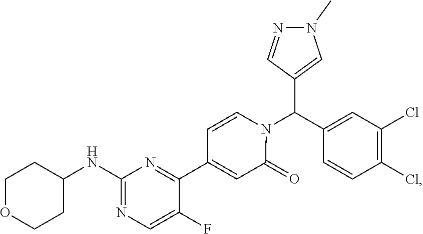 [SMILES: Cn1cc(C(c2ccc(Cl)c(Cl)c2)n2ccc(-c3nc(NC4CCOCC4)ncc3F)cc2=O)cn1]

as described in Blake, J. F. et al., in WO 2013/130976;

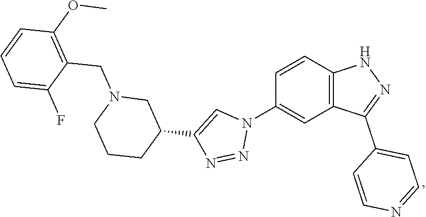 [SMILES: COc1cccc(F)c1CN1CCC[C@@H](c2cn(-c3ccc4[nH]nc(-c5ccncc5)c4c3)nn2)C1]

as described in Boga, S. B. et al., in WO 2012/058127;

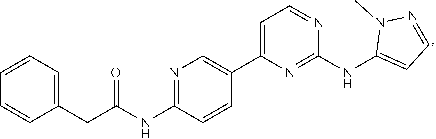 [SMILES: Cn1nccc1Nc1nccc(-c2ccc(NC(=O)Cc3ccccc3)nc2)n1]

as described in Cao, J. et al., in WO 2017/114510;

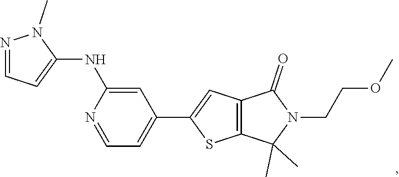 [SMILES: COCCN1C(=O)c2cc(-c3ccnc(Nc4ccnn4C)c3)sc2C1(C)C]

as described in Cortez, G. S. et al., in WO 2016/106009;

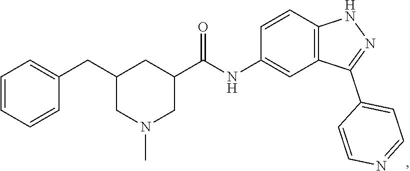 [SMILES: CN1CC(Cc2ccccc2)CC(C(=O)Nc2ccc3[nH]nc(-c4ccncc4)c3c2)C1]

as described in Deng, Y. et al., in WO 2012/030685;

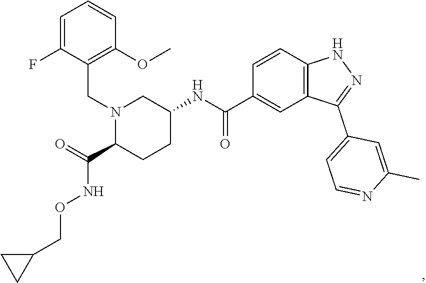 [SMILES: COc1cccc(F)c1CN1C[C@H](NC(=O)c2ccc3[nH]nc(-c4ccnc(C)c4)c3c2)CC[C@H]1C(=O)NOCC1CC1]

as described in Deng, Y. et al., in WO 2011/163330;

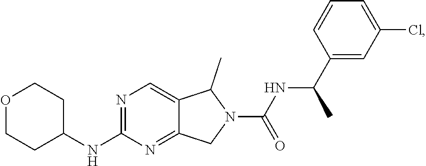 [SMILES: CC1c2cnc(NC3CCOCC3)nc2CN1C(=O)N[C@H](C)c1cccc(Cl)c1]

as described in Dillon, M. P. et al., in WO 2014/047020;

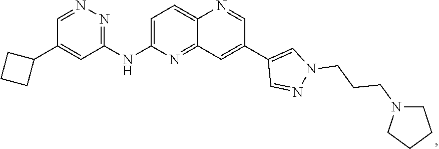 [SMILES: c1nc2ccc(Nc3cc(C4CCC4)cnn3)nc2cc1-c1cnn(CCCN2CCCC2)c1]

as described in Furuyama, H. et al., in WO 2014/109414;

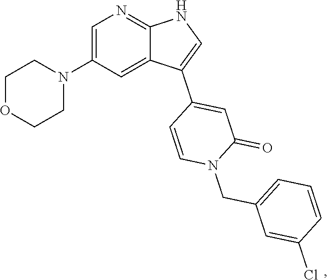 [SMILES: O=c1cc(-c2c[nH]c3ncc(N4CCOCC4)cc23)ccn1Cc1cccc(Cl)c1]

as described in Guichou, J.-F. et al., in WO 2017/085230;

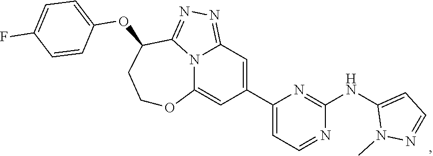 [SMILES: Cn1nccc1Nc1nccc(-c2cc3n4c(nnc4c2)[C@H](Oc2ccc(F)cc2)CCO3)n1]

as described in Kolesnikov, A. et al., in WO 2015/085007;

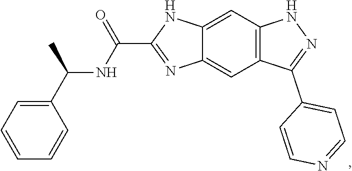 [SMILES: C[C@@H](NC(=O)c1nc2cc3c(-c4ccncc4)n[nH]c3cc2[nH]1)c1ccccc1]

as described in Liu, S. et al., in WO 2019/076336;

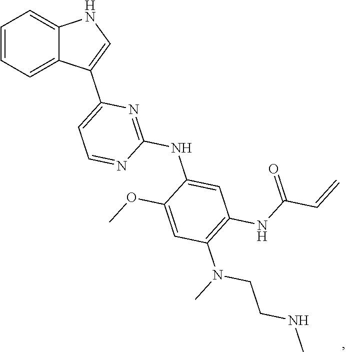 [SMILES: C=CC(=O)Nc1cc(Nc2nccc(-c3c[nH]c4ccccc34)n2)c(OC)cc1N(C)CCNC]

as described in Tang, J. et al., in CN107973783;

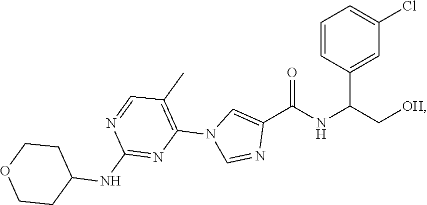 [SMILES: Cc1cnc(NC2CCOCC2)nc1-n1cnc(C(=O)NC(CO)c2cccc(Cl)c2)c1]

as described in Venkatesan, A. M. et al., in US 2016/362406;

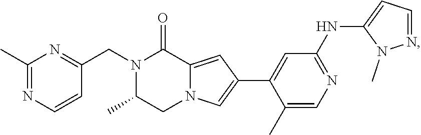 [SMILES: Cc1nccc(CN2C(=O)c3cc(-c4cc(Nc5ccnn5C)ncc4C)cn3C[C@@H]2C)n1]

as described in Ward, R. A. et al., in WO 2017/080980;

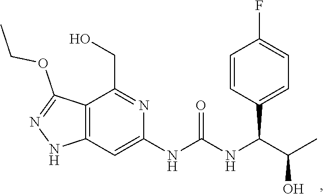 [SMILES: CCOc1n[nH]c2cc(NC(=O)N[C@@H](c3ccc(F)cc3)[C@@H](C)O)nc(CO)c12]

as described in Wilson, K. J. et al., in WO 2014/052566;

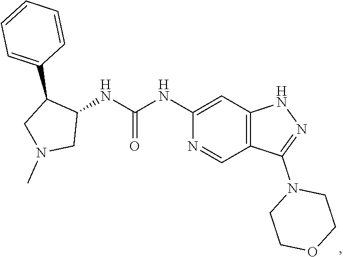 [SMILES: CN1C[C@@H](NC(=O)Nc2cc3[nH]nc(N4CCOCC4)c3cn2)[C@H](c2ccccc2)C1]

as described in Wilson, K. J. et al., in WO 2014/052563;

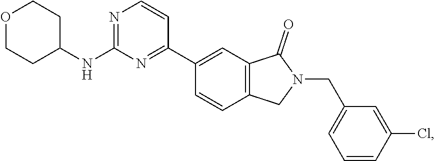 [SMILES: O=C1c2cc(-c3ccnc(NC4CCOCC4)n3)ccc2CN1Cc1cccc(Cl)c1]

as described in Xu, Y. et al., in CN109608444.

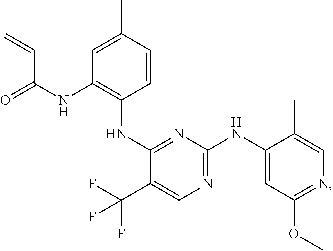 [SMILES: C=CC(=O)Nc1cc(C)ccc1Nc1nc(Nc2cc(OC)ncc2C)ncc1C(F)(F)F]

as describe in Haq, N. et al., in WO2014/124230.

In certain embodiments, the TPL is a radical of one of the above compounds, which is attached to L through a modifiable oxygen, nitrogen, or carbon atom.

### Moiety for FGR

In certain embodiments, the TPL is a moiety that inhibits tyrosine-protein kinase FGR (FGR). Compounds that inhibit FGR are reported in the literature, which include:

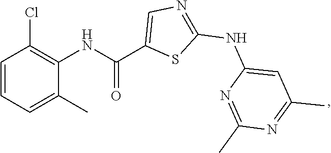 [SMILES: Cc1cc(Nc2ncc(C(=O)Nc3c(C)cccc3Cl)s2)nc(C)n1]

as described in Chen, P. et al., in Bioorg Med Chem Lett 2004, 14(24): 6061.

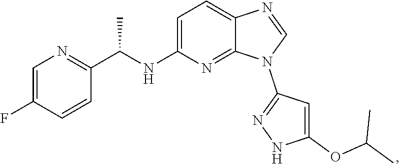 [SMILES: CC(C)Oc1cc(-n2cnc3ccc(N[C@@H](C)c4ccc(F)cn4)nc32)n[nH]1]

as described in Wang, T. et al., in ACS Med Chem Lett 2012, 3(9): 705.

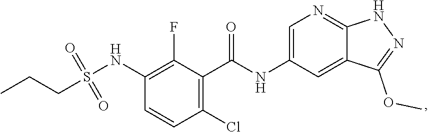 [SMILES: CCCS(=O)(=O)Nc1ccc(Cl)c(C(=O)Nc2cnc3[nH]nc(OC)c3c2)c1F]

as described in Wenglowsky, S. et al., in ACS Med Chem Lett 2011, 2(5): 342.

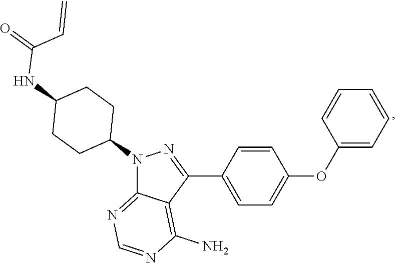 [SMILES: C=CC(=O)N[C@H]1CC[C@@H](n2nc(-c3ccc(Oc4ccccc4)cc3)c3c(N)ncnc32)CC1]

as described in Buggy, J. et al., in US 2012/184567.

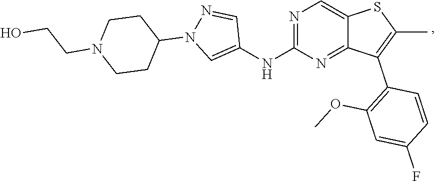 [SMILES: COc1cc(F)ccc1-c1c(C)sc2cnc(Nc3cnn(C4CCN(CCO)CC4)c3)nc12]

as described in Wang, T. et al., in WO 2017/012559.

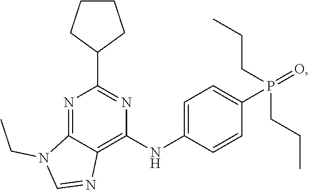 [SMILES: CCCP(=O)(CCC)c1ccc(Nc2nc(C3CCCC3)nc3c2ncn3CC)cc1]

as described in Summy, J. et al., in Mol Cancer Ther 2005, 4(12): 1900.

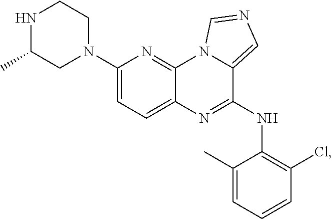 [SMILES: Cc1cccc(Cl)c1Nc1nc2ccc(N3CCN[C@@H](C)C3)nc2n2cncc12]

as described in Chen, P. et al., in J Med Chem 2004, 47(18): 4517.

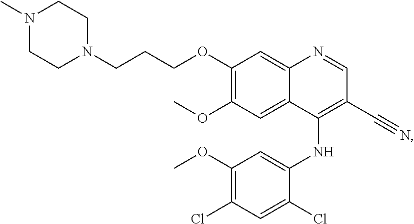 [SMILES: COc1cc(Nc2c(C#N)cnc3cc(OCCCN4CCN(C)CC4)c(OC)cc23)c(Cl)cc1Cl]

as described in Puttini, M. et al., in Cancer Res 2006, 66(23): 11314.

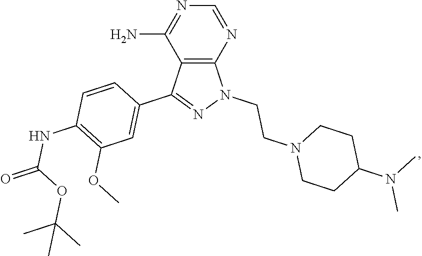 [SMILES: COc1cc(-c2nn(CCN3CCC(N(C)C)CC3)c3ncnc(N)c23)ccc1NC(=O)OC(C)(C)C]

as described in Fraser, C. et al., in J Med Chem 2016, 59(10): 4697.

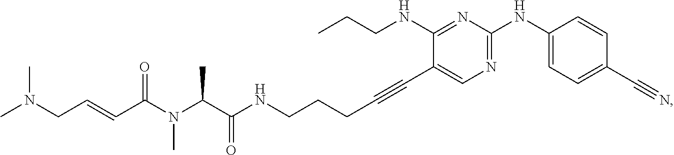 [SMILES: CCCNc1nc(Nc2ccc(C#N)cc2)ncc1C#CCCCNC(=O)[C@H](C)N(C)C(=O)/C=C/CN(C)C]

as described in Yamaura, T. et al., in Blood 2018, 131(4): 426.

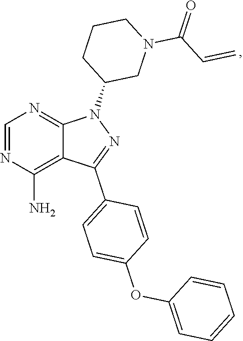 [SMILES: C=CC(=O)N1CCC[C@@H](n2nc(-c3ccc(Oc4ccccc4)cc3)c3c(N)ncnc32)C1]

as described in Wu, H. et al., in ACS Chem Biol 2014, 9(5): 1086.

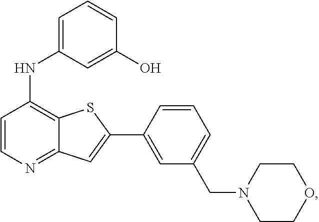 [SMILES: Oc1cccc(Nc2ccnc3cc(-c4cccc(CN5CCOCC5)c4)sc23)c1]

as described in Sun, X. et al., in J Pharmacol Exp Ther 2012, 340(3): 510.

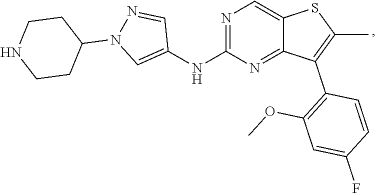 [SMILES: COc1cc(F)ccc1-c1c(C)sc2cnc(Nc3cnn(C4CCNCC4)c3)nc12]

as described in Wang, T. et al., in WO 2017/012559.

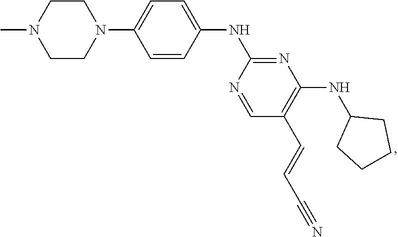 [SMILES: CN1CCN(c2ccc(Nc3ncc(/C=C/C#N)c(NC4CCCC4)n3)cc2)CC1]

as described in Reddy, M. et al., in 255th Am Chem Soc (ACS) Natl Meet⋅2018-03-18/2018-03-22⋅New Orleans, United States⋅Abst MEDI 34.

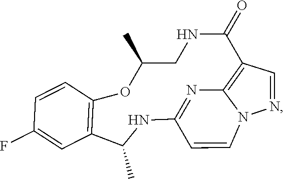 [SMILES: C[C@H]1CNC(=O)c2cnn3ccc(nc23)N[C@H](C)c2cc(F)ccc2O1]

as described in Drilon, A. et al., in Cancer Discov 2018, 8(10): 1227.

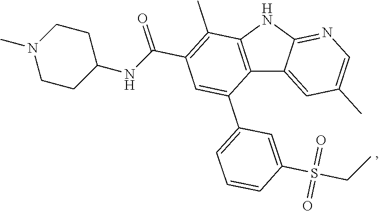 [SMILES: CCS(=O)(=O)c1cccc(-c2cc(C(=O)NC3CCN(C)CC3)c(C)c3[nH]c4ncc(C)cc4c23)c1]

as described in Farrell, P. et al., in Mol Cancer Ther 2013, 12(4): 460.

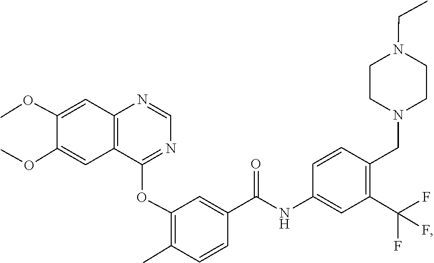 [SMILES: CCN1CCN(Cc2ccc(NC(=O)c3ccc(C)c(Oc4ncnc5cc(OC)c(OC)cc45)c3)cc2C(F)(F)F)CC1]

as described in Weir, M. et al., in ACS Chem Biol 2018, 13(6): 1551.

In certain embodiments, the TPL is a radical of one of the above compounds, which is attached to L through a modifiable oxygen, nitrogen, or carbon atom.

### Moiety for HER3

In certain embodiments, the TPL is a moiety that inhibits receptor tyrosine-protein kinase erbB-3 (HER3). Compounds that inhibit HER3 are reported in the literature, which include:

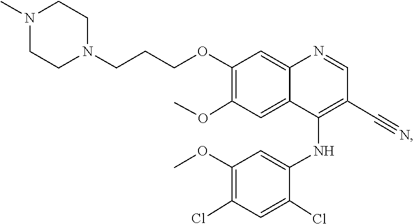 [SMILES: COc1cc(Nc2c(C#N)cnc3cc(OCCCN4CCN(C)CC4)c(OC)cc23)c(Cl)cc1Cl]

as described in Li, L. et al., in Leuk Res 2019, 78: 12.

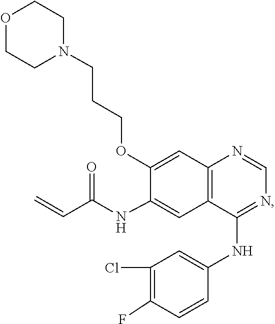 [SMILES: C=CC(=O)Nc1cc2c(Nc3ccc(F)c(Cl)c3)ncnc2cc1OCCCN1CCOCC1]

as described in Allen, L. et al., in Semin Oncol 2002, 29(3, Suppl. 11): 11.

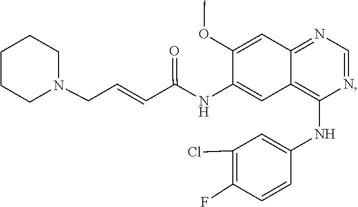 [SMILES: COc1cc2ncnc(Nc3ccc(F)c(Cl)c3)c2cc1NC(=O)/C=C/CN1CCCCC1]

as described in Tecle, H. et al., in US2013274275.

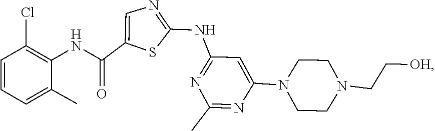 [SMILES: Cc1nc(Nc2ncc(C(=O)Nc3c(C)cccc3Cl)s2)cc(N2CCN(CCO)CC2)n1]

as described in Li, L. et al., in Leuk Res 2019, 78: 12.

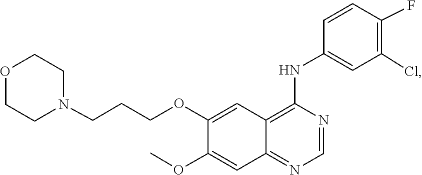 [SMILES: COc1cc2ncnc(Nc3ccc(F)c(Cl)c3)c2cc1OCCCN1CCOCC1]

as described in Marshall, G. et al., in 32nd Annu San Antonio Breast Cancer Symp 2009-12-10/2009-12-13 San Antonio, United States Abst 5059, Cancer Res 2009, 69(24, Suppl. 3).

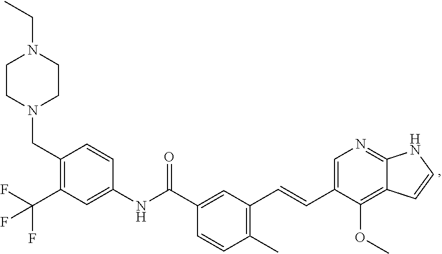 [SMILES: CCN1CCN(Cc2ccc(NC(=O)c3ccc(C)c(/C=C/c4cnc5[nH]ccc5c4OC)c3)cc2C(F)(F)F)CC1]

as described in Tan, L. et al., in J Med Chem 2015, 58(1): 183.

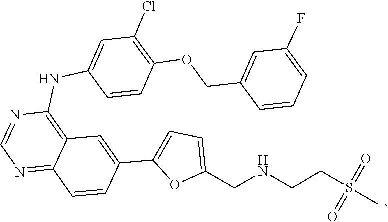 [SMILES: CS(=O)(=O)CCNCc1ccc(-c2ccc3ncnc(Nc4ccc(OCc5cccc(F)c5)c(Cl)c4)c3c2)o1]

as described in Marshall, G. et al., in 32nd Annu San Antonio Breast Cancer Symp⋅2009-12-10/2009-12-13⋅San Antonio, United States⋅Abst 5059, Cancer Res 2009, 69(24, Suppl. 3).

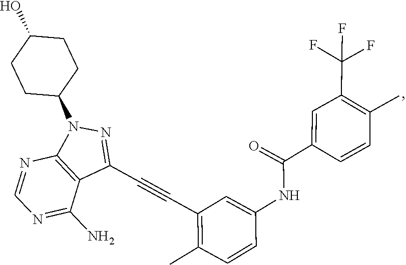 [SMILES: Cc1ccc(NC(=O)c2ccc(C)c(C(F)(F)F)c2)cc1C#Cc1nn([C@H]2CC[C@H](O)CC2)c2ncnc(N)c12]

as described in Zhang, C. et al., in J Med Chem 2016,

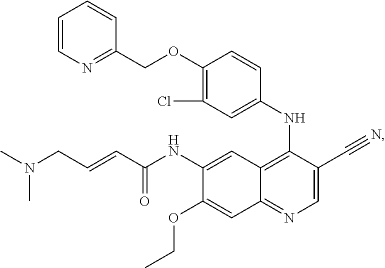 [SMILES: CCOc1cc2ncc(C#N)c(Nc3ccc(OCc4ccccn4)c(Cl)c3)c2cc1NC(=O)/C=C/CN(C)C]

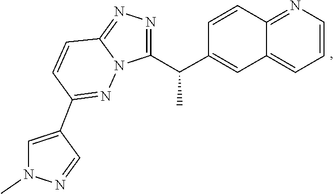 [SMILES: C[C@@H](c1ccc2ncccc2c1)c1nnc2ccc(-c3cnn(C)c3)nn12]

as described in Christensen, G. et al., in Proc Am Assoc Cancer Res (AACR) 2008, 49, Abst.

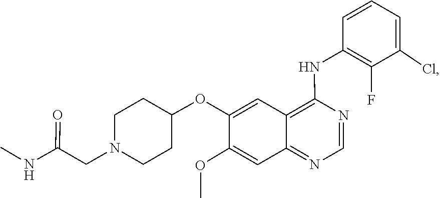 [SMILES: CNC(=O)CN1CCC(Oc2cc3c(Nc4cccc(Cl)c4F)ncnc3cc2OC)CC1]

as described in Dong, X. et al., in Neoplasia 2016, 18(3): 162.

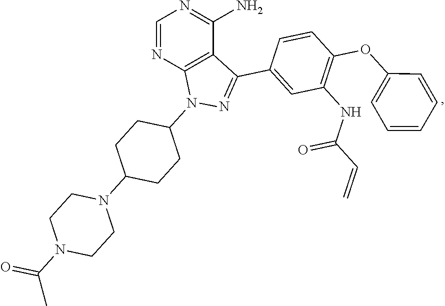 [SMILES: C=CC(=O)Nc1cc(-c2nn(C3CCC(N4CCN(C(C)=O)CC4)CC3)c3ncnc(N)c23)ccc1Oc1ccccc1]

as described in Lim, S. et al., in Bioorg Med Chem Lett 2015, 25(16): 3382.

In certain embodiments, the TPL is a radical of one of the above compounds, which is attached to L through a modifiable oxygen, nitrogen, or carbon atom.

### Moiety for HER4

In certain embodiments, the TPL is a moiety that inhibits receptor tyrosine-protein kinase erbB-4 (HER4). Compounds that inhibit HER4 are reported in the literature, which include:

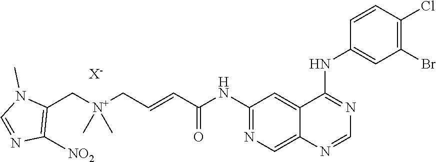

where X is an anion (e.g., Cl⁻), as described in Smaill, J. et al., in WO 2011/028135.

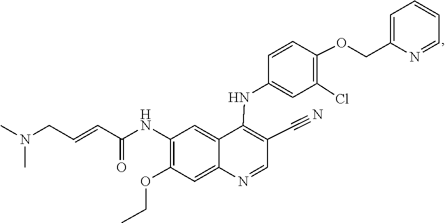 [SMILES: CCOc1cc2ncc(C#N)c(Nc3ccc(OCc4ccccn4)c(Cl)c3)c2cc1NC(=O)/C=C/CN(C)C]

as described in Wissner, A. et al., in WO 2005/028443.

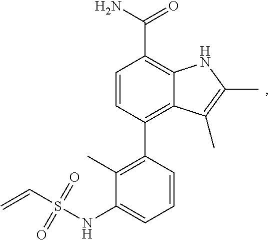 [SMILES: C=CS(=O)(=O)Nc1cccc(-c2ccc(C(N)=O)c3[nH]c(C)c(C)c23)c1C]

as described in Liu, Q. et al., in Bioorg Med Chem Lett 2018, 28(18): 3080.

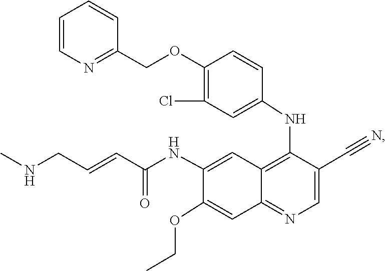 [SMILES: CCOc1cc2ncc(C#N)c(Nc3ccc(OCc4ccccn4)c(Cl)c3)c2cc1NC(=O)/C=C/CNC]

as described in McGinnis, J. et al., in WO 2006/127203.

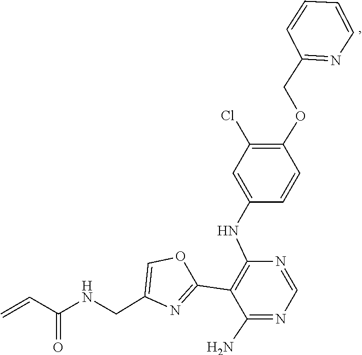 [SMILES: C=CC(=O)NCc1coc(-c2c(N)ncnc2Nc2ccc(OCc3ccccn3)c(Cl)c2)n1]

as described in Cha, M. et al., in J Med Chem 2012, 55(6): 2846.

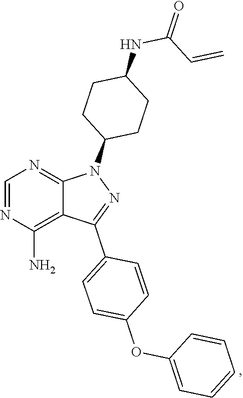 [SMILES: C=CC(=O)N[C@H]1CC[C@@H](n2nc(-c3ccc(Oc4ccccc4)cc3)c3c(N)ncnc32)CC1]

as described in Chen, W. et al., in US 2012/184567.

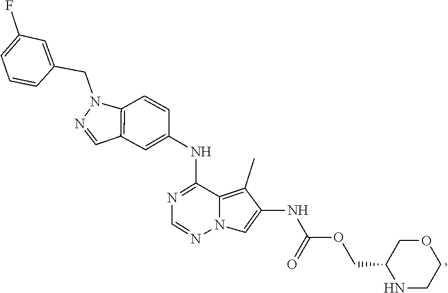 [SMILES: Cc1c(NC(=O)OC[C@@H]2COCCN2)cn2ncnc(Nc3ccc4c(cnn4Cc4cccc(F)c4)c3)c12]

as described in Gavai, A. et al., in J Med Chem 2009, 52(21): 6527.

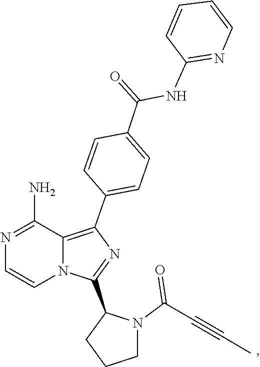 [SMILES: CC#CC(=O)N1CCC[C@H]1c1nc(-c2ccc(C(=O)Nc3ccccn3)cc2)c2c(N)nccn12]

as described in Johnson, D. et al., in WO 2015/110923.

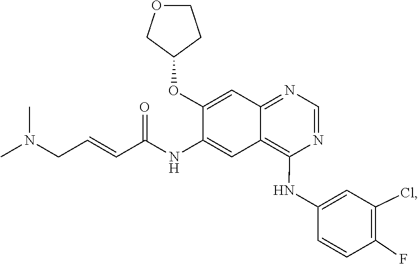 [SMILES: CN(C)C/C=C/C(=O)Nc1cc2c(Nc3ccc(F)c(Cl)c3)ncnc2cc1O[C@H]1CCOC1]

as described in Solca, F. et al., in J Pharmacol Exp Ther 2012, 343(2): 342.

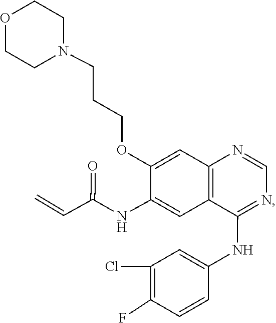 [SMILES: C=CC(=O)Nc1cc2c(Nc3ccc(F)c(Cl)c3)ncnc2cc1OCCCN1CCOCC1]

as described in Solca, F. et al., in J Pharmacol Exp Ther 2012, 343(2): 342.

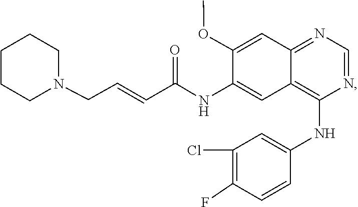 [SMILES: COc1cc2ncnc(Nc3ccc(F)c(Cl)c3)c2cc1NC(=O)/C=C/CN1CCCCC1]

as described in Smaill, J. et al., in J Med Chem 2016, 59(17): 8103.

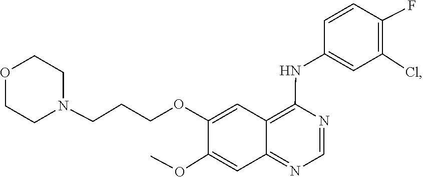 [SMILES: COc1cc2ncnc(Nc3ccc(F)c(Cl)c3)c2cc1OCCCN1CCOCC1]

as described in Wood, E. et al., in Cancer Res 2004, 64(18): 6652.

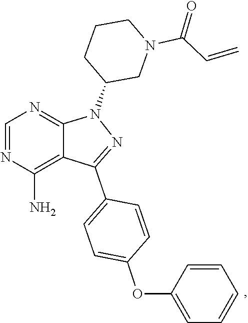

as described in Verner, E. et al., in US2012184013.

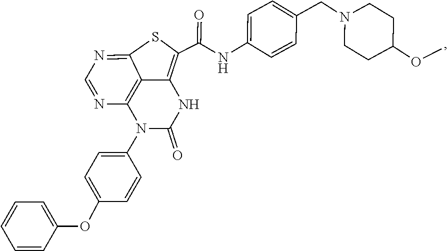 [SMILES: COC1CCN(Cc2ccc(NC(=O)c3sc4ncnc5c4c3NC(=O)N5c3ccc(Oc4ccccc4)cc3)cc2)CC1]

as described in Pandey, N. et al., in Proc Am Assoc Cancer Res (AACR) 2006, 47, Abst 4747.

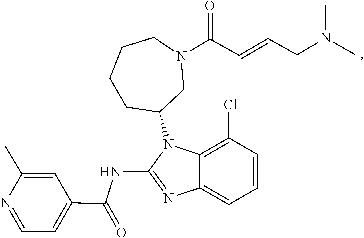

as described in Lelais, G. et al., in J Med Chem 2016, 59(14): 6671.

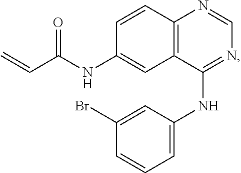 [SMILES: C=CC(=O)Nc1ccc2ncnc(Nc3cccc(Br)c3)c2c1]

as described in Hur, W. et al., in Bioorg Med Chem Lett 2008, 18(22): 5916.

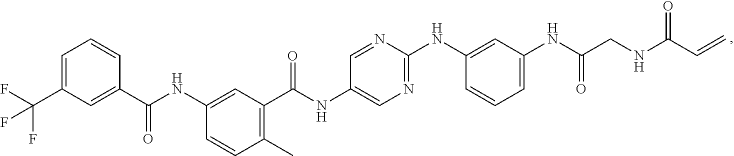 [SMILES: C=CC(=O)NCC(=O)Nc1cccc(Nc2ncc(NC(=O)c3cc(NC(=O)c4cccc(C(F)(F)F)c4)ccc3C)cn2)c1]

as described in Li, X. et al., in J Med Chem 2014, 57(12): 5112.

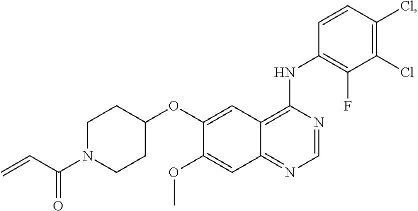 [SMILES: C=CC(=O)N1CCC(Oc2cc3c(Nc4ccc(Cl)c(Cl)c4F)ncnc3cc2OC)CC1]

as described in Cha, M. et al., in Int J Cancer 2012, 130(10): 2445.

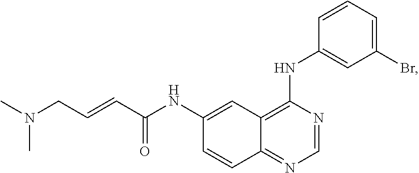 [SMILES: CN(C)C/C=C/C(=O)Nc1ccc2ncnc(Nc3cccc(Br)c3)c2c1]

as described in Smaill, J. et al., in AACR-NCI-EORTC Int Conf Mol Targets Cancer Ther⋅2009-11-15/2009-11-19⋅Boston, United States⋅Abst C46, Mol Cancer Ther 2009, 8(12, Suppl. 1).

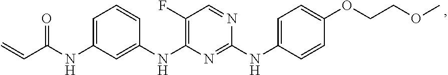 [SMILES: C=CC(=O)Nc1cccc(Nc2nc(Nc3ccc(OCCOC)cc3)ncc2F)c1]

as described in Kaptein, A. et al., in 60th Annu Meet Am Soc Hematol⋅2018-12-01/2018-12-04⋅San Diego, United States⋅Abst 1871, Blood 2018, 132(Suppl. 1).

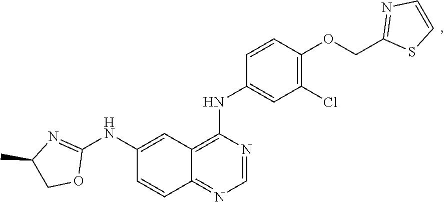 [SMILES: C[C@@H]1COC(Nc2ccc3ncnc(Nc4ccc(OCc5nccs5)c(Cl)c4)c3c2)=N1]

as described in Ooi, A. et al., in 107th Annu Meet Am Assoc Cancer Res (AACR)⋅2016-04-16/2016-04-20⋅New Orleans, United States⋅Abst 4719, Cancer Res 2016, 76(14, Suppl.).

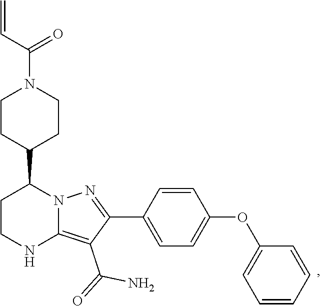 [SMILES: C=CC(=O)N1CCC([C@@H]2CCNc3c(C(N)=O)c(-c4ccc(Oc5ccccc5)cc4)nn32)CC1]

as described in Guo, Y. et al., in J Med Chem 2019, 62(17): 7923.

In certain embodiments, the TPL is a radical of one of the above compounds, which is attached to L through a modifiable oxygen, nitrogen, or carbon atom.

### Moiety for FLT3

In certain embodiments, the TPL is a moiety inhibits receptor-type tyrosine-protein kinase FLT3 (FLT3). Compounds that inhibit FLT3 are reported in the literature, which include:

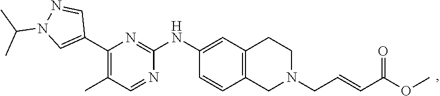 [SMILES: COC(=O)/C=C/CN1CCc2cc(Nc3ncc(C)c(-c4cnn(C(C)C)c4)n3)ccc2C1]

as described in Yang, T. et al., in J Med Chem 2020, 63(23): 14921.

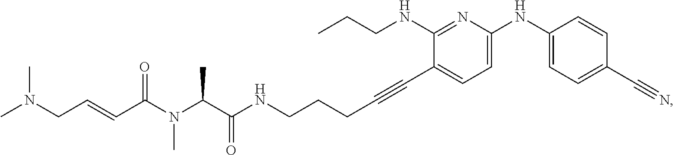

as described in Mizumoto, S. et al., in WO2015056683.

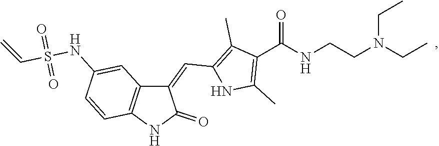 [SMILES: C=CS(=O)(=O)Nc1ccc2c(c1)/C(=C/c1[nH]c(C)c(C(=O)NCCN(CC)CC)c1C)C(=O)N2]

as described in Bensinger, D. et al., in J Med Chem 2019, 62(5): 2428.

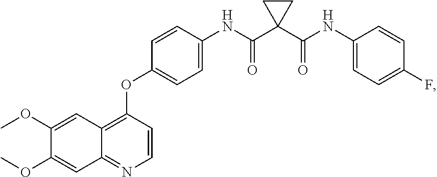 [SMILES: COc1cc2nccc(Oc3ccc(NC(=O)C4(C(=O)Nc5ccc(F)cc5)CC4)cc3)c2cc1OC]

as described in Xu, Q. et al., in Bioorg Med Chem Lett 2019, 29(19), 126630.

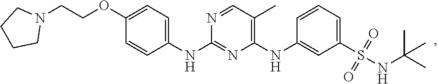 [SMILES: Cc1cnc(Nc2ccc(OCCN3CCCC3)cc2)nc1Nc1cccc(S(=O)(=O)NC(C)(C)C)c1]

as described in Wan, H. et al., in ACS Med Chem Lett 2015, 6(8): 850.

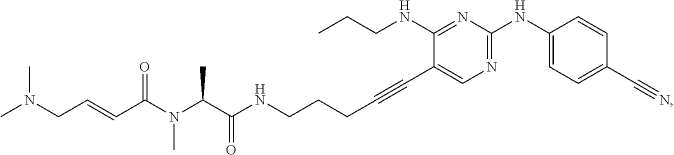 [SMILES: CCCNc1nc(Nc2ccc(C#N)cc2)ncc1C#CCCCNC(=O)[C@H](C)N(C)C(=O)/C=C/CN(C)C]

as described in Nakatani, T. et al., in 57th Annu Meet Am Soc Hematol⋅2015-12-05/2015-12-08⋅Orlando, United States⋅Abst 1353, Blood 2015, 126(51).

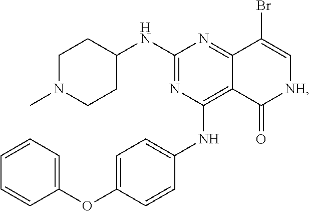 [SMILES: CN1CCC(Nc2nc(Nc3ccc(Oc4ccccc4)cc3)c3c(=O)[nH]cc(Br)c3n2)CC1]

as described in Xin, C. et al., in 110th Annu Meet Am Assoc Cancer Res (AACR)⋅2019-03-29/2019-04-03⋅Atlanta, United States⋅Abst 2010, Cancer Res 2019, 79(13, Suppl.).

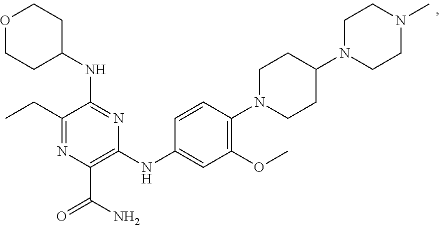 [SMILES: CCc1nc(C(N)=O)c(Nc2ccc(N3CCC(N4CCN(C)CC4)CC3)c(OC)c2)nc1NC1CCOCC1]

as described in Jeong, P. et al., in Eur J Med Chem 2020, 195: 112205.

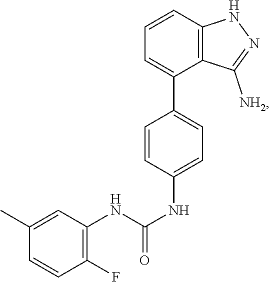 [SMILES: Cc1ccc(F)c(NC(=O)Nc2ccc(-c3cccc4[nH]nc(N)c34)cc2)c1]

as described in Li, J. et al., in Proc Am Assoc Cancer Res (AACR) 2005, 46, Abst 5981.

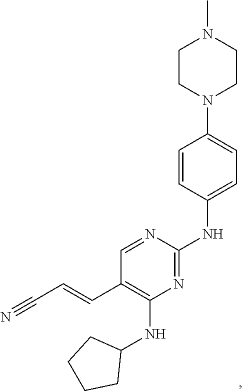 [SMILES: CN1CCN(c2ccc(Nc3ncc(/C=C/C#N)c(NC4CCCC4)n3)cc2)CC1]

as described in Reddy, M. et al., in 255th Am Chem Soc (ACS) Natl Meet⋅2018-03-18/2018-03-22⋅New Orleans, United States⋅Abst MEDI 34.

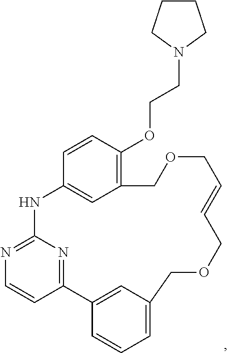 [SMILES: C1=C/COCc2cc(ccc2OCCN2CCCC2)Nc2nccc(n2)-c2cccc(c2)COC/1]

as described in William, A. et al., in J Med Chem 2011, 54(13): 4638.

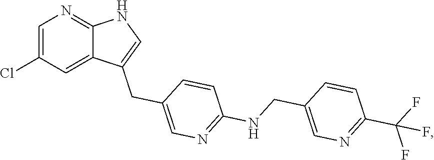 [SMILES: FC(F)(F)c1ccc(CNc2ccc(Cc3c[nH]c4ncc(Cl)cc34)cn2)cn1]

as described in Smith, C. et al., in Cancer Discov 2015, 5(6): 668.

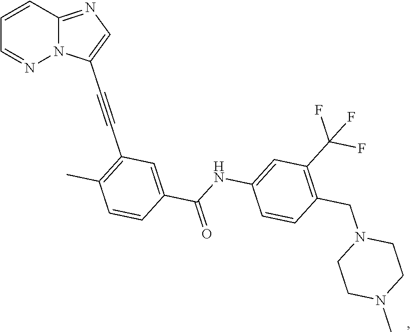 [SMILES: Cc1ccc(C(=O)Nc2ccc(CN3CCN(C)CC3)c(C(F)(F)F)c2)cc1C#Cc1cnc2cccnn12]

as described in Gozgit, J. et al., in Mol Cancer Ther 2011, 10(6): 1028.

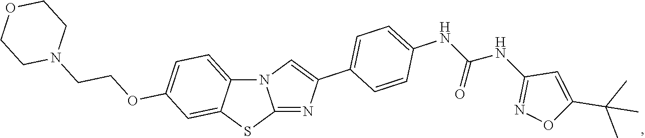 [SMILES: CC(C)(C)c1cc(NC(=O)Nc2ccc(-c3cn4c(n3)sc3cc(OCCN5CCOCC5)ccc34)cc2)no1]

as described in Galanis, A. et al., in 103rd Annu Meet Am Assoc Cancer Res (AACR)⋅2012-03-31/2012-04-04⋅Chicago, United States⋅Abst 3660, Cancer Res 2012, 72(Suppl. 8).

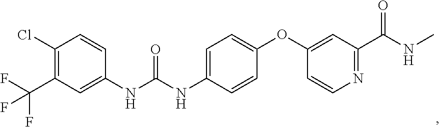 [SMILES: CNC(=O)c1cc(Oc2ccc(NC(=O)Nc3ccc(Cl)c(C(F)(F)F)c3)cc2)ccn1]

as described in Auclair, D. et al., in Proc Am Assoc Cancer Res (AACR) 2005, 46, Abst 5991.

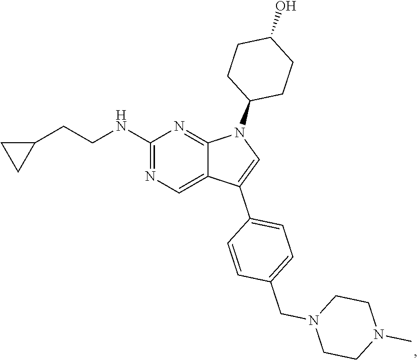 [SMILES: CN1CCN(Cc2ccc(-c3cn([C@H]4CC[C@H](O)CC4)c4nc(NCCC5CC5)ncc34)cc2)CC1]

as described in Minson, K. et al., in 56th Annu Meet Am Soc Hematol⋅2014-12-06/2014-12-09⋅San Francisco, United States⋅Abst 3757.

In certain embodiments, the TPL is a radical of one of the above compounds, which is attached to L through a modifiable oxygen, nitrogen, or carbon atom.

### Moiety for IDH1

In certain embodiments, the TPL is a moiety that binds to and/or inhibits Isocitrate dehydrogenase 1 (IDH1). Compounds that bind and/or inhibit IDH1 are reported in the literature, which include ivosidenib (AG-120), AG-120 (racemic), vorasidenib (AG-881), and BAY 1436032. Additional exemplary compounds that inhibit and/or bind IDH1 are:

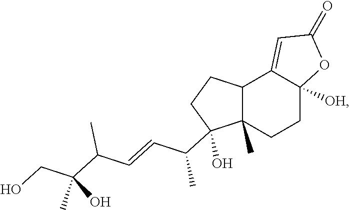 [SMILES: CC(/C=C/[C@@H](C)[C@@]1(O)CCC2C3=CC(=O)O[C@@]3(O)CC[C@@]21C)[C@@](C)(O)CO]

as described by Chen, L. et al., in CN108440471.

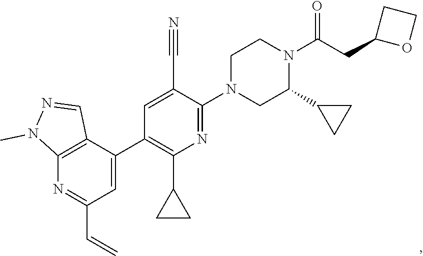 [SMILES: C=Cc1cc(-c2cc(C#N)c(N3CCN(C(=O)C[C@H]4CCO4)[C@H](C4CC4)C3)nc2C2CC2)c2cnn(C)c2n1]

as described by Cao, S. et al., in WO 2012171337.

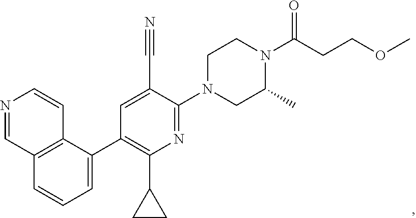 [SMILES: COCCC(=O)N1CCN(c2nc(C3CC3)c(-c3cccc4cnccc34)cc2C#N)C[C@H]1C]

as described by Cao, S. et al., in WO 2012171337.

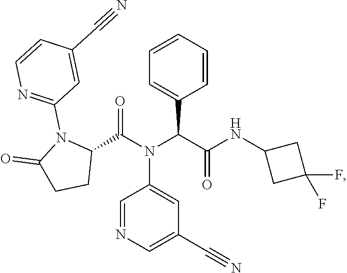 [SMILES: N#Cc1cncc(N(C(=O)[C@@H]2CCC(=O)N2c2cc(C#N)ccn2)[C@H](C(=O)NC2CC(F)(F)C2)c2ccccc2)c1]

as described by Zhou, D. et al., in WO 2015010626.

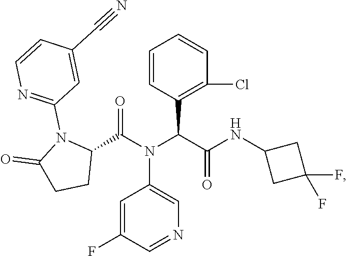 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

as described by Lemieux, R. M. et al., in CN106496090.

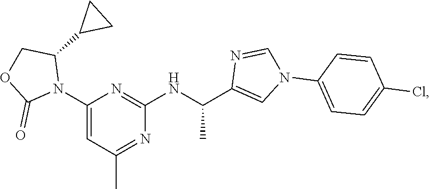 [SMILES: Cc1cc(N2C(=O)OC[C@@H]2C2CC2)nc(N[C@@H](C)c2cn(-c3ccc(Cl)cc3)cn2)n1]

as described by Shultz, M. D. et al., in WO 2014141153.

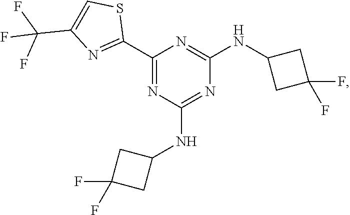 [SMILES: FC1(F)CC(Nc2nc(NC3CC(F)(F)C3)nc(-c3nc(C(F)(F)F)cs3)n2)C1]

as described by Konteatis, Z. D. et al., in WO 2015003640.

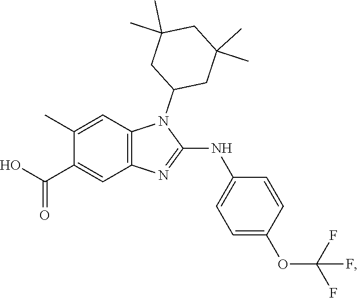 [SMILES: Cc1cc2c(cc1C(=O)O)nc(Nc1ccc(OC(F)(F)F)cc1)n2C1CC(C)(C)CC(C)(C)C1]

as described by Zimmermann, K. et al., in WO 2015121209.

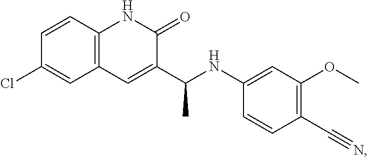 [SMILES: COc1cc(N[C@@H](C)c2cc3cc(Cl)ccc3[nH]c2=O)ccc1C#N]

as described by Lin, J. et al., in US 2018327361.

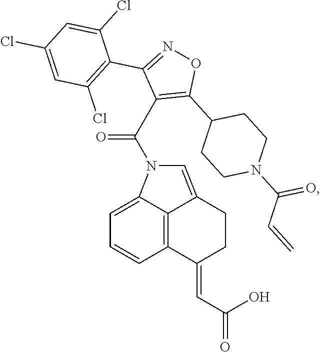 [SMILES: C=CC(=O)N1CCC(c2onc(-c3c(Cl)cc(Cl)cc3Cl)c2C(=O)n2cc3c4c(cccc42)/C(=C/C(=O)O)CC3)CC1]

as described by Matsunag, H. et al., in US 20162052.

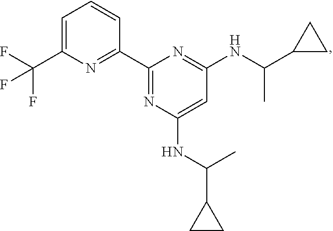 [SMILES: CC(Nc1cc(NC(C)C2CC2)nc(-c2cccc(C(F)(F)F)n2)n1)C1CC1]

as described by Konteaag, H. et al., in US 201620529.

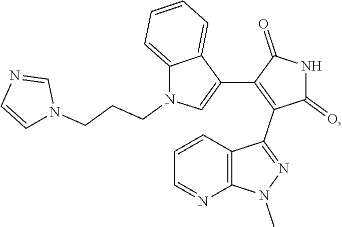 [SMILES: Cn1nc(C2=C(c3cn(CCCn4ccnc4)c4ccccc34)C(=O)NC2=O)c2cccnc21]

as described by Ye, Q. et al., in CN109535158.

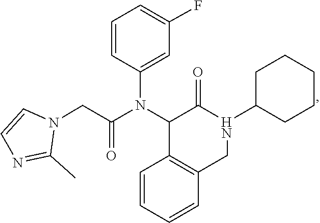 [SMILES: Cc1nccn1CC(=O)N(c1cccc(F)c1)C1C(=O)N(C2CCCCC2)Cc2ccccc21]

as described by Ye, Q. et al., in CN109535158.

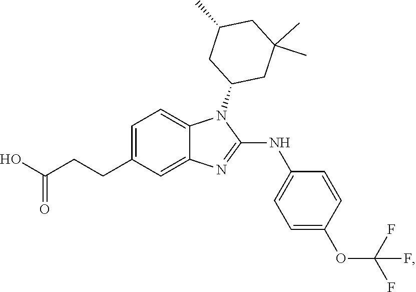 [SMILES: C[C@H]1C[C@@H](n2c(Nc3ccc(OC(F)(F)F)cc3)nc3cc(CCC(=O)O)ccc32)CC(C)(C)C1]

as described by Schirmer, H. et al., in WO 2017016992.

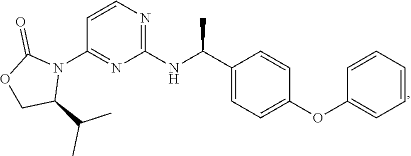 [SMILES: CC(C)[C@H]1COC(=O)N1c1ccnc(N[C@@H](C)c2ccc(Oc3ccccc3)cc2)n1]

as described by Sutton, J. et al., in WO 2013046136.

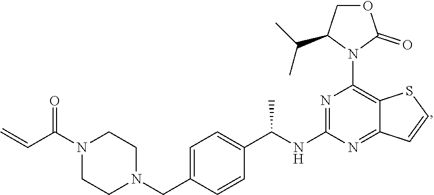 [SMILES: C=CC(=O)N1CCN(Cc2ccc([C@H](C)Nc3nc(N4C(=O)OC[C@@H]4C(C)C)c4sccc4n3)cc2)CC1]

as described by Zhang, T. et al., in WO 2018118793.

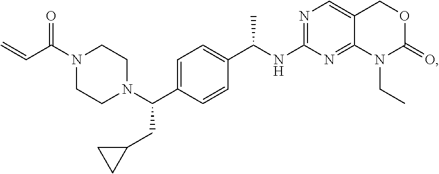 [SMILES: C=CC(=O)N1CCN([C@@H](CC2CC2)c2ccc([C@H](C)Nc3ncc4c(n3)N(CC)C(=O)OC4)cc2)CC1]

as described by Hahn, P. J. et al., in WO 2018111707.

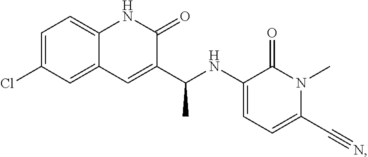 [SMILES: C[C@H](Nc1ccc(C#N)n(C)c1=O)c1cc2cc(Cl)ccc2[nH]c1=O]

as described by Lin, J. et al., in US 2019/0263778.

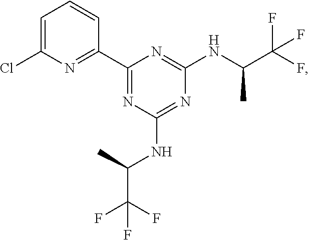 [SMILES: C[C@@H](Nc1nc(N[C@H](C)C(F)(F)F)nc(-c2cccc(Cl)n2)n1)C(F)(F)F]

as described by Cai, Z. et al., in WO 2015003640.

In certain embodiments, the TPL is a radical of one of the above compounds, which is attached to L through a modifiable oxygen, nitrogen, or carbon atom.

### Moiety for HCV NS3 Protease

In certain embodiments, the TPL is a moiety that binds to and/or inhibits Hepatitis C NS3 Protease (HCV NS3 Protease). Compounds that bind and/or inhibit HCV NS3 Protease are reported in the literature, which include:

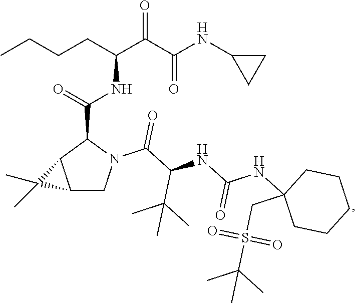 [SMILES: CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

as described in Bennett, F. et al., in WO 2005/087731.

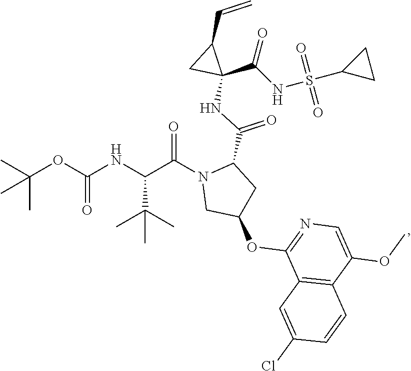 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

as described in Sit, S.-Y. et al., in WO 2003/099274.

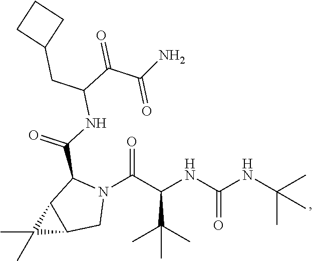 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

as described in Tamura, S. et al., in WO 2002/008244.

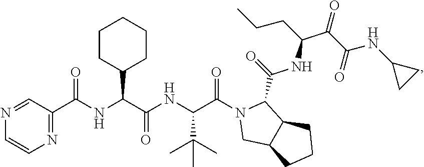 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

as described in Victor, F. et al., in WO 20020/18369.

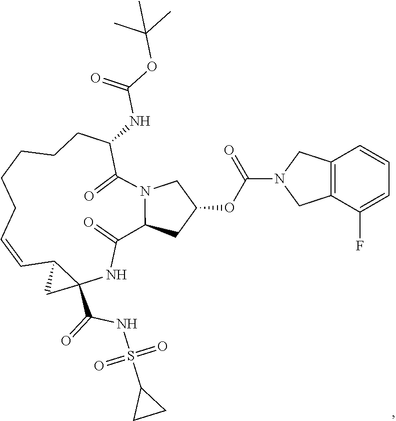 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]

as described in Blatt, L. et al., in WO 2005/037214.

In certain embodiments, the TPL is a radical of one of the above compounds, which is attached to L through a modifiable oxygen, nitrogen, or carbon atom.

### Moiety for PI3Kα

In certain embodiments, the TPL is a moiety that is an inhibitor of phosphoinositide 3-kinase-alpha (PI3Kα). Compounds that inhibitor PI3Kα are reported in the literature, which include:

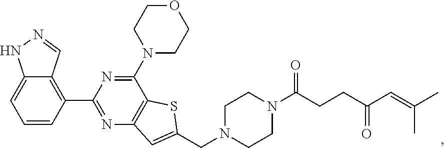 [SMILES: CC(C)=CC(=O)CCC(=O)N1CCN(Cc2cc3nc(-c4cccc5[nH]ncc45)nc(N4CCOCC4)c3s2)CC1]

as described in Niu, D. et al., in WO 2011/031896.

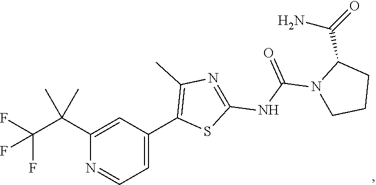 [SMILES: Cc1nc(NC(=O)N2CCC[C@H]2C(N)=O)sc1-c1ccnc(C(C)(C)C(F)(F)F)c1]

as described in Caravatti, G. et al., in WO 2010/029082.

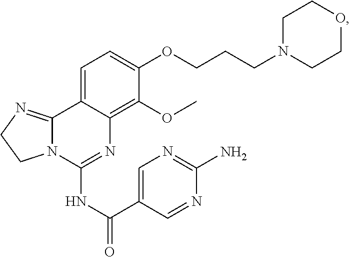 [SMILES: COc1c(OCCCN2CCOCC2)ccc2c1N=C(NC(=O)c1cnc(N)nc1)N1CCN=C21]

as described in Hentemann, M. et al., in WO 2008/070150.

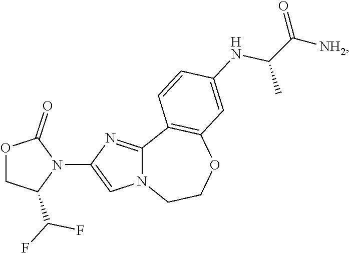 [SMILES: C[C@H](Nc1ccc2c(c1)OCCn1cc(N3C(=O)OC[C@H]3C(F)F)nc1-2)C(N)=O]

as described in Braun, M. et al., in WO 2017/001645.

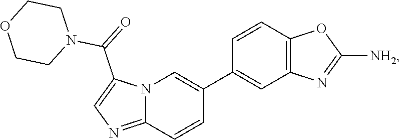 [SMILES: Nc1nc2cc(-c3ccc4ncc(C(=O)N5CCOCC5)n4c3)ccc2o1]

as described in Ren, P. et al., in WO 2011/022439.

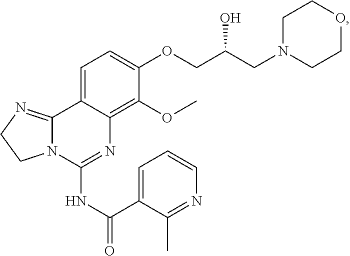 [SMILES: COc1c(OC[C@H](O)CN2CCOCC2)ccc2c1N=C(NC(=O)c1cccnc1C)N1CCN=C21]

as described in Scott, W. et al., in WO 2012/062748.

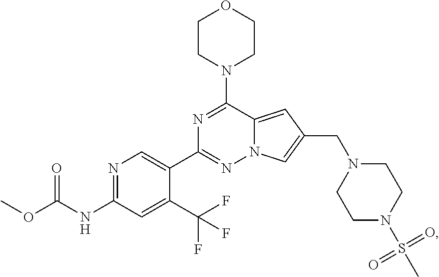 [SMILES: COC(=O)Nc1cc(C(F)(F)F)c(-c2nc(N3CCOCC3)c3cc(CN4CCN(S(C)(=O)=O)CC4)cn3n2)cn1]

as described in Yang, C. et al., in WO 2013/177983.

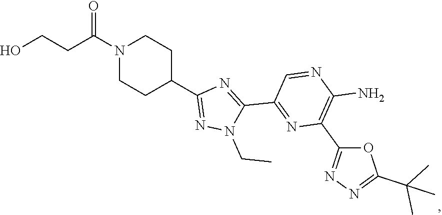 [SMILES: CCn1nc(C2CCN(C(=O)CCO)CC2)nc1-c1cnc(N)c(-c2nnc(C(C)(C)C)o2)n1]

as described in Baarlaam, B. et al., in WO 2014/114928.

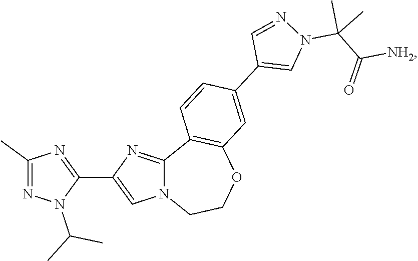 [SMILES: Cc1nc(-c2cn3c(n2)-c2ccc(-c4cnn(C(C)(C)C(N)=O)c4)cc2OCC3)n(C(C)C)n1]

as described in Blaquiere, N. et al., in WO 2011/036280.

In certain embodiments, the TPL is a radical of one of the above compounds, which is attached to L through a modifiable oxygen, nitrogen, or carbon atom.

In certain embodiments, the TPL is selected from those depicted in the compounds in Tables 3 and 3A, below.

### Additional Features

Certain embodiments above describe compounds and/or moieties that contain a warhead (WH) group. In certain embodiments, the WH group is R^{WH}, which is an electrophilic group capable of reacting with a protein, such as reacting with a nucleophilic functional group of a protein, such as a sulfhydryl group of a cysteine residue or an amino group of a lysine residue.

In certain embodiments, R^{WH} is -L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂); and
Y^{W} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted.

In certain embodiments, R^{WH} is -L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂)—; and
Y^{W} is

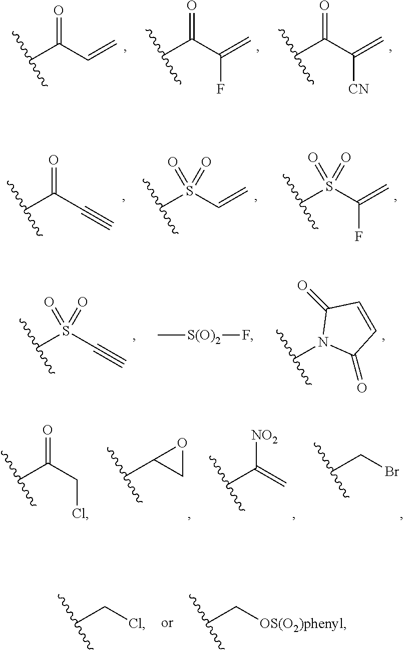

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted. In certain embodiments, R^{WH} is

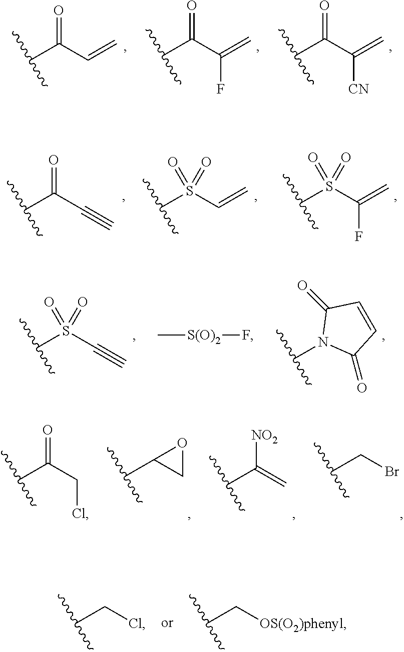

each of which is optionally substituted. In certain embodiments, R^{W} is

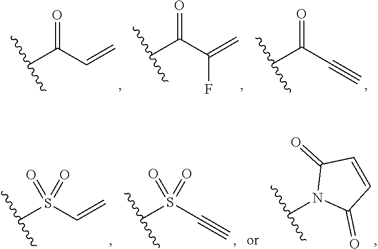

each of which is optionally substituted. In certain embodiments, R^{WH} is

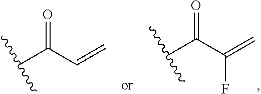

each of which is optionally substituted. In certain embodiments, R^{WH} is

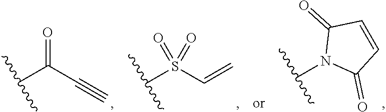

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl. In certain embodiments, R^{WH} is

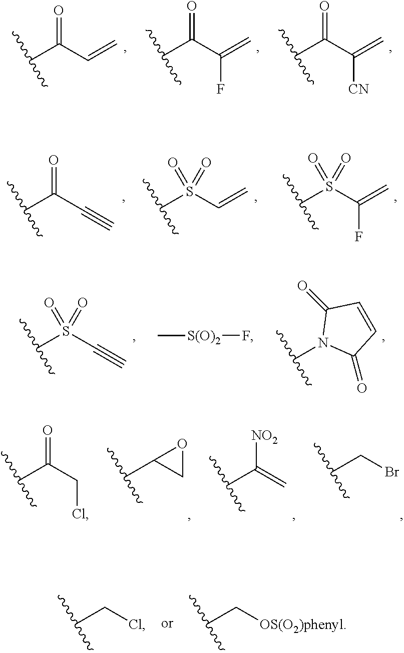

In certain embodiments, R^{WH} is

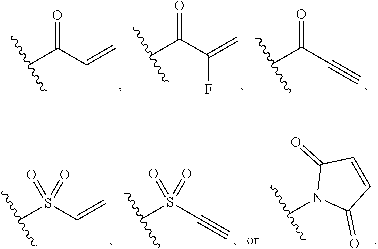

In certain embodiments, R^{WH} is

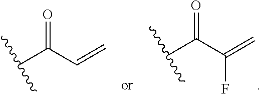

In certain embodiments, R^{W} is

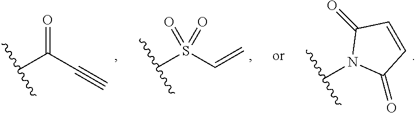

Compounds of Formula I and/or Formula II may be further characterized according to the molecular weight of the TPL. In certain embodiments, the TPL has a molecular weight of less than 1500 Da, 1200 Da, 1000 Da, 800 Da, 600 Da, 400 Da, 300 Da, 200 Da, 150 Da, or 100 Da. Compounds of Formula II may be further characterized according to the molecular weight of the EPL. In certain embodiments, the EPL has a molecular weight of less than 1500 Da, 1200 Da, 1000 Da, 800 Da, 600 Da, 400 Da, 300 Da, 200 Da, 150 Da, or 100 Da.

### Part D: Exemplary Further Description of Linker (L) Component of Compounds of Formula I and II

Compounds of Formula I and II may be further characterized according to, for example, the identity of the linker (L) component. A variety of linkers are known to one of skill in the art and may be used in the heterobifunctional compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, L is symmetrical. In some embodiments, L is asymmetric. In certain embodiments, L is a bond.

In certain embodiments, L is a covalent bond or a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein (i) 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, and (ii) 0-1 methylene units of the hydrocarbon are independently replaced with —C(O)—(C₂₋₆ alkenylene)-, —C(O)—(C₂₋₆ fluoroalkenylene)-, —C(O)—(C₂₋₆ alkynylene)-, —S(O)₂—(C₂₋₆ alkenylene)-, —S(O)₂—(C₂₋₆ fluoroalkenylene)-, —S(O)₂—(C₂₋₆ alkynylene)-, or —(C₁₋₆ alkylene substituted with one R^{WH})—, wherein R^{WH} is

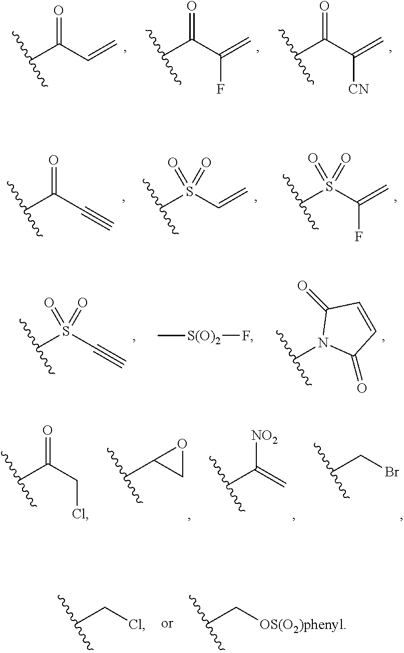

In yet other embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, L is a heteroalkylene having from 4 to 10 atoms selected from carbon, oxygen, nitrogen, and sulfur. In certain embodiments, L is a heteroalkylene having from 4 to 30 atoms selected from carbon, oxygen, and nitrogen. In certain embodiments, L is a heteroalkylene having from 4 to 20 atoms selected from carbon, oxygen, and nitrogen. In certain embodiments, L is a heteroalkylene having from 4 to 10 atoms selected from carbon, oxygen, and nitrogen. In certain embodiments, L is a heteroalkylene having from 4 to 30 atoms selected from carbon and oxygen. In certain embodiments, L is a heteroalkylene having from 4 to 20 atoms selected from carbon and oxygen. In certain embodiments, L is a heteroalkylene having from 4 to 10 atoms selected from carbon and oxygen.

In additional embodiments, the L is an optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, L is substituted with an aryl, phenyl, benzyl, alkyl, alkylene, or heterocycle group.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, L has the formula —N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In some embodiments, L is one of the following:

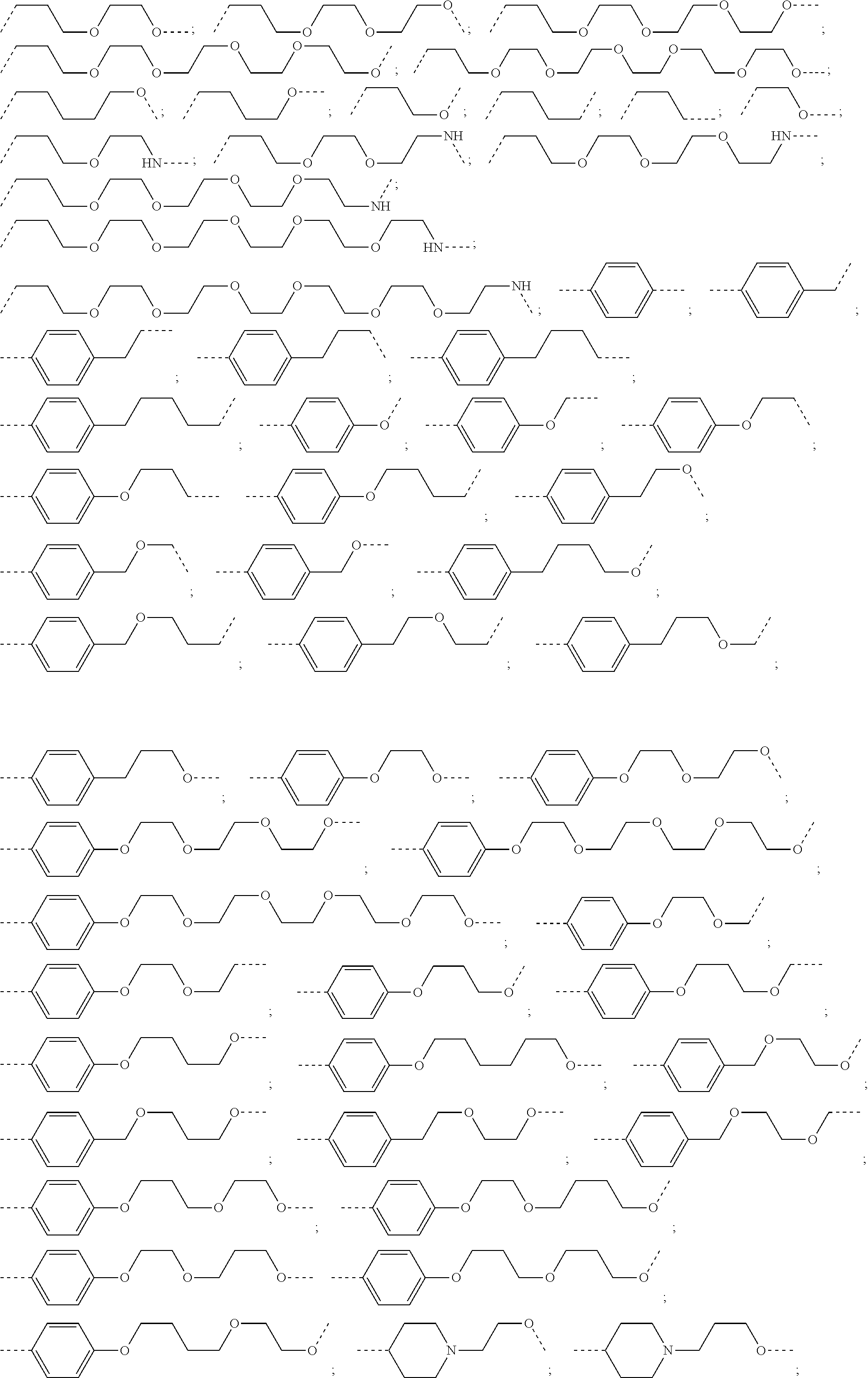

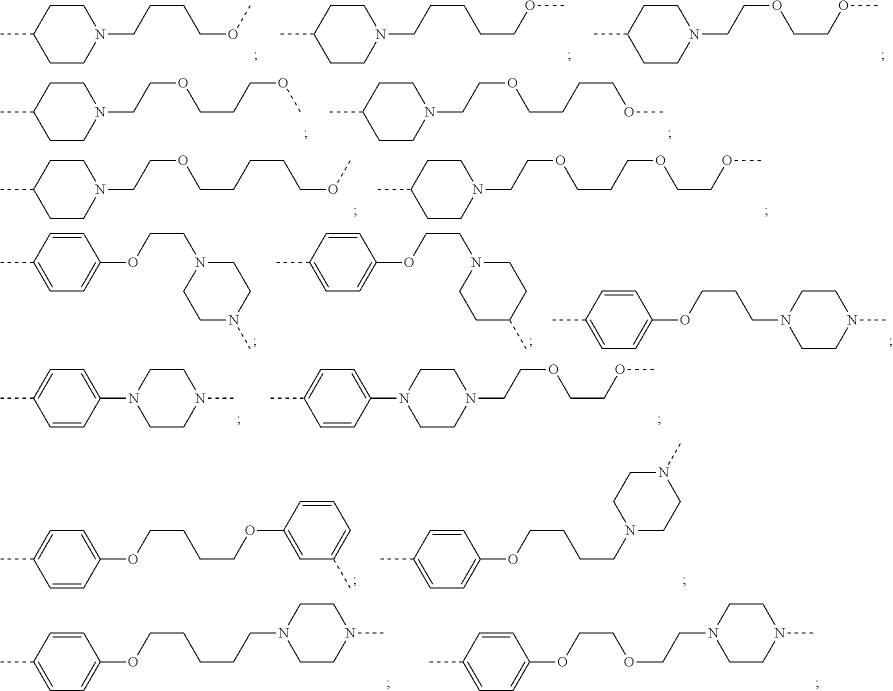

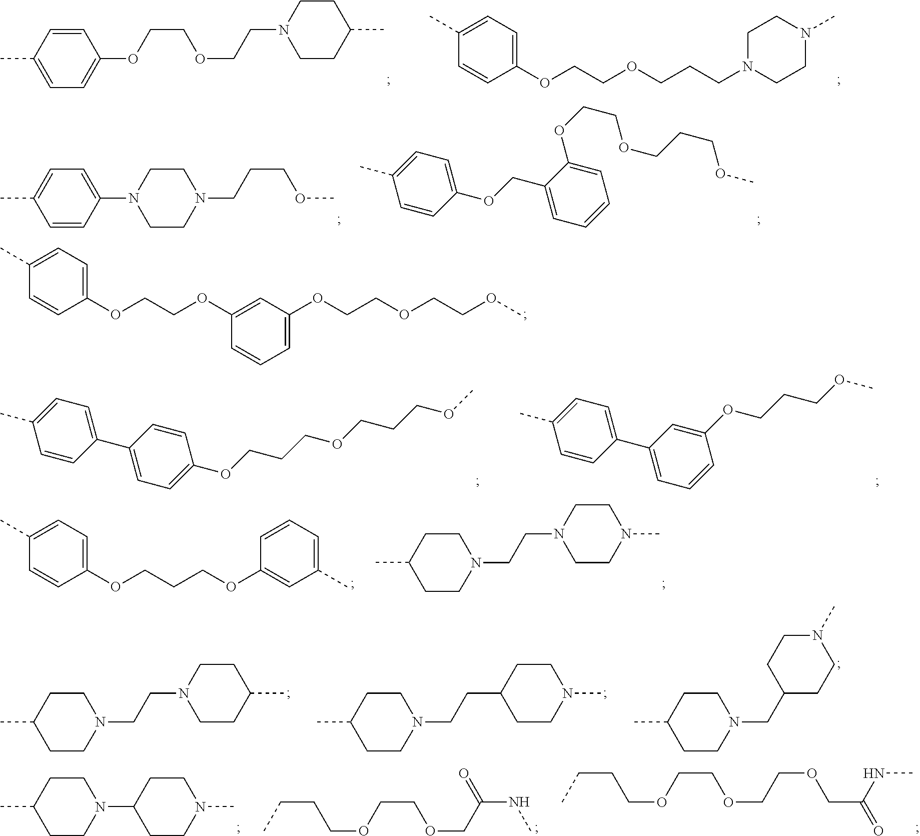

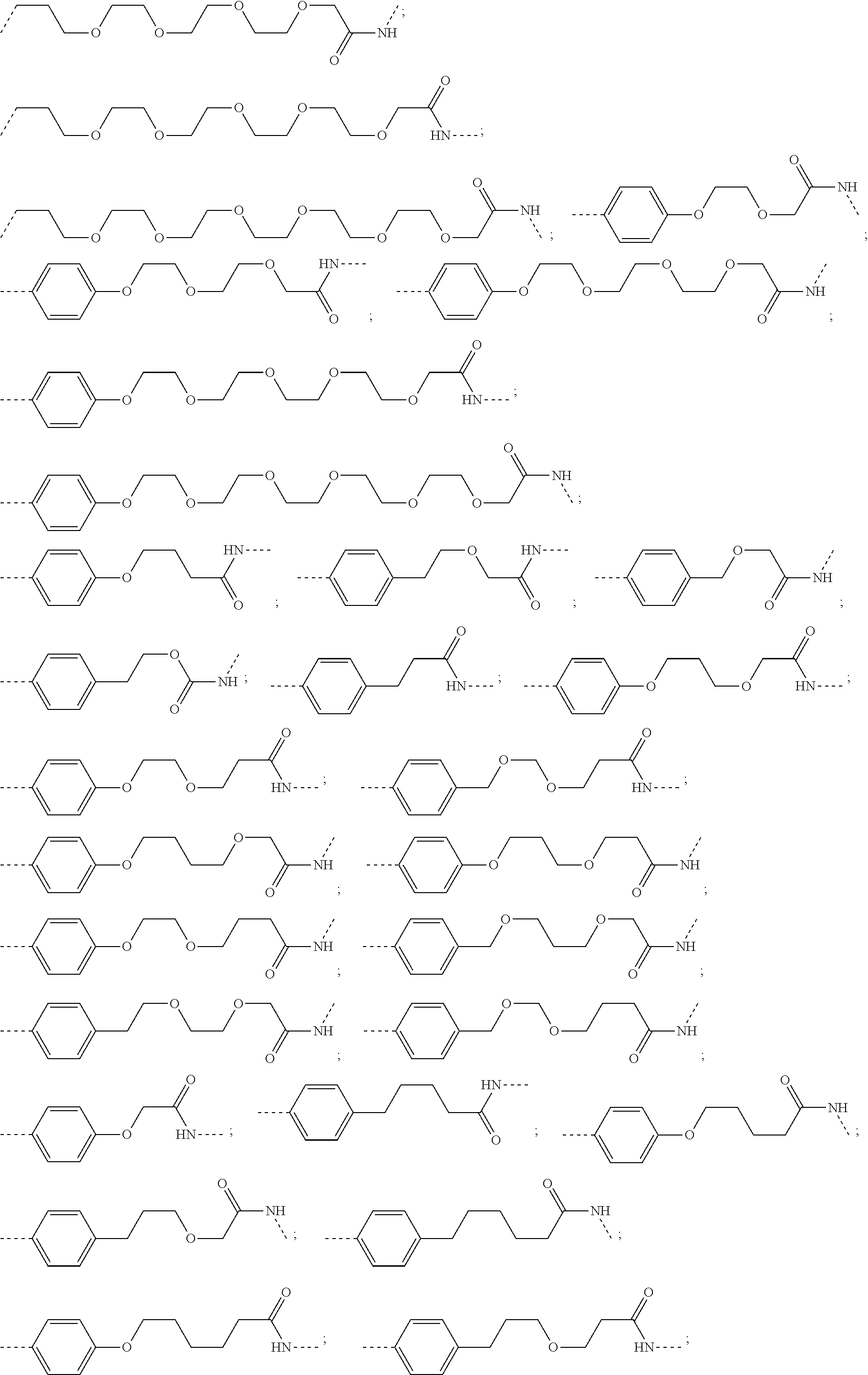

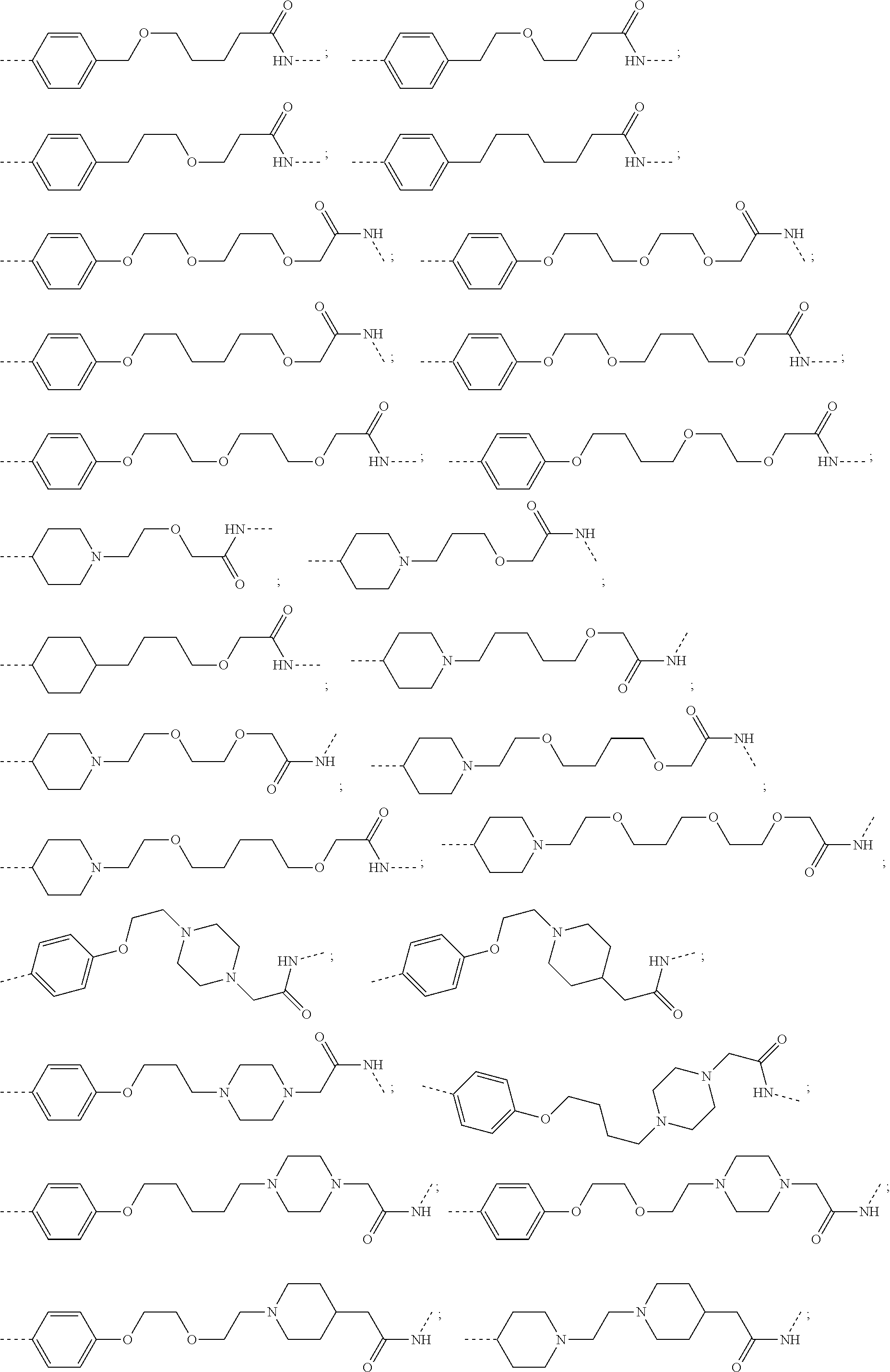

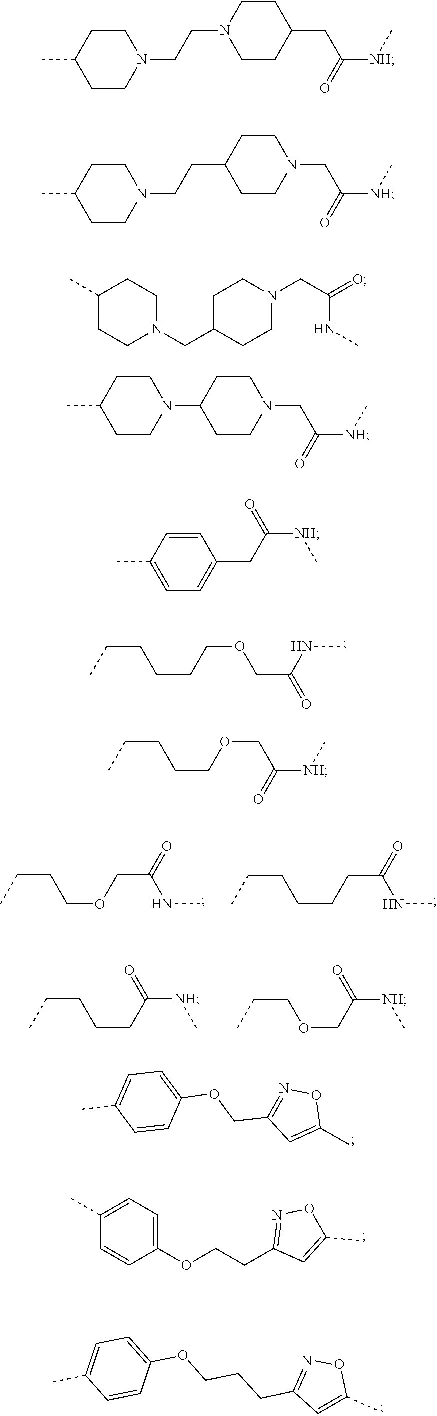

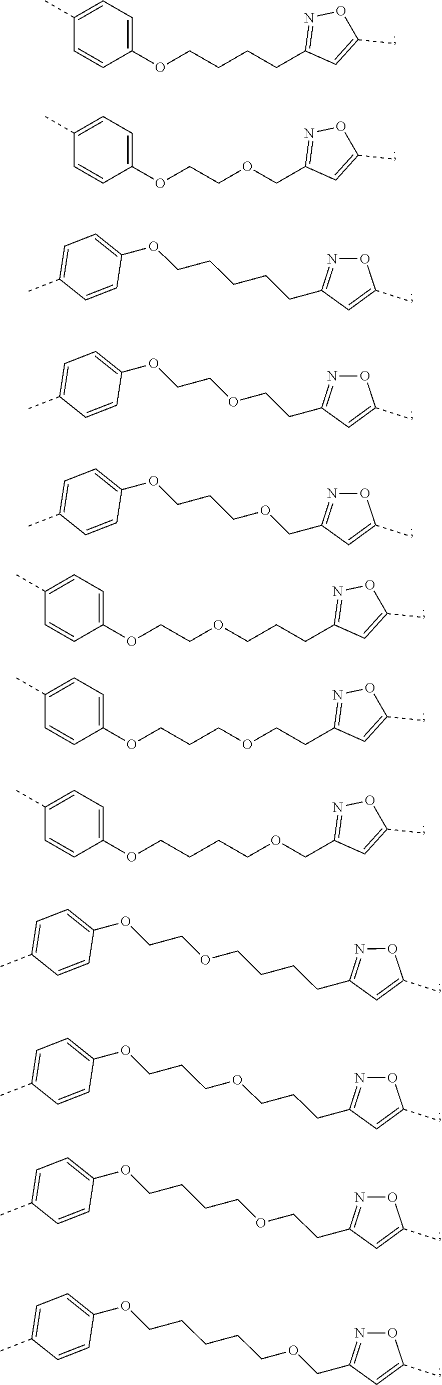

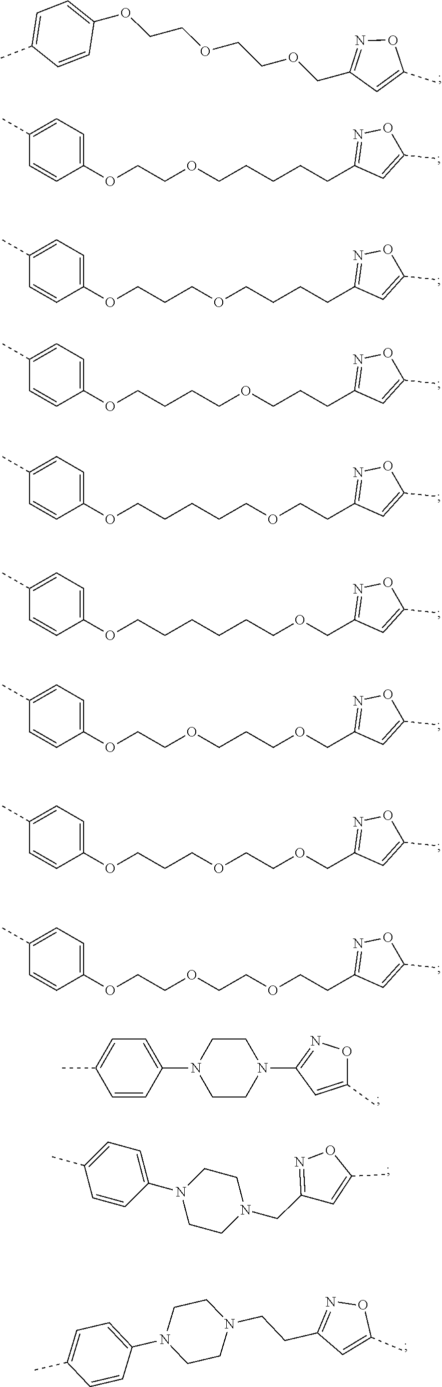

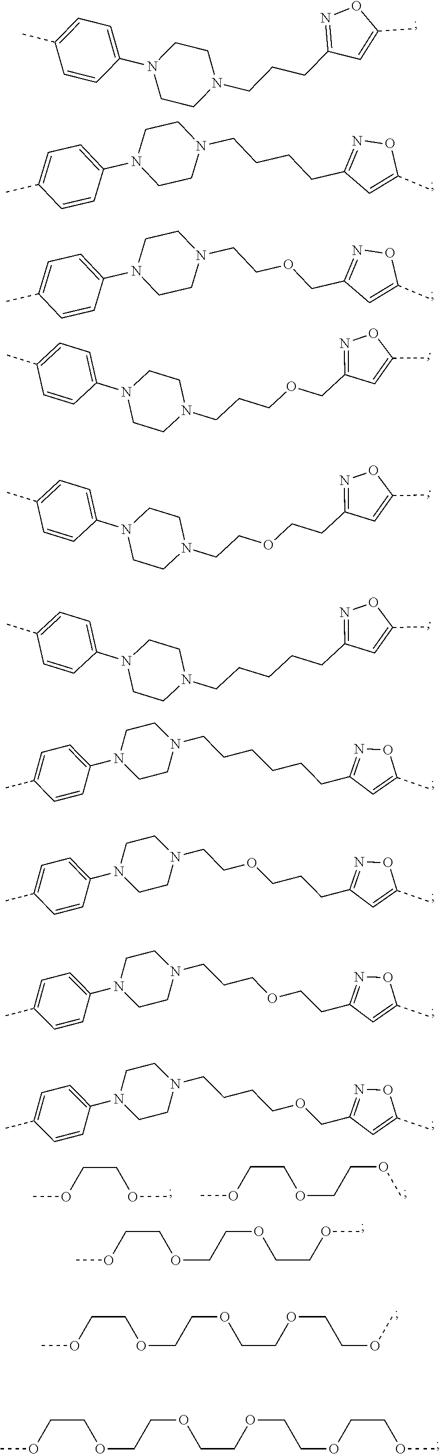

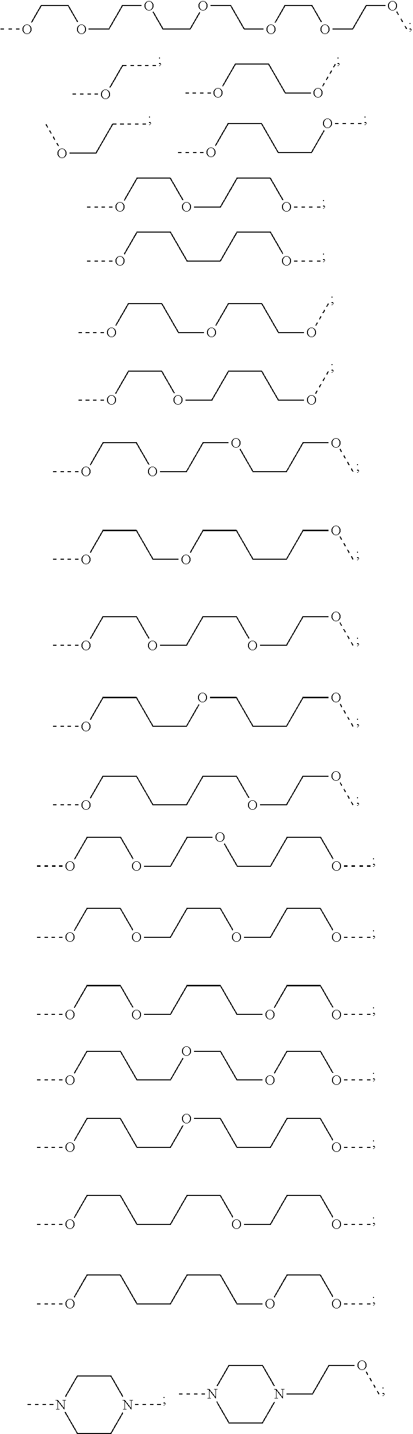

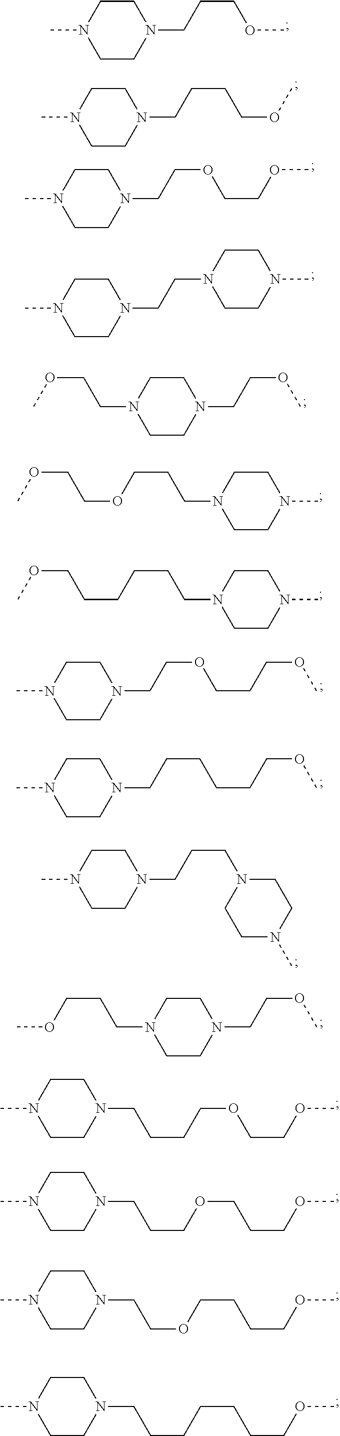

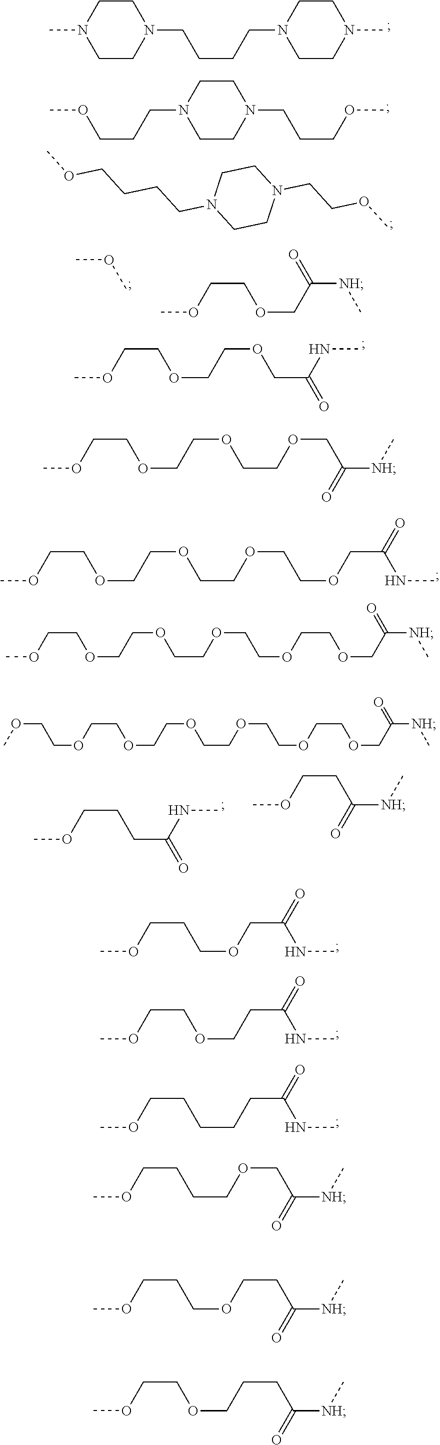

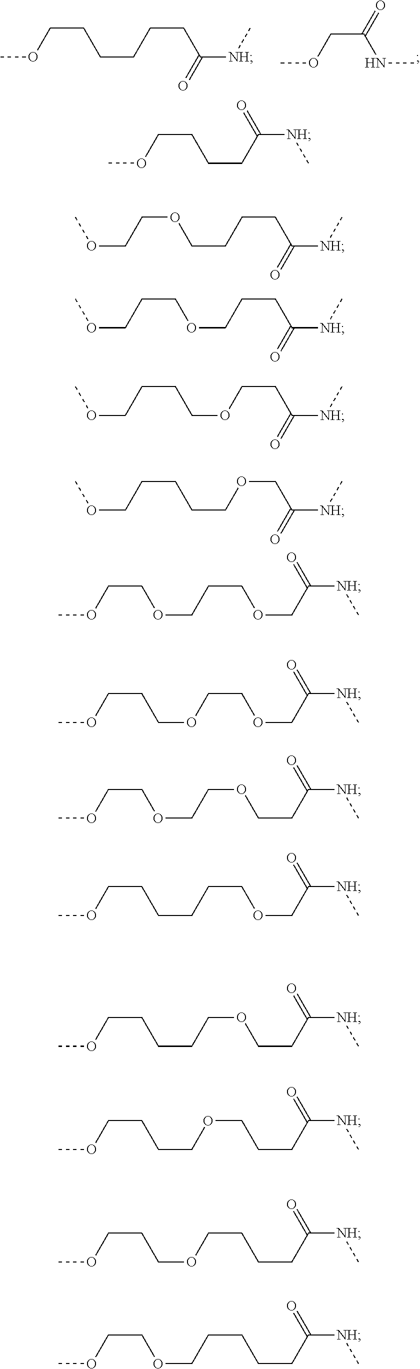

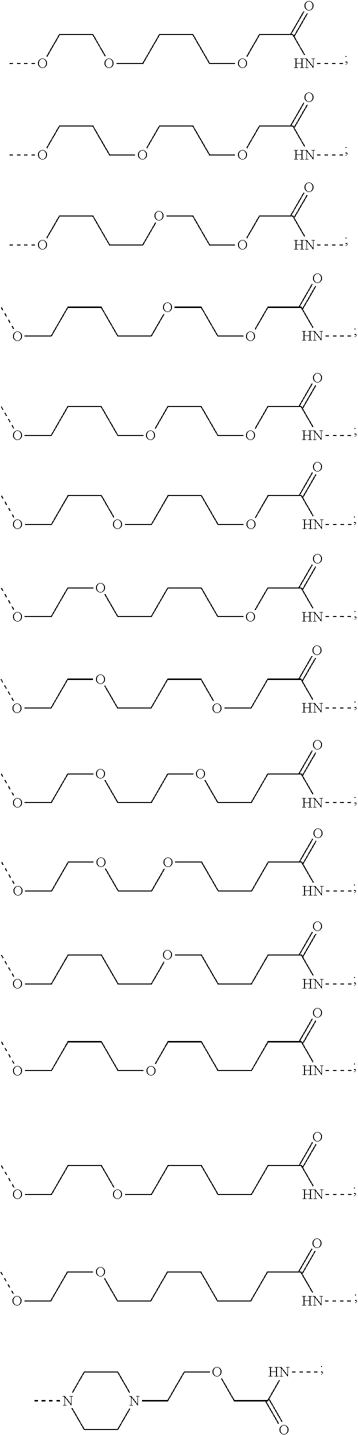

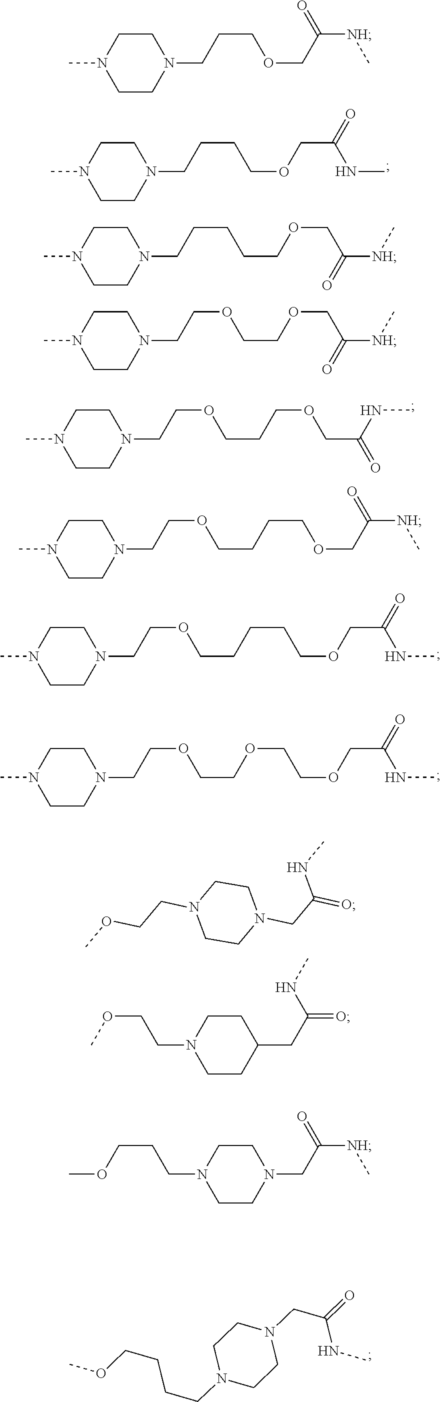

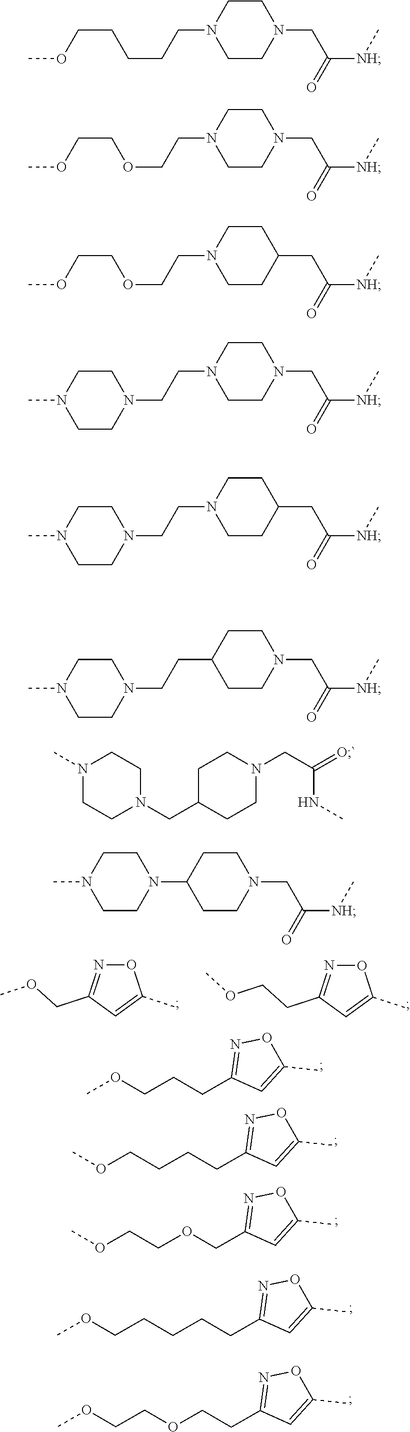

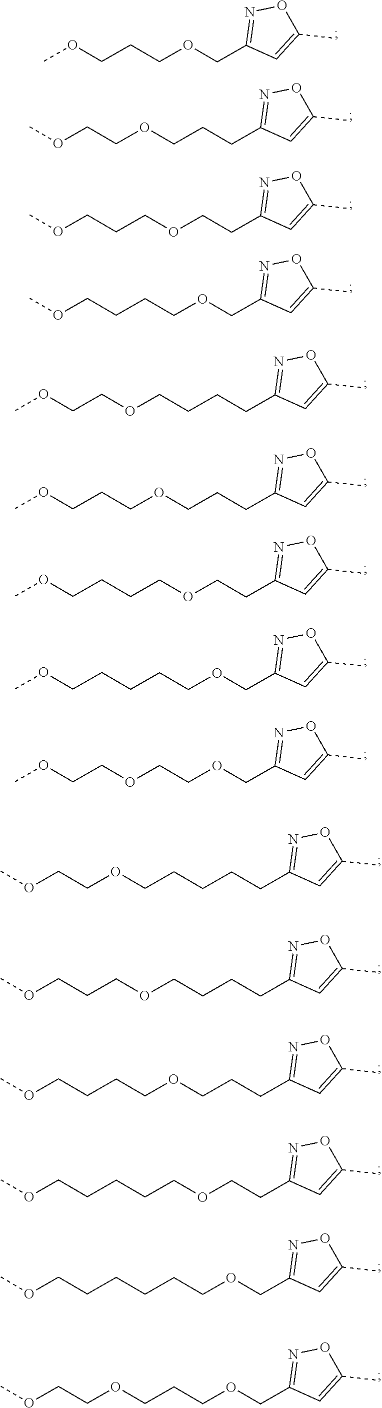

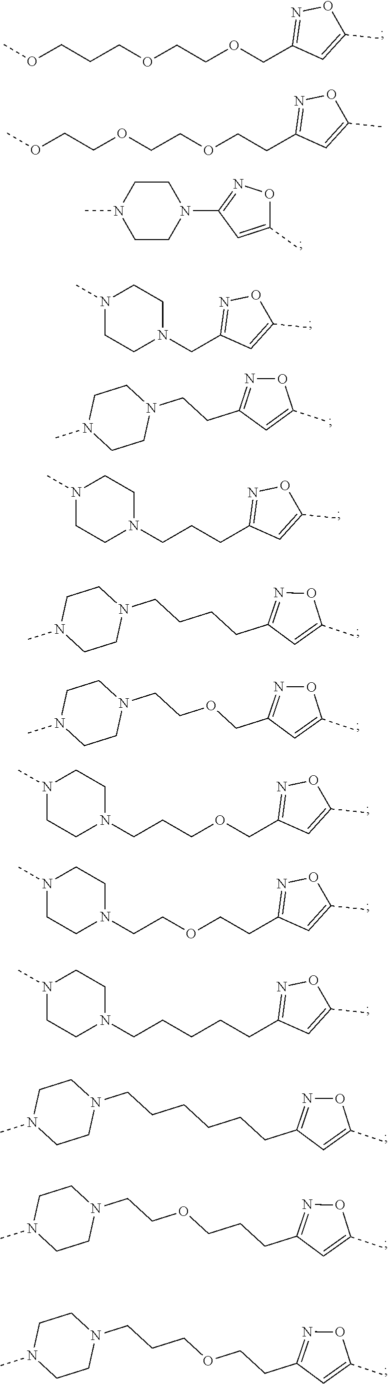

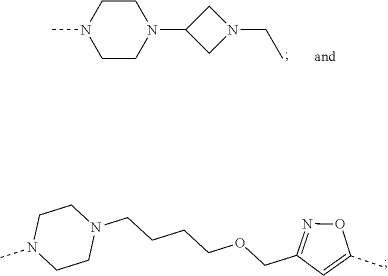

wherein a dashed bond indicates a point of attachment.

In certain embodiments, L has the formula —(C₀₋₁₂ alkylene)- (optionally substituted 3-40 membered heteroalkylene)-(C₀₋₁₂ alkylene)-. In certain embodiments, L is C₄₋₁₄ alkylene. In certain embodiments, L is —(CH₂)₆₋₁₀—.

In certain embodiments, L is —CH₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄—***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)₁₁—***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where *** is a point of attachment to TPL.

In certain embodiments, L is —(C₂₋₂₀ alkylene)-(OCH₂CH₂)₂₋₄—O—(C₀₋₄ alkylene)—***, —(C₂₋₂₀ alkylene)-(OCH₂CH₂)₅₋₇—O—((C₀₋₄ alkylene)—***, —(C₂₋₂₀ alkylene)-(OCH₂CH₂)₈₋₁₀—O—((C₀₋₄ alkylene)—***, —(C₂₋₂₀ alkylene)-(OCH₂CH₂)₁₁₋₁₃—O—((C₀₋₄ alkylene)—***, —(C₂₋₂₀ alkylene)-(OCH₂CH₂)₁₄₋₁₆—O—((C₀₋₄ alkylene)—***, —(C₂₋₂₀ alkylene)-(OCH₂CH₂)₁₇₋₂₀—O—((C₀₋₄ alkylene)—***, —(C₁₋₂₀ alkylene)-(OCH₂CH₂)₁₋₁₀—O—(C₁₋₄ alkylene)-C(O)—***, —(C₁₋₂₀ alkylene)-(OCH₂CH₂)₁₁₋₂₀—O—(C₁₋₄ alkylene)-C(O)—***, —(C₁₋₂₀ alkylene)-(OCH₂CH₂)₁₋₁₀—N(C₁₋₄ alkyl)-C(O)—(C₁₋₄ alkylene)—*** or —(C₁₋₂₀ alkylene)-(OCH₂CH₂)₁₁₋₂₀—N(C₁₋₄ alkyl)-C(O)—(C₁₋₄ alkylene)—***, where *** is a point of attachment to TPL. In certain embodiments, L is —(C₂₋₂₀ alkylene)-(OCH₂CH₂)₂₋₄—O—(C₀₋₄ alkylene)—***, —(C₂₋₂₀ alkylene)-(OCH₂CH₂)₅₋₇—O—((C₀₋₄ alkylene)—***, —(C₂₋₂₀ alkylene)-(OCH₂CH₂)₈₋₁₀—O—((C₀₋₄ alkylene)—***, —(C₂₋₂₀ alkylene)-(OCH₂CH₂)₁₁₋₁₃—O—((C₀₋₄ alkylene)—***, —(C₂₋₂₀ alkylene)-(OCH₂CH₂)₁₄₋₁₆—O—((C₀₋₄ alkylene)—***, or —(C₂₋₂₀ alkylene)-(OCH₂CH₂)₁₇₋₂₀—O—((C₀₋₄ alkylene)—***, where *** is a point of attachment to TPL. In certain embodiments, L is —(C₁₋₂₀ alkylene)-(OCH₂CH₂)₁₋₁₀—O—(C₁₋₄ alkylene)-C(O)—***, —(C₁₋₂₀ alkylene)-(OCH₂CH₂)₁₁₋₂₀—O—(C₁₋₄ alkylene)-C(O)—***, —(C₁₋₂₀ alkylene)-(OCH₂CH₂)₁₋₁₀—N(C₁₋₄ alkyl)-C(O)—(C₁₋₄ alkylene)—***, or —(C₁₋₂₀ alkylene)-(OCH₂CH₂)₁₁₋₂₀—N(C₁₋₄ alkyl)-C(O)—(C₁₋₄ alkylene)—***, where *** is a point of attachment to TPL.

In certain embodiments, L is —(C₂₋₂₀ alkylene)-(OCH₂CH₂)₂₋₄—(C₀₋₄ alkylene)—***, —(C₂₋₂₀ alkylene)-(OCH₂CH₂)₅₋₇—(C₀₋₄ alkylene)—***, —(C₂₋₂₀alkylene)-(OCH₂CH₂)₈₋₁₀—(C₀₋₄ alkylene)—***, —(C₂₋₂₀ alkylene)-(OCH₂CH₂)₁₁₋₁₃—(C₀₋₄ alkylene)—***, —(C₂₋₂₀ alkylene)-(OCH₂CH₂)₁₄₋₁₆—(C₀₋₄ alkylene)—***, —(C₂₋₂₀ alkylene)-(OCH₂CH₂)₁₇₋₂₀—(C₀₋₄ alkylene)—***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to TPL.

In certain embodiments, L is —O(CH₂CH₂O)₂₋₄—(C₀₋₄ alkylene)—***, —O(CH₂CH₂O)₅₋₇—(C₀₋₄ alkylene)—***, —O(CH₂CH₂O)₈₋₁₀—(C₀₋₄ alkylene)—***, —O(CH₂CH₂O)₁₁₋₁₃—(C₀₋₄ alkylene)—***, —O(CH₂CH₂O)₁₄₋₁₆—(C₀₋₄ alkylene)—***, —O(CH₂CH₂O)₁₁₋₂₀—(C₀₋₄ alkylene)—***, —O(CH₂CH₂O)₂₋₁₀—(C₀₋₄ alkylene)C(O)—***, or —O(CH₂CH₂O)₁₁₋₂₀—(C₀₋₄ alkylene)C(O)—*** where *** is a point of attachment to TPL.

In certain embodiments, L is —(C₀₋₂₀ alkylene)-(OCH₂CH₂)₁₋₁₀—(N(C₁₋₄ alkyl))—***, —(C₀₋₂₀ alkylene)-(OCH₂CH₂)₁₁₋₂₀—(N(C₁₋₄ alkyl))—***, —(C₀₋₂₀ alkylene)-(CH₂CH₂O)₁₁₋₂₀—(C₂₋₁₀ alkylene)-(N(C₁₋₄ alkyl))—(C₀₋₁₀ alkylene)—***, or —(C₀₋₂₀ alkylene)-(CH₂CH₂O)₁₁₋₂₀—(C₂₋₁₀ alkylene)-(N(C₁₋₄ alkyl))—(C₀₋₁₀ alkylene)—***, where *** is a point of attachment to TPL.

In certain embodiments, L is —(C₂₋₈ alkylene)-(OCH₂CH₂)₁₋₁₀—(N(C₁₋₄ alkyl))—***, where *** is a point of attachment to TPL.

In certain embodiments, L is one of the following:

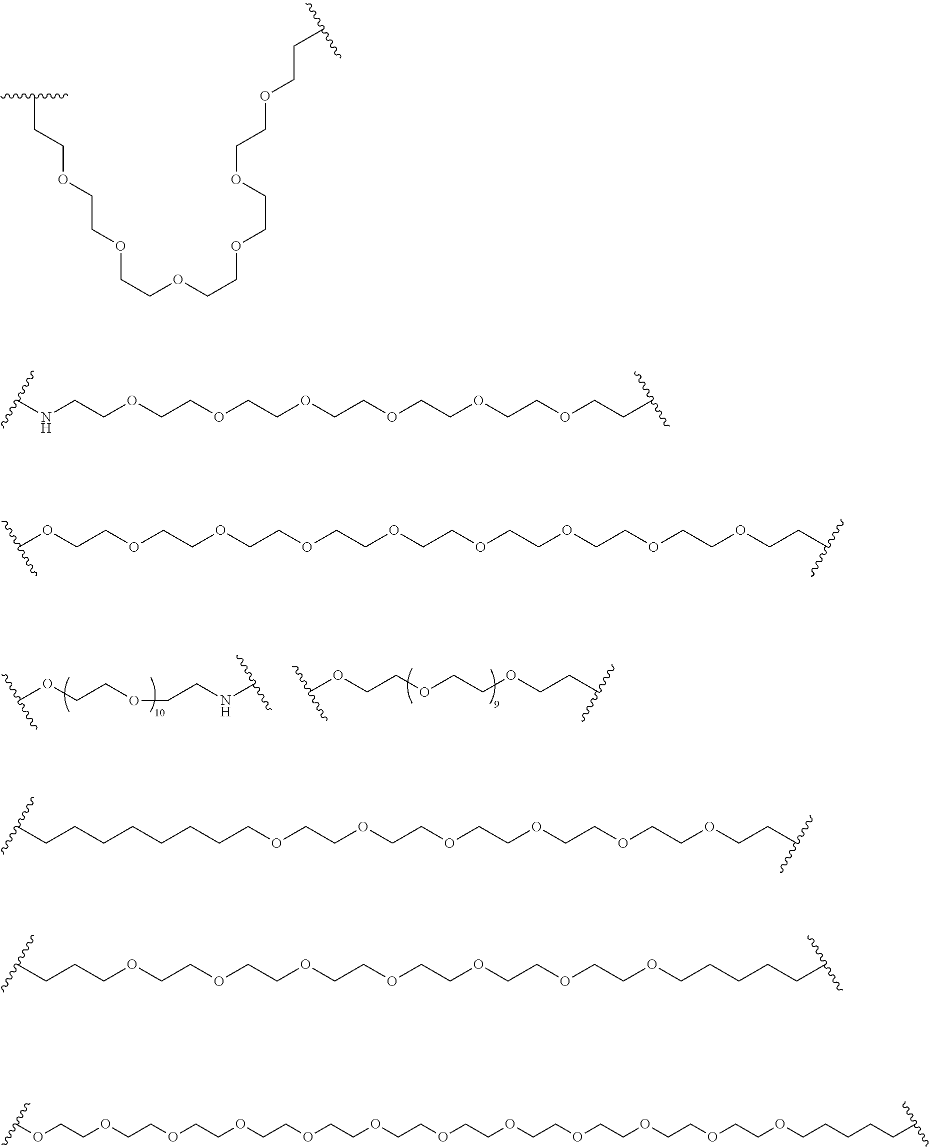

In certain embodiments, L is selected from those depicted in the compounds in Table 3, below. In certain embodiments, L is selected from those depicted in the compounds in Table 3A, below.

### Exemplary Specific Compounds

In certain embodiments, the compound is a compound in Table 2, or a pharmaceutically acceptable salt thereof. In certain embodiments, the compound is a compound in Table 2. In certain embodiments, the compound is a compound in Table 3, or a pharmaceutically acceptable salt thereof. In certain embodiments, the compound is a compound in Table 3. In certain embodiments, the compound is a compound in Table 3A, or a pharmaceutically acceptable salt thereof. In certain embodiments, the compound is a compound in Table 3A. In certain embodiments, the compound is a compound in Table 4, or a pharmaceutically acceptable salt thereof. In certain embodiments, the compound is a compound in Table 4. In certain embodiments, the compound is a compound in Table 5, or a pharmaceutically acceptable salt thereof. In certain embodiments, the compound is a compound in Table 5.

### Synthetic Methods

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing heterobifunctional compounds D. Coupling compound A (a precursor of TPL, for example, a discrete compound that is a target protein ligand) with L′ (a precursor to linker L, containing functionality for coupling to the precursors of both TPL and EPL) affords intermediate B (wherein L″ is a precursor to linker L that contains functionality for coupling to the EPL precursor). Coupling intermediate B with compound C (a precursor of EPL) affords heterobifunctional compound D. Alternatively, the order of coupling compounds A and C to L′ may be reversed, such that L′ is first coupled with compound C, before being coupled to compound A.

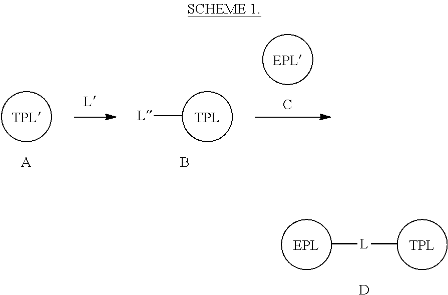

The coupling of compound A with L′, and the coupling of intermediate B with compound C, can be accomplished with a wide variety of strategies. For example, amide coupling conditions can be employed when compound A (or compound C) is to be attached at a modifiable nitrogen atom and L′ (or L″) contains a carboxylic acid group, or vice versa (i.e. compound A contains a carboxylic acid group and L′ contains a nucleophilic amine nitrogen atom). Alternatively, reductive amination conditions can be employed when compound A (or compound C) is to be attached at a modifiable nitrogen atom and L′ (or L″) contains an aldehyde group, or vice versa. Alternatively, nucleophilic substitution conditions can be employed when compound A (or compound C) is to be attached at a modifiable oxygen, nitrogen, or sulfur atom and L′ (or L″) contains a leaving group (such as an alkyl triflate, α-bromoketone, or aryl chloride), or vice versa. As yet another option, transition-metal-mediated coupling conditions can be employed when compound A (or compound C) is to be attached at a modifiable carbon, oxygen, or nitrogen atom (where the carbon atom may be activated, for example, with a bromide or sulfonate) and L′ (or L″) contains a suitable coupling partner (for example, an olefin for a Heck coupling, a trialkylstannane for a Stille coupling, or a boronic acid or boronate ester for a Suzuki coupling, Buchwald-Hartwig amination, or Chan-Lam coupling), or vice versa.

It is understood by one skilled in the art of organic synthesis that protecting group strategies may be employed as necessary, for example, if L′ contains two of the same functional group that are to be selectively coupled to compound A and compound C. For example, L′ may contain, for example, both an unprotected carboxylic acid for coupling to compound A, and a carboxylic acid group that is protected (for example, as a methyl or benzyl ester) during the coupling with compound A and subsequently deprotected (for example, via basic hydrolysis of a methyl ester or hydrogenolysis of a benzyl ester) prior to coupling with compound C.

### II. Therapeutic Applications

The heterobifunctional compounds described herein, such as a compound of Formula I or II, or other compounds in Section I, provide therapeutic benefits to patients suffering from cancer and/or hepatitis. Accordingly, one aspect of the invention provides a method of treating cancer. The method comprises administering to a patient in need thereof a therapeutically effective amount of a compound described herein, such as a compound of Formula I or II, or other compounds in Section I, to treat the cancer. In certain embodiments, the particular compound of Formula I or II is a compound defined by one of the embodiments described above.

Another aspect of the invention provides a method of treating hepatitis. The method comprises administering to a patient in need thereof a therapeutically effective amount of a compound described herein, such as a compound of Formula I or II, or other compounds in Section I, to treat the hepatitis. In certain embodiments, the particular compound of Formula I or II is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitt's Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastases, glioblastoma multiforme, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

### Hepatitis

In certain embodiments, the disease to be treated is hepatitis. In certain embodiments, the hepatitis is hepatitis A, B, or C.

### Causing Death of Cancer Cell

Another aspect of the invention provides a method of causing death of a cancer cell. The method comprises contacting a cancer cell with an effective amount of a compound described herein, such as a compound of Formula I or II, or other compounds in Section I, to cause death of the cancer cell. In certain embodiments, the particular compound of Formula I or II is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.”

### Protein Degradation

Another aspect of the invention provides a method of degrading an effector protein in a cell, wherein the method comprises administering to the cell an effective amount of a compound described herein, such as a compound of Formula II, resulting in degradation of the effector protein in the cell, wherein the effector protein is GSPT1, Cyclin K, RBM23, RBM39, IKZF1, IKZF3, a PLK1 degrader protein, a CDK4 degrader protein, or CK1alpha. In certain embodiments, the effector protein is GSPT1. In certain embodiments, the effector protein is Cyclin K. In certain embodiments, the effector protein is RBM23. In certain embodiments, the effector protein is RBM39. In certain embodiments, the effector protein is IKZF1. In certain embodiments, the effector protein is IKZF3. In certain embodiments, the effector protein is a PLK1 degrader protein. In certain embodiments, the effector protein is a CDK4 degrader protein. In certain embodiments, the effector protein is CK1alpha.

In certain embodiments, the cell is a cell that expresses a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα. In certain embodiments, the cell is a cancer cell that expresses a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα. In certain embodiments, the target protein is KRAS. In certain embodiments, the target protein is HER2. In certain embodiments, the target protein is BTK. In certain embodiments, the target protein is EGFR. In certain embodiments, the target protein is androgen receptor protein. In certain embodiments, the target protein is estrogen receptor protein. In certain embodiments, the target protein is ALK. In certain embodiments, the target protein is IDH1. In certain embodiments, the target protein is FLT3. In certain embodiments, the target protein is FGFR1. In certain embodiments, the target protein is FGFR4. In certain embodiments, the target protein is HCV-NS3. In certain embodiments, the target protein is FGFR2. In certain embodiments, the target protein is FGFR3. In certain embodiments, the target protein is ERK1. In certain embodiments, the target protein is ERK2. In certain embodiments, the target protein is FGR. In certain embodiments, the target protein is HER3. In certain embodiments, the target protein is HER4. In certain embodiments, the target protein is PI3Kα. In certain embodiments, the cell is a cancer cell, wherein the cancer is one of those described above in the section entitled “Cancer.”

Another aspect of the invention provides a method of degrading a GSPT1 protein in a cell, wherein the method comprises administering to the cell an effective amount of a compound described herein, such as a compound of Formula I, resulting in degradation of the GSPT1 protein in the cell. In certain embodiments, the cell is a cell that expresses a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα. In certain embodiments, the cell is a cancer cell that expresses a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα. In certain embodiments, the target protein is KRAS. In certain embodiments, the target protein is HER2. In certain embodiments, the target protein is BTK. In certain embodiments, the target protein is EGFR. In certain embodiments, the target protein is androgen receptor protein. In certain embodiments, the target protein is estrogen receptor protein. In certain embodiments, the target protein is ALK. In certain embodiments, the target protein is IDH1. In certain embodiments, the target protein is FLT3. In certain embodiments, the target protein is FGFR1. In certain embodiments, the target protein is FGFR4. In certain embodiments, the target protein is HCV-NS3. In certain embodiments, the target protein is FGFR2. In certain embodiments, the target protein is FGFR3. In certain embodiments, the target protein is ERK1. In certain embodiments, the target protein is ERK2. In certain embodiments, the target protein is FGR. In certain embodiments, the target protein is HER3. In certain embodiments, the target protein is HER4. In certain embodiments, the target protein is PI3Kα. In certain embodiments, the cell is a cancer cell, wherein the cancer is one of those described above in the section entitled “Cancer.”

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011450), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I, Formula II, or other compounds in Section I) in the manufacture of a medicament. In certain embodiments, the medicament is for treating a disease described herein, such as cancer.

Another aspect of the invention provides for the use of a compound described herein (such as a compound of Formula I, Formula II, or other compounds in Section I) for treating a medical disease, such a disease described herein (e.g., cancer).

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I or II) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. Medical Kits

Another aspect of this invention is a kit comprising (i) a compound described herein, such as a compound of Formula I, and (ii) instructions for use, such as treating cancer.

### EXAMPLES

The invention now being generally described, will be more readily understood by reference to the following examples, which are included merely for purposes of illustration of certain aspects and embodiments of the present invention, and are not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), Dichloromethane (CH₂Cl₂), and dimethylformamide (DMF) was dried by a PureSolv™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 100 MHz ¹³C) spectrometer at room temperature. Chemical shifts were reported in ppm relative to the residual CDCl₃ (δ 7.26 ppm ¹H; δ 77.0 ppm ¹³C), CD₃OD (δ 3.31 ppm ¹H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=1.0 mL/min, 20 min), or as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography.

TABLE 1
LC-MS Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |
| | Post time(min) | 0 | | |
| | Column Temp | 50° C. | | |
| | Detector | DAD | | |
| MS | Ionization source | ESI | | |
| | Drying Gas | N₂ | | |
| | Drying Gas Flow | 10 (L/min) | | |
| | Nebulizer Pressure | 40 (psi) | | |
| | Drying Gas | 350° C. | | |
| | Temperature | | | |

| | |
|---|---|
| Capillary Voltage | 2500 (V) |
| MS Polarity | Positive |
| MS Mode | Scan |
| Mass Range | 100-1500 |

LC-MS Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |
| | Post time(min) | 0 | | |
| | Column Temp | 50° C. | | |
| | Detector | DAD | | |
| MS | Ionization source | ESI | | |
| | Drying Gas | N₂ | | |
| | Drying Gas Flow | 10 (L/min) | | |
| | Nebulizer Pressure | 40 (psi) | | |
| | Drying Gas | 350° C. | | |
| | Temperature | | | |

| | |
|---|---|
| Capillary Voltage | 2500 (V) |
| MS Polarity | Positive |
| MS Mode | Scan |
| Mass Range | 100-1500 |

LC-MS Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |
| | Post time(min) | 0 | | |
| | Column Temp | 50° C. | | |
| | Detector | DAD | | |
| MS | Ionization source | ESI | | |
| | Drying Gas | N₂ | | |
| | Drying Gas Flow | 10 (L/min) | | |
| | Nebulizer Pressure | 40 (psi) | | |
| | Drying Gas | 350° C. | | |
| | Temperature | | | |

| | |
|---|---|
| Capillary Voltage | 2500 (V) Positive |
| MS Polarity | Positive |
| MS Mode | Scan |
| Mass Range | 100-1500 |

LC-MS METHOD 40

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 40 | 0.8 |
| | | 0.40 | 40 | 0.8 |
| | | 3.40 | 100 | 0.8 |
| | | 3.90 | 100 | 0.8 |
| | | 3.91 | 40 | 0.8 |
| | | 4.00 | 40 | 1.0 |
| | | 4.50 | 40 | 1.0 |
| | Post time(min) | 0 | | |
| | Column Temp | 50° C. | | |
| | Detector | DAD (Agilent 1100)/ELSD(Agilent 1260 Infinity) | | |
| MS | Ionization source | ESI | | |
| | Drying Gas | N₂ | | |
| | Drying Gas Flow | 10 (L/min) | | |
| | Nebulizer Pressure | 2070 (Torr) | | |
| | Drying Gas | 350° C. | | |
| | Temperature | | | |

| | |
|---|---|
| Capillary Voltage | 2500(V) Positive |

LC-MS Method 595

| Instrument | | SHIMADZU LCMS-2020; | | |
|---|---|---|---|---|
| Software | | LabSolution Version 5.93 | | |
| HPLC | Column | Kinetex EVO C18 2.1 × 30 mm, 5 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.0 | 5 | 1.5 |
| | | 0.80 | 95 | 1.5 |
| | | 1.20 | 95 | 1.5 |
| | | 1.21 | 5 | 1.5 |
| | | 1.55 | 5 | 1.5 |

| | | |
|---|---|---|
| | Column Temp | 50° C. |
| | Detector | PDA (220 nm & 254 nm) |
| MS | Ionization source | ESI |
| | Drying Gas Flow | 15 (L/min) |
| | DL Voltage | 120 (v) |
| | Qarray DC Voltage | 20 (V) |
| | MS Polarity | Positive |
| | MS Mode | Scan |
| | Mass range | 100-1000 |

LC-MS Method 595CD

| Instrument | | SHIMADZU LCMS-2020 | | |
|---|---|---|---|---|
| Software | | LabSolutions Version 5.93 | | |
| HPLC | Column | Kinetex EVO C18 2.1 × 30 mm, 5 μm | | |
| | Mobile Phase | A: 0.025% NH₃•H₂O in water (v/v) | | |
| | | B: Acetonitrile | | |
| | Gradient | Time (min) | B (%) | Flow (mL/min) |
| | | 0.00 | 5 | 1.5 |
| | | 0.80 | 95 | 1.5 |
| | | 1.2 | 95 | 1.5 |
| | | 1.21 | 5 | 1.5 |
| | | 1.55 | 5 | 1.5 |

| | | |
|---|---|---|
| | Column Temp | 40° C. |
| | Detector | PDA (220 & 254 nm) |
| MS | Ionization source | ESI |
| | Drying Gas | N₂ |
| | Drying Gas Flow | 15 (L/min) |
| | DL Voltage | 120 (V) |
| | Qarray DC Voltage | 20 (V) |
| | MS Polarity | Positive |
| | MS Mode | Scan |
| | Mass range | 100-1000 |

LC-MS Method 060

| Instrument | | Agilent 1200\G6110A | | |
|---|---|---|---|---|
| Software | | Agilent ChemStation Rev. B. 04.03[54] | | |
| HPLC | Column | Kinetex@ 5 μm EVO C18 30*2.1 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.01 | 0 | 1.5 |
| | | 0.80 | 60 | 1.5 |
| | | 1.20 | 60 | 1.5 |
| | | 1.21 | 0 | 1.5 |
| | | 1.5 | 0 | 1.5 |

| | | |
|---|---|---|
| | Column Temp | 50° C. |
| | Detector | DAD (220 & 254 nm) |
| MS | Ionization source | ESI |
| | Drying Gas | N₂ |
| | Drying Gas Flow | 11 (L/min) |
| | Nebulizer Pressure | 60 (psig) |
| | Drying Gas Temp | 350 (° C.) |
| | Capillary Voltage | 3500 (V) |
| | MS Polarity | Positive |
| | MS Mode | Scan |
| | Mass range | 100-1000 |

### Example 1—Preparation of Compound I-1

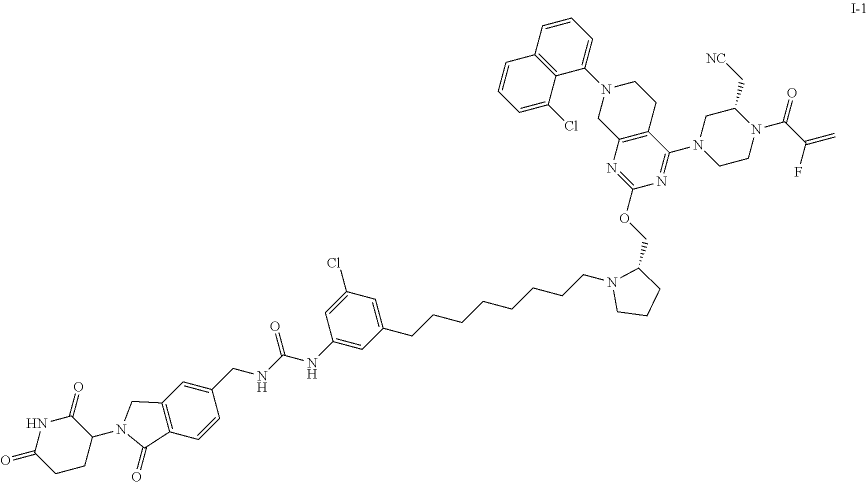 [SMILES: C=C(F)C(=O)N1CCN(c2nc(OC[C@@H]3CCCN3CCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N]

The title compound was prepared according to the following procedures.

### 1. Preparation of Compound 2

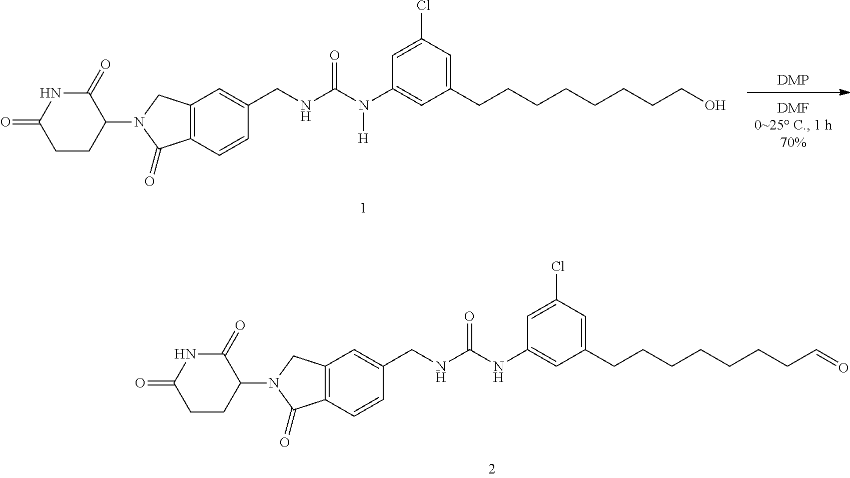

To a solution of 1-[3-chloro-5-(8-hydroxyoctyl)phenyl]-3-[[2-(2,6-dioxo-3-piperidyl)-1-oxo-isoindolin-5-yl]methyl]urea (50.0 mg, 90.1 μmol, 1.0 equiv) in DMF (2.5 mL) was added DMP (57.3 mg, 135 μmol, 1.5 equiv) at 0° C. The mixture was warmed to 25° C. and stirred for 1 h. The reaction mixture was diluted with saturated aqueous NaHCO₃ (8 mL) and extracted with EtOAc (4 mL, X3). The combined organic phase was washed with brine (5 mL), dried over anhydrous Na₂SO₄, filtered and concentrated in vacuo. The residue was purified by preparative TLC on silica gel (Dichloromethane/Methanol=10/1) to afford 1-[3-chloro-5-(8-oxooctyl)phenyl]-3-[[2-(2,6-dioxo-3-piperidyl)-1-oxo-isoindolin-5-yl]-methyl]urea (35.0 mg, 63.0 μmol, 70% yield, 99% purity) as a yellow solid. ¹H NMR (400 MHz, CDCl₃) δ 9.72-9.64 (m, 1H), 8.53-8.33 (m, 1H), 8.00-7.94 (m, 1H), 7.48-7.25 (m, 3H), 7.14-7.06 (m, 1H), 6.83-6.66 (m, 1H), 5.21-4.92 (m, 1H), 4.47-4.09 (m, 4H), 2.46-2.29 (m, 4H), 2.18-2.04 (m, 1H), 1.87-1.40 (m, 9H), 1.33-1.22 (m, 4H). LC-MS: MS (ES⁺). RT=0.938 min, m/z=553.2 [M+H+].

### 2. Preparation of Compound I-1

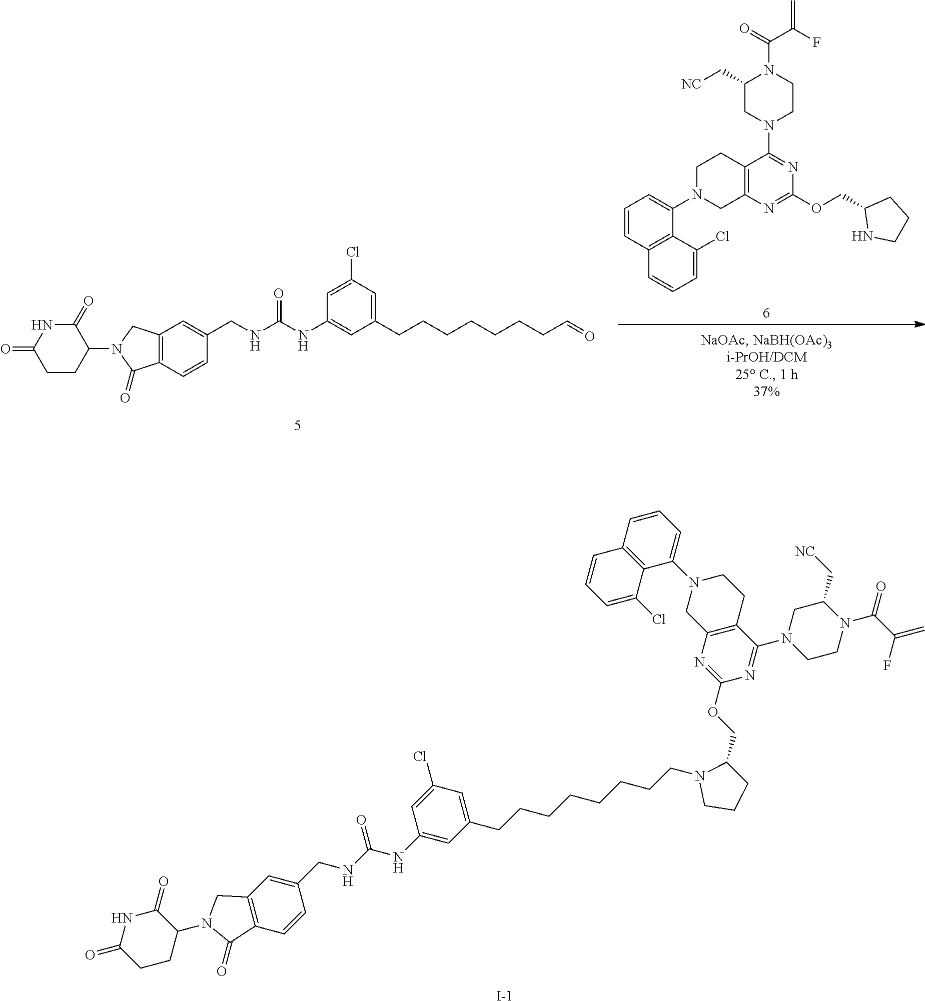 [SMILES: O=CCCCCCCCc1cc(Cl)cc(NC(=O)NCc2ccc3c(c2)CN(C2CCC(=O)NC2=O)C3=O)c1 ; C=C(F)C(=O)N1CCN(c2nc(OC[C@@H]3CCCN3)nc3c2CCN(c2cccc4cccc(Cl)c24)C3)C[C@@H]1CC#N ; "6" ; C=C(F)C(=O)N1CCN(c2nc(OC[C@@H]3CCCN3CCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N ; [I-]]

A mixture of 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (25.0 mg, 35.5 μmol, 1.0 equiv, TFA salt) and NaOAc (14.6 mg, 177 μmol, 5.0 equiv) in i-PrOH (1 mL) and DCM (1 mL) was stirred at 25° C. for 15 min. To the mixture were added 1-[3-chloro-5-(8-oxooctyl)phenyl]-3-[[2-(2,6-dioxo-3-piperidyl)-1-oxo-isoindolin-5-yl]methyl]urea (29.5 mg, 53.3 μmol, 1.5 equiv) and NaBH(OAc)₃ (37.6 mg, 177 μmol, 5.0 equiv). The mixture was stirred at 25° C. for 45 min. The reaction mixture was diluted with water (8 mL) and extracted with DCM (5 mL*3). The combined organic phase was dried over anhydrous Na₂SO₄, filtered and concentrated in vacuo. The residue was purified by preparative TLC on silica gel (Dichloromethane/Methanol=10/1) and then prep-HPLC (column: Phenomenex Gemini-NX C18 75*30 mm*3 um; mobile phase: [water (0.05% HCl)-ACN]; B %: 30%-60%, 7 minutes) to afford 1-[3-chloro-5-[8-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]octyl]phenyl]-3-[[2-(2,6-dioxo-3-piperidyl)-1-oxo-isoindolin-5-yl]methyl]urea (15.0 mg, 13.0 μmol, 37% yield, 99% purity) as a white solid. ¹H NMR (400 MHz, DMSO-d₆) δ 10.99 (s, 1H), 8.86 (s, 1H), 7.94-7.88 (m, 1H), 7.78-7.66 (m, 2H), 7.62-7.48 (m, 3H), 7.47-7.39 (m, 3H), 7.38-7.28 (m, 1H), 7.10-7.04 (m, 1H), 6.92-6.83 (m, 1H), 6.78-6.70 (m, 1H), 5.46-5.21 (m, 2H), 5.14-5.06 (m, 1H), 4.50-3.83 (m, 13H), 3.81-3.67 (m, 2H), 3.55-3.47 (m, 2H), 3.16-3.05 (m, 4H), 2.99-2.87 (m, 3H), 2.65-2.57 (m, 3H), 2.45-2.35 (m, 3H), 2.04-1.92 (m, 2H), 1.86-1.63 (m, 3H), 1.55-1.41 (m, 4H), 1.29-1.12 (m, 9H). LC-MS: MS (ES⁺): RT=2.761 min, m/z=1126.4 [M+H⁺].

### Example 2—Preparation of Compound I-2

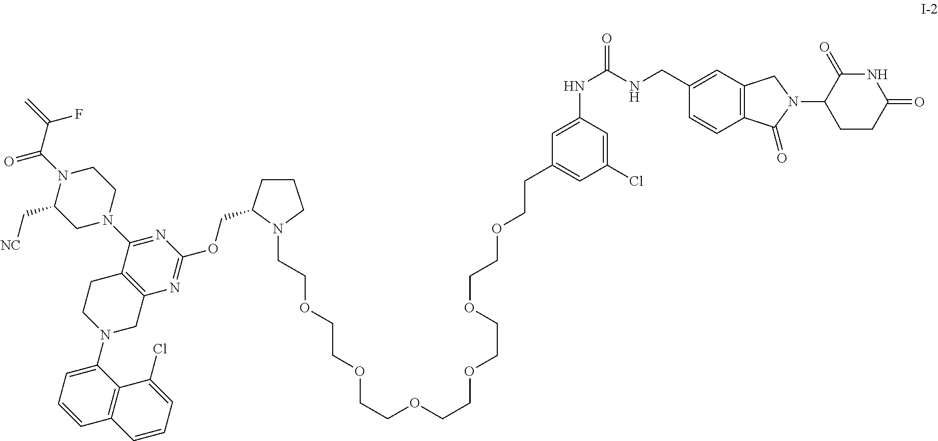 [SMILES: C=C(F)C(=O)N1CCN(c2nc(OC[C@@H]3CCCN3CCOCCOCCOCCOCCOCCOCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N]

The title compound was prepared according to the following procedures.

### 1. Preparation of Compound 2

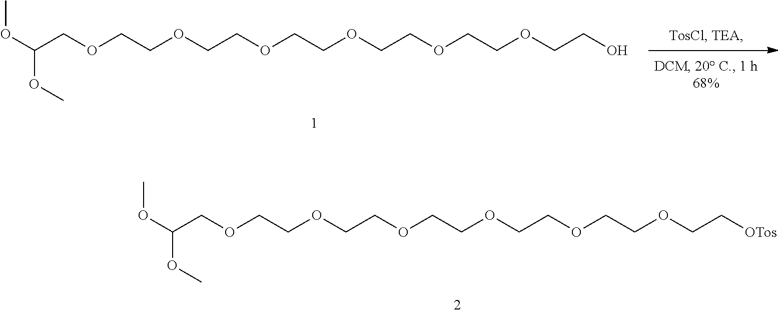

To a solution of compound 1 (4.0 g, 10.8 mmol, 1.0 equiv) in DCM (40 mL) was added Et₃N (2.19 g, 21.60 mmol, 3.0 mL, 2.0 equiv) and TosCl (3.1 g, 16.2 mmol, 1.5 equiv). The mixture was stirred at 20° C. for 1 h. The reaction mixture was concentrated under reduced pressure to give a residue. The residue was purified by column chromatography (SiO₂, Petroleum ether/Ethyl acetate=2/1 to 2/3). Compound 2 (3.9 g, 7.43 mmol, 68% yield) was obtained as a yellow oil. ¹HNMR (400 MHz, CDCl3): δ 7.81 (d, J=8.2 Hz, 2H), 7.35 (d, J=7.9 Hz, 2H), 4.52 (t, J=5.1 Hz, 1H), 4.17 (t, J=4.8 Hz, 2H), 3.72-3.60 (m, 20H), 3.59 (m, 4H), 3.55 (d, J=5.1 Hz, 2H), 3.40 (s, 6H), 2.46 (s, 3H).

### 2. Preparation of Compound 3

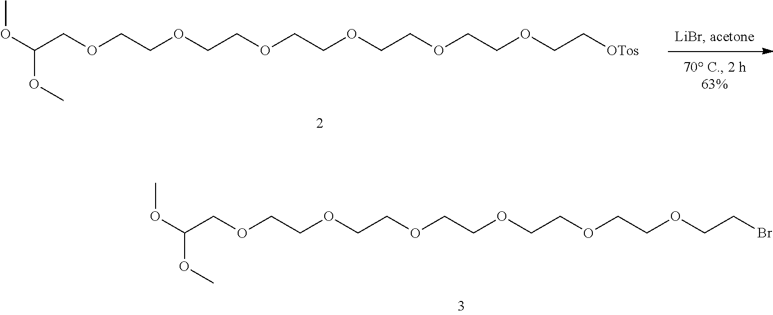

A mixture of compound 2 (2.47 g, 4.71 mmol, 1.0 equiv) and LiBr (2.05 g, 23.55 mmol, 591 μL, 5.0 equiv) in acetone (30 mL) was stirred at 70° C. for 2 h. The reaction mixture was partitioned between EtOAc (50 mL) and H₂O (50 mL). The organic phase was separated, washed with brine (50 mL), dried over Na₂SO₄, filtered and concentrated under reduced pressure to give the crude compound 3 (1.3 g, 3.0 mmol, 63% yield) as a yellow oil. ¹HNMR (400 MHz, CDCl₃): δ 4.52 (t, J=5.2 Hz, 1H), 4.61-4.45 (m, 1H), 3.82 (t, J=6.3 Hz, 2H), 3.69-3.65 (m, 20H), 3.55 (d, J=5.3 Hz, 2H), 3.48 (t, J=6.3 Hz, 2H), 3.40 (s, 6H).

### 3. Preparation of Compound 5

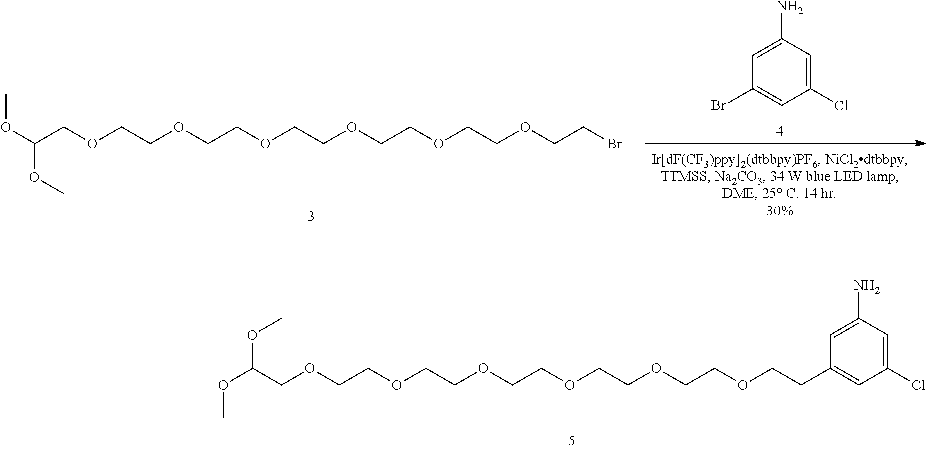

To an 40 mL vial equipped with a stir bar was added compound 3 (1.36 g, 3.15 mmol, 1.3 equiv), compound 4 (500 mg, 2.42 mmol, 1.0 equiv), Ir[dF(CF₃)ppy]2(dtbpy)(PF₆) (27 mg, 24 μmol, 0.01 equiv), NiCl₂·dtbbpy (5 mg, 12 μmol, 0.005 equiv), TTMSS (602 mg, 2.42 mmol, 747 μL, 1.0 equiv), Na₂CO₃ (513 mg, 4.84 mmol, 2.0 equiv) in DME (20 mL). The vial was sealed and placed under nitrogen was added. The reaction was stirred and irradiated with a 34 W blue LED lamp (7 cm away), with cooling fan to keep the reaction temperature at 25° C. for 14 h. The reaction mixture was filtered and concentrated under reduced pressure to give a residue. The residue was purified by column chromatography (SiO₂, Petroleum ether/Ethyl acetate=1/1 to EA/MeOH=10/1). The residue was purified by prep-HPLC (column: Waters Xbridge C18 150*50 mm*10 um; mobile phase: [water (10 mM NH₄HCO₃)-ACN]; B %: 26%-56%, min). Compound 5 (0.36 g, 750 μmol, 30% yield) was obtained as a yellow oil. LC-MS: MS (ES⁺): m/z=480.2 [M+H⁺].

### 4. Preparation of Compound 7

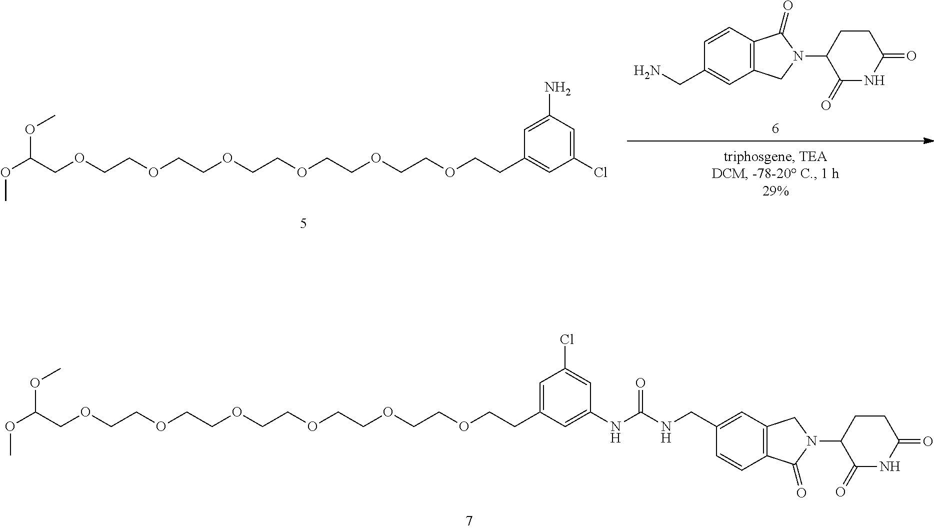 [SMILES: COC(COCCOCCOCCOCCOCCOCCc1cc(N)cc(Cl)c1)OC ; NCc1ccc2c(c1)CN(C1CCC(=O)NC1=O)C2=O ; "6" ; COC(COCCOCCOCCOCCOCCOCCc1cc(Cl)cc(NC(=O)NCc2ccc3c(c2)CN(C2CCC(=O)NC2=O)C3=O)c1)OC]

To a solution of bis(trichloromethyl) carbonate (204 mg, 687 μmol, 1.0 equiv) in DCM (30 mL) was added Et₃N (727 mg, 7.18 mmol, 1.0 mL, 10.4 equiv) and compound 5 (330 mg, 687 μmol, 1.0 equiv) in DCM (30 mL) at −78° C., and then it was stirred for 0.5 h. Compound 6 (255 mg, 823 μmol, 1.2 equiv, HCl salt) was added at −78° C. The mixture was added and stirred at 20° C. for 0.5 h. The reaction mixture was poured into aq. NaHCO₃ (50 mL) at 0° C., and then extracted with DCM (50 mL). The combined organic layers were washed with brine (50 mL), dried over Na₂SO₄, filtered and concentrated under reduced pressure to give a residue. The residue was purified by column chromatography (SiO₂, DCM/MeOH=30/1 to 10/1). Compound 7 (160 mg, 205 μmol, 29% yield) was obtained as a yellow solid. ¹HNMR (400 MHz, CDCl₃): δ 8.35 (s, 1H), 8.14 (s, 1H), 7.81-7.56 (m, 2H), 7.49-7.40 (m, 1H), 7.39-7.32 (m, 1H), 7.09-7.03 (m, 1H), 6.79 (s, 1H), 6.68-5.93 (m, 1H), 5.15 (m, 1H), 4.59-4.41 (m, 3H), 4.40-4.21 (m, 2H), 3.58-3.56 (m, 24H), 3.36 (s, 6H), 2.89-2.65 (m, 4H), 2.44-2.27 (m, 1H), 2.25-2.13 (m, 1H).

### 5. Preparation of Compound 8

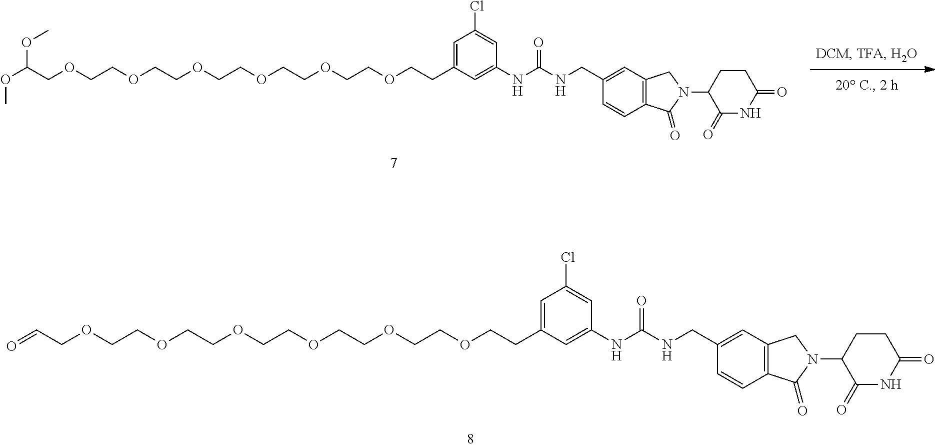

A mixture of compound 7 (60 mg, 76 μmol, 1.0 equiv) in CH₂Cl₂ (2 mL), TFA (0.2 mL) and H₂O (0.04 mL) was stirred at 20° C. for 2 h. The reaction mixture was adjusted with Et₃N to pH=7˜8 at 0° C. The solution of compound 8 was used into the next step without further purification. LC-MS: MS (ES⁺): m/z=733.2 [M+H⁺].

### 6. Preparation of Compound I-2

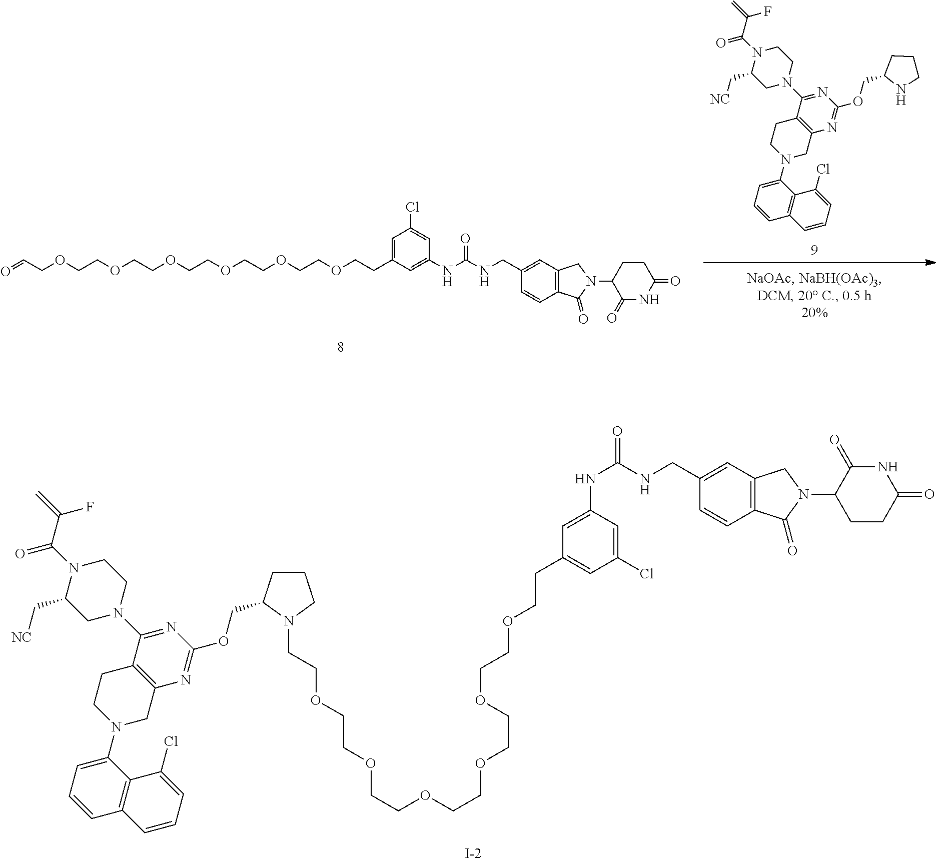

To a solution of compound 8 (56 mg, 76 μmol, 1.0 equiv) in DCM (1 mL) was added Et₃N (22 mg, 223 μmol, 31 μL, 2.9 equiv), compound 9 (50 mg, 71 μmol, 0.90 equiv, TFA salt) and NaBH(OAc)₃ (161 mg, 759 μmol, 9.9 equiv). The mixture was stirred at 20° C. for 0.5 h. The reaction mixture was partitioned between H₂O (20 mL) and DCM (20 mL). The organic phase was separated, dried over Na₂SO₄, filtered and concentrated under reduced pressure to give a residue The residue was purified by prep-HPLC (column: Waters Xbridge 150*25 mm*5 μm; mobile phase: [water (10 mM NH₄HCO₃)-ACN]; B %: 50%-80%, 9 min) to give desired compound 1-(3-chloro-5-(20-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)-3,6,9,12,15,18-hexaoxaicosyl)phenyl)-3-((2-(2,6-dioxopiperidin-3-yl)-1-oxoisoindolin-5-yl)methyl)urea (20 mg, 15 μmol, 20% yield, 97% purity) as a white solid. ¹HNMR (400 MHz, CD₃OD): δ 7.82-7.65 (m, 2H), 7.55-7.42 (m, 6H), 7.40-7.26 (m, 2H), 7.11 (s, 1H), 6.86 (s, 1H), 5.41-5.23 (m, 2H), 5.13 (m, 1H), 4.50 (s, 3H), 4.47-4.42 (m, 2H), 4.42-4.07 (m, 7H), 3.56 (m, 33H), 3.26-3.02 (m, 6H), 2.70 (s, 7H), 2.53-2.31 (m, 1H), 2.15-2.00 (m, 1H), 1.91-1.61 (m, 3H). LC-MS: MS (ES⁺): RT=2.328 min, m/z=653.8 [M/2+H⁺]; LCMS method: LC-MS METHOD 25.

### Example 3—Preparation of Compound I-3

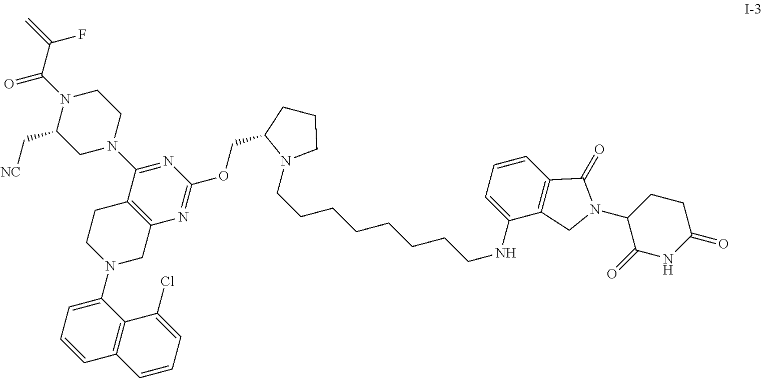 [SMILES: C=C(F)C(=O)N1CCN(c2nc(OC[C@@H]3CCCN3CCCCCCCCNc3cccc4c3CN(C3CCC(=O)NC3=O)C4=O)nc3c2CCN(c2cccc4cccc(Cl)c24)C3)C[C@@H]1CC#N]

The title compound was prepared according to the following procedures.

### 1. Preparation of Compound 3

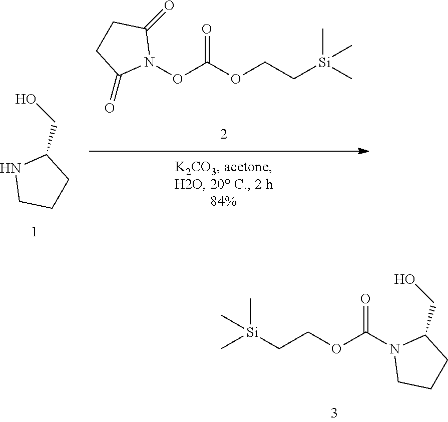 [SMILES: OC[C@@H]1CCCN1 ; C[Si](C)(C)CCOC(=O)ON1C(=O)CCC1=O ; C[Si](C)(C)CCOC(=O)N1CCC[C@H]1CO]

To a solution of compound 1 (1.0 g, 9.89 mmol, 961 μL, 1.0 equiv) in acetone (20 mL) and H₂O (6 mL) was added compound 2 (2.56 g, 9.89 mmol, 1.0 equiv) and K₂CO₃ (4.10 g, 29.66 mmol, 2.7 mL, 3.0 equiv). The mixture was stirred at 20° C. for 2 h. The reaction mixture was concentrated under reduced pressure to give a residue. The reaction mixture was partitioned between H₂O (30 mL) and EtOAc (50 mL). The organic phase was separated, washed with brine (30 mL), dried over Na₂SO₄, filtered and concentrated under reduced pressure to give a residue. The residue was purified by column chromatography (SiO₂, Petroleum ether/Ethyl acetate=1/1). Compound 3 (2.05 g, 8.35 mmol, 84% yield) was obtained as a colorless oil. ¹HNMR (400 MHz, CDCl₃): δ 4.27-4.14 (m, 2H), 4.08-3.92 (m, 1H), 3.72-3.58 (m, 2H), 3.52 (m, 1H), 3.35 (m, 1H), 2.09-2.01 (m, 2H), 1.88-1.75 (m, 2H), 1.69-1.54 (m, 1H), 1.10-0.96 (m, 2H), 0.06 (s, 9H).

### 2. Preparation of Compound 4

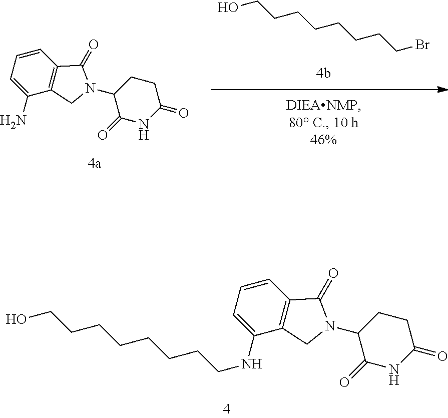 [SMILES: Nc1cccc2c1CN(C1CCC(=O)NC1=O)C2=O ; OCCCCCCCCBr ; O=C1CCC(N2Cc3c(NCCCCCCCCO)cccc3C2=O)C(=O)N1]

To a solution of compound 4a (1.0 g, 3.86 mmol, 1.0 equiv) and compound 4b (806 mg, 3.86 mmol, 661 μL, 1.0 equiv) in NMP (10 mL) was added DIEA (747 mg, 5.79 mmol, 1.0 mL, 1.5 equiv). The mixture was stirred at 80° C. for 10 h. The reaction mixture was diluted with H₂O (50 mL) and the mixture was filtered. The filter cake was concentrated under reduced pressure. The residue was purified by prep-HPLC (column: phenomenex luna C18 150*40 mm*15 μm; mobile phase: [water (0.1% TFA)-ACN]; B %: 23%-53%, 11 min). Compound 4 (700 mg, 1.81 mmol, 46% yield) was obtained as a yellow solid. ¹HNMR (400 MHz, DMSO): δ 11.01 (s, 1H), 7.31-7.24 (m, 1H), 6.92 (d, J=7.5 Hz, 1H), 6.73 (d, J=8.1 Hz, 1H), 5.55 (s, 1H), 5.18-5.04 (m, 1H), 4.43-4.05 (m, 3H), 3.39 (m, 1H), 3.10 (m, 2H), 2.99-2.85 (m, 1H), 2.69-2.56 (m, 1H), 2.37-2.22 (m, 1H), 2.08-1.97 (m, 1H), 1.63-1.51 (m, 2H), 1.45-1.21 (m, 11H). LC-MS: MS (ES⁺): m/z=388.3 [M+H⁺].

### 3. Preparation of Compound 5

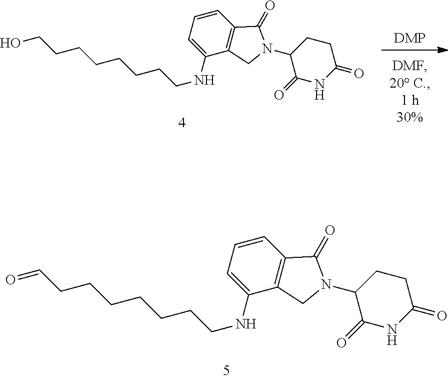

To a solution of compound 4 (0.2 g, 516 μmol, 1.0 equiv) in DMF (2 mL) was added DMP (328 mg, 773 μmol, 239 μL, 1.5 equiv). The mixture was stirred at 20° C. for 1 h. The mixture was added CH₂Cl₂ (80 mL). The mixture was washed with Na₂SO₃ (2 mL) and NaHCO₃ (2 mL), H₂O (2 mL), dried over Na₂SO₄, filtered and concentrated under reduced pressure to give a residue. The residue was purified by prep-TLC (SiO₂, DCM:MeOH=10:1). Compound 5 (60 mg, 155 μmol, 30% yield) was obtained as a white solid. LC-MS: MS (ES⁺): m/z=386.1 [M+H⁺].

### 4. Preparation of Compound 7

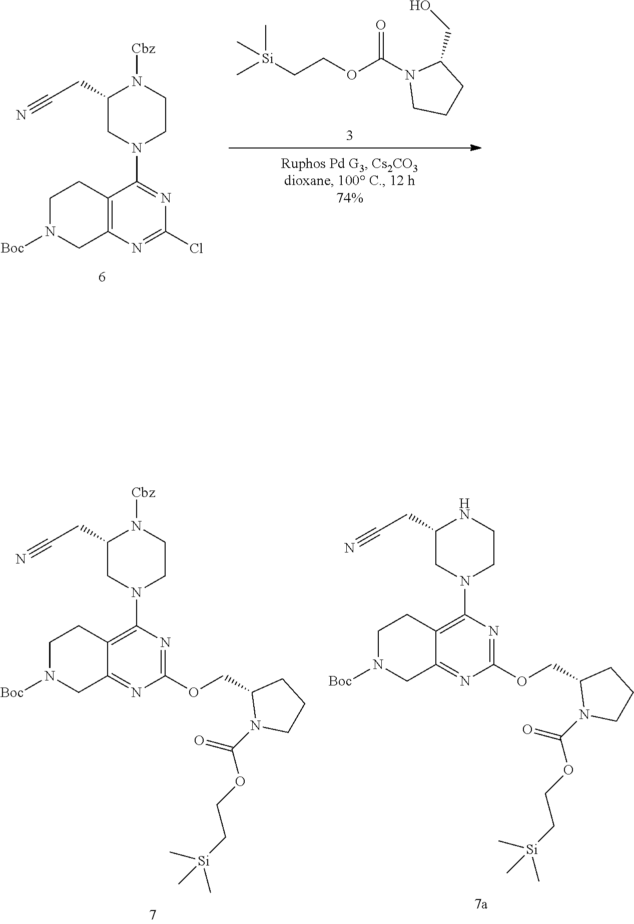 [SMILES: CC(C)(C)OC(=O)N1CCc2c(nc(Cl)nc2N2CCN(C(=O)OCc3ccccc3)[C@@H](CC#N)C2)C1 ; C[Si](C)(C)CCOC(=O)N1CCC[C@H]1CO ; "3" ; CC(C)(C)OC(=O)N1CCc2c(nc(OC[C@@H]3CCCN3C(=O)OCC[Si](C)(C)C)nc2N2CCN(C(=O)OCc3ccccc3)[C@@H](CC#N)C2)C1 ; CC(C)(C)OC(=O)N1CCc2c(nc(OC[C@@H]3CCCN3C(=O)OCC[Si](C)(C)C)nc2N2CCN[C@@H](CC#N)C2)C1]

A mixture of compound 6 (5.30 g, 10.0 mmol, 1.0 equiv, WO2017/201161A1), compound 3 (5.80 g, 23.63 mmol, 2.3 equiv), RuPhos Pd G3 (420 mg, 502 μmol, 0.05 equiv) and Cs₂CO₃ (9.83 g, 30.17 mmol, 3.0 equiv) in dioxane (60 mL) was degassed and purged with N₂ for 3 times, and then the mixture was stirred at 100° C. for 12 h under N₂ atmosphere. The reaction mixture was filtered and the filtrate was concentrated under reduced pressure to give a residue. The residue was purified by column chromatography (SiO₂, Petroleum ether/Ethyl acetate=3/1 to 1/1 to DCM/MeOH=10/1). Compound 7 (5.5 g, 7.5 mmol, 74% yield) was obtained as a yellow gum. Compound 7a (1.3 g, 2.2 mmol, 21% yield) was obtained as a yellow gum. ¹H NMR (400 MHz, CD₃OD): δ 7.52-7.24 (m, 5H), 5.24-5.12 (m, 2H), 4.57-4.43 (m, 2H), 4.40-4.28 (m, 2H), 4.24-3.90 (m, 7H), 3.81-3.63 (m, 1H), 3.51-3.35 (m, 4H), 3.21-2.81 (m, 4H), 2.69 (m, 2H), 2.16-1.91 (m, 4H), 1.49 (s, 9H), 1.10-0.86 (m, 2H), 0.04 (s, 9H). LC-MS: MS (ES⁺): RT=1.020 min, m/z=736.6 [M+H⁺].

### 5. Preparation of Compound 8

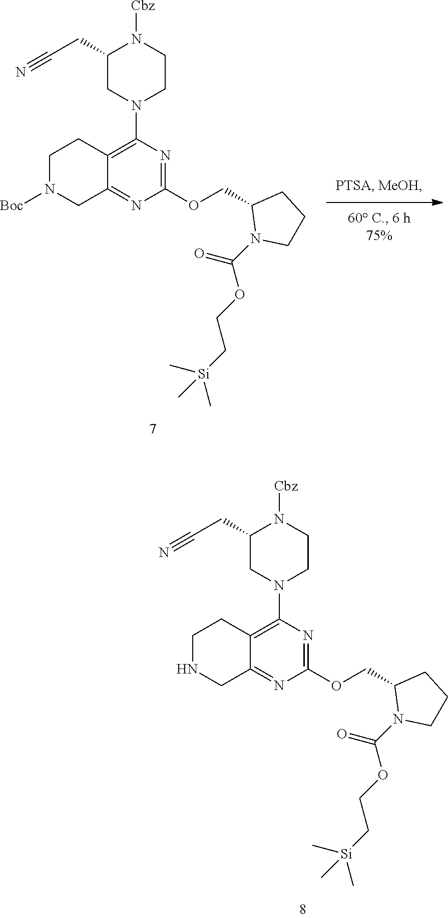 [SMILES: CC(C)(C)OC(=O)N1CCc2c(nc(OC[C@@H]3CCCN3C(=O)OCC[Si](C)(C)C)nc2N2CCN(C(=O)OCc3ccccc3)[C@@H](CC#N)C2)C1 ; C[Si](C)(C)CCOC(=O)N1CCC[C@H]1COc1nc2c(c(N3CCN(C(=O)OCc4ccccc4)[C@@H](CC#N)C3)n1)CCNC2]

To a solution of compound 7 (13.5 g, 18.3 mmol, 1.0 equiv) in MeOH (130 mL) was added PTSA (4.21 g, 22.1 mmol, 1.2 equiv). The mixture was stirred at 60° C. for 6 h. The reaction mixture was concentrated under reduced pressure to give a residue. The residue was partitioned between NaHCO₃ (50 mL) and EtOAc (200 mL). The organic phase was separated, washed with brine (50 mL), dried over Na₂SO₄, filtered and concentrated under reduced pressure to give a residue. The residue was purified by column chromatography (SiO₂, DCM:MeOH:NH₃·H₂O=10:1:0.1). Compound 8 (8.80 g, 13.8 mmol, 75% yield) was obtained as a yellow solid. ¹HNMR (400 MHz, CD₃OD): δ 7.46-7.28 (m, 5H), 5.25-5.12 (m, 2H), 4.52-4.24 (m, 2H), 4.19-3.92 (m, 6H), 3.85 (s, 2H), 3.46-3.35 (m, 3H), 3.20-2.83 (m, 5H), 2.69 (m, 2H), 2.08-1.83 (m, 4H), 1.08-0.89 (m, 2H), 0.04 (s, 9H). LC-MS: MS (ES⁺): RT=0.881 min, m/z=636.3 [M+H⁺].

### 6. Preparation of Compound 10

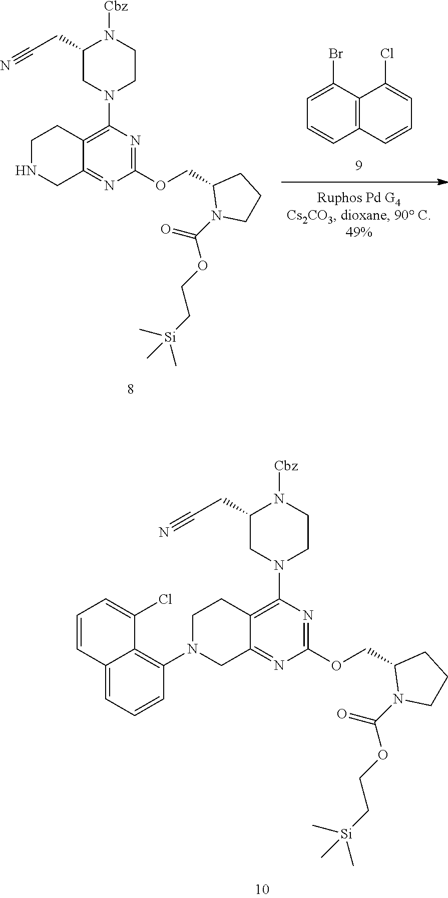 [SMILES: C[Si](C)(C)CCOC(=O)N1CCC[C@H]1COc1nc2c(c(N3CCN(C(=O)OCc4ccccc4)[C@@H](CC#N)C3)n1)CCNC2 ; Clc1cccc2cccc(Br)c12 ; C[Si](C)(C)CCOC(=O)N1CCC[C@H]1COc1nc2c(c(N3CCN(C(=O)OCc4ccccc4)[C@@H](CC#N)C3)n1)CCN(c1cccc3cccc(Cl)c13)C2]

A mixture of compound 8 (2.10 g, 3.30 mmol, 1.0 equiv), compound 9 (1.20 g, 4.95 mmol, 1.5 equiv, for US2019/144444A1), Cs₂CO₃ (3.23 g, 9.91 mmol, 3.0 equiv) and RuPhos-Pd-G4 (280 mg, 329 μmol, 0.1 equiv) in dioxane (20 mL) was degassed and purged with N₂ for 3 times, and then the mixture was stirred at 90° C. for 12 h under N₂ atmosphere. The reaction mixture was filtered and the filtrate was concentrated under reduced pressure to give a residue. The residue was purified by column chromatography (SiO₂, Petroleum ether/Ethyl acetate=2/1 to 3/2). Compound 10 (1.3 g, 1.6 mmol, 49% yield) was obtained as a yellow solid. ¹HNMR (400 MHz, CD₃OD): δ 7.81 (d, J=8.0 Hz, 1H), 7.66 (m, 1H), 7.59-7.23 (m, 9H), 5.29-5.10 (m, 2H), 4.50-3.87 (m, 10H), 3.34 (m, 6H), 3.24-2.48 (m, 7H), 2.02 (m, 2H), 1.92-1.77 (m, 1H), 1.04-0.91 (m, 2H), 0.09-0.06 (m, 9H). LC-MS: MS (ES⁺): m/z=796.3 [M+H⁺].

### 7. Preparation of Compound 11

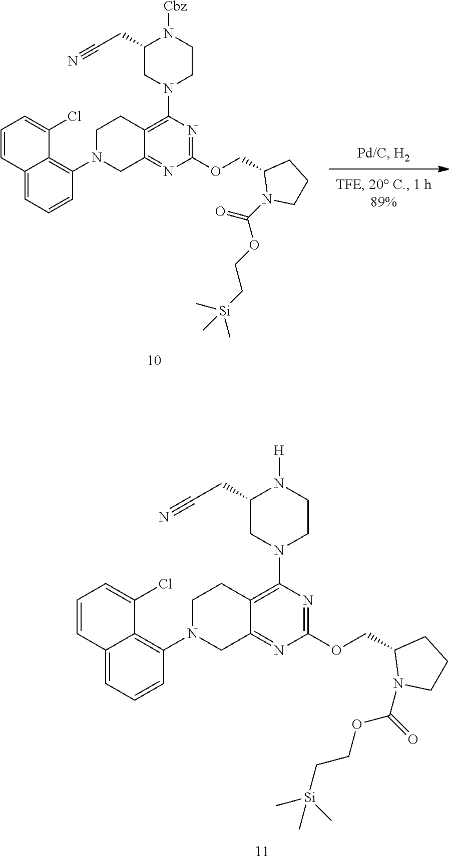 [SMILES: C[Si](C)(C)CCOC(=O)N1CCC[C@H]1COc1nc2c(c(N3CCN(C(=O)OCc4ccccc4)[C@@H](CC#N)C3)n1)CCN(c1cccc3cccc(Cl)c13)C2 ; C[Si](C)(C)CCOC(=O)N1CCC[C@H]1COc1nc2c(c(N3CCN[C@@H](CC#N)C3)n1)CCN(c1cccc3cccc(Cl)c13)C2]

A mixture of compound 10 (0.7 g, 878 μmol, 1.0 equiv), Pd/C (300 mg, 439 μmol, 10% purity, 0.50 equiv) in TFE (30 mL) was degassed and purged with H₂ for 3 times, and then the mixture was stirred at 20° C. for 1 h under H₂ atmosphere. The reaction mixture was filtered and concentrated under reduced pressure to give a residue. The crude product compound 11 (520 mg, 785 μmol, 89% yield) was used into the next step without further purification. LC-MS: MS (ES⁺): m/z=662.2 [M+H⁺].

### 8. Preparation of Compound 13

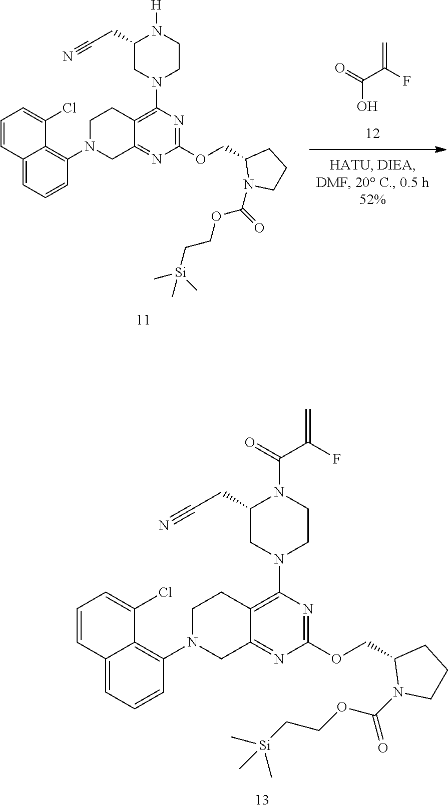

To a solution of compound 11 (520 mg, 785 μmol, 1.0 equiv) and compound 12 (106 mg, 1.18 mmol, 1.5 equiv) in DMF (4 mL) was added HATU (468 mg, 1.23 mmol, 1.5 equiv) and DIEA (207 mg, 1.61 mmol, 280 μL, 2.0 equiv). The mixture was stirred at 20° C. for 0.5 h. The reaction mixture was filtered and concentrated under reduced pressure to give a residue. The residue was purified by prep-HPLC (column: Phenomenex luna C18 150*40 mm*15 μm; mobile phase: [water (0.10% TFA)-ACN]; B %: 46%-76%, 11 min). Compound 13 (300 mg, 408 μmol, 52% yield) was obtained as a yellow solid. ¹HNMR (400 MHz, CD₃OD): δ 7.80 (d, J=7.8 Hz, 1H), 7.68 (d, J=8.2 Hz, 1H), 7.55-7.43 (m, 2H), 7.39-7.27 (m, 2H), 5.43-5.20 (m, 2H), 4.93-4.81 (m, 2H), 4.74-4.60 (m, 1H), 4.55-4.24 (m, 4H), 4.23-4.04 (m, 4H), 3.84-3.73 (m, 1H), 3.61-3.48 (m, 2H), 3.47-3.32 (m, 3H), 3.24-2.87 (m, 4H), 2.83-2.60 (m, 1H), 2.05-1.86 (m, 4H), 1.06-0.88 (m, 2H), 0.01 (s, 9H). LC-MS: MS (ES⁺): m/z=734.3 [M+H⁺].

### 9. Preparation of Compound 14

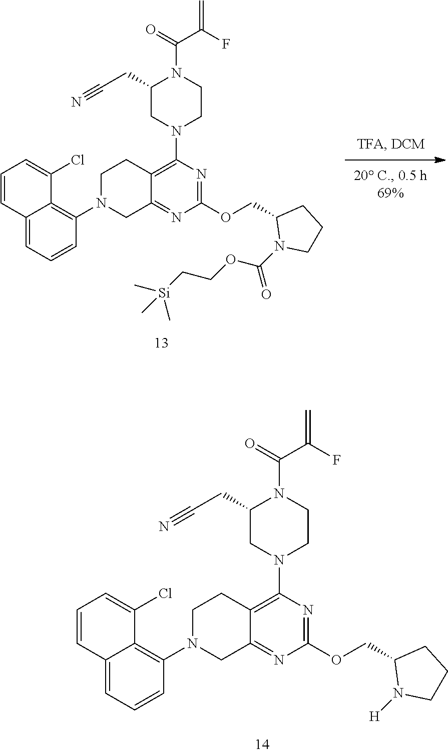 [SMILES: C=C(F)C(=O)N1CCN(c2nc(OC[C@@H]3CCCN3C(=O)OCC[Si](C)(C)C)nc3c2CCN(c2cccc4cccc(Cl)c24)C3)C[C@@H]1CC#N ; C=C(F)C(=O)N1CCN(c2nc(OC[C@@H]3CCCN3)nc3c2CCN(c2cccc4cccc(Cl)c24)C3)C[C@@H]1CC#N]

To a solution of compound 13 (300 mg, 408 μmol, 1.0 equiv) in CH₂Cl₂ (4 mL) was added TFA (1 mL). The mixture was stirred at 20° C. for 0.5 h. The reaction mixture was concentrated under reduced pressure to give a residue. The residue was purified by prep-HPLC (column: 3_Phenomenex Luna C18 75*30 mm*3 μm; mobile phase: [water (0.1% TFA)-ACN]; B %: 32%-52%, 7 min). Compound 14 (200 mg, 284 μmol, 69% yield, TFA salt) was obtained as a yellow solid. ¹HNMR (400 MHz, CD₃OD): δ 7.84 (d, J=8.1 Hz, 1H), 7.71 (d, J=8.1 Hz, 1H), 7.58-7.47 (m, 2H), 7.43-7.32 (m, 2H), 5.43-5.26 (m, 2H), 4.75 (m, 1H), 4.61-4.42 (m, 2H), 4.41-4.11 (m, 3H), 4.09-3.99 (m, 1H), 3.77 (m, 1H), 3.67-3.53 (m, 2H), 3.49-3.32 (m, 5H), 3.29-2.93 (m, 4H), 2.83-2.65 (m, 1H), 2.34-2.22 (m, 1H), 2.21-2.01 (m, 2H), 1.98-1.83 (m, 1H). LC-MS: MS (ES⁺): m/z=590.2 [M+H⁺].

### 10. Preparation of Compound I-3

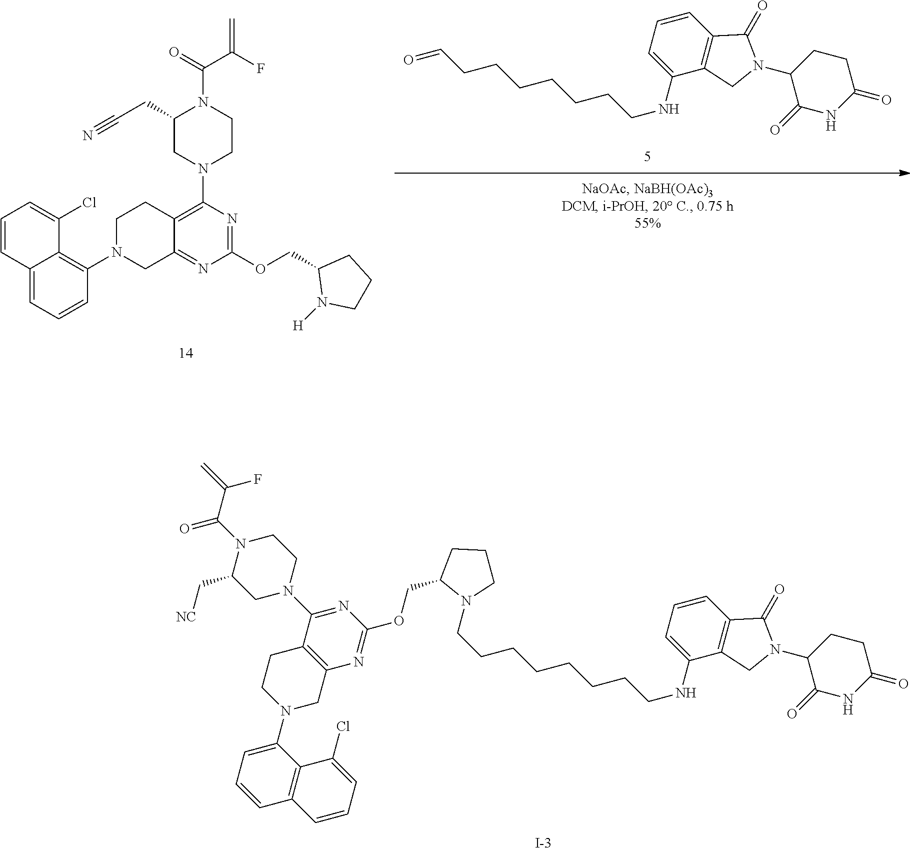

A mixture of compound 14 (45 mg, 63 μmol, 1.0 equiv, TFA salt) and NaOAc (26 mg, 316 μmol, 4.9 equiv) in DCM (2 mL) and i-PrOH (2 mL) was stirred at 20° C. for 15 min. Compound 5 (29 mg, 76 μmol, 1.2 equiv) was added, and then NaBH(OAc)₃ (67 mg, 316 μmol, 4.9 equiv) was added. The mixture was stirred at 20° C. for 0.5 h. The reaction mixture was partitioned between brine (20 mL) and DCM (80 mL). The organic phase was separated, dried over Na₂SO₄, filtered and concentrated under reduced pressure to give a residue. The residue was purified by prep-TLC (SiO₂, DCM:MeOH=10:1) to give desired compound 2-((2S)-4-(7-(8-chloronaphthalen-1-yl)-2-(((2S)-1-(8-((2-(2,6-dioxopiperidin-3-yl)-1-oxoisoindolin-4-yl)amino)octyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile (36 mg, 35 μmol, 55% yield, 99% purity, CH₃COOH salt) as a yellow solid. ¹HNMR (400 MHz, CD₃OD): δ 7.81 (d, J=8.1 Hz, 1H), 7.67 (d, J=7.9 Hz, 1H), 7.55-7.43 (m, 2H), 7.40-7.24 (m, 3H), 7.05 (d, J=7.5 Hz, 1H), 6.82-6.70 (m, 1H), 5.43-5.23 (m, 2H), 5.14 (m, 1H), 4.51-4.29 (m, 3H), 4.28-4.18 (m, 3H), 4.14-4.05 (m, 1H), 3.81-3.41 (m, 6H), 3.16-3.16 (m, 1H), 3.30-3.05 (m, 8H), 3.00-2.57 (m, 6H), 2.50-2.35 (m, 1H), 2.31-2.09 (m, 2H), 1.94 (m, 6H), 1.62 (m, J=7.0, 13.8 Hz, 4H), 1.41-1.25 (m, 9H). LC-MS: MS (ES⁺): RT=1.340 min, m/z=959.3 [M/2+H⁺]; LCMS method: LC-MS METHOD 40.M.

### Example 4—Preparation of Compound I-4

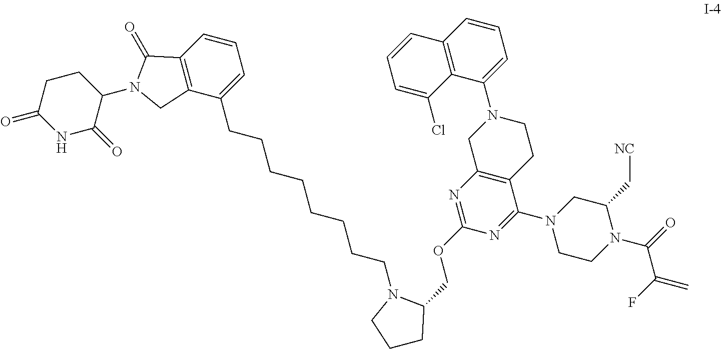 [SMILES: C=C(F)C(=O)N1CCN(c2nc(OC[C@@H]3CCCN3CCCCCCCCc3cccc4c3CN(C3CCC(=O)NC3=O)C4=O)nc3c2CCN(c2cccc4cccc(Cl)c24)C3)C[C@@H]1CC#N]

The title compound was prepared according to the following procedures.

### 1. Preparation of Compound 3

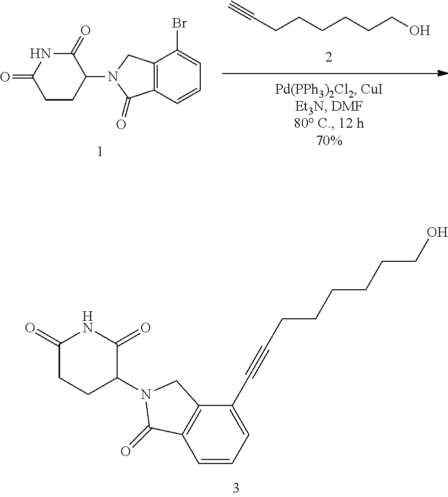

A mixture of 3-(4-bromo-1-oxo-isoindolin-2-yl)piperidine-2,6-dione (500 mg, 1.55 mmol, 1.0 equiv), oct-7-yn-1-ol (390 mg, 3.09 mmol, 2.0 equiv), Pd(PPh₃)₂Cl₂ (109 mg, 155 μmol, 0.1 equiv), Et₃N (3.64 g, 35.9 mmol, 23.2 equiv) and CuI (58.9 mg, 309 μmol, 0.2 equiv) in DMF (8 mL) was heated to 80° C. and stirred for 12 h. The reaction mixture was diluted brine (45 mL). The mixture was filtered and the filtrate was extracted with EtOAc (25 mL*3). The combined organic phase was dried over anhydrous Na₂SO₄, filtered and concentrated in vacuo. The residue was triturated with EtOH (6 mL). The resultant precipitate solid was collected by filtration and dried in vacuo to afford 3-[4-(8-hydroxyoct-1-ynyl)-1-oxo-isoindolin-2-yl]piperidine-2,6-dione (410 mg, 1.09 mmol, 70% yield) as a white solid. ¹H NMR (400 MHz, DMSO-d₆) δ 11.01 (s, 1H), 7.71 (d, 1H, J=7.6 Hz), 7.64 (d, 1H, J=7.2 Hz), 7.58-7.46 (m, 1H), 5.17-5.13 (m, 1H), 4.53-4.41 (m, 1H), 4.38-4.24 (m, 2H), 3.44-3.37 (m, 2H), 3.00-2.83 (m, 1H), 2.65-2.56 (m, 1H), 2.50-2.39 (m, 3H), 2.08-1.97 (m, 1H), 1.64-1.52 (m, 2H), 1.49-1.28 (m, 6H). LC-MS: MS (ES⁺): RT=0.833 min, m/z=369.1 [M+H⁺].

### 2. Preparation of Compound 4

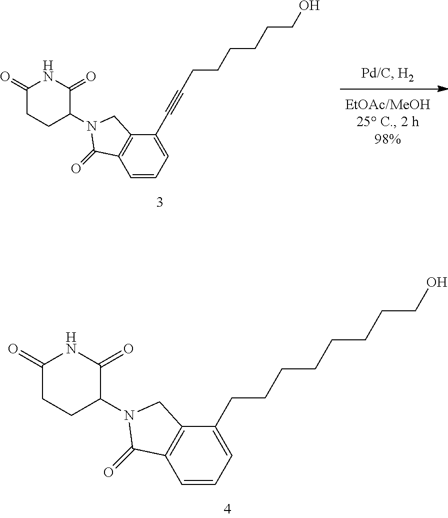 [SMILES: O=C1CCC(N2Cc3c(C#CCCCCCCO)cccc3C2=O)C(=O)N1 ; O=C1CCC(N2Cc3c(CCCCCCCCO)cccc3C2=O)C(=O)N1]

To a solution of 3-[4-(8-hydroxyoct-1-ynyl)-1-oxo-isoindolin-2-yl]piperidine-2,6-dione (360 mg, 977 μmol, 1.0 equiv) in EtOAc (5 mL) and MeOH (20 mL) was added Pd/C (100 mg, 10% purity) under N₂. The mixture was degassed with H₂ and stirred at 25° C. for 2 h under H₂ (15 psi). The reaction mixture was filtered and the filtrate was concentrated in vacuo to afford 3-[4-(8-hydroxyoctyl)-1-oxo-isoindolin-2-yl]piperidine-2,6-dione (355 mg, 953 μmol, 98% yield) as a white solid. ¹H NMR (400 MHz, DMSO-d₆) δ 11.00 (s, 1H), 7.61-7.52 (m, 1H), 7.46 (d, 2H, J=4.4 Hz), 5.16-5.12 (m, 1H), 4.54-4.40 (m, 1H), 4.36-4.21 (m, 2H), 3.41-3.35 (m, 2H), 2.99-2.87 (m, 1H), 2.67-2.62 (m, 2H), 2.47-2.38 (m, 1H), 2.09-1.96 (m, 1H), 1.55-1.65 (m, 2H), 1.46-1.35 (m, 2H), 1.34-1.19 (m, 8H). LC-MS: MS (ES⁺): RT=0.759 min, m/z=373.2 [M+H⁺].

### 3. Preparation of Compound 5

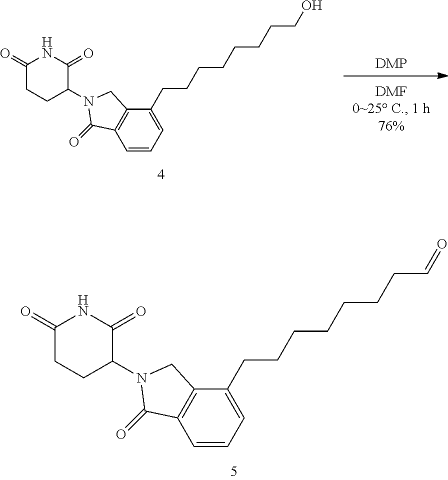

To a solution of 3-[4-(8-hydroxyoctyl)-1-oxo-isoindolin-2-yl]piperidine-2,6-dione (50.0 mg, 134 μmol, 1 equiv) in DMF (2 mL) was added DMP (85.4 mg, 201 μmol, 1.5 equiv) at 0° C. Then the mixture was warmed to 25° C. and stirred for 1 h. The reaction mixture was diluted with saturated aqueous NaHCO₃ (8 mL) and extracted with EtOAc (4 mL*3). The combined organic phase was washed with brine (5 mL), dried over anhydrous Na₂SO₄, filtered and concentrated in vacuo. The residue was purified by preparative TLC on silica gel (Dichloromethane/Methanol=10/1) to afford 8-[2-(2,6-dioxo-3-piperidyl)-1-oxo-isoindolin-4-yl]octanal (40.0 mg, 102 μmol, 76% yield) as a white solid. ¹H NMR (400 MHz, CDCl₃) δ 9.69 (s, 1H), 7.97-7.87 (m, 1H), 7.67 (d, 1H, J=7.2 Hz), 7.40-7.28 (m, 2H), 5.21-5.17 (m, 1H), 4.42-4.33 (m, 1H), 4.28-4.16 (m, 1H), 2.84-2.74 (m, 2H), 2.55 (t, 2H, J=7.6 Hz), 2.41-2.12 (m, 4H), 1.64-1.50 (m, 10H). LC-MS: MS (ES⁺): RT=0.864 min, m/z=371.1 [M+H⁺].

### 4. Preparation of Compound I-4

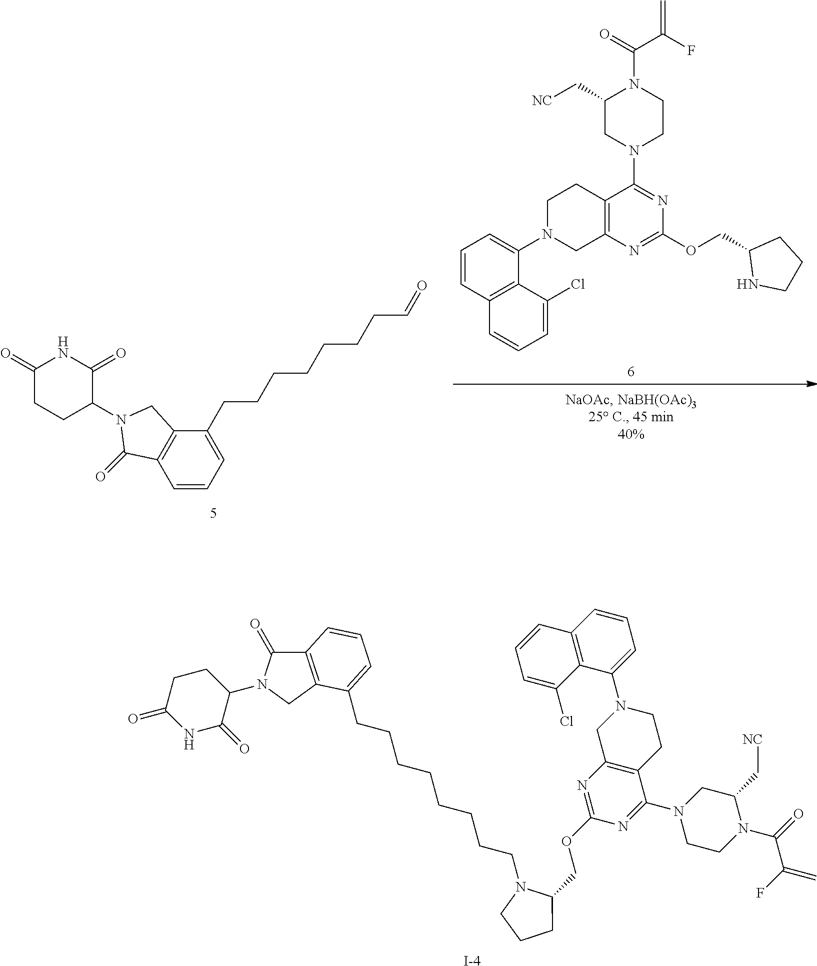

A mixture of 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (25.0 mg, 35.5 μmol, 1.0 equiv, TFA salt) and NaOAc (14.6 mg, 177 μmol, 5.0 equiv) in i-PrOH (1 mL) and DCM (1 mL) was stirred at 25° C. for 15 min. To the mixture were added 8-[2-(2,6-dioxo-3-piperidyl)-1-oxo-isoindolin-4-yl]octanal (18.2 mg, 46.2 μmol, 1.3 equiv) and NaBH(OAc)₃ (37.6 mg, 177 μmol, 5 equiv). The mixture was stirred at 25° C. for 0.5 h. The reaction mixture was diluted with water (8 mL) and extracted with DCM (5 mL*3). The combined organic phase was dried over anhydrous Na₂SO₄, filtered and concentrated in vacuo. The residue was purified by prep-HPLC (column: Phenomenex Gemini-NX C18 75*30 mm*3 μm; mobile phase: [water (0.225% FA)-ACN]; B %: 28%-58%, 7 min) to afford 2-[(2S)-4-[7-(8-chloro-1-naphthyl)-2-[[(2S)-1-[8-[2-(2,6-dioxo-3-piperidyl)-1-oxo-isoindolin-4-yl]octyl]pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (14.0 mg, 14.2 μmol, 40% yield, 96% purity) as a white solid. ¹H NMR (400 MHz, DMSO-d₆) δ 11.00 (s, 1H), 7.91 (d, 1H, J=8.8 Hz), 7.78-7.70 (m, 1H), 7.59-7.49 (m, 3H), 7.46-7.30 (m, 4H), 5.44-5.35 (m, 1H), 5.35-5.18 (m, 1H), 5.15-5.11 (m, 1H), 4.48-3.82 (m, 8H), 3.80-3.67 (m, 1H), 3.55-3.46 (m, 1H), 3.28-3.16 (m, 4H), 3.13-3.02 (m, 4H), 2.99-2.86 (m, 3H), 2.76-2.68 (m, 1H), 2.65-2.56 (m, 3H), 2.47-2.34 (m, 3H), 2.06-1.96 (m, 2H), 1.86-1.65 (m, 3H), 1.59-1.46 (m, 4H), 1.32-1.14 (m, 9H). LC-MS: MS (ES⁺): RT=1.439 min, m/z=944.3 [M+H⁺]; LCMS method: LC-MS METHOD 40.

### Example 5—Preparation of Compound I-5

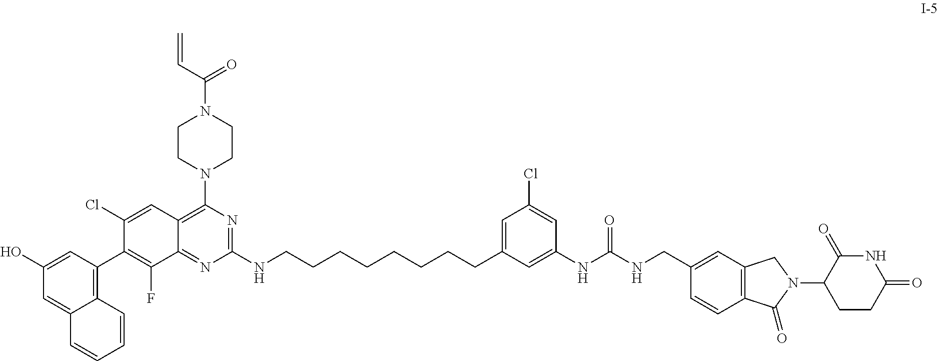 [SMILES: C=CC(=O)N1CCN(c2nc(NCCCCCCCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

The title compound was prepared according to the following procedures.

### 1. Preparation of Compound 3

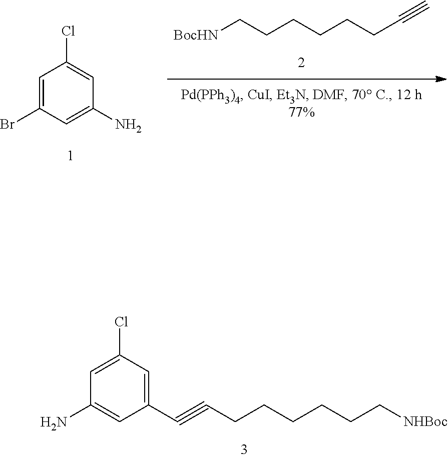 [SMILES: Nc1cc(Cl)cc(Br)c1 ; C#CCCCCCCNC(=O)OC(C)(C)C ; CC(C)(C)OC(=O)NCCCCCCC#Cc1cc(N)cc(Cl)c1]

To a solution of 3-bromo-5-chloro-aniline (458 mg, 2.20 mmol, 1.0 equiv) and tert-butyl N-oct-7-ynylcarbamate (0.50 g, 2.2 mmol, 1.0 equiv) in DMF (5 mL) was added Pd(PPh₃)₂Cl₂ (156 mg, 220 μmol, 0.1 equiv), Et₃N (0.67 g, 6.7 mmol, 0.93 mL, 3.0 equiv) and CuI (43 mg, 0.22 mmol, 0.1 equiv), and then it was stirred at 70° C. for 12 h under N₂. The reaction mixture was poured into 50 mL water and then it was extracted with EtOAc (2×20 mL). The organic layers were dried over anhydrous Na₂SO₄, filtered and concentrated to afford crude product. The residue was purified by silica chromatography (Petroleum ether/Ethyl acetate=5/1 to 3/1) to afford tert-butyl N-[8-(3-amino-5-chloro-phenyl)oct-7-ynyl]carbamate (600 mg, 1.71 mmol, 77% yield) as a brown oil. LC-MS: MS (ES⁺): m/z=351.2 [M+H⁺].

### 2. Preparation of Compound 4

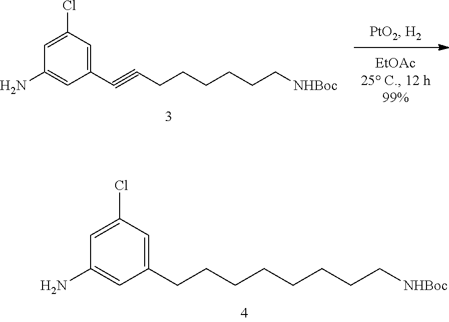 [SMILES: CC(C)(C)OC(=O)NCCCCCCC#Cc1cc(N)cc(Cl)c1 ; CC(C)(C)OC(=O)NCCCCCCCCc1cc(N)cc(Cl)c1]

To a solution of tert-butyl N-[8-(3-amino-5-chloro-phenyl)oct-7-ynyl]carbamate (600 mg, 1.71 mmol, 1.0 equiv) in EtOAc (20 mL) was added PtO₂ (150 mg, 661 μmol, 0.39 equiv), and then it was degassed and purged with H₂. The reaction mixture was stirred at 25° C. for 12 h under 15 psi. The reaction mixture was filtered and the filtrate was concentrated to afford tert-butyl N-[8-(3-amino-5-chloro-phenyl)octyl]carbamate (600 mg, 1.69 mmol, 99% yield) as a brown oil and used for the next step directly. ¹H NMR: (400 MHz, CDCl₃) δ 6.61 (s, 1H), 6.56 (s, 1H), 6.43 (s, 1H), 4.51 (brs, 1H), 3.11 (m, 4H), 2.51-2.44 (m, 2H), 1.61-1.52 (m, 2H), 1.45 (s, 19H). LC-MS: MS (ES⁺): m/z=355.5 [M+H⁺].

### 3. Preparation of Compound 6

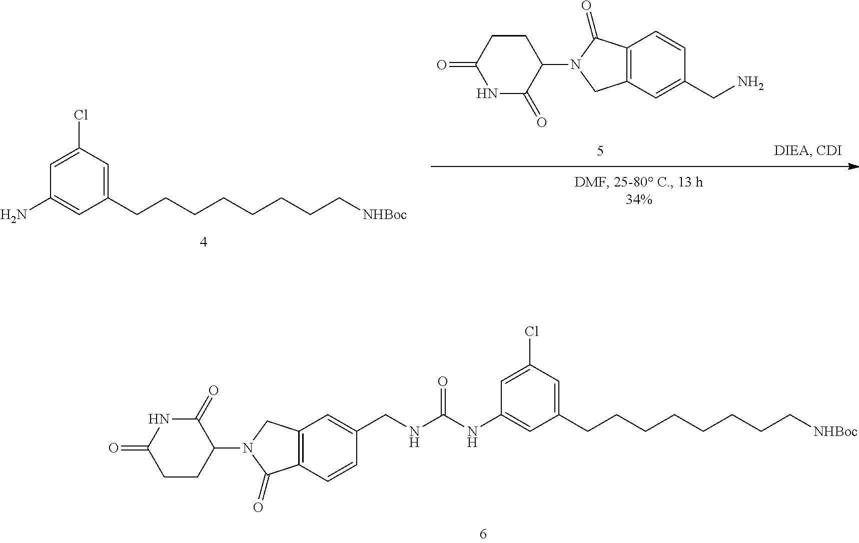 [SMILES: CC(C)(C)OC(=O)NCCCCCCCCc1cc(N)cc(Cl)c1 ; NCc1ccc2c(c1)CN(C1CCC(=O)NC1=O)C2=O ; CC(C)(C)OC(=O)NCCCCCCCCc1cc(Cl)cc(NC(=O)NCc2ccc3c(c2)CN(C2CCC(=O)NC2=O)C3=O)c1]

To a solution of 3-[5-(aminomethyl)-1-oxo-isoindolin-2-yl]piperidine-2,6-dione (786 mg, 2.54 mmol, 1.50 eq, HCl) in DMF (10 mL) was added DIEA (0.87 g, 6.8 mmol, 1.2 mL, 4.0 equiv) and CDI (617 mg, 3.80 mmol, 2.25 equiv), and then it was stirred at 25° C. for 1 h. Tert-butyl N-[8-(3-amino-5-chloro-phenyl)octyl]carbamate (600 mg, 1.69 mmol, 1.0 equiv) was added to the mixture and then it was stirred at 80° C. for 12 h. The residue was quenched by 50 mL water, and then it was extracted with EtOAc (3×20 mL). The organic layers were dried over anhydrous Na₂SO₄, filtered and concentrated to afford crude product. The residue was purified by silica chromatography (CH₂Cl₂:MeOH=20:1-10:1) to afford tert-butyl N-[8-[3-chloro-5-[[2-(2,6-dioxo-3-piperidyl)-1-oxo-isoindolin-5-yl]methylcarbamoylamino]phenyl]octyl]carbamate (380 mg, 581 μmol, 34% yield) as a yellow oil. LC-MS: MS (ES⁺): RT=1.025 min, m/z=554.2 [M−99]⁺.

### 4. Preparation of Compound 7

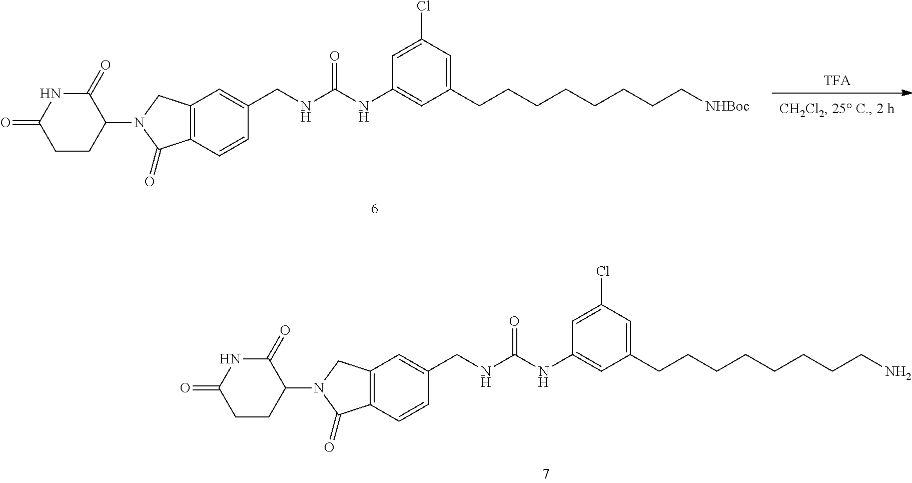 [SMILES: CC(C)(C)OC(=O)NCCCCCCCCc1cc(Cl)cc(NC(=O)NCc2ccc3c(c2)CN(C2CCC(=O)NC2=O)C3=O)c1 ; "TFA" ; NCCCCCCCCc1cc(Cl)cc(NC(=O)NCc2ccc3c(c2)CN(C2CCC(=O)NC2=O)C3=O)c1]

To a solution of tert-butyl N-[8-[3-chloro-5-[[2-(2,6-dioxo-3-piperidyl)-1-oxo-isoindolin-5-yl]methylcarbamoylamino]phenyl]octyl]carbamate (380 mg, 581 μmol, 1.0 equiv) in CH₂Cl₂ (2 mL) was added TFA (1 mL), and then it was stirred at 25° C. for 2 h. The reaction mixture was concentrated to afford 1-[3-(8-aminooctyl)-5-chloro-phenyl]-3-[[2-(2,6-dioxo-3-piperidyl)-1-oxo-isoindolin-5-yl]methyl]urea (390 mg, crude, TFA salt) as a yellow oil and used for the next step directly. LC-MS: MS (ES⁺): m/z=554.1 [M+H⁺].

### 5. Preparation of Compound 9

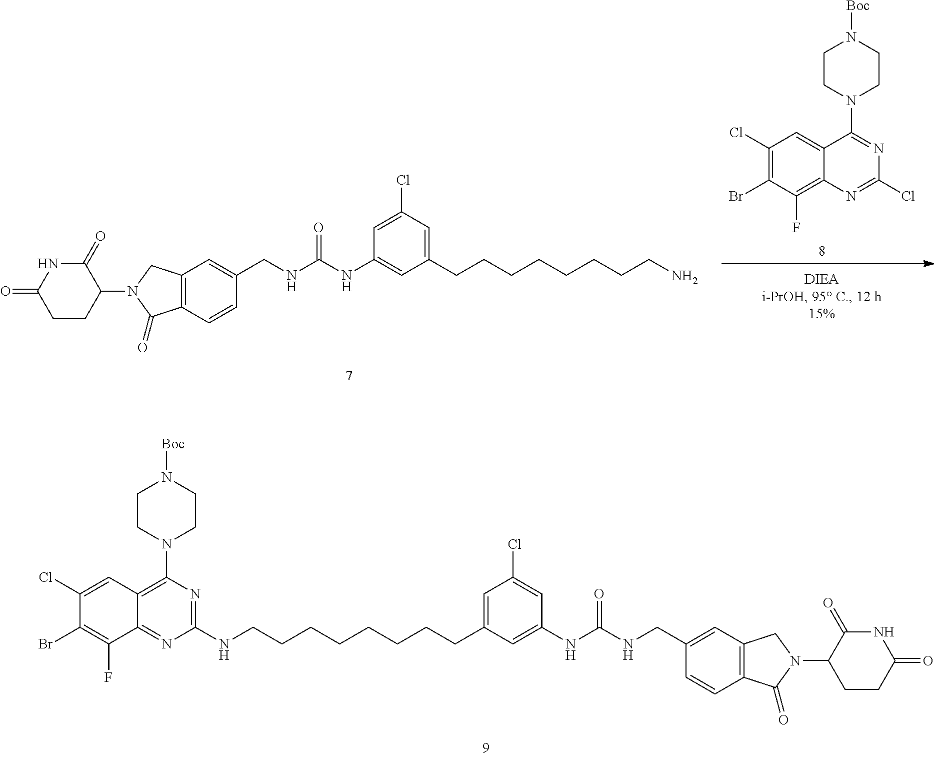

To a solution of tert-butyl 4-(7-bromo-2,6-dichloro-8-fluoro-quinazolin-4-yl)piperazine-1-carboxylate (280 mg, 584 μmol, 1.0 equiv) and 1-[3-(8-aminooctyl)-5-chloro-phenyl]-3-[[2-(2,6-dioxo-3-piperidyl)-1-oxo-isoindolin-5-yl]methyl]urea (390 mg, 584 μmol, 1.0 equiv, TFA salt) in i-PrOH (6 mL) was added DIEA (377 mg, 2.92 mmol, 510 μL, 5.0 equiv), and then it was stirred at 95° C. for 12 h. The reaction mixture was concentrated to afford crude product. The residue was purified by silica chromatography (CH₂Cl₂:MeOH=50:1-20:1) and prep-TLC (CH₂Cl₂:MeOH=10:1) to afford tert-butyl 4-[7-bromo-6-chloro-2-[8-[3-chloro-5-[[2-(2,6-dioxo-3-piperidyl)-1-oxo-isoindolin-5-yl]methylcarbamoylamino]phenyl]octylamino]-8-fluoro-quinazolin-4-yl]piperazine-1-carboxylate (86 mg, 86 μmol, 15% yield) as a yellow foam. LC-MS: MS (ES⁺): RT=0.972 min, m/z=998.6 [M+H⁺].

### 6. Preparation of Compound 11

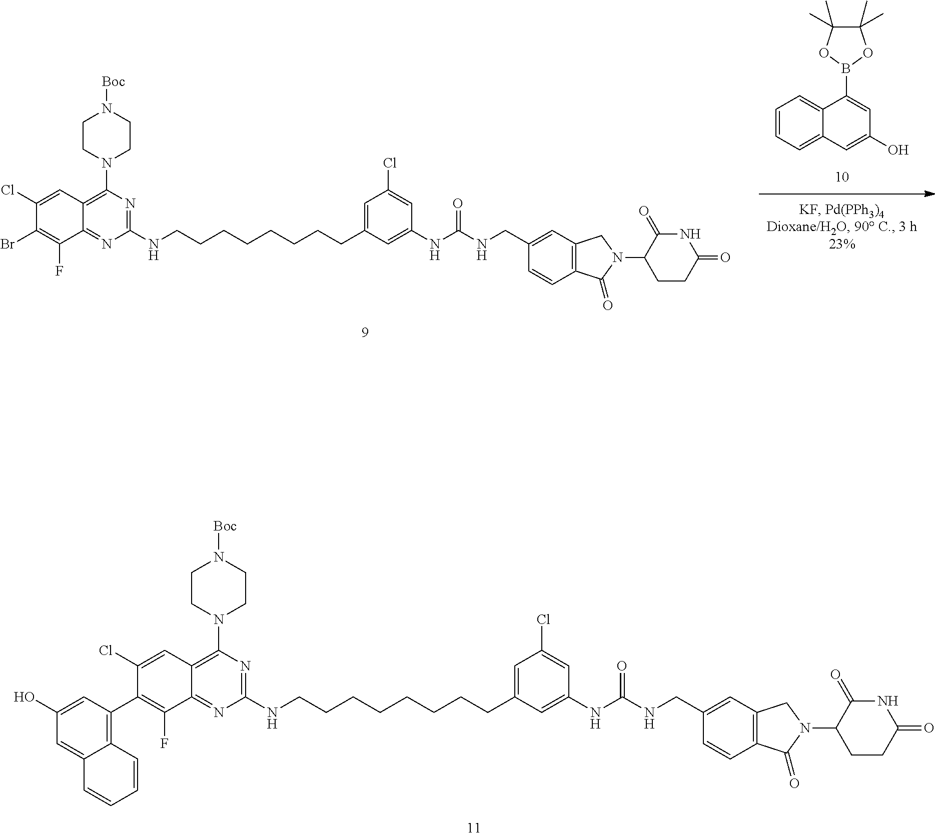 [SMILES: CC(C)(C)OC(=O)N1CCN(c2nc(NCCCCCCCCc3cc(Cl)cc(NC(=O)NCc4ccc5c(c4)CN(C4CCC(=O)NC4=O)C5=O)c3)nc3c(F)c(Br)c(Cl)cc23)CC1 ; CC1(C)OB(c2cc(O)cc3ccccc23)OC1(C)C ; CC(C)(C)OC(=O)N1CCN(c2nc(NCCCCCCCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

To a solution of tert-butyl 4-[7-bromo-6-chloro-2-[8-[3-chloro-5-[[2-(2,6-dioxo-3-piperidyl)-1-oxo-isoindolin-5-yl]methylcarbamoylamino]phenyl]octylamino]-8-fluoro-quinazolin-4-yl]piperazine-1-carboxylate (0.11 g, 0.11 mmol, 1.0 equiv) and 4-(4,4,5,5-tetramethyl-1,3,2-dioxaborolan-2-yl)naphthalen-2-ol (45 mg, 0.17 mmol, 1.5 equiv) in dioxane (5 mL) and H₂O (1 mL) was added KF (16 mg, 0.28 mmol, 2.5 equiv) and Pd(PPh₃)₄ (64 mg, 55 μmol, 0.5 equiv), and then it was stirred at 90° C. for 3 h under N₂. The reaction mixture was added 50 mL water, and then it was extracted with EtOAc (2×20 mL). The organic layers were dried over anhydrous Na₂SO₄, filtered and concentrated to afford crude product. The residue was purified by prep-TLC (CH₂Cl₂:MeOH=10:1) to afford tert-butyl 4-[6-chloro-2-[8-[3-chloro-5-[[2-(2,6-dioxo-3-piperidyl)-1-oxo-isoindolin-5-yl]methylcarbamoylamino]phenyl]octylamino]-8-fluoro-7-(3-hydroxy-1-naphthyl)quinazolin-4-yl]piperazine-1-carboxylate (27 mg, 25 μmol, 23% yield) as a yellow solid. LC-MS: MS (ES⁺): m/z=1060.5 [M+H⁺].

### 7. Preparation of Compound 12

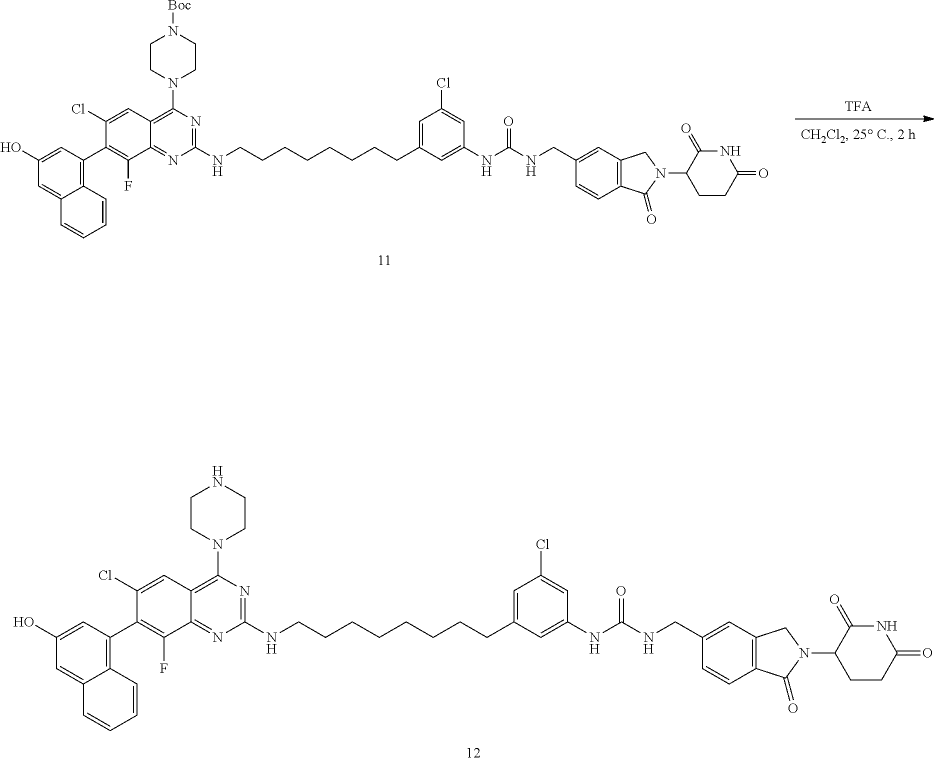 [SMILES: CC(C)(C)OC(=O)N1CCN(c2nc(NCCCCCCCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1 ; O=C1CCC(N2Cc3cc(CNC(=O)Nc4cc(Cl)cc(CCCCCCCCNc5nc(N6CCNCC6)c6cc(Cl)c(-c7cc(O)cc8ccccc78)c(F)c6n5)c4)ccc3C2=O)C(=O)N1 ; "TFA"]

To a solution of tert-butyl 4-[6-chloro-2-[8-[3-chloro-5-[[2-(2,6-dioxo-3-piperidyl)-1-oxo-isoindolin-5-yl]methylcarbamoylamino]phenyl]octylamino]-8-fluoro-7-(3-hydroxy-1-naphthyl)quinazolin-4-yl]piperazine-1-carboxylate (30 mg, 28 μmol, 1.0 equiv) in CH₂Cl₂ (1 mL) was added TFA (0.5 mL), and then it was stirred at 25° C. for 1 h. The reaction mixture was concentrated to afford tert-butyl 4-[6-chloro-2-[8-[3-chloro-5-[[2-(2,6-dioxo-3-piperidyl)-1-oxo-isoindolin-5-yl]methylcarbamoylamino]phenyl]octylamino]-8-fluoro-7-(3-hydroxy-1-naphthyl)quinazolin-4-yl]piperazine-1-carboxylate (33 mg, 28 μmol, 99% yield, TFA salt) as a yellow oil and used for the next step directly. LC-MS: MS (ES⁺): m/z=959.8 [M+H⁺].

### 8. Preparation of I-5

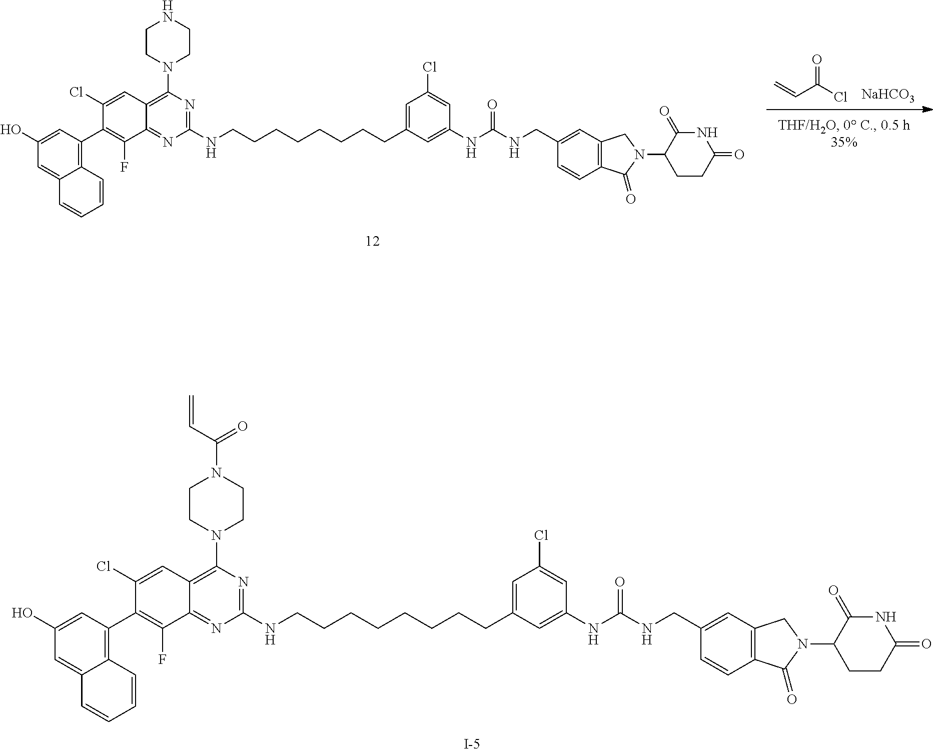

To a solution of tert-butyl 4-[6-chloro-2-[8-[3-chloro-5-[[2-(2,6-dioxo-3-piperidyl)-1-oxo-isoindolin-5-yl]methylcarbamoylamino]phenyl]octylamino]-8-fluoro-7-(3-hydroxy-1-naphthyl)quinazolin-4-yl]piperazine-1-carboxylate (33 mg, 28 μmol, 1.0 equiv, TFA salt) in THF (1 mL) was added NaHCO₃ (24 mg, 0.28 mmol, 10.0 equiv) in H₂O (0.2 mL), and then prop-2-enoyl chloride (3.0 mg, 28 μmol, 1.0 equiv) was slowly added to the mixture at 0° C. The resulting solution was stirred at 0° C. for 0.5 h. The reaction mixture was quenched by 20 mL water, and then it was extracted with EtOAc (2×20 mL). The organic layers were washed with brine (20 mL), and then dried over anhydrous Na₂SO₄, filtered and concentrated to afford crude product. The residue was purified by prep-HPLC (column: Unisil 3-100 C18 Ultra 150*50 mm*3 μm; mobile phase: [water (0.225% FA)-ACN]; B %: 29%-59%, 10 min) to afford 1-[3-chloro-5-[8-[[6-chloro-8-fluoro-7-(3-hydroxy-1-naphthyl)-4-(4-prop-2-enoylpiperazin-1-yl)quinazolin-2-yl]amino]octyl]phenyl]-3-[[2-(2,6-dioxo-3-piperidyl)-1-oxo-isoindolin-5-yl]methyl]urea (10.5 mg, 9.90 μmol, 35% yield, FA salt) as a white solid. ¹H NMR: (400 MHz, CD₃OD) δ 7.83 (s, 1H), 7.79-7.71 (m, 2H), 7.54 (s, 1H), 7.49 (d, J=7.9 Hz, 1H), 7.41 (t, J=7.4 Hz, 1H), 7.36 (s, 1H), 7.31-7.25 (m, 2H), 7.24-7.16 (m, 1H), 7.12-7.03 (m, 2H), 6.87-6.77 (m, 2H), 6.28 (dd, J=16 Hz, 1H), 5.81 (d, J=10.4 Hz, 1H), 5.14 (dd, J=12 Hz, 1H), 4.51 (s, 2H), 4.45 (d, J=4 Hz, 2H), 3.97-3.82 (m, 8H), 3.55-3.44 (m, 2H), 2.95-2.73 (m, 2H), 2.58-2.49 (m, 2H), 1.70-1.59 (m, 4H), 1.45-1.28 (m, 10H). LC-MS: MS (ES⁺): RT=3.034 min, m/z=1014.3 [M+H⁺], LCMS method: LC-MS METHOD 01.

### Example 6—Preparation of Compound I-6

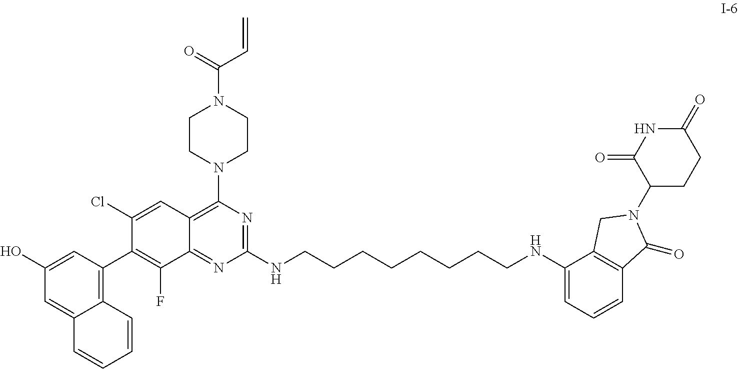 [SMILES: C=CC(=O)N1CCN(c2nc(NCCCCCCCCNc3cccc4c3CN(C3CCC(=O)NC3=O)C4=O)nc3c(F)c(-c4cc(O)cc5ccccc45)c(Cl)cc23)CC1]

The title compound was prepared according to the following procedures.

### 1. Preparation of Compound 2

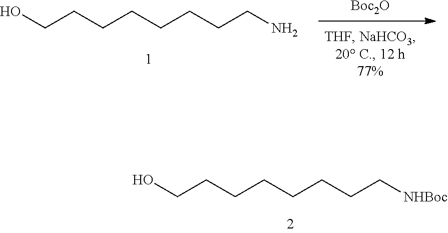 [SMILES: "Boc2O" ; NCCCCCCCCO ; CC(C)(C)OC(=O)NCCCCCCCCO]

To a solution of compound 1 (1.00 g, 6.89 mmol, 1.0 equiv) in THF (6 mL) and saturated NaHCO₃ (6 mL) was added Boc₂O (3.01 g, 13.77 mmol, 3.16 mL, 2.0 equiv). The mixture was stirred at 20° C. for 12 h. The reaction mixture was diluted with H₂O (30 mL) and extracted with EA (2×30 mL). The combined organic layers were washed with brine (3×10 mL), dried over Na₂SO₄, filtered and concentrated under reduced pressure to give a residue. The residue was purified by column chromatography (SiO₂, Petroleum ether/Ethyl acetate=10/1 to 3/1). Compound 2 (1.30 g, 5.30 mmol, 77% yield) was obtained as a white solid. ¹HNMR (400 MHz, DMSO-d₆): δ 6.75-6.73 (m, 1H), 4.34-4.27 (m, 1H), 3.39-3.34 (m, 2H), 2.93-2.83 (m, 2H), 1.37 (s, 12H), 1.29-1.15 (m, 9H).

### 2. Preparation of Compound 3

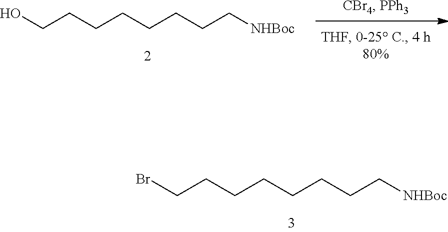

To a solution of PPh₃ (2.78 g, 10.60 mmol, 2.0 equiv) in THF (10 mL) was added CBr₄ (3.51 g, 10.60 mmol, 2.0 equiv) at 0° C. Then compound 2 (1.30 g, 5.30 mmol, 1.0 equiv) was added at 0° C. The mixture was stirred at 25° C. for 4 h. The reaction mixture was concentrated under reduced pressure to give a residue. The residue was purified by column chromatography (SiO₂, Petroleum ether/Ethyl acetate=1/0 to 5/1). Compound 3 (1.30 g, 4.22 mmol, 80% yield) was obtained as a colorless oil. ¹HNMR (400 MHz, CDCl₃): δ 4.50 (m, 1H), 3.47-3.36 (m, 2H), 3.17-3.05 (m, 2H), 1.92-1.79 (m, 2H), 1.66-1.53 (m, 1H), 1.45 (s, 13H), 1.32 (s, 6H).

### 3. Preparation of Compound 5

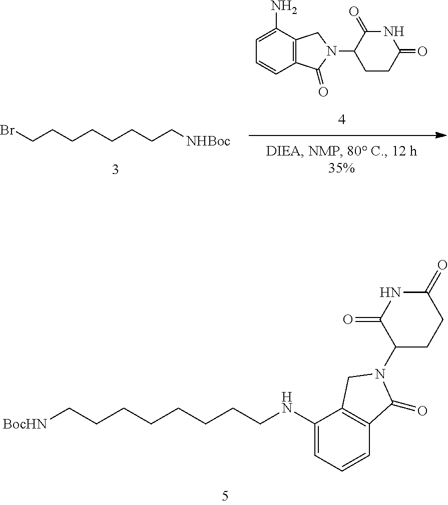 [SMILES: Nc1cccc2c1CN(C1CCC(=O)NC1=O)C2=O ; CC(C)(C)OC(=O)NCCCCCCCCBr ; CC(C)(C)OC(=O)NCCCCCCCCNc1cccc2c1CN(C1CCC(=O)NC1=O)C2=O]

To a solution of compound 3 (1.30 g, 4.22 mmol, 1.0 equiv) and compound 4 (1.09 g, 4.22 mmol, 1.0 equiv) in NMP (10 mL) was added DIEA (817.59 mg, 6.33 mmol, 1.10 mL, 1.5 equiv). The mixture was stirred at 80° C. for 12 h. The reaction mixture was diluted with H₂O (50 mL) and then filtered. The filter cake was concentrated under reduced pressure. The residue was purified by prep-HPLC (column: Phenomenex luna C18 250*50 mm*10 μm; mobile phase: [water (0.225% FA)-ACN]; B %: 35%-65%, 19 min). Compound 5 (720 mg, 1.48 mmol, 35% yield) was obtained as a white solid. ¹HNMR (400 MHz, DMSO-d₆): δ 11.00 (s, 1H), 7.33-7.22 (m, 1H), 6.92 (d, J=7.5 Hz, 1H), 6.73 (m 2H), 5.55 (brs, 1H), 5.19-5.05 (m, 1H), 4.29-4.04 (m, 2H), 3.16-3.05 (m, 2H), 2.99-2.84 (m, 3H), 2.70-2.57 (m, 1H), 2.39-2.25 (m, 1H), 2.11-1.97 (m, 1H), 1.64-1.46 (m, 2H), 1.41-1.15 (m, 20H). LC-MS: MS (ES⁺): RT=0.856 min, m/z=487.4 [M+H⁺].

### 4. Preparation of Compound 6

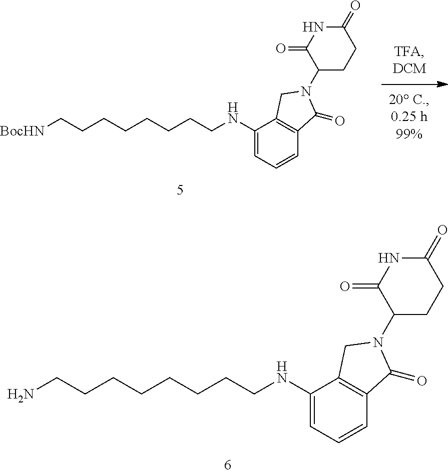 [SMILES: CC(C)(C)OC(=O)NCCCCCCCCNc1cccc2c1CN(C1CCC(=O)NC1=O)C2=O ; NCCCCCCCCNc1cccc2c1CN(C1CCC(=O)NC1=O)C2=O]

To a solution of compound 5 (720 mg, 1.48 mmol, 1.0 equiv) in CH₂Cl₂ (10 mL) was added TFA (2.5 mL). The mixture was stirred at 20° C. for 0.25 h. The reaction mixture was concentrated under reduced pressure to give a residue. Compound 6 (740 mg, 1.48 mmol, 99% yield, TFA) was obtained as a yellow oil and used for next step without purification. LC-MS: MS (ES⁺): m/z=387.0 [M+H⁺].

### 5. Preparation of Compound 8

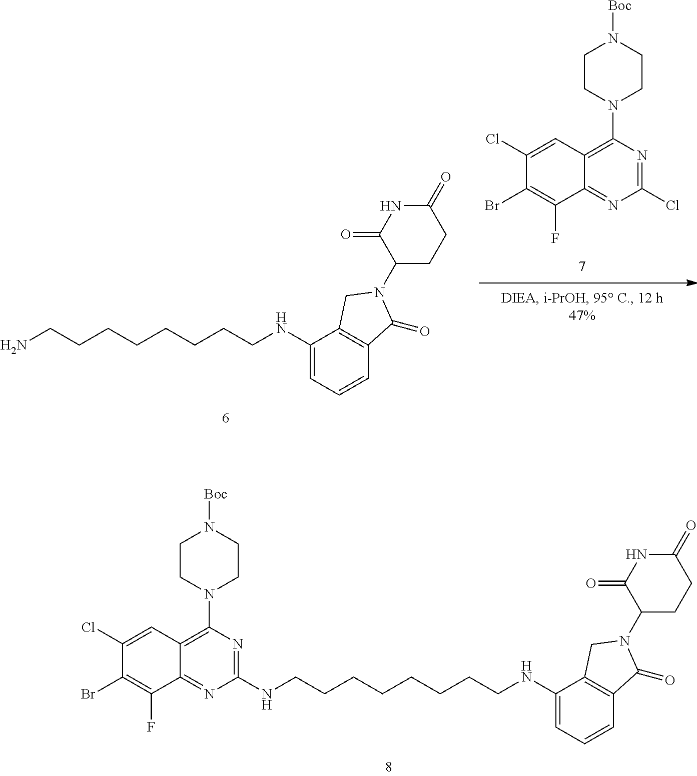 [SMILES: NCCCCCCCCNc1cccc2c1CN(C1CCC(=O)NC1=O)C2=O ; CC(C)(C)OC(=O)N1CCN(c2nc(Cl)nc3c(F)c(Br)c(Cl)cc23)CC1 ; CC(C)(C)OC(=O)N1CCN(c2nc(NCCCCCCCCNc3cccc4c3CN(C3CCC(=O)NC3=O)C4=O)nc3c(F)c(Br)c(Cl)cc23)CC1]

To a solution of compound 6 (740 mg, 1.91 mmol, 1.0 equiv) and DIEA (742 mg, 5.74 mmol, 1.00 mL, 3.0 equiv) in i-PrOH (12 mL) was added compound 7 (919 mg, 1.91 mmol, 1.0 equiv). The mixture was stirred at 95° C. for 12 h. The reaction mixture was concentrated under reduced pressure. The residue was purified by column chromatography (SiO₂, Petroleum ether/Ethyl acetate=5/1 to 1/2). Compound 8 (750 mg, 903 μmol, 47% yield) was obtained as a yellow solid. ¹HNMR (400 MHz, DMSO-d₆): δ 11.00 (s, 1H), 7.71 (s, 1H), 7.29-7.23 (m, 1H), 6.91 (d, J=7.3 Hz, 1H), 6.71 (d, J=8.0 Hz, 1H), 5.59-5.49 (m, 1H), 5.16-5.04 (m, 1H), 4.25-4.08 (m, 2H), 4.06-3.99 (m, 2H), 3.72-3.46 (m, 9H), 3.15-3.06 (m, 2H), 3.00-2.85 (m, 1H), 2.68-2.57 (m, 1H), 2.37-2.23 (m, 1H), 2.08-1.95 (m, 1H), 1.55 (m 5H), 1.46-1.25 (m, 20H). LC-MS: MS (ES⁺): RT=0.939 min, m/z=831.3 [M+H⁺].

### 6. Preparation of Compound 10

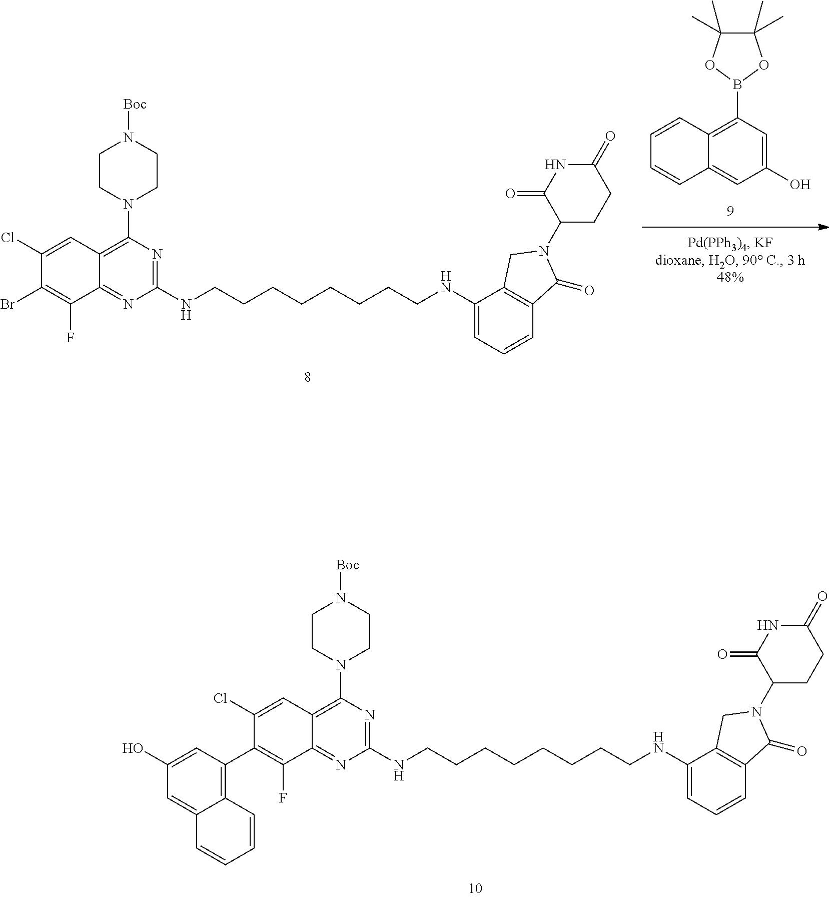

A mixture of compound 9 (171 mg, 632 μmol, 1.5 equiv), compound 8 (350 mg, 422 μmol, 1.0 equiv), Pd(PPh₃)₄ (244 mg, 211 μmol, 0.5 equiv), KF (61 mg, 1.05 mmol, 24 μL, 2.5 equiv) in dioxane (34 mL) and H₂O (6.8 mL) was degassed and purged with N₂ for 3 times, and then the mixture was stirred at 90° C. for 3 h under N₂ atmosphere. The reaction mixture was diluted with brine (40 mL) and extracted with EA (2×50 mL). The combined organic layers were washed with brine (40 mL), dried over Na₂SO₄, filtered and concentrated under reduced pressure to give a residue. The residue was purified by prep-TLC (SiO₂, DCM:MeOH=10:1). Compound 10 (180 mg, 201 μmol, 48% yield) was obtained as a yellow solid. LC-MS: MS (ES⁺): m/z=893.4 [M+H⁺].

### 7. Preparation of Compound 11

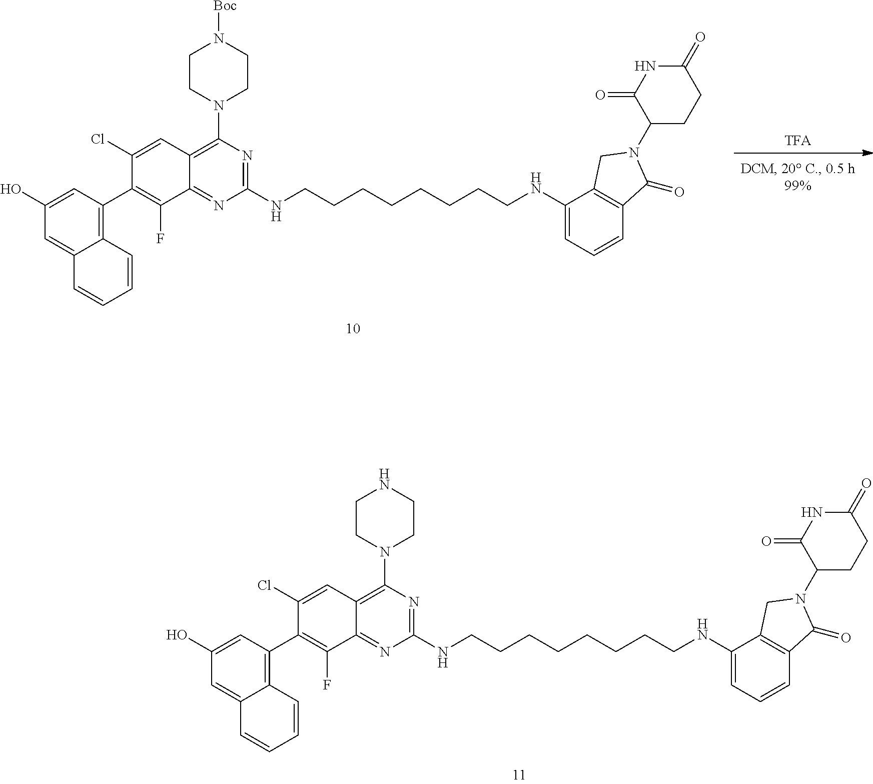 [SMILES: CC(C)(C)OC(=O)N1CCN(c2nc(NCCCCCCCCNc3cccc4c3CN(C3CCC(=O)NC3=O)C4=O)nc3c(F)c(-c4cc(O)cc5ccccc45)c(Cl)cc23)CC1 ; O=C1CCC(N2Cc3c(NCCCCCCCCNc4nc(N5CCNCC5)c5cc(Cl)c(-c6cc(O)cc7ccccc67)c(F)c5n4)cccc3C2=O)C(=O)N1 ; "TFA"]

To a solution of compound 10 (180 mg, 129 μmol, 64% purity, 1.0 equiv) in CH₂C12 (4 mL) was added TFA (1 mL). The mixture was stirred at 20° C. for 0.5 h. The reaction mixture was concentrated under reduced pressure. Compound 11 (116 mg, 128 μmol, 99% yield, TFA) was obtained as a yellow oil and used for next step without purification. LC-MS: MS (ES⁺): m/z=793.3 [M+H⁺].

### 8. Preparation of I-6

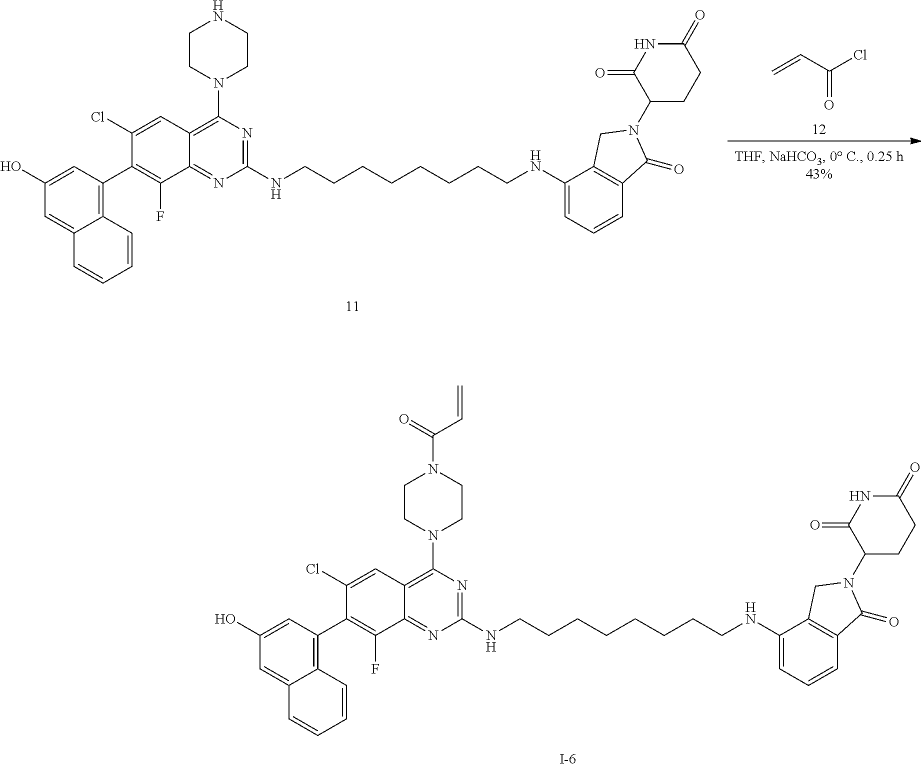

To a solution of compound 11 (116 mg, 128 μmol, 1.0 equiv, TFA) in THF (5 mL) and saturated NaHCO₃ (2.5 mL) was added a solution of compound 12 (5.79 mg, 63.9 μmol, 5.21 μL, 0.5 equiv) in THE (0.2 mL) at 0° C. The mixture was stirred at 0° C. for 0.25 h. The reaction mixture was diluted with brine 20 mL and extracted with EA (2×20 mL). The combined organic layers were washed with brine (20 mL), dried over Na₂SO₄, filtered and concentrated under reduced pressure to give a residue. The residue was purified by prep-HPLC (column: Unisil 3-100 C18 Ultra 150*50 mm*3 μm; mobile phase: [water (0.225% FA)-ACN]; B %: 25%-55%, 10 min) to give the desired product 3-(4-((8-((4-(4-acryloylpiperazin-1-yl)-6-chloro-8-fluoro-7-(3-hydroxynaphthalen-1-yl)quinazolin-2-yl)amino)octyl)-amino)-1-oxoisoindolin-2-yl)piperidine-2,6-dione (49.97 mg, 55.04 μmol, 43% yield, 98.40% purity, FA) as a yellow solid. ¹HNMR (400 MHz, CD₃OD): δ 7.81 (s, 1H), 7.73 (d, J=8.3 Hz, 1H), 7.42-7.35 (m, 1H), 7.32-7.22 (m, 3H), 7.21-7.15 (m, 1H), 7.08-7.01 (m, 2H), 6.84-6.74 (m, 2H), 6.31-6.22 (m, 1H), 5.82-5.75 (m, 1H), 5.16-5.07 (m, 1H), 4.31-4.19 (m, 2H), 4.02-3.73 (m, 8H), 3.53-3.42 (m, 2H), 3.23-3.11 (m, 2H), 2.94-2.82 (m, 1H), 2.80-2.70 (m, 1H), 2.50-2.35 (m, 1H), 2.19-2.10 (m, 1H), 1.71-1.55 (m, 4H), 1.39 (m, 8H). LC-MS: MS (ES⁺): RT=2.883 min, m/z=847.3 [M+H⁺]; LCMS method: LC-MS METHOD 01.M.

### Example 7—Preparation of Compound I-7

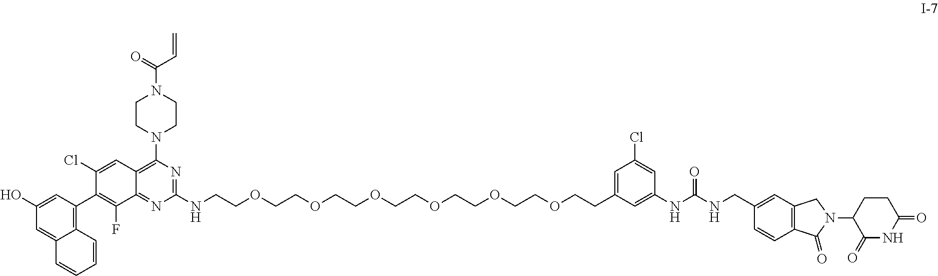 [SMILES: C=CC(=O)N1CCN(c2nc(NCCOCCOCCOCCOCCOCCOCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

The title compound was prepared according to the following procedures.

### 1. Preparation of Compound 2

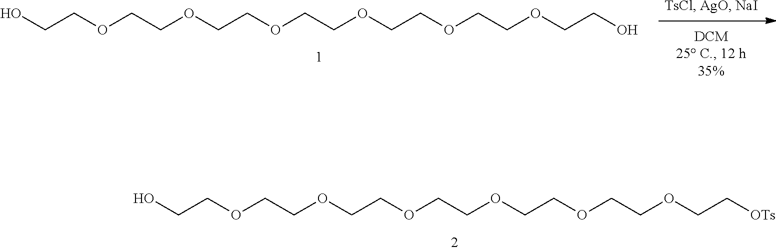

To a solution of 2-[2-[2-[2-[2-[2-(2-hydroxyethoxy)ethoxy]ethoxy]ethoxy]ethoxy]ethoxy]ethanol (10 g, 30 mmol, 1.0 equiv in DCM (100 mL) was added Ag₂O (10.65 g, 45.96 mmol, 1.5 equiv), NaI (5.05 g, 33.7 mmol, 1.1 equiv), 4-methylbenzenesulfonyl chloride (5.84 g, 30.6 mmol, 1.0 equiv) at 0° C. The reaction mixture was stirred at 25° C. for 2 h. The reaction mixture was filtered to remove Ag₂O and NaI. The filtrate was diluted with water (150 mL), extracted with DCM (2×100 mL), dried over Na₂SO₄, filtered and concentrated to afford a crude product. The crude product was purified by column chromatography (SiO₂, PE:EA=5:1 1:1 to EA:MeOH=10:1 to 5:1) to give a desired product 2-[2-[2-[2-[2-[2-(2-hydroxyethoxy)ethoxy]ethoxy]ethoxy]ethoxy]ethoxy]ethyl 4-methylbenzenesulfonate (5.2 g, 10 mmol, 35% yield) as a colorless oil. ¹H NMR (400 MHz, CDCl₃) δ (ppm) 7.72 (d, J=8.4 Hz, 2H), 7.28 (d, J=8.0 Hz, 2H), 4.14-3.98 (m, 2H), 3.67-3.41 (m, 26H), 3.01 (br s, 1H), 2.38 (s, 3H). LC-MS: MS (ES⁺): m/z=481.3 [M+H⁺].

### 2. Preparation of Compound 3

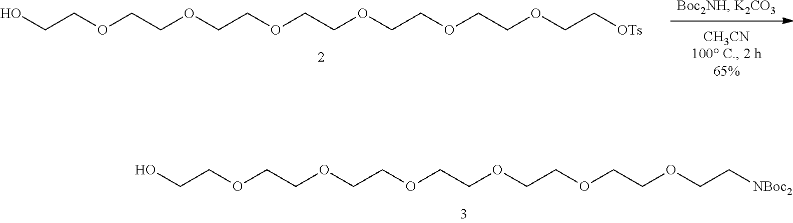

To a solution of 2-[2-[2-[2-[2-[2-(2-hydroxyethoxy)ethoxy]ethoxy]ethoxy]ethoxy]ethoxy]ethyl 4-methylbenzenesulfonate (5.2 g, 10.0 mmol, 1.0 equiv) in CH₃CN (100 mL) was added tert-butyl N-tert-butoxycarbonylcarbamate (3.5 g, 16.2 mmol, 1.5 equiv), K₂CO₃ (4.49 g, 32.4 mmol, 3.0 equiv), it was stirred at 100° C. for 12 h. The reaction mixture was filtered to remove K₂CO₃. The filtrate was diluted with water (50 mL), extracted with EtOAc (3×100 mL), dried over Na₂SO₄, filtered and concentrated to afford a crude product. The crude product was purified by column chromatography (SiO₂, PE:EA=5:1 to 1:1 to EA:MeOH=10:1) to give the desired product tert-butyl N-tert-butoxycarbonyl-N-[2-[2-[2-[2-[2-[2-(2-hydroxyethoxy)ethoxy]ethoxy]ethoxy]ethoxy]ethoxy]ethyl]carbamate (3.71 g, 7.06 mmol, 65% yield) as a colorless oil. ¹H NMR (400 MHz, CDCl₃) δ (ppm) 3.81-3.64 (m, 20H), 3.63-3.58 (m, 8H), 1.50 (s, 18H). LC-MS: MS (ES⁺): m/z=326.1 [M+H⁺].

### 3. Preparation of Compound 4

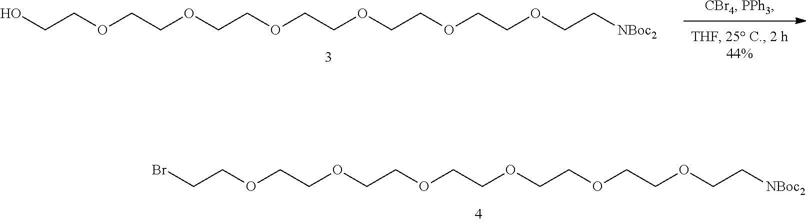

To a solution of tert-butyl N-tert-butoxycarbonyl-N-[2-[2-[2-[2-[2-[2-(2-hydroxyethoxy)ethoxy]ethoxy]ethoxy]ethoxy]ethoxy]ethyl]carbamate (1.8 g, 3.4 mmol, 1.0 equiv) in THF (30 mL) was added PPh₃ (1.80 g, 6.85 mmol, 2.0 equiv) CBr₄ (2.27 g, 6.85 mmol, 2.0 equiv) at 0° C. The mixture was stirred at 25° C. for 2 h. The reaction mixture was concentrated to afford a crude product. The crude product was purified by column chromatography (SiO₂, PE:EA=2:1 to 1:1) and prep-HPLC(column: Waters Xbridge 150*25 mm*5 μm; mobile phase: [water (10 mM NH₄HCO₃)-ACN]; B %: 40%-70%, 10 min) to give the desired product tert-butyl N-[2-[2-[2-[2-[2-[2-(2-bromoethoxy)ethoxy]ethoxy]ethoxy]ethoxy]ethoxy]ethyl]-N-tert-butoxycarbonyl-carbamate (0.89 g, 1.51 mmol, 44% yield) as a colorless oil. ¹H NMR (400 MHz, CDCl₃) δ (ppm) 3.82 (td, J=6.2, 10.4 Hz, 4H), 3.71-3.59 (m, 22H), 3.50 (t, J=6.4 Hz, 2H), 1.52 (s, 18H). LC-MS: MS (ES⁺): m/z=605.2[M+18].

### 4. Preparation of Compound 6

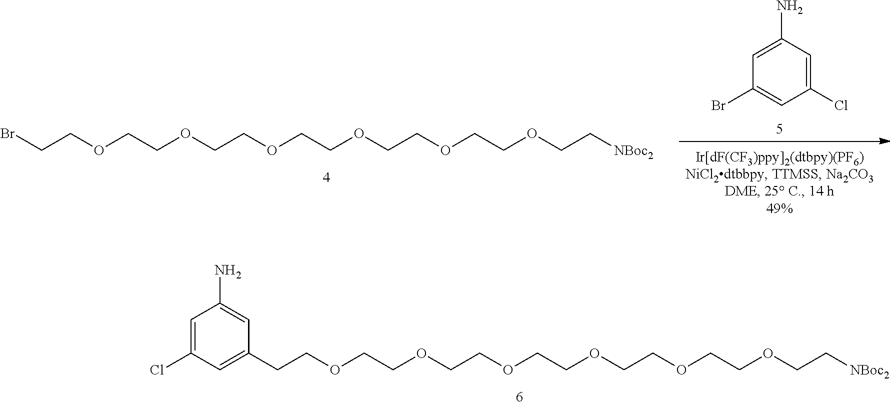

To a 15 mL vial equipped with a stir bar was added tert-butyl N-[2-[2-[2-[2-[2-[2-(2-bromoethoxy)ethoxy]ethoxy]ethoxy]ethoxy]ethoxy]ethyl]-N-tert-butoxycarbonyl-carbamate (1.73 g, 2.94 mmol, 1.3 equiv) 3-bromo-5-chloro-aniline (466 mg, 2.26 mmol, 1.0 equiv), Ir[dF(CF₃)ppy]₂(dtbpy)(PF₆) (25.4 mg, 22.61 μmol, 0.01 equiv), NiCl₂.dtbbpy (45.0 mg, 113.0 μmol, 0.05 equiv), TTMSS (562 mg, 2.26 mmol, 697 μL, 1.0 equiv), Na₂CO₃ (479 mg, 4.52 mmol, 2.0 equiv) and DME (40 mL). The vial was sealed and placed under nitrogen. The reaction was stirred and irradiated with a 34 W blue LED lamp (7 cm away), with cooling fan to keep the reaction temperature at 25° C. for 14 h. The reaction mixture was filtered and concentrated under reduced pressure to give a residue. The residue was purified by column chromatography (SiO₂, Petroleum ether/Ethyl acetate=1/1 to EA/MEOH=10/1). The residue was purified by prep-HPLC (column: Waters Xbridge C18 150*50 mm*10 μm; mobile phase: [water (10 mM NH₄HCO₃)-ACN]; B %: 39%-69%, 8 min) to give the desired product tert-butyl N-[2-[2-[2-[2-[2-[2-[2-(3-amino-5-chloro-phenyl)ethoxy]ethoxy]-ethoxy]-ethoxy]ethoxy]ethoxy]ethyl]-N-tert-butoxycarbonyl-carbamate (706 mg, 1.11 mmol, 49% yield) as a colorless oil. ¹H NMR (400 MHz, CDCl₃) S (ppm)=7.71-7.35 (m, 1H), 7.15-6.97 (m, 1H), 6.93-6.68 (m, 1H), 3.75-3.46 (m, 26H), 2.86-2.61 (m, 2H), 1.46-1.38 (m, 18H). LC-MS: MS (ES⁺): m/z=605.2[M+18].

### 5. Preparation of Compound 8

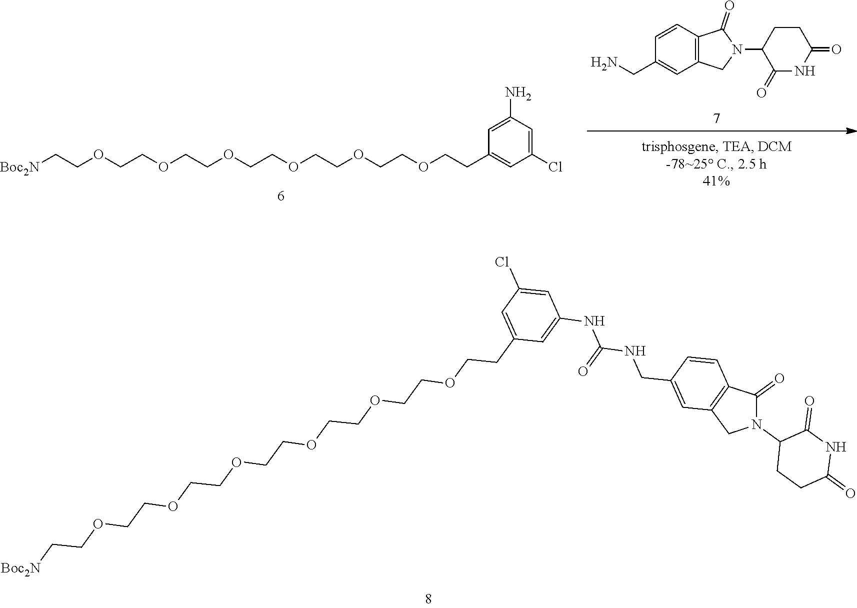

To a solution of triphosgene (170 mg, 572 μmol, 1.46 equiv) in DCM (3 mL) at −78° C. was added TEA (318 mg, 3.15 mmol, 438 μL, 8.0 equiv), then tert-butyl N-[2-[2-[2-[2-[2-[2-[2-(3-amino-5-chloro-phenyl)ethoxy]ethoxy]ethoxy]ethoxy]ethoxy]ethoxy]ethyl]-N-tert-butoxycarbonyl-carbamate (250 mg, 393 μmol, 1.0 equiv) in DCM (2 mL) was added and the mixture was stirred for 0.5 h. 3-[5-(aminomethyl)-1-oxo-isoindolin-2-yl]piperidine-2,6-dione (146.29 mg, 472.30 μmol, 1.2 equiv, HCl) was added, then the mixture was allowed to warm to 25° C. and stirred for 2 h. The reaction mixture was poured into saturated NaHCO₃ (20 mL), extracted with DCM (2×20 mL), dried over Na₂SO₄, filtered and concentrated to afford a crude product. The crude product was purified by prep-TLC (SiO₂ DCM:MeOH=10:1) to give a desired product tert-butyl N-tert-butoxycarbonyl-N-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethyl]carbamat e (151 mg, 161 μmol, 41% yield) as a colorless oil. LC-MS: MS (ES⁺): RT=0.931 min, m/z=951.2[M+H₃O⁺].

### 6. Preparation of Compound 9

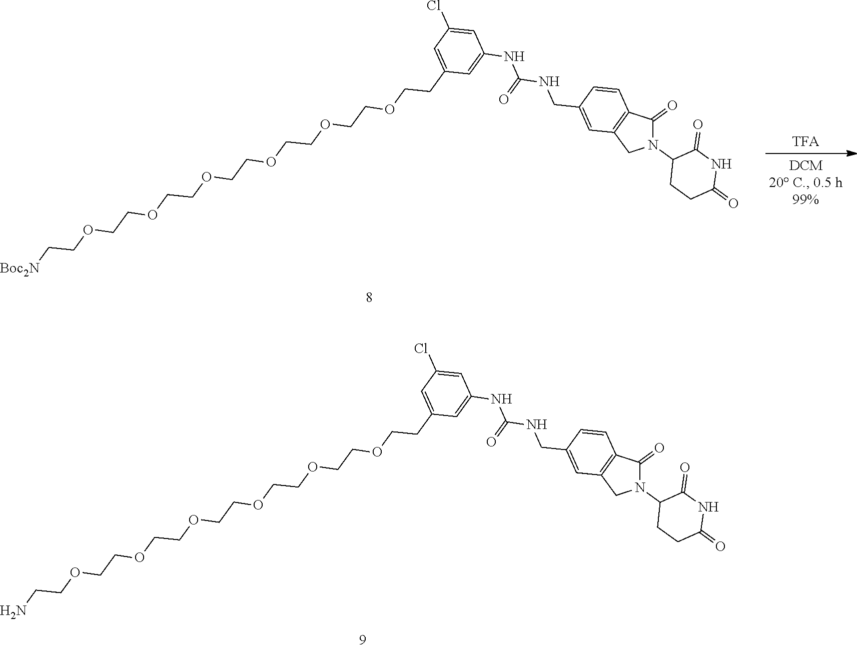

To a solution of tert-butyl N-tert-butoxycarbonyl-N-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]-ethoxy]ethoxy]ethyl]carbamate (42 mg, 44 μmol, 1.0 equiv) in DCM (5 mL) was added TFA (1 mL). The reaction mixture was stirred at 20° C. for 0.5 h. The mixture was concentrated to afford product 1-[3-[2-[2-[2-[2-[2-[2-(2-aminoethoxy)ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (38.13 mg, 44.95 μmol, 100% yield, TFA salt) as a black brown oil. LC-MS: MS (ES⁺): m/z=734.2[M+H⁺].

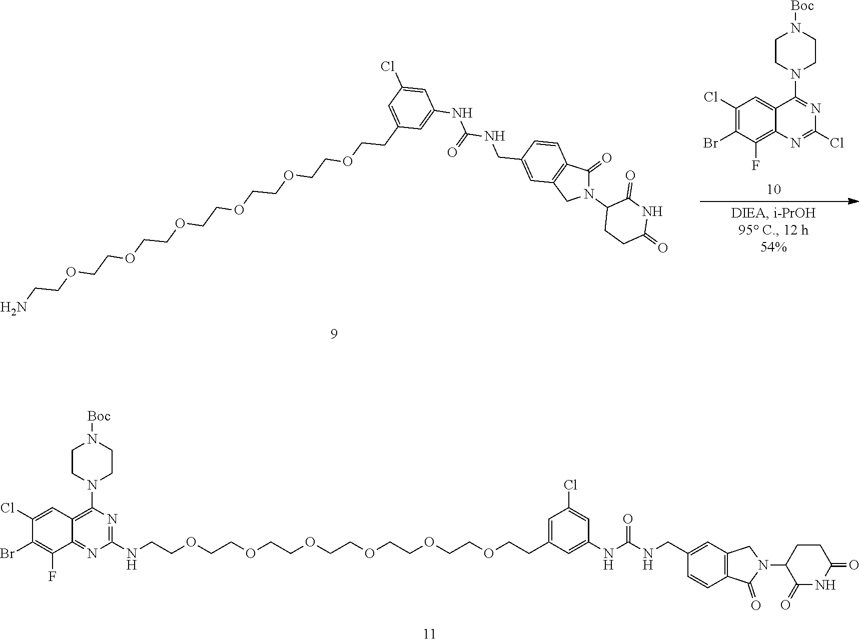 [SMILES: NCCOCCOCCOCCOCCOCCOCCc1cc(Cl)cc(NC(=O)NCc2ccc3c(c2)CN(C2CCC(=O)NC2=O)C3=O)c1 ; CC(C)(C)OC(=O)N1CCN(c2nc(Cl)nc3c(F)c(Br)c(Cl)cc23)CC1 ; CC(C)(C)OC(=O)N1CCN(c2nc(NCCOCCOCCOCCOCCOCCOCCc3cc(Cl)cc(NC(=O)NCc4ccc5c(c4)CN(C4CCC(=O)NC4=O)C5=O)c3)nc3c(F)c(Br)c(Cl)cc23)CC1]

To a mixture of 1-[3-[2-[2-[2-[2-[2-[2-(2-aminoethoxy)ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (145 mg, 170 μmol, 1.0 equiv, TFA) and tert-butyl 4-(7-bromo-2,6-dichloro-8-fluoro-quinazolin-4-yl)piperazine-1-carboxylate (82.08 mg, 170.9 μmol, 1.0 equiv) in i-PrOH (3 mL) was added DIEA (110.46 mg, 854.69 μmol, 148.87 μL, 5.0 equiv). The mixture was stirred at 90° C. for 12 h. The mixture was concentrated to give a residue. The residue was purified by prep-TLC (Dichloromethane:Methanol=10:1) to give the desired product tert-butyl 4-[7-bromo-6-chloro-2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethylamino]-8-fluoro-quinazolin-4-yl]piperazine-1-carboxylate (110 mg, 93.3 μmol, 54% yield) as a yellow solid. LC-MS: MS (ES⁺): m/z=1178.1[M+H⁺].

### 8. Preparation of Compound 13

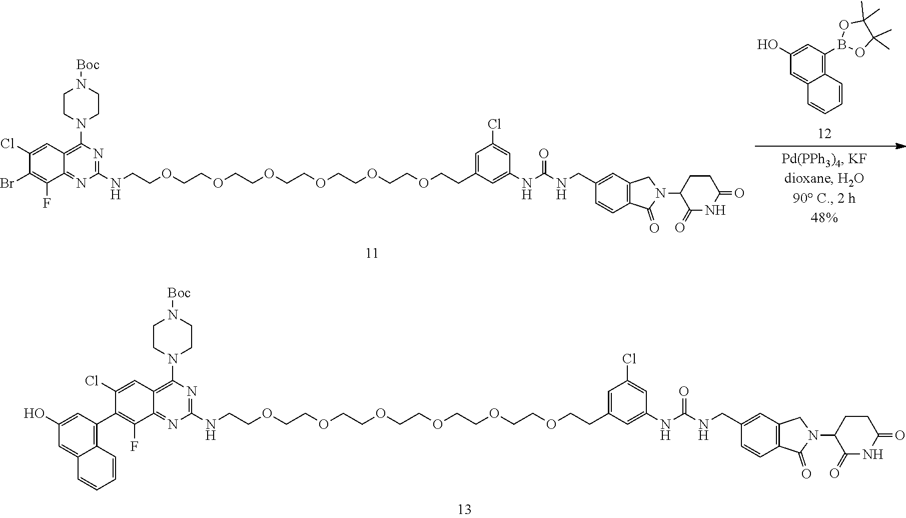 [SMILES: CC(C)(C)OC(=O)N1CCN(c2nc(NCCOCCOCCOCCOCCOCCOCCc3cc(Cl)cc(NC(=O)NCc4ccc5c(c4)CN(C4CCC(=O)NC4=O)C5=O)c3)nc3c(F)c(Br)c(Cl)cc23)CC1 ; CC1(C)OB(c2cc(O)cc3ccccc23)OC1(C)C ; CC(C)(C)OC(=O)N1CCN(c2nc(NCCOCCOCCOCCOCCOCCOCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

To a mixture of tert-butyl 4-[7-bromo-6-chloro-2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethylamino]-8-fluoro-quinazolin-4-yl]piperazine-1-carboxylate (90 mg, 76 μmol, 1.0 equiv), Pd(PPh₃)₄ (44.15 mg, 38.20 μmol, 0.5 equiv) and KF (11.10 mg, 191.0 umol, 4.470 μL, 2.5 equiv) in dioxane (10 mL) and H₂O (2.5 mL) was added 4-(4,4,5,5-tetramethyl-1,3,2-dioxaborolan-2-yl)naphthalen-2-ol (30.96 mg, 114.6 μmol, 1.5 equiv). After addition, the mixture was stirred at 90° C. for 2 h. The mixture was diluted with water (20 mL). The solution was extracted with EtOAc (2×50 mL). The combined organic layers were dried, filtered and concentrated to give a residue. The residue was purified by prep-TLC (SiO₂, Dichloromethane:Methanol=10:1) to give the desired product tert-butyl-4-[6-chloro-2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (55 mg, 37 μmol, 48% yield, 84% purity) as a yellow solid. LC-MS: MS (ES⁺): m/z=1241.9[M+H⁺].

### 9. Preparation of Compound 14

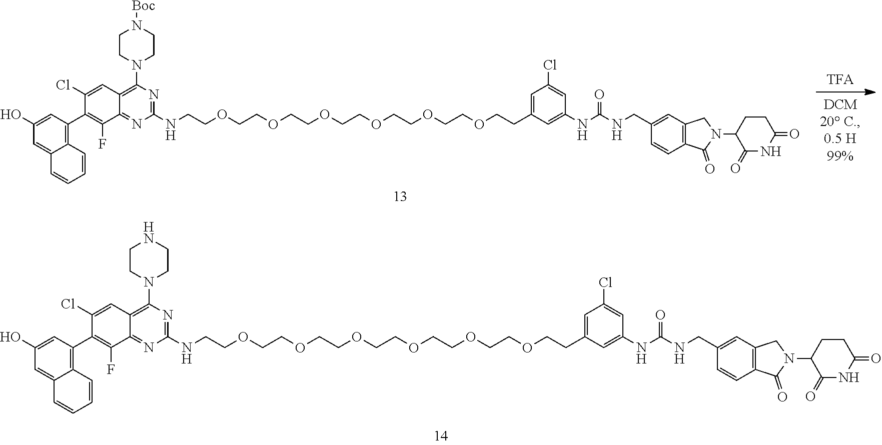 [SMILES: CC(C)(C)OC(=O)N1CCN(c2nc(NCCOCCOCCOCCOCCOCCOCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1 ; O=C1CCC(N2Cc3cc(CNC(=O)Nc4cc(Cl)cc(CCOCCOCCOCCOCCOCCOCCNc5nc(N6CCNCC6)c6cc(Cl)c(-c7cc(O)cc8ccccc78)c(F)c6n5)c4)ccc3C2=O)C(=O)N1]

To a solution of tert-butyl 4-[6-chloro-2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (55 mg, 44 μmol, 1.0 equiv) in DCM (2 mL) was added TFA (1.01 g, 8.86 mmol, 656 μL, 200.0 equiv), it was stirred at 20° C. for 15 mins. The reaction mixture was concentrated to afford a product 1-[3-chloro-5-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (55 mg, 43 μmol, 98% yield, TFA salt) as a colorless oil. LC-MS: MS (ES⁺): m/z=1140.5[M+H⁺].

### 10. Preparation of I-7

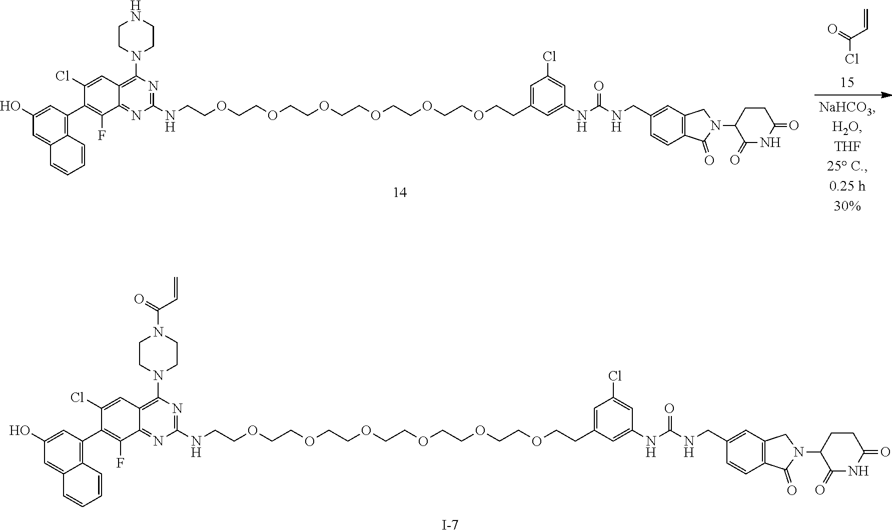

To a solution of 1-[3-chloro-5-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (55 mg, 43 μmol, 1.0 equiv, TFA salt) in THE (4 mL) was added NaHCO₃ (184 mg, 2.19 mmol, 85.2 μL, 50.0 equiv) in H₂O (4 mL). Then prop-2-enoyl chloride (3.97 mg, 43.8 μmol, 3.57 μL, 1.0 equiv) in THE (1 mL) was added at 0° C. The mixture was stirred at 0° C. for 15 mins. The reaction mixture was diluted with brine (10 mL) and extracted with EtOAc (2×30 mL). The combined organic layers were dried over anhydrous sodium sulfate, filtered and concentrated to afford a crude product. The crude product was purified by prep-HPLC (column: Phenomenex Gemini-NX C18 75*30 mm*3 μm; mobile phase: [water (0.225% FA)-ACN]; B %: 22%-52%, 7 min) to give the desired product 1-[3-chloro-5-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]amino]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (16.19 mg, 13.55 μmol, 30% yield) as a white solid. ¹H NMR (400 MHz, CD₃OD) δ (ppm) 7.71 (s, 1H), 7.66-7.60 (m, 2H), 7.40 (s, 1H), 7.37-7.26 (m, 3H), 7.18-7.12 (m, 2H), 7.11-7.06 (m, 1H), 7.00-6.93 (m, 2H), 6.73 (d, J=1.2 Hz, 1H), 6.71-6.65 (m, 1H), 6.17 (dd, J=1.8, 16.8 Hz, 1H), 5.73-5.66 (m, 1H), 5.01 (dd, J=4.4, 13.6 Hz, 1H), 4.38 (s, 2H), 4.29 (d, J=3.4 Hz, 2H), 3.77 (m, 7H), 3.58-3.41 (m, 28H), 2.84-2.70 (m, 1H), 2.71-2.70 (m, 1H), 2.68-2.60 (m, 3H), 2.31 (dq, J=4.4, 13.2 Hz, 1H), 2.01 (dtd, J=2.6, 5.2, 12.4 Hz, 1H). LC-MS: MS (ES⁺): RT=0.608 min, m/z=1194.5[M+H⁺].

### Example 8—Preparation of Compound I-8

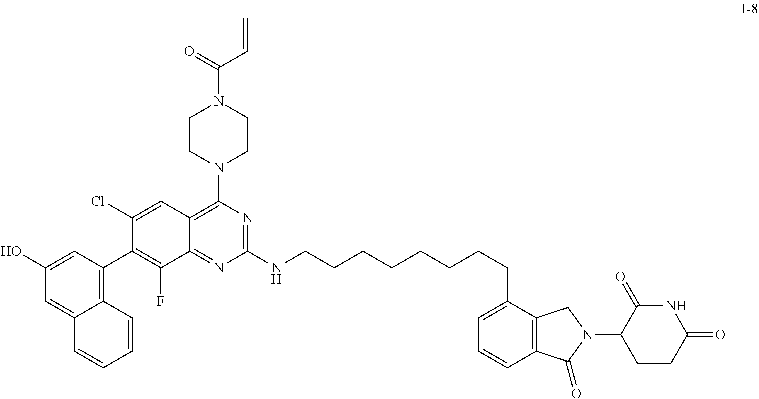 [SMILES: C=CC(=O)N1CCN(c2nc(NCCCCCCCCc3cccc4c3CN(C3CCC(=O)NC3=O)C4=O)nc3c(F)c(-c4cc(O)cc5ccccc45)c(Cl)cc23)CC1]

The title compound was prepared according to the following procedures.

### 1. Preparation of Compound 2

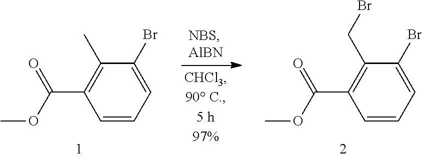

A mixture of compound 1 (5.00 g, 21.83 mmol, 1.0 equiv), NBS (4.45 g, 25.01 mmol, 1.15 equiv), AIBN (358.42 mg, 2.18 mmol, 0.1 equiv) in CHCl₃ (50 mL) was degassed and purged with N₂ for 3 times, and then the mixture was stirred at 90° C. for 5 h under N₂ atmosphere. The reaction mixture was concentrated under reduced pressure to give a residue. The residue was purified by column chromatography (SiO₂, Petroleum ether/Ethyl acetate=1/0 to 10/1). Compound 2 (6.50 g, 21.11 mmol, 97% yield) was obtained as a yellow oil. ¹HNMR (400 MHz, CDCl₃): δ 7.81 (d, J=7.8 Hz, 1H), 7.69 (d, J=7.9 Hz, 1H), 7.23-7.12 (m, 1H), 5.06 (s, 2H), 3.88 (s, 3H).

### 2. Preparation of Compound 4

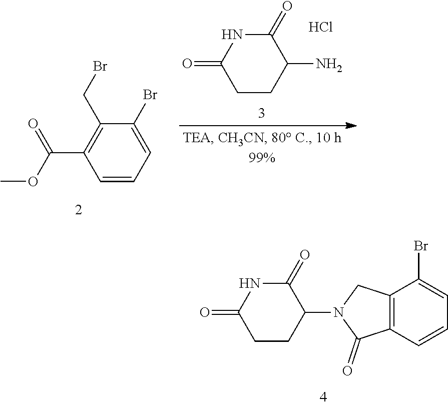 [SMILES: COC(=O)c1cccc(Br)c1CBr ; NC1CCC(=O)NC1=O ; Cl ; O=C1CCC(N2Cc3c(Br)cccc3C2=O)C(=O)N1]

To a solution of compound 2 (6.11 g, 19.84 mmol, 1.0 equiv) in CH₃CN (50 mL) was added TEA (2.69 g, 26.57 mmol, 3.70 mL, 1.34 equiv) and compound 3 (4.25 g, 25.79 mmol, 1.3 equiv). The mixture was stirred at 80° C. for 10 h. The reaction mixture was filtered and the filter cake was concentrated under reduced pressure to give a residue. The purple solid was used for next step without purification. Compound 4 (6.40 g, 19.81 mmol, 99% yield) was obtained as a purple solid. ¹HNMR (400 MHz, DMSO-d₆): δ 11.02 (s, 1H), 7.87 (d, J=7.8 Hz, 1H), 7.77 (d, J=7.5 Hz, 1H), 7.55-7.48 (m, 1H), 5.21-5.09 (m, 1H), 4.46-4.38 (m, 1H), 4.26 (m, 1H), 4.21-4.15 (m, 1H), 2.97-2.86 (m, 1H), 2.78-2.66 (m, 1H), 2.49-2.41 (m, 1H). LC-MS: MS (ES⁺): m/z=322.9 [M+H⁺].

### 3. Preparation of Compound 6

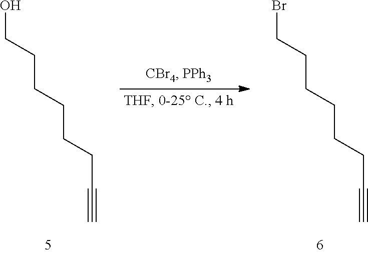

To a solution of PPh₃ (16.63 g, 63.39 mmol, 2.0 eq) in THF (40 mL) was added CBr₄ (21.02 g, 63.39 mmol, 2.0 eq) at 0° C. Then compound 5 (4.00 g, 31.70 mmol, 1.0 eq) was added at 0° C. The mixture was stirred at 25° C. for 4 h. The reaction mixture was concentrated under reduced pressure. The residue was purified by column chromatography (SiO₂, Petroleum ether/Ethyl acetate=1/0 to 10/1). Compound 6 (7.66 g, crude) was obtained as a colorless oil. ¹HNMR (400 MHz, DMSO-d₆): δ 3.55-3.49 (m, 2H), 2.18-2.11 (m, 2H), 1.96-1.90 (m, 1H), 1.83-1.73 (m, 2H), 1.49-1.32 (m, 6H).

### 4. Preparation of Compound 8

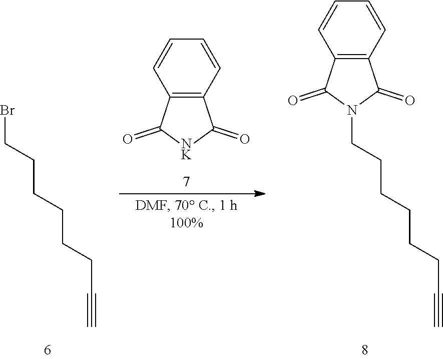

To a solution of compound 6 (7.66 g, 40.51 mmol, 1.0 equiv) in DMF (50 mL) was added compound 7 (11.25 g, 60.76 mmol, 1.5 equiv). The mixture was stirred at 70° C. for 1 h. The reaction mixture was concentrated under reduced pressure. Compound 8 (10.34 g, crude) was obtained as a yellow solid. The yellow solid was used for next step without purification. LC-MS: MS (ES⁺): m/z=256.1 [M+H⁺]

### 5. Preparation of Compound 9

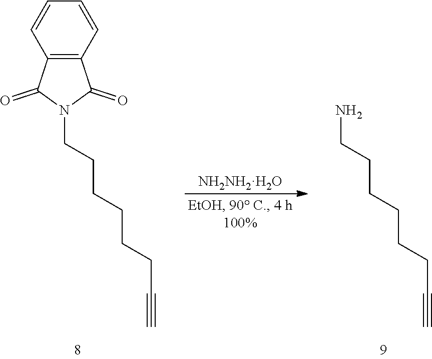

To a solution of compound 8 (10.34 g, crude) in EtOH (100 mL) was added NH₂NH₂·H₂O (10.79 g, 215.48 mmol, 10.47 mL, 5.3 eq). The mixture was stirred at 90° C. for 4 h. The reaction mixture was filtered and the filtrate was concentrated under reduced pressure to give a residue. Compound 9 (5.07 g, 40.49 mmol, 100.00% yield) was obtained as a yellow solid. The residue was used for next step without purification.

### 6. Preparation of Compound 10

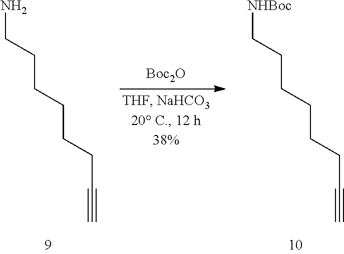

To a solution of compound 9 (5.07 g, 40.49 mmol, 1.0 equiv) in THF (50 mL) and NaHCO₃ (50 mL) was added Boc₂O (35.35 g, 161.97 mmol, 37.21 mL, 4.0 equiv). The mixture was stirred at 20° C. for 12 h. The reaction mixture was diluted with H₂O (50 mL) and extracted with ethyl acetate (EA) (2×80 mL). The combined organic layers were dried over Na₂SO₄, filtered and concentrated under reduced pressure to give a residue. The residue was purified by column chromatography (SiO₂, Petroleum ether/Ethyl acetate=3/1 to 0/1). Compound 10 (3.50 g, 15.53 mmol, 38% yield) was obtained as a colorless oil. ¹HNMR (400 MHz, CDCl₃): δ 4.51 (s, 1H), 3.20-3.03 (m, 2H), 2.24-2.15 (m, 2H), 1.98-1.91 (m, 1H), 1.60 (s, 2H), 1.56-1.45 (m, 9H), 1.44-1.25 (m, 5H).

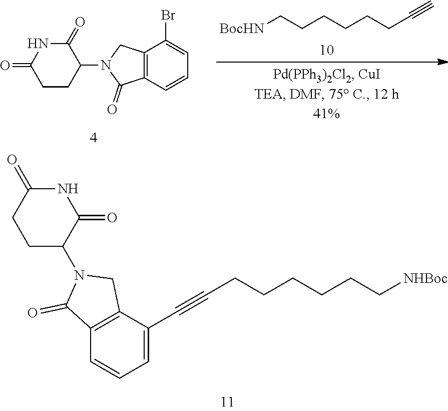 [SMILES: O=C1CCC(N2Cc3c(Br)cccc3C2=O)C(=O)N1 ; C#CCCCCCCNC(=O)OC(C)(C)C ; CC(C)(C)OC(=O)NCCCCCCC#Cc1cccc2c1CN(C1CCC(=O)NC1=O)C2=O]

A mixture of compound 10 (697 mg, 3.09 mmol, 1.0 equiv), compound 4 (1.00 g, 3.09 mmol, 1.0 equiv), Pd(PPh₃)₂Cl₂ (217 mg, 309.46 μmol, 0.1 equiv), CuI (118 mg, 618.92 μmol, 0.2 equiv) and TEA (1.25 g, 12.38 mmol, 1.72 mL, 4.0 equiv) in DMF (10 mL) was degassed and purged with N₂ for 3 times, and then the mixture was stirred at 75° C. for 12 h under N₂ atmosphere. The reaction mixture was diluted with H₂O (50 mL) and extracted with EA (2×50 mL). The combined organic layers were dried over Na₂SO₄, filtered and concentrated under reduced pressure to give a residue. The residue was purified by column chromatography (SiO₂, Petroleum ether/Ethyl acetate=1/1 to 0/1). Compound 11 (600 mg, 1.28 mmol, 41% yield) was obtained as a yellow solid. ¹HNMR (400 MHz, DMSO-d₆): δ 10.99 (s, 1H), 7.70 (d, J=7.5 Hz, 1H), 7.63 (d, J=7.3 Hz, 1H), 7.55-7.48 (m, 1H), 6.79-6.72 (m, 1H), 5.20-5.06 (m, 1H), 4.51-4.23 (m, 2H), 2.99-2.82 (m, 3H), 2.69-2.55 (m, 2H), 2.47-2.38 (m, 2H), 2.07-1.97 (m, 1H), 1.62-1.50 (m, 2H), 1.48-1.17 (m, 15H). LC-MS: MS (ES⁺): m/z=490.4 [M+Na⁺].

### 8. Preparation of Compound 12

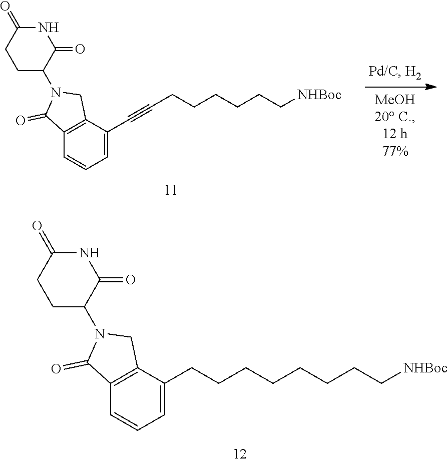 [SMILES: CC(C)(C)OC(=O)NCCCCCCC#Cc1cccc2c1CN(C1CCC(=O)NC1=O)C2=O ; CC(C)(C)OC(=O)NCCCCCCCCc1cccc2c1CN(C1CCC(=O)NC1=O)C2=O]

To a solution of compound 11 (600 mg, 1.28 mmol, 1.0 equiv) in MeOH (1 mL) was added Pd/C (60 mg, 10% purity) under N₂ atmosphere. The suspension was degassed and purged with H₂ for 3 times. The mixture was stirred under H₂ (15 Psi) at 20° C. for 12 h. The reaction mixture was filtered and concentrated under reduced pressure to give a residue. Compound 12 (467 mg, 990 μmol, 77% yield) was obtained as a white solid. The white solid was used for next step without purification. LC-MS: MS (ES⁺): m/z=494.2 [M+Na⁺].

### 9. Preparation of Compound 13

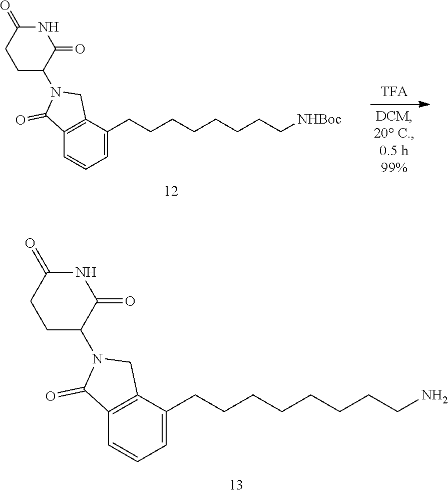 [SMILES: CC(C)(C)OC(=O)NCCCCCCCCc1cccc2c1CN(C1CCC(=O)NC1=O)C2=O ; NCCCCCCCCc1cccc2c1CN(C1CCC(=O)NC1=O)C2=O]

To a solution of compound 12 (467 mg, 990 μmol, 1.0 equiv) in CH₂Cl₂ (12 mL) was added TFA (4 mL). The mixture was stirred at 20° C. for 0.5 h. The reaction mixture was concentrated under reduced pressure. Compound 13 (480 mg, 988 μmol, 99% yield, TFA) was obtained as a yellow oil. The yellow oil was used for next step without purification. LC-MS: MS (ES⁺): m/z=372.0 [M+H⁺].

### 10. Preparation of Compound 15

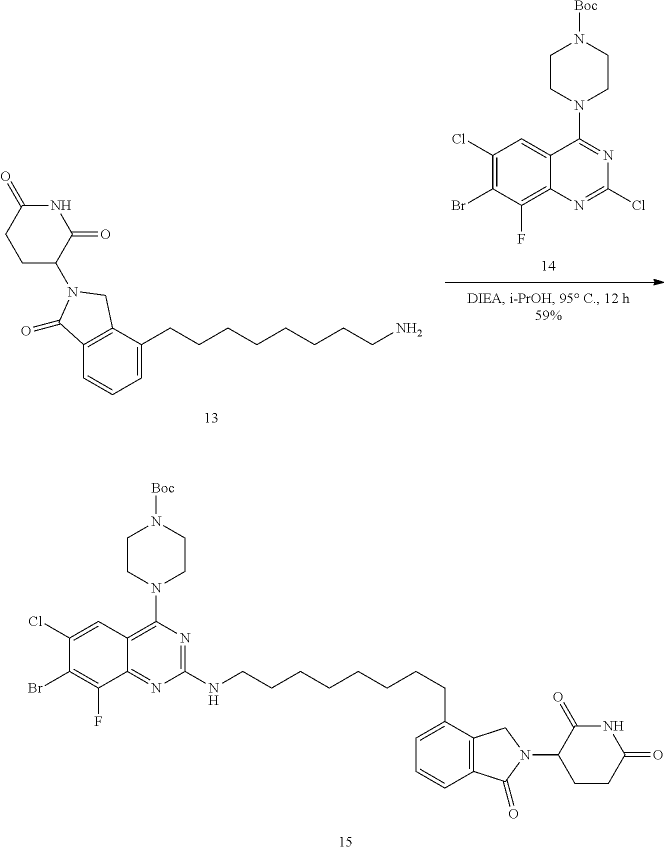 [SMILES: NCCCCCCCCc1cccc2c1CN(C1CCC(=O)NC1=O)C2=O ; CC(C)(C)OC(=O)N1CCN(c2nc(Cl)nc3c(F)c(Br)c(Cl)cc23)CC1 ; CC(C)(C)OC(=O)N1CCN(c2nc(NCCCCCCCCc3cccc4c3CN(C3CCC(=O)NC3=O)C4=O)nc3c(F)c(Br)c(Cl)cc23)CC1]

To a solution of compound 13 (480 mg, 988 μmol, 1.0 equiv, TFA) and DIEA (383 mg, 2.97 mmol, 516 μL, 3.0 equiv) in i-PrOH (14 mL) was added compound 14 (475 mg, 988 μmol, 1.0 equiv). The mixture was stirred at 95° C. for 12 h. The reaction mixture was concentrated under reduced pressure. The residue was purified by column chromatography (SiO₂, Petroleum ether/Ethyl acetate=5/1 to 1/2). Compound 15 (480 mg, 589 μmol, 59% yield) was obtained as a yellow solid. LC-MS: MS (ES⁺): m/z=816.0 [M+H⁺].

### 11. Preparation of Compound 17

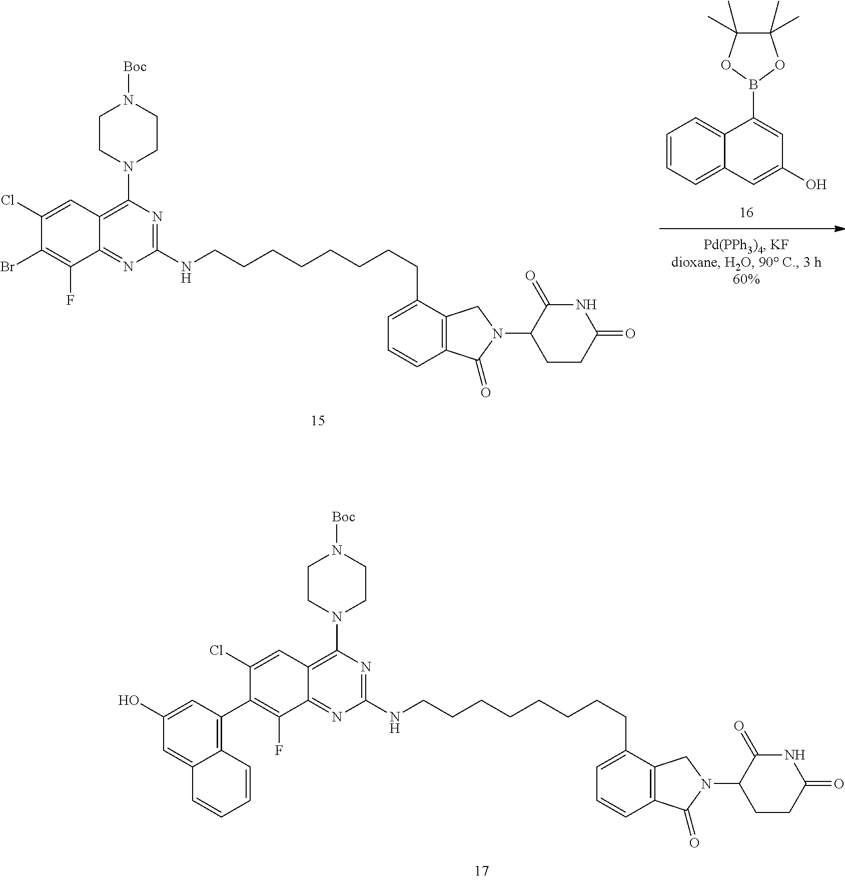 [SMILES: CC(C)(C)OC(=O)N1CCN(c2nc(NCCCCCCCCc3cccc4c3CN(C3CCC(=O)NC3=O)C4=O)nc3c(F)c(Br)c(Cl)cc23)CC1 ; CC1(C)OB(c2cc(O)cc3ccccc23)OC1(C)C ; CC(C)(C)OC(=O)N1CCN(c2nc(NCCCCCCCCc3cccc4c3CN(C3CCC(=O)NC3=O)C4=O)nc3c(F)c(-c4cc(O)cc5ccccc45)c(Cl)cc23)CC1]

A mixture of compound 15 (200 mg, 245 μmol, 1.0 equiv), compound 16 (99 mg, 368 μmol, 1.5 equiv), Pd(PPh₃)₄ (142 mg, 123 μmol, 0.5 equiv), KF (36 mg, 613 μmol, 14.4 μL, 2.5 equiv) in dioxane (20 mL) and H₂O (4 mL) was degassed and purged with N₂ for 3 times, and then the mixture was stirred at 90° C. for 3 h under N₂ atmosphere. The reaction mixture was diluted with brine (40 mL) and extracted with EA (2×50 mL). The combined organic layers were washed with brine (40 mL), dried over Na₂SO₄, filtered and concentrated under reduced pressure to give a residue. The residue was purified by prep-TLC (SiO₂, petroleum ether (PE):EA=1:2). Compound 17 (130 mg, 148 μmol, 60% yield) was obtained as a yellow solid. LC-MS: MS (ES⁺): m/z=878.3 [M+H⁺].

### 12. Preparation of Compound 18

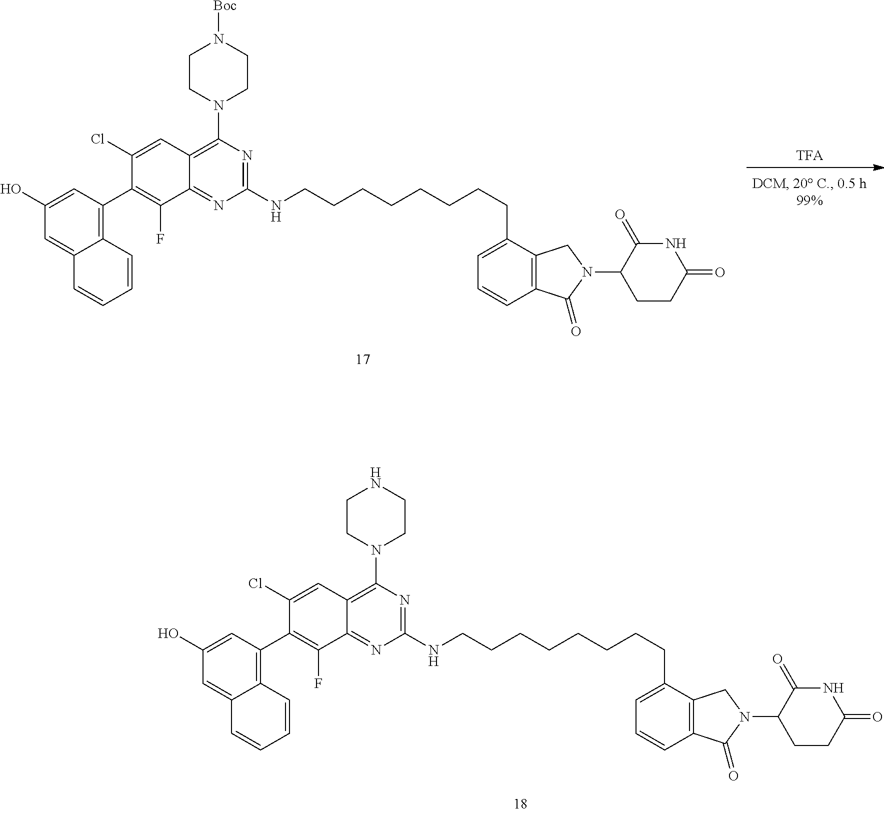 [SMILES: CC(C)(C)OC(=O)N1CCN(c2nc(NCCCCCCCCc3cccc4c3CN(C3CCC(=O)NC3=O)C4=O)nc3c(F)c(-c4cc(O)cc5ccccc45)c(Cl)cc23)CC1 ; "TFA" ; O=C1CCC(N2Cc3c(CCCCCCCCNc4nc(N5CCNCC5)c5cc(Cl)c(-c6cc(O)cc7ccccc67)c(F)c5n4)cccc3C2=O)C(=O)N1]

To a solution of compound (130 mg, 148 μmol, 1.0 equiv) in CH₂Cl₂ (2 mL) was added TFA (0.5 mL). The mixture was stirred at 20° C. for 1 h. The reaction mixture was concentrated under reduced pressure. Compound 18 (132 mg, 148 μmol, 99% yield, TFA) was obtained as a yellow oil. The yellow oil was used for next step without purification. LC-MS: MS (ES⁺): m/z=778.6 [M+H⁺].

### 13. Preparation of I-8

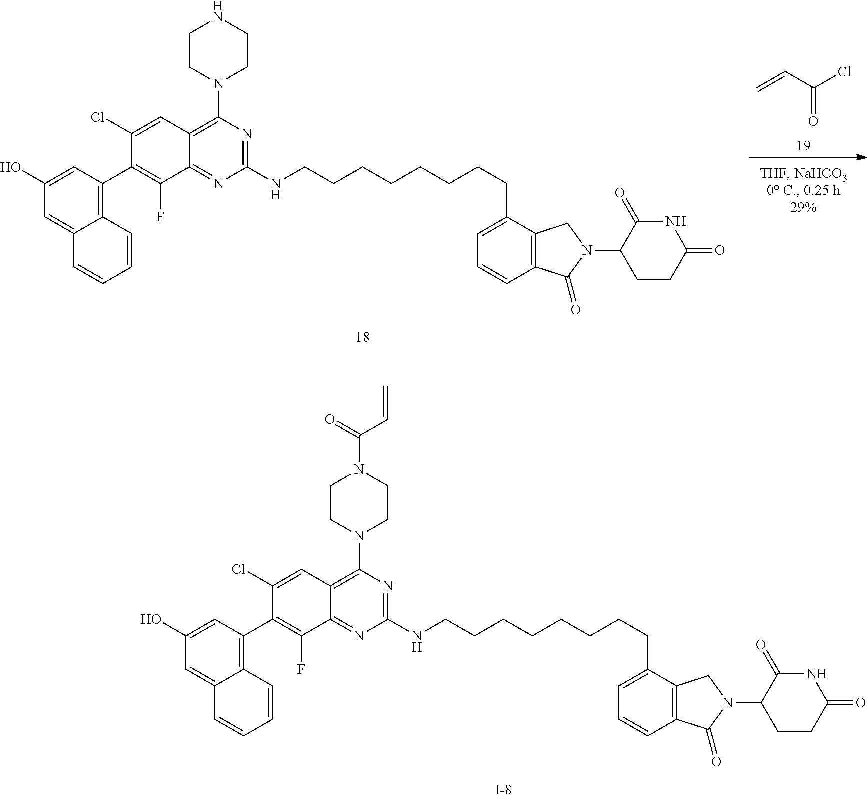

To a solution of compound 18 (132.00 mg, 147.93 μmol, 1.0 eq, TFA) in THF (2.5 mL) and saturated NaHCO₃ (2.5 mL) was added a solution of compound 19 (26.78 mg, 295.85 μmol, 24.12 μL, 2.0 eq) in THF (2.5 mL) at 0° C. The mixture was stirred at 0° C. for 0.25 h. The reaction mixture was diluted with brine (20 mL) and extracted with EA (2×20 mL). The combined organic layers were washed with brine (20 mL), dried over Na₂SO₄, filtered and concentrated under reduced pressure to give a residue. The residue was purified by prep-HPLC (column: Unisil 3-100 C18 Ultra 150*50 mm*3 μm; mobile phase: [water (0.225% FA)-ACN]; B %: 30%-60%, 10 min). Compound 3-(4-(8-((4-(4-acryloylpiperazin-1-yl)-6-chloro-8-fluoro-7-(3-hydroxynaphthalen-1-yl) quinazolin-2-yl)amino)octyl)-1-oxoisoindolin-2-yl)piperidine-2,6-dione (38.63 mg, 42.59 μmol, 29% yield, 96.85% purity, FA) was obtained as a yellow solid. ¹HNMR (400 MHz, CD₃OD): δ 8.36 (s, 1H), 7.82 (s, 1H), 7.73 (m, 1H), 7.65-7.58 (m, 1H), 7.47-7.34 (m, 3H), 7.33-7.22 (m, 2H), 7.21-7.14 (m, 1H), 7.02 (d, J=2.3 Hz, 1H), 6.87-6.76 (m, 1H), 6.31-6.20 (m, 1H), 5.85-5.74 (m, 1H), 5.20-5.06 (m, 1H), 4.55-4.32 (m, 2H), 4.00-3.73 (m, 8H), 3.55-3.39 (m, 2H), 2.94-2.81 (m, 1H), 2.79-2.59 (m, 3H), 2.56-2.37 (m, 1H), 2.19-2.08 (m, 1H), 1.71-1.58 (m, 4H), 1.45-1.29 (m, 8H). LC-MS: MS (ES⁺): RT=2.203 min, m/z=832.3 [M+H⁺]; LCMS method: LC-MS METHOD 25.M

### Example 9—Preparation of Compound I-9

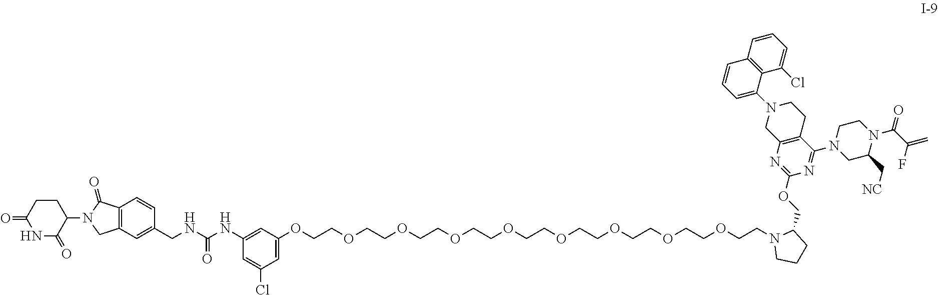 [SMILES: C=C(F)C(=O)N1CCN(c2nc(OC[C@@H]3CCCN3CCOCCOCCOCCOCCOCCOCCOCCOCCOc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N]

The title compound was prepared according to the following procedures.

### 1. Preparation of Compound 2

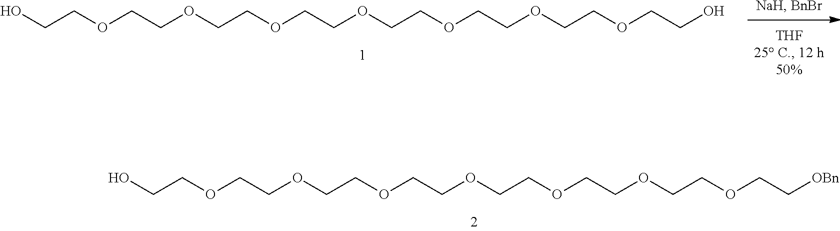

To a solution of 2-[2-[2-[2-[2-[2-[2-(2-hydroxyethoxy)ethoxy]ethoxy]ethoxy]ethoxy]ethoxy]ethoxy]ethanol (10 g, 27 mmol, 1.0 equiv) in THE (60 mL) was slowly added NaH (1.30 g, 32.3 mmol, 60% purity, 1.2 equiv) at 0° C. The solution was stirred at 0° C. for 0.5 h. Then bromomethylbenzene (4.62 g, 27.0 mmol, 3.21 mL, 1.0 equiv) was added at 0° C., and it was stirred at 25° C. for 12 h. The solution was quenched by saturated NH₄Cl (50 mL), extracted with ethyl acetate (2×100 mL), dried, filtered and concentrated to afford a crude product. The crude product was purified by column chromatography (SiO₂, PE:EA=1:1 to 0:1 to EA:MeOH=10:1) to give the desired product 2-[2-[2-[2-[2-[2-[2-(2-benzyloxyethoxy)ethoxy]ethoxy]ethoxy]ethoxy]ethoxy]ethoxy]ethanol (6.30 g, 13.6 mmol, 50% yield) as a colorless oil. ¹H NMR (400 MHz, CDCl₃) δ (ppm) 7.36-7.27 (m, 5H), 4.60-4.54 (m, 2H), 3.76-3.58 (m, 32H), 2.61 (br d, J=1.6 Hz, 1H). LC-MS: MS (ES⁺): m/z=461.2 [M+H⁺].

### 2. Preparation of Compound 3

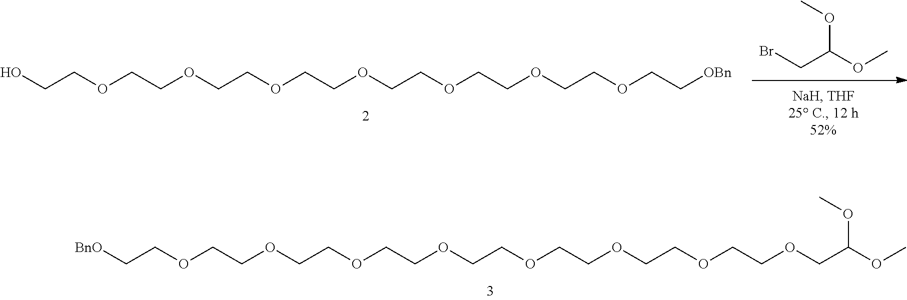

To a reaction mixture of NaH (656.5 mg, 16.41 mmol, 60% purity, 1.2 equiv) in THF (50 mL) was added 2-[2-[2-[2-[2-[2-[2-(2-benzyloxyethoxy)ethoxy]ethoxy]ethoxy]ethoxy]ethoxy]ethoxy]ethanol (6.30 g, 13.6 mmol, 1.0 equiv) at anhydrous condition and 0° C., after stirred at 25° C. for 30 mins. Then 2-bromo-1,1-dimethoxy-ethane (4.62 g, 27.3 mmol, 3.21 mL, 2.0 equiv) was added at 0° C., and it was stirred at 25° C. for 12 h. The reaction mixture was dropwise added to saturated NH₄Cl (100 mL), extracted with ethyl acetate (2×100 mL), dried, filtered and concentrated to afford a crude product. The crude product was purified by column chromatography (SiO₂, PE:EA=1:1 to 0:1) to afford the desired product 2-[2-[2-[2-[2-[2-[2-[2-(2,2-dimethoxyethoxy)ethoxy]ethoxy]ethoxy]ethoxy]ethoxy]ethoxy]ethoxy]ethoxymethylbenzene (3.90 g, 7.11 mmol, 51% yield) as a colorless oil. 1H NMR (400 MHz, CDCl₃) δ (ppm) 7.38-7.24 (m, 5H), 4.57 (s, 2H), 4.52 (t, J=5.3 Hz, 1H), 3.70-3.62 (m, 32H), 3.55 (d, J=5.2 Hz, 2H), 3.40 (s, 6H). LC-MS: MS (ES⁺): RT=0.867 min, m/z=566.3 [M+H₃O⁺].

### 3. Preparation of Compound 4

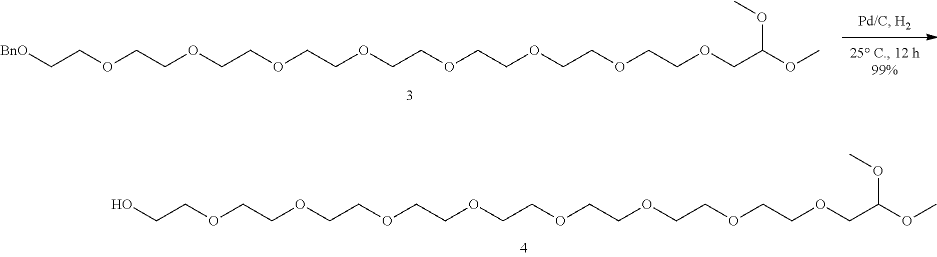 [SMILES: COC(COCCOCCOCCOCCOCCOCCOCCOCCOCc1ccccc1)OC ; COC(COCCOCCOCCOCCOCCOCCOCCOCCO)OC]

To a solution of 2-[2-[2-[2-[2-[2-[2-[2-(2,2-dimethoxyethoxy)ethoxy]ethoxy]ethoxy]ethoxy]ethoxy]ethoxy]ethoxy]ethoxymethylbenzene (3.90 g, 7.11 mmol, 1.0 equiv) in THF (40 mL) was added Pd/C (500 mg, 10% purity, 1.0 equiv) under N₂. The suspension was degassed under vacuum and purged with H₂ several times. The mixture was stirred under H₂ (50 psi) at 25° C. for 12 h. The reaction mixture was filtered and the filtrate was concentrated to give the desired product 2-[2-[2-[2-[2-[2-[2-[2-(2,2-dimethoxyethoxy)ethoxy]ethoxy]ethoxy]ethoxy]ethoxy]ethoxy]ethoxy]ethanol (3.26 g, 7.11 mmol, 99% yield) as a colorless oil. ¹H NMR (400 MHz, CDCl₃) δ (ppm) 4.52 (t, J=5.3 Hz, 1H), 3.75-3.71 (m, 2H), 3.69-3.59 (m, 30H), 3.55 (d, J=5.2 Hz, 2H), 3.40 (s, 6H).

### 4. Preparation of Compound 5

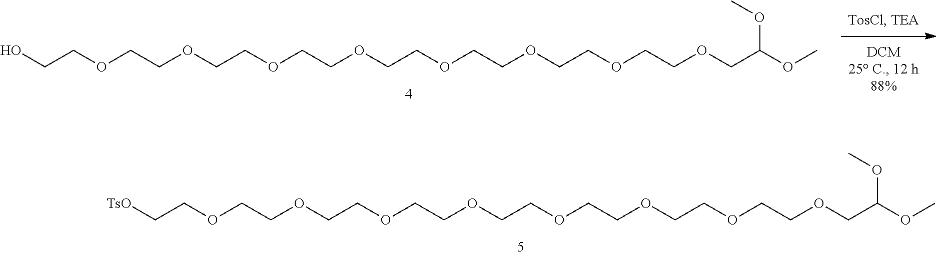 [SMILES: COC(COCCOCCOCCOCCOCCOCCOCCOCCO)OC ; COC(COCCOCCOCCOCCOCCOCCOCCOCCOS(=O)(=O)c1ccc(C)cc1)OC]

To a solution of 2-[2-[2-[2-[2-[2-[2-[2-(2,2-dimethoxyethoxy)ethoxy]ethoxy]ethoxy]ethoxy]ethoxy]ethoxy]ethoxy]ethanol (3.26 g, 7.11 mmol, 1.0 equiv) in DCM (30 mL) was added TEA (1.80 g, 17.7 mmol, 2.47 mL, 2.5 equiv), 4-methylbenzenesulfonyl chloride (2.71 g, 14.2 mmol, 2.0 equiv) at 0° C. The solution was stirred at 25° C. for 12 h. The reaction mixture was washed with water (2×50 mL), and the combined water phase was extracted with ethyl acetate (2×100 mL). The combined organic layers were dried, filtered and concentrated to afford a crude product. The crude product was purified by column chromatography (SiO₂, PE:EA=5:1 to 0:1) to give the desired product 2-[2-[2-[2-[2-[2-[2-[2-(2,2-dimethoxyethoxy)ethoxy]ethoxy]ethoxy]ethoxy]ethoxy]ethoxy]ethoxy]ethyl 4-methylbenzene-sulfonate (3.4 g, 4.8 mmol, 68% yield, 88% purity) as a colorless oil. ¹H NMR (400 MHz, CDCl₃) δ (ppm) 7.92-7.74 (d, J=8.0 Hz, 2H), 7.35 (d, J=8.0 Hz, 2H), 4.52 (t, J=5.2 Hz, 1H), 4.20-4.12 (m, 2H), 3.72-3.57 (m, 32H), 3.40 (s, 6H), 2.46 (s, 3H). LC-MS: MS (ES⁺): m/z=630.2 [M+H₂O].

### 5. Preparation of Compound 6

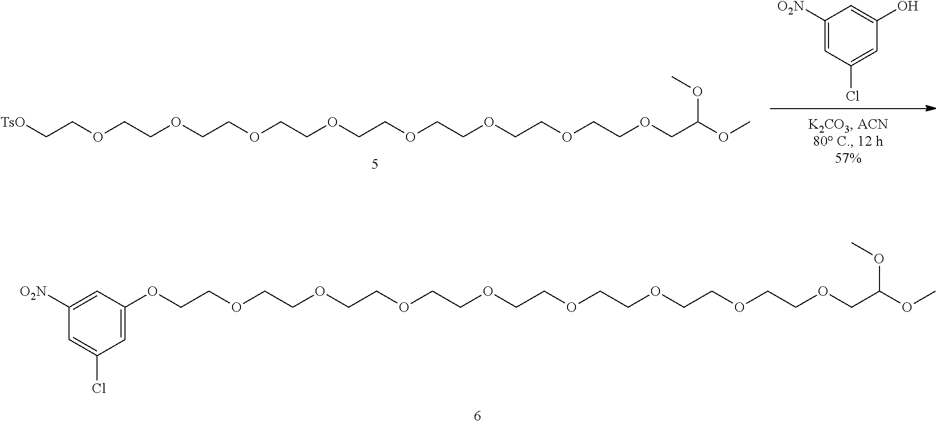 [SMILES: COC(COCCOCCOCCOCCOCCOCCOCCOCCOS(=O)(=O)c1ccc(C)cc1)OC ; O=[N+]([O-])c1cc(O)cc(Cl)c1 ; COC(COCCOCCOCCOCCOCCOCCOCCOCCOc1cc(Cl)cc([N+](=O)[O-])c1)OC]

To a solution of 2-[2-[2-[2-[2-[2-[2-[2-(2,2-dimethoxyethoxy)ethoxy]ethoxy]ethoxy]ethoxy]ethoxy]ethoxy]ethoxy]ethyl 4-methylbenzenesulfonate (500 mg, 816 μmol, 1.0 equiv) in CH₃CN (10 mL) was added 3-chloro-5-nitro-phenol (141.62 mg, 816.03 μmol, 1.0 equiv), K₂CO₃ (338 mg, 2.45 mmol, 3.0 equiv), then it was stirred at 80° C. for 12 h. The reaction mixture was filtered and the filtrate was concentrated to give a residue. The residue was purified by column chromatography (SiO₂, PE:EA=0:1) to give the desired product 1-chloro-3-[2-[2-[2-[2-[2-[2-[2-[2-(2,2-dimethoxyethoxy)ethoxy]ethoxy]ethoxy]ethoxy]ethoxy]ethoxy]ethoxy]ethoxy]-5-nitro-benzene (290 mg, 472 μmol, 57% yield) as a colorless oil. ¹H NMR (400 MHz, CDCl₃) δ (ppm) 7.83 (t, J=1.9 Hz, 1H), 7.68 (t, J=2.2 Hz, 1H), 7.27-7.25 (m, 1H), 4.53 (t, J=5.3 Hz, 1H), 4.24-4.19 (m, 2H), 3.91-3.86 (m, 2H), 3.71-3.62 (m, 28H), 3.55 (d, J=5.2 Hz, 2H), 3.40 (s, 6H). LC-MS: MS (ES⁺): RT=0.883 min, m/z=631.2 [M+18].

### 6. Preparation of Compound 7

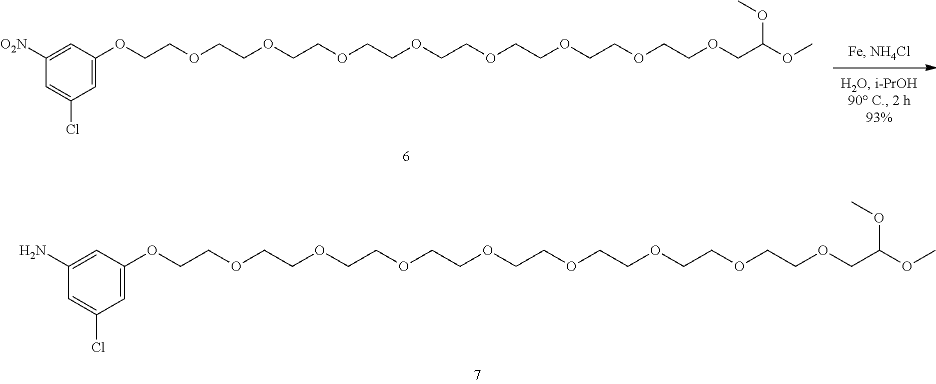 [SMILES: COC(COCCOCCOCCOCCOCCOCCOCCOCCOc1cc(Cl)cc([N+](=O)[O-])c1)OC ; COC(COCCOCCOCCOCCOCCOCCOCCOCCOc1cc(N)cc(Cl)c1)OC]

To a solution of 1-chloro-3-[2-[2-[2-[2-[2-[2-[2-[2-(2,2-dimethoxyethoxy) ethoxy]ethoxy]ethoxy]ethoxy]ethoxy]ethoxy]ethoxy]ethoxy]-5-nitro-benzene (280 mg, 455 μmol, 1.0 equiv) in i-PrOH (2 mL) was added NH₄Cl (243 mg, 4.56 mmol, 10.0 equiv) in H₂O (0.5 mL), then Fe (127 mg, 2.28 mmol, 5.0 equiv) was added, and it was stirred at 90° C. for 2 h. The reaction mixture was diluted with EtOAc (20 mL) and water (10 mL). The mixture was filtered. The filtrate was extracted with EtOAc (2×20 mL), and the combined organic layers were dried, filtered and concentrated to give a residue. The residue was purified by column chromatography (SiO₂, PE:EA=1:1 to 0:1) to give the desired product 3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethoxy]aniline (250 mg, 428 μmol, 93% yield) as colorless oil. LC-MS: MS (ES⁺): m/z=584.3 [M+H⁺].

### 7. Preparation of Compound 9

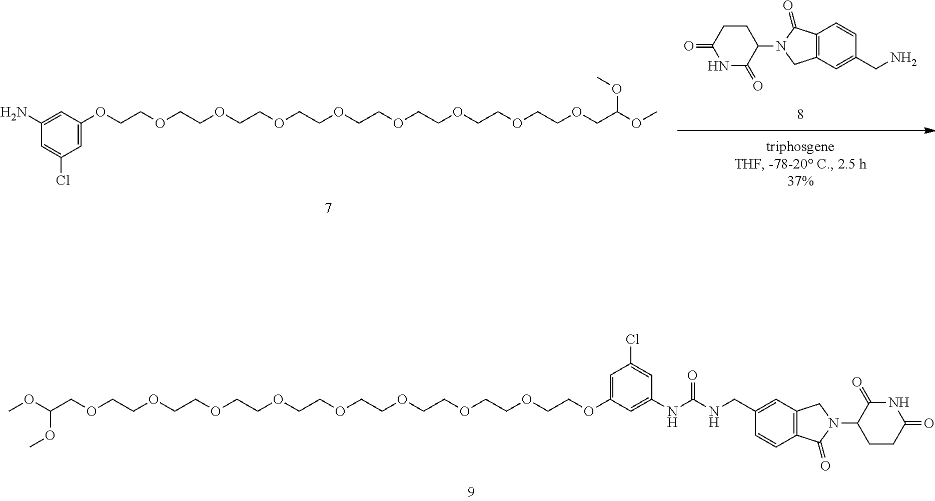

To a solution of triphosgene (90.0 mg, 303 μmol, 1.0 equiv) in DCM (3 mL) was add TEA (346 mg, 3.42 mmol, 476 μL, 10.0 equiv), followed by 3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethoxy]aniline (200 mg, 342 μmol, 1.0 equiv) in DCM (3 mL) at −78° C. The mixture was stirred at −78° C. for 0.5 h. Then 3-[5-(aminomethyl)-1-oxo-isoindolin-2-yl]piperidine-2,6-dione (127.27 mg, 410.89 μmol, 1.2 equiv, HCl salt) was added. The solution was stirred at 20° C. for 2 h. The reaction mixture was poured into saturated NaHCO₃ (20 mL), extracted with DCM (2×20 mL), dried over Na₂SO₄, filtered and concentrated to afford a crude product. The crude product was purified by prep-TLC (SiO₂ DCM:MeOH=10:1) to give the desired product 1-[3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (112 mg, 126 μmol, 37% yield) as a colorless oil. LC-MS: MS (ES⁺): m/z=900.2 [M+18].

### 8. Preparation of Compound 10

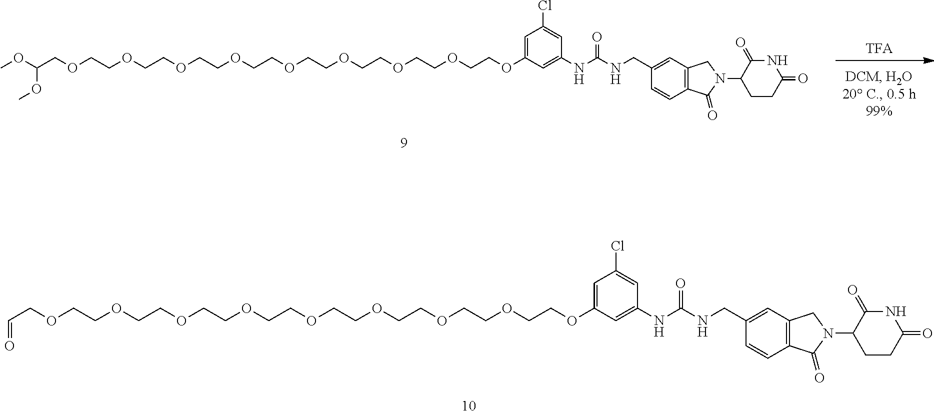 [SMILES: COC(COCCOCCOCCOCCOCCOCCOCCOCCOc1cc(Cl)cc(NC(=O)NCc2ccc3c(c2)CN(C2CCC(=O)NC2=O)C3=O)c1)OC ; O=CCOCCOCCOCCOCCOCCOCCOCCOCCOc1cc(Cl)cc(NC(=O)NCc2ccc3c(c2)CN(C2CCC(=O)NC2=O)C3=O)c1 ; "TFA"]

A mixture of 1-[3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (60 mg, 67 μmol, 1.0 equiv) in DCM (2 mL), TFA (0.2 mL) and H₂O (0.04 mL) was stirred at 20° C. for 0.5 h. The reaction mixture was adjusted with Et₃N to pH=7˜8 at 0° C. The mixture of desired product 1-[3-chloro-5-[2-[2-[2-[2-[2-[2-[2-[2-(2-oxoethoxy)ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (64.62 mg, 67.93 μmol, 100% yield, TFA salt) as a yellow solution was used into the next step without further purification. LC-MS: MS (ES⁺): m/z=837.6 [M+18].

### 9. Preparation of Compound I-9

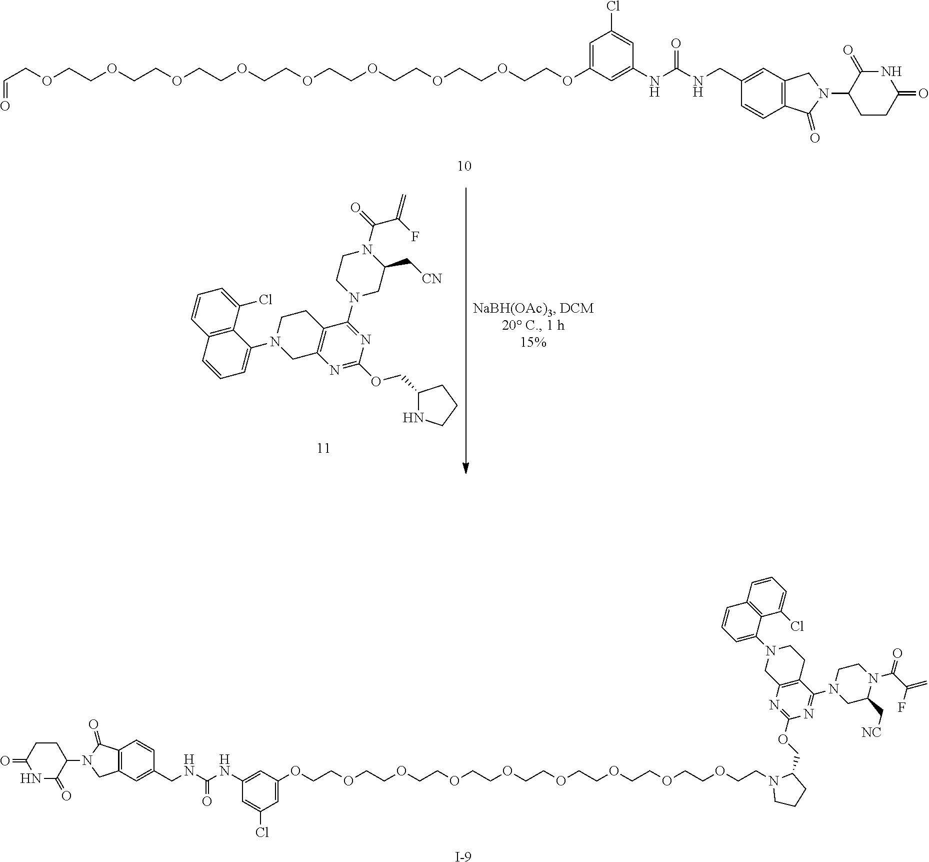

To a solution of 1-[3-chloro-5-[2-[2-[2-[2-[2-[2-[2-[2-(2-oxoethoxy)ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (56 mg, 66 μmol, 1.0 equiv) in DCM (3 mL) was added NaBH(OAc)₃ (141.75 mg, 668.81 μmol, 10.0 equiv), 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (47.09 mg, 66.88 μmol, 1.0 equiv, TFA salt). The solution was stirred at 20° C. for 1 h. The reaction mixture was partitioned between H₂O (10 mL) and DCM (20 mL). The organic phase was separated, dried over Na₂SO₄, filtered and concentrated under reduced pressure to give a residue. The residue was purified by prep-HPLC (column: Phenomenex Gemini-NX C18 75*30 mm*3 μm; mobile phase: [water (10 mM NH₄HCO₃)-ACN]; B %: 40%-70%, 8 min) to give the desired product 1-[3-chloro-5-[2-[2-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (14.44 mg, 10.23 μmol, 15% yield) as a white solid. ¹H NMR (400 MHz, CD₃OD) δ (ppm) 7.85-7.74 (m, 2H), 7.69 (d, J=8.6 Hz, 1H), 7.58-7.43 (m, 4H), 7.42-7.28 (m, 2H), 7.09 (s, 1H), 7.00 (s, 1H), 6.59 (d, J=1.6 Hz, 1H), 5.45-5.22 (m, 2H), 5.15 (dd, J=5.2, 13.2 Hz, 1H), 4.60 (br d, J=3.2 Hz, 1H), 4.51 (s, 2H), 4.48-4.32 (m, 3H), 4.32-4.13 (m, 4H), 4.13-4.05 (m, 2H), 3.88-3.78 (m, 2H), 3.77-3.49 (m, 33H), 3.49-3.39 (m, 1H), 3.27-3.00 (m, 7H), 2.99-2.85 (m, 3H), 2.83-2.56 (m, 3H), 2.51-2.37 (m, 2H), 2.21-2.12 (m, 1H), 2.07-2.01 (m, 1H), 1.91-1.57 (m, 3H). LC-MS: MS (ES⁺): RT=2.317 min, m/z=1412.6 [M+H⁺].

### Example 10—Preparation of Compound I-10

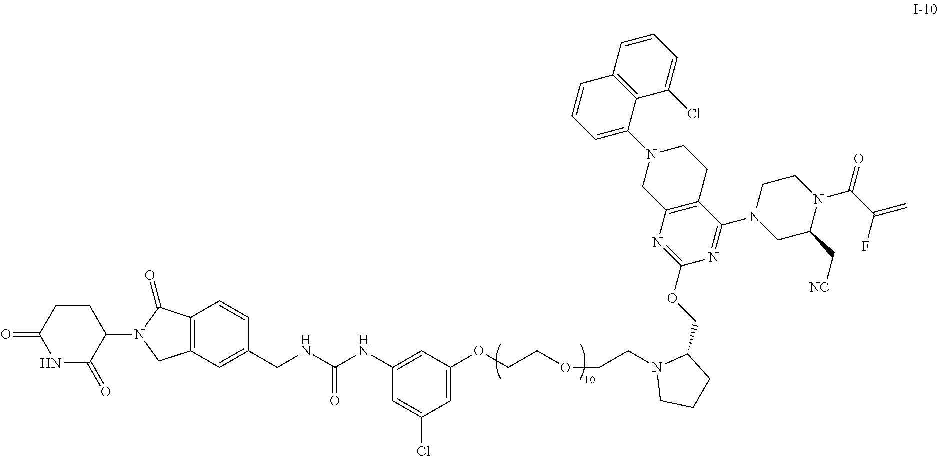

The title compound was prepared according to the following procedures.

### 1. Preparation of Compound 2

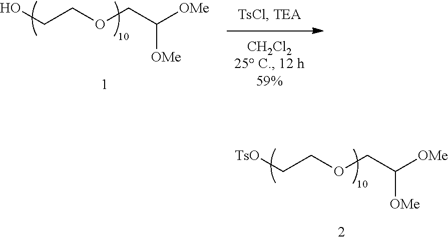

To a solution of 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethanol (1.20 g, 2.20 mmol, 1.0 equiv) in CH₂Cl₂ (20 mL) were added TosCl (0.63 g, 3.29 mmol, 1.5 equiv) and Et₃N (0.44 g, 4.39 mmol, 0.61 mL, 2.0 equiv) at 25° C. under N₂. The mixture was stirred at 25° C. for 12 h. The reaction mixture was concentrated in vacuo. The residue product was purified by column chromatography on silica gel (Dichloromethane:Methanol=1:0 to 10:1) to afford 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl 4-methylbenzenesulfonate (0.90 g, 1.28 mmol, 59% yield) as a yellow oil. ¹H NMR (400 MHz, CDCl₃): δ 7.90-7.75 (m, 2H), 7.39-7.30 (m, 2H), 4.51 (t, 1H, J=5.2 Hz), 4.31-4.13 (m, 2H), 3.76-3.65 (m, 38H), 3.56-3.52 (m, 2H), 3.39 (s, 6H), 2.45 (s, 3H).

### 2. Preparation of Compound 3

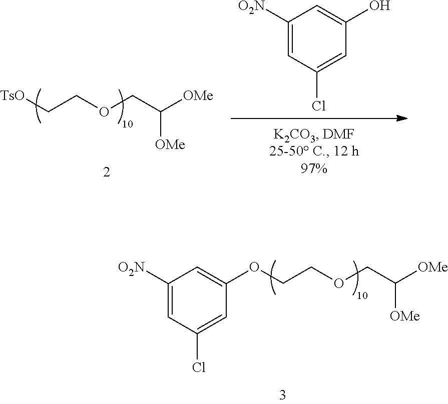

To a solution of 3-chloro-5-nitro-phenol (175 mg, 1.01 mmol, 1.2 equiv) and 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl 4-methylbenzenesulfonate (590 mg, 0.84 mmol, 1.0 equiv) in DMF (5 mL) was added K₂CO₃ (233 mg, 1.68 mmol, 2.0 equiv) at 25° C. under N₂. The mixture was stirred at 50° C. for 12 h. The reaction mixture was diluted with water (10 mL) and the mixture was extracted with ethyl acetate (20 mL*3). The combined organic phase was washed with brine (20 mL), dried over anhydrous Na₂SO₄, filtered and concentrated in vacuo to afford 1-chloro-3-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethoxy]-5-nitro-benzene (560 mg, 0.80 mmol, 95% yield) as a yellow oil. ¹H NMR (400 MHz, CDCl₃): δ 7.85-7.78 (m, 1H), 7.69-7.65 (m, 1H), 7.26-7.24 (m, 1H), 4.51 (t, 1H, J=5.2 Hz), 4.21-4.19 (m, 2H), 3.90-3.84 (m, 2H), 3.68-3.60 (m, 38H), 3.39 (s, 6H).

### 3. Preparation of Compound 4

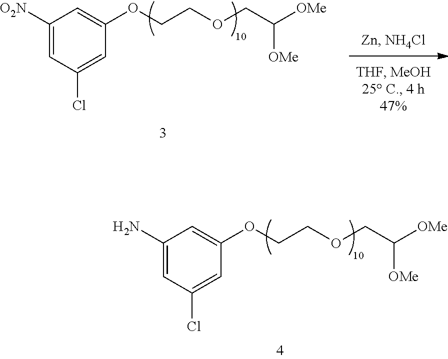

A mixture of 1-chloro-3-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethoxy]-5-nitro-benzene (760 mg, 1.08 mmol, 1.0 equiv), Zn (354 mg, 5.41 mmol, 5.0 equiv) and NH₄Cl (289 mg, 5.41 mmol, 5.0 equiv) in THF (5 mL) and MeOH (5 mL) was stirred at 25° C. for 3 h. The mixture was filtered and the filtrate was concentrated in vacuo. To the residue were added THF (5 mL), MeOH (5 mL), Zn (354 mg, 5.41 mmol, 5.0 equiv) and NH₄Cl (289 mg, 5.41 mmol, 5.0 equiv). The mixture was stirred at 25° C. for 1 h. The reaction mixture was filtered. The filtrate was diluted with water (20 mL) and the mixture was extracted with CH₂Cl₂ (30 mL*3). The combined organic phase was washed with brine (20 mL), dried over anhydrous Na₂SO₄, filtered and concentrated in vacuo. The residue was purified by column chromatography on silica gel (Dichloromethane:Methanol=1:0 to 10:1) and then prep-HPLC (column: Waters Xbridge C18 150*50 mm*10 μm; mobile phase: [water (10 mM NH₄HCO₃)-ACN]; B %: 22%-52%) to afford 3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethoxy]aniline (340 mg, 0.51 mmol, 47% yield) as a yellow oil. LC-MS: MS (ES⁺): m/z=672.5 [M+H⁺].

### 4. Preparation of Compound 5

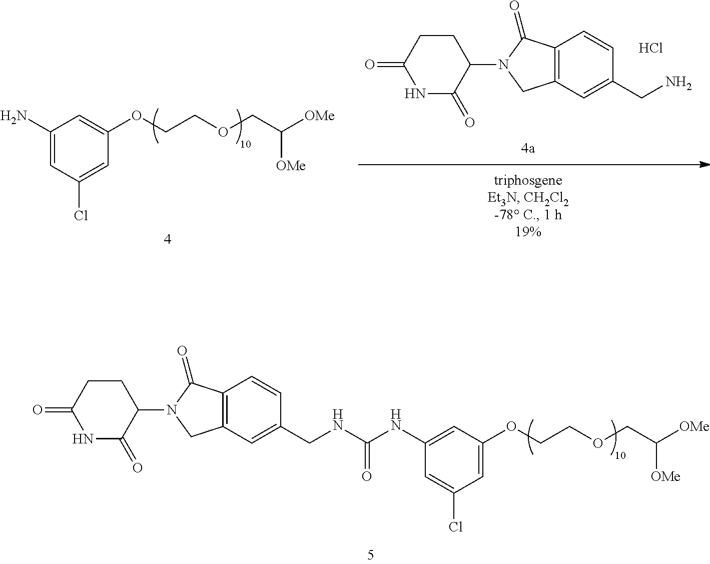

To a solution of bis(trichloromethyl) carbonate (124 mg, 0.417 mmol, 1.0 equiv) in CH₂Cl₂ (30 mL) were added Et₃N (422 mg, 4.17 mmol, 0.58 mL, 10.0 equiv) and 3-[5-(aminomethyl)-1-oxo-isoindolin-2-yl]piperidine-2,6-dione (155 mg, 0.50 mmol, 1.2 equiv, HCl salt) in CH₂Cl₂ (5 mL) at −78° C. The mixture was stirred at −78° C. for 0.5 h. To the mixture was added 3-chloro-5-[2-[2-[2-[2-[2-[2-[2-[2-[2-[2-(2,2-dimethoxyethoxy) ethoxy]ethoxy]ethoxy]ethoxy]ethoxy]ethoxy]ethoxy]ethoxy]ethoxy]ethoxy]aniline (280 mg, 0.417 mmol, 1.0 equiv) at −78° C. and the mixture was tirred at 20° C. for 0.5 h. The reaction mixture was poured into sat. aq. NaHCO₃ (30 mL) at 0° C. and the mixture was extracted with DCM (30 mL). The combined organic phase was washed with brine (30 mL), dried over Na₂SO₄, filtered and concentrated in vacuo. The residue was purified by prep-TLC on silica gel (Dichloromethane:Methanol=15:1) to afford 1-[3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (76.0 mg, 0.78 mmol, 19% yield) as a yellow oil. ¹H NMR (400 MHz, CDCl₃): δ 8.17-8.09 (m, 1H), 7.82-7.74 (m, 1H), 7.50 (s, 1H), 7.43-7.39 (m, 1H), 7.34 (s, 1H), 6.95-6.89 (m, 1H), 6.50 (s, 1H), 5.22-5.11 (m, 1H), 4.52-4.46 (m, 3H), 4.13-4.05 (m, 2H), 3.82-3.77 (m, 2H), 3.69-3.66 (m, 2H), 3.63-3.54 (m, 38H), 3.37 (s, 6H), 3.00-2.66 (m, 2H), 2.47-2.07 (m, 2H). LC-MS: MS (ES⁺): m/z=988.6 [M+H⁺+17].

### 5. Preparation of Compound 6

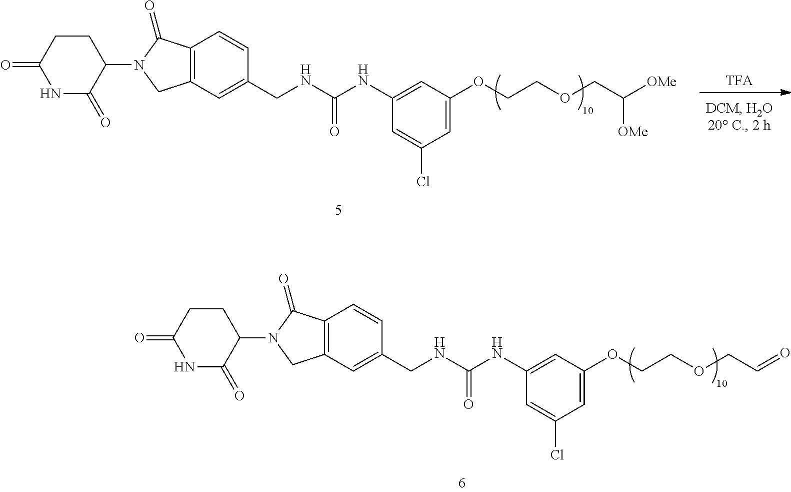

A mixture of 1-[3-chloro-5-[2-[2-[2-[2-[2-[2-[2-[2-[2-[2-(2,2-dimethoxyethoxy) ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (66.0 mg, 0.68 mmol, 1.0 equiv) in DCM (3 mL), H₂O (0.06 mL) and TFA (0.3 mL) was stirred at 20° C. for 2 h. The pH of the mixture was adjusted to 7˜8 at 0° C. by addition of Et₃N. The solution was used directly in the next step. LC-MS: MS (ES⁺): m/z=925.3 [M+H⁺].

### 6. Preparation of I-10

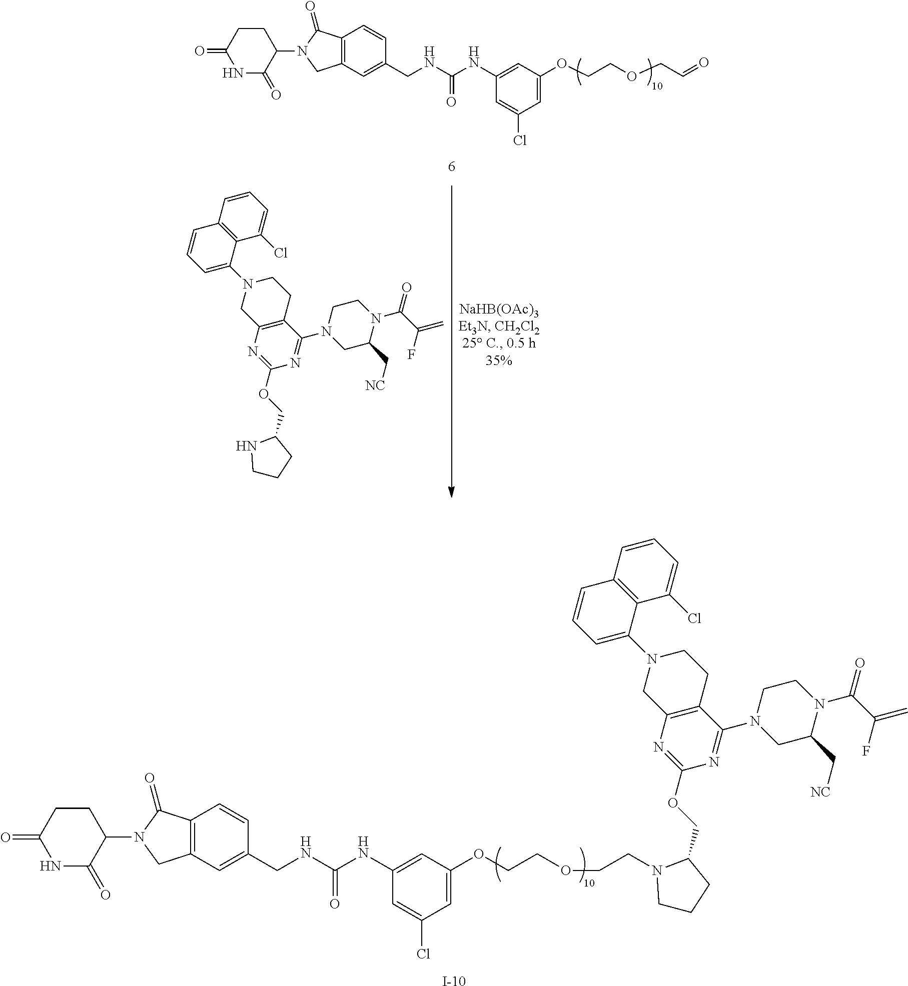

To a solution of 1-[3-chloro-5-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (62.0 mg, 0.07 mmol, 1.05 equiv) in CH₂Cl₂ (3 mL) were added Et₃N (19.4 mg, 0.192 mmol, 0.03 mL, 3.0 equiv), 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (45.0 mg, 0.064 mmol, 1.0 equiv, TFA salt) and NaBH(OAc)₃ (136 mg, 0.639 mmol, 10.0 equiv) at 20° C. The reaction mixture was stirred at 20° C. for 0.5 h. The reaction mixture was diluted with water (10 mL) and the mixture was extracted with dichloromethane (15 mL*3). The combined organic phase was washed with brine (10 mL), dried with anhydrous Na₂SO₄, filtered and concentrated in vacuo. The residue product was purified by prep-HPLC (column: Phenomenex Synergi C18 150*25 mm*10 μm; mobile phase: [water (0.225% FA)-ACN]; B %: 27%-57%, 10 min) to afford 1-[3-chloro-5-[2-[2-[2-[2-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (34.4 mg, 0.023 mmol, 35% yield, 98% purity) as a white solid. ¹H NMR (400 MHz, DMSO-d₆): δ 10.99 (s, 1H), 9.03 (s, 1H), 7.91 (d, 1H, J=8.0 Hz), 7.74-7.72 (m, 1H), 7.72-7.64 (m, 1H), 7.63-7.56 (m, 1H), 7.56-7.48 (m, 2H), 7.47-7.39 (m, 2H), 7.38-7.28 (m, 1H), 7.22-7.14 (m, 1H), 7.11-7.01 (m, 1H), 6.99-6.92 (m, 1H), 6.59-6.51 (m, 1H), 5.43-5.36 (m, 1H), 5.35-5.18 (m, 1H), 5.15-5.06 (m, 1H), 4.48-4.35 (m, 3H), 4.34-4.26 (m, 1H), 4.24-4.14 (m, 2H), 4.09-4.00 (m, 3H), 3.98-3.86 (m, 2H), 3.84-3.59 (m, 4H), 3.58-3.43 (m, 40H), 3.28-2.85 (m, 10H), 2.82-2.73 (m, 1H), 2.71-2.55 (m, 2H), 2.45-2.31 (m, 2H), 2.26-2.20 (m, 1H), 2.03-1.95 (m, 1H), 1.91-1.81 (m, 1H), 1.72-1.62 (m, 2H), 1.60-1.51 (m, 1H). LC-MS: MS (ES⁺): RT=2.923 min, m/z=749.8 [1/2M+H⁺]; LCMS method: LC-MS METHOD 01.

### Example 11—Preparation of Compound I-11

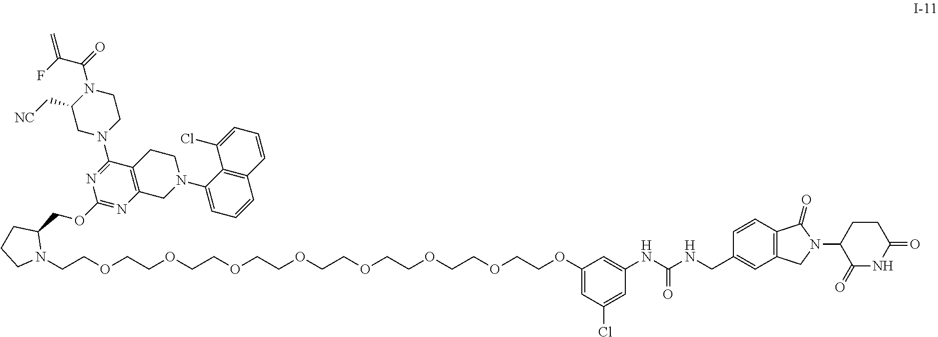 [SMILES: C=C(F)C(=O)N1CCN(c2nc(OC[C@@H]3CCCN3CCOCCOCCOCCOCCOCCOCCOCCOc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N]

The title compound was prepared according to the following procedures.

### 1. Preparation of Compound 2

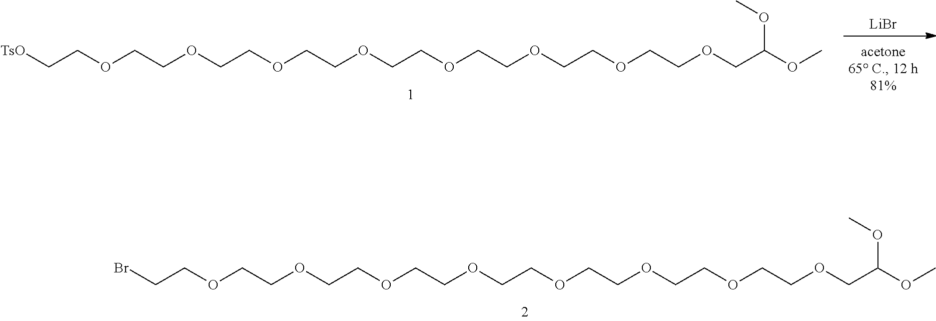 [SMILES: COC(COCCOCCOCCOCCOCCOCCOCCOCCOS(=O)(=O)c1ccc(C)cc1)OC ; "LiBr" ; COC(COCCOCCOCCOCCOCCOCCOCCOCCBr)OC]

To a solution of 2-[2-[2-[2-[2-[2-[2-[2-(2,2-dimethoxyethoxy)ethoxy]ethoxy]ethoxy]ethoxy]ethoxy]ethoxy]ethoxy]ethyl 4-methylbenzenesulfonate (2.6 g, 4.2 mmol, 1.0 equiv) in acetone (30 mL) was added LiBr (1.84 g, 21.2 mmol, 532 μL, 5.0 equiv). The solution was stirred at 65° C. for 12 h. The reaction mixture was filtered and the filtrate was concentrated to give a crude product. The crude product was purified by column chromatography (SiO₂, PE:EA=1:1 to 0:1) to give the product 2-[2-[2-[2-[2-[2-[2-[2-(2-bromoethoxy)ethoxy]ethoxy]ethoxy]ethoxy]ethoxy]ethoxy]ethoxy]-1,1-dimethoxy-ethane (1.8 g, 3.4 mmol, 81% yield) as a colorless oil. ¹H NMR (400 MHz, CDCl₃) δ 4.54 (m, 1H), 3.82 (t, J=6.4 Hz, 2H), 3.65-3.70 (m, 28H), 3.55 (d, J=5.3 Hz, 2H), 3.48 (t, J=6.3 Hz, 2H), 3.40 (s, 6H). LC-MS: MS (ES⁺): m/z=540.2 [M+H₃O⁺].

### 2. Preparation of Compound 4

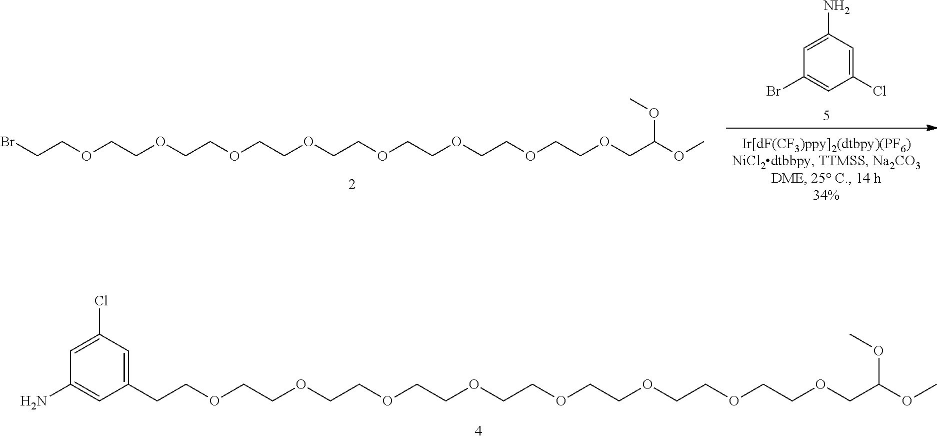

To an 15 mL vial equipped with a stir bar was added 3-bromo-5-chloro-aniline (712 mg, 3.45 mmol, 1.0 equiv), 2-[2-[2-[2-[2-[2-[2-[2-(2-bromoethoxy)ethoxy]ethoxy]ethoxy]ethoxy]ethoxy]ethoxy]ethoxy]-1,1-dimethoxy-ethane (1.8 g, 3.4 mmol, 1.0 equiv), Ir[dF(CF₃)ppy]₂(dtbpy)(PF₆) (38 mg, 34 μmol, 0.01 equiv), NiCl₂.dtbbpy (68.69 mg, 172.6 μmol, 0.05 equiv), TTMSS (858 mg, 3.45 mmol, 1.06 mL, 1.0 equiv), Na₂CO₃ (731 mg, 6.90 mmol, 2.0 equiv) in DME (40 mL). The vial was sealed and placed under nitrogen. The reaction was stirred and irradiated with a 34 W blue LED lamp (7 cm away), with cooling fan to keep the reaction temperature at 25° C. for 14 h. The reaction mixture was filtered and concentrated under reduced pressure to give a residue. The residue was purified by column chromatography (SiO₂, Petroleum ether/Ethyl acetate=1/1 to EA/MeOH=10/1) and prep-HPLC (column: Waters Xbridge BEH C18 250*50 mm*10 μm; mobile phase: [water (10 mM NH₄HCO₃)-ACN]; B %: 30%-60%, 13 min) to give the product 3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethyl]aniline (678 mg, 1.19 mmol, 34% yield) as a colorless oil. LC-MS: MS (ES⁺): m/z=568.3 [M+H⁺].

### 3. Preparation of Compound 6

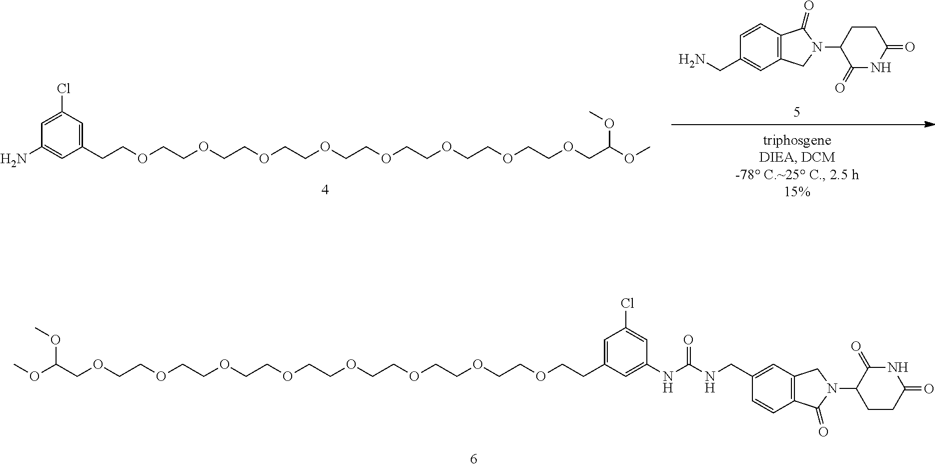

To a solution of triphosgene (156.71 mg, 528.08 μmol, 1.0 equiv) in DCM (5 mL) at −78° C. was added TEA (534.3 mg, 5.280 mmol, 735.0 μL, 10.0 equiv) and 3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethyl]aniline (300 mg, 528 μmol, 1.0 equiv) in DCM (5 mL) at −78° C. The mixture was stirred at −78° C. for 0.5 h. Then 3-[5-(aminomethyl)-1-oxo-isoindolin-2-yl]piperidine-2,6-dione (196.29 mg, 633.70 μmol, 1.2 equiv, HCl salt) was added at −78° C. The mixture was stirred at 25° C. for 2 h. The reaction mixture was poured into saturated NaHCO₃ (20 mL), extracted with DCM (2×20 mL), dried over Na₂SO₄, filtered and concentrated to afford a crude product. The crude product was purified by prep-TLC (SiO₂ DCM:MeOH=10:1) to give the desired product 1-[3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (70 mg, 46 μmol, 15% yield) as a yellow gum. LC-MS: MS (ES⁺): m/z=884.5 [M+18].

### 4. Preparation of Compound 7

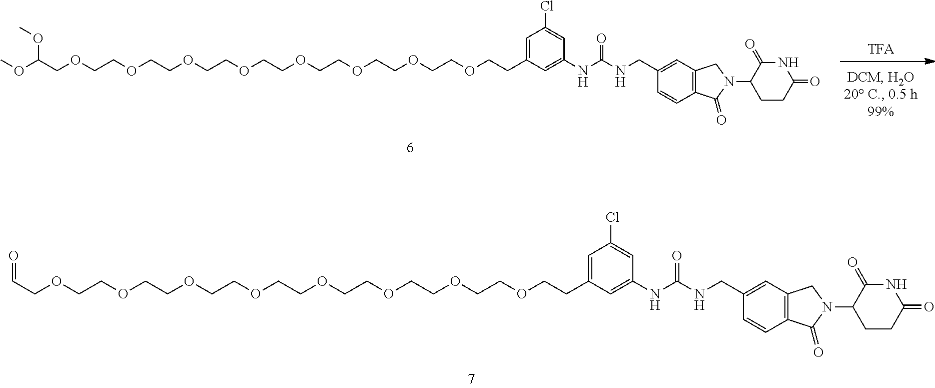 [SMILES: COC(COCCOCCOCCOCCOCCOCCOCCOCCc1cc(Cl)cc(NC(=O)NCc2ccc3c(c2)CN(C2CCC(=O)NC2=O)C3=O)c1)OC ; "TFA" ; O=CCOCCOCCOCCOCCOCCOCCOCCOCCc1cc(Cl)cc(NC(=O)NCc2ccc3c(c2)CN(C2CCC(=O)NC2=O)C3=O)c1]

A mixture of 1-[3-chloro-5-[2-[2-[2-[2-[2-[2-[2-[2-(2,2-dimethoxyethoxy)ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (70 mg, 80 μmol, 1.0 equiv) in DCM (2 mL), TFA (0.2 mL) and H₂O (0.04 mL) was stirred at 25° C. for 0.5 h. The reaction mixture was adjusted with Et₃N to pH=7-8 at 0° C. The mixture of desired product 1-[3-chloro-5-[2-[2-[2-[2-[2-[2-[2-[2-(2-oxoethoxy)ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (66.28 mg, crude) as a yellow solution was used into the next step without further purification. LC-MS: MS (ES⁺): m/z=821.4 [M+H⁺].

### 5. Preparation of I-11

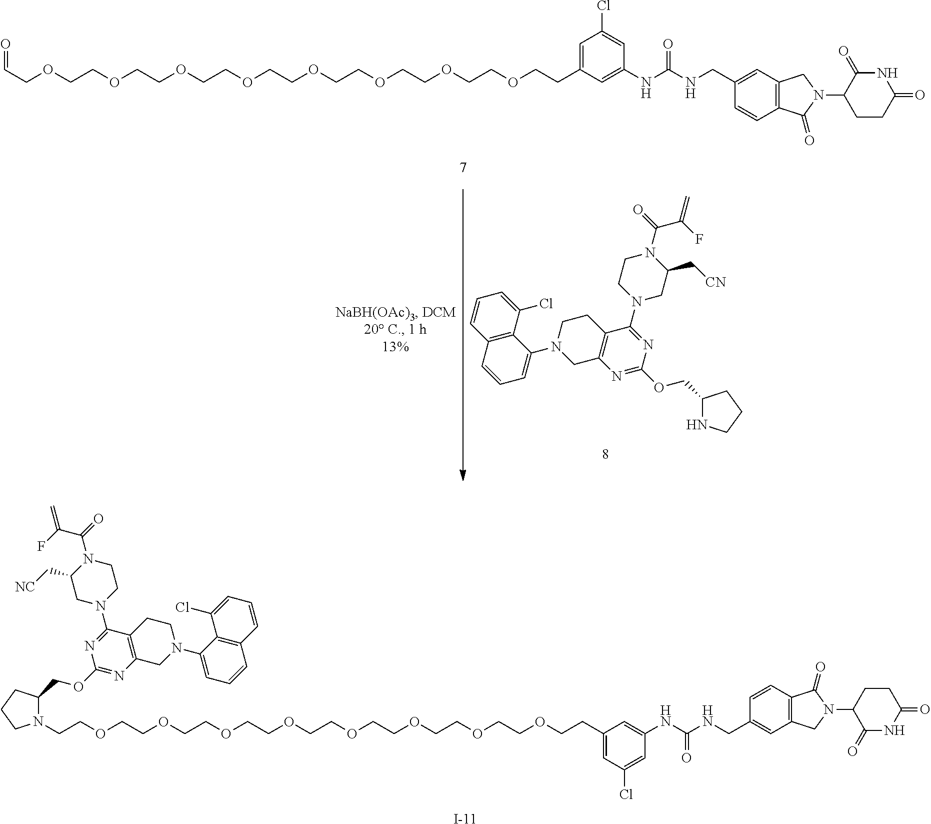

To a solution of 1-[3-chloro-5-[2-[2-[2-[2-[2-[2-[2-[2-(2-oxoethoxy)ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (66 mg, 80 μmol, 1.0 equiv), 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (67.90 mg, 96.43 μmol, 1.2 equiv, TFA salt) in DCM (4 mL) was added NaBH(OAc)₃ (170.31 mg, 803.60 μmol, 10.0 equiv). The mixture was stirred at 20° C. for 1 h. The reaction mixture was partitioned between H₂O (10 mL) and DCM (20 mL). The organic phase was separated, dried over Na₂SO₄, filtered and concentrated under reduced pressure to give a residue. The residue was purified by prep-HPLC (column: Phenomenex Gemini-NX C18 75*30 mm*3 μm; mobile phase: [water (10 mM NH₄HCO₃)-ACN]; B %: 42%-72%, 8 min) to give the product 1-[3-chloro-5-[2-[2-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (15 mg, 10 μmol, 14% yield) as an off-white solid. ¹H NMR (400 MHz, CD₃OD) δ ppm 7.81 (d, J=8.2 Hz, 1H), 7.75 (d, J=8.0 Hz, 1H), 7.67 (d, J=8.1 Hz, 1H), 7.56-7.43 (m, 5H), 7.39-7.28 (m, 2H), 7.11 (s, 1H), 6.86 (s, 1H), 5.40-5.24 (m, 2H), 5.13 (m, 1H), 4.50 (s, 2H), 4.46-4.00 (m, 7H), 3.56 (m, 36H), 3.23-2.56 (m, 16H), 2.51-2.34 (m, 2H), 2.23-2.08 (m, 1H), 2.06-1.95 (m, 1H), 1.86-1.65 (m, 3H). LC-MS: MS (ES⁺): RT=2.370 min, m/z=698.0 [M/2+H⁺].

### Example 12—Preparation of Compound I-12

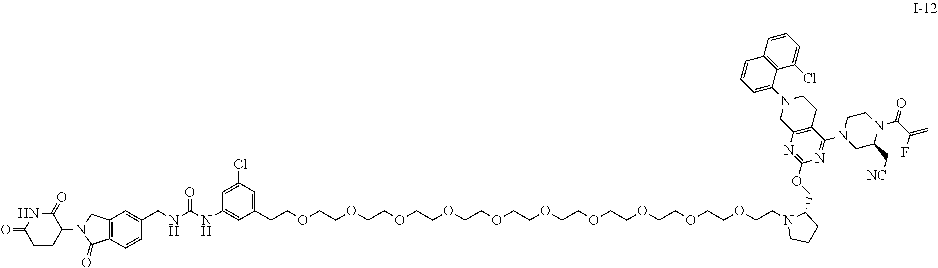 [SMILES: C=C(F)C(=O)N1CCN(c2nc(OC[C@@H]3CCCN3CCOCCOCCOCCOCCOCCOCCOCCOCCOCCOCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N]

The title compound was prepared according to the following procedures.

### 1. Preparation of Compound 2

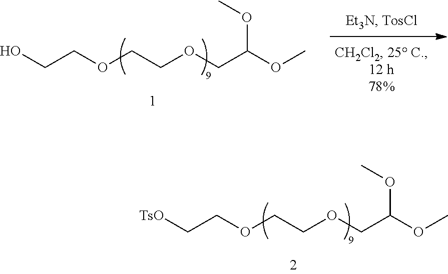

To a solution of 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethanol (1.2 g, 2.2 mmol, 1.0 equiv) in CH₂Cl₂ (10 mL) was added TEA (444 mg, 4.39 mmol, 611 μL, 2.0 equiv) and TosCl (628 mg, 3.29 mmol, 1.5 equiv), and then it was stirred at 25° C. for 12 h. The reaction mixture was concentrated to afford crude product. The residue was purified by silica chromatography (EtOAc:MeOH=1:0-20:1) to afford 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl 4-methylbenzenesulfonate (1.2 g, 1.7 mmol, 78% yield) as a colorless oil. ¹H NMR (400 MHz, CDCl₃) δ 7.80 (d, J=8.2 Hz, 2H), 7.35 (d, J=8.2 Hz, 2H), 4.52 (t, J=5.2 Hz, 1H), 4.20-4.13 (m, 2H), 3.71-3.62 (m, 34H), 3.59 (s, 4H), 3.55 (d, J=5.1 Hz, 2H), 2.45 (s, 3H).

### 2. Preparation of Compound 3

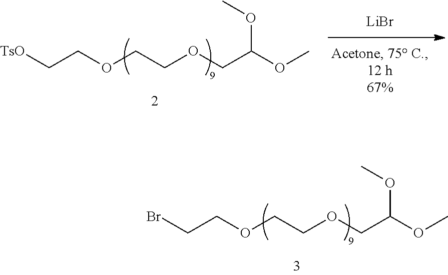

To a solution of 2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl 4-methylbenzenesulfonate (1.2 g, 1.71 mmol, 1.0 equiv) in acetone (8 mL) was added LiBr (744 mg, 8.56 mmol, 5.0 equiv), and then it was stirred at 75° C. for 12 h. The reaction mixture was poured into 50 mL water and extracted with EtOAc (3×20 mL). The organic layers were dried over anhydrous Na₂SO₄, filtered and concentrated to afford 2-[2-[2-[2-[2-[2-[2-[2-[2-[2-(2-bromoethoxy)ethoxy]ethoxy]ethoxy]ethoxy]ethoxy]ethoxy]ethoxy]ethoxy]ethoxy]-1,1-dimethoxy-ethane (700 mg, 1.15 mmol, 67% yield) as a yellow oil. ¹H NMR (400 MHz, CDCl3) δ 4.52 (t, J=5.2 Hz, 1H), 3.82 (t, J=6.3 Hz, 2H), 3.71-3.65 (m, 36H), 3.55 (d, J=5.3 Hz, 2H), 3.48 (t, J=6.4 Hz, 2H), 3.40 (s, 6H).

### 3. Preparation of Compound 5

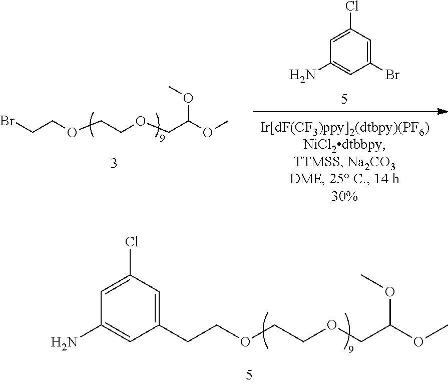

To a solution of 3-bromo-5-chloro-aniline (166.28 mg, 805.38 μmol, 1 eq) and 2-[2-[2-[2-[2-[2-[2-[2-[2-[2-(2-bromoethoxy)ethoxy]ethoxy]ethoxy]ethoxy]ethoxy]ethoxy]ethoxy]ethoxy]ethoxy]-1,1-dimethoxy-ethane (540 mg, 886 μmol, 1.1 equiv) in DME (12 mL) was added Ir[dF(CF₃)ppy]₂(dtbpy)(PF₆) (9 mg, 8 μmol, 0.01 equiv), NiCl₂·dtbbpy (1.6 mg, 4.0 μmol, 0.005 equiv), Na₂CO₃ (171 mg, 1.61 mmol, 2.0 equiv) and TTMSS (200 mg, 805 μmol, 1.0 equiv), and then it was stirred and irradiated with a 34 W blue LED lamp (7 cm away), with cooling fan to keep the reaction temperature at 25° C. for 14 h. The reaction mixture was filtered, and the filtrate was concentrated to afford crude product. The residue was purified by prep-HPLC (column: Waters Xbridge C18 150*50 mm*10 μm; mobile phase: [water (10 mM NH₄HCO₃)-ACN]; B %: 25%-55%) to afford 3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl]aniline (160 mg, 244 μmol, 30% yield) as a colorless oil. ¹H NMR (400 MHz, CDCl3) δ 6.61 (s, 1H), 6.56 (s, 1H), 6.51 (s, 1H), 4.52 (t, J=5.3 Hz, 1H), 3.70-3.60 (m, 38H), 3.55 (d, J=5.1 Hz, 2H), 3.40 (s, 6H), 2.77 (t, J=6.9 Hz, 2H).

### 4. Preparation of Compound 7

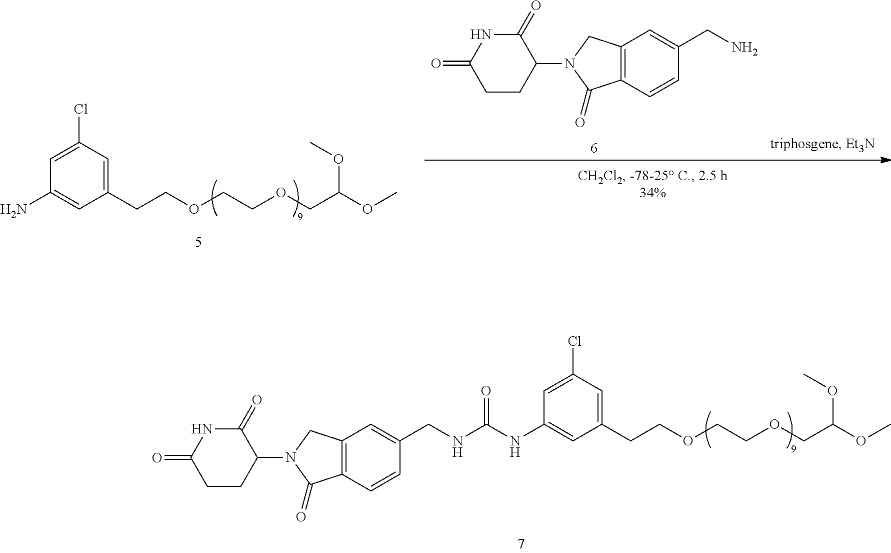

To a solution of triphosgene (36 mg, 0.12 mmol, 0.5 equiv) and Et₃N (74 mg, 0.73 mmol, 0.10 mL, 3.0 equiv) in CH₂Cl₂ (5 mL) was added 3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl]aniline (160 mg, 244 μmol, 1.0 equiv) in CH₂Cl₂ (2 mL) at −78° C., and then it was stirred for 0.5 h. 3-[5-(aminomethyl)-1-oxo-isoindolin-2-yl]piperidine-2,6-dione (91 mg, 0.29 mmol, 1.2 equiv, HCl salt) was added to the mixture, then it was slowly warmed to 25° C. and stirred for 2 h. The reaction mixture was poured into 50 mL sat. NaHCO₃, and then it was extracted with EtOAc (2×50 mL). The organic layers were dried over anhydrous Na₂SO₄, filtered and concentrated to afford crude product. The residue was purified by prep-TLC (CH₂Cl₂:MeOH=10:1) to afford 1-[3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (80 mg, 84 μmol, 34% yield) as a colorless oil. LC-MS: MS (ES⁺): m/z=972.2 [M+H₂O]⁺.

### 5. Preparation of Compound 8

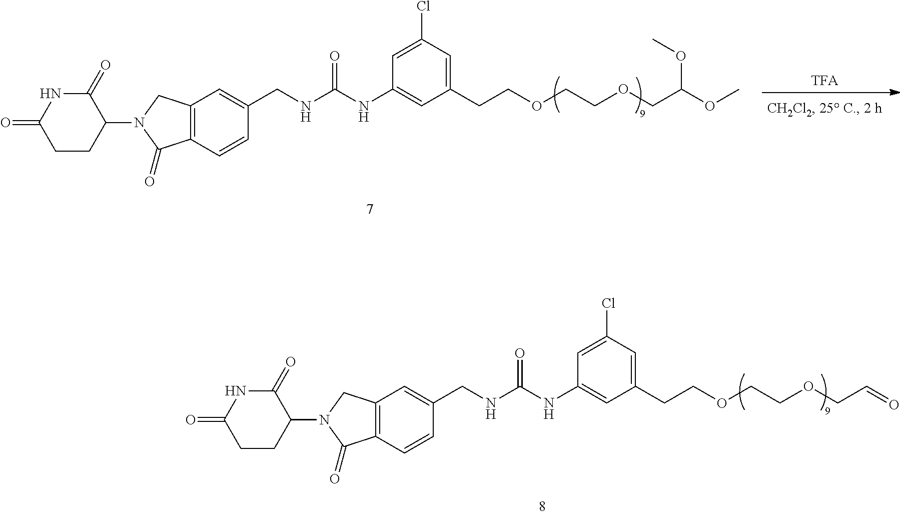

To a solution of 1-[3-chloro-5-[2-[2-[2-[2-[2-[2-[2-[2-[2-[2-(2,2-dimethoxyethoxy)ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (80 mg, 84 μmol, 1.0 equiv) in CH₂Cl₂ (2 mL) was added TFA (0.2 mL) and H₂O (0.04 mL), and then it was stirred at 25° C. for 2 h. The pH was adjusted to 7-8 by Et₃N at 0° C. 1-[3-chloro-5-[2-[2-[2-[2-[2-[2-[2-[2-[2-[2-(2- oxoethoxy) ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (76 mg, 84 μmol, 100% yield) was obtained as a yellow liquid in CH₂Cl₂. LC-MS: MS (ES⁺): m/z=909.6 [M+H⁺].

### 6. Preparation of I-12

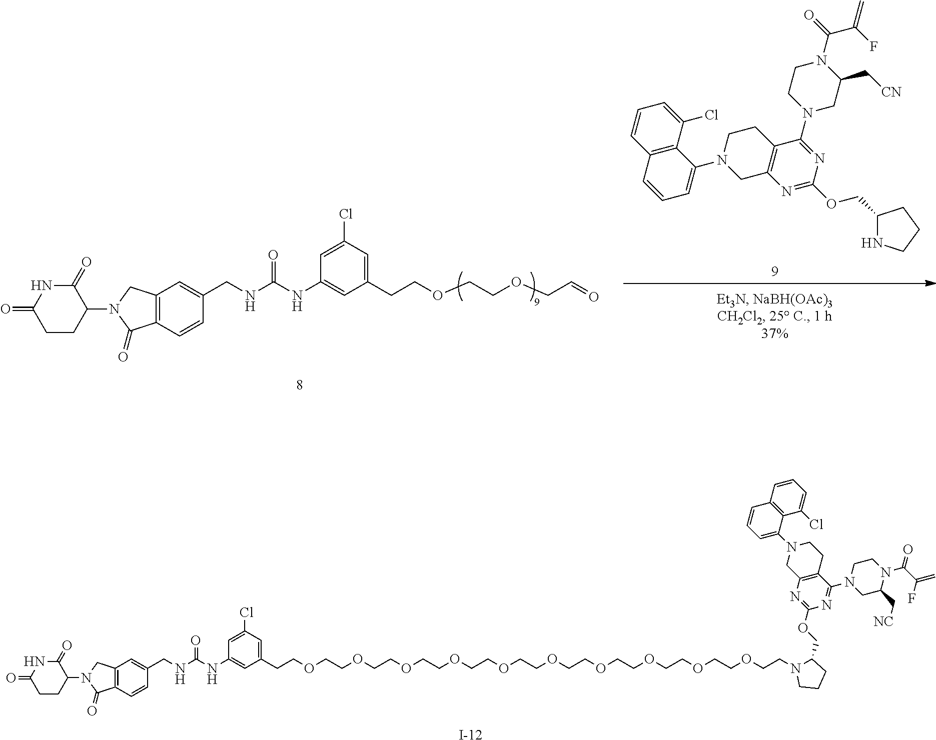

To a solution of 1-[3-chloro-5-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (76 mg, 84 μmol, 1.0 equiv) and 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (59 mg, 84 μmol, 1.0 equiv, TFA salt) in CH₂Cl₂ (2 mL) was added Et₃N (25 mg, 0.25 mmol, 35 μL, 3.0 equiv) and NaBH(OAc)₃ (177 mg, 836 μmol, 10.0 equiv), and then it was stirred at 25° C. for 1 h. The reaction mixture was poured into 20 mL water and extracted with CH₂Cl₂ (2×20 mL). The organic layers were washed with brine (2×20 mL), dried over anhydrous Na₂SO₄, filtered and concentrated to afford crude product. The residue was purified by prep-HPLC (column: Waters Xbridge 150*25 mm*5 μm; mobile phase: [water (10 mM NH₄HCO₃)-ACN]; B %: 45%-75%, 9 min) to afford 1-[3-chloro-5-[2-[2-[2-[2-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (46 mg, 31 μmol, 37% yield) as a light yellow solid. ¹H NMR (400 MHz, CD₃OD) δ 7.83 (s, 1H), 7.76 (d, J=7.7 Hz, 1H), 7.72-7.64 (m, 1H), 7.58-7.44 (m, 5H), 7.42-7.28 (m, 2H), 7.11 (s, 1H), 6.87 (s, 1H), 5.41-5.23 (m, 2H), 5.19-5.09 (m, 1H), 4.50 (s, 2H), 4.46 (d, J=7.1 Hz, 2H), 4.42-4.00 (m, 5H), 3.76-3.37 (m, 44H), 3.24-2.84 (m, 11H), 2.82-2.34 (m, 7H), 2.21-1.94 (m, 2H), 1.89-1.65 (m, 3H). LC-MS: MS (ES⁺): RT=2.753 min, m/z=742.6 [M/2+H⁺], LCMS method: LC-MS METHOD 25.M

### Example 13—Preparation of Compound I-13

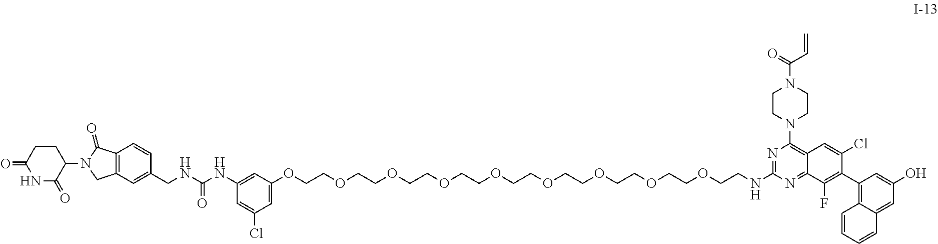 [SMILES: C=CC(=O)N1CCN(c2nc(NCCOCCOCCOCCOCCOCCOCCOCCOCCOc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

The title compound was prepared according to the following procedures.

### 1. Preparation of Compound 2

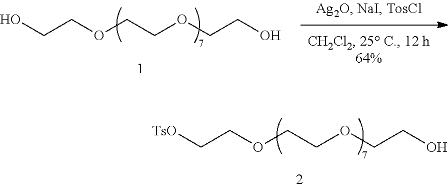

To a solution of 2-[2-[2-[2-[2-[2-[2-[2-(2-hydroxyethoxy)ethoxy]ethoxy]ethoxy]ethoxy]ethoxy]ethoxy]ethoxy]ethanol (10 g, 24 mmol, 1.0 equiv) in CH₂Cl₂ (150 mL) was added Ag₂O (8.39 g, 36.2 mmol, 1.50 equiv), NaI (3.98 g, 26.5 mmol, 1.1 equiv) and TosCl (4.6 g, 24 mmol, 1.0 equiv), and then it was stirred at 25° C. for 12 h. The reaction mixture was filtered and the filtrate was concentrated to afford crude product. The residue was purified by silica chromatography (EtOAc:MeOH=1:0-20:1) to afford 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (8.80 g, 15.5 mmol, 64% yield) as a colorless oil. ¹H NMR (400 MHz, CDCl₃) δ 7.81 (d, J=8.3 Hz, 2H), 7.35 (d, J=8.0 Hz, 2H), 4.23-4.13 (m, 2H), 3.78-3.52 (m, 34H), 2.46 (s, 3H). LC-MS: MS (ES⁺): m/z=569.5 [M+H⁺].

### 2. Preparation of Compound 3

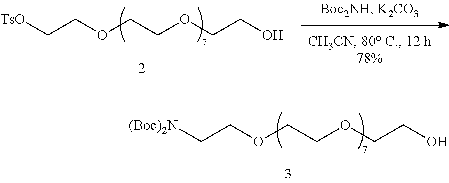

To a solution of 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (4.4 g, 7.7 mmol, 1.0 equiv) in CH₃CN (40 mL) was added K₂CO₃ (3.2 g, 23 mmol, 3.0 equiv) and tert-butyl N-tert-butoxycarbonylcarbamate (3.4 g, 16 mmol, 2.0 equiv), and then it was stirred at 80° C. for 12 h. The reaction mixture was filtered and the filtrate was concentrated to afford crude product. The residue was purified by silica chromatography (Petroleum ether/Ethyl acetate=1:1-0:1) to afford tert-butyl N-tert-butoxycarbonyl-N-[2-[2-[2-[2-[2-[2-[2-[2-(2-hydroxyethoxy)ethoxy]ethoxy]ethoxy]ethoxy]ethoxy]ethoxy]ethoxy]ethyl]carbamate (3.7 g, 6.0 mmol, 77% yield) as a colorless oil. ¹H NMR (400 MHz, CDCl₃) δ 3.82-3.77 (m, 2H), 3.78-3.73 (m, 2H), 3.71-3.56 (m, 32H), 1.51 (s, 18H).

### 3. Preparation of Compound 4

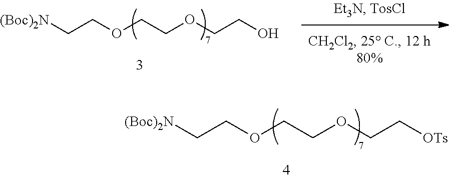

To a solution of tert-butyl N-tert-butoxycarbonyl-N-[2-[2-[2-[2-[2-[2-[2-[2-(2-hydroxyethoxy) ethoxy]ethoxy]ethoxy]ethoxy]ethoxy]ethoxy]ethoxy]ethyl]carbamate (3.7 g, 6.0 mmol, 1.0 equiv) in CH₂Cl₂ (20 mL) was added Et₃N (1.22 g, 12.1 mmol, 1.70 mL, 2.0 equiv) and TosCl (1.72 g, 9.04 mmol, 1.5 equiv), and then it was stirred at 25° C. for 12 h. The reaction mixture was concentrated to afford crude product. The residue was purified by silica chromatography (Petroleum ether/Ethyl acetate=1:1-0:1) to afford 2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl 4-methylbenzenesulfonate (3.7 g, 4.8 mmol, 80% yield) as a colorless oil. ¹H NMR: (400 MHz, CDCl₃) δ 7.81 (d, J=8.3 Hz, 2H), 7.35 (d, J=8.0 Hz, 2H), 4.20-4.13 (m, 2H), 3.83-3.77 (m, 2H), 3.74-3.53 (m, 32H), 2.46 (s, 3H), 1.50 (s, 18H).

### 4. Preparation of Compound 6

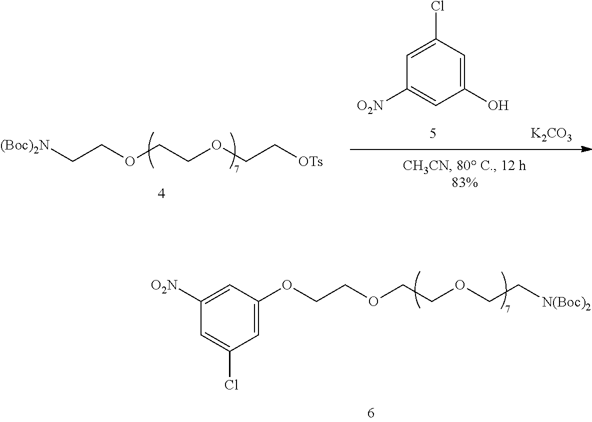

To a solution of 2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl 4-methylbenzenesulfonate (700 mg, 912 μmol, 1.0 equiv) and 3-chloro-5-nitro-phenol (158 mg, 912 μmol, 1.0 equiv) in CH₃CN (10 mL) was added K₂CO₃ (252 mg, 1.82 mmol, 2.0 equiv), and then it was stirred at 80° C. for 12 h. The reaction mixture was filtered, and the filtrate was concentrated to afford crude product. The residue was purified by silica chromatography (Petroleum ether/Ethyl acetate=1:1-0:1) to afford tert-butyl N-tert-butoxycarbonyl-N-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethyl]carbamate (580 mg, 754 μmol, 83% yield) as a colorless oil. LC-MS: MS (ES⁺): RT=0.994 min, m/z=786.1 [M+H₂O]⁺.

### 5. Preparation of Compound 7

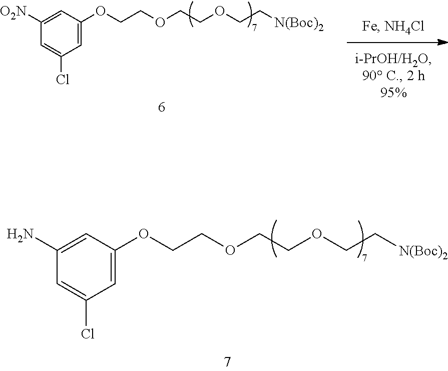

To a solution of tert-butyl N-tert-butoxycarbonyl-N-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethyl]carbamate (530 mg, 689 μmol, 1.0 equiv) in i-PrOH (27 mL) was added NH₄Cl (369 mg, 6.89 mmol, 10.0 equiv) in H₂O (3 mL), and then Fe (192 mg, 3.44 mmol, 5.0 equiv) was added. The reaction mixture was stirred at 90° C. for 2 h. The reaction mixture was diluted with EtOAc (50 mL), and then it was filtered. The filtrated was washed with water (2×50 mL), brine (2×20 mL), and then it was dried over anhydrous Na₂SO₄, filtered and concentrated to afford tert-butyl N-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethyl]-N-tert-butoxycarbonyl-carbamate (484 mg, 655 μmol, 95% yield) as a yellow oil and used for the next step directly. LC-MS: MS (ES⁺): RT=0.743 min, m/z=739.3 [M+H⁺].

### 6. Preparation of Compound 9

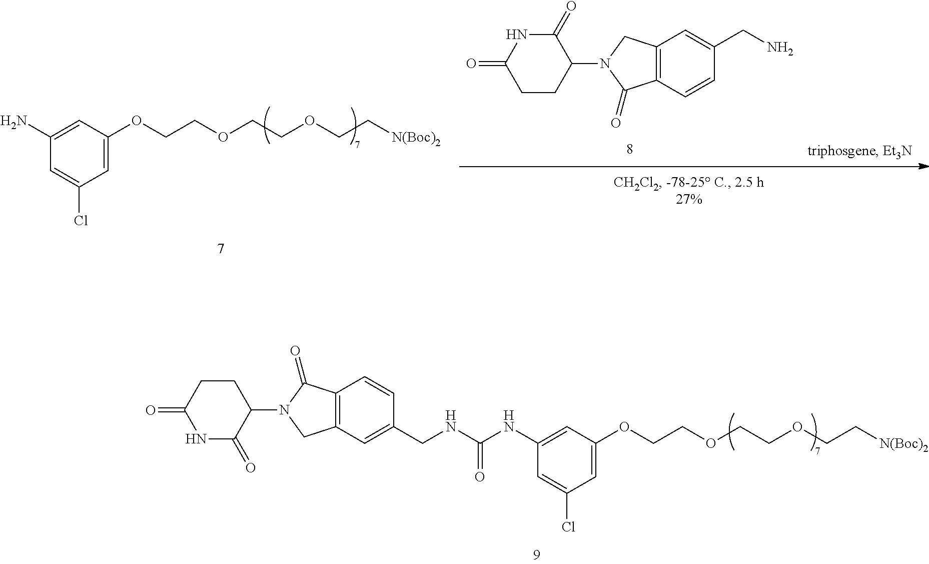

To a solution of TEA (197 mg, 1.95 mmol, 270 μL, 3.0 equiv) and triphosgene (96 mg, 33 μmol, 0.5 equiv) in CH₂Cl₂ (5 mL) was added tert-butyl N-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethyl]-N-tert-butoxycarbonyl-carbamate (0.48 g, 0.65 mmol, 1.0 equiv) in CH₂Cl₂ (5 mL) at −78° C., and then it was stirred for 0.5 h. 3-[5-(aminomethyl)-1-oxo-isoindolin-2-yl]piperidine-2,6-dione (241 mg, 779 μmol, 1.2 equiv, HCl salt) was added. The reaction mixture was slowly warmed to 25° C. and stirred for 2 h. The reaction mixture was poured into 50 mL sat. NaHCO₃, and then it was extracted with CH₂Cl₂ (2×50 mL). The organic layers were dried over anhydrous Na₂SO₄, filtered and concentrated to afford crude product. The residue was purified by silica chromatography (CH₂Cl₂:MeOH=50:1-30:1) to afford tert-butyl N-tert-butoxycarbonyl-N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethyl]carbamate (180 mg, 173 μmol, 27% yield) as a yellow oil. LC-MS: MS (ES⁺): m/z=1056.2 [M+H₂O]⁺.

### 7. Preparation of Compound 10

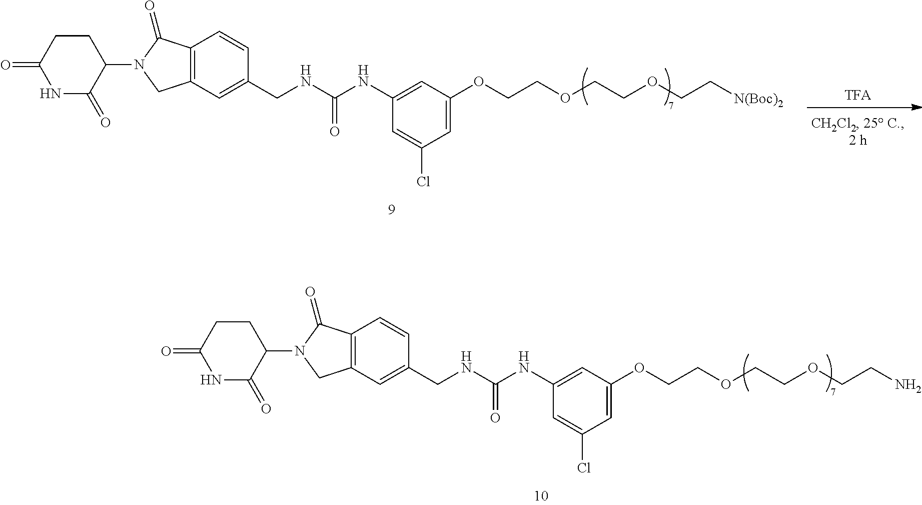

To a solution of tert-butyl N-tert-butoxycarbonyl-N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethyl]carbamate (180 mg, 173 μmol, 1.0 equiv) in CH₂Cl₂ (1 mL) was added TFA (0.5 mL), and then it was stirred at 25° C. for 2 h. The reaction mixture was concentrated to afford 1-[3-[2-[2-[2-[2-[2-[2-[2-[2-(2-aminoethoxy)ethoxy]ethoxy]ethoxy]ethoxy]ethoxy]ethoxy]ethoxy]ethoxy]-5-chloro-phenyl]-3-[[2-(2,6-dioxo-3-piperidyl)-1-oxo-isoindolin-5-yl]methyl]urea (165 mg, 173 μmol, 100% yield, TFA salt) as a yellow oil and used for the next step directly. LC-MS: MS (ES⁺): m/z=838.6 [M+H⁺].

### 8. Preparation of Compound 12

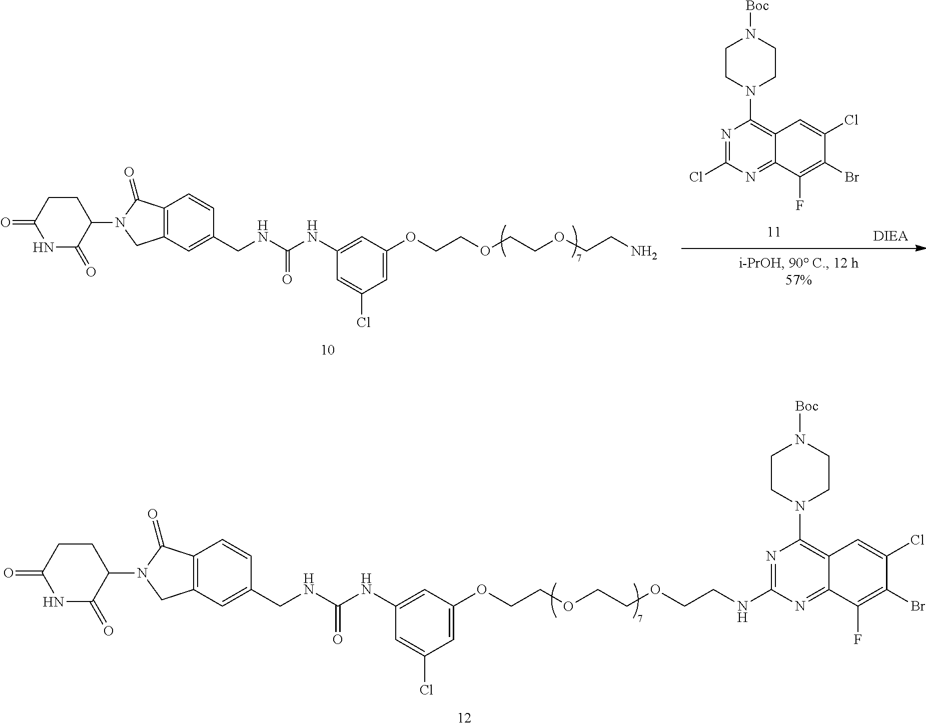

To a solution of 1-[3-[2-[2-[2-[2-[2-[2-[2-[2-(2-aminoethoxy)ethoxy]ethoxy]ethoxy]ethoxy]ethoxy]ethoxy]ethoxy]ethoxy]-5-chloro-phenyl]-3-[[2-(2,6-dioxo-3-piperidyl)-1-oxo-isoindolin-5-yl]methyl]urea (165 mg, 173 μmol, 1.0 equiv, TFA salt) and tert-butyl 4-(7-bromo-2,6-dichloro-8-fluoro-quinazolin-4-yl)piperazine-1-carboxylate (83 mg, 0.17 mmol, 1.0 equiv) in i-PrOH (2 mL) was added DIEA (112 mg, 866 μmol, 151 μL, 5.0 equiv), and then it was stirred at 90° C. for 12 h. The reaction mixture was concentrated to afford crude product. The residue was purified by silica chromatography (CH₂Cl₂:MeOH=50:1-30:1) to afford tert-butyl 4-[7-bromo-6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (150 mg, 99.5 μmol, 57% yield, 85% purity) as a yellow oil. LC-MS: MS (ES⁺): m/z=1281.3 [M+H⁺].

### 9. Preparation of Compound 14

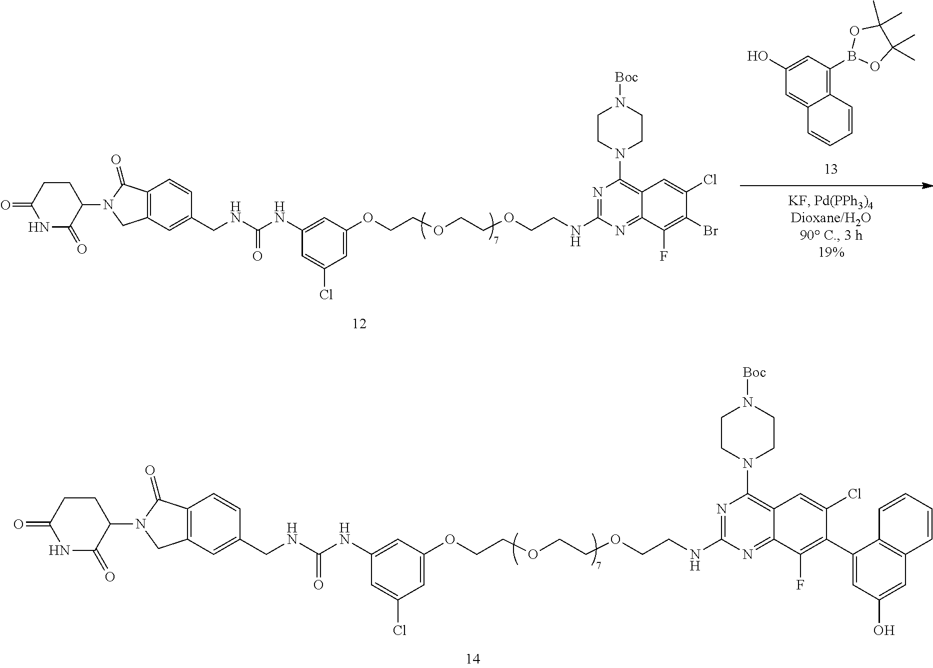

To a solution of tert-butyl 4-[7-bromo-6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (150 mg, 117 μmol, 1.0 equiv) and 4-(4,4,5,5-tetramethyl-1,3,2-dioxaborolan-2-yl)naphthalen-2-ol (48 mg, 0.18 μmol, 1.6 equiv) in dioxane (5 mL) was added Pd(PPh₃)₄ (27 mg, 23 μmol, 0.2 equiv) and KF (17 mg, 0.29 mmol, 2.5 equiv) in H₂O (1 mL), and then it was stirred at 90° C. for 3 h. The reaction mixture was poured into 20 mL water, and then it was extracted with EtOAc (2×20 mL). The organic layers were dried over anhydrous Na₂SO₄, filtered and concentrated to afford crude product. The residue was purified by prep-TLC (CH₂Cl₂:MeOH=10:1) to afford tert-butyl 4-[6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (30 mg, 22 μmol, 19% yield) as a yellow gum. LC-MS: MS (ES⁺): m/z=1343.3 [M+H⁺].

### 10. Preparation of Compound 15

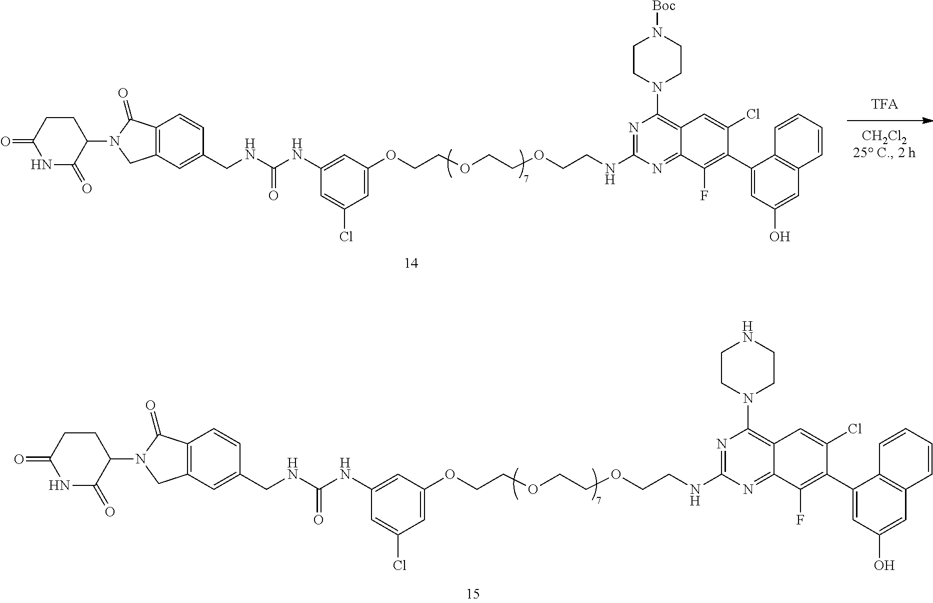

To a solution of tert-butyl 4-[6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethylamino]-8-fluoro-7-(3-hydroxy-1-naphthyl) quinazolin-4-yl]piperazine-1-carboxylate (30 mg, 22 μmol, 1.0 equiv) in CH₂Cl₂ (1 mL) was added TFA (0.5 mL), and then it was stirred at 25° C. for 2 h. The reaction mixture was concentrated to afford 1-[3-chloro-5-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (30 mg, 22 μmol, 99% yield, TFA salt) as a yellow oil and used for the next step directly. LC-MS: MS (ES⁺): m/z=1246.1 [M+H⁺].

### 11. Preparation of I-13

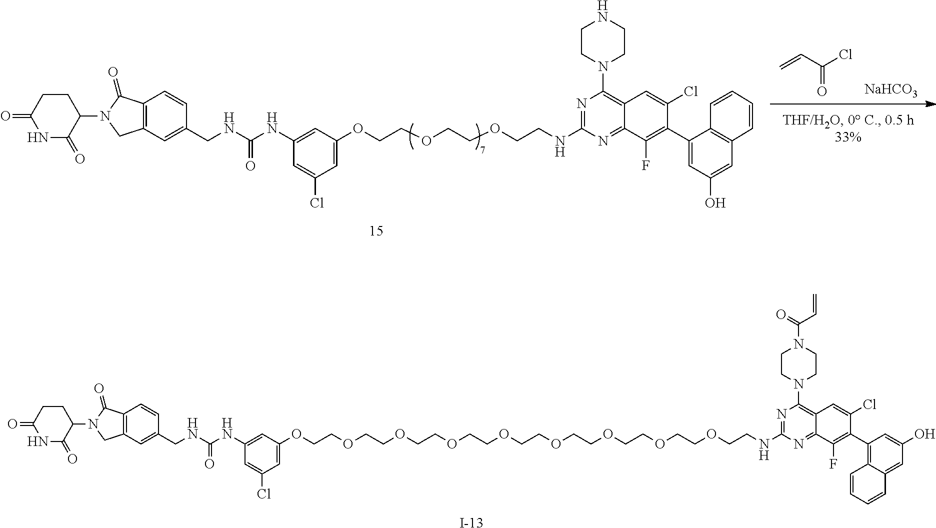

To a solution of 1-[3-chloro-5-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (30 mg, 22 μmol, 1.0 equiv, TFA salt) in THE (1 mL) was added NaHCO₃ (19 mg, 0.23 mmol, 10.0 equiv) in H₂O (0.5 mL), and then prop-2-enoyl chloride (2.0 mg, 22 μmol, 1.8 μL, 1.0 equiv) was added at 0° C. The resulting solution was stirred at 0° C. for 0.5 h. The reaction mixture was poured into 20 mL water and extracted with EtOAc (2×20 mL). The organic layers were dried over anhydrous Na₂SO₄, filtered and concentrated to afford crude product. The residue was purified by prep-HPLC (column: Phenomenex Gemini-NX C18 75*30 mm*3 μm; mobile phase: [water (0.225% FA)-ACN]; B %: 32%-42%, 7 min) to afford 1-[3-chloro-5-[2-[2-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]amino]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (9.5 mg, 7.3 μmol, 33% yield) as a white solid. ¹H NMR: (400 MHz, CD₃OD) δ 7.81 (s, 1H), 7.74 (d, J=7.9 Hz, 2H), 7.51 (s, 1H), 7.46 (d, J=8.0 Hz, 1H), 7.40 (t, J=7.3 Hz, 1H), 7.30-7.23 (m, 2H), 7.22-7.15 (m, 1H), 7.07-7.01 (m, 2H), 6.96 (t, J=1.9 Hz, 1H), 6.81 (dd, J=16.9 Hz, 1H), 6.54 (t, J=1.9 Hz, 1H), 6.27 (dd, J=16.8 Hz, 1H), 5.80 (dd, 10.6 Hz, 1H), 5.11 (dd, J=13.1 Hz, 1H), 4.48 (s, 2H), 4.41 (d, J=4.1 Hz, 2H), 4.09-4.02 (m, 2H), 3.94-3.74 (m, 10H), 3.70-3.54 (m, 32H), 2.93-2.70 (m, 2H), 2.49-2.35 (m, 1H), 2.18-2.05 (m, 1H). LC-MS: MS (ES⁺): RT=2.173 min, m/z=1298.4 [M+H⁺], LCMS method: LC-MS METHOD 25.

### Example 14—Preparation of Compound I-14

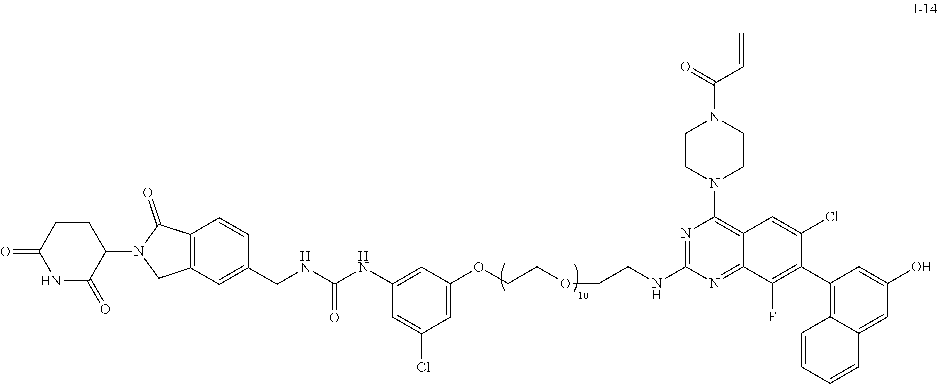

The title compound was prepared according to the following procedures.

### 1. Preparation of Compound 2

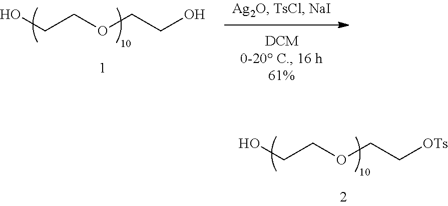

To a stirred solution of 2-[2-[2-[2-[2-[2-[2-[2-[2-[2-(2-hydroxyethoxy)ethoxy]ethoxy]ethoxy]-ethoxy]ethoxy]ethoxy]ethoxy]ethoxy]ethoxy]ethanol (20.0 g, 39.8 mmol, 1.0 equiv) in DCM (400 mL) were added Ag₂O (13.8 g, 59.7 mmol, 1.5 equiv), NaI (6.56 g, 43.8 mmol, 1.1 equiv) and TosCl (7.97 g, 41.8 mmol, 1.05 equiv) at 0° C. The reaction mixture was stirred at 20° C. for 16 h. The reaction mixture was filtered and the filtrate was concentrated in vacuo. The residue was purified by column chromatography on silica gel (Dichloromethane/Methanol=100/1 to 10/1) to afford 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl 4-methylbenzenesulfonate (16.0 g, 24.4 mmol, 61% yield) as a light yellow oil. ¹H NMR (400 MHz, CDCl₃): δ 7.84-7.75 (m, 2H), 7.40-7.30 (m, 2H), 4.18-4.12 (m, 2H), 3.76-3.55 (m, 42H), 2.44 (s, 3H).

### 2. Preparation of Compound 3

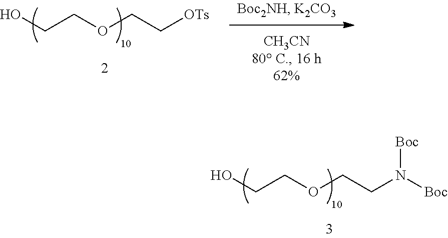

A mixture of 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl 4-methylbenzenesulfonate (16.0 g, 24.4 mmol, 1.0 equiv), tert-butyl N-tert-butoxycarbonylcarbamate (10.6 g, 48.7 mmol, 2.0 equiv), K₂CO₃ (10.1 g, 73.1 mmol, 3.0 equiv) in CH₃CN (160 mL) was stirred at 80° C. for 16 h under N₂. The reaction mixture was filtered and the filtrate was concentrated in vacuo. The residue was purified by column chromatography on silica gel (Dichloromethane/Methanol=100/1 to 50/1) to afford tert-butyl N-tert-butoxycarbonyl-N-[2-[2-[2-[2-[2-[2-[2-[2-[2-[2-(2-hydroxyethoxy) ethoxy]ethoxy]ethoxy]ethoxy]ethoxy]ethoxy]ethoxy]ethoxy]ethoxy]ethyl]carbamate (10.7 g, 15.2 mmol, 62% yield) as a colorless oil. ¹H NMR (400 MHz, CDCl₃): δ 3.81-3.76 (m, 2H), 3.75-3.71 (m, 2H), 3.70-3.58 (m, 41H), 1.50 (s, 18H).

### 3. Preparation of Compound 4

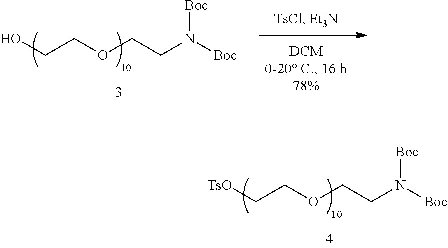

To a solution of tert-butyl N-tert-butoxycarbonyl-N-[2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl]carba mate (2.50 g, 3.56 mmol, 1.0 equiv) in DCM (50 mL) was added Et₃N (1.08 g, 10.7 mmol, 3.0 equiv) and TosCl (1.36 g, 7.12 mmol, 2.0 equiv) at 0° C. The reaction mixture was warmed to 20° C. and stirred for 16 h. The reaction mixture was concentrated in vacuo. The residue was purified by column chromatography on silica gel (Dichloromethane/Methanol=100/1 to 50/1) to afford 2-[2-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]-ethoxy]ethoxy]ethoxy]ethyl 4-methylbenzenesulfonate (2.38 g, 2.78 mmol, 78% yield) as a yellow oil. ¹H NMR (400 MHz, CDCl₃): δ 7.85-7.75 (m, 2H), 7.45-7.30 (m, 2H), 4.22-4.12 (m, 2H), 3.83-3.76 (m, 2H), 3.74-3.54 (m, 40H), 2.45 (s, 3H), 1.50 (s, 18H).

### 4. Preparation of Compound 6

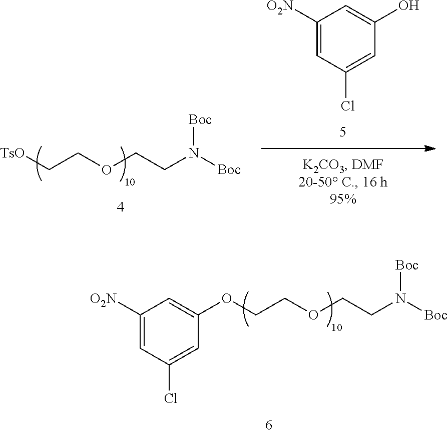

A mixture of 3-chloro-5-nitro-phenol (0.45 g, 2.59 mmol, 1.0 equiv), 2-[2-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethoxy]ethoxy]ethyl 4-methylbenzenesulfonate (2.38 g, 2.78 mmol, 1.07 equiv) and K₂CO₃ (0.72 g, 5.19 mmol, 2.0 equiv) in DMF (10 mL) was stirred at 50° C. for 16 h. The reaction mixture was diluted with water (30 mL) and the mixture was extracted with ethyl acetate (30 mL*3). The combined organic phase was washed with brine (30 mL), dried with anhydrous Na₂SO₄, filtered and concentrated in vacuo. The residue was purified by column chromatography on silica gel (Dichloromethane/Methanol=100/1 to 50/1) to afford tert-butyl N-tert-butoxycarbonyl-N-[2-[2-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]ethoxy]ethyl]carbamate (2.11 g, 2.46 mmol, 95% yield) as a yellow oil. ¹H NMR (400 MHz, CDCl₃): δ 7.84-7.79 (m, 1H), 7.70-7.66 (m, 1H), 7.27-7.24 (m, 1H), 4.24-4.18 (m, 2H), 3.92-3.85 (m, 2H), 3.82-3.76 (m, 2H), 3.74-3.70 (m, 2H), 3.70-3.67 (m, 2H), 3.66-3.58 (m, 34H), 1.50 (s, 18H).

### 5. Preparation of Compound 7

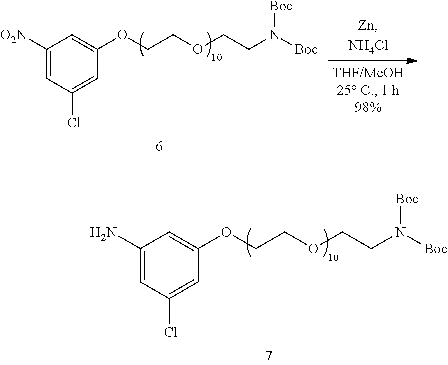

A mixture of tert-butyl N-tert-butoxycarbonyl-N-[2-[2-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]ethoxy]ethyl]carbamate (1.91 g, 2.23 mmol, 1.0 equiv), Zn (0.73 g, 11.1 mmol, 5.0 equiv) and NH₄Cl (0.60 g, 11.1 mmol, 5.0 equiv) in THF (15 mL) and MeOH (15 mL) was stirred at 25° C. for 1 h. The reaction mixture was filtered and the filtrate was concentrated in vacuo. The residue was dissolved in ethyl acetate (60 mL). The mixture was washed with water (30 mL), brine (30 mL), dried with anhydrous Na₂SO₄, filtered and concentrated in vacuo to afford tert-butyl N-[2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]ethoxy]ethyl]-N-tert-butoxycarbonyl-carbamate (1.84 g, 2.18 mmol, 98% yield) as a yellow oil. ¹H NMR (400 MHz, CDCl₃): δ 6.32-6.29 (m, 1H), 6.29-6.26 (m, 1H), 6.16-6.12 (m, 1H), 4.09-4.03 (m, 2H), 3.85-3.75 (m, 4H), 3.73-3.53 (m, 38H), 1.50 (s, 18H). LC-MS: MS (ES⁺): RT=0.749 min, m/z=827.2 [M+H⁺].

### 6. Preparation of Compound 9

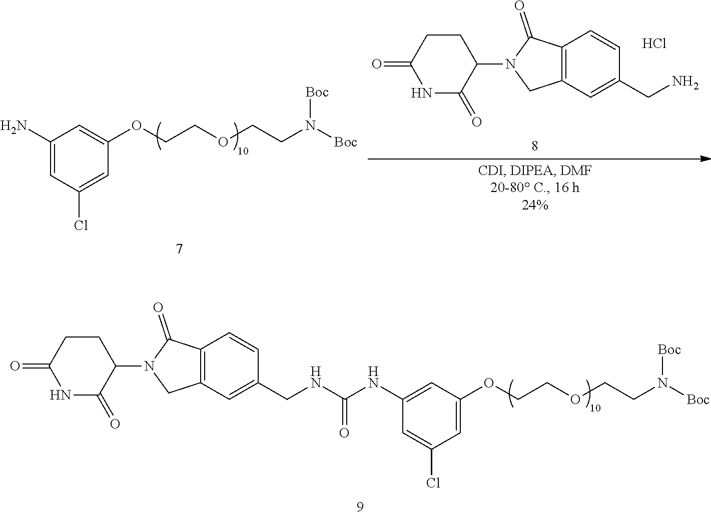

To a solution of 3-[5-(aminomethyl)-1-oxo-isoindolin-2-yl]piperidine-2,6-dione (0.69 g, 2.22 mmol, 1.0 equiv, HCl salt) in DMF (20 mL) were added CDI (0.54 g, 3.34 mmol, 1.5 equiv) and DIPEA (1.44 g, 11.1 mmol, 5.0 equiv) at 20° C. The reaction mixture was stirred at 20° C. for 1 h. To the reaction mixture was added tert-butyl N-[2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]ethoxy]ethyl]-N-tert-butoxycarbonyl-carbamate (1.84 g, 2.22 mmol, 1.0 equiv) and the mixture was stirred at 80° C. for 15 h. The reaction mixture was diluted with water (60 mL) and the mixture was extracted with ethyl acetate (50 mL*3). The combined organic phase was washed with brine (60 mL), dried with anhydrous Na₂SO₄, filtered and concentrated in vacuo. The residue was purified by column chromatography on silica gel (Dichloromethane/Methanol=100/1 to 10/1) to afford tert-butyl N-tert-butoxycarbonyl-N-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethyl]carbamate (0.61 g, 0.53 mmol, 24% yield) as a yellow oil. ¹H NMR (400 MHz, CDCl₃): δ 8.55-8.00 (m, 1H), 7.64-7.59 (m, 1H), 7.40 (s, 1H), 7.33-7.28 (m, 1H), 7.22-7.16 (m, 2H), 6.98-6.95 (m, 1H), 6.68-6.61 (m, 1H), 6.47-6.43 (m, 1H), 5.14-5.06 (m, 1H), 4.55-4.35 (m, 2H), 4.30-4.15 (m, 2H), 4.06-3.98 (m, 2H), 3.82-3.73 (m, 4H), 3.70-3.52 (m, 38H), 2.92-2.70 (m, 2H), 2.36-2.22 (m, 1H), 2.20-2.10 (m, 1H), 1.49 (s, 18H). LC-MS: MS (ES⁺): RT=0.738 min, m/z=1026.3 [M+H⁺].

### 7. Preparation of Compound 10

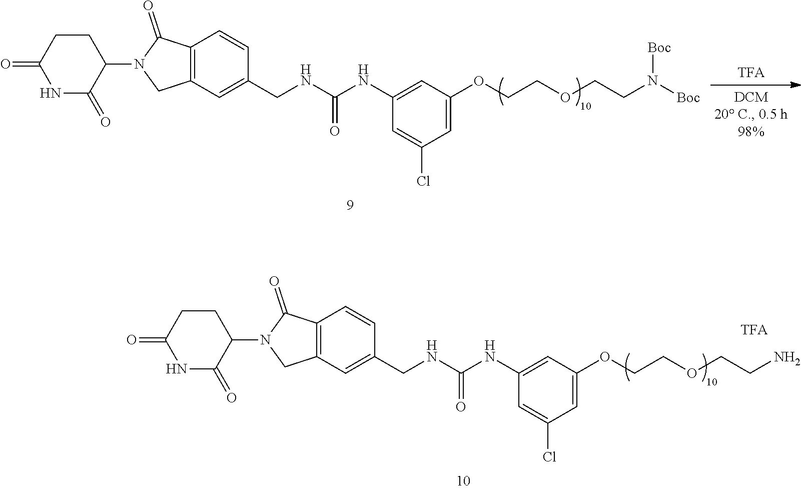

A mixture of tert-butyl N-tert-butoxycarbonyl-N-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethyl]carbamate (610 mg, 0.54 mmol, 1.0 equiv) in TFA (3 mL) and DCM (6 mL) was stirred at 20° C. for 0.5 h. The reaction mixture was concentrated in vacuo to afford 1-[3-[2-[2-[2-[2-[2-[2-[2-[2-[2-[2-(2-aminoethoxy) ethoxy]ethoxy]ethoxy]ethoxy]ethoxy]ethoxy]ethoxy]ethoxy]ethoxy]ethoxy]-5-chloro-phenyl]-3-[[2-(2,6-dioxo-3-piperidyl)-1-oxo-isoindolin-5-yl]methyl]urea (563 mg, 0.53 mmol, 98% yield, TFA salt) as a yellow oil. LC-MS: MS (ES⁺): m/z=926.7 [M+H⁺].

### 8. Preparation of Compound 12

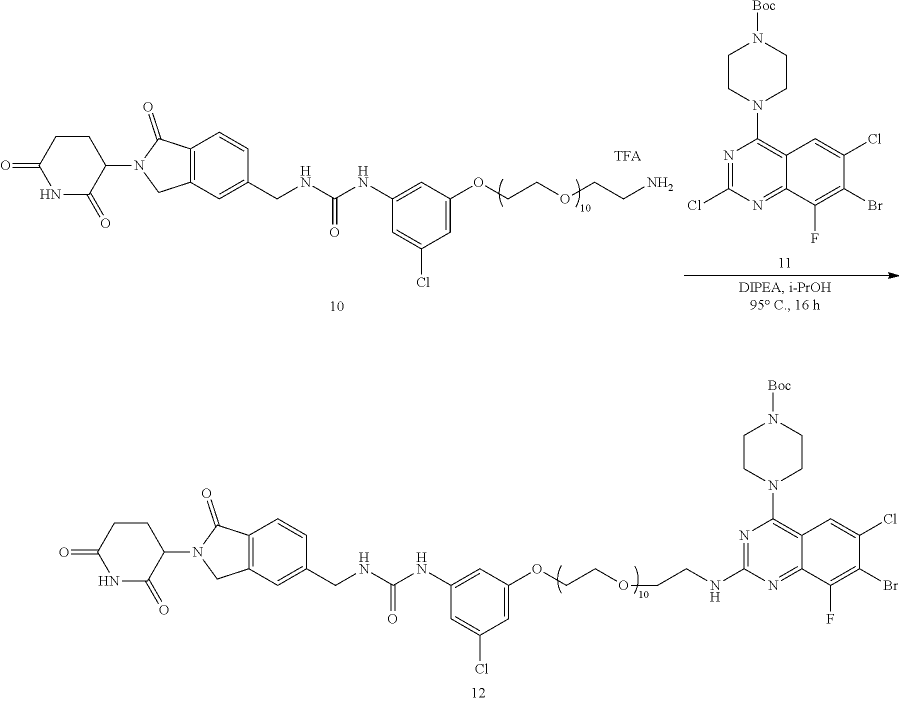

A mixture of tert-butyl 4-(7-bromo-2,6-dichloro-8-fluoro-quinazolin-4-yl)piperazine-1-carboxylate (260 mg, 0.54 mmol, 1.0 equiv), 1-[3-[2-[2-[2-[2-[2-[2-[2-[2-[2-[2-(2-aminoethoxy) ethoxy]ethoxy]ethoxy]ethoxy]ethoxy]ethoxy]ethoxy]ethoxy]ethoxy]ethoxy]-5-chloro-phenyl]-3-[[2-(2,6-dioxo-3-piperidyl)-1-oxo-isoindolin-5-yl]methyl]urea (563 mg, 0.54 mmol, 1.0 equiv, TFA salt) and DIPEA (350 mg, 2.71 mmol, 5.0 equiv) in i-PrOH (5 mL) in a sealed tube was stirred at 95° C. for 16 h. The reaction mixture was diluted with water (5 mL) and the mixture was extracted with ethyl acetate (5 mL*3). The combined organic phase was washed with brine (5 mL), dried with anhydrous Na₂SO₄, filtered and concentrated in vacuo. The residue was purified by column chromatography on silica gel (Dichloromethane/Methanol=100/1 to 6/1) and prep-HPLC (column: Waters Xbridge C18 150*50 mm*10 μm; mobile phase: [water (10 mM NH₄HCO₃)-ACN]; B %: 47%-77%, 2 min) to afford tert-butyl 4-[7-bromo-6-chloro-2-[2-[2-[2-[2-[2-[2-[2-[2-[2-[2-[2-[3-chloro-5-[[2- (2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (210 mg, 85% purity) as a yellow oil. LC-MS: MS (ES⁺): m/z=1370.6 [M+H⁺].

### 9. Preparation of Compound 14

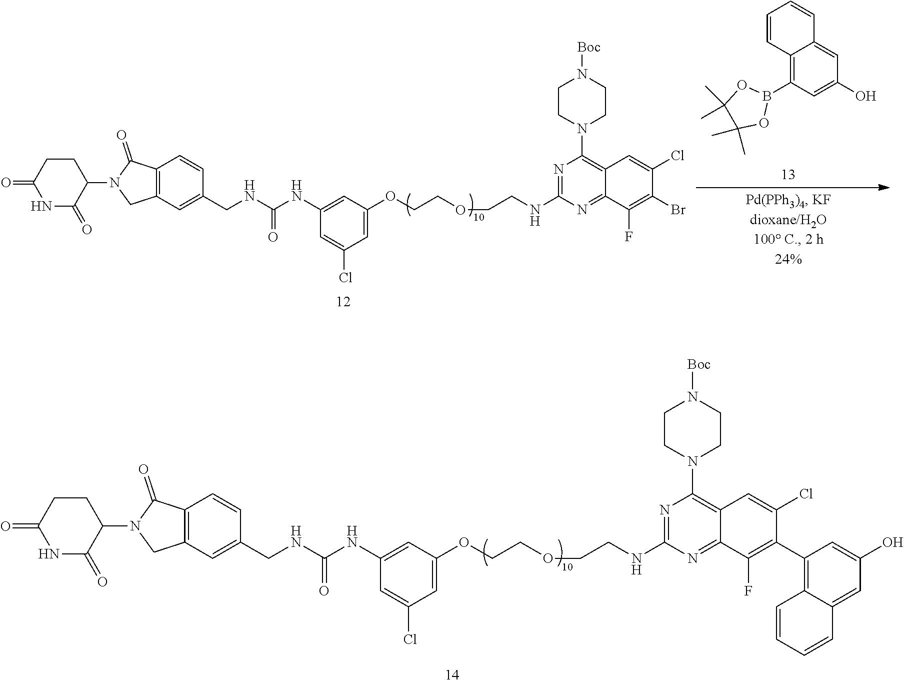

A mixture of tert-butyl 4-[7-bromo-6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (180 mg, 112 μmol, 85% purity, 1.0 equiv), 4-(4,4,5,5-tetramethyl-1,3,2-dioxaborolan-2-yl)naphthalen-2-ol (45.3 mg, 168 μmol, 1.5 equiv), Pd(PPh₃)₄ (64.5 mg, 55.8 μmol, 0.5 equiv) and KF (16.2 mg, 279 μmol, 2.5 equiv) in dioxane (18 mL) and H₂O (3.6 mL) was stirred at 100° C. for 2 h under N₂. The reaction mixture was diluted with water (50 mL) and the mixture was extracted with ethyl acetate (50 mL*3). The combined organic phase was washed with brine (50 mL), dried with anhydrous Na₂SO₄, filtered and concentrated in vacuo. The residue was purified by prep-TLC (Dichloromethane/Methanol=10/1) to afford tert-butyl 4-[6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (40.0 mg, 26.8 μmol, 24% yield) as a brown oil. LC-MS: MS (ES⁺): m/z=1434.9 [M+H⁺].

### 10. Preparation of Compound 15

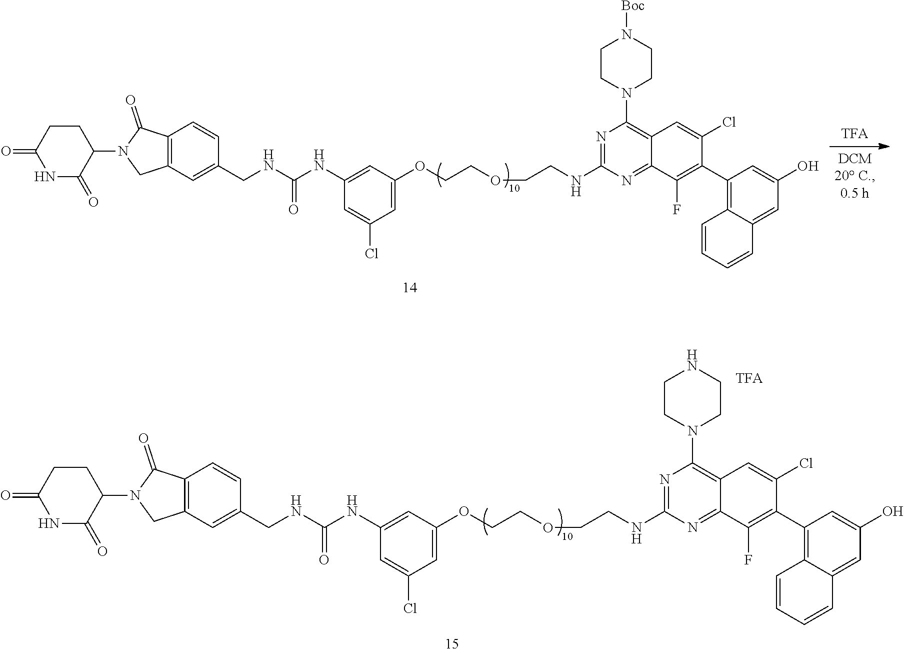

A mixture of tert-butyl 4-[6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethylamino]-8-fluoro-7-(3-hydroxy-1-naphthyl) quinazolin-4-yl]piperazine-1-carboxylate (30.0 mg, 20.9 μmol, 1.0 equiv) in DCM (3 mL) and TFA (1.5 mL) was stirred at 20° C. for 0.5 h. The reaction mixture was concentrated in vacuo to afford 1-[3-chloro-5-[2-[2-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (30.0 mg, crude, TFA salt) as a yellow oil. LC-MS: MS (ES⁺): m/z=1332.6 [M+H⁺].

### 11. Preparation of I-14

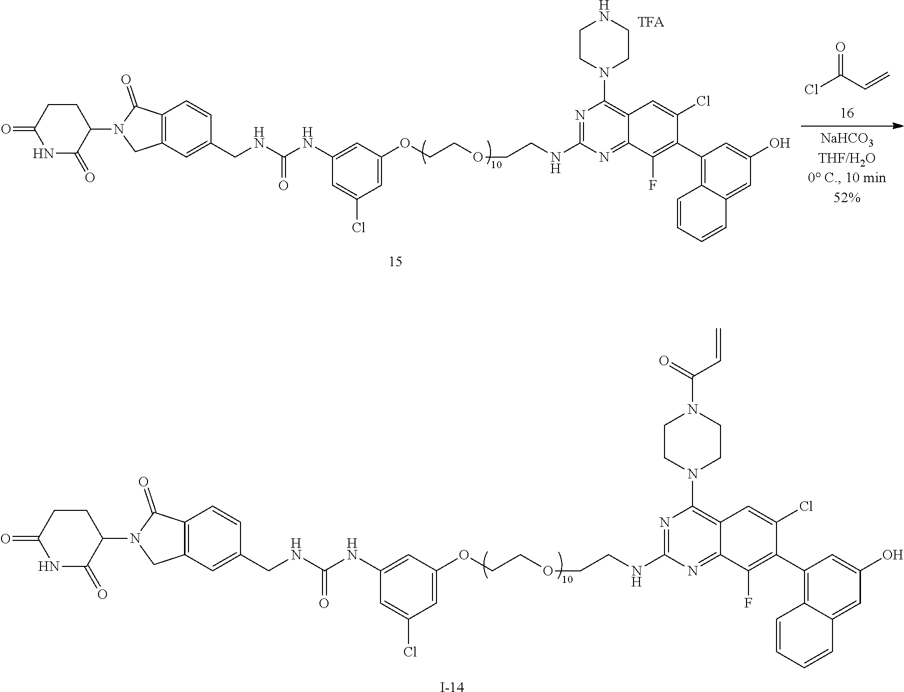

To a mixture of 1-[3-chloro-5-[2-[2-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (30.0 mg, 20.7 μmol, 1.0 equiv, TFA salt) and NaHCO₃ (17.4 mg, 207 μmol, 10.0 equiv) in THE (1.5 mL) and H₂O (0.3 mL) was added a solution of prop-2-enoyl chloride (1.88 mg, 20.7 μmol, 1.0 equiv) in THF (0.4 mL) at 0° C. The reaction mixture was diluted with water (5 mL) and the mixture was extracted with ethyl acetate (5 mL*3). The combined organic layer was washed with brine (5 mL), dried with anhydrous Na₂SO₄, filtered and concentrated in vacuo. The residue was purified by prep-HPLC (column: Phenomenex Luna C18 150*25 mm*10 μm; mobile phase: [water (0.225% FA)-ACN]; B %: 21%-51%, 10 min) to afford 1-[3-chloro-5-[2-[2-[2-[2-[2-[2-[2-[2-[2-[2-[2-[[6-chloro-8-fluoro-7-(3- hydroxy-1-naphthyl)-4-(4-prop-2-enoylpiperazin-1-yl)quinazolin-2-yl]amino]ethoxy]ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (15.3 mg, 10.9 μmol, 52% yield, 99% purity) as an off-white solid. ¹H NMR (400 MHz, DMSO-d₆): δ 10.97 (brs, 1H), 9.98 (brs, 1H), 8.89 (s, 1H), 7.82-7.77 (m, 2H), 7.71-7.66 (m, 1H), 7.50 (s, 1H), 7.46-7.40 (m, 2H), 7.28-7.25 (m, 1H), 7.24-7.20 (m, 2H), 7.17-7.14 (m, 1H), 7.06-7.02 (m, 1H), 6.96-6.93 (m, 1H), 6.93-6.88 (m, 1H), 6.88-6.80 (m, 1H), 6.57-6.54 (m, 1H), 6.21-6.13 (m, 1H), 5.77-5.71 (m, 1H), 5.14-5.06 (m, 1H), 4.48-4.38 (m, 3H), 4.34-4.27 (m, 1H), 4.06-4.01 (m, 2H), 3.86-3.74 (m, 6H), 3.73-3.68 (m, 3H), 3.59-3.44 (m, 42H), 2.96-2.85 (m, 1H), 2.64-2.55 (m, 1H), 2.41-2.34 (m, 1H), 2.03-1.95 (m, 1H). LC-MS: MS (ES⁺): RT=2.185 min, m/z=694.4 [1/2M+H⁺]; LCMS method: LC-MS METHOD 25.

### Example 15—Preparation of Compound I-15

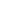

The title compound was prepared according to the following procedures.

### 1. Preparation of Compound 2

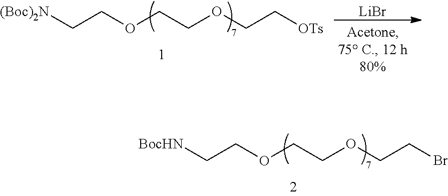

To a solution of 2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl 4-methylbenzenesulfonate (3.0 g, 3.9 mmol, 1.0 equiv) in acetone (20 mL) was added LiBr (1.70 g, 19.5 mmol, 5.0 equiv), and then it was stirred at 75° C. for 12 h. The reaction mixture was poured into 100 mL water and extracted with EtOAc (3×30 mL). The organic layers were washed with brine (2×20 mL), dried over anhydrous Na₂SO₄, filtered and concentrated to afford tert-butyl N-[2-[2-[2-[2-[2-[2-[2-[2-(2-bromoethoxy) ethoxy]ethoxy]ethoxy]ethoxy]ethoxy]ethoxy]ethoxy]ethyl]carbamate (1.8 g, 3.1 mmol, 80% yield) as a yellow oil and used for the next step directly. ¹H NMR: (400 MHz, CDCl₃) δ 3.82 (t, J=6.3 Hz, 2H), 3.72-3.58 (m, 28H), 3.55 (t, J=5.1 Hz, 2H), 3.48 (t, J=6.3 Hz, 2H), 3.32 (t, J=5.1 Hz, 2H), 1.45 (s, 9H).

### 2. Preparation of Compound 4

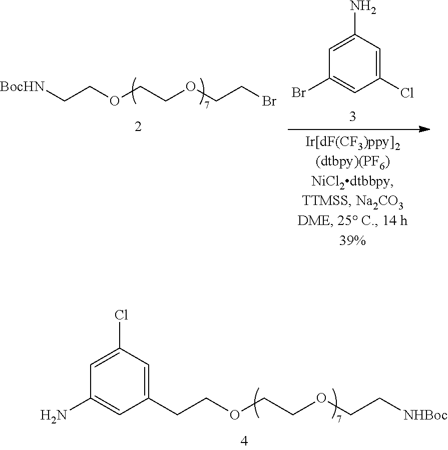

To a solution of 3-bromo-5-chloro-aniline (496 mg, 2.40 mmol, 1.00 equiv) and tert-butyl N-[2-[2-[2-[2-[2-[2-[2-[2-(2-bromoethoxy)ethoxy]ethoxy]ethoxy]ethoxy]ethoxy]ethoxy]ethoxy]ethyl]carbamate (1.8 g, 3.1 mmol, 1.3 equiv) in DME (24 mL) was added Ir[dF(CF₃)ppy]₂(dtbpy)(PF₆) (27 mg, 24 μmol, 0.01 equiv), NiCl₂·dtbbpy (4.8 mg, 12 μmol, 0.005 equiv), Na₂CO₃ (509 mg, 4.80 mmol, 2.0 equiv) and TTMSS (597 mg, 2.40 mmol, 741 μL, 1.0 equiv), and then it was stirred and irradiated with a 34 W blue LED lamp (7 cm away), with cooling fan to keep the reaction temperature at 25° C. for 14 h. The reaction mixture was filtered, and the filtrate was concentrated to afford crude product. The residue was purified by prep-HPLC (column: Waters Xbridge 150*25 mm*5 μm; mobile phase: [water (10 mM NH₄HCO₃)-ACN]; B %: 36%-66%, 9 min) to afford tert-butyl N-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethyl]carbamate (580 mg, 931 μmol, 39% yield) as a yellow oil. ¹H NMR (400 MHz, CDCl₃) δ 6.59 (s, 1H), 6.53 (s, 1H), 6.47 (s, 1H), 5.15-5.01 (m, 1H), 3.84-3.58 (m, 32H), 3.54 (t, J=5.0 Hz, 2H), 3.32 (d, J=5.3 Hz, 2H), 2.77 (t, J=6.9 Hz, 2H), 1.45 (s, 9H).

### 3. Preparation of Compound 6

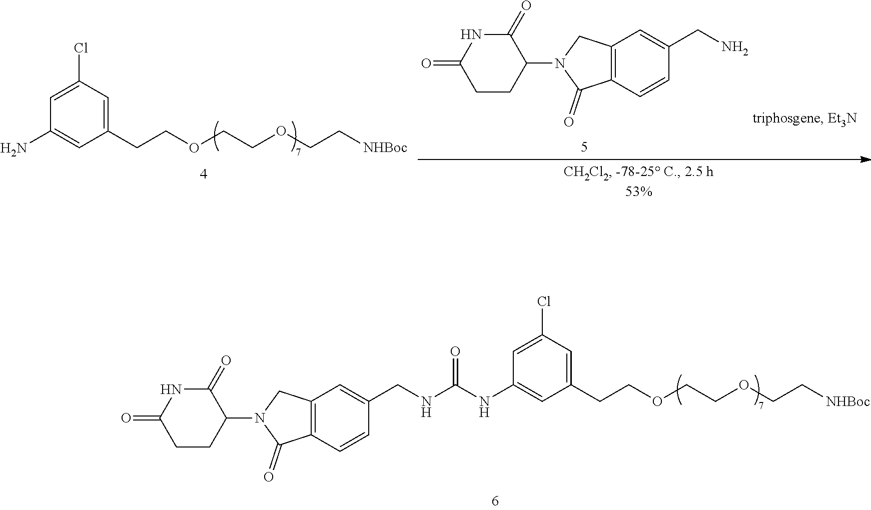

To a solution of triphosgene (90 mg, 0.30 mmol, 0.5 equiv) and Et₃N (185 mg, 1.83 mmol, 255 μL, 3.0 equiv) in CH₂Cl₂ (4 mL) was slowly added tert-butyl N-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethyl]carbamate (380 mg, 610 μmol, 1.0 equiv) in CH₂Cl₂ (6 mL) at −78° C., and then it was stirred for 0.5 h. 3-[5-(aminomethyl)-1-oxo-isoindolin-2-yl]piperidine-2,6-dione (200 mg, 732 μmol, 1.2 equiv) was added to the mixture, then it was slowly warmed to 25° C. and stirred for 2 h. The reaction mixture was poured into 50 mL sat. NaHCO₃, and then it was extracted with CH₂Cl₂ (2×50 mL). The organic layers were dried over anhydrous Na₂SO₄, filtered and concentrated to afford crude product. The residue was purified by silica chromatography (CH₂Cl₂:MeOH=50:1-30:1) to afford tert-butyl N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethyl]carbamate (300 mg, 325 μmol, 53% yield) as a yellow oil. LC-MS: MS (ES⁺): m/z=922.2 [M+H⁺].

### 4. Preparation of Compound 7

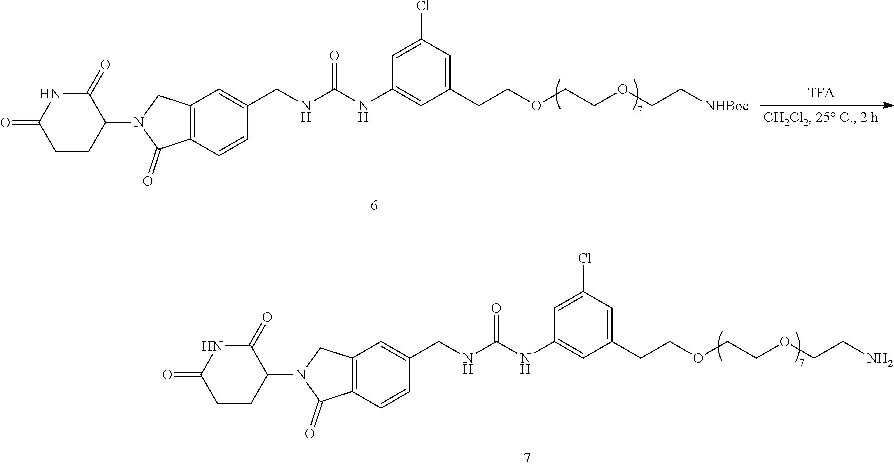

To a solution of tert-butyl N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethyl]carbamate (450 mg, 488 μmol, 1.0 equiv) in CH₂Cl₂ (2 mL) was added TFA (1 mL), and then it was stirred at 25° C. for 2 h. The reaction mixture was concentrated to afford crude product. 1-[3-[2-[2-[2-[2-[2-[2-[2-[2-(2-aminoethoxy)ethoxy]ethoxy]ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (455 mg, 486 μmol, 100% yield, TFA salt) was obtained as a yellow oil and used for the next step directly. LC-MS: MS (ES⁺): m/z=822.5 [M+H⁺].

### 5. Preparation of Compound 9

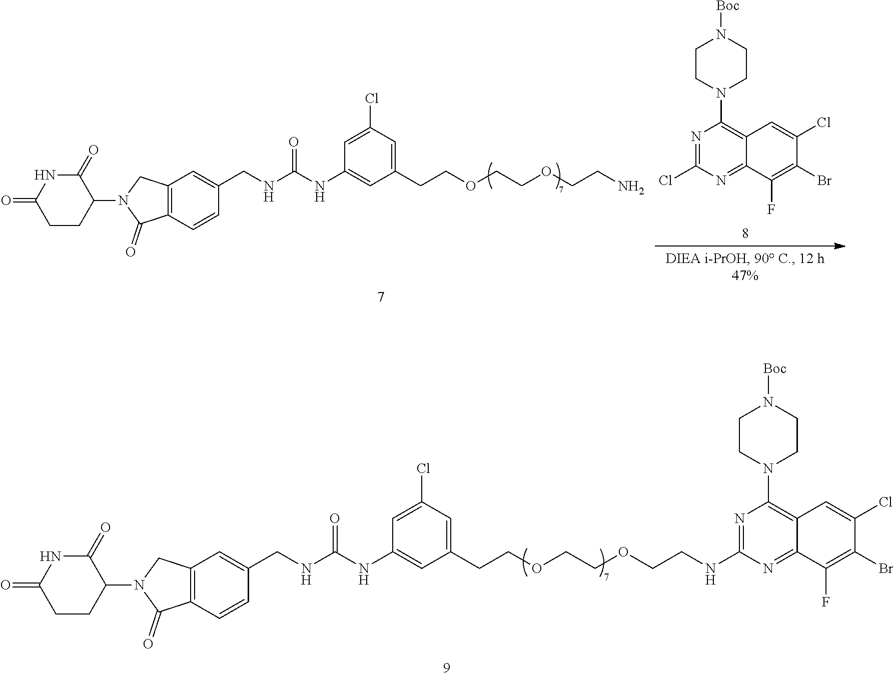

To a solution of 1-[3-[2-[2-[2-[2-[2-[2-[2-[2-(2-aminoethoxy)ethoxy]ethoxy]ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (455 mg, 486 μmol, 1.0 equiv, TFA salt) and tert-butyl 4-(7-bromo-2,6-dichloro-8-fluoro-quinazolin-4-yl)piperazine-1-carboxylate (233 mg, 486 μmol, 1.0 equiv) in i-PrOH (5 mL) was added DIEA (314 mg, 2.43 mmol, 423 μL, 5.0 equiv), and then it was stirred at 90° C. for 12 h. The reaction mixture was concentrated to afford crude product. The residue was purified by silica chromatography (CH₂Cl₂:MeOH=50:1-30:1) to afford tert-butyl 4-[7-bromo-6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethylamino]-8-fluoro-quinazolin-4-yl]piperazine-1-carboxylate (410 mg, 227 μmol, 47% yield, 70% purity) as a yellow oil. LC-MS: MS (ES⁺): m/z=1265.3 [M+H⁺].

### 6. Preparation of Compound 11

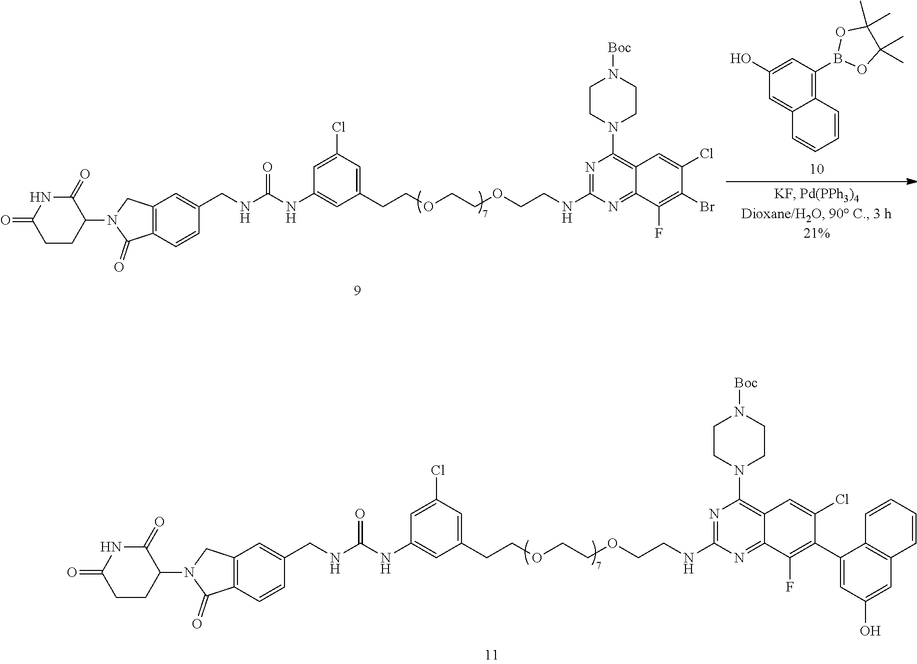

To a solution of tert-butyl 4-[7-bromo-6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethylamino]-8-fluoro-quinazolin-4-yl]piperazine-1-carboxylate (410 mg, 323 μmol, 1.0 equiv) and 4-(4,4,5,5-tetramethyl-1,3,2-dioxaborolan-2-yl)naphthalen-2-ol (131 mg, 486 μmol, 1.5 equiv) in dioxane (15 mL) was added Pd(PPh₃)₄ (75 mg, 65 μmol, 0.2 equiv) and KF (47 mg, 0.81 mmol, 2.5 equiv) in H₂O (3 mL), and then it was stirred at 90° C. for 3 h. The reaction mixture was poured into 50 mL and then it was extracted with EtOAc (2×20 mL). The organic layers were dried over anhydrous Na₂SO₄, filtered and concentrated to afford crude product. The residue was purified by silica chromatography (CH₂Cl₂:MeOH=50:1-30:1) and prep-TLC (CH₂Cl₂:MeOH=10:1) to afford tert-butyl 4-[6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (90 mg, 68 μmol, 21% yield) as a yellow gum. LC-MS: MS (ES⁺): m/z=1328.3 [M+H⁺].

### 7. Preparation of Compound 12

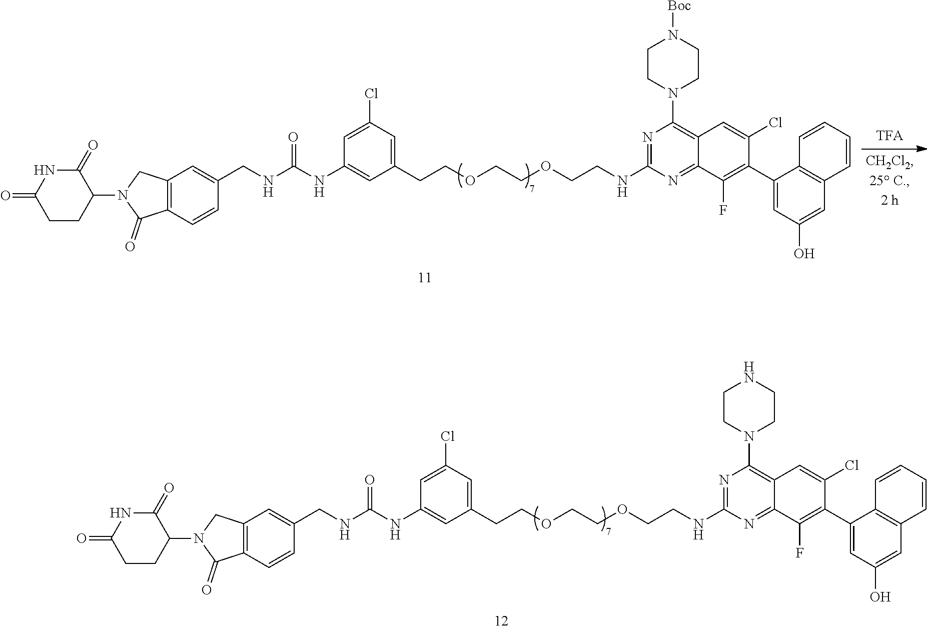

To a solution of tert-butyl 4-[6-chloro-2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (90 mg, 68 μmol, 1.0 equiv) in CH₂Cl₂ (1 mL) was added TFA (0.5 mL), and then it was stirred at 25° C. for 2 h. The reaction mixture was concentrated to afford crude product. 1-[3-chloro-5-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (91 mg, crude, TFA salt) was obtained as a yellow oil and used for the next step directly. LC-MS: MS (ES⁺): RT=0.828 min, m/z=1230.8 [M+H⁺].

### 8. Preparation of I-15

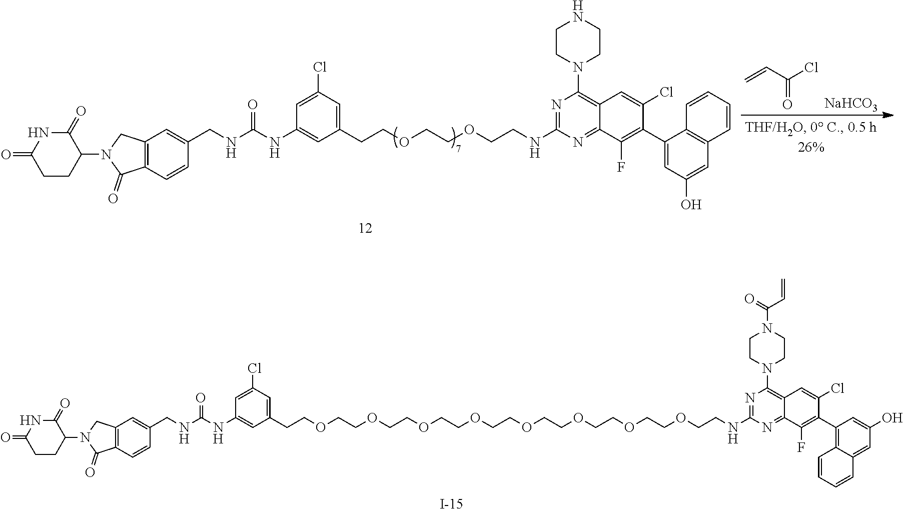

To a solution of 1-[3-chloro-5-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (91 mg, 68 μmol, 1.0 equiv, TFA salt) in THE (2 mL) was added NaHCO₃ (57 mg, 0.68 mmol, 10.0 equiv) in H₂O (1 mL), and then prop-2-enoyl chloride (6.13 mg, 67.8 μmol, 5.52 μL, 1.0 equiv) was added at 0° C. The resulting solution was stirred at 0° C. for 0.5 h. The reaction mixture was poured into 20 mL water and extracted with EtOAc (2×20 mL). The organic layers were dried over anhydrous Na₂SO₄, filtered and concentrated to afford crude product. The residue was purified by prep-HPLC (column: Phenomenex Gemini-NX C18 75*30 mm*3 μm; mobile phase: [water (0.225% FA)-ACN]; B %: 32%-42%, 7 min) to afford 1-[3-chloro-5-[2-[2-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]amino]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (23 mg, 17.92 μmol, 26% yield) as a white solid. ¹H NMR: (400 MHz, CD₃OD) δ 7.82 (s, 1H), 7.74 (d, J=7.8 Hz, 2H), 7.52 (s, 1H), 7.49-7.36 (m, 3H), 7.30-7.22 (m, 2H), 7.22-7.16 (m, 1H), 7.09 (s, 1H), 7.07-7.00 (m, 1H), 6.85 (d, J=1.6 Hz, 1H), 6.82-6.74 (m, 1H), 6.27 (dd, J=16.8 Hz, 1H), 5.83-5.76 (dd, J=8.4 Hz, 1H), 5.11 (dd, J=12.9 Hz, 1H), 4.49 (s, 2H), 4.42 (m, 2H), 4.10-3.76 (m, 10H), 3.73-3.53 (m, 36H), 2.93-2.69 (m, 4H), 2.49-2.35 (m, 1H), 2.20-2.06 (m, 1H). LC-MS: MS (ES⁺): RT=2.188 min, m/z=1282.6 [M+H⁺], LCMS method: LC-MS METHOD 25.

### Example 16—Preparation of Compound I-16

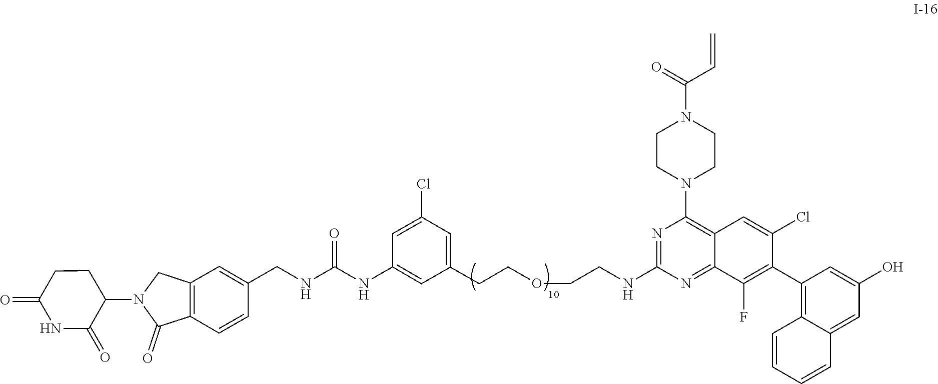

The title compound was prepared according to the following procedures.

### 1. Preparation of Compound 2

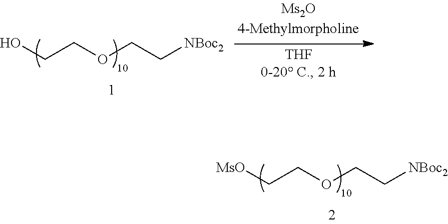

To a solution of tert-butyl N-tert-butoxy carbonyl-N-[2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl]carba mate (4.00 g, 5.70 mmol, 1.0 equiv) in THE (40 mL) were added 4-methylmorpholine (0.75 g, 7.41 mmol, 1.3 equiv) and methylsulfonyl methanesulfonate (1.49 g, 8.55 mmol, 1.5 equiv) at 0° C. The reaction mixture was diluted with water (50 mL) and the mixture was extracted with ethyl acetate (50 mL*3). The combined organic layer was washed with brine (50 mL), dried with anhydrous Na₂SO₄, filtered and concentrated in vacuo to afford 2-[2-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethoxy]ethoxy]ethyl methanesulfonate (4.45 g, crude) as a yellow oil.

### 2. Preparation of Compound 3

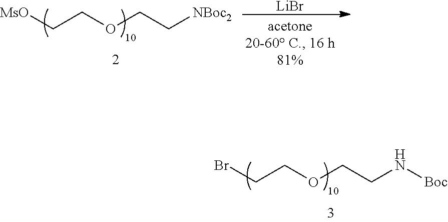

To a mixture of 2-[2-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethoxy]ethoxy]ethyl methanesulfonate (4.45 g, 5.71 mmol, 1.0 equiv) in acetone (50 mL) was added LiBr (4.96 g, 57.1 mmol, 10.0 equiv) at 20° C. Then the reaction mixture was stirred at 60° C. for 16 h. The reaction mixture was filtered and the filtrate was concentrated in vacuo. The residue was purified by column chromatography on silica gel (Dichloromethane/Methanol=100/1 to 25/1) to afford tert-butyl N-[2-[2-[2-[2-[2-[2-[2-[2-[2-[2-(2-bromoethoxy)ethoxy]ethoxy]ethoxy]ethoxy]ethoxy]ethoxy]ethoxy]ethoxy]ethoxy]ethyl]carbamate (3.07 g, 4.62 mmol, 81% yield) as a yellow oil. ¹H NMR (400 MHz, CDCl₃): δ 5.25-4.85 (m, 1H), 3.85-3.78 (m, 2H), 3.71-3.59 (m, 36H), 3.57-5.51 (m, 2H), 3.50-3.45 (m, 2H), 3.35-3.27 (m, 2H), 1.45 (s, 9H).

### 3. Preparation of Compound 5

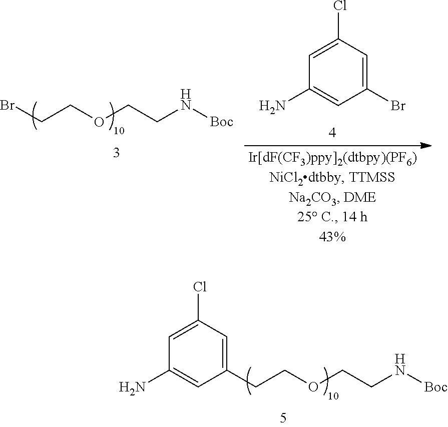

A mixture of 3-bromo-5-chloro-aniline (537 mg, 2.60 mmol, 1.0 equiv), tert-butyl N-[2-[2-[2-[2-[2-[2-[2-[2-[2-[2-(2-bromoethoxy)ethoxy]ethoxy]ethoxy]ethoxy]ethoxy]ethoxy]ethoxy]ethoxy]ethoxy]ethyl]carbamate (1.90 g, 2.86 mmol, 1.1 equiv), Ir[dF(CF₃)ppy]₂(dtbpy)(PF₆) (29.2 mg, 0.03 mmol, 0.01 equiv), NiCl₂.dtbbpy (5.17 mg, 0.01 mmol, 0.005 equiv), TTMSS (647 mg, 2.60 mmol, 1.0 equiv), and Na₂CO₃ (551 mg, 5.20 mmol, 2.0 equiv) in DME (26 mL) was stirred and irradiated with a 34 W blue LED lamp (7 cm away), with cooling fan to keep the reaction temperature at 25° C. for 14 h under N₂. The reaction mixture was filtered and the insoluble material was washed with ethyl acetate (20 mL). The combined filtrate was concentrated in vacuo. The residue was purified by column chromatography on silica gel (Petroleum ether/Ethyl acetate=1/1 to 0/1) to afford tert-butyl N-[2-[2-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethoxy]ethoxy]ethyl]carbamate (800 mg, 1.12 mmol, 43% yield) as a yellow oil. ¹H NMR (400 MHz, CDCl₃): δ 6.69-6.65 (m, 1H), 6.65-6.62 (m, 1H), 6.62-6.57 (m, 1H), 5.15-5.00 (m, 1H), 3.75-3.56 (m, 38H), 3.56-3.51 (m, 2H), 3.35-3.26 (m, 2H), 2.84-2.74 (m, 2H), 1.44 (s, 9H).

### 4. Preparation of Compound 7

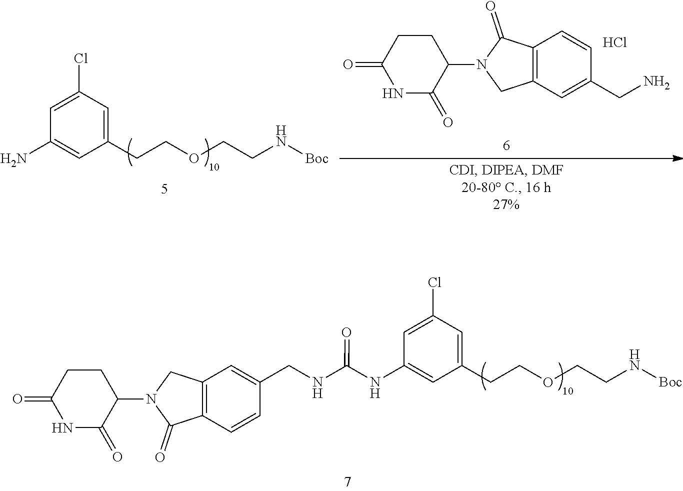

To a solution of 3-[5-(aminomethyl)-1-oxo-isoindolin-2-yl]piperidine-2,6-dione (348 mg, 1.12 mmol, 1.0 equiv, HCl salt) in DMF (10 mL) were added CDI (274 mg, 1.69 mmol, 1.5 equiv) and DIPEA (727 mg, 5.62 mmol, 5.0 equiv) at 20° C. The reaction mixture was stirred at 20° C. for 1 h. To the reaction mixture was added tert-butyl N-[2-[2-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethoxy]ethoxy]ethyl]carbamate (800 mg, 1.12 mmol, 1.0 equiv) and the reaction mixture was stirred at 80° C. for 15 h. The reaction mixture was diluted with water (50 mL) and extracted with ethyl acetate (30 mL*3). The combined organic phase was washed with brine (50 mL), dried with anhydrous Na₂SO₄, filtered and concentrated in vacuo. The residue was purified by column chromatography on silica gel (Dichloromethane/Methanol=100/1 to 25/1) to afford tert-butyl N-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethoxy]ethoxy]ethyl]carbamate (312 mg, 0.31 mmol, 27% yield) as a yellow oil. ¹H NMR (400 MHz, CDCl₃): δ 8.54 (s, 1H), 7.95 (s, 1H), 7.73-7.68 (m, 1H), 7.66-7.62 (m, 1H), 7.45 (s, 1H), 7.40-7.34 (m, 1H), 7.21-7.13 (m, 2H), 7.11-7.06 (m, 1H), 6.77-6.71 (m, 1H), 5.18-5.04 (m, 2H), 4.51-4.20 (m, 3H), 3.68-3.48 (m, 40H), 3.34-3.24 (m, 2H), 2.94-2.67 (m, 4H), 2.40-2.13 (m, 2H), 1.44 (s, 9H).

### 5. Preparation of Compound 8

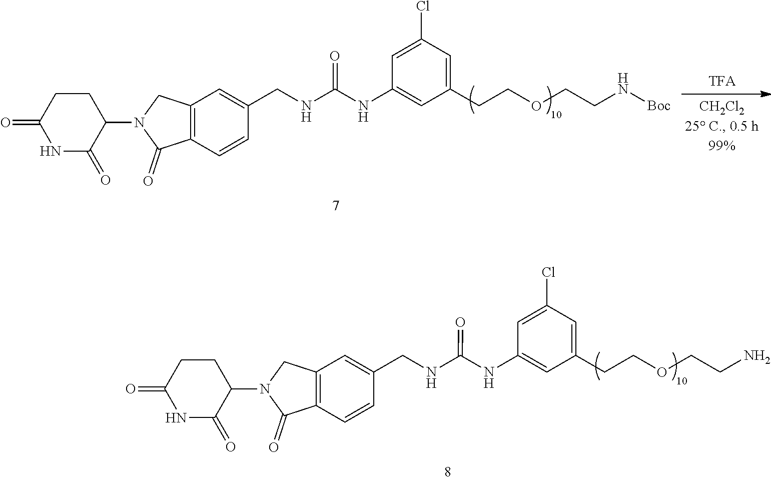

To a solution of tert-butyl N-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethoxy]ethoxy]ethyl]carbamate (0.45 g, 445 μmol, 1.0 equiv) in DCM (6 mL) was added TFA (4.62 g, 40.5 mmol, 3 mL, 91.0 equiv). The mixture was stirred at 25° C. for 0.5 h. The reaction mixture was concentrated in vacuo to afford 1-[3-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (0.45 g, 439 μmol, 99% yield, TFA salt) as a brown solid. LC-MS: MS (ES⁺): m/z=910.2 [M+H⁺].

### 6. Preparation of Compound 10

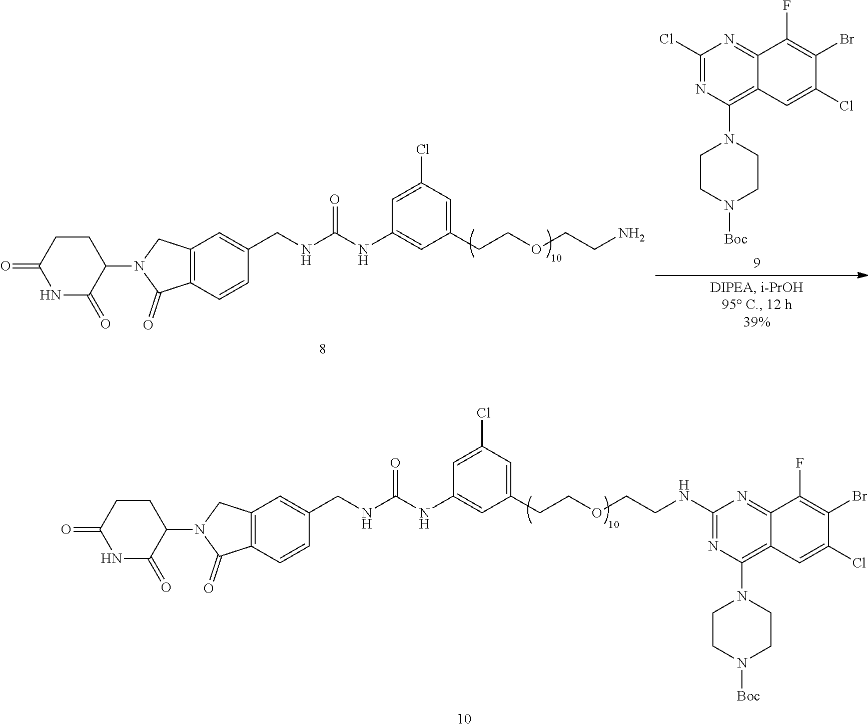

To a solution of 1-[3-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethyl]-5-chloro-phenyl]-3-[[2-(2,6-dioxo-3-piperidyl)-1-oxo-isoindolin-5-yl]methyl]urea (450 mg, 439 μmol, 1.0 equiv, TFA salt) in i-PrOH (12 mL) were added DIPEA (454 mg, 3.51 mmol, 0.60 mL, 8.0 equiv) and tert-butyl 4-(7-bromo-2,6-dichloro-8-fluoro-quinazolin-4-yl)piperazine-1-carboxylate (211 mg, 439 μmol, 1.0 equiv). The mixture was stirred at 95° C. for 12 h. The reaction mixture was concentrated in vacuo. The residue was purified by prep-HPLC (column: Waters Xbridge C18 150*50 mm*10 μm; mobile phase: [water (10 mM NH₄HCO₃)-ACN]; B %: 50%-80%) to afford tert-butyl 4-[7-bromo-6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (230 mg, 170 μmol, 39% yield) as a brown solid. ¹H NMR (400 MHz, CDCl₃): δ 7.81-7.73 (m, 1H), 7.71-7.56 (m, 2H), 7.48 (s, 1H), 7.43-7.39 (m, 1H), 7.09 (brs, 1H), 6.79 (s, 1H), 5.22-5.15 (m, 1H), 4.56-4.48 (m, 2H), 4.46-4.38 (m, 1H), 4.35-4.28 (m, 1H), 3.71-3.65 (m, 10H), 3.63-3.56 (m, 42H), 2.95-2.82 (m, 2H), 2.80-2.75 (m, 2H), 2.41-2.32 (m, 1H), 2.26-2.18 (m, 1H), 1.50 (s, 9H).

### 7. Preparation of Compound 12

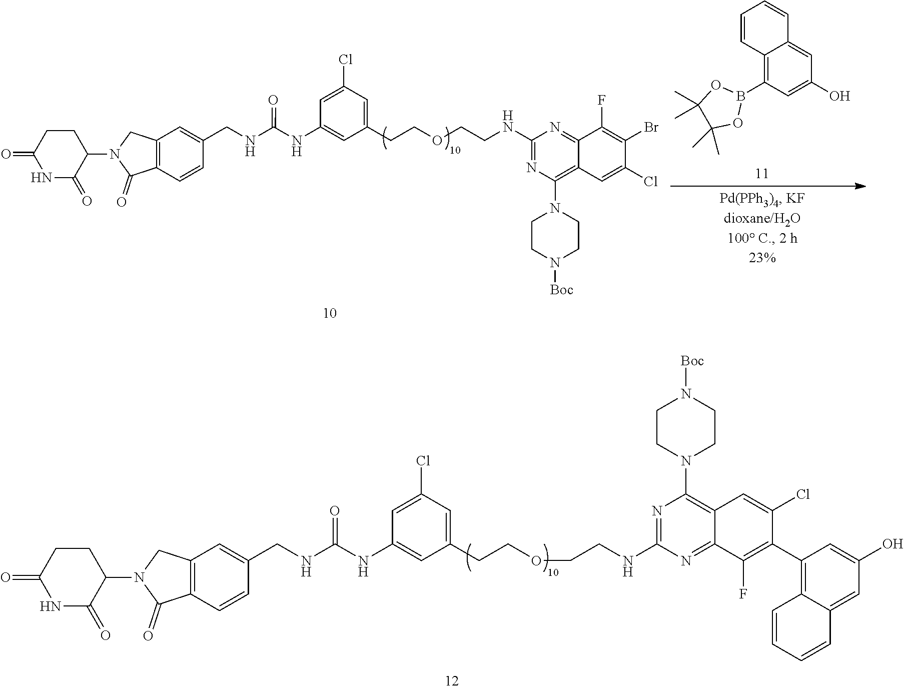

To a solution of tert-butyl 4-[7-bromo-6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethoxy]ethoxy]ethylamino]-8-fluoro-quinazolin-4-yl]piperazine-1-carboxylate (210 mg, 155 μmol, 1.0 equiv) in dioxane (20 mL) and H₂O (4 mL) were added Pd(PPh₃)₄ (89.6 mg, 77.5 μmol, 0.5 equiv), KF (22.5 mg, 388 μmol, 2.5 equiv) and 4-(4,4,5,5-tetramethyl-1,3,2-dioxaborolan-2-yl)naphthalen-2-ol (62.8 mg, 233 μmol, 1.5 equiv). The mixture was stirred at 100° C. for 2 h under N₂. The reaction mixture was diluted with water (30 mL) and extracted with CH₂Cl₂/MeOH (10/1, 20 mL*4). The combine organic phase was dried with anhydrous Na₂SO₄ and concentrated in vacuo. The residue was purified by prep-TLC on silica gel (CH₂Cl₂/MeOH=10/1) to afford tert-butyl4-[6-chloro-2-[2-[2-[2-[2-[2-[2-[2-[2-[2-[2-[2-[3-chloro-5-[[2-(2,6-dioxo-3-piperidyl)-1-oxo- isoindolin-5-yl]methyl carbamoylamino]phenyl]ethoxy]ethoxy]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (50.0 mg, 35.3 μmol, 23% yield) as a brown solid. ¹H NMR (400 MHz, DMSO-d₆): δ 10.97 (s, 1H), 9.97 (s, 1H), 8.80 (s, 1H), 7.83-7.74 (m, 2H), 7.72-7.65 (m, 1H), 7.54-7.49 (m, 2H), 7.46-7.39 (m, 2H), 7.28-7.19 (m, 3H), 7.09-7.01 (m, 2H), 6.88-6.79 (m, 2H), 5.15-5.06 (m, 1H), 4.47-4.39 (m, 3H), 4.34-4.27 (m, 1H), 3.61-3.54 (m, 11H), 3.53-3.45 (m, 42H), 2.76-2.70 (m, 2H), 2.64-2.59 (m, 2H), 1.44 (s, 9H). LC-MS: MS (ES⁺): RT=0.694 min, m/z=1418.3 [M+H⁺].

### 8. Preparation of Compound 13

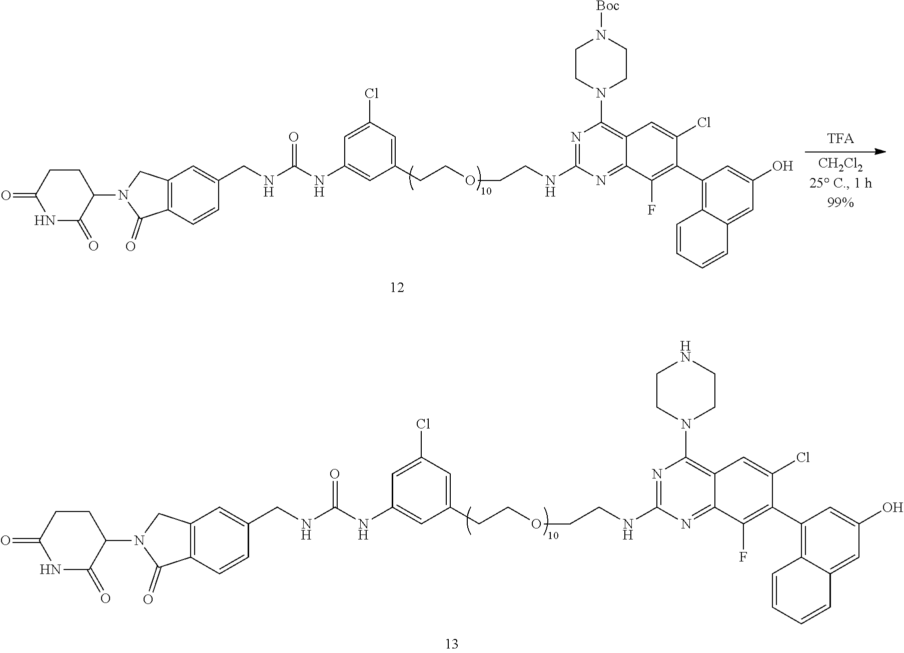

A mixture of tert-butyl 4-[6-chloro-2-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethoxy]ethoxy]ethylamino]-8-fluoro-7-(3-hydroxy-1-naphthyl)quinazolin-4-yl]piperazine-1-carboxylate (40.0 mg, 28.2 μmol, 1.0 equiv) in CH₂Cl₂ (0.4 mL) and TFA (0.2 mL) was stirred at 25° C. for 1 h. The reaction mixture was concentrated in vacuo to afford 1-[3-chloro-5-[2-[2-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (40.0 mg, 28.0 μmol, 99% yield, TFA salt) as a yellow solid. LC-MS: MS (ES⁺): RT=0.576 min, m/z=1318.3 [M+H⁺].

### 9. Preparation of I-16

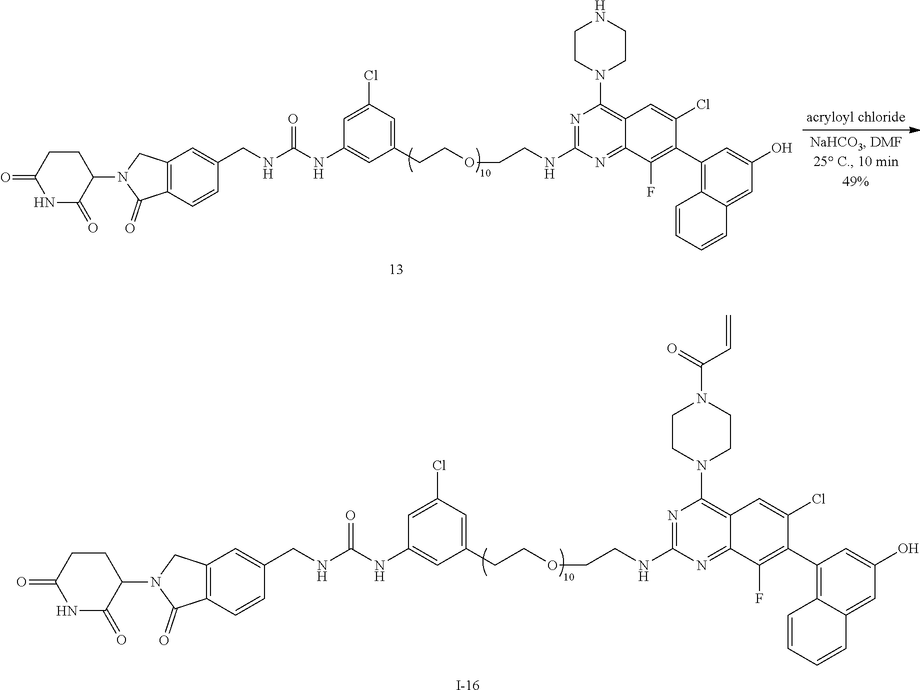

To a solution of 1-[3-chloro-5-[2-[2-[2-[2-[2-[2-[2-[2-[2-[2-[2-[[6-chloro-8-fluoro-7-(3-hydroxy-1-naphthyl)-4-piperazin-1-yl-quinazolin-2-yl]amino]ethoxy]ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (40.0 mg, 29.56 μmol, 1.0 equiv, TFA salt) and NaHCO₃ (24.8 mg, 296 μmol, 10 equiv) in THE (1 mL) and H₂O (0.2 mL) was added prop-2-enoyl chloride (2.67 mg, 29.6 μmol, 2.41 μL, 1.0 equiv) at 25° C. under N₂. The mixture was stirred at 25° C. for 10 min. The reaction mixture was diluted with water (20 mL) and extracted with EtOAc (20 ml*3). The combine organic phase was dried with anhydrous Na₂SO₄ and concentrated in vacuo. The residue was purified by prep-HPLC (column: Phenomenex Synergi C18 150*25 mm*10 μm; mobile phase: [water (0.225% FA)-ACN]; B %: 22%-52%, 10 min) to afford 1-[3-chloro-5-[2-[2-[2-[2-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]amino]ethoxy]ethoxy]ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (20.0 mg, 14.4 μmol, 49% yield, 99% purity) as an off-white solid. ¹H NMR (400 MHz, DMSO-d₆): δ 10.97 (brs, 1H), 10.37-9.82 (m, 1H), 9.11 (s, 1H), 7.84-7.73 (m, 2H), 7.71-7.65 (m, 1H), 7.55-7.49 (m, 2H), 7.46-7.40 (m, 2H), 7.29-7.20 (m, 3H), 7.19-7.13 (m, 1H), 7.11-7.02 (m, 2H), 6.90-6.79 (m, 2H), 6.21-6.14 (m, 1H), 5.80-5.69 (m, 1H), 5.15-5.06 (m, 1H), 4.47-4.27 (m, 4H), 3.89-3.67 (m, 7H), 3.59-3.53 (m, 6H), 3.53-3.48 (m, 12H), 3.48-3.45 (m, 22H), 3.37-3.28 (m, 4H), 2.97-2.84 (m, 1H), 2.75-2.70 (m, 2H), 2.63-2.59 (m, 1H), 2.43-2.31 (m, 1H), 2.03-1.93 (m, 1H). LC-MS: MS (ES⁺): RT=1.109 min, m/z=1372.6 [M+H⁺].

### Example 17—Preparation of Compound I-17

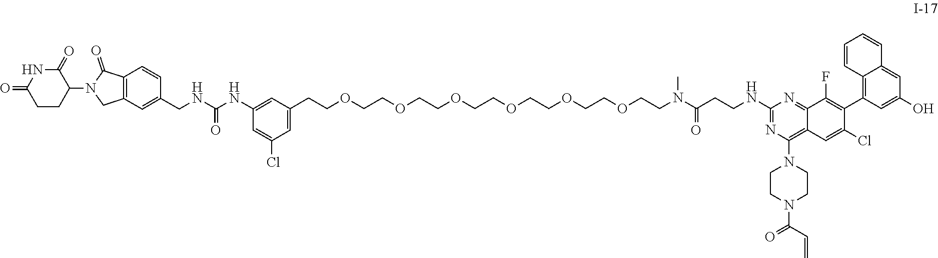 [SMILES: C=CC(=O)N1CCN(c2nc(NCCC(=O)N(C)CCOCCOCCOCCOCCOCCOCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

The title compound was prepared according to the following procedures.

### 1. Preparation of Compound 2

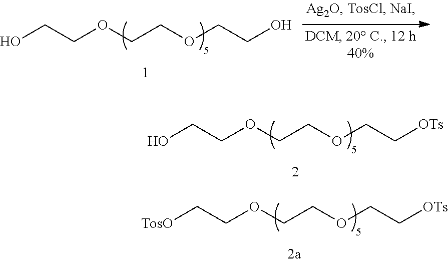

To a solution of compound 1 (10 g, 30.64 mmol, 1.0 equiv) in DCM (150 mL) was added Ag₂O (10.65 g, 45.96 mmol, 1.5 equiv), NaI (5 g, 33.36 mmol, 1.0 equiv) and TosCl (5.84 g, 30.64 mmol, 1.0 equiv). The mixture was stirred at 20° C. for 12 h. The reaction mixture was filtered and concentrated under reduced pressure to give a residue. The residue was purified by column chromatography (SiO₂, PE/EA=1/3 to 0/1 to EA/MeOH=20/1). Compound 2 (6.0 g, 12.49 mmol, 40% yield) was obtained as a yellow oil. Compound 2a (1.77 g, 2.79 mmol, 9% yield) was obtained as a yellow oil. ¹HNMR (400 MHz, CDCl₃): δ 7.81 (d, J=8.3 Hz, 2H), 7.35 (d, J=8.1 Hz, 2H), 4.19-4.14 (m, 2H), 3.76-3.57 (m, 26H), 2.46 (s, 3H).

### 2. Preparation of Compound 4

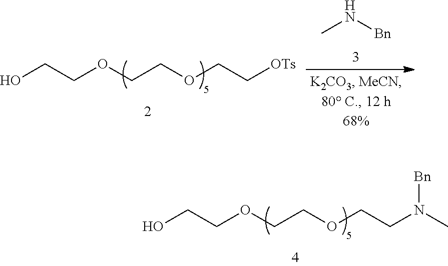

A mixture of compound 2 (6.0 g, 12.49 mmol, 1.0 equiv), compound 3 (3.03 g, 24.97 mmol, 3.22 mL, 2.0 equiv), K₂CO₃ (5.18 g, 37.46 mmol, 3.0 equiv) in MeCN (60 mL) was stirred at 80° C. for 12 h. The reaction mixture was filtered and concentrated under reduced pressure to give a residue. The residue was purified by prep-HPLC (column: Phenomenex luna C18 250*50 mm*10 μm; mobile phase: [water (10 mM NH₄HCO₃)-ACN]; B %: 30ACN %-60ACN %, 18 min). Compound 4 (3.7 g, 8.61 mmol, 68% yield) was obtained as a yellow oil. ¹HNMR (400 MHz, CDCl₃): δ 7.53-7.30 (m, 5H), 3.73-3.63 (m, 28H), 2.92-2.61 (m, 3H), 2.54-2.22 (m, 3H). LC-MS: MS (ES⁺): RT=0.813 min, m/z=430.3 [M+H⁺].

### 3. Preparation of Compound 5

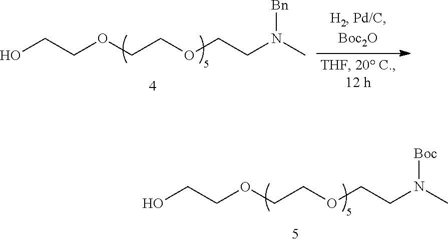

To a solution of compound 4 (3.7 g, 8.61 mmol, 1.0 equiv) and (Boc)₂O (2.07 g, 9.48 mmol, 2.1 mL, 1.1 equiv) in THF (60 mL) was added Pd/C (370 mg, 86 μmol, 10% purity, 0.10 equiv). The mixture was degassed and purged with H₂ for 3 times, and then the mixture was stirred at 20° C. for 12 h under H₂ atmosphere. The reaction mixture was filtered and concentrated under reduced pressure to give a residue. The product 5 (3.7 g, crude) was used into the next step without further purification. LC-MS: MS (ES⁺): RT=0.897 min, m/z=457.3 [M+18].

### 4. Preparation of Compound 6

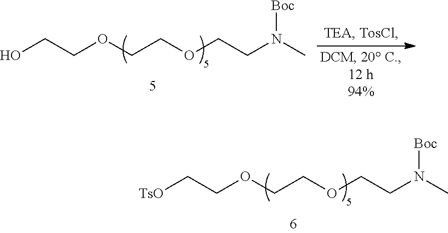

To a solution of compound 5 (3.7 g, 8.42 mmol, 1.0 equiv) in DCM (40 mL) was added Et₃N (2.54 g, 25.15 mmol, 3.5 mL, 2.9 equiv), TosCl (2.41 g, 12.63 mmol, 1.5 equiv). The mixture was stirred at 20° C. for 12 h. The reaction mixture was concentrated under reduced pressure to give a residue. The residue was purified by column chromatography (SiO₂, Ethyl acetate to DCM:MeOH=20/1). Compound 6 (4.7 g, 7.92 mmol, 94% yield) was obtained as a yellow oil. ¹HNMR (400 MHz, CDCl₃): δ 7.81 (d, J=8.3 Hz, 2H), 7.35 (d, J=8.1 Hz, 2H), 4.23-4.12 (m, 2H), 3.72-3.55 (m, 24H), 3.39 (m, 2H), 2.91 (s, 3H), 2.46 (s, 3H), 1.46 (s, 9H). LC-MS: MS (ES⁺): m/z=494.2 [M−Boc+H].

### 5. Preparation of Compound 7

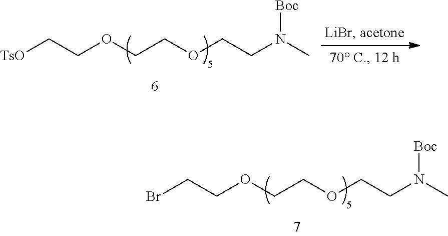

A mixture of compound 6 (4.37 g, 7.36 mmol, 1.0 equiv) and LiBr (3.20 g, 36.80 mmol, 923 μL, 5.0 equiv) in acetone (40 mL) was stirred at 70° C. for 12 h. The reaction mixture was partitioned between H₂O (20 mL) and EtOAc (50 mL) The organic phase was separated, washed with brine (20 mL), dried over Na₂SO₄, filtered and concentrated under reduced pressure to give a residue. The product 7 (4 g, crude) was used into the next step without further purification. ¹HNMR (400 MHz, CDCl₃): δ 3.82 (t, J=6.3 Hz, 2H), 3.70-3.58 (m, 22H), 3.48 (t, J=6.3 Hz, 2H), 3.43-3.35 (m, 2H), 2.91 (s, 3H), 1.46 (s, 9H).

### 6. Preparation of Compound 9

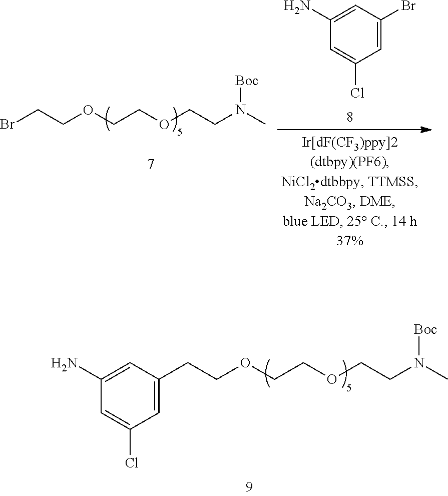

To an 40 mL vial equipped with a stir bar was added compound 8 (900 mg, 4.36 mmol, 1.0 equiv), compound 7 (2.85 g, 5.67 mmol, 1.3 equiv), Ir[dF(CF₃)ppy]₂(dtbpy)(PF₆) (48 mg, 43 μmol, 0.01 equiv), NiCl₂.dtbbpy (86 mg, 217 μmol, 0.05 equiv), TTMSS (1.08 g, 4.36 mmol, 1.3 mL, 1.0 equiv), Na₂CO₃ (924 mg, 8.72 mmol, 2.0 equiv) in DME (40 mL). The vial was sealed and placed under nitrogen was added. The reaction was stirred and irradiated with a 34 W blue LED lamp (7 cm away), with cooling fan to keep the reaction temperature at 25° C. for 14 h. The reaction mixture was filtered and concentrated under reduced pressure to give a residue. The residue was purified by column chromatography (SiO₂, PE/EtOAc=1/1 to EtOAc/MEOH=10/1). The residue was purified by prep-HPLC (column: Waters Xbridge C18 150*50 mm*10 μm; mobile phase: [water (10 mM NH₄HCO₃)-ACN]; B %: 39%-69%, min). Compound 9 (890 mg, 1.62 mmol, 37% yield) was obtained as a yellow oil. ¹HNMR (400 MHz, CDCl₃): δ 6.71-6.46 (m, 3H), 3.68-3.58 (m, 24H), 3.39 (m, 2H), 2.91 (s, 3H), 2.78 (t, J=6.9 Hz, 2H), 1.46 (s, 9H). LC-MS: MS (ES⁺): RT=0.900 min, m/z=549.3 [M+1].

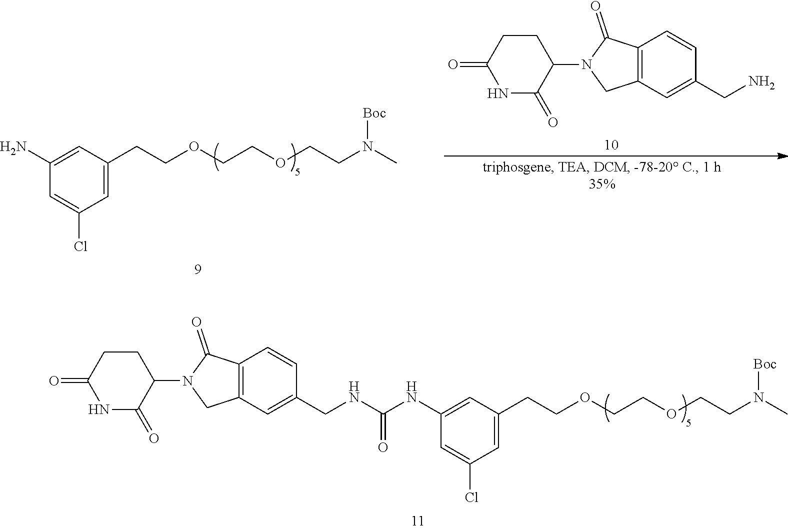

To a solution of triphosgene (70 mg, 235 μmol, 0.6 equiv) in DCM (20 mL) was added TEA (145 mg, 1.44 mmol, 0.2 mL, 3.9 equiv) and compound 9 (0.2 g, 364 μmol, 1.0 equiv) in DCM (20 mL) at −78° C., and then it was stirred for 0.5 h. Compound 10 (109 mg, 398 μmol, 1.1 equiv) was added at −78° C. The mixture was added and stirred at 20° C. for 0.5 h. The reaction mixture was poured into NaHCO₃ (50 mL) at 0° C., and then extracted with DCM (50 mL). The combined organic layers were washed with brine (50 mL), dried over Na₂SO₄, filtered and concentrated under reduced pressure to give a residue. The residue was purified by prep-TLC (SiO₂, DCM:MeOH=10:1). Compound 11 (110 mg, 129 μmol, 35% yield) was obtained as a yellow solid. LC-MS: MS (ES⁺): m/z=848.1 [M+1].

### 8. Preparation of Compound 12

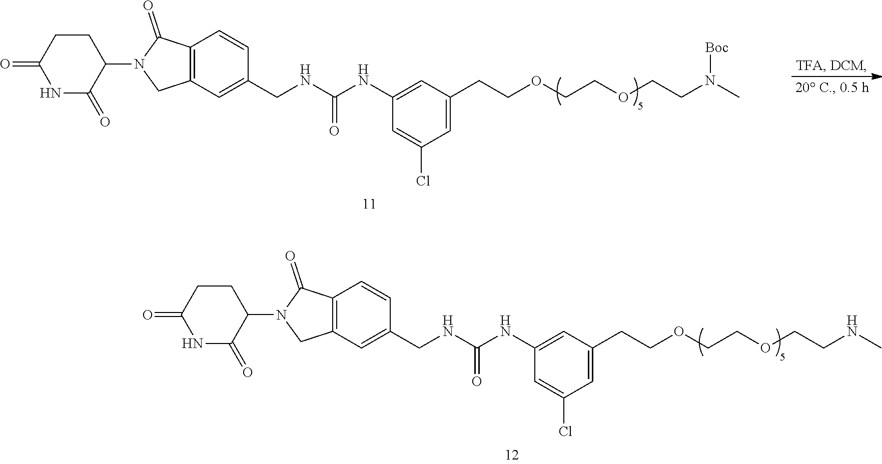

To a solution of compound 11 (50 mg, 58 μmol, 1.0 equiv) in DCM (2 mL) was added TFA (0.5 mL). The mixture was stirred at 20° C. for 0.5 h. The reaction mixture was concentrated under reduced pressure to give compound 12 (50 mg, crude, TFA salt) was obtained as a yellow oil. LC-MS: MS (ES⁺): RT=0.792 min, m/z=748.1 [M+1].

### 9. Preparation of I-17

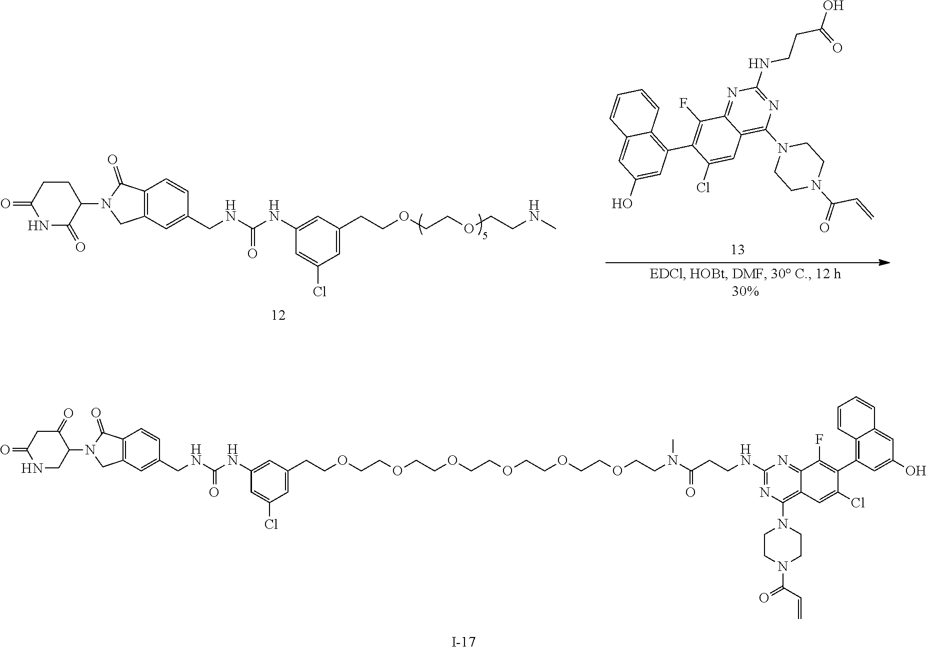

To a solution of compound 5 (40 mg, 67 μmol, 1.0 equiv, TFA salt) in DCM (1 mL) was added TEA (68 mg, 677 μmol, 94 μL, 10 equiv) and NaBH(OAc)₃ (143 mg, 677 μmol, 10 equiv). Then 4 (39 mg, 78 μmol, 1.1 equiv) was added. The mixture was stirred at 20° C. for 0.5 h. The reaction mixture was partitioned between brine (20 mL) and DCM (20 mL). The organic phase was separated, dried over Na₂SO₄, filtered and concentrated under reduced pressure to give a residue. The residue was purified by column chromatography by prep-TLC (SiO2, DCM:MeOH=9:1). Compound N-(4-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl) pyrrolidin-1-yl)butyl)-4-(((R)-8-cyclopentyl-7-ethyl-5-methyl-6-oxo-5,6,7,8-tetrahydropteridin-2-yl)amino)-3-methoxybenzamide (18 mg, 13 μmol, 30% yield, 94% purity) was obtained as a off-white solid. ¹HNMR (400 MHz, CD₃OD): δ 7.83 (s, 1H), 7.76-7.71 (m, 2H), 7.55-7.50 (s, 1H), 7.48-7.38 (m, 3H), 7.29-7.23 (m, 2H), 7.15 (s, 1H), 7.11-7.07 (s, 1H), 7.06-7.01 (s, 1H), 6.87-6.75 (m, 2H), 6.27 (m, 1H), 5.80 (m, 1H), 5.18-5.04 (m, 1H), 4.49 (s, 2H), 4.41 (m, 2H), 3.92-3.46 (m, 36H), 3.12-2.90 (m, 3H), 2.89-2.80 (m, 2H), 2.79-2.69 (m, 4H), 2.51-2.35 (m, 1H), 2.16-2.07 (m, 1H). LC-MS: MS (ES⁺): RT=2.046 min, m/z=1280.3, 1281.4 [M+H⁺]; LCMS method: LC-MS METHOD 25.M

### Example 18—Preparation of Compound I-18

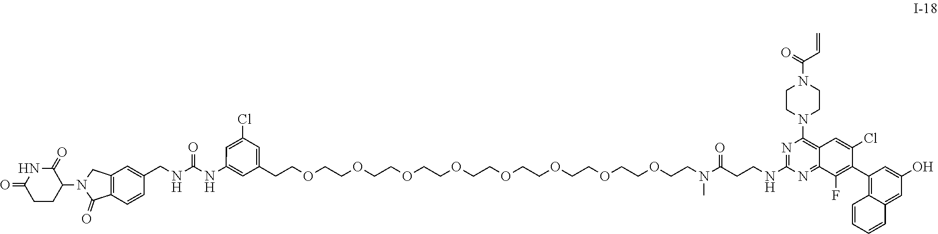 [SMILES: C=CC(=O)N1CCN(c2nc(NCCC(=O)N(C)CCOCCOCCOCCOCCOCCOCCOCCOCCc3cc(Cl)cc(NC(=O)NCc4ccc5c(c4)CN(C4CCC(=O)NC4=O)C5=O)c3)nc3c(F)c(-c4cc(O)cc5ccccc45)c(Cl)cc23)CC1]

The title compound was prepared according to the following procedures.

### 1. Preparation of Compound 2

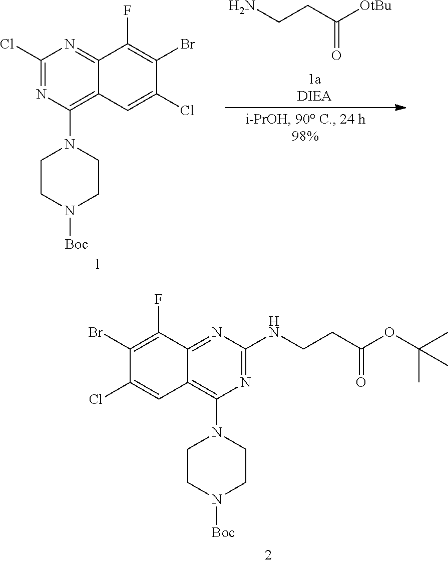 [SMILES: CC(C)(C)OC(=O)N1CCN(c2nc(Cl)nc3c(F)c(Br)c(Cl)cc23)CC1 ; CC(C)(C)OC(=O)CCN ; CC(C)(C)OC(=O)CCNc1nc(N2CCN(C(=O)OC(C)(C)C)CC2)c2cc(Cl)c(Br)c(F)c2n1]

To a solution of tert-butyl 4-(7-bromo-2,6-dichloro-8-fluoro-quinazolin-4-yl)piperazine-1-carboxylate (500 mg, 1.04 mmol, 1.0 equiv) and tert-butyl 3-aminopropanoate (151 mg, 1.04 mmol, 1.0 equiv) in i-PrOH (15 mL) was added DIEA (404 mg, 3.12 mmol, 544 μL, 3.0 equiv), and then it was stirred at 90° C. for 24 h. The reaction mixture was concentrated to afford crude product. The residue was purified by silica chromatography (Petroleum ether:Ethyl acetate=8:1-5:1) to afford tert-butyl 4-[7-bromo-2-[(3-tert-butoxy-3-oxo-propyl)amino]-6-chloro-8-fluoro-quinazolin-4-yl]piperazine-1-carboxylate (600 mg, 1.02 mmol, 98% yield) as a yellow solid. ¹H NMR: (400 MHz, CDCl₃) δ 7.57 (d, J=1.7 Hz, 1H), 5.72-5.54 (m, 1H), 3.80-3.72 (m, 2H), 3.70-3.54 (m, 8H), 2.58 (m, 2H), 1.50 (s, 9H), 1.47 (s, 9H). LC-MS: MS (ES⁺): RT=0.902 min, m/z=590.0 [M+H⁺].

### 2. Preparation of Compound 4

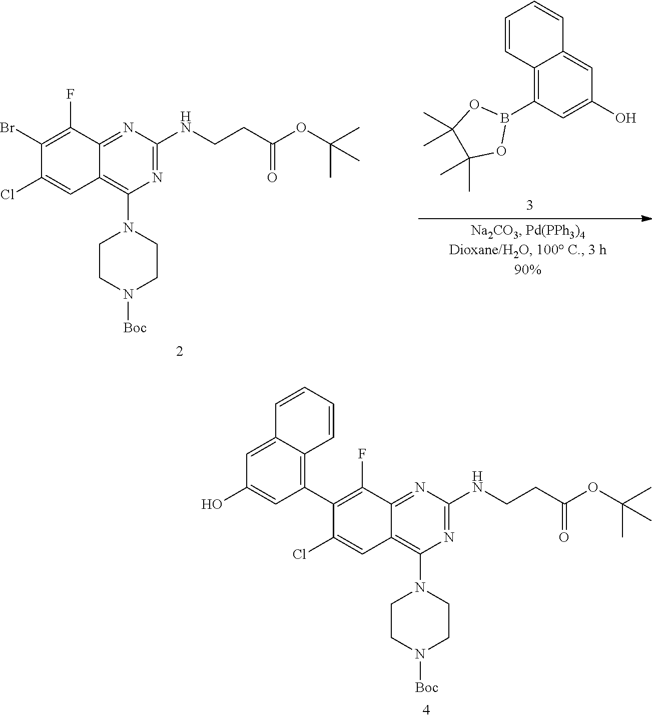 [SMILES: CC(C)(C)OC(=O)CCNc1nc(N2CCN(C(=O)OC(C)(C)C)CC2)c2cc(Cl)c(Br)c(F)c2n1 ; CC1(C)OB(c2cc(O)cc3ccccc23)OC1(C)C ; CC(C)(C)OC(=O)CCNc1nc(N2CCN(C(=O)OC(C)(C)C)CC2)c2cc(Cl)c(-c3cc(O)cc4ccccc34)c(F)c2n1]

To a solution of tert-butyl 4-[7-bromo-2-[(3-tert-butoxy-3-oxo-propyl)amino]-6-chloro-8-fluoro-quinazolin-4-yl]piperazine-1-carboxylate (0.66 g, 1.12 mmol, 1.0 equiv) and 4-(4,4,5,5-tetramethyl-1,3,2-dioxaborolan-2-yl)naphthalen-2-ol (454 mg, 1.68 mmol, 1.5 equiv) in dioxane (8 mL) and H₂O (2 mL) was added Na₂CO₃ (297 mg, 2.80 mmol, 2.5 equiv) and Pd(PPh₃)₄ (259 mg, 224 μmol, 0.2 equiv), and then it was stirred at 100° C. for 3 h. The reaction mixture was concentrated to afford crude product. The residue was purified by silica chromatography (Petroleum ether:Ethyl acetate=5:1-3:1) to afford tert-butyl 4-[2-[(3-tert-butoxy-3-oxo-propyl)amino]-6-chloro-8-fluoro-7-(3-hydroxy-1-naphthyl)quinazolin-4-yl]piperazine-1-carboxylate (0.66 g, 1.01 mmol, 90% yield) as a yellow solid. ¹H NMR: (400 MHz, CDCl₃) δ 7.73 (d, J=8.2 Hz, 1H), 7.64 (s, 1H), 7.40 (t, J=7.5 Hz, 1H), 7.32-7.28 (m, 2H), 7.23-7.17 (m, 1H), 7.13 (s, 1H), 3.88-3.56 (m, 10H), 2.59 (t, J=6.4 Hz, 2H), 1.51 (s, 9H), 1.44 (s, 9H). LC-MS: MS (ES⁺): RT=0.683 min, m/z=652.3 [M+H⁺].

### 3. Preparation of Compound 5

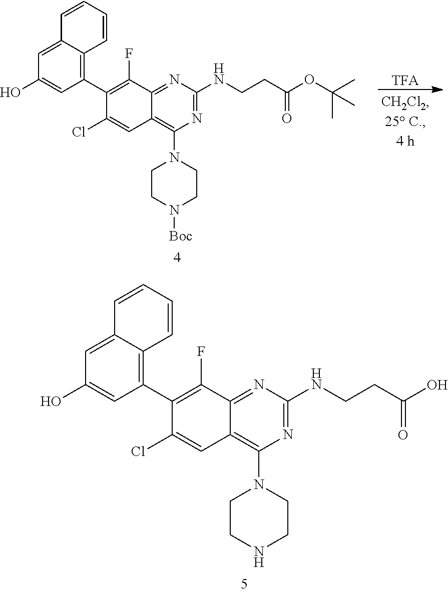 [SMILES: CC(C)(C)OC(=O)CCNc1nc(N2CCN(C(=O)OC(C)(C)C)CC2)c2cc(Cl)c(-c3cc(O)cc4ccccc34)c(F)c2n1 ; O=C(O)CCNc1nc(N2CCNCC2)c2cc(Cl)c(-c3cc(O)cc4ccccc34)c(F)c2n1]

To a solution of tert-butyl 4-[2-[(3-tert-butoxy-3-oxo-propyl)amino]-6-chloro-8-fluoro-7-(3-hydroxy-1-naphthyl)quinazolin-4-yl]piperazine-1-carboxylate (660 mg, 1.01 mmol, 1.0 equiv) in CH₂Cl₂ (2 mL) was added TFA (1 mL), and then it was stirred at 25° C. for 4 h. The reaction mixture was concentrated to afford crude product. 3-[[6-chloro-8-fluoro-7-(3-hydroxy-1-naphthyl)-4-piperazin-1-yl-quinazolin-2-yl]amino]propanoic acid (617 mg, 1.01 mmol, 100% yield, TFA salt) was obtained as a yellow oil and used for the next step directly. LC-MS: MS (ES⁺): m/z=496.1 [M+H⁺].

### 4. Preparation of Compound 6

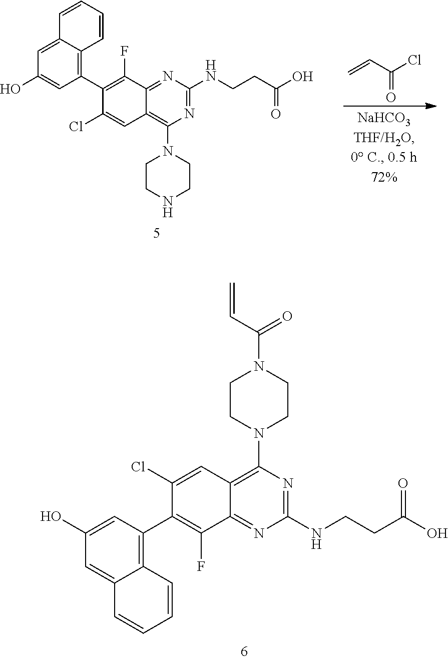

To a solution of 3-[[6-chloro-8-fluoro-7-(3-hydroxy-1-naphthyl)-4-piperazin-1-yl-quinazolin-2-yl]amino]propanoic acid (617 mg, 1.01 mmol, 1.0 eq, TFA salt) in THE (10 mL) was added NaHCO₃ (850 mg, 10.1 mmol, 10.0 equiv) in H₂O (10 mL), then prop-2-enoyl chloride (92 mg, 1.0 mmol, 83 μL, 1.0 equiv) was added to the solution and stirred at 0° C. for 0.5 h. The reaction mixture was added 20 mL water, and then it was extracted with EtOAc/THF (5:1, 100 mL×5). The organic layers were dried over anhydrous Na₂SO₄, filtered and concentrated to afford crude product. The residue was triturated by THE (5 mL) to afford 3-[[6-chloro-8-fluoro-7-(3-hydroxy-1-naphthyl)-4-(4-prop-2-enoylpiperazin-1-yl)quinazolin-2-yl]amino]propanoic acid (400 mg, 727 μmol, 72% yield) as a yellow solid. ¹H NMR: (400 MHz, DMSO-d6) δ 7.85-7.73 (m, 2H), 7.62-7.38 (m, 2H), 7.36-7.14 (m, 4H), 7.05 (d, J=2.2 Hz, 1H), 6.84 (dd, J=16.7 Hz, 1H), 6.17 (m, 1H), 5.89-5.64 (m, 1H), 3.90-3.64 (m, 10H), 2.41 (t, J=6.8 Hz, 2H). LC-MS: MS (ES⁺): RT=0.704 min, m/z=550.2 [M+H⁺].

### 5. Preparation of Compound 8

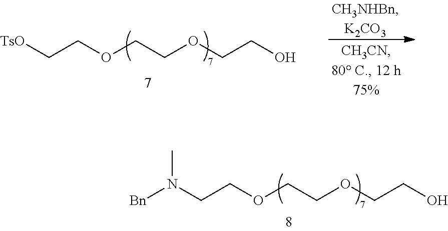

To a solution of 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (4.40 g, 7.74 mmol, 1.0 equiv) in CH₃CN (40 mL) was added K₂CO₃ (3.21 g, 23.2 mmol, 3.0 equiv) and N-methyl-1-phenyl-methanamine (1.88 g, 15.5 mmol, 2.00 mL, 2.0 equiv), and then it was stirred at 80° C. for 12 h. The reaction mixture was filtered, and the filtrate was concentrated to afford crude product. The residue was purified by prep-HPLC (column: Phenomenex luna C18 250*50 mm*10 μm; mobile phase: [water (10 mM NH₄HCO₃)-ACN]; B %: 30ACN %-60ACN %, 18 min) to afford 2-[2-[2-[2-[2-[2-[2-[2-[2-[benzyl(methyl)amino]ethoxy]ethoxy]ethoxy]ethoxy]ethoxy]ethoxy]ethoxy]ethoxy]ethanol (3.0 g, 5.8 mmol, 75% yield) as a yellow oil. ¹H NMR: (400 MHz, CDCl₃) δ 7.33-7.22 (m, 5H), 3.72 (m, 2H), 3.69-3.60 (m, 32H), 3.55 (s, 2H), 2.82 (br s, 1H), 2.62 (t, J=6.1 Hz, 2H), 2.26 (s, 3H). LC-MS: MS (ES⁺): RT=0.868 min, m/z=518.3 [M+H⁺].

### 6. Preparation of Compound 9

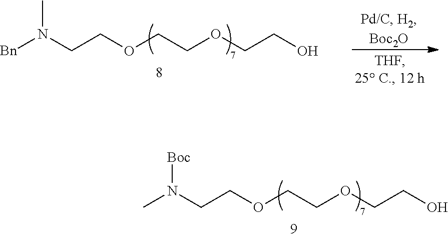

To a solution of 2-[2-[2-[2-[2-[2-[2-[2-[2-[benzyl(methyl)amino]ethoxy]ethoxy]ethoxy]ethoxy]ethoxy]ethoxy]ethoxy]ethoxy]ethanol (3.0 g, 5.8 mmol, 1.0 equiv) in THF (20 mL) was added Pd/C (600 mg, 10% purity) and Boc₂O (1.39 g, 6.37 mmol, 1.46 mL, 1.1 equiv), and then it was degassed and purged with H₂. The reaction mixture was stirred at 25° C. for 12 h under 15 psi pressure. The reaction mixture was filtered, and the filtrate was concentrated to afford tert-butyl N-[2-[2-[2-[2-[2-[2-[2-[2-(2-hydroxyethoxy)ethoxy]ethoxy]ethoxy]ethoxy]ethoxy]ethoxy]ethoxy]ethyl]-N-methyl-carbamate (3.1 g, crude) as a colorless oil and used for the next step directly. ¹H NMR: (400 MHz, CDCl₃) δ 3.75-3.72 (m, 2H), 3.70-3.58 (m, 32H), 3.42-3.36 (m, 2H), 2.92 (s, 3H), 1.46 (s, 9H).

### 7. Preparation of Compound 10

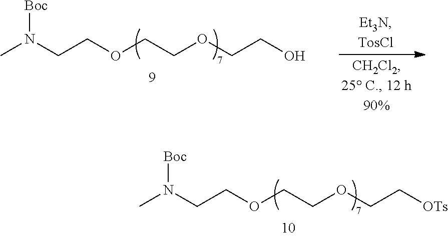

To a solution of tert-butyl N-[2-[2-[2-[2-[2-[2-[2-[2-(2-hydroxyethoxy)ethoxy]ethoxy]ethoxy]ethoxy]ethoxy]ethoxy]ethoxy]ethyl]-N-methyl-carbamate (3.10 g, 5.88 mmol, 1.0 equiv) and Et₃N (1.19 g, 11.8 mmol, 1.60 mL, 2.0 equiv) in CH₂Cl₂ (20 mL) was added TosCl (1.68 g, 8.81 mmol, 1.5 equiv), and then it was stirred at 25° C. for 12 h. The reaction mixture was concentrated to afford crude product. The residue was purified by silica chromatography (Petroleum ether:Ethyl acetate=1:1-0:1) to afford 2-[2-[2-[2-[2-[2-[2-[2-[2-[tert-butoxycarbonyl(methyl)amino]ethoxy]ethoxy]ethoxy]ethoxy]ethoxy]ethoxy]ethoxy]ethoxy]ethyl 4-methylbenzenesulfonate (3.60 g, 5.28 mmol, 90% yield) as a colorless oil. ¹H NMR: (400 MHz, CDCl₃) δ 7.80 (d, J=8.4 Hz, 2H), 7.35 (d, J=8.1 Hz, 2H), 4.21-4.13 (m, 2H), 3.70 (m, 2H), 3.67-3.55 (m, 30H), 3.39 (m, 2H), 2.91 (s, 3H), 2.45 (s, 3H), 1.46 (s, 9H).

### 8. Preparation of Compound 11

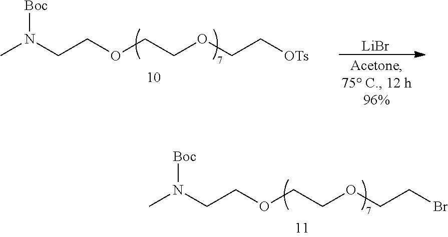

To a solution of 2-[2-[2-[2-[2-[2-[2-[2-[2-[tert-butoxycarbonyl(methyl)amino]ethoxy]ethoxy]ethoxy]ethoxy]ethoxy]ethoxy]ethoxy]ethoxy]ethyl 4-methylbenzenesulfonate (3.60 g, 5.28 mmol, 1.0 equiv) in acetone (30 mL) was added LiBr (2.29 g, 26.4 mmol, 5.0 equiv), and then it was stirred at 75° C. for 12 hr. The reaction mixture was poured into 100 mL water, and then it was extracted with EtOAc (3×30 mL). The organic layers were dried over anhydrous Na₂SO₄, filtered and concentrated to afford tert-butyl N-[2-[2-[2-[2-[2-[2-[2-[2-(2-bromoethoxy)ethoxy]ethoxy]ethoxy]ethoxy]ethoxy]ethoxy]ethoxy]ethyl]-N-methyl-carbamate (3.0 g, 5.1 mmol, 96% yield) as a yellow oil. ¹H NMR: (400 MHz, CDCl₃) δ 3.81 (t, J=6.3 Hz, 2H), 3.70-3.55 (m, 30H), 3.48 (t, J=6.3 Hz, 2H), 3.44-3.35 (m, 2H), 2.91 (s, 3H), 1.45 (s, 9H).

### 9. Preparation of Compound 13

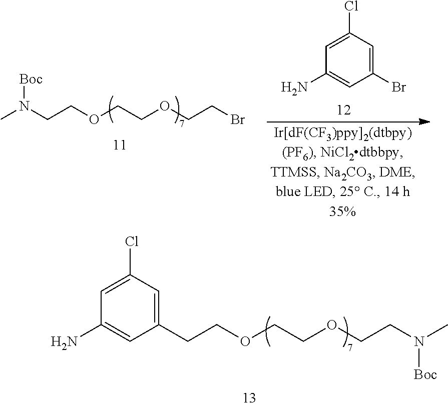

To a solution of 3-bromo-5-chloro-aniline (540 mg, 2.62 mmol, 1.0 equiv) and tert-butyl N-[2-[2-[2-[2-[2-[2-[2-[2-(2-bromoethoxy)ethoxy]ethoxy]ethoxy]ethoxy]ethoxy]ethoxy]ethoxy]ethyl]-N-methyl-carbamate (1.70 g, 2.88 mmol, 1.1 equiv) in DME (24 mL) was added Ir[dF(CF₃)ppy]₂(dtbpy)(PF₆) (29 mg, 26 μmol, 0.01 equiv), NiCl₂.dtbbpy (5.2 mg, 13 μmol, 0.005 equiv), Na₂CO₃ (555 mg, 5.23 mmol, 2.0 equiv) and TTMSS (651 mg, 2.62 mmol, 807 μL, 1.0 equiv), and then it was stirred and irradiated with a 34 W blue LED lamp (7 cm away), with cooling fan to keep the reaction temperature at 25° C. for 14 h. The reaction mixture was filtered and the filtrate was concentrated to afford crude product. The residue was purified by prep-HPLC (column: Waters Xbridge C18 150*50 mm*10 μm; mobile phase: [water (10 mM NH₄HCO₃)-ACN]; B %: 39%-69%) to afford tert-butyl N-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethyl]-N-methyl-carbamate (590 mg, 926 μmol, 35% yield) as a light yellow oil. ¹H NMR (400 MHz, CDCl₃) δ 7.09-6.64 (m, 3H), 3.74-3.52 (m, 32H), 3.38 (m, 2H), 2.91 (s, 3H), 2.80 (t, J=6.6 Hz, 2H), 1.45 (s, 9H). LC-MS: MS (ES⁺): RT=0.891 min, m/z=637.5 [M+H⁺].

### 10. Preparation of Compound 15

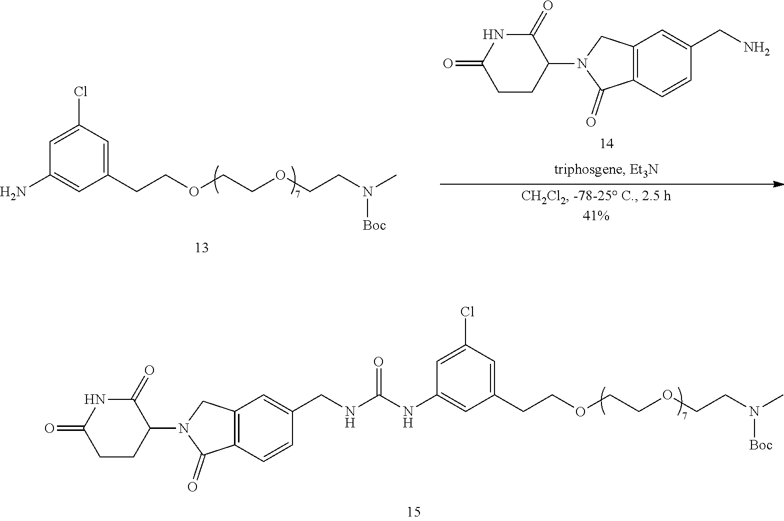

To a solution of triphosgene (70 mg, 0.24 mmol, 0.5 equiv) and Et₃N (143 mg, 1.41 mmol, 197 μL, 3.0 equiv) in CH₂Cl₂ (3 mL) was slowly added tert-butyl N-[2-[2-[2-[2-[2-[2-[2-[2-[2-(3-amino-5-chloro-phenyl)ethoxy]ethoxy]ethoxy]ethoxy]ethoxy]ethoxy]ethoxy]ethoxy]ethyl]-N-methyl-carbamate (300 mg, 471 μmol, 1.0 equiv) in CH₂Cl₂ (2 mL) at −78° C., and then it was stirred for 0.5 h. 3-[5-(aminomethyl)-1-oxo-isoindolin-2-yl]piperidine-2,6-dione (175 mg, 565 μmol, 1.2 equiv, HCl salt) was added to the mixture, then it was slowly warmed to 25° C. and stirred for 2 hr. The reaction mixture was poured into 30 mL sat. NaHCO₃, and then it was extracted with CH₂Cl₂ (2×30 mL). The organic layers were dried over anhydrous Na₂SO₄, filtered and concentrated to afford crude product. The residue was purified by silica chromatography (CH₂Cl₂:MeOH=50:1-30:1) to afford tert-butyl N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethyl]-N-methyl-carbamate (180 mg, 192 μmol, 41% yield) as a yellow oil. LC-MS: MS (ES⁺): RT=0.705 min, m/z=936.2 [M+H⁺].

### 11. Preparation of Compound 16

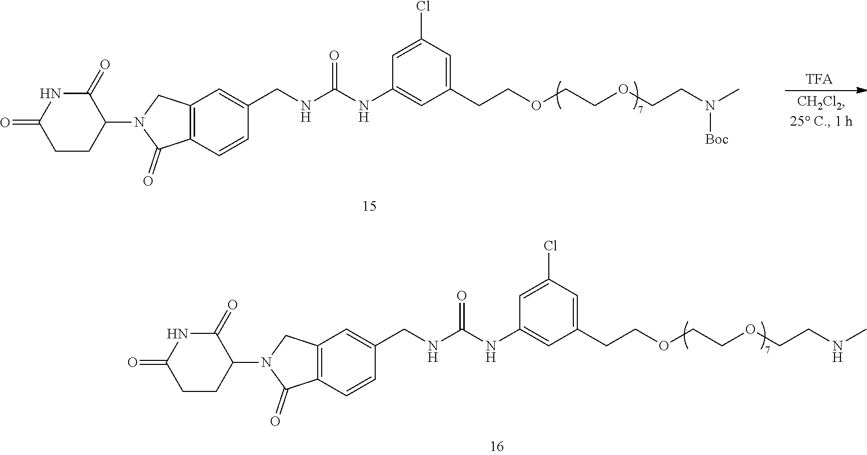

To a solution of tert-butyl N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethyl]-N-methyl-carbamate (70 mg, 75 μmol, 1.0 equiv) in CH₂Cl₂ (1 mL) was added TFA (0.5 mL), and then it was stirred at 25° C. for 1 h. The reaction mixture was concentrated to afford 1-[3-chloro-5-[2-[2-[2-[2-[2-[2-[2-[2-[2-(methylamino) ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (70 mg, 74 μmol, 99% yield, TFA salt) as a yellow oil and used for the next step directly. LC-MS: MS (ES⁺): RT=0.691 min, m/z=836.6 [M+H⁺].

### 12. Preparation of I-18

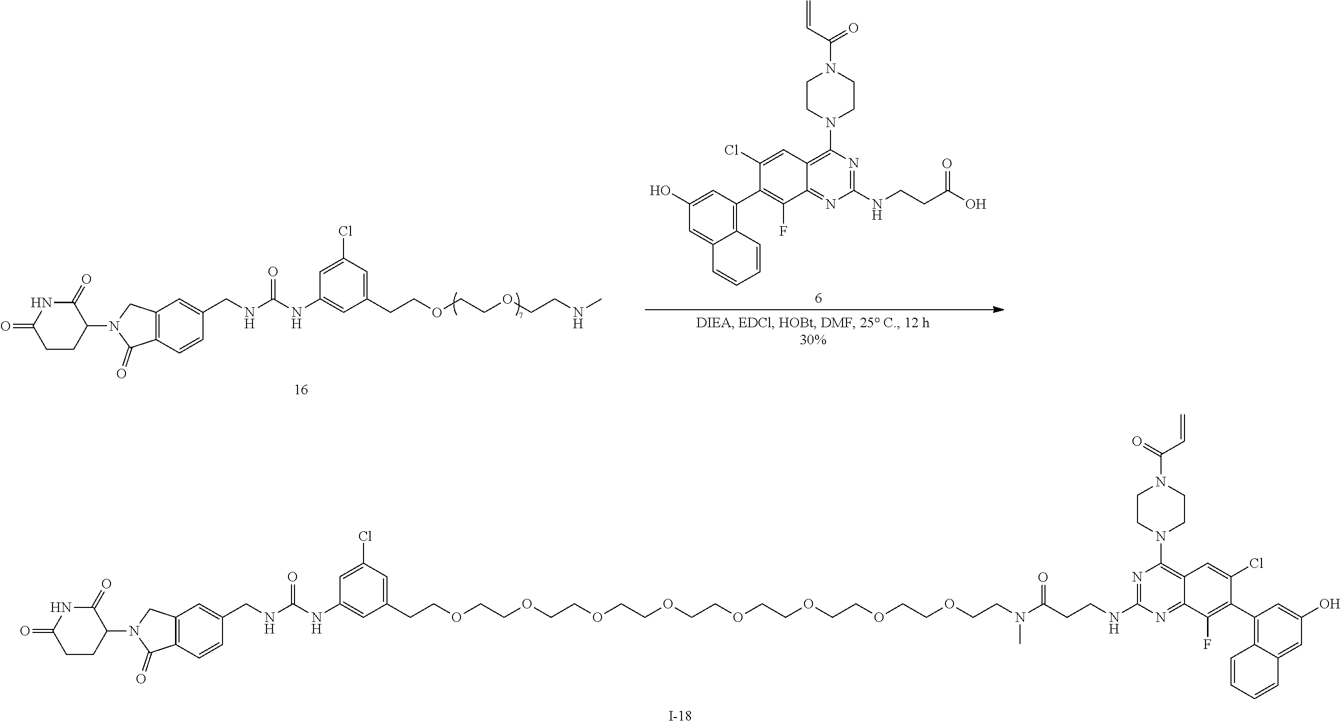

To a solution of 1-[3-chloro-5-[2-[2-[2-[2-[2-[2-[2-[2-[2-(methylamino)ethoxy]ethoxy]ethoxy]ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (70 mg, 74 μmol, 1.0 equiv, TFA salt) and 3-[[6-chloro-8-fluoro-7-(3-hydroxy-1-naphthyl)-4-(4-prop-2-enoylpiperazin-1-yl)quinazolin-2-yl]amino]propanoic acid (41 mg, 74 μmol, 1.0 equiv) in DMF (1.5 mL) was added DIEA (29 mg, 0.22 mmol, 39 μL, 3.0 equiv), EDCI (17 mg, 88 μmol, 1.2 equiv) and HOBt (12 mg, 88 μmol, 1.2 equiv), and then it was stirred at 25° C. for 12 h. The reaction mixture was quenched by 0.1 mL water. The residue was purified by prep-HPLC (column: Phenomenex Gemini-NX C18 75*30 mm*3 μm; mobile phase: [water (0.225% FA)-ACN]; B %: 25%-55%, 7 min) to afford N-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethoxy]ethoxy]ethyl]-3-[[6-chloro-8-fluoro-7-(3-hydroxy-1-naphthyl)-4-(4-prop-2-enoylpiperazin-1-yl)quinazolin-2-yl]amino]-N-methyl-propanamide (30 mg, 22 μmol, 30% yield) as a white solid. ¹H NMR: (400 MHz, CD₃OD) δ 7.90-7.80 (brs, 1H), 7.74 (d, J=8.0 Hz, 2H), 7.51 (s, 1H), 7.48-7.37 (m, 3H), 7.29-7.23 (m, 2H), 7.22-7.15 (m, 1H), 7.09 (s, 1H), 7.04 (s, 1H), 6.87-6.73 (m, 2H), 6.27 (d, J=16.9 Hz, 1H), 5.84-5.75 (m, 1H), 5.11 (m, 1H), 4.49 (s, 2H), 4.47-4.35 (m, 2H), 3.93-3.44 (m, 44H), 3.14-2.91 (m, 3H), 2.89-2.80 (m, 2H), 2.80-2.70 (m, 4H), 2.48-2.35 (m, 1H), 2.17-2.08 (m, 1H). LC-MS: MS (ES⁺): RT=2.190 min, m/z=684.5 [M/2+H⁺], LCMS method: LC-MS METHOD 25.

### Example 19—Preparation of Compound I-19

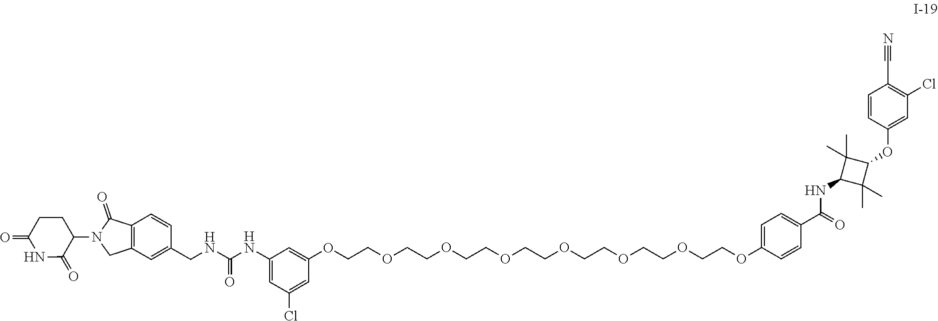 [SMILES: CC1(C)[C@H](NC(=O)c2ccc(OCCOCCOCCOCCOCCOCCOCCOc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1]

The title compound was prepared according to the following procedures.

### 1. Preparation of Compound 3

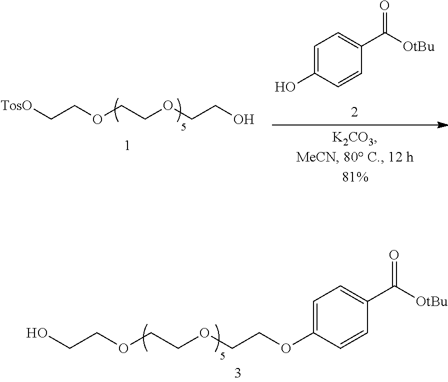

To a solution of compound 1 (6.6 g, 13.7 mmol, 1.0 equiv) in MeCN (60 mL) was added K₂CO₃ (5.7 g, 41.2 mmol, 3.0 equiv) and compound 2 (2.7 g, 13.7 mmol, 1.0 equiv). The mixture was stirred at 80° C. for 12 h. The reaction mixture was filtered and concentrated under reduced pressure to give a residue. The residue was purified by column chromatography (SiO₂, Ethyl acetate/MeOH=1/0 to 10/1). Compound 3 (5.6 g, 11.1 mmol, 81% yield) was obtained as a yellow oil. ¹HNMR (400 MHz, CDCl₃): δ7.99-7.88 (m, 2H), 6.96-6.86 (m, 2H), 4.20-4.15 (m, 2H), 3.91-3.86 (m, 2H), 3.76-3.71 (m, 4H), 3.71-3.64 (m, 18H), 3.63-3.59 (m, 2H), 1.59 (s, 9H). LC-MS: MS (ES⁺): RT=0.883 min, m/z=503.2 [M+H⁺].

### 2. Preparation of Compound 4

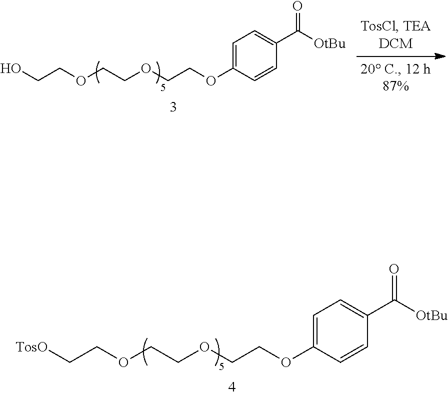

To a solution of compound 3 (5.6 g, 11.14 mmol, 1.0 equiv) in DCM (60 mL) was added Et₃N (3.3 g, 33.05 mmol, 4.6 mL, 2.9 equiv) and TosCl (3.2 g, 16.78 mmol, 1.5 equiv). The mixture was stirred at 20° C. for 12 h. The reaction mixture was concentrated under reduced pressure to give a residue. The residue was purified by column chromatography (SiO₂, Petroleum ether/Ethyl acetate=1/10 to 0/1). Compound 4 (6.4 g, 9.74 mmol, 87% yield) was obtained as a yellow oil. ¹HNMR (400 MHz, CDCl₃): δ 7.96-7.90 (m, 2H), 7.81 (d, J=8.4 Hz, 2H), 7.35 (d, J=8.0 Hz, 2H), 6.95-6.85 (m, 2H), 4.21-4.13 (m, 4H), 3.91-3.85 (m, 2H), 3.75-3.71 (m, 2H), 3.71-3.62 (m, 16H), 3.60-3.57 (m, 4H), 2.45 (s, 3H), 1.59 (s, 9H).

### 3. Preparation of Compound 6

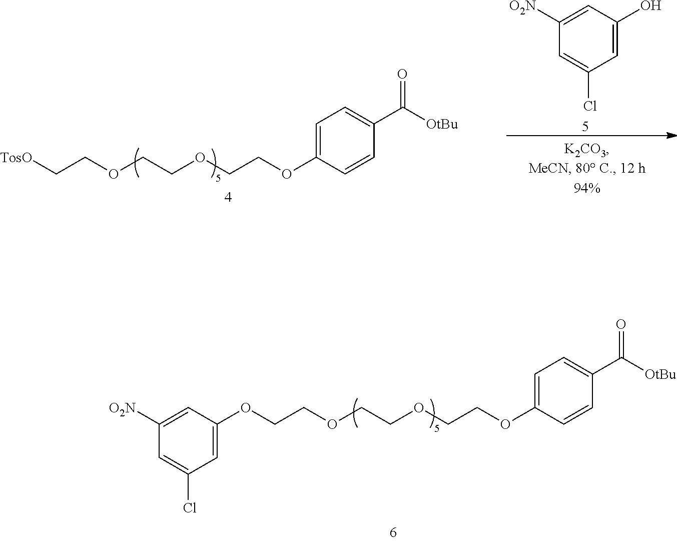

To a solution of compound 4 (529 mg, 3.05 mmol, 1.0 equiv) and compound 5 (529 mg, 3.05 mmol, 1.0 equiv) in MeCN (20 mL) was added K₂CO₃ (1.26 g, 9.14 mmol, 3.0 equiv). The mixture was stirred at 80° C. for 12 h. The reaction mixture was filtered and concentrated under reduced pressure to give a residue. The residue was purified by column chromatography (SiO₂, Petroleum ether/Ethyl acetate=1/1 to 0/1). Compound 6 (1.89 g, 2.87 mmol, 94% yield) was obtained as a yellow oil. ¹HNMR (400 MHz, CDCl₃): δ 7.93 (d, J=8.8 Hz, 2H), 7.82 (t, J=1.6 Hz, 1H), 7.68 (t, J=2.0 Hz, 1H), 7.26-7.24 (m, 1H), 6.91 (d, J=8.9 Hz, 2H), 4.28-4.15 (m, 4H), 3.96-3.84 (m, 4H), 3.78-3.61 (m, 20H), 1.61-1.58 (s, 9H). LC-MS: MS (ES⁺): RT=1.058 min, m/z=675.1 [M+18].

### 4. Preparation of Compound 7

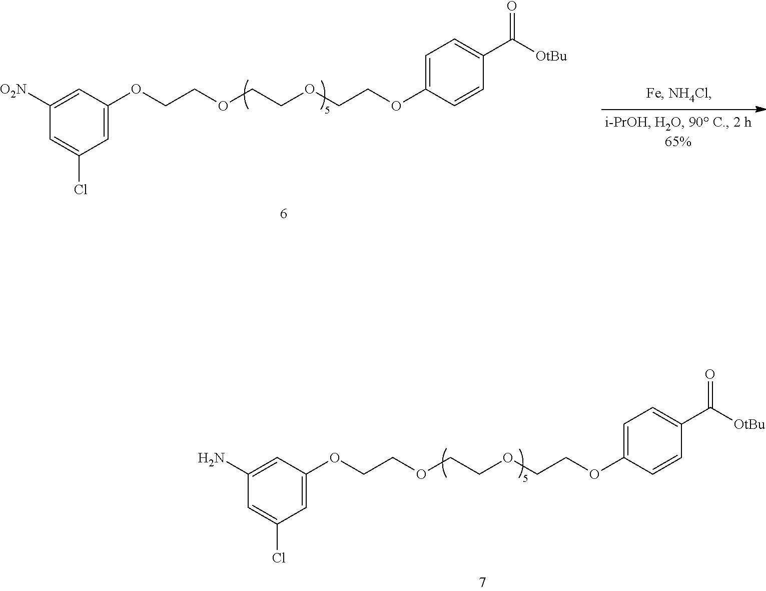

To a solution of compound 6 (1.0 g, 1.52 mmol, 1.0 equiv) in i-PrOH (10 mL) was added Fe (425 mg, 7.61 mmol, 5.0 equiv) and NH₄Cl (405 mg, 7.6 mmol, 5.0 equiv) in H₂O (1 mL). The mixture was stirred at 90° C. for 2 h. The reaction mixture was filtered and concentrated under reduced pressure to give a residue. The residue was purified by prep-HPLC (column: Waters Xbridge C18 150*50 mm*10 μm; mobile phase: [water (10 mM NH₄HCO₃)-ACN]; B %: 47%-77%). Compound 7 (620 mg, 987 μmol, 65% yield) was obtained as a yellow oil. ¹HNMR (400 MHz, CDCl₃): δ 7.92 (d, J=8.2 Hz, 2H), 6.90 (d, J=8.6 Hz, 2H), 6.48-6.30 (m, 3H), 4.16 (m, 2H), 4.09 (m, 2H), 3.89 (t, J=4.2 Hz, 2H), 3.80 (t, J=4.6 Hz, 2H), 3.77-3.72 (m, 2H), 3.72-3.62 (m, 18H), 1.59 (s, 9H). LC-MS: MS (ES⁺): RT=0.983 min, m/z=628.1 [M+H⁺].

### 5. Preparation of Compound 9

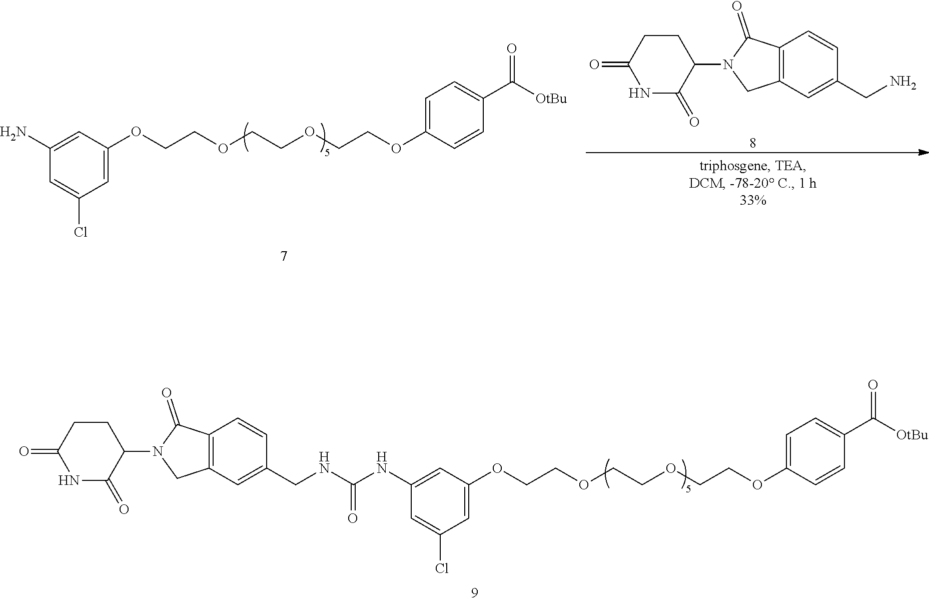

To a solution of triphosgene (50 mg, 168 μmol, 0.52 equiv) in DCM (20 mL) was added TEA (145 mg, 1.44 mmol, 200 μL, 4.5 equiv) and compound 7 (200 mg, 318 μmol, 1.0 equiv) in DCM (20 mL) at −78° C., and then it was stirred for 0.5 h. Compound 8 (108 mg, 348 μmol, 1.1 equiv, HCl salt) was added at −78° C. The mixture was added and stirred at 20° C. for 0.5 h. The reaction mixture was poured into aq. NaHCO₃ (50 mL) at 0° C., and then extracted with DCM (50 mL). The combined organic layers were washed with brine (50 mL), dried over Na₂SO₄, filtered and concentrated under reduced pressure to give a residue. The residue was purified by prep-TLC (SiO₂, DCM:MeOH=10:1). Compound 9 (100 mg, 107 μmol, 33% yield) was obtained as a yellow solid. LC-MS: MS (ES⁺): RT=0.975 min, m/z=871.0 [M−55].

### 6. Preparation of Compound 10

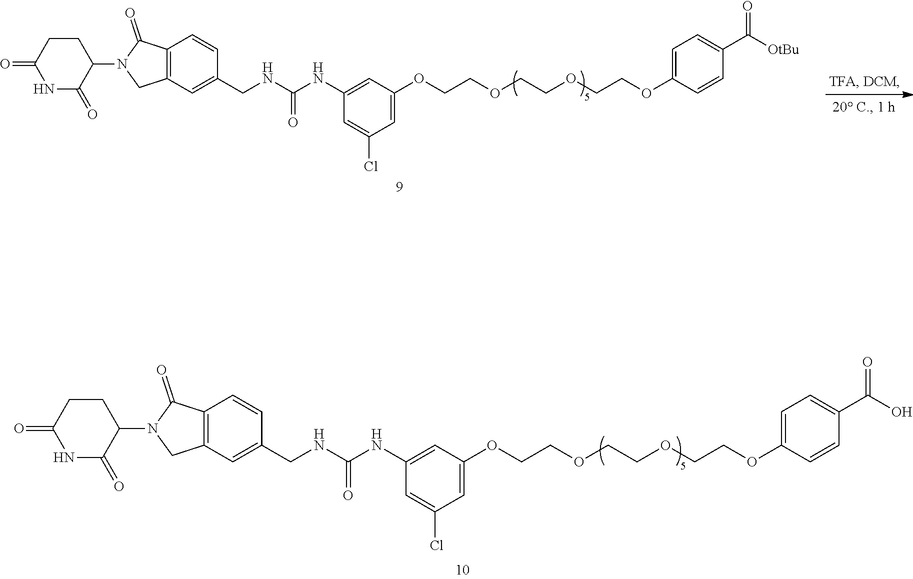

To a solution of compound 9 (100 mg, 107 μmol, 1.0 equiv) in DCM (3 mL) was added TFA (1 mL). The mixture was stirred at 20° C. for 0.5 h. The reaction mixture was concentrated under reduced pressure to give a residue. Compound 10 (90 mg, crude) was obtained as a yellow oil. LC-MS: MS (ES⁺): RT=0.876 min, m/z=871.0 [M+1].

### 7. Preparation of I-19

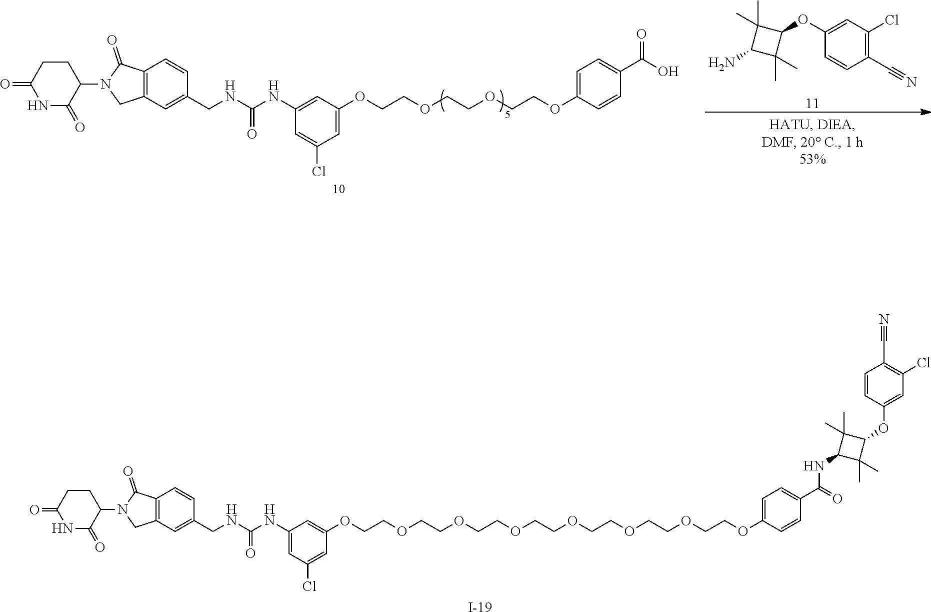

To a solution of compound 10 (90 mg, 103.29 μmol, 1 equiv) and compound 11 (60 mg, 152 μmol, 1.4 equiv, TFA) in DMF (1 mL) was added DIEA (74 mg, 574 μmol, 0.1 mL, 5.5 equiv) and HATU (78 mg, 205 μmol, 2 equiv). The mixture was stirred at 20° C. for 0.5 h. The reaction mixture was quenched by addition H₂O (0.01 mL), and then filtered and concentrated under reduced pressure to give a residue. The residue was purified by prep-HPLC (column: Phenomenex Gemini-NX C18 75*30 mm*3 μm; mobile phase: [water (0.225% FA)-ACN]; B %: 52%-82%, 7 min) to give compound N-((1r,3r)-3-(3-chloro-4-cyanophenoxy)-2,2,4,4-tetramethylcyclobutyl)-4-((20-(3-chloro-5-(3-((2-(2,6-dioxopiperidin-3-yl)-1-oxoisoindolin-5-yl)methyl)ureido)phenoxy)-3,6,9,12,15,18-hexaoxaicosyl)oxy)benzamide (62 mg, 55 μmol, 53% yield, 100% purity) as a off-white solid. ¹HNMR (400 MHz, CD₃OD): δ 7.82-7.69 (m, 4H), 7.54 (s, 1H), 7.48 (d, J=8.3 Hz, 1H), 7.12 (d, J=2.4 Hz, 1H), 7.07 (t, J=1.8 Hz, 1H), 7.04-6.94 (m, 4H), 6.58 (t, J=2.0 Hz, 1H), 5.18-5.09 (m, 1H), 4.54-4.40 (m, 4H), 4.28 (s, 1H), 4.21-4.11 (m, 3H), 4.10-4.05 (m, 2H), 3.86-3.78 (m, 4H), 3.69-3.59 (m, 20H), 2.97-2.83 (m, 1H), 2.81-2.73 (m, 1H), 2.55-2.41 (m, 1H), 2.22-2.11 (m, 1H), 1.28 (s, 6H), 1.22 (s, 6H). LC-MS: MS (ES⁺): RT=2.621 min, m/z=1130.6, 1132.6 [M+H⁺]; LCMS method: LC-MS METHOD 25.M

### Example 20—Preparation of Compound I-20

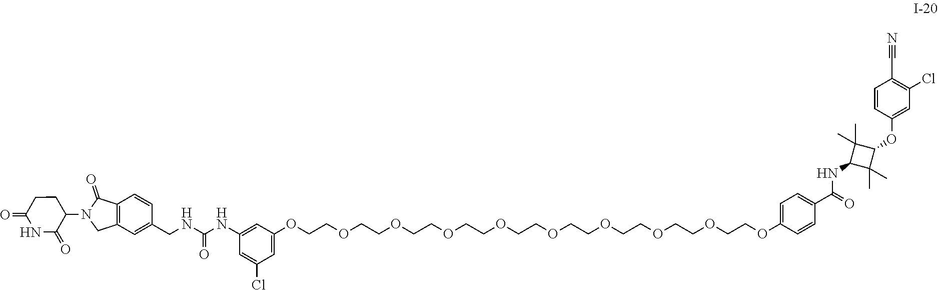 [SMILES: CC1(C)[C@H](NC(=O)c2ccc(OCCOCCOCCOCCOCCOCCOCCOCCOCCOc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1]

The title compound was prepared according to the following procedures.

### 1. Preparation of Compound 2

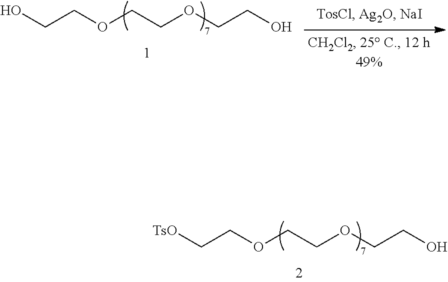

To a solution of 2-[2-[2-[2-[2-[2-[2-[2-(2-hydroxyethoxy)ethoxy]ethoxy]ethoxy]ethoxy]ethoxy]ethoxy]ethoxy]ethanol (4.50 g, 10.9 mmol, 1.0 equiv) in CH₂Cl₂ (100 mL) was added Ag₂O (3.77 g, 16.3 mmol, 1.5 equiv), NaI (1.79 g, 11.9 mmol, 1.1 equiv) and TosCl (2.07 g, 10.9 mmol, 1.0 equiv), and then it was stirred at 25° C. for 12 h. The reaction mixture was filtered and the filtrate was concentrated to afford crude product. The residue was purified by silica chromatography (EtOAc:MeOH=1:0-10:1) to afford 2-[2-[2-[2-[2-[2-[2-[2-(2-hydroxyethoxy) ethoxy]ethoxy]ethoxy]ethoxy]ethoxy]ethoxy]ethoxy]ethyl 4-methylbenzenesulfonate (3.0 g, 5.3 mmol, 49% yield) as a colorless oil. ¹H NMR: (400 MHz, CDCl₃) δ 7.80 (d, J=8.3 Hz, 2H), 7.34 (d, J=8.0 Hz, 2H), 4.20-4.13 (m, 2H), 3.77-3.54 (m, 34H), 2.49 (brs, 1H), 2.45 (s, 3H). LC-MS: MS (ES⁺): RT=0.779 min, m/z=569.2 [M+H⁺].

### 2. Preparation of Compound 4

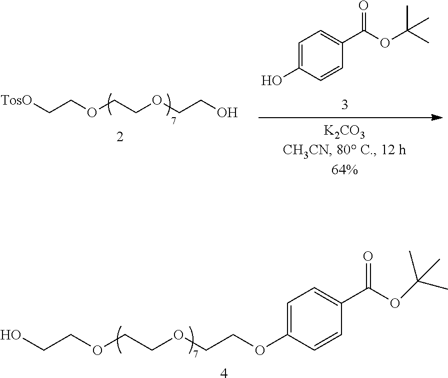

To a solution of 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (3.0 g, 5.3 mmol, 1.0 equiv) and tert-butyl 4-hydroxybenzoate (1.02 g, 5.28 mmol, 1.0 equiv) in CH₃CN (20 mL) was added K₂CO₃ (2.19 g, 15.8 mmol, 3.0 equiv), and then it was stirred at 80° C. for 12 h. The reaction mixture was filtered and the filtrate was concentrated to afford crude product The residue was purified by silica chromatography (EtOAc:MeOH=1:0-10:1) to afford tert-butyl 4-[2-[2-[2-[2-[2-[2-[2-[2-(2-hydroxyethoxy)ethoxy]ethoxy]ethoxy]ethoxy]ethoxy]ethoxy]ethoxy]ethoxy]benzoate (2.0 g, 3.4 mmol, 64% yield) as a light yellow oil. ¹H NMR: (400 MHz, CDCl₃) δ 7.93 (d, J=8.9 Hz, 2H), 6.91 (d, J=8.9 Hz, 2H), 4.22-4.14 (m, 2H), 3.92-3.85 (m, 2H), 3.76-3.57 (m, 32H), 1.58 (s, 9H). LC-MS: MS (ES⁺): RT=0.839 min, m/z=608.3 [M+H₃O]⁺.

### 3. Preparation of Compound 5

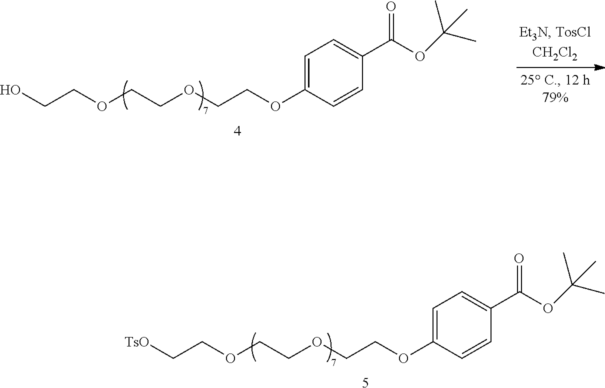

To a solution of tert-butyl 4-[2-[2-[2-[2-[2-[2-[2-[2-(2-hydroxyethoxy)ethoxy]ethoxy]ethoxy]ethoxy]ethoxy]ethoxy]ethoxy]ethoxy]benzoate (2.0 g, 3.4 mmol, 1.0 equiv) and Et₃N (685 mg, 6.77 mmol, 942 μL, 2.0 equiv) in CH₂Cl₂ (20 mL) was added TosCl (775 mg, 4.06 mmol, 1.2 equiv), and then it was stirred at 25° C. for 12 h. The reaction mixture was concentrated to afford crude product. The residue was purified by silica chromatography (EtOAc:MeOH=1:0-10:1) to afford tert-butyl 4-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethoxy]benzoate (2.0 g, 2.7 mmol, 79% yield) as a yellow oil. ¹H NMR (400 MHz, CDCl₃) δ 7.93 (d, J=8.9 Hz, 2H), 7.80 (d, J=8.3 Hz, 2H), 7.35 (d, J=8.1 Hz, 2H), 6.91 (d, J=8.8 Hz, 2H), 4.17 (q, J=4.8 Hz, 4H), 3.95-3.83 (m, 2H), 3.79-3.57 (m, 30H), 2.45 (s, 3H), 1.58 (s, 9H). LC-MS: MS (ES⁺): RT=0.962 min, m/z=689.1 [M−55]⁺.

### 4. Preparation of Compound 7

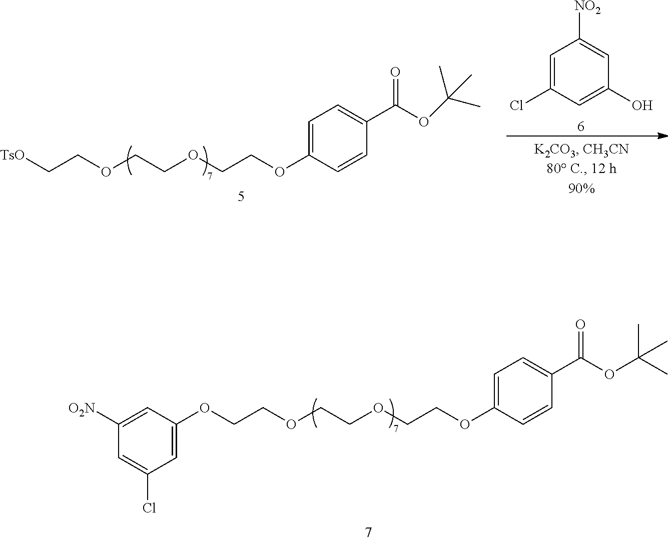

To a solution of tert-butyl 4-[2-[2-[2-[2-[2-[2-[2-[2-[2-(p-tolylsulfonyloxy) ethoxy]ethoxy]ethoxy]ethoxy]ethoxy]ethoxy]ethoxy]ethoxy]ethoxy]benzoate (500 mg, 671 μmol, 1.0 equiv) and 3-chloro-5-nitro-phenol (117 mg, 671 μmol, 1.0 equiv) in CH₃CN (5 mL) was added K₂CO₃ (278 mg, 2.01 mmol, 3.0 equiv), and then it was stirred at 80° C. for 12 h. The reaction mixture was filtered and the filtrate was concentrated to afford crude product. The residue was purified by silica chromatography (EtOAc:MeOH=1:0-10:1) to afford tert-butyl 4-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]benzoate (450 mg, 603 μmol, 90% yield) as a colorless oil. ¹H NMR (400 MHz, CDCl₃) δ 7.85 (d, J=8.6 Hz, 2H), 7.74 (s, 1H), 7.65-7.52 (m, 1H), 7.19 (d, J=6.0 Hz, 1H), 6.83 (d, J=8.6 Hz, 2H), 4.12 (m, 4H), 3.80 (d, J=3.5 Hz, 4H), 3.69-3.49 (m, 28H), 1.51 (s, 9H). LC-MS: MS (ES+): RT=1.087 min, m/z=690.2 [M-55]+.

### 5. Preparation of 8

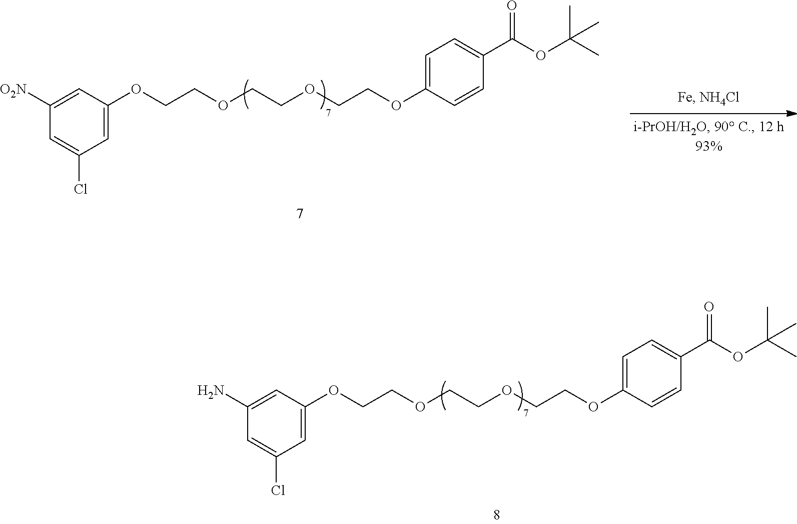

To a solution of tert-butyl 4-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]benzoate (450 mg, 603 μmol, 1.0 equiv) in i-PrOH (18 mL) was added NH₄Cl (645 mg, 12.1 mmol, 20.0 equiv) in H₂O (2 mL), and then Fe (337 mg, 6.03 mmol, 10.0 equiv) was added and it was stirred for 2 h at 90° C. The reaction mixture was filtered and the filter cake was washed with EtOAc (2×20 mL). The filtrate was added 50 mL water and then extracted with EtOAc (2×20 mL). The organic layers were dried over anhydrous Na₂SO₄, filtered and concentrated to afford crude product. The residue was purified by silica chromatography (EtOAc:MeOH=1:0-10:1) to afford tert-butyl 4-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]benzoate (400 mg, 558 μmol, 93% yield) as a yellow oil. ¹H NMR: (400 MHz, CDCl₃) δ 7.92 (d, J=8.9 Hz, 2H), 6.90 (d, J=8.9 Hz, 2H), 6.79-6.27 (m, 3H), 4.19-4.14 (m, 2H), 4.10-4.04 (m, 2H), 3.90-3.85 (m, 2H), 3.83-3.78 (m, 2H), 3.76-3.61 (m, 26H), 1.58 (s, 9H). LC-MS: MS (ES⁺): RT=0.997 min, m/z=716.3 [M+H⁺].

### 6. Preparation of 10

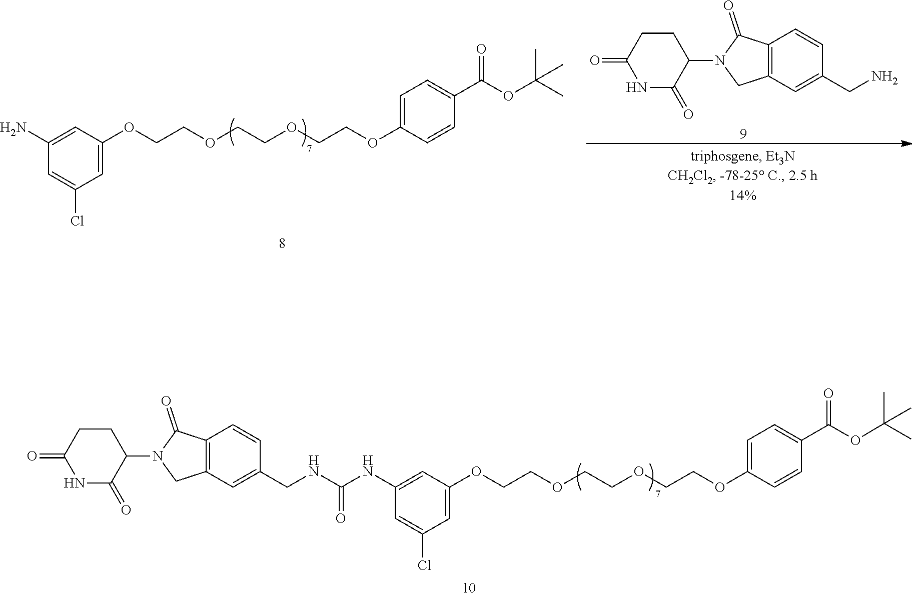

To a solution of triphosgene (300 mg, 101 μmol, 0.36 equiv) and Et₃N (85 mg, 0.84 mmol, 0.12 mL, 3.0 equiv) in CH₂Cl₂ (5 mL) was added tert-butyl 4-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]benzoate (200 mg, 279 μmol, 1.0 equiv) in CH₂Cl₂ (2 mL) at −78° C., and then it was stirred for 0.5 h. 3-[5-(aminomethyl)-1-oxo-isoindolin-2-yl]piperidine-2,6-dione (104 mg, 335 μmol, 1.2 equiv, HCl salt) was added to the mixture and then it was slowly warmed to 25° C. and stirred for 2 h. The reaction mixture was slowly poured into 50 mL sat. NaHCO₃ and 100 mL water. After extracted with EtOAc (2×50 mL), the organic layers were dried over anhydrous Na₂SO₄, filtered and concentrated to afford crude product. The residue was purified by prep-TLC (CH₂Cl₂:MeOH=10:1) to afford tert-butyl 4-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]benzoate (40 mg, 39 μmol, 14% yield) as a yellow gum. LC-MS: MS (ES⁺): RT=0.985 min, m/z=959.0 [M−55]⁺.

### 7. Preparation of 11

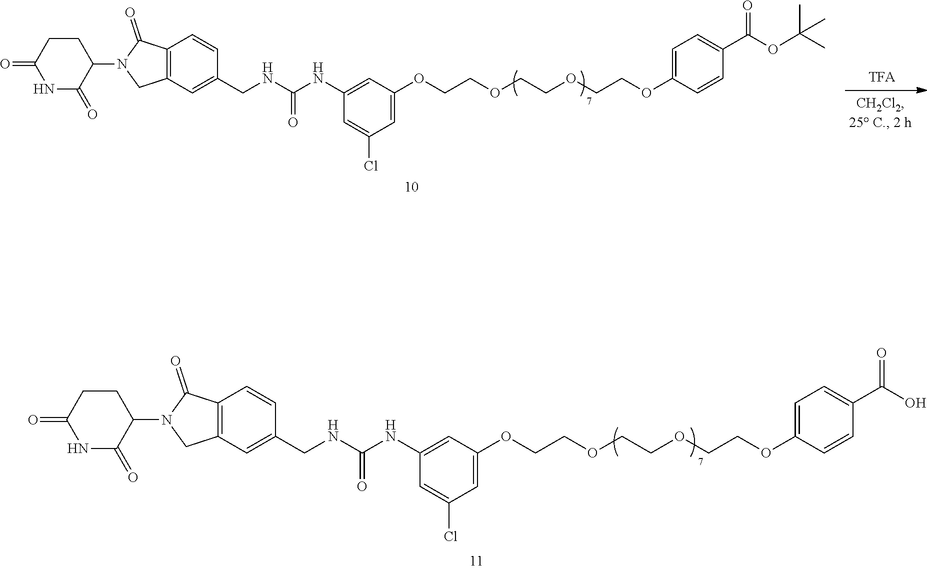

To a solution of tert-butyl 4-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]benzoate (40 mg, 39 μmol, 1.0 equiv) in CH₂Cl₂ (2 mL) was added TFA (1 mL), and then it was stirred at 25° C. for 2 h. The reaction was concentrated to afford crude product. 4-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]benzoic acid (38 mg, crude) was obtained as a yellow gum. LC-MS: MS (ES⁺): RT=0.865 min, m/z=959.4 [M+H⁺].

### 8. Preparation of I-20

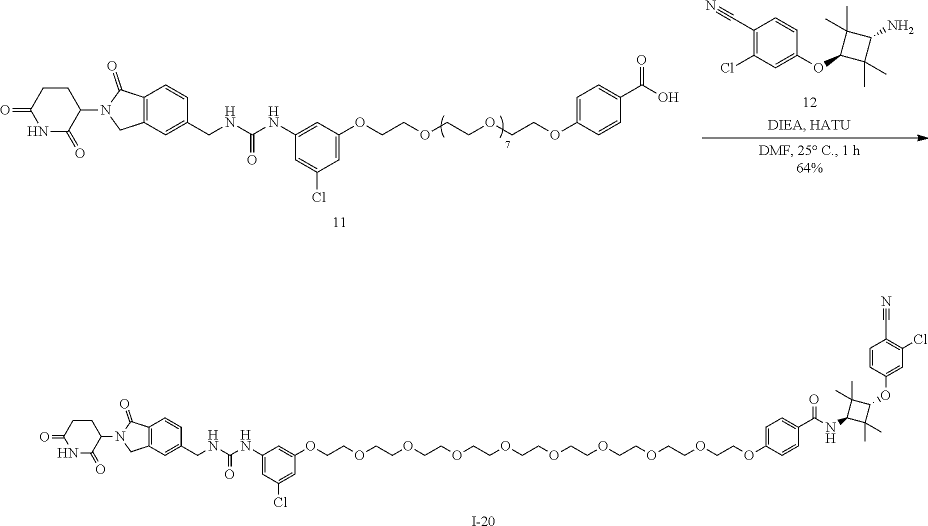

To a solution of 4-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]benzoic acid (38 mg, 40 μmol, 1.0 equiv) and 4-(3-amino-2,2,4,4-tetramethyl-cyclobutoxy)-2-chloro-benzonitrile (23 mg, 59 μmol, 1.5 equiv, TFA salt) in DMF (2 mL) was added DIEA (10 mg, 79 μmol, 14 μL, 2.0 equiv) and HATU (18 mg, 48 μmol, 1.2 equiv), and then it was stirred at 25° C. for 1 h. The reaction mixture was quenched by 0.1 mL water. The residue was purified by prep-HPLC (column: Waters Xbridge 150*25 mm*5 um; mobile phase: [water(10 mM NH₄HCO₃)-ACN]; B %: 50%-80%, 9 min) to afford N-[3-(3-chloro-4-cyano-phenoxy)-2,2,4,4-tetramethyl-cyclobutyl]-4-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]benzamide (31 mg, 25 μmol, 64% yield) as a colorless gum. ¹H NMR: (400 MHz, CD₃OD) δ 7.79 (d, J=8.7 Hz, 2H), 7.75 (d, J=7.9 Hz, 1H), 7.71 (d, J=8.7 Hz, 1H), 7.54 (s, 1H), 7.48 (d, J=7.9 Hz, 1H), 7.12 (d, J=2.3 Hz, 1H), 7.07 (t, J=1.7 Hz, 1H), 7.03-6.93 (m, 4H), 6.60-6.55 (m, 1H), 5.13 (m, 1H), 4.52-4.39 (m, 4H), 4.28 (s, 1H), 4.19-4.15 (m, 2H), 4.13 (s, 1H), 4.11-4.05 (m, 2H), 3.86-3.82 (m, 2H), 3.82-3.78 (m, 2H), 3.70-3.66 (m, 4H), 3.64 (m, 4H), 3.62-3.54 (m, 20H), 2.96-2.82 (m, 1H), 2.82-2.70 (m, 1H), 2.47 (m, 1H), 2.15 (m, 1H), 1.28 (s, 6H), 1.22 (s, 6H). LC-MS: MS (ES⁺): RT=2.783 min, m/z=610.5 [M/2+H⁺], LCMS method: LC-MS METHOD 25.

### Example 21—Preparation of Compound I-21

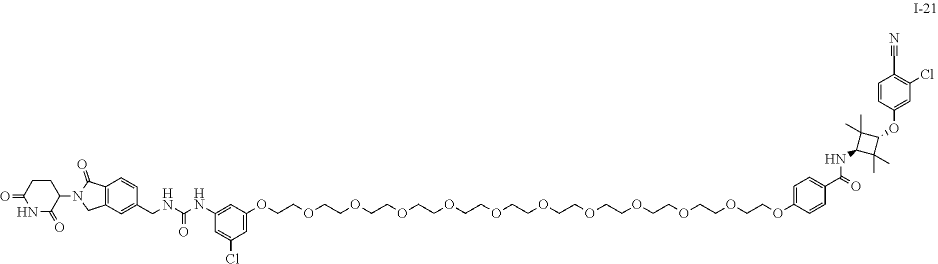 [SMILES: CC1(C)[C@H](NC(=O)c2ccc(OCCOCCOCCOCCOCCOCCOCCOCCOCCOCCOCCOc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1]

The title compound was prepared according to the following procedures.

### 1. Preparation of Compound 2

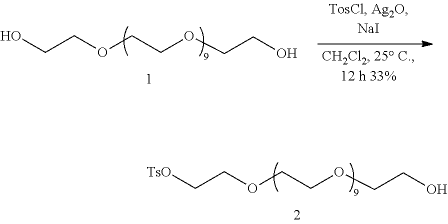

To a solution of 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethanol (6.00 g, 11.9 mmol, 1.0 equiv) in CH₂Cl₂ (100 mL) was added Ag₂O (4.15 g, 17.9 mmol, 1.5 equiv), NaI (1.97 g, 13.1 mmol, 1.1 equiv) and TosCl (2.28 g, 11.9 mmol, 1.0 equiv), and then it was stirred at 25° C. for 12 h. The reaction mixture was filtered and the filtrate was concentrated to afford crude product. The residue was purified by silica chromatography (EtOAc:MeOH=1:0-10:1) to afford 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl 4-methylbenzenesulfonate (2.60 g, 3.96 mmol, 33% yield). ¹H NMR: (400 MHz, CDCl₃) δ 7.80 (d, J=8.3 Hz, 2H), 7.35 (d, J=8.1 Hz, 2H), 4.21-4.13 (m, 2H), 3.80-3.55 (m, 42H), 2.45 (s, 3H), 2.33 (brs, 1H). LC-MS: MS (ES⁺): RT=0.772 min, m/z=657.1 [M+H⁺].

### 2. Preparation of Compound 4

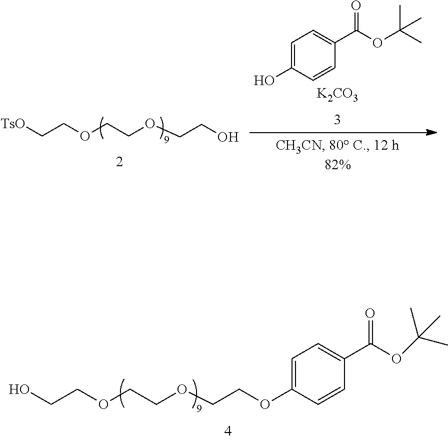

To a solution of 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethyl 4-methylbenzenesulfonate (2.60 g, 3.96 mmol, 1.0 equiv) and tert-butyl 4-hydroxybenzoate (769 mg, 3.96 mmol, 1.0 equiv) in CH₃CN (20 mL) was added K₂CO₃ (1.64 g, 11.9 mmol, 3.0 equiv), and then it was stirred at 80° C. for 12 h. The reaction mixture was filtered and the filtrate was concentrated to afford crude product. The residue was purified by silica chromatography (EtOAc:MeOH=1:0-10:1) to afford tert-butyl 4-[2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethoxy]benzoate (2.20 g, 3.24 mmol, 82% yield) as a light yellow oil. ¹H NMR: (400 MHz, CDCl₃) δ 7.93 (d, J=8.9 Hz, 2H), 6.91 (d, J=8.8 Hz, 2H), 4.17 (t, J=4.8 Hz, 2H), 3.88 (t, J=4.8 Hz, 2H), 3.76-3.59 (m, 38H), 1.58 (s, 9H). LC-MS: MS (ES⁺): RT=0.849 min, m/z=623.2 [M−55]⁺.

### 3. Preparation of Compound 5

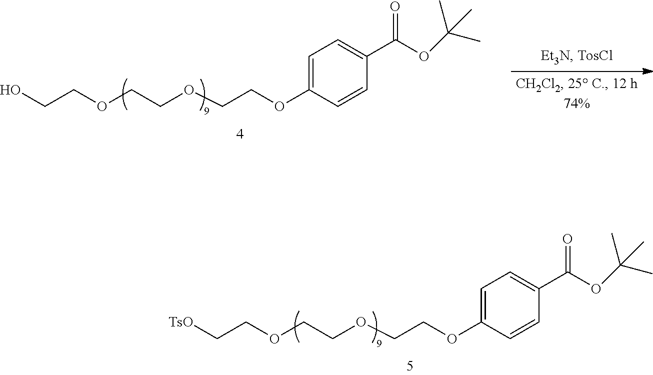

To a solution of tert-butyl 4-[2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]ethoxy]benzoate (2.20 g, 3.24 mmol, 1.0 equiv) and Et₃N (656 mg, 6.48 mmol, 902 μL, 2.0 equiv) in CH₂Cl₂ (20 mL) was added TosCl (741 mg, 3.89 mmol, 1.2 equiv), and then it was stirred at 25° C. for 12 h. The reaction mixture was concentrated to afford crude product. The residue was purified by silica chromatography (EtOAc:MeOH=1:0-10:1) to afford tert-butyl 4-[2-[2-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethoxy]ethoxy]ethoxy]benzoate (2.0 g, 2.4 mmol, 74% yield) as a yellow oil. ¹H NMR: (400 MHz, CDCl₃) δ 7.93 (d, J=8.9 Hz, 2H), 7.80 (d, J=8.3 Hz, 2H), 7.35 (d, J=8.0 Hz, 2H), 6.91 (d, J=8.8 Hz, 2H), 4.22-4.07 (m, 4H), 3.93-3.85 (m, 2H), 3.77-3.55 (m, 38H), 2.45 (s, 3H), 1.59 (s, 9H). LC-MS: MS (ES⁺): RT=0.950 min, m/z=776.9 [M−55]⁺.

### 4. Preparation of Compound 7

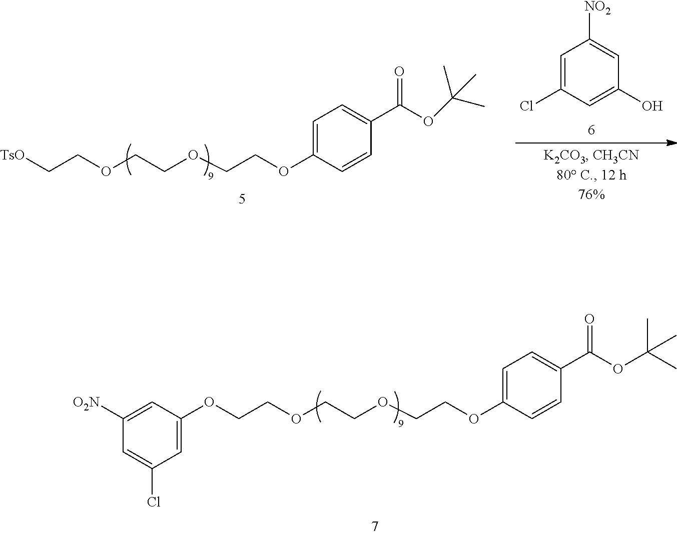

To a solution of tert-butyl 4-[2-[2-[2-[2-[2-[2-[2-[2-[2-[2-[2-(p-tolylsulfonyloxy) ethoxy]ethoxy]ethoxy]ethoxy]ethoxy]ethoxy]ethoxy]ethoxy]ethoxy]ethoxy]ethoxy]benzoate (500 mg, 600 μmol, 1.0 equiv) and 3-chloro-5-nitro-phenol (104 mg, 600 μmol, 1.0 equiv) in CH₃CN (5 mL) was added K₂CO₃ (249 mg, 1.80 mmol, 3.0 equiv), and then it was stirred at 80° C. for 12 h. The reaction mixture was filtered and the filtrate was concentrated to afford crude product. The residue was purified by silica chromatography (EtOAc:MeOH=1:0-10:1) to afford tert-butyl 4-[2-[2-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]ethoxy]ethoxy]benzoate (380 mg, 455 μmol, 76% yield) as a colorless oil. ¹H NMR: (400 MHz, CDCl₃) δ 7.93 (d, J=8.7 Hz, 2H), 7.82 (s, 1H), 7.68 (s, 1H), 7.27-7.24 (m, 1H), 6.91 (d, J=8.8 Hz, 2H), 4.19 (m, 4H), 3.94-3.84 (m, 4H), 3.78-3.57 (m, 36H), 1.59 (s, 9H). LC-MS: MS (ES⁺): m/z=778.2 [M−55]⁺.

### 5. Preparation of Compound 8

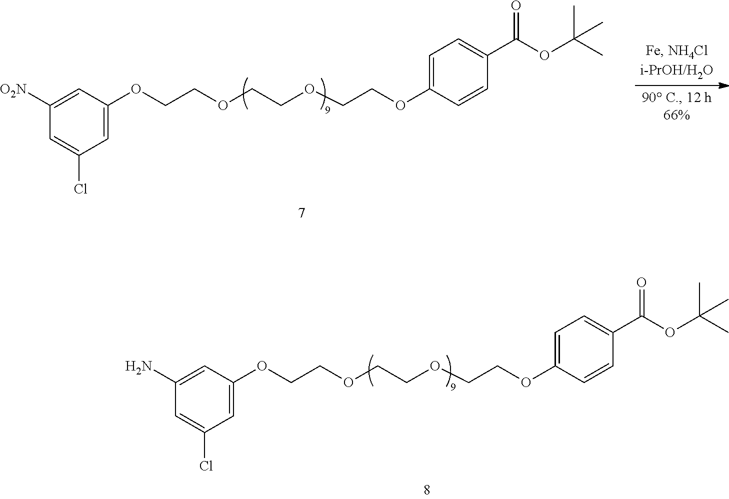

To a solution of tert-butyl 4-[2-[2-[2-[2-[2-[2-[2-[2-[2-[2-[2-(3-chloro-5-nitro-phenoxy)ethoxy]ethoxy]ethoxy]ethoxy]ethoxy]ethoxy]ethoxy]ethoxy]ethoxy]ethoxy]ethoxy]benzoate (380 mg, 455 μmol, 1.0 equiv) in i-PrOH (18 mL) was added NH₄Cl (487 mg, 9.11 mmol, 20.0 equiv) in H₂O (2 mL), and then Fe (254 mg, 4.55 mmol, 10.0 equiv) was added and stirred for 2 h at 90° C. The reaction mixture was filtered and the filter cake was washed with EtOAc (2×20 mL). The filtrate was added 50 mL water and then extracted with EtOAc (2×20 mL). The organic layers were dried over anhydrous Na₂SO₄, filtered and concentrated to afford crude product. The residue was purified by silica chromatography (EtOAc:MeOH=1:0-10:1) to afford tert-butyl 4-[2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]ethoxy]ethoxy]benzoate (240 mg, 298 μmol, 66% yield) as a yellow oil. LC-MS: MS (ES⁺): m/z=804.4 [M+H⁺].

### 6. Preparation of 10

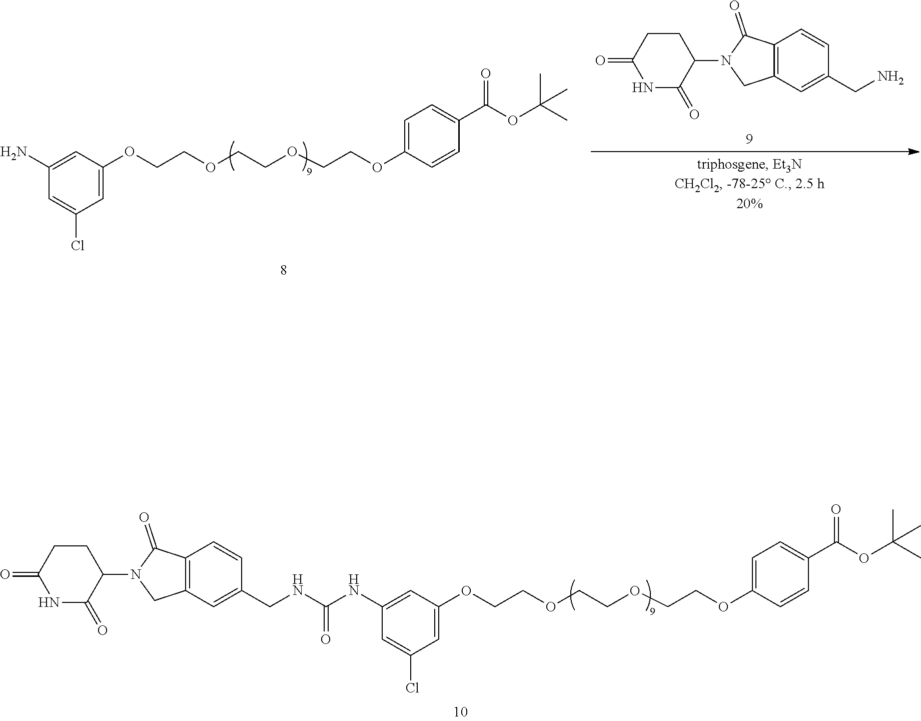

To a solution of Et₃N (91 mg, 0.90 mmol, 120 μL, 3.0 equiv) and triphosgene (44 mg, 0.15 mmol, 0.5 equiv) in CH₂Cl₂ (5 mL) was added tert-butyl 4-[2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]ethoxy]ethoxy]benzoate (240 mg, 298 μmol, 1.0 equiv) in CH₂Cl₂ (2 mL) at −78° C., and then it was stirred for 0.5 h. 3-[5-(aminomethyl)-1-oxo-isoindolin-2-yl]piperidine-2,6-dione (111 mg, 358 μmol, 1.2 equiv, HCl salt) was added to the mixture and then it was slowly warmed to 25° C. and stirred for 2 h. The reaction mixture was slowly poured into 50 mL sat·NaHCO₃ and 100 mL water. After extracted with EtOAc (2×50 mL), the organic layers were dried over anhydrous Na₂SO₄, filtered and concentrated to afford crude product. The residue was purified by prep-TLC (CH₂Cl₂:MeOH=10:1) to afford tert-butyl 4-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethoxy]benzoate (65 mg, 59 μmol, 20% yield) as a yellow gum. LC-MS: MS (ES⁺): RT=0.978 min, m/z=1103.5 [M+H⁺].

### 7. Preparation of 11

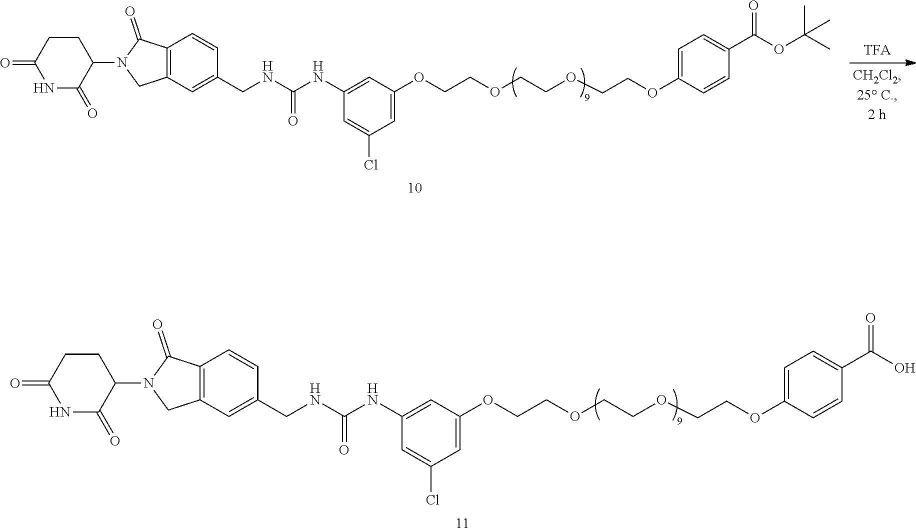

To a solution of tert-butyl 4-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethoxy]benzoate (65 mg, 59 μmol, 1.0 equiv) in CH₂Cl₂ (2 mL) was added TFA (1 mL), and then it was stirred at 25° C. for 2 h. The reaction was concentrated to afford 4-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethoxy]benzoic acid (62 mg, crude) as a yellow gum. LC-MS: MS (ES⁺): RT=0.865 min, m/z=1047.5 [M+H⁺].

### 8. Preparation of I-21

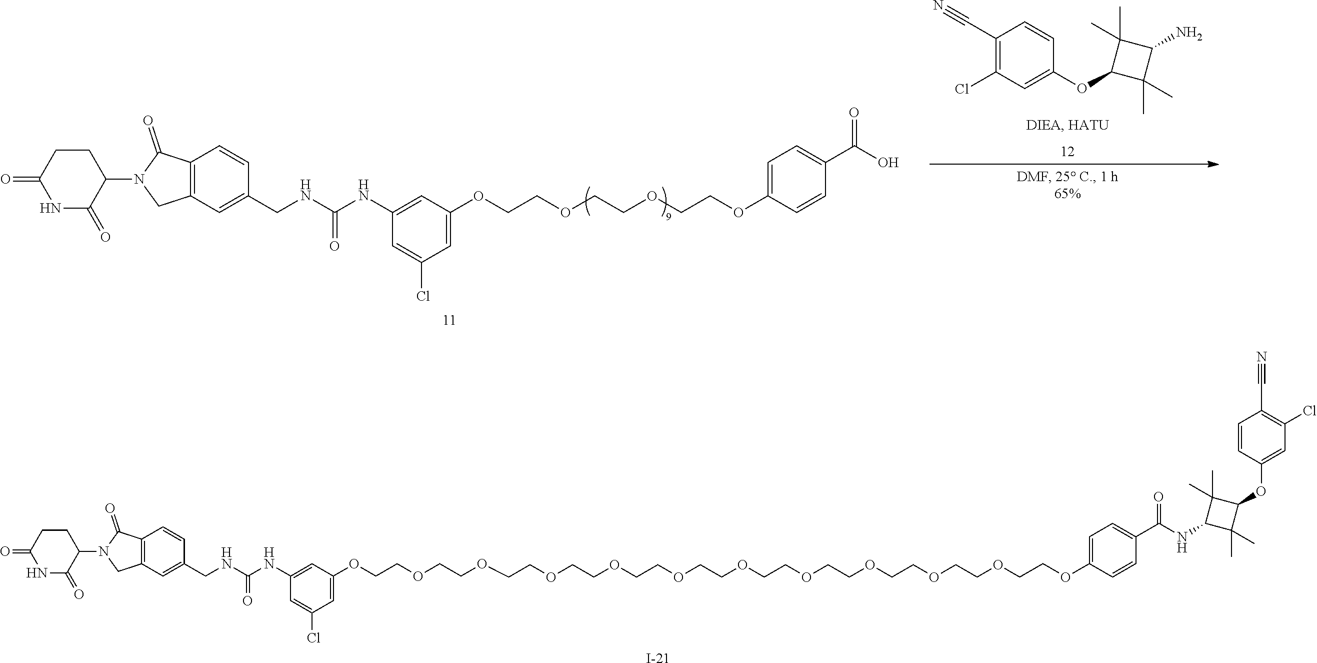

To a solution of 4-[2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]ethoxy]benzoic acid (62 mg, 59 μmol, 1.0 equiv) and 4-(3-amino-2,2,4,4-tetramethyl-cyclobutoxy)-2-chloro-benzonitrile (35 mg, 89 μmol, 1.5 equiv, TFA salt) in DMF (2 mL) was added DIEA (15 mg, 0.12 mmol, 21 μL, 2.0 equiv) and HATU (27 mg, 71 μmol, 1.2 equiv), and then it was stirred at 25° C. for 1 h. The reaction mixture was quenched by 0.1 mL water. The residue was purified by prep-HPLC (column: Waters Xbridge 150*25 mm*5 μm; mobile phase: [water (10 mM NH₄HCO₃)-ACN]; B %: 50%-80%, 9 min) to afford N-[3-(3-chloro-4-cyano-phenoxy)-2,2,4,4-tetramethyl-cyclobutyl]-4-[2-[2-[2-[2-[2-[2-[2-[2-[2-[2-[2-[3-chloro-5-[[2-(2,6-dioxo-3-piperidyl)-1-oxo-isoindolin- 5-yl]methylcarbamoyl amino]phenoxy]ethoxy]ethoxy]ethoxy]ethoxy]ethoxy]ethoxy]ethoxy]ethoxy]ethoxy]ethoxy]ethoxy]benzamide (50 mg, 38 μmol, 65% yield) as a colorless gum. ¹H NMR: (400 MHz, CD₃OD) δ 7.80 (d, J=8.8 Hz, 2H), 7.75 (d, J=7.8 Hz, 1H), 7.71 (d, J=8.8 Hz, 1H), 7.54 (s, 1H), 7.48 (d, J=7.9 Hz, 1H), 7.12 (d, J=2.2 Hz, 1H), 7.08 (s, 1H), 7.05-6.93 (m, 4H), 6.58 (t, J=1.7 Hz, 1H), 5.13 (m, 1H), 4.54-4.38 (m, 4H), 4.28 (s, 1H), 4.21-4.15 (m, 2H), 4.13 (s, 1H), 4.11-4.04 (m, 2H), 3.88-3.83 (m, 2H), 3.82-3.76 (m, 2H), 3.73-3.52 (m, 36H), 2.96-2.83 (m, 1H), 2.81-2.71 (m, 1H), 2.47 (m, 1H), 2.20-2.11 (m, 1H), 1.28 (s, 6H), 1.22 (s, 6H). LC-MS: MS (ES⁺): RT=2.781 min, m/z=655.1 [M/2+H⁺], LCMS method: LC-MS METHOD 25.

### Example 22—Preparation of Compound I-22

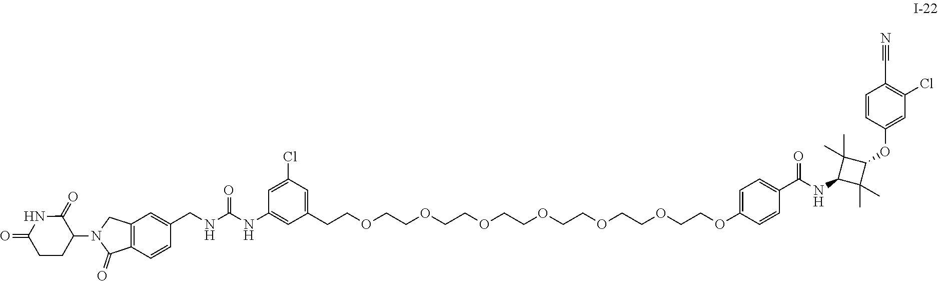 [SMILES: CC1(C)[C@H](NC(=O)c2ccc(OCCOCCOCCOCCOCCOCCOCCc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1]

The title compound was prepared according to the following procedures.

### 1. Preparation of Compound 2

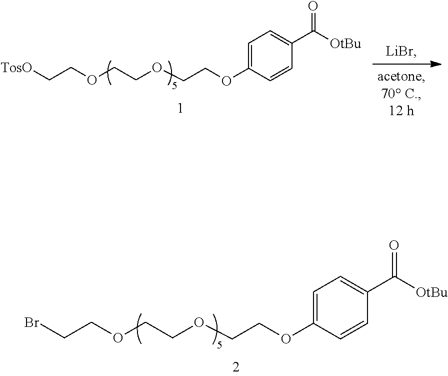

To a solution of compound 1 (2.0 g, 3.05 mmol, 1.0 equiv) in acetone (20 mL) was added LiBr (1.32 g, 15.23 mmol, 382 μL, 5.0 equiv). The mixture was stirred at 80° C. for 12 h. The reaction mixture was concentrated under reduced pressure to give crude compound 2 (1.8 g, crude). ¹HNMR (400 MHz, CDCl₃): δ 7.93 (d, J=8.9 Hz, 2H), 6.91 (d, J=8.9 Hz, 2H), 4.22-4.15 (m, 2H), 3.91-3.86 (m, 2H), 3.84-3.79 (m, 2H), 3.76-3.72 (m, 2H), 3.71-3.63 (m, 20H), 3.48 (t, J=6.3 Hz, 2H), 1.59 (s, 9H). LC-MS: MS (ES⁺): RT=1.000 min, m/z=584.0 [M+18]+.

### 2. Preparation of Compound 4

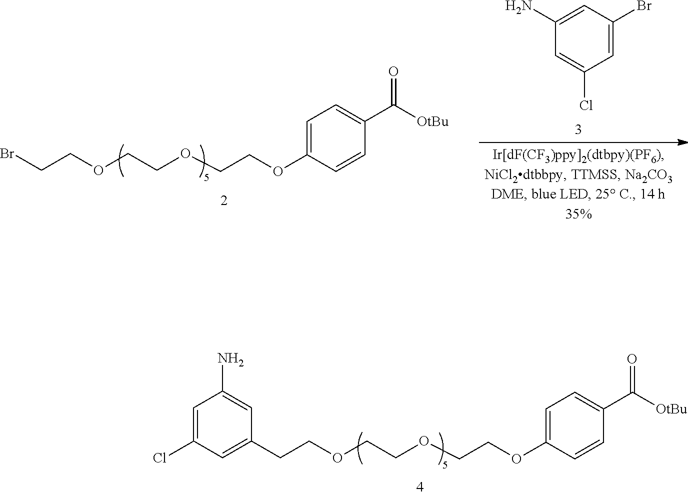

To a 40 mL vial equipped with a stir bar was added compound 2 (1.78 g, 3.15 mmol, 1.3 equiv), compound 3 (500 mg, 2.42 mmol, 1.0 equiv), Ir[dF(CF₃)ppy]₂(dtbpy)(PF₆) (27 mg, 24 μmol, 0.01 equiv), NiCl₂·dtbbpy (48 mg, 121 μmol, 0.05 equiv), TTMSS (602 mg, 2.42 mmol, 747 μL, 1.0 equiv), Na₂CO₃ (513 mg, 4.84 mmol, 2.0 equiv) in DME (40 mL). The vial was sealed and placed under nitrogen. The reaction was stirred and irradiated with a 34 W blue LED lamp (7 cm away), with cooling fan to keep the reaction temperature at 25° C. for 14 h. The reaction mixture was filtered and concentrated under reduced pressure to give a residue. The residue was purified by column chromatography (SiO₂, Petroleum ether/Ethyl acetate=1/1 to EA/MeOH=10/1). The residue was purified by prep-HPLC (column: Waters Xbridge C18 150*50 mm*10 μm; mobile phase: [water (10 mM NH₄HCO₃)-ACN]; B %: 48%-78%, 2 min). Compound 4 (520 mg, 849 μmol, 35% yield) was obtained as a yellow oil. ¹HNMR (400 MHz, CDCl₃): δ 7.92 (d, J=8.8 Hz, 2H), 6.90 (d, J=8.7 Hz, 2H), 6.73-6.53 (m, 3H), 4.20-4.14 (t, J=4.8 Hz, 2H), 3.92-3.86 (t, J=4.6 Hz, 2H), 3.78-3.72 (m, 2H), 3.70-3.59 (m, 20H), 2.77 (t, J=6.7 Hz, 2H), 1.59 (s, 9H). LC-MS: MS (ES⁺): RT=0.975 min, m/z=612.1 [M+H⁺].

### 3. Preparation of Compound 6

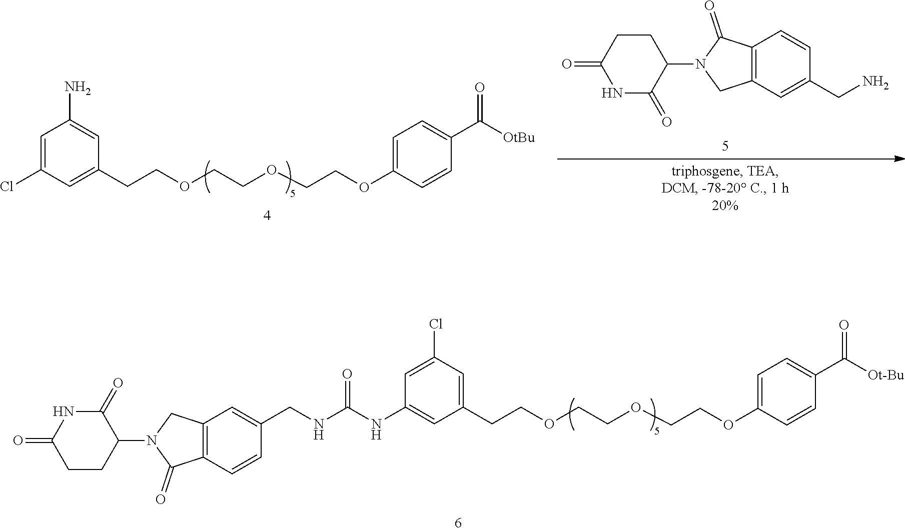

To a solution of triphosgene (50 mg, 168 μmol, 0.51 equiv) in DCM (20 mL) was added TEA (145 mg, 1.44 mmol, 0.2 mL, 4.4 equiv) and compound 4 (200 mg, 326 μmol, 1.0 equiv) in DCM (20 mL) at −78° C., and then it was stirred for 0.5 h. Compound 5 (98 mg, 359 μmol, 1.1 equiv) was added at −78° C. The mixture was added and stirred at 20° C. for 0.5 h. The reaction mixture was poured into aq. NaHCO₃ (50 mL) at 0° C., and then extracted with DCM (2×50 mL). The combined organic layers were washed with brine (50 mL), dried over Na₂SO₄, filtered and concentrated under reduced pressure to give a residue. The residue was purified by prep-TLC (SiO₂, DCM:MeOH=10:1). Compound 6 (60 mg, 65 μmol, 20% yield) was obtained as a yellow solid. LC-MS: MS (ES⁺): RT=0.983 min, m/z=855.0 [M−55]+.

### 4. Preparation of Compound 7

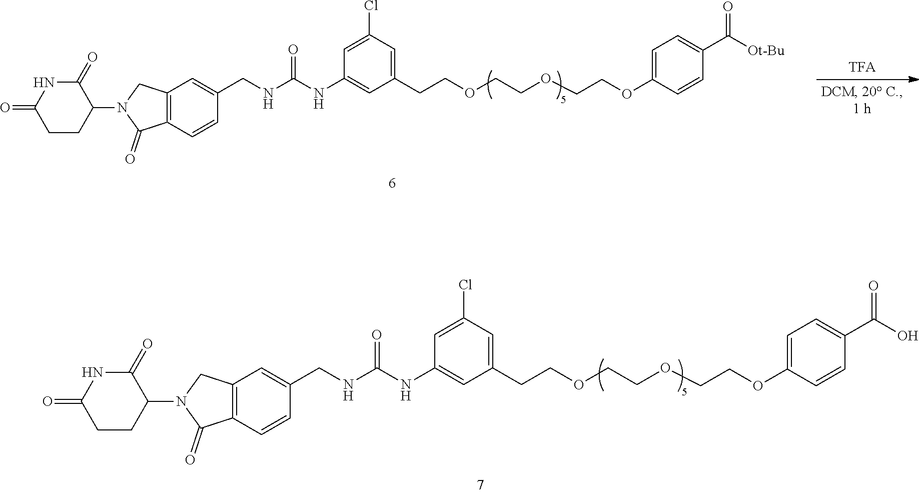

To a solution of compound 6 (60 mg, 65 μmol, 1.0 equiv) in DCM (3 mL) was added TFA (1 mL). The mixture was stirred at 20° C. for 0.5 h. The reaction mixture was concentrated under reduced pressure to give a residue. Compound 7 (50 mg, crude) was obtained as a yellow oil. LC-MS: MS (ES⁺): RT=0.883 min, m/z=855.0 [M+H⁺].

### 5. Preparation of I-22

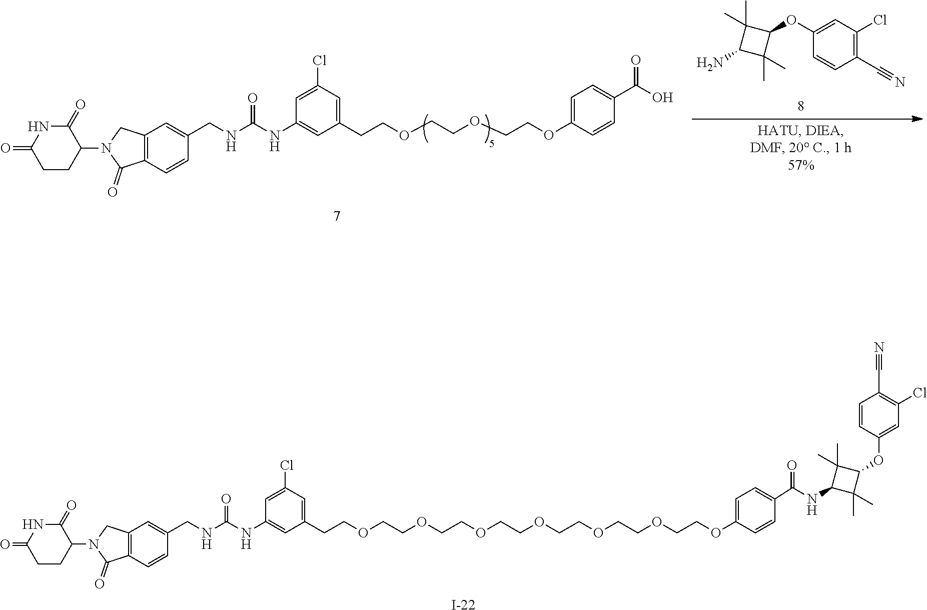

To a solution of compound 7 (50 mg, 58 μmol, 1.0 equiv) and compound 8 (35 mg, 89 μmol, 1.5 equiv, TFA salt) in DMF (1 mL) was added HATU (44 mg, 115 μmol, 2 equiv) and DIEA (44 mg, 344 μmol, 60 μL, 5.8 equiv). The mixture was stirred at 20° C. for 0.5 h. The reaction mixture was quenched by addition H₂O (0.1 mL). The reaction mixture was purified by prep-HPLC (column: Phenomenex Gemini-NX C18 75*30 mm*3 μm; mobile phase: [water (0.225% FA)-ACN]; B %: 52%-82%, 7 min) to give desired compound N-((1r,3r)-3-(3-chloro-4-cyanophenoxy)-2,2,4,4-tetramethylcyclobutyl)-4-((20-(3-chloro-5-(3-((2-(2,6-dioxopiperidin-3-yl)-1-oxoisoindolin-5-yl)methyl)ureido)phenyl)-3,6,9,12,15,18-hexaoxaicosyl)oxy)benzamide (37 mg, 33 μmol, 57% yield, 100% purity) as an off-white solid. ¹HNMR (400 MHz, CD₃OD): δ 7.83-7.69 (m, 4H), 7.55 (s, 1H), 7.51-7.47 (m, 1H), 7.45 (t, J=1.9 Hz, 1H), 7.15-7.09 (m, 2H), 7.03-6.94 (m, 3H), 6.88 (s, 1H), 5.19-5.09 (m, 1H), 4.55-4.39 (m, 4H), 4.28 (s, 1H), 4.21-4.10 (m, 3H), 3.88-3.80 (m, 2H), 3.72-3.52 (m, 22H), 2.96-2.84 (m, 1H), 2.83-2.73 (m, 3H), 2.55-2.40 (m, 1H), 2.22-2.10 (m, 1H), 1.28 (s, 6H), 1.22 (s, 6H). LC-MS: MS (ES⁺): RT=2.656 min, m/z=1114.7, 1116.6 [M+H⁺]; LCMS method: LC-MS METHOD 25.M.

### Example 23—Preparation of Compound I-23

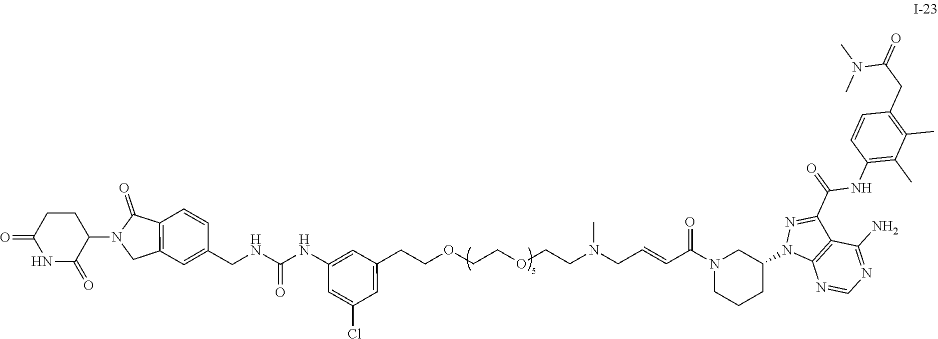

The title compound was prepared according to the following procedures.

### 1. Preparation of Compound 10

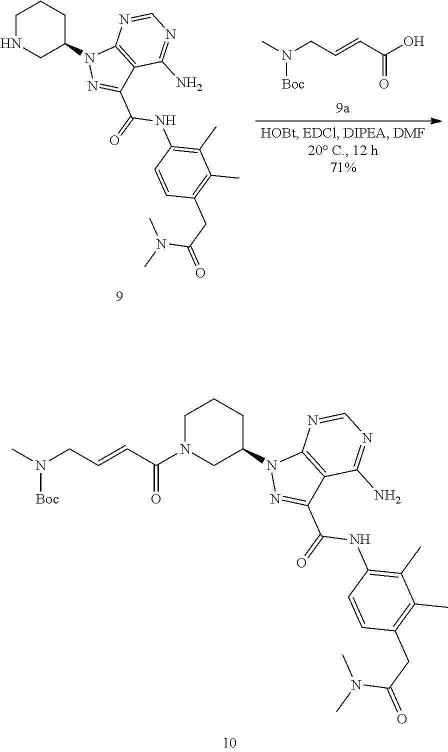

A mixture of 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (410 mg, 0.73 mmol, 1.0 equiv, TFA salt), (E)-4-[tert-butoxycarbonyl(methyl)amino]but-2-enoic acid (234 mg, 1.09 mmol, 1.5 equiv), HOBt (147 mg, 1.09 mmol, 1.5 equiv), EDCI (209 mg, 1.09 mmol, 1.5 equiv) and DIPEA (375 mg, 2.90 mmol, 4.0 equiv) in DMF (6 mL) was stirred at 20° C. for 12 h. The reaction mixture was diluted with water (30 mL) and the mixture was extracted with ethyl acetate (30 mL*3). The combined organic phase was washed with brine (50 mL), dried with anhydrous Na₂SO₄, filtered and concentrated in vacuo. The residue was purified by column chromatography on silica gel (Dichloromethane/Methanol=100/1 to 20/1) and prep-HPLC (column: Phenomenex luna C18 150*40 mm*15 μm; mobile phase: [water (0.225% FA)-ACN]; B %: 36%-46%, 10 min) to afford tert-butyl N-[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-N-methyl-carbamate (340 mg, 0.51 mmol, 71% yield) as a yellow oil. ¹H NMR (400 MHz, CDCl₃): δ 9.29 (s, 1H), 8.81 (s, 1H), 8.10 (s, 2H), 7.52 (d, 1H, J=8.0 Hz), 7.05 (d, 1H, J=8.4 Hz), 6.85-6.65 (m, 1H), 6.40-6.20 (m, 1H), 5.00-4.30 (m, 2H), 4.20-3.90 (m, 3H), 3.80-3.15 (m, 4H), 3.07 (s, 3H), 3.03 (s, 3H), 2.93-2.75 (m, 3H), 2.40-2.27 (m, 5H), 2.24 (s, 3H), 2.07-1.96 (m, 1H), 1.82-1.68 (m, 1H), 1.41 (s, 9H). LC-MS: MS (ES⁺): RT=0.767 min, m/z=648.5 [M+H⁺].

### 2. Preparation of Compound 8a

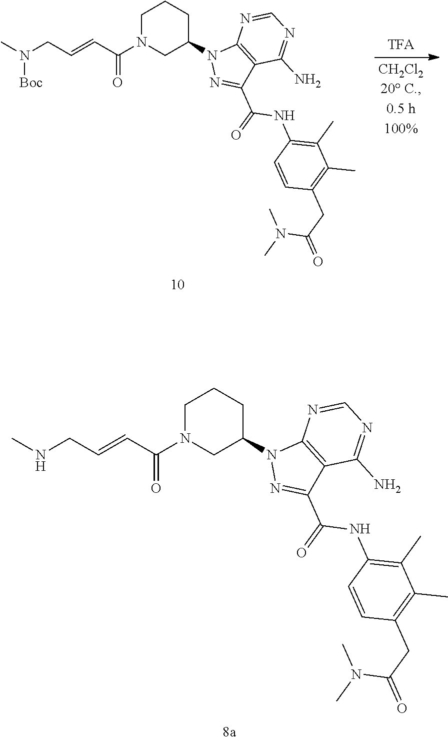

A mixture of tert-butyl N-[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-N-methyl-carbamate (28 mg, 0.04 mmol, 1 equiv) in DCM (1 mL) and TFA (0.5 mL) was stirred at 20° C. for 0.5 h. The mixture was concentrated in vacuo to afford 4-amino-N-[4-[2-(dimethylamino)-2-oxo-ethyl]-2,3-dimethyl-phenyl]-1-[(3R)-1-[(E)-4-(methylamino)but-2-enoyl]-3-piperidyl]pyrazolo[3,4-d]pyrimidine-3-carboxamide (28 mg, 0.04 mmol, 100% yield, TFA salt) as a light yellow oil. LC-MS: MS (ES⁺): RT=0.629 min, m/z=548.3 [M+H⁺].

### 3. Preparation of Compound 2

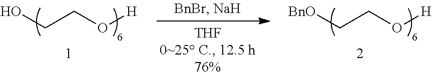

To a solution of 2-[2-[2-[2-[2-(2-hydroxyethoxy)ethoxy]ethoxy]ethoxy]ethoxy]ethanol (25.0 g, 88.6 mmol, 2.0 equiv) in THF (400 mL) was added NaH (1.95 g, 48.7 mmol, 60% purity, 1.1 equiv) at 0° C. The mixture was stirred at 0° C. for 0.5 h under N₂. To the mixture was added a solution of bromomethylbenzene (7.57 g, 44.3 mmol, 5.26 mL, 1.0 equiv) in THE (100 mL) dropwise at 0° C. The mixture was warmed to 25° C. and stirred for 12 h under N₂. The reaction mixture was quenched with sat. aq. NH₄Cl (100 mL) and the mixture was extracted with ethyl acetate (100 mL*3). The combined organic phase was washed with brine (100 mL), dried with anhydrous Na₂SO₄, filtered and concentrated in vacuo. The residue was purified by column chromatography on silica gel (Petroleum ether/Ethyl acetate=3/1-0/1 to Ethyl acetate/methanol=10/1) to afford 2-[2-[2-[2-[2-(2-benzyloxyethoxy)ethoxy]ethoxy]ethoxy]ethoxy]ethanol (12.6 g, 33.8 mmol, 76% yield) as a light yellow oil. ¹H NMR (400 MHz, DMSO-d₆) δ 7.42-7.21 (m, 5H), 4.56 (t, 1H, J=5.2 Hz), 4.49 (s, 2H), 3.57-3.54 (m, 4H), 3.52-3.46 (m, 18H), 3.43-3.38 (m, 2H).

### 4. Preparation of Compound 3

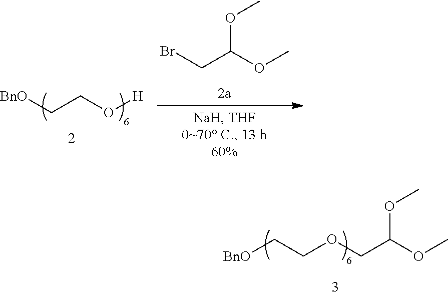

To a solution of 2-[2-[2-[2-[2-(2-benzyloxyethoxy)ethoxy]ethoxy]ethoxy]ethoxy]ethanol (12.6 g, 33.8 mmol, 1.0 equiv) in THE (300 mL) was added NaH (2.03 g, 50.7 mmol, 60% purity, 1.5 equiv) at 0° C. under N₂. The mixture was stirred at 0° C. for 1 h. To the mixture was added a solution of 2-bromo-1,1-dimethoxy-ethane (6.29 g, 37.2 mmol, 4.37 mL, 1.1 equiv) in THE (5 mL) dropwise at 0° C. The mixture was heated to 70° C. and stirred for 12 h. The reaction mixture was quenched with sat. aq. NH₄Cl (100 mL) and water (100 mL) and the mixture was extracted with ethyl acetate (100 mL*3). The combined organic phase was washed with brine (100 mL), dried with anhydrous Na₂SO₄, filtered and concentrated in vacuo. The residue was purified by column chromatography on silica gel (Petroleum ether/Ethyl acetate=1/0 to 0/1) to afford 2-[2-[2-[2-[2-[2-(2,2-dimethoxyethoxy)ethoxy]ethoxy]ethoxy]ethoxy]ethoxy]ethoxymethylbenzene (9.40 g, 20.4 mmol, 60% yield) as a yellow oil. ¹H NMR (400 MHz, DMSO-d₆) δ 7.38-7.25 (m, 5H), 4.49 (s, 2H), 4.44 (t, 1H, J=5.2 Hz), 3.56-3.49 (m, 24H), 3.38-3.42 (m, 2H), 3.26 (s, 6H).

### 5. Preparation of Compound 4

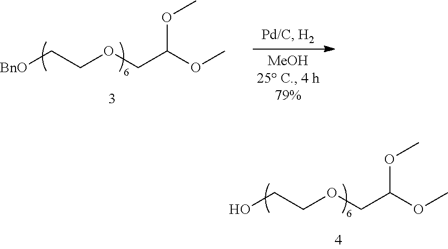

To a solution of 2-[2-[2-[2-[2-[2-(2,2-dimethoxyethoxy)ethoxy]ethoxy]ethoxy]ethoxy]ethoxy]ethoxymethylbenzene (7.9 g, 17.2 mmol, 1.0 equiv) in MeOH (150 mL) was added dry Pd/C (2.0 g, 10% purity) under N₂. The mixture was degassed and stirred at 25° C. for 4 h under H₂ (15 psi). The reaction mixture was filtered and the filtrate was concentrated in vacuo to afford 2-[2-[2-[2-[2-[2-(2,2-dimethoxyethoxy)ethoxy]ethoxy]ethoxy]ethoxy]ethoxy]ethanol (5.00 g, 13.5 mmol, 79% yield) as a colorless oil. ¹H NMR (400 MHz, DMSO-d₆) δ 4.56 (t, 1H, J=5.6 Hz), 4.44 (t, 1H, J=5.2 Hz), 3.55-3.50 (m, 22H), 3.42-3.38 (m, 4H), 3.26 (s, 6H).

### 6. Preparation of Compound 5

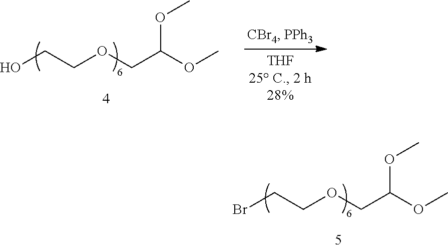

To a mixture of 2-[2-[2-[2-[2-[2-(2,2-dimethoxyethoxy)ethoxy]ethoxy]ethoxy]ethoxy]ethoxy]ethanol (2.0 g, 5.4 mmol, 1.0 equiv) in THF (100 mL) were added PPh₃ (2.7 g, 10.3 mmol, 1.9 equiv) and CBr₄ (3.4 g, 10.3 mmol, 1.9 equiv) at 25° C. Then the mixture was stirred at 25° C. for 2 h. The mixture was filtered and the filtrate was concentrated in vacuo. The residue was diluted with ethyl acetate (10 mL) and to the mixture was added Petroleum ether (100 mL) slowly. The mixture was filtered and the filtrate was concentrated in vacuo. The residue was purified by column chromatography on silica gel (Petroleum ether/Ethyl acetate=1/1 to 0/1) to afford 2-[2-[2-[2-[2-[2-(2-bromoethoxy)ethoxy]ethoxy]ethoxy]ethoxy]ethoxy]-1,1-dimethoxy-ethane (0.65 g, 1.5 mmol, 28% yield) as a colorless oil. ¹H NMR (400 MHz, CDCl₃): δ 4.52 (t, 1H, J=5.2 Hz), 3.82 (t, 2H, J=6.4 Hz), 3.75-3.60 (m, 20H), 3.55 (d, 2H, J=5.2 Hz), 3.48 (t, 2H, J=6.4 Hz), 3.40 (s, 6H).

### 7. Preparation of Compound 6

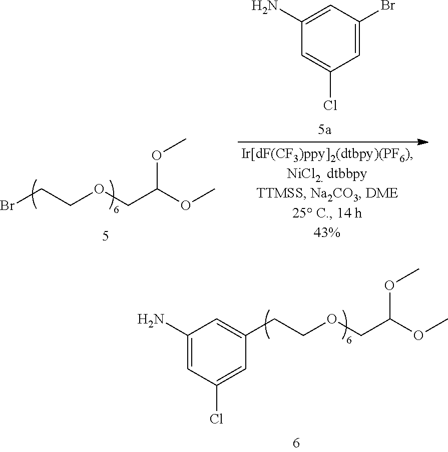

A mixture 3-bromo-5-chloro-aniline (232 mg, 1.13 mmol, 1.0 equiv), 2-[2-[2-[2-[2-[2-(2-bromoethoxy)ethoxy]ethoxy]ethoxy]ethoxy]ethoxy]-1,1-dimethoxy-ethane (537 mg, 1.24 mmol, 1.1 equiv), Ir[dF(CF₃)ppy]₂(dtbpy)(PF₆) (12.6 mg, 0.01 mmol, 0.01 equiv), NiCl₂-dtbbpy (2.24 mg, 0.006 mmol, 0.005 equiv), TTMSS (280 mg, 1.13 mmol, 0.35 mL, 1.0 equiv) and Na₂CO₃ (239 mg, 2.25 mmol, 2.0 equiv) in DME (2 mL) was irradiated with 34 W LED lamp (7 cm away) with cooling fan to keep the reaction temperature at 25° C. and stirred for 14 h under N₂. The mixture was filtered and the filtrate was concentrated in vacuo. The mixture was purified by column chromatography on silica gel (Ethyl acetate/Methanol=10/1) and then prep-HPLC (column: Waters Xbridge C18 150*50 mm*10 μm; mobile phase: [water(10 mM NH₄HCO₃)-ACN]; B %: 26%-56%) to afford 3-chloro-5-[2-[2-[2-[2-[2-[2-(2,2-dimethoxyethoxy)ethoxy]ethoxy]ethoxy]ethoxy]ethoxy]ethyl]aniline (235 mg, 0.45 mmol, 43% yield) as a light yellow oil. ¹H NMR (400 MHz, CDCl₃): δ 6.97-6.64 (m, 3H), 4.50-4.55 (m, 1H), 3.76-3.60 (m, 24H), 3.56 (d, 2H, J=4.8 Hz), 3.41 (s, 6H), 2.80-2.84 (m, 2H). LC-MS: MS (ES⁺): RT=0.744 min, m/z=480.1 [M+H⁺].

### 8. Preparation of Compound 7

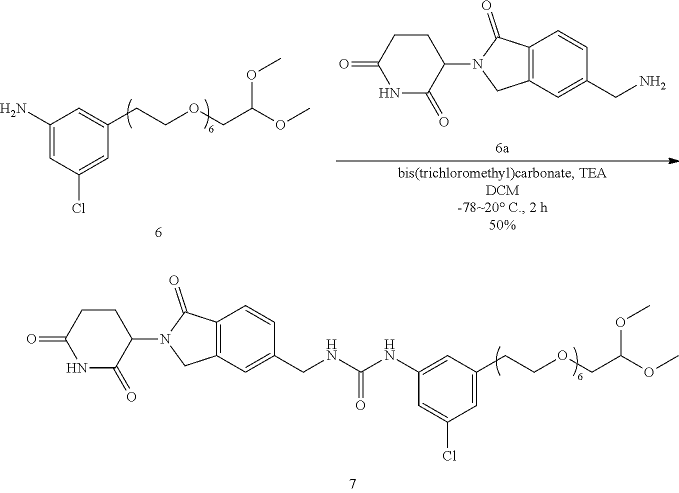

To a mixture of bis(trichloromethyl)carbonate (117 mg, 0.4 mmol, 1.0 equiv) in DCM (18 mL) was added a solution of TEA (400 mg, 4.0 mmol, 0.55 mL, 10 equiv) and 3-chloro-5-[2-[2-[2-[2-[2-[2-(2,2-dimethoxyethoxy)ethoxy]ethoxy]ethoxy]ethoxy]ethoxy]ethyl]aniline (190 mg, 0.4 mmol, 1.0 equiv) in DCM (3 mL) at −78° C. The mixture was stirred for 0.5 h. To the mixture was added 3-[5-(aminomethyl)-1-oxo-isoindolin-2-yl]piperidine-2,6-dione (147 mg, 0.5 mmol, 1.2 equiv, HCl salt) and the mixture was stirred at 20° C. for 12 h. The reaction mixture was poured into sat. aq. NaHCO₃ (10 mL) at 0° C. and the mixture was extracted with DCM (10 mL*2). The combined organic layers were washed with brine (10 mL*2), dried over anhydrous Na₂SO₄, filtered and concentrated in vacuo. The residue was purified by was purified by prep-TLC (Dichloromethane/Methanol=10/1) to afford 1-[3-chloro-5-[2-[2-[2-[2-[2-[2-(2,2-dimethoxyethoxy)ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (55.0 mg, 0.7 mmol, 18% yield) as a light yellow oil. LC-MS: MS (ES⁺): RT=0.880 min, m/z=796.3 [M+H⁺+17].

### 9. Preparation of Compound 8

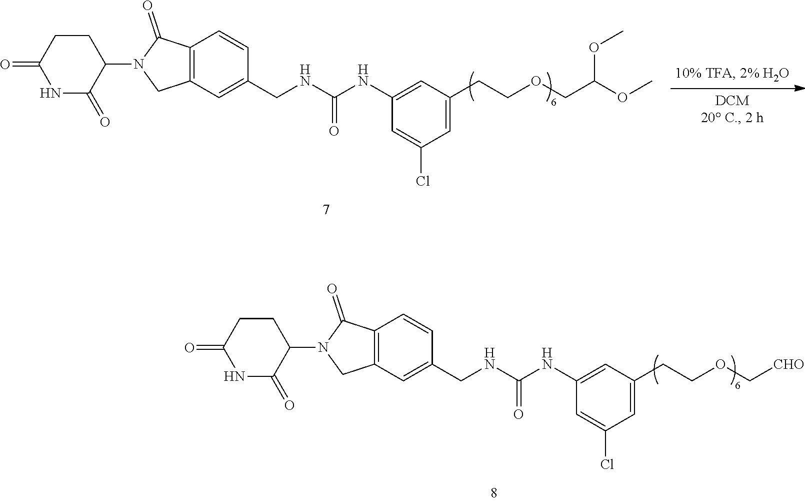

A mixture of 1-[3-chloro-5-[2-[2-[2-[2-[2-[2-(2,2-dimethoxyethoxy)ethoxy]ethoxy]-ethoxy]ethoxy]-ethoxy]ethyl]phenyl]-3-[[2-(2,6-dioxo-3-piperidyl)-1-oxo-isoindolin-5-yl]methyl]urea (35 mg, 0.045 mmol, 1 equiv) in DCM (3 mL) and H₂O (0.06 mL) was added TFA (0.3 mL) was stirred at 20° C. for 2 h. The pH of the reaction mixture was adjusted to 7˜8 by addition of Et₃N at 0° C. to afford a DCM solution 1-[3-chloro-5-[2-[2-[2-[2-[2-[2-(2-oxoethoxy)ethoxy]ethoxy]ethoxy]ethoxy]ethoxy]ethyl]-phenyl]-3-[[2-(2,6-dioxo-3-piperidyl)-1-oxo-isoindolin-5-yl]methyl]urea (33.0 mg, crude). LC-MS: MS (ES⁺): RT=0.726 min, m/z=733.3 [M+H⁺].

### 10. Preparation of I-23

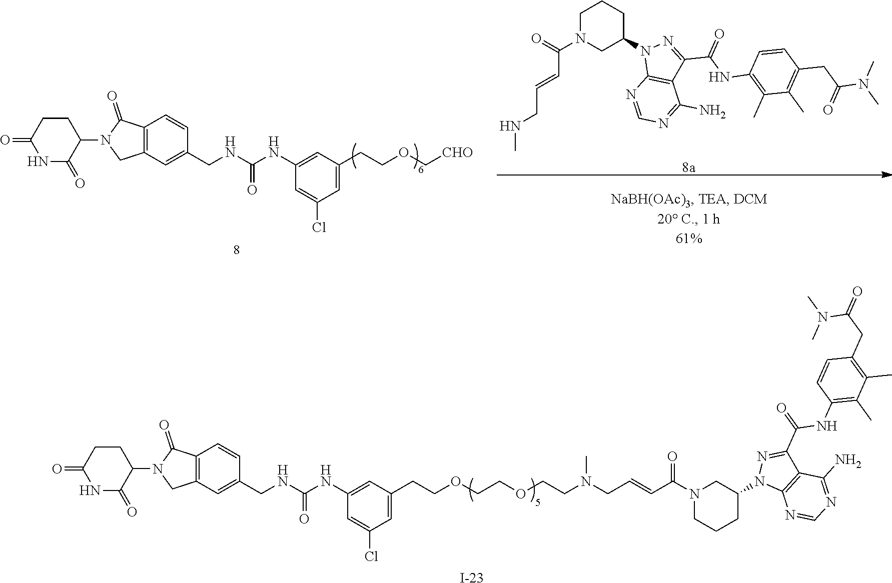

To a mixture of 1-[3-chloro-5-[2-[2-[2-[2-[2-[2-(2-oxoethoxy)ethoxy]ethoxy]ethoxy]ethoxy]ethoxy]ethyl]phenyl]-3-[[2-(2,6-dioxo-3-piperidyl)-1-oxo-isoindolin-5-yl]methyl]urea (33.0 mg, 0.045 mmol, 1.06 equiv) in DCM (1 mL) were added TEA (13 mg, 0.13 mmol, 3.0 equiv), (1S)-4-amino-N-[4-[2-(dimethylamino)-2-oxo-ethyl]-2,3-dimethyl-phenyl]-1-[1-[(E)-4-(methylamino)but-2-enoyl]-3-piperidyl]pyrazolo[3,4-d]pyrimidine-3-carboxamide (28 mg, 0.042 mmol, 1.0 equiv, TFA salt) and NaBH(OAc)₃ (90 mg, 0.42 mmol, 10 equiv) at 20° C. The reaction mixture was stirred at 20° C. for 1 h. The reaction mixture was diluted with water (5 mL) and the mixture was extracted with DCM (5 mL*2). The combined organic phase was washed with brine (5 mL*2), dried with anhydrous Na₂SO₄, filtered and concentrated in vacuo. The residue was purified by prep-HPLC (column: Phenomenex Gemini-NX C18 75*30 mm*3 μm; mobile phase: [water(0.225% FA)-ACN]; B %: 18%-48%, 5 min) to afford 4-amino-1-[(3R)-1-[(E)-4-[2-[2-[2-[2-[2-[2-[2-[3-chloro-5-[[2-(2,6-dioxo-3-piperidyl)-1-oxo-isoindolin-5-yl]methylcarbamoylamino]phenyl]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo [3,4-d]pyrimidine-3-carboxamide (33 mg, 0.026 mmol, 61% yield, 100% purity) as a yellow solid. ¹H NMR (400 MHz, DMSO-d₆): δ 10.98 (s, 1H), 10.24-10.01 (m, 1H), 8.96 (s, 1H), 8.62-8.57 (m, 1H), 8.26 (s, 1H), 8.10-8.06 (m, 1H), 7.71-7.67 (m, 1H), 7.55-7.49 (m, 2H), 7.46-7.42 (m, 1H), 7.20-7.12 (m, 1H), 7.10-7.06 (m, 1H), 7.02-6.94 (m, 2H), 6.86-6.82 (m, 1H), 6.71-6.48 (m, 2H), 5.16-5.04 (m, 1H), 4.81-4.57 (m, 1H), 4.47-4.39 (m, 3H), 4.34-4.28 (m, 1H), 4.22-4.04 (m, 1H), 3.72 (s, 2H), 3.60-3.55 (s, 4H), 3.55-3.45 (m, 20H), 3.43-3.39 (m, 3H), 3.17-3.12 (m, 1H), 3.07-3.01 (m, 4H), 2.89-2.85 (m, 4H), 2.75-2.70 (m, 2H), 2.63-2.58 (m, 2H), 2.42-2.37 (m, 2H), 2.20-2.14 (m, 6H), 2.13-2.08 (m, 5H), 2.03-1.96 (m, 2H), 1.64-1.55 (m, 1H). LC-MS: MS (ES⁺): RT=2.695 min, m/z=633.0 [1/2M+H⁺]; LCMS method: LC-MS METHOD 10.

### Example 24—Preparation of Compound I-24

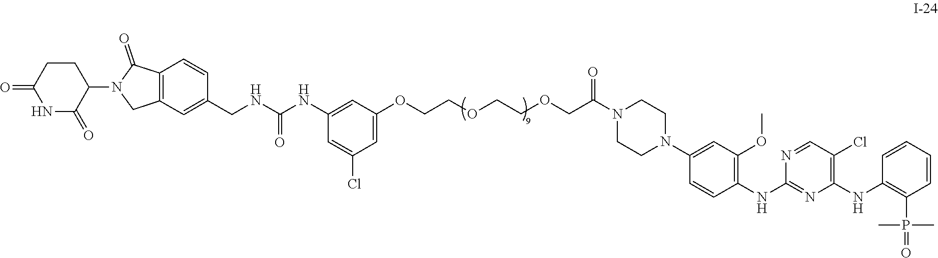

The title compound was prepared according to the following procedures.

### 1. Preparation of Compound 2

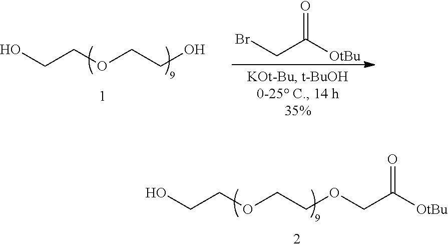

To a solution of 2-[2-[2-[2-[2-[2-[2-[2-[2-(2-hydroxyethoxy)ethoxy]ethoxy]ethoxy]ethoxy]ethoxy]ethoxy]ethoxy]ethoxy]ethanol (10.0 g, 21.8 mmol, 1.0 equiv) in t-BuOH (100 mL) was added t-BuOK (2.32 g, 20.7 mmol, 0.95 equiv) at 0° C. The mixture was stirred at 25° C. for 2 h. To the mixture was added tert-butyl 2-bromoacetate (4.72 g, 24.2 mmol, 3.58 mL, 1.1 equiv) and the mixture was stirred at 25° C. for 12 h. The reaction mixture was diluted with water (100 mL) and the mixture was extracted with ethyl acetate (50 mL*3). The combined organic phase was washed with brine (50 mL), dried with anhydrous Na₂SO₄, filtered and concentrated in vacuo. The residue was purified by column chromatography on silica gel (Dichloromethane/Methanol=10/1) to afford tert-butyl 2-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]acetate (4.34 g, 7.58 mmol, 35% yield) as a yellow oil. ¹HNMR (400 MHz, CDCl₃): δ 3.99 (s, 2H), 3.71-3.65 (m, 9H), 3.64-3.61 (m, 30H), 3.59-3.57 (m, 2H), 1.45 (s, 9H).

### 2. Preparation of Compound 3

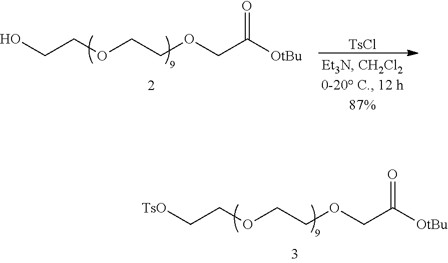

To a solution of tert-butyl 2-[2-[2-[2-[2-[2-[2-[2-[2-[2-(2-hydroxyethoxy)ethoxy]ethoxy]ethoxy]-ethoxy]ethoxy]ethoxy]ethoxy]ethoxy]ethoxy]acetate (1.60 g, 2.79 mmol, 1.0 equiv) in CH₂Cl₂ (16 mL) were added Et₃N (848 mg, 8.38 mmol, 1.17 mL, 3.0 equiv) and tosylchloride (1.07 g, 5.59 mmol, 2.0 equiv) at 0° C. The reaction mixture was stirred at 20° C. for 12 h. The reaction mixture was concentrated in vacuo. The residue was purified by column chromatography on silica gel (Dichloromethane/Methanol=10/1) to afford tert-butyl 2-[2-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethoxy]ethoxy]acetate (1.77 g, 2.44 mmol, 87% yield) as a yellow oil. ¹HNMR (400 MHz, CDCl₃): δ 7.86-7.73 (m, 2H), 7.38-7.30 (m, 2H), 4.18-4.14 (m, 2H), 4.02 (s, 2H), 3.71-3.67 (m, 6H), 3.66-3.62 (m, 28H), 3.59-3.56 (m, 4H), 2.45 (s, 3H), 1.47 (s, 9H).

### 3. Preparation of Compound 4

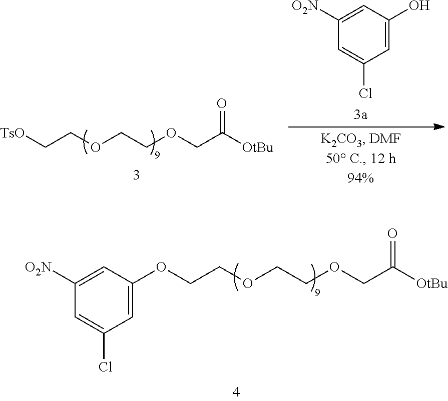

To a mixture of tert-butyl 2-[2-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethoxy]ethoxy]acetate (1.60 g, 2.20 mmol, 1.1 equiv) and 3-chloro-5-nitro-phenol (350 mg, 2.02 mmol, 1.0 equiv) in DMF (16 mL) was added K₂CO₃ (557 mg, 4.03 mmol, 2.0 equiv) at 20° C. The reaction mixture was stirred at 50° C. for 12 h. The reaction mixture was diluted with water (15 mL) and the mixture was extracted with ethyl acetate (15 mL*3). The combined organic phase was washed with brine (15 mL), dried with anhydrous Na₂SO₄, filtered and concentrated in vacuo. The residue was purified by column chromatography on silica gel (Dichloromethane/Methanol=20/1) to afford tert-butyl 2-[2-[2-[2-[2-[2-[2-[2-[2-[2-[2-(3-chloro-5-nitro-phenoxy)ethoxy]ethoxy]ethoxy]ethoxy]ethoxy]ethoxy]ethoxy]-ethoxy]ethoxy]ethoxy]acetate (1.38 g, 1.90 mmol, 94% yield) as a yellow oil. ¹H NMR (400 MHz, DMSO-d₆): δ 6.20-6.16 (m, 1H), 6.12-6.08 (m, 1H), 6.0-6.03 (m, 1H), 5.59-5.15 (m, 2H), 3.99-3.96 (m, 4H), 3.70-3.67 (m, 2H), 3.58-3.54 (m, 6H), 3.51-3.49 (m, 30H), 1.42 (s, 9H).

### 4. Preparation of Compound 5

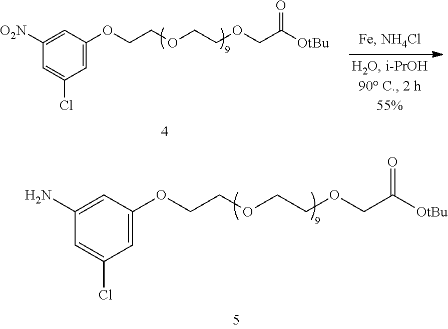

To a solution of tert-butyl 2-[2-[2-[2-[2-[2-[2-[2-[2-[2-[2-(3-chloro-5-nitro-phenoxy)ethoxy]-ethoxy]ethoxy]ethoxy]ethoxy]ethoxy]ethoxy]ethoxy]ethoxy]ethoxy]acetate (1.28 g, 1.76 mmol, 1.0 equiv) in i-PrOH (12 mL) and H₂O (3 mL) were added NH₄Cl (940 mg, 17.6 mmol, 10.0 equiv) and Fe (785 mg, 14.1 mmol, 8.0 equiv). The reaction mixture was stirred at 90° C. for 2 h. The reaction mixture was diluted with water (20 mL) and the mixture was extracted with ethyl acetate (15 mL*3). The combined organic phase was washed with brine (10 mL), dried with anhydrous Na₂SO₄, filtered and concentrated in vacuo. The residue was purified by prep-HPLC (column: Phenomenex luna C18 150*40 mm*15 μm; mobile phase: [water(0.225% FA)-ACN]; B %: 37%-67%, 10 min) to afford tert-butyl 2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]ethoxy]ethoxy]acetate (670 mg, 0.96 mmol, 55% yield) as a yellow oil. ¹H NMR (400 MHz, CDCl₃): δ 8.57-8.47 (m, 1H), 8.01 (s, 1H), 7.73 (d, 1H, J=7.6 Hz), 7.68 (s, 1H), 7.48 (s, 1H), 7.39 (d, 1H, J=7.6 Hz), 7.19-7.16 (m, 2H), 7.09 (s, 1H), 6.75 (s, 1H), 5.19-5.12 (m, 1H), 4.51-4.46 (m, 2H), 4.36-4.24 (m, 2H), 4.00 (s, 2H), 3.64-3.55 (m, 30H), 2.98-2.79 (m, 5H), 2.75-2.70 (m, 2H), 2.40-2.30 (m, 1H), 2.23-2.16 (m, 1H), 1.47 (s, 9H).

### 5. Preparation of Compound 6

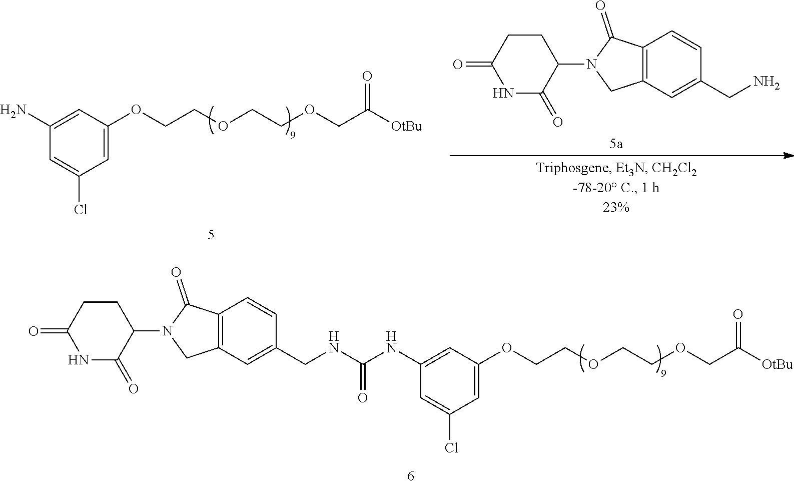

To a solution of bis(trichloromethyl)carbonate (42.5 mg, 0.14 mmol, 0.5 equiv) in CH₂Cl₂ (3 mL) were added Et₃N (145 mg, 1.43 mmol, 0.2 mL, 5.0 equiv) and a solution of tert-butyl 2-[2-[2-[2-[2-[2-[2-[2-[2-[2-[2-(3-amino-5-chloro-phenoxy)ethoxy]ethoxy]ethoxy]ethoxy]ethoxy]ethoxy]ethoxy]ethoxy]-ethoxy]ethoxy]acetate (200 mg, 0.29 mmol, 1.0 equiv) in CH₂Cl₂ (2 mL) at −78° C. The mixture was stirred −78° C. for 0.5 h. To the mixture was added 3-[5-(aminomethyl)-1-oxo-isoindolin-2-yl]piperidine-2,6-dione (88.7 mg, 0.29 mmol, 1.0 equiv, HCl salt) at −78° C. The mixture was stirred at 20° C. for 0.5 h. The reaction mixture was diluted with sat. aq. NaHCO₃ (10 mL) and the mixture was extracted with ethyl acetate (5 mL*3). The combined organic phase was washed with brine (10 mL), dried with anhydrous Na₂SO₄, filtered and concentrated in vacuo. The residue was purified by prep-TLC on silica gel (CH₂Cl₂/MeOH=10/1) to tert-butyl 2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]acetate (65.0 mg, 65.2 μmol, 23% yield) as a yellow oil. ¹H NMR (400 MHz, DMSO-d₆): δ 10.97 (s, 1H), 8.86 (s, 1H), 7.72-7.66 (m, 1H), 7.51 (s, 1H), 7.46-7.41 (m, 1H), 7.18-7.13 (m, 1H), 6.97-6.93 (m, 1H), 6.87 (t, 1H, J=6.0 Hz), 6.56 (s, 1H), 5.76 (s, 1H), 5.15-5.06 (m, 1H), 4.47-4.39 (m, 3H), 4.35-4.27 (m, 1H), 4.06-4.03 (m, 2H), 3.97 (s, 2H), 3.73-3.69 (m, 2H), 3.57-3.47 (m, 39H), 1.41 (s, 9H).

### 6. Preparation of Compound 7

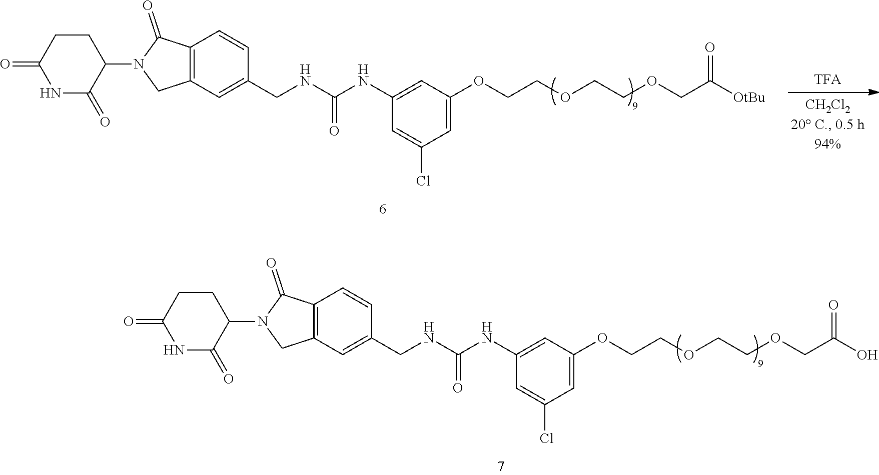

A mixture of tert-butyl 2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]acetate (65.0 mg, 65.2 μmol, 1.0 equiv) in CH₂Cl₂ (1 mL) and TFA (0.5 mL) at 20° C. The reaction mixture was stirred at 20° C. for 0.5 h. The reaction mixture was concentrated in vacuo to afford 2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]acetic acid (61.0 mg, 61.6 μmol, 94% yield) as a yellow oil. LC-MS: MS (ES⁺): RT=0.773 min, m/z=941.0 [M+H⁺].

### 7. Preparation of I-24

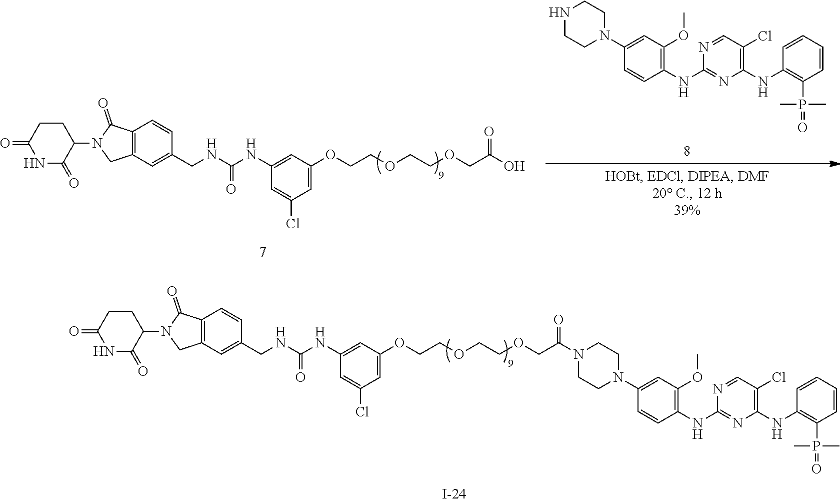

To a mixture of 2-[2-[2-[2-[2-[2-[2-[2-[2-[2-[2-[3-chloro-5-[[2-(2,6-dioxo-3-piperidyl)-1-oxo-isoindolin-5-yl]methylcarbamoylamino]phenoxy]ethoxy]ethoxy]ethoxy]ethoxy]ethoxy]ethoxy]ethoxy]ethoxy]ethoxy]ethoxy]acetic acid (61.0 mg, 64.8 μmol, 1.0 equiv) and 5-chloro-N4-(2-dimethylphosphorylphenyl)-N2-(2-methoxy-4-piperazin-1-yl-phenyl)pyrimidine-2,4-diamine (47.0 mg, 78.2 μmol, 1.2 equiv, TFA salt) in DMF (0.7 mL) were added HOBt (13.1 mg, 97.2 μmol, 1.5 equiv), DIPEA (67.0 mg, 0.51 mmol, 90.3 μL, 8.0 equiv) and EDCI (18.6 mg, 97.2 μmol, 1.5 equiv) at 20° C. The reaction mixture was stirred at 20° C. for 12 h. The reaction mixture was concentrated in vacuo. The residue was purified by prep-HPLC (column: Unisil 3-100 C18 Ultra 150*50 mm*3 μm; mobile phase: [water(0.225% FA)-ACN]; B %: 30%-60%, 10 min) to afford 1-[3-chloro-5-[2-[2-[2-[2-[2-[2-[2-[2-[2-[2-[2-[4-[4-[[5-chloro-4-(2-dimethyl phosphorylanilino)pyrimidin-2-yl]amino]-3-methoxy-phenyl]piperazin-1-yl]-2-oxo-ethoxy]ethoxy]ethoxy]ethoxy]ethoxy]ethoxy]ethoxy]ethoxy]ethoxy]ethoxy]ethoxy]phenyl]-3-[[2-(2,6-dioxo-3-piperidyl)-1-oxo-isoindolin-5-yl]methyl]urea (37.5 mg, 25.2 μmol, 39% yield, 95% purity) as a white solid. ¹H NMR (400 MHz, DMSO-d₆): δ 11.18 (s, 1H), 10.97 (brs, 1H), 8.87 (s, 1H), 8.55-8.40 (m, 1H), 8.07 (s, 2H), 7.71-7.66 (m, 1H), 7.56-7.50 (m, 2H), 7.44 (t, 2H, J=7.6 Hz), 7.38-7.33 (m, 1H), 7.16 (brs, 1H), 7.13-7.08 (m, 1H), 6.95 (brs, 1H), 6.90 (brs, 1H), 6.68 (brs, 1H), 6.56 (brs, 1H), 6.51-6.48 (m, 1H), 5.15-5.06 (m, 1H), 4.46-4.29 (m, 5H), 4.22-4.19 (m, 2H), 4.06-4.03 (m, 2H), 3.77 (s, 3H), 3.72-7.69 (m, 2H), 3.58-3.55 (m, 7H), 3.51-3.46 (m, 29H), 3.16-3.11 (m, 4H), 2.68-2.59 (m, 3H), 2.41-2.31 (m, 2H), 2.02-1.97 (m, 1H), 1.78-1.74 (m, 7H). LC-MS: MS (ES⁺): RT=2.435 min, m/z=1411.0 [M+H⁺]; LCMS method: LC-MS METHOD 10

### Example 25—Preparation of Additional Heterobifunctional Compounds

The compounds in Table 2 below were prepared based on procedures described herein above. Characterization data for compounds in Table 2 is provided in Table 2A.

TABLE 2

| Compd | |
|---|---|
| No. | Structure |
| I-25 | 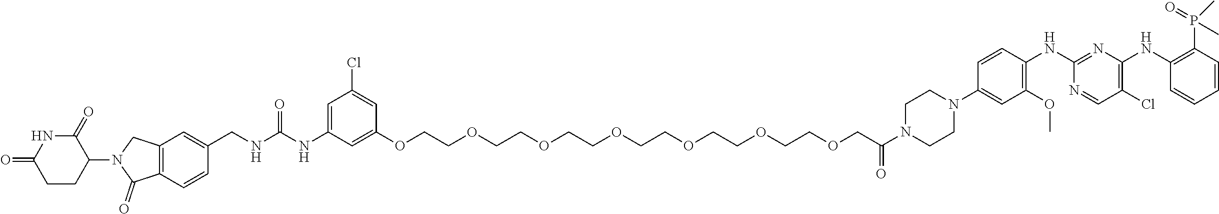 [SMILES: COc1cc(N2CCN(C(=O)COCCOCCOCCOCCOCCOCCOc3cc(Cl)cc(NC(=O)NCc4ccc5c(c4)CN(C4CCC(=O)NC4=O)C5=O)c3)CC2)ccc1Nc1ncc(Cl)c(Nc2ccccc2P(C)(C)=O)n1] |
| I-26 | 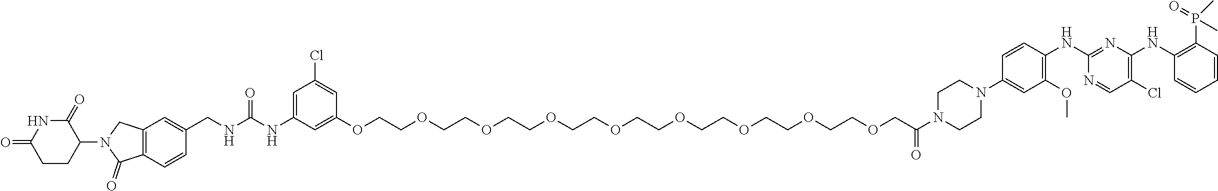 [SMILES: COc1cc(N2CCN(C(=O)COCCOCCOCCOCCOCCOCCOCCOCCOc3cc(Cl)cc(NC(=O)NCc4ccc5c(c4)CN(C4CCC(=O)NC4=O)C5=O)c3)CC2)ccc1Nc1ncc(Cl)c(Nc2ccccc2P(C)(C)=O)n1] |
| I-27 | 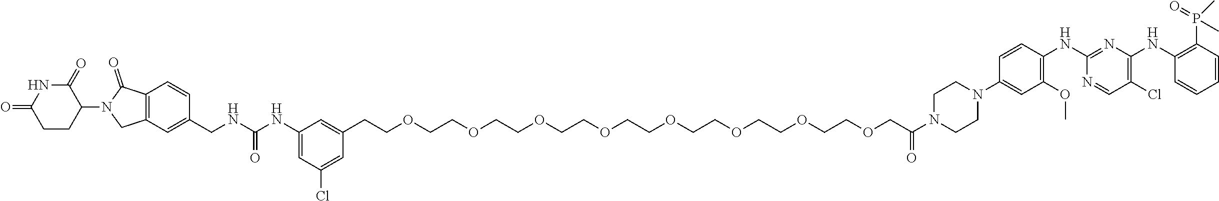 [SMILES: COc1cc(N2CCN(C(=O)COCCOCCOCCOCCOCCOCCOCCOCCc3cc(Cl)cc(NC(=O)NCc4ccc5c(c4)CN(C4CCC(=O)NC4=O)C5=O)c3)CC2)ccc1Nc1ncc(Cl)c(Nc2ccccc2P(C)(C)=O)n1] |
| I-28 | 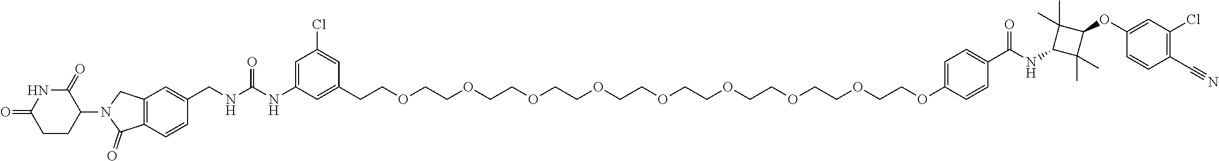 [SMILES: CC1(C)[C@H](NC(=O)c2ccc(OCCOCCOCCOCCOCCOCCOCCOCCOCCc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1] |
| I-29 | 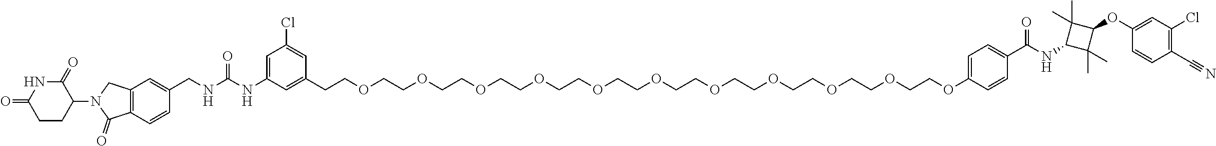 [SMILES: CC1(C)[C@H](NC(=O)c2ccc(OCCOCCOCCOCCOCCOCCOCCOCCOCCOCCOCCc3cc(Cl)cc(NC(=O)NCc4ccc5c(c4)CN(C4CCC(=O)NC4=O)C5=O)c3)cc2)C(C)(C)[C@H]1Oc1ccc(C#N)c(Cl)c1] |
| I-30 | 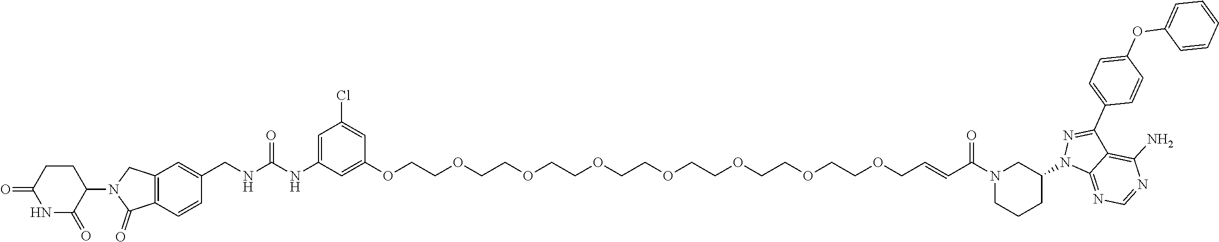 [SMILES: Nc1ncnc2c1c(-c1ccc(Oc3ccccc3)cc1)nn2[C@@H]1CCCN(C(=O)/C=C/COCCOCCOCCOCCOCCOCCOCCOc2cc(Cl)cc(NC(=O)NCc3ccc4c(c3)CN(C3CCC(=O)NC3=O)C4=O)c2)C1] |
| I-31 | 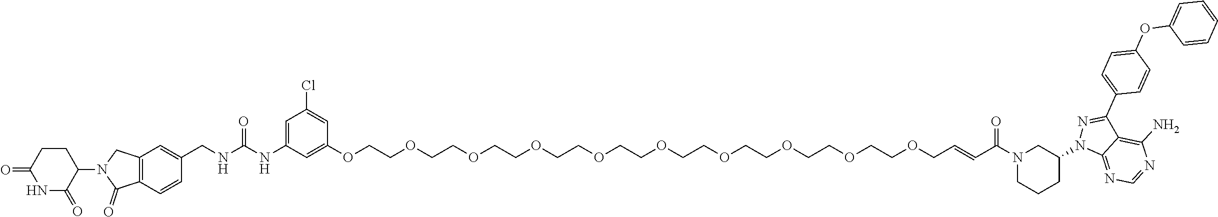 [SMILES: Nc1ncnc2c1c(-c1ccc(Oc3ccccc3)cc1)nn2[C@@H]1CCCN(C(=O)/C=C/COCCOCCOCCOCCOCCOCCOCCOCCOCCOc2cc(Cl)cc(NC(=O)NCc3ccc4c(c3)CN(C3CCC(=O)NC3=O)C4=O)c2)C1] |
| I-32 | 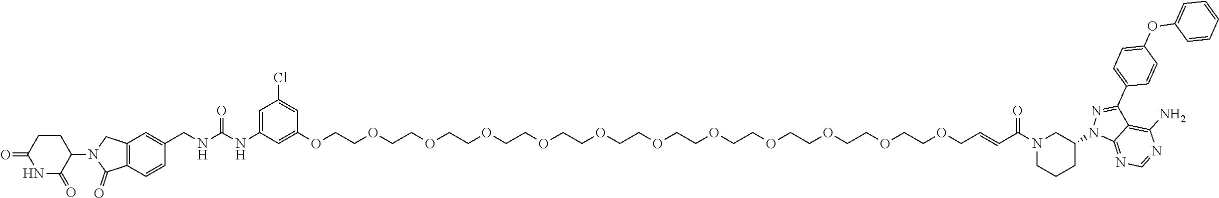 [SMILES: Nc1ncnc2c1c(-c1ccc(Oc3ccccc3)cc1)nn2[C@@H]1CCCN(C(=O)/C=C/COCCOCCOCCOCCOCCOCCOCCOCCOCCOCCOCCOc2cc(Cl)cc(NC(=O)NCc3ccc4c(c3)CN(C3CCC(=O)NC3=O)C4=O)c2)C1] |
| I-33 | 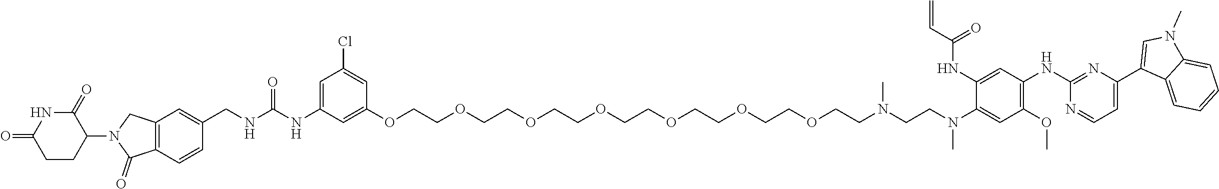 [SMILES: C=CC(=O)Nc1cc(Nc2nccc(-c3cn(C)c4ccccc34)n2)c(OC)cc1N(C)CCN(C)CCOCCOCCOCCOCCOCCOCCOc1cc(Cl)cc(NC(=O)NCc2ccc3c(c2)CN(C2CCC(=O)NC2=O)C3=O)c1] |
| I-34 | 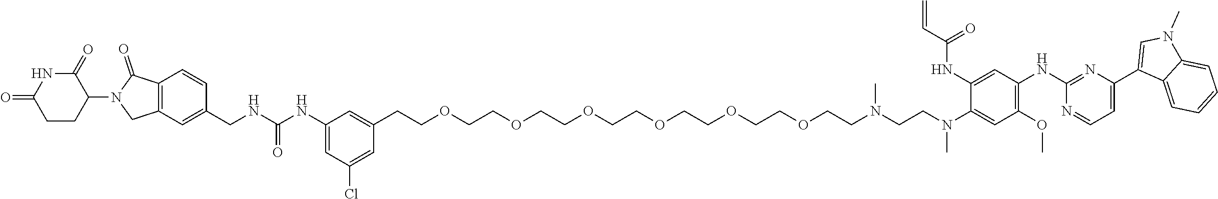 [SMILES: C=CC(=O)Nc1cc(Nc2nccc(-c3cn(C)c4ccccc34)n2)c(OC)cc1N(C)CCN(C)CCOCCOCCOCCOCCOCCOCCc1cc(Cl)cc(NC(=O)NCc2ccc3c(c2)CN(C2CCC(=O)NC2=O)C3=O)c1] |
| I-35 | 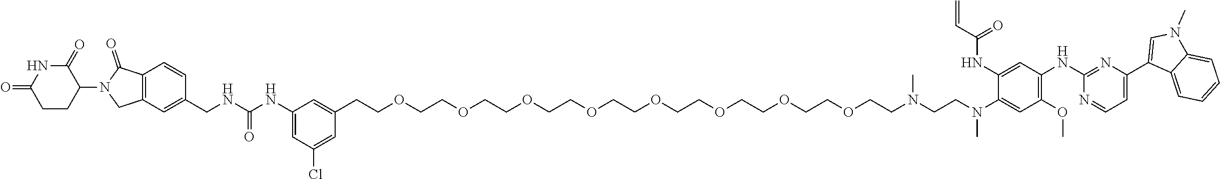 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCc1cc(Cl)cc(NC(=O)NCc2ccc3c(c2)CN(C2CCC(=O)NC2=O)C3=O)c1] |
| I-36 | 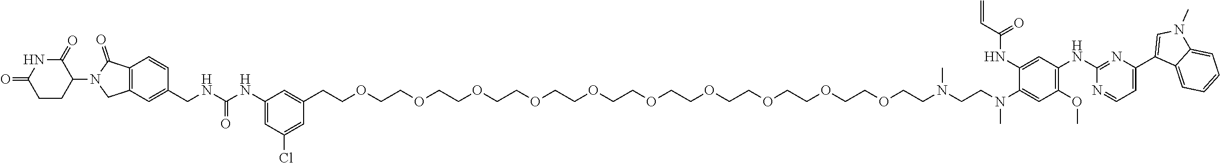 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCOCCOCCc1cc(Cl)cc(NC(=O)NCc2ccc3c(c2)CN(C2CCC(=O)NC2=O)C3=O)c1] |
| I-37 | 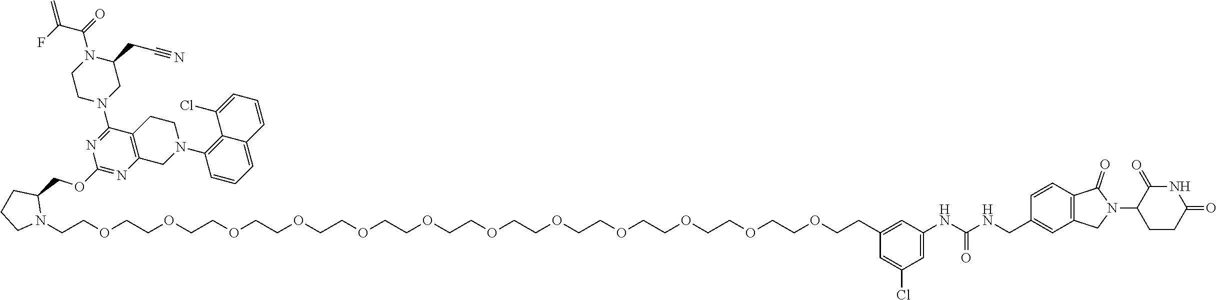 [SMILES: C=C(F)C(=O)N1CCN(c2nc(OC[C@@H]3CCCN3CCOCCOCCOCCOCCOCCOCCOCCOCCOCCOCCOCCOCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-38 | 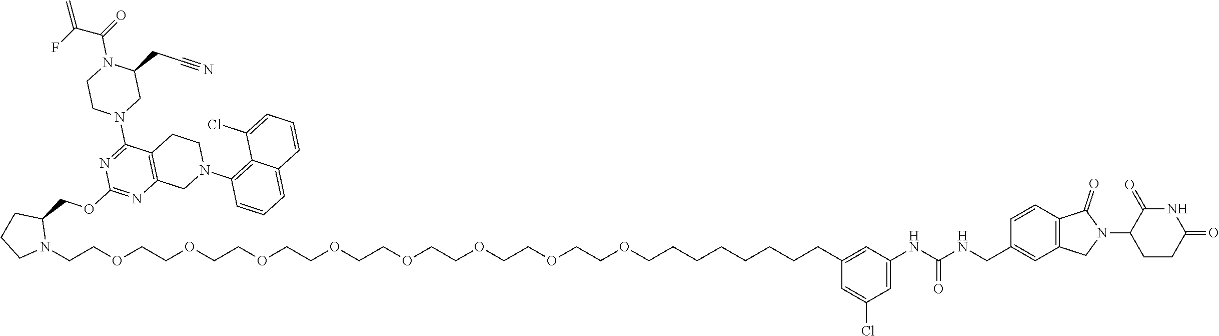 [SMILES: C=C(F)C(=O)N1CCN(c2nc(OC[C@@H]3CCCN3CCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-39 | 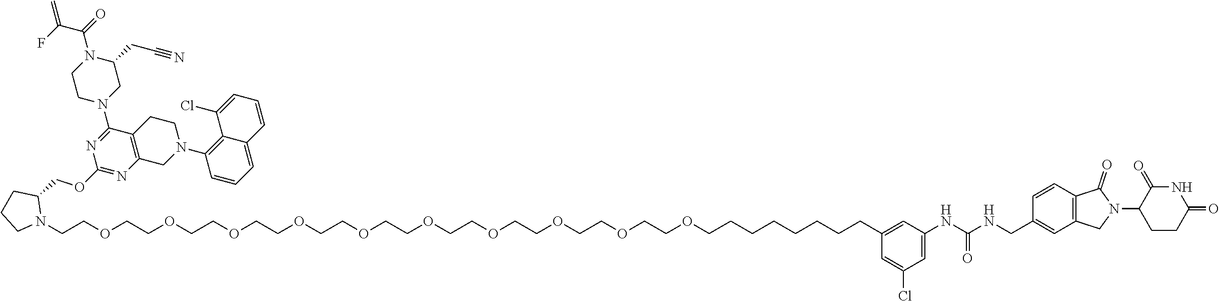 [SMILES: C=C(F)C(=O)N1CCN(c2nc(OC[C@H]3CCCN3CCOCCOCCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@H]1CC#N] |
| I-40 | 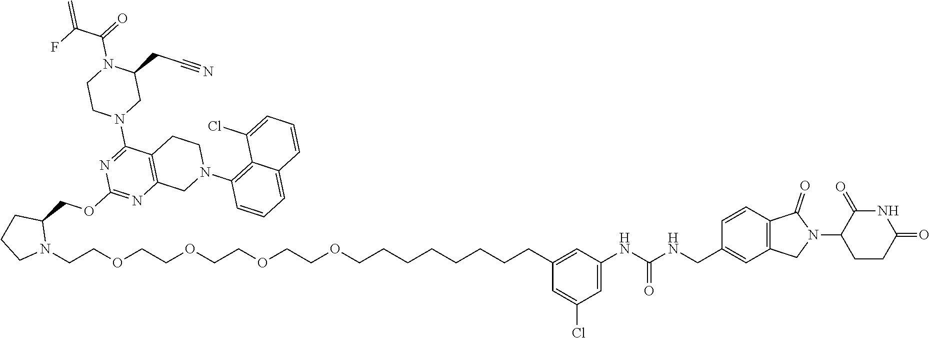 [SMILES: C=C(F)C(=O)N1CCN(c2nc(OC[C@@H]3CCCN3CCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-41 | 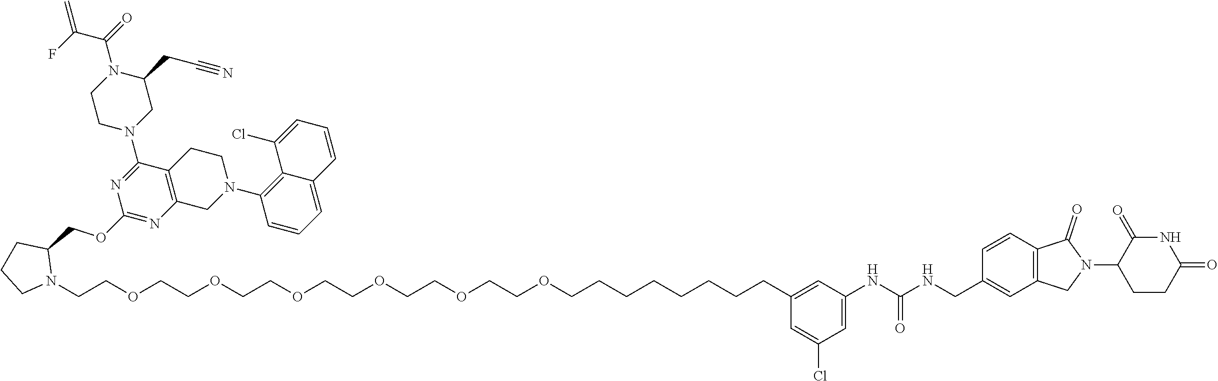 [SMILES: C=C(F)C(=O)N1CCN(c2nc(OC[C@@H]3CCCN3CCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-42 | 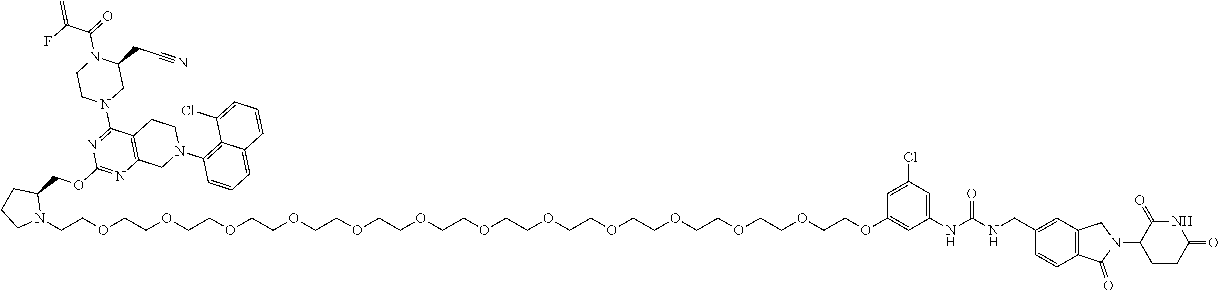 [SMILES: C=C(F)C(=O)N1CCN(c2nc(OC[C@@H]3CCCN3CCOCCOCCOCCOCCOCCOCCOCCOCCOCCOCCOCCOCCOc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-43 | 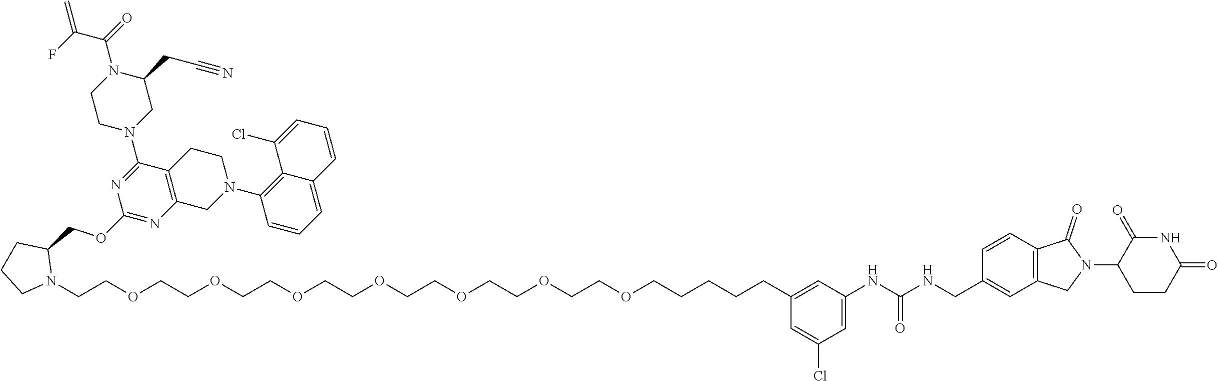 [SMILES: C=C(F)C(=O)N1CCN(c2nc(OC[C@@H]3CCCN3CCOCCOCCOCCOCCOCCOCCOCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-44 | 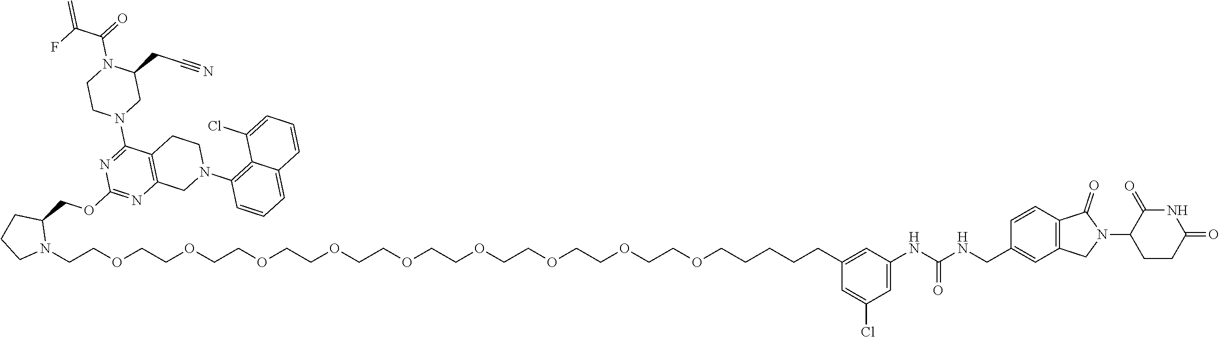 [SMILES: C=C(F)C(=O)N1CCN(c2nc(OC[C@@H]3CCCN3CCOCCOCCOCCOCCOCCOCCOCCOCCOCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-45 | 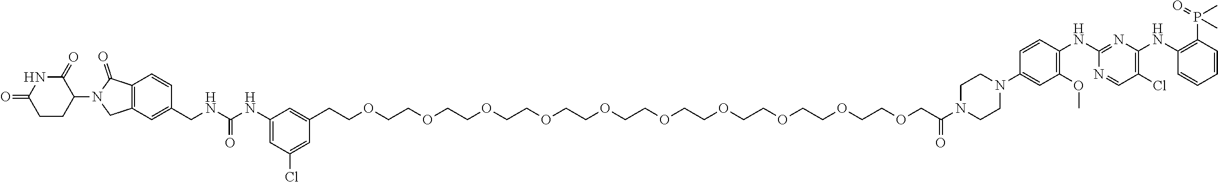 [SMILES: COc1cc(N2CCN(C(=O)COCCOCCOCCOCCOCCOCCOCCOCCOCCOCCc3cc(Cl)cc(NC(=O)NCc4ccc5c(c4)CN(C4CCC(=O)NC4=O)C5=O)c3)CC2)ccc1Nc1ncc(Cl)c(Nc2ccccc2P(C)(C)=O)n1] |
| I-46 | 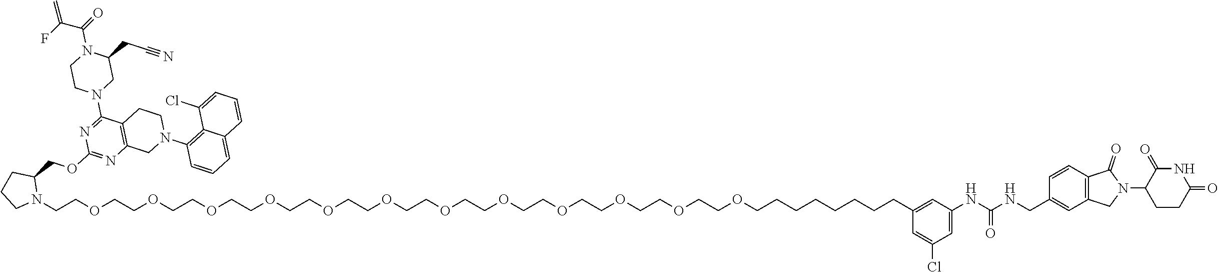 [SMILES: C=C(F)C(=O)N1CCN(c2nc(OC[C@@H]3CCCN3CCOCCOCCOCCOCCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-47 | 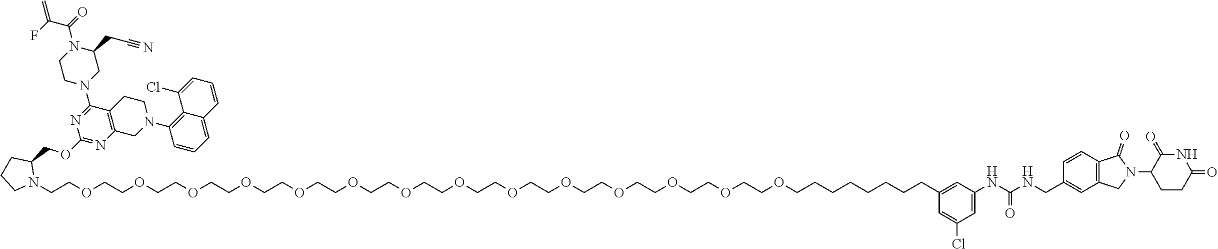 [SMILES: C=C(F)C(=O)N1CCN(c2nc(OC[C@@H]3CCCN3CCOCCOCCOCCOCCOCCOCCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-48 | 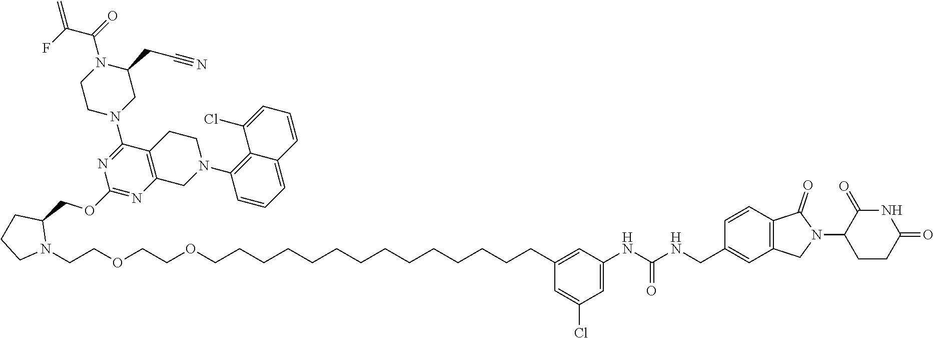 [SMILES: C=C(F)C(=O)N1CCN(c2nc(OC[C@@H]3CCCN3CCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-49 | 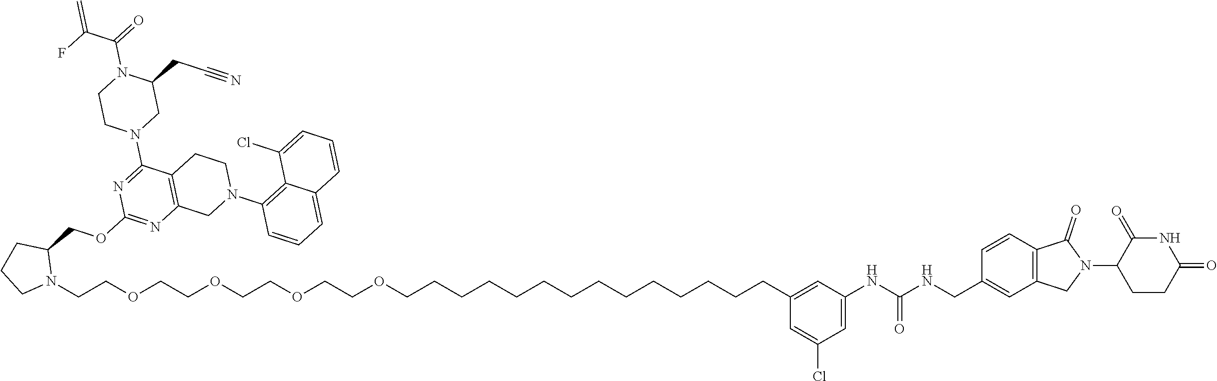 [SMILES: C=C(F)C(=O)N1CCN(c2nc(OC[C@@H]3CCCN3CCOCCOCCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-50 | 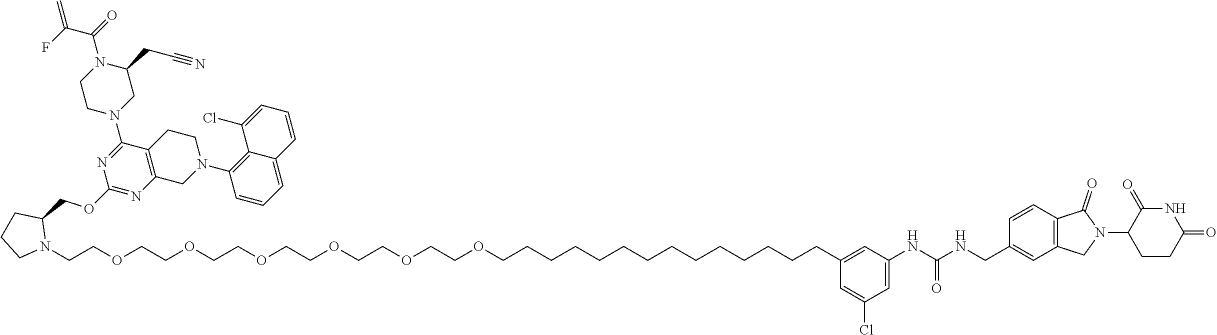 [SMILES: C=C(F)C(=O)N1CCN(c2nc(OC[C@@H]3CCCN3CCOCCOCCOCCOCCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-51 | 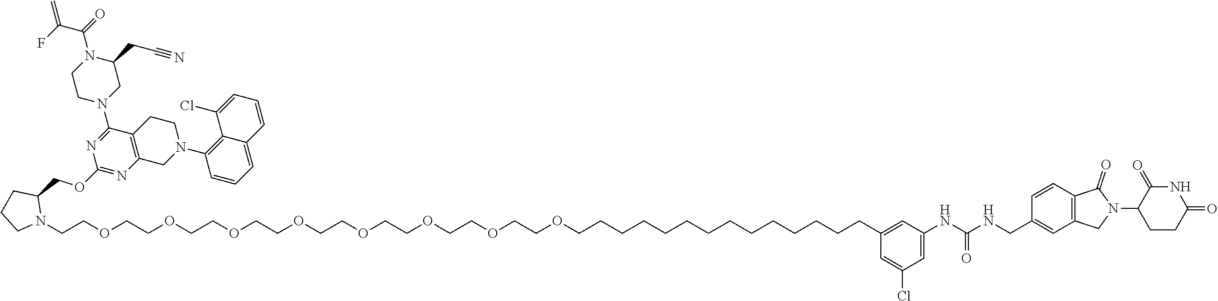 [SMILES: C=C(F)C(=O)N1CCN(c2nc(OC[C@@H]3CCCN3CCOCCOCCOCCOCCOCCOCCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-52 | 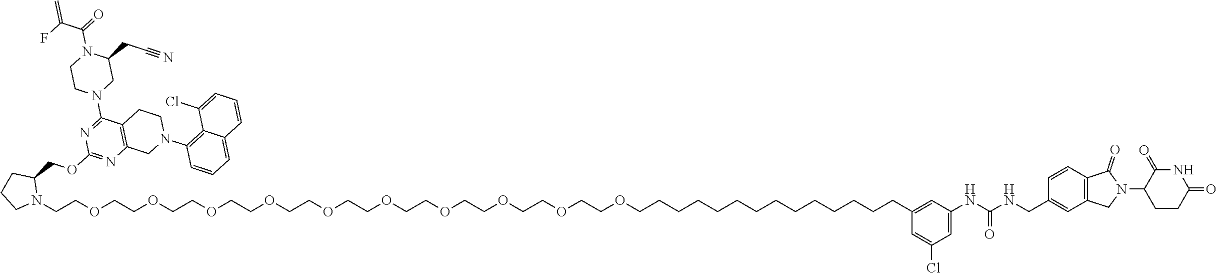 [SMILES: C=C(F)C(=O)N1CCN(c2nc(OC[C@@H]3CCCN3CCOCCOCCOCCOCCOCCOCCOCCOCCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-53 | 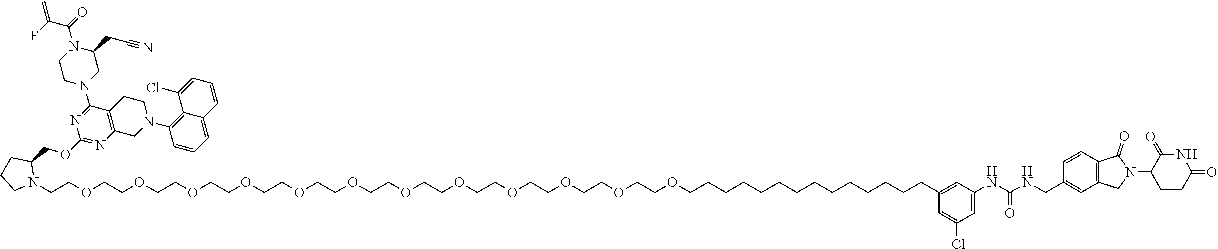 [SMILES: C=C(F)C(=O)N1CCN(c2nc(OC[C@@H]3CCCN3CCOCCOCCOCCOCCOCCOCCOCCOCCOCCOCCOCCOCCCCCCCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-54 | 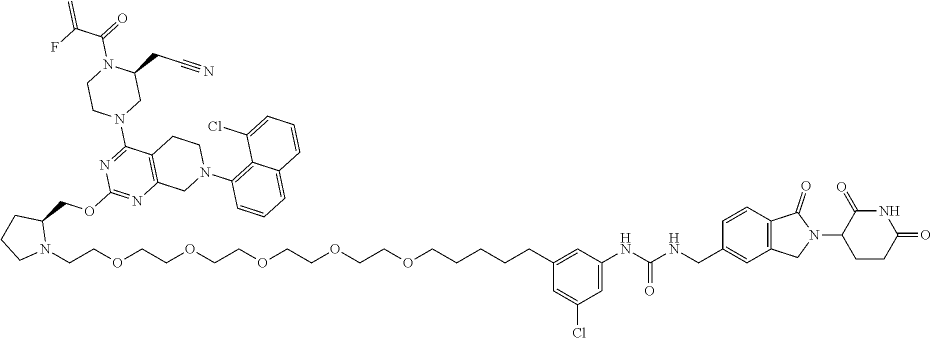 [SMILES: C=C(F)C(=O)N1CCN(c2nc(OC[C@@H]3CCCN3CCOCCOCCOCCOCCOCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-55 | 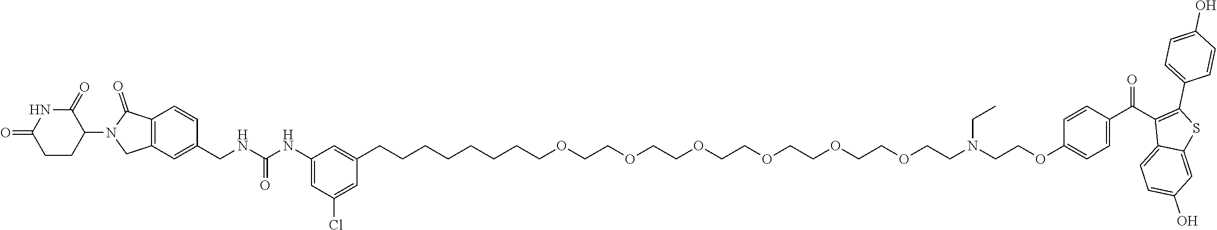 [SMILES: CCN(CCOCCOCCOCCOCCOCCOCCCCCCCCc1cc(Cl)cc(NC(=O)NCc2ccc3c(c2)CN(C2CCC(=O)NC2=O)C3=O)c1)CCOc1ccc(C(=O)c2c(-c3ccc(O)cc3)sc3cc(O)ccc23)cc1] |
| I-56 | 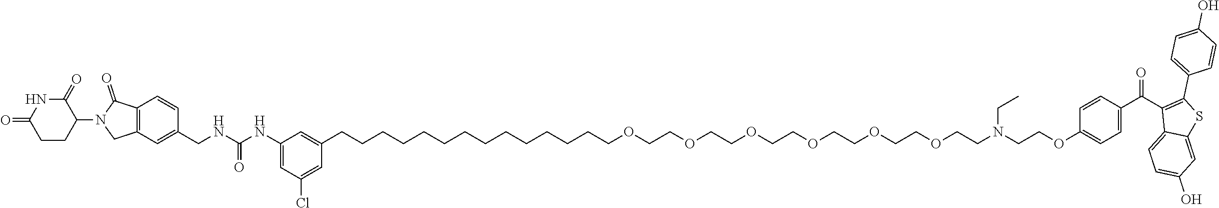 [SMILES: CCN(CCOCCOCCOCCOCCOCCOCCCCCCCCCCCCCCc1cc(Cl)cc(NC(=O)NCc2ccc3c(c2)CN(C2CCC(=O)NC2=O)C3=O)c1)CCOc1ccc(C(=O)c2c(-c3ccc(O)cc3)sc3cc(O)ccc23)cc1] |
| I-57 | 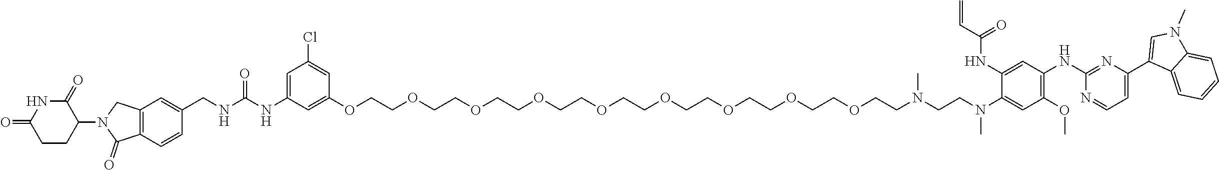 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCOc1cc(Cl)cc(NC(=O)NCc2ccc3c(c2)CN(C2CCC(=O)NC2=O)C3=O)c1] |
| I-58 | 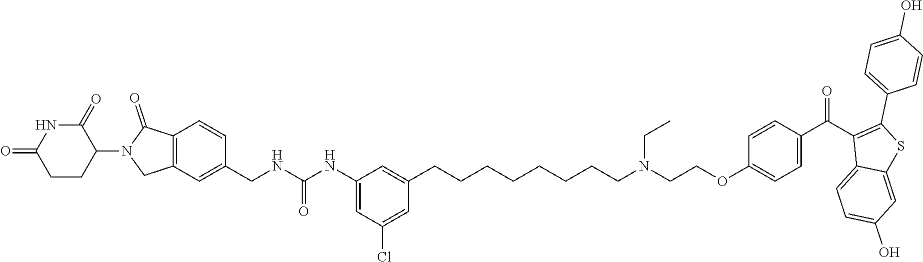 [SMILES: CCN(CCCCCCCCc1cc(Cl)cc(NC(=O)NCc2ccc3c(c2)CN(C2CCC(=O)NC2=O)C3=O)c1)CCOc1ccc(C(=O)c2c(-c3ccc(O)cc3)sc3cc(O)ccc23)cc1] |
| I-59 | 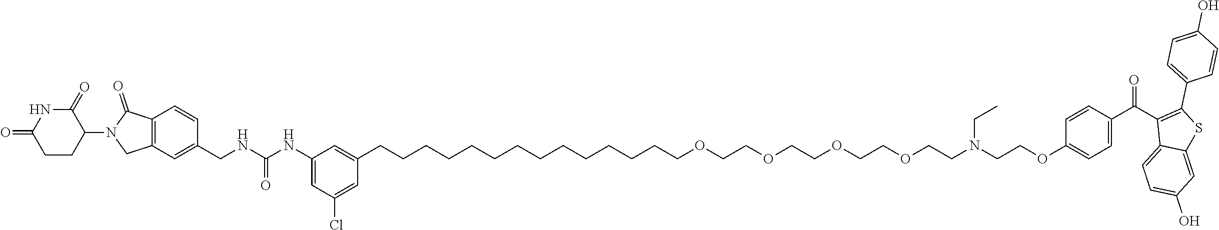 [SMILES: CCN(CCOCCOCCOCCOCCCCCCCCCCCCCCc1cc(Cl)cc(NC(=O)NCc2ccc3c(c2)CN(C2CCC(=O)NC2=O)C3=O)c1)CCOc1ccc(C(=O)c2c(-c3ccc(O)cc3)sc3cc(O)ccc23)cc1] |
| I-60 | 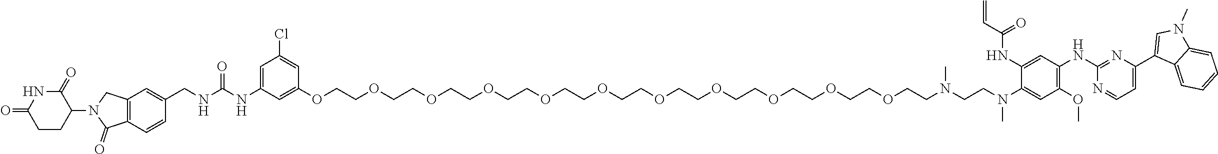 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCOCCOCCOc1cc(Cl)cc(NC(=O)NCc2ccc3c(c2)CN(C2CCC(=O)NC2=O)C3=O)c1] |
| I-61 | 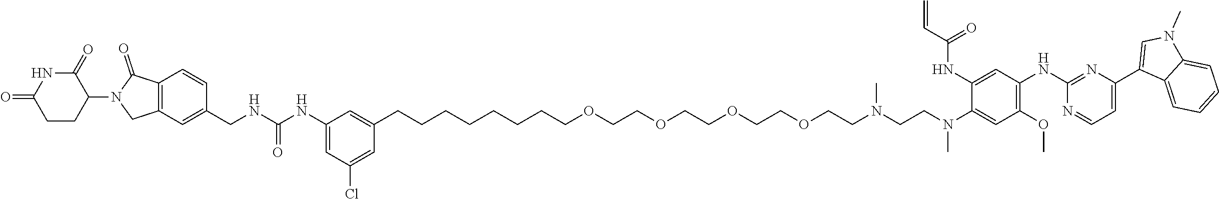 [SMILES: C=CC(=O)Nc1cc(Nc2nccc(-c3cn(C)c4ccccc34)n2)c(OC)cc1N(C)CCN(C)CCOCCOCCOCCOCCCCCCCCc1cc(Cl)cc(NC(=O)NCc2ccc3c(c2)CN(C2CCC(=O)NC2=O)C3=O)c1] |
| I-62 | 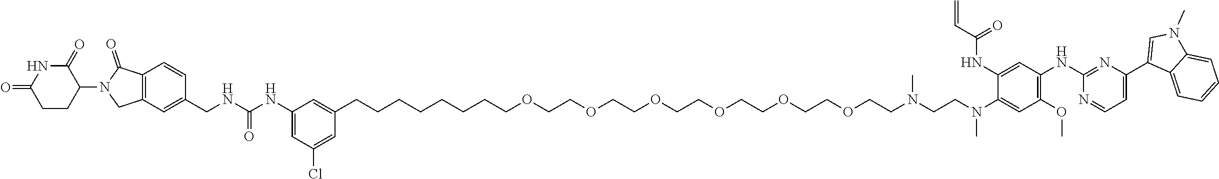 [SMILES: C=CC(=O)Nc1cc(Nc2nccc(-c3cn(C)c4ccccc34)n2)c(OC)cc1N(C)CCN(C)CCOCCOCCOCCOCCOCCOCCCCCCCCc1cc(Cl)cc(NC(=O)NCc2ccc3c(c2)CN(C2CCC(=O)NC2=O)C3=O)c1] |
| I-63 | 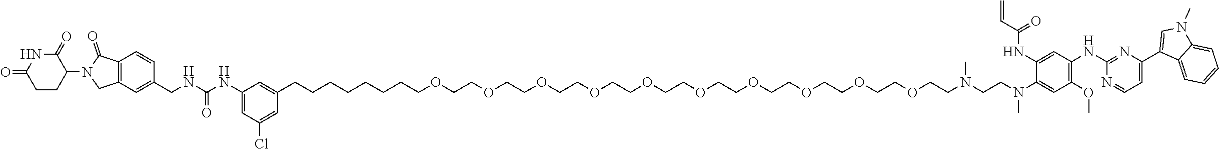 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-64 | 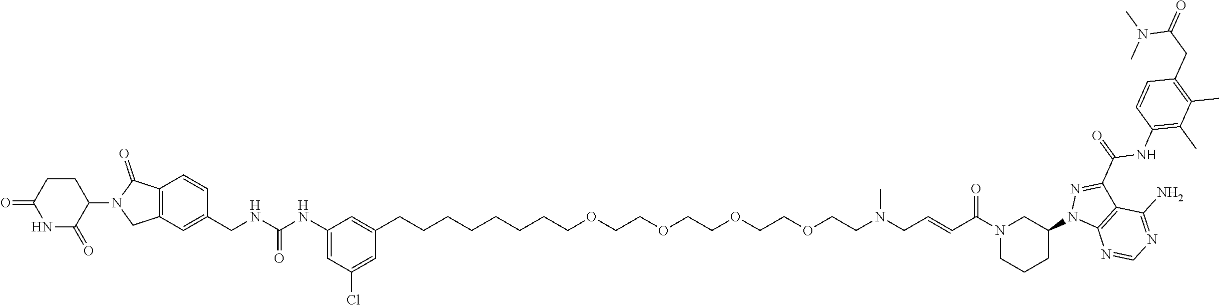 [SMILES: Cc1c(CC(=O)N(C)C)ccc(NC(=O)c2nn([C@H]3CCCN(C(=O)/C=C/CN(C)CCOCCOCCOCCOCCCCCCCCc4cc(Cl)cc(NC(=O)NCc5ccc6c(c5)CN(C5CCC(=O)NC5=O)C6=O)c4)C3)c3ncnc(N)c23)c1C] |
| I-65 | 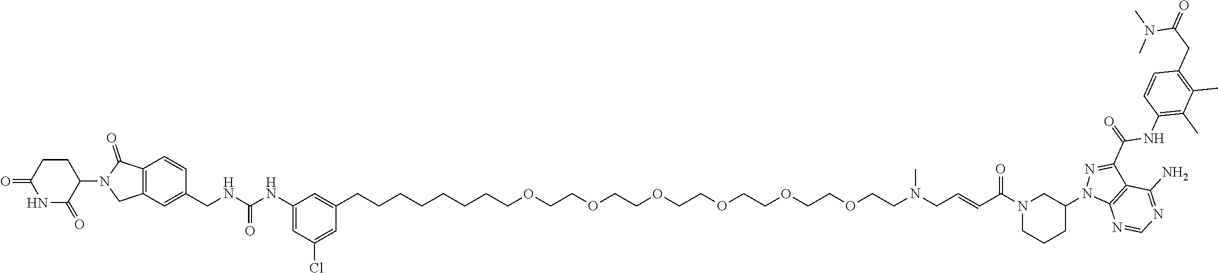 [SMILES: Cc1c(CC(=O)N(C)C)ccc(NC(=O)c2nn(C3CCCN(C(=O)/C=C/CN(C)CCOCCOCCOCCOCCOCCOCCCCCCCCc4cc(Cl)cc(NC(=O)NCc5ccc6c(c5)CN(C5CCC(=O)NC5=O)C6=O)c4)C3)c3ncnc(N)c23)c1C] |
| I-66 | 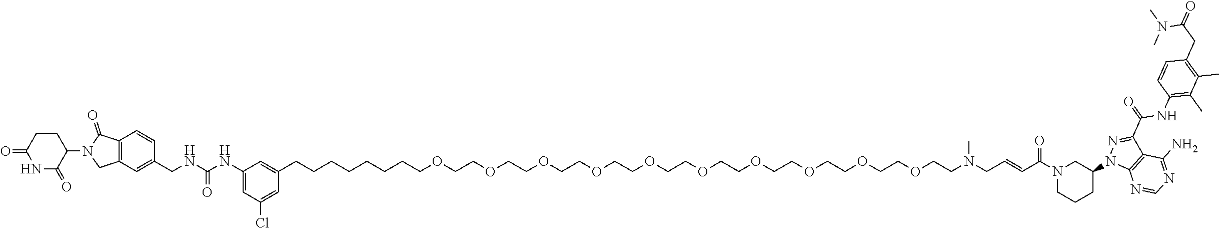 [SMILES: Cc1c(CC(=O)N(C)C)ccc(NC(=O)c2nn([C@H]3CCCN(C(=O)/C=C/CN(C)CCOCCOCCOCCOCCOCCOCCOCCOCCOCCOCCCCCCCCc4cc(Cl)cc(NC(=O)NCc5ccc6c(c5)CN(C5CCC(=O)NC5=O)C6=O)c4)C3)c3ncnc(N)c23)c1C] |
| I-67 | 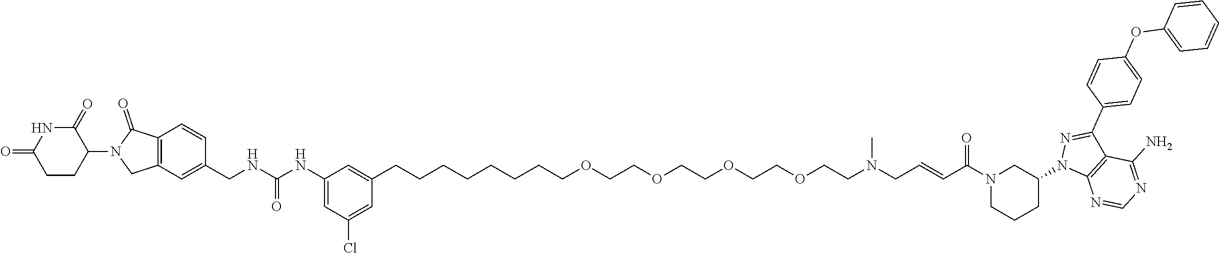 [SMILES: CN(C/C=C/C(=O)N1CCC[C@@H](n2nc(-c3ccc(Oc4ccccc4)cc3)c3c(N)ncnc32)C1)CCOCCOCCOCCOCCCCCCCCc1cc(Cl)cc(NC(=O)NCc2ccc3c(c2)CN(C2CCC(=O)NC2=O)C3=O)c1] |
| I-68 | 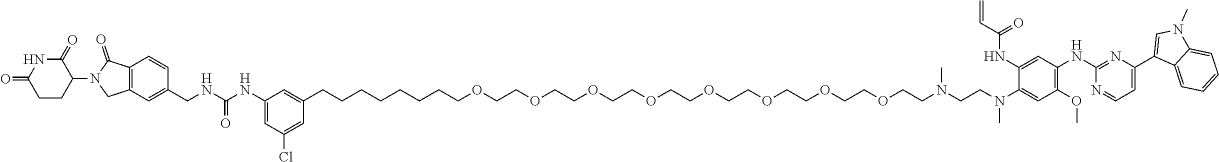 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-69 | 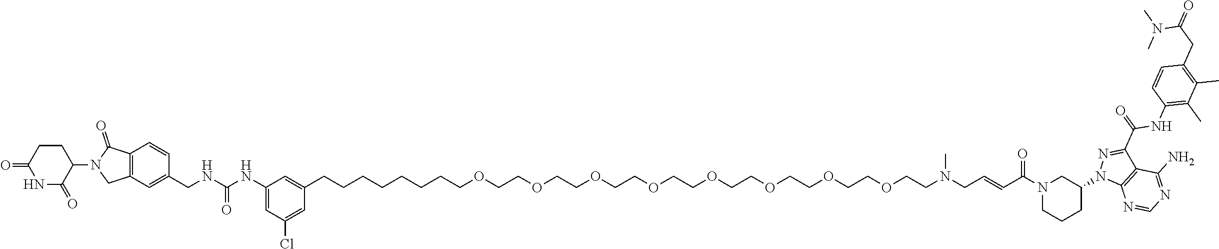 [SMILES: Cc1c(CC(=O)N(C)C)ccc(NC(=O)c2nn([C@@H]3CCCN(C(=O)/C=C/CN(C)CCOCCOCCOCCOCCOCCOCCOCCOCCCCCCCCc4cc(Cl)cc(NC(=O)NCc5ccc6c(c5)CN(C5CCC(=O)NC5=O)C6=O)c4)C3)c3ncnc(N)c23)c1C] |
| I-70 | 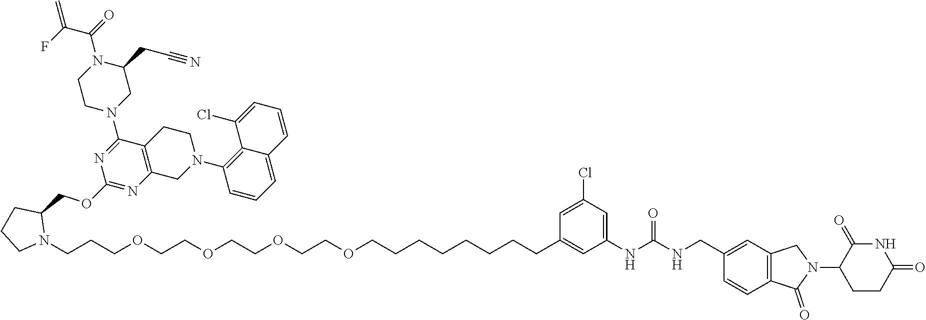 [SMILES: C=C(F)C(=O)N1CCN(c2nc(OC[C@@H]3CCCN3CCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-71 | 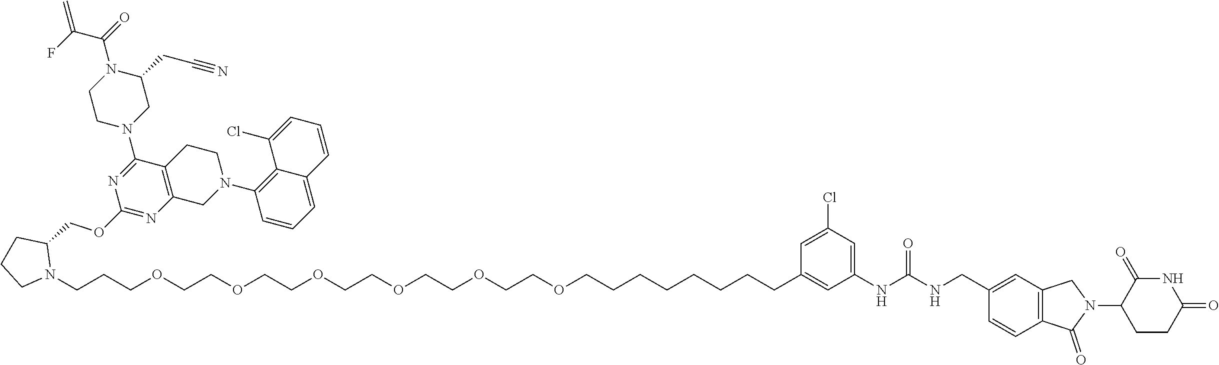 [SMILES: C=C(F)C(=O)N1CCN(c2nc(OC[C@H]3CCCN3CCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@H]1CC#N] |
| I-72 | 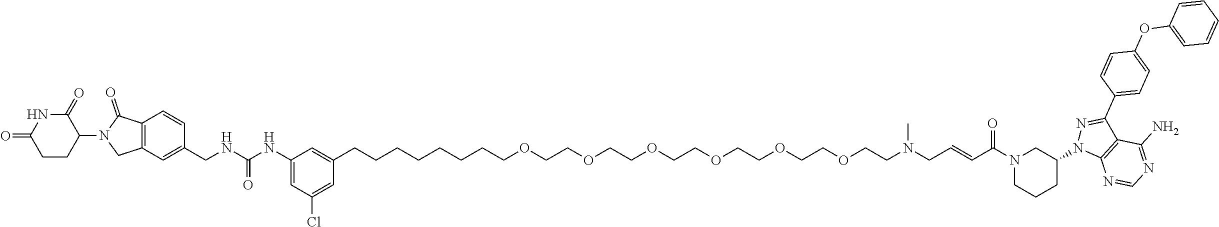 [SMILES: CN(C/C=C/C(=O)N1CCC[C@@H](n2nc(-c3ccc(Oc4ccccc4)cc3)c3c(N)ncnc32)C1)CCOCCOCCOCCOCCOCCOCCCCCCCCc1cc(Cl)cc(NC(=O)NCc2ccc3c(c2)CN(C2CCC(=O)NC2=O)C3=O)c1] |
| I-73 | 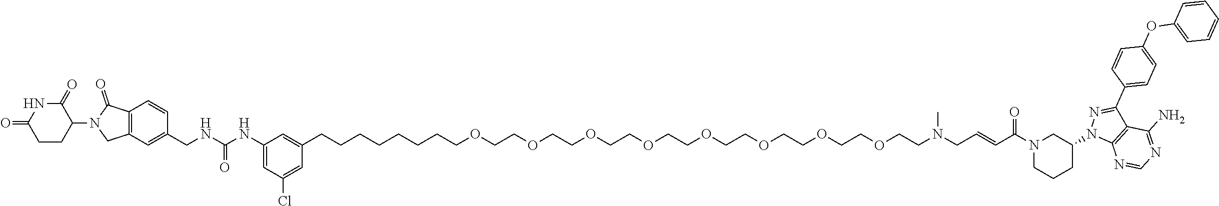 [SMILES: CN(C/C=C/C(=O)N1CCC[C@@H](n2nc(-c3ccc(Oc4ccccc4)cc3)c3c(N)ncnc32)C1)CCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-74 | 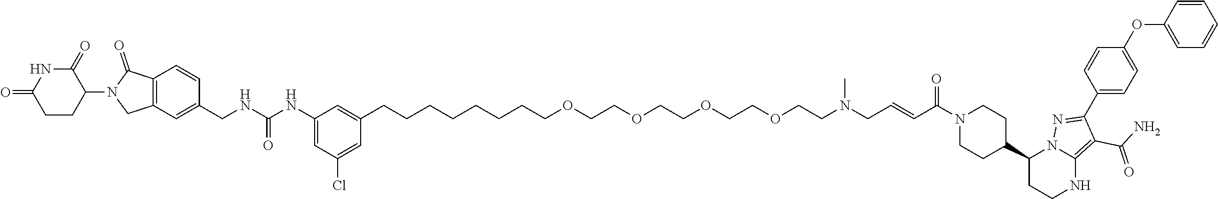 [SMILES: CN(C/C=C/C(=O)N1CCC([C@@H]2CCNc3c(C(N)=O)c(-c4ccc(Oc5ccccc5)cc4)nn32)CC1)CCOCCOCCOCCOCCCCCCCCc1cc(Cl)cc(NC(=O)NCc2ccc3c(c2)CN(C2CCC(=O)NC2=O)C3=O)c1] |
| I-75 | 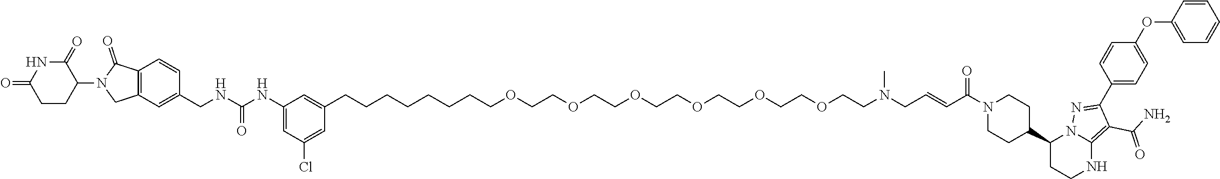 [SMILES: CN(C/C=C/C(=O)N1CCC([C@@H]2CCNc3c(C(N)=O)c(-c4ccc(Oc5ccccc5)cc4)nn32)CC1)CCOCCOCCOCCOCCOCCOCCCCCCCCc1cc(Cl)cc(NC(=O)NCc2ccc3c(c2)CN(C2CCC(=O)NC2=O)C3=O)c1] |
| I-76 | 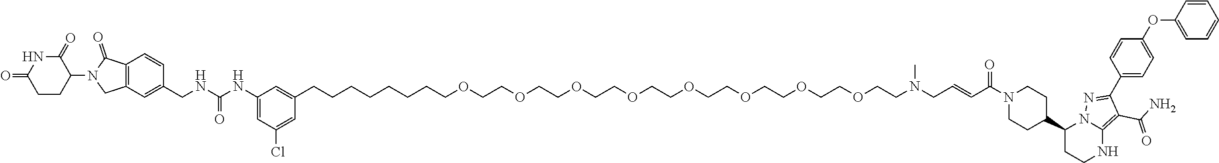 [SMILES: CN(C/C=C/C(=O)N1CCC([C@@H]2CCNc3c(C(N)=O)c(-c4ccc(Oc5ccccc5)cc4)nn32)CC1)CCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-77 | 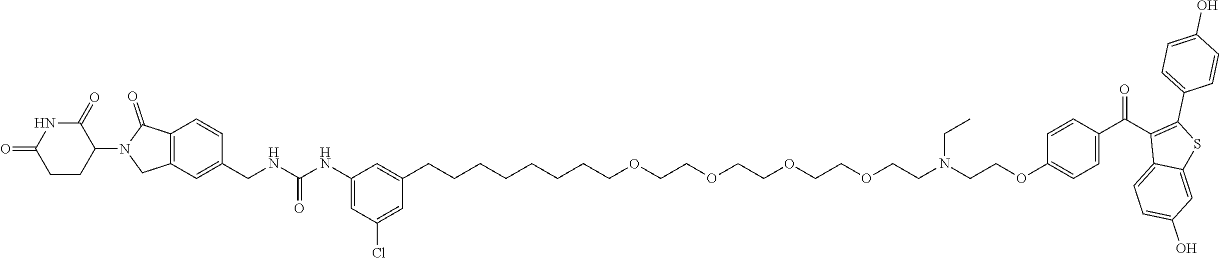 [SMILES: CCN(CCOCCOCCOCCOCCCCCCCCc1cc(Cl)cc(NC(=O)NCc2ccc3c(c2)CN(C2CCC(=O)NC2=O)C3=O)c1)CCOc1ccc(C(=O)c2c(-c3ccc(O)cc3)sc3cc(O)ccc23)cc1] |
| I-78 | 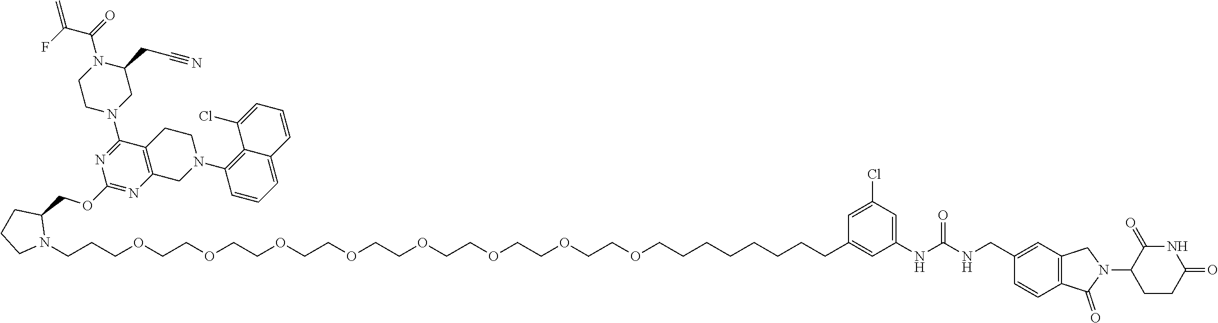 [SMILES: C=C(F)C(=O)N1CCN(c2nc(OC[C@@H]3CCCN3CCCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-80 | 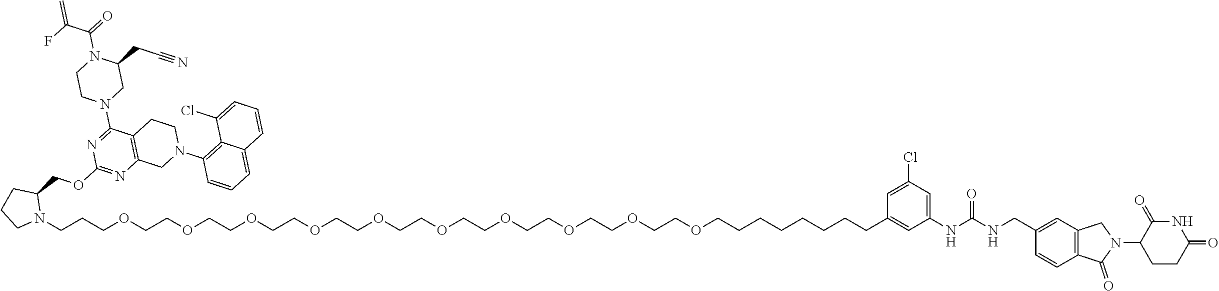 [SMILES: C=C(F)C(=O)N1CCN(c2nc(OC[C@@H]3CCCN3CCCOCCOCCOCCOCCOCCOCCOCCOCCOCCOCCCCCCCCc3cc(Cl)cc(NC(=O)NCc4ccc5c(c4)CN(C4CCC(=O)NC4=O)C5=O)c3)nc3c2CCN(c2cccc4cccc(Cl)c24)C3)C[C@@H]1CC#N] |
| I-81 | 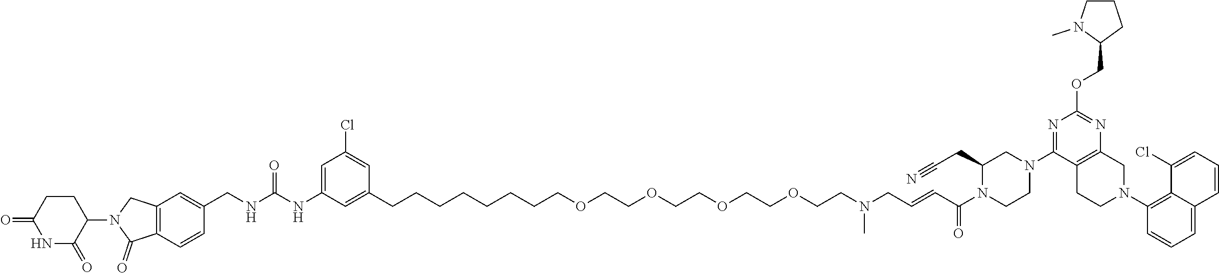 [SMILES: CN(C/C=C/C(=O)N1CCN(c2nc(OC[C@@H]3CCCN3C)nc3c2CCN(c2cccc4cccc(Cl)c24)C3)C[C@@H]1CC#N)CCOCCOCCOCCOCCCCCCCCc1cc(Cl)cc(NC(=O)NCc2ccc3c(c2)CN(C2CCC(=O)NC2=O)C3=O)c1] |
| I-82 | 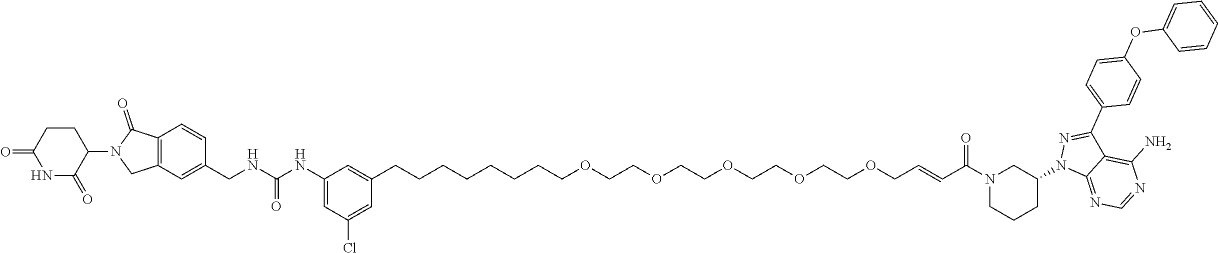 [SMILES: Nc1ncnc2c1c(-c1ccc(Oc3ccccc3)cc1)nn2[C@@H]1CCCN(C(=O)/C=C/COCCOCCOCCOCCOCCCCCCCCc2cc(Cl)cc(NC(=O)NCc3ccc4c(c3)CN(C3CCC(=O)NC3=O)C4=O)c2)C1] |
| I-83 | 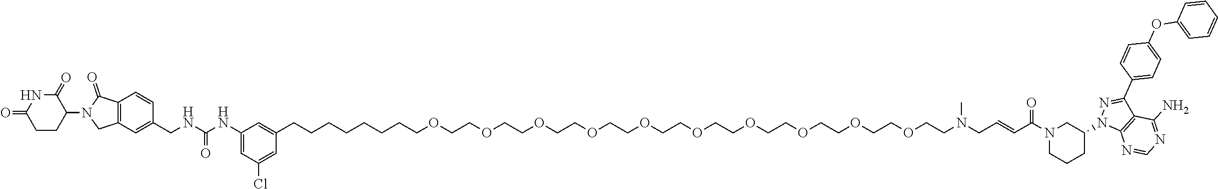 [SMILES: CN(C/C=C/C(=O)N1CCC[C@@H](n2nc(-c3ccc(Oc4ccccc4)cc3)c3c(N)ncnc32)C1)CCOCCOCCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-84 | 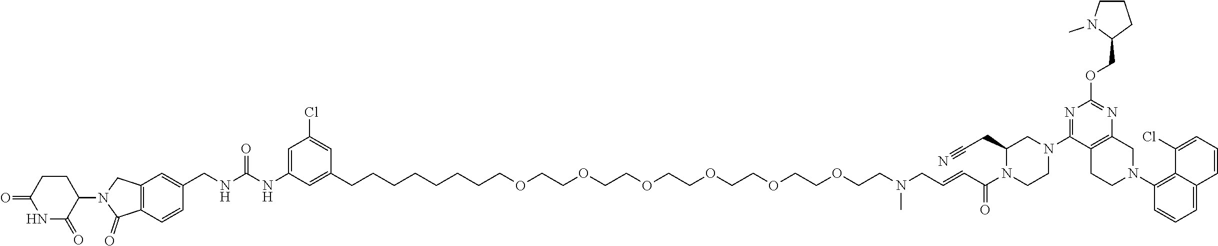 [SMILES: CN(C/C=C/C(=O)N1CCN(c2nc(OC[C@@H]3CCCN3C)nc3c2CCN(c2cccc4cccc(Cl)c24)C3)C[C@@H]1CC#N)CCOCCOCCOCCOCCOCCOCCCCCCCCc1cc(Cl)cc(NC(=O)NCc2ccc3c(c2)CN(C2CCC(=O)NC2=O)C3=O)c1] |
| I-85 | 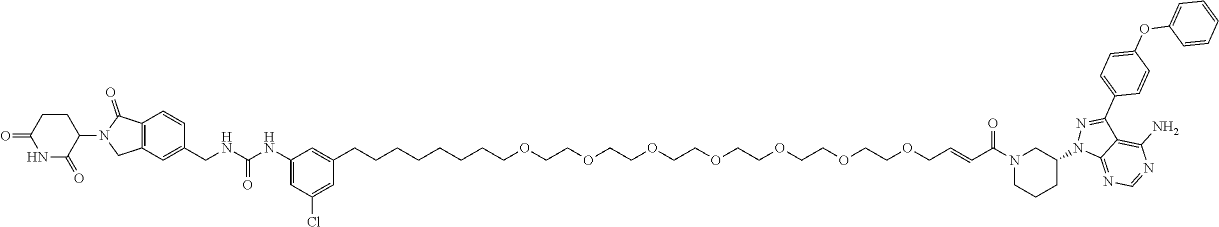 [SMILES: Nc1ncnc2c1c(-c1ccc(Oc3ccccc3)cc1)nn2[C@@H]1CCCN(C(=O)/C=C/COCCOCCOCCOCCOCCOCCOCCCCCCCCc2cc(Cl)cc(NC(=O)NCc3ccc4c(c3)CN(C3CCC(=O)NC3=O)C4=O)c2)C1] |
| I-86 | 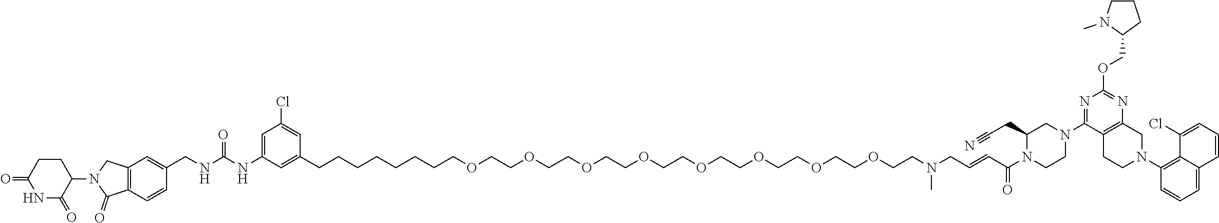 [SMILES: CN(C/C=C/C(=O)N1CCN(c2nc(OC[C@H]3CCCN3C)nc3c2CCN(c2cccc4cccc(Cl)c24)C3)C[C@@H]1CC#N)CCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-87 | 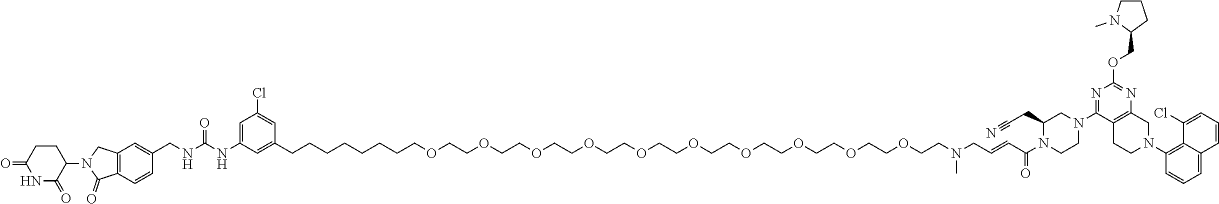 [SMILES: CN(C/C=C/C(=O)N1CCN(c2nc(OC[C@@H]3CCCN3C)nc3c2CCN(c2cccc4cccc(Cl)c24)C3)C[C@@H]1CC#N)CCOCCOCCOCCOCCOCCOCCOCCOCCOCCOCCCCCCCCc1cc(Cl)cc(NC(=O)NCc2ccc3c(c2)CN(C2CCC(=O)NC2=O)C3=O)c1] |
| I-88 | 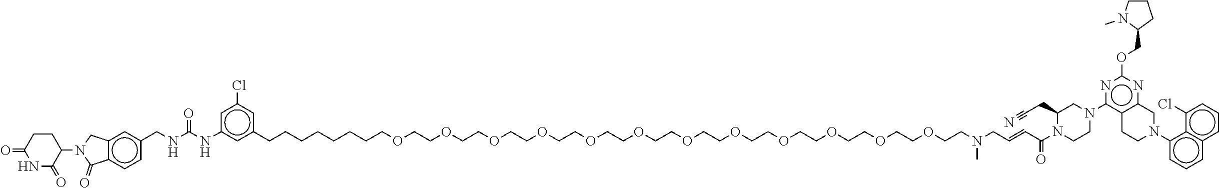 |
| I-89 | 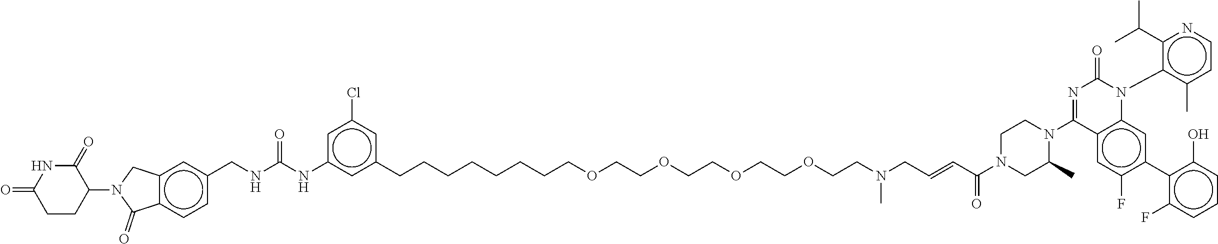 |
| I-90 | 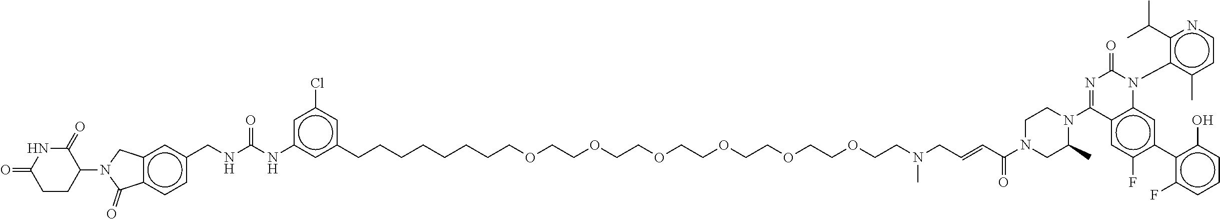 |
| I-91 | 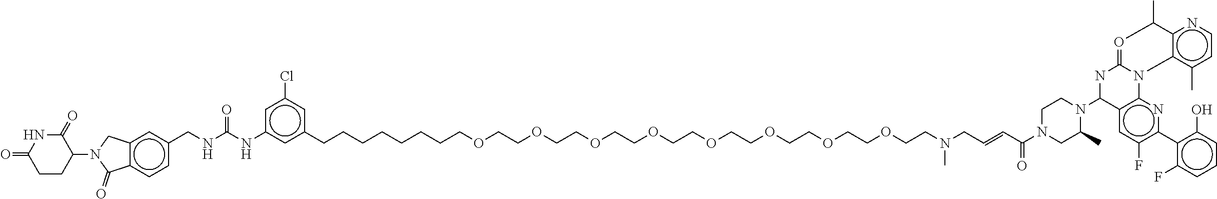 |
| I-92 | 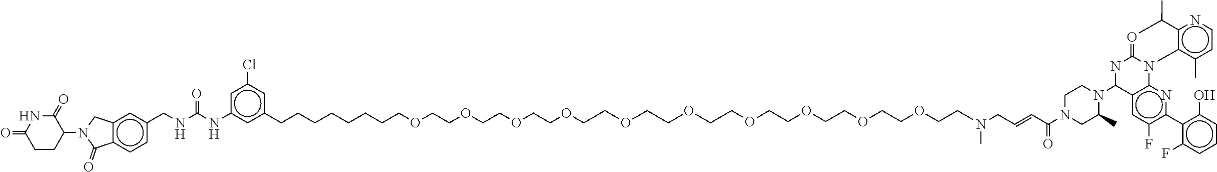 |
| I-93 | 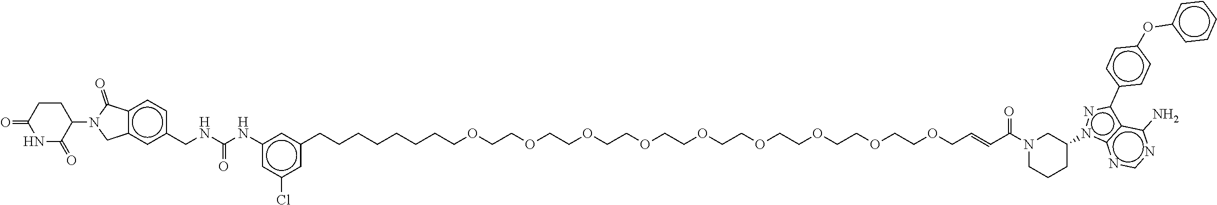 |
| I-94 | 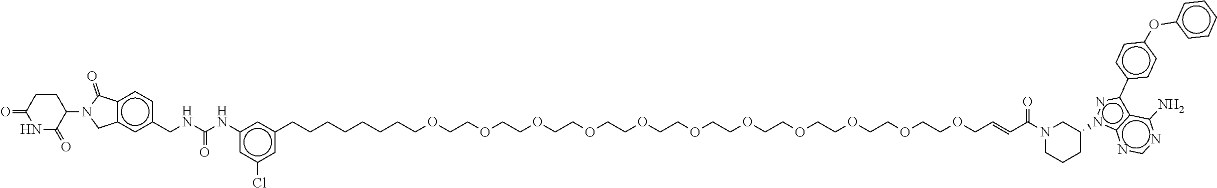 |
| I-95 | 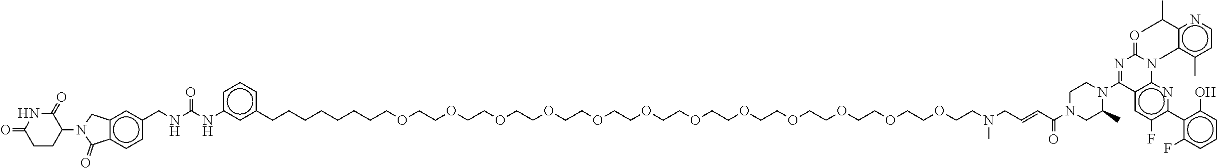 |
| I-96 | 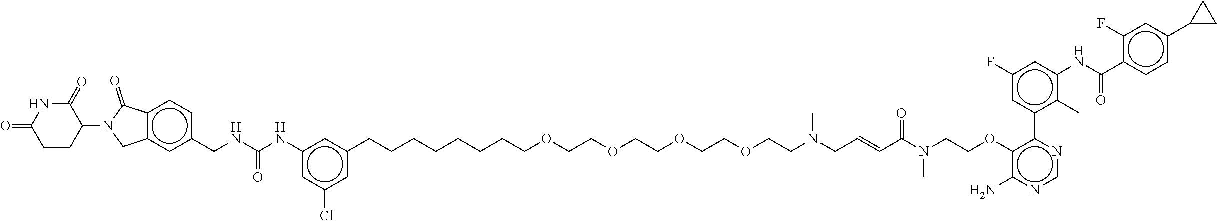 |
| I-97 | 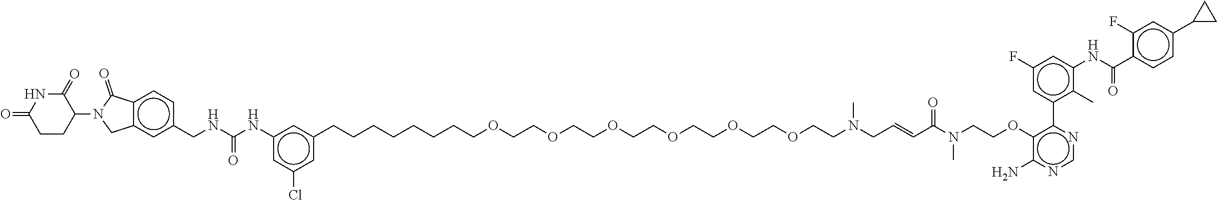 |
| I-98 | 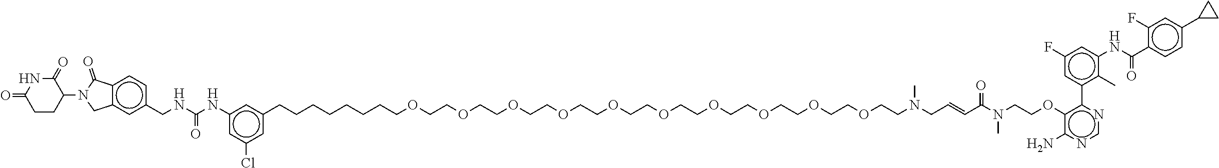 |
| I-99 | 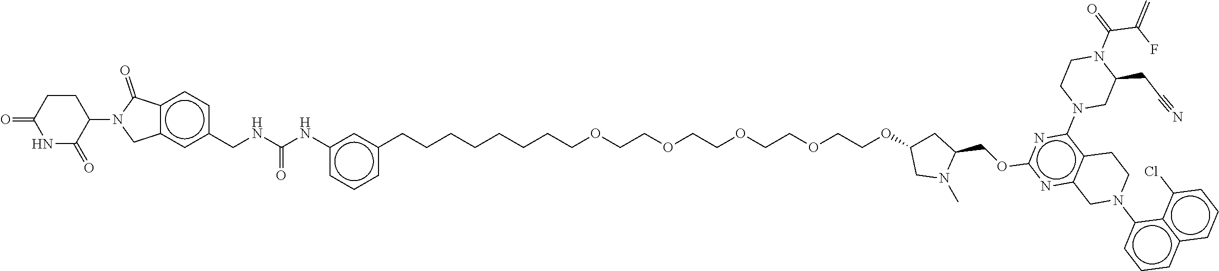 |
| I-100 | 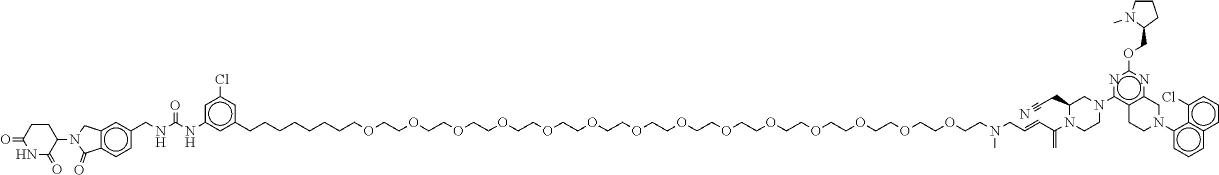 |
| I-101 | 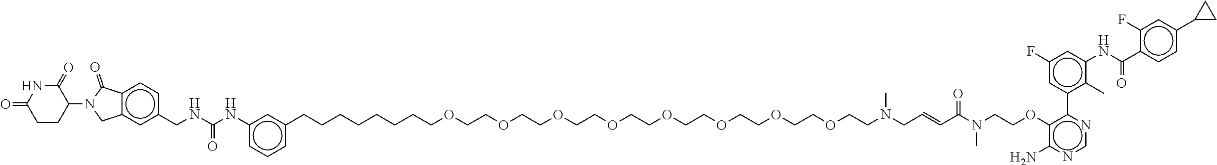 |
| I-102 | 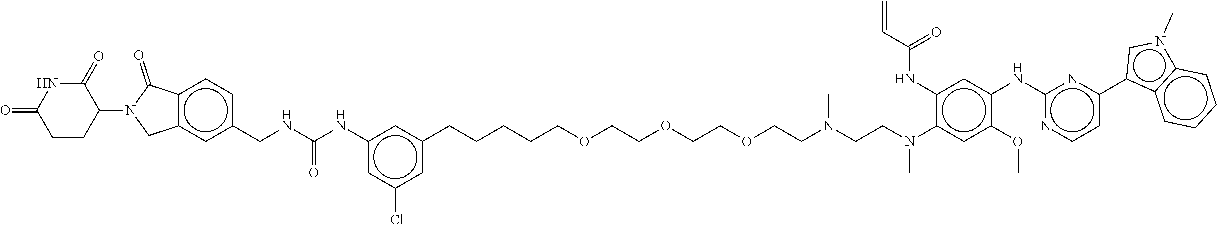 |
| I-103 | 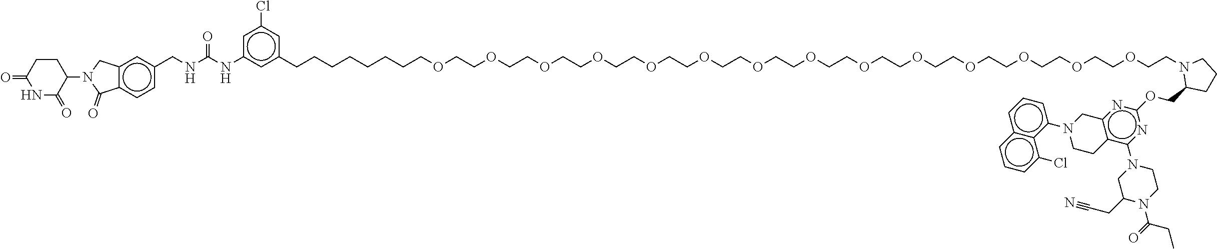 |
| I-104 | 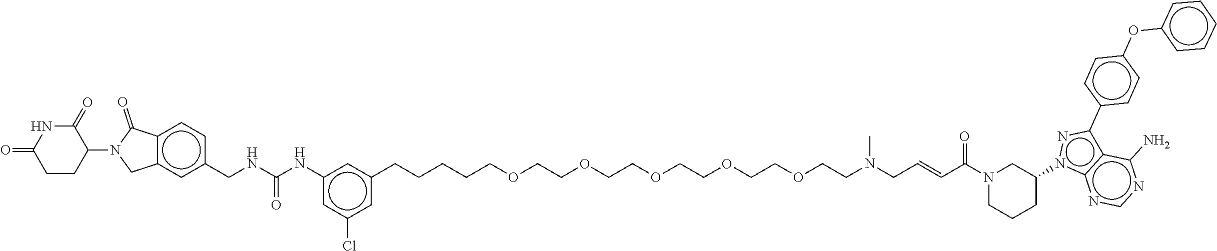 |
| I-105 | 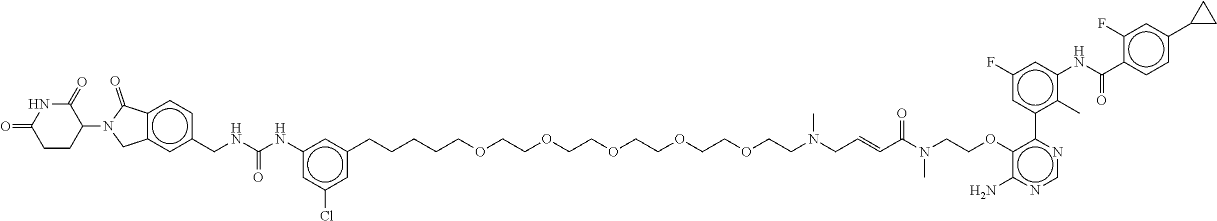 |
| I-106 | 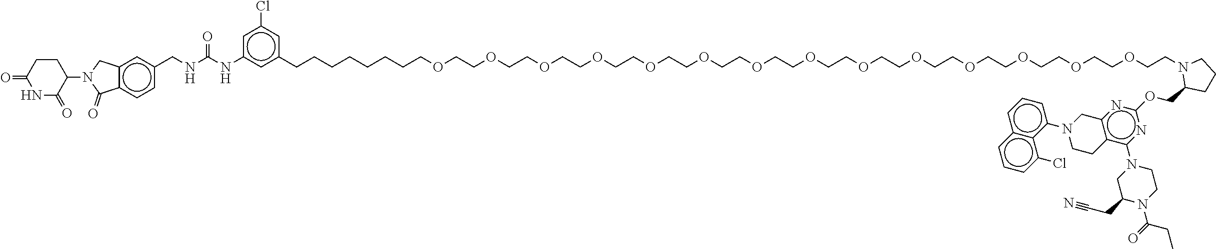 |
| I-107 | 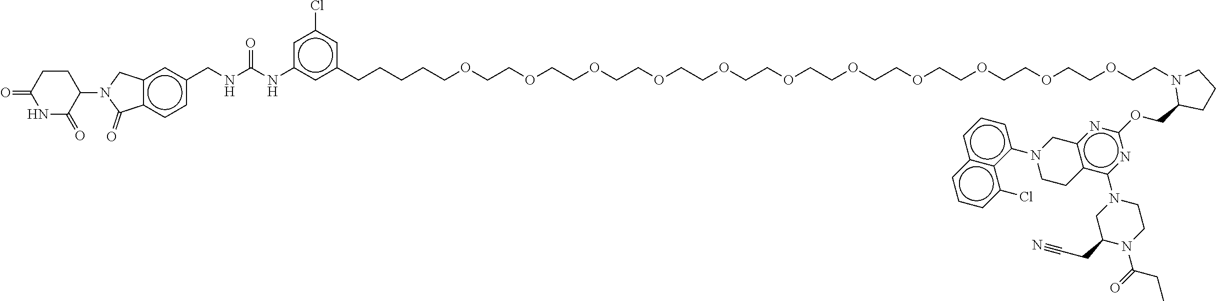 |
| I-108 | 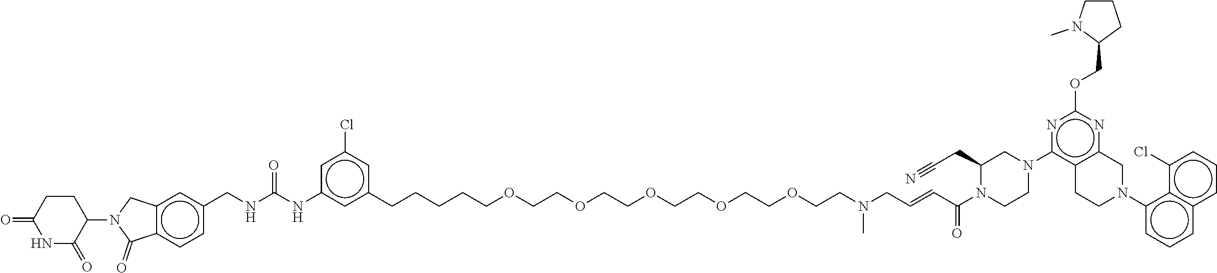 |
| I-109 | 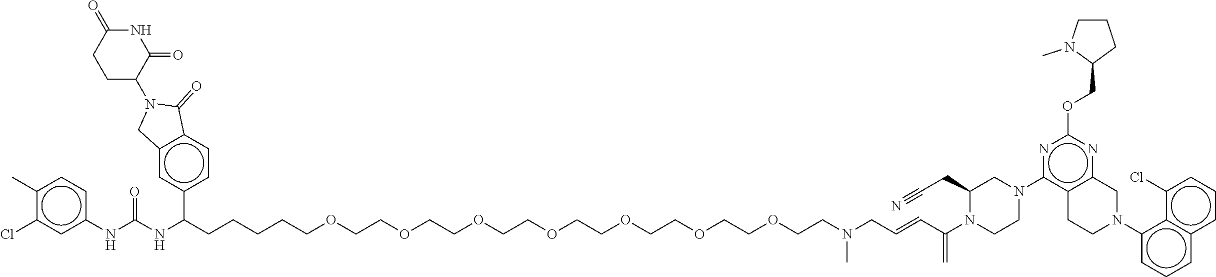 |
| I-110 | 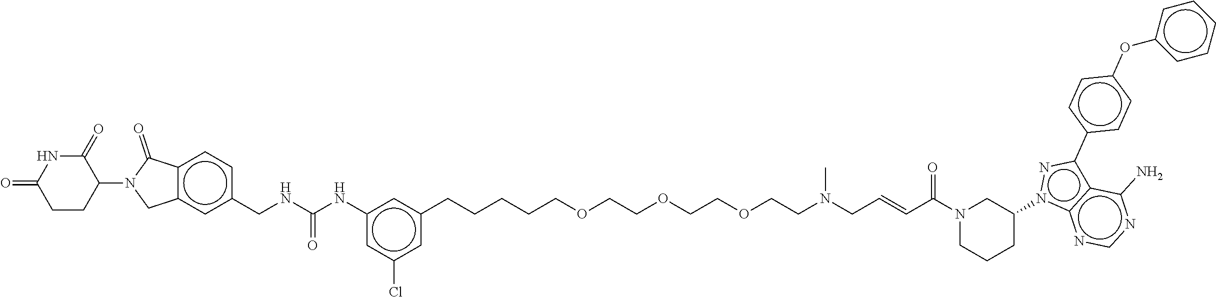 |
| I-111 | 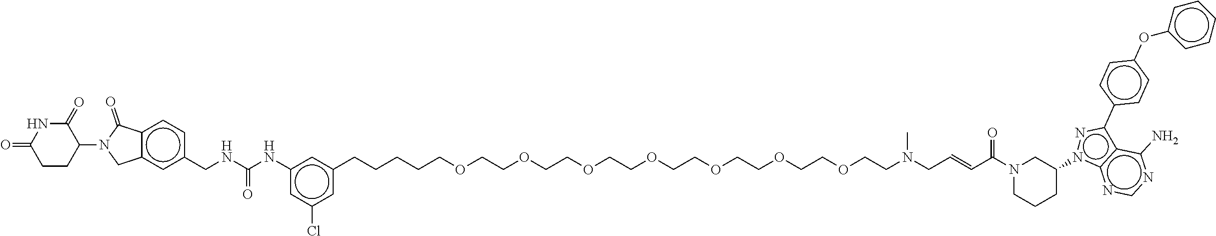 |
| I-112 | 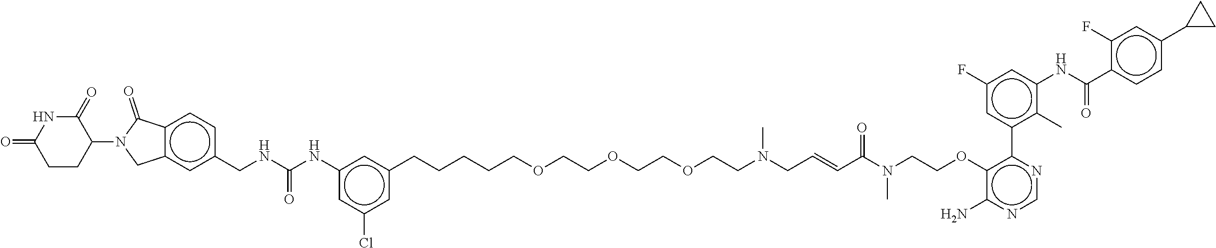 |
| I-113 | 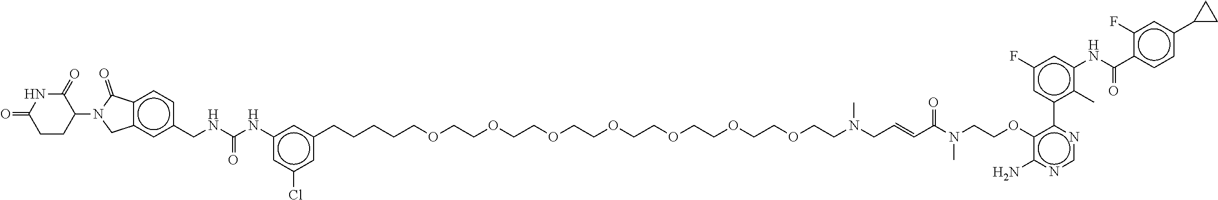 |
| I-114 | 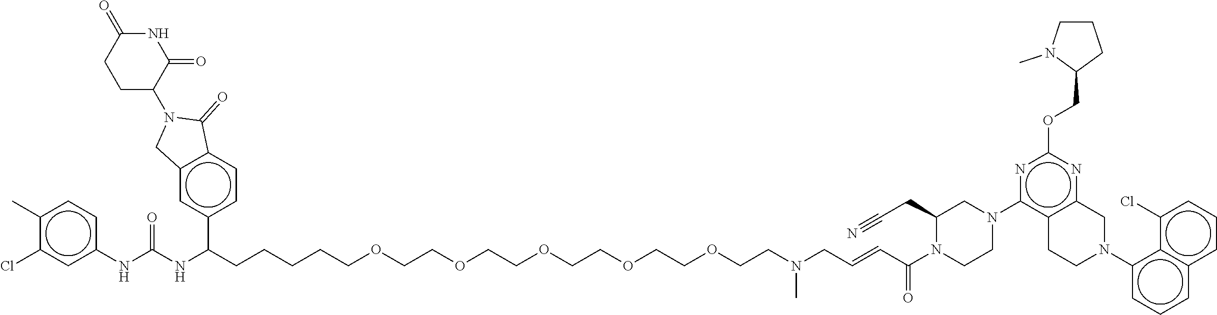 |
| I-115 | 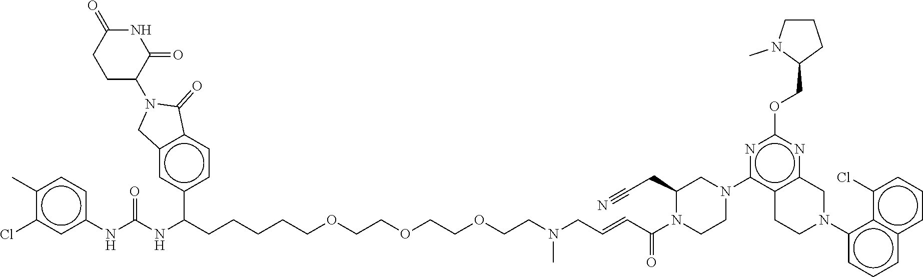 |
| I-116 | 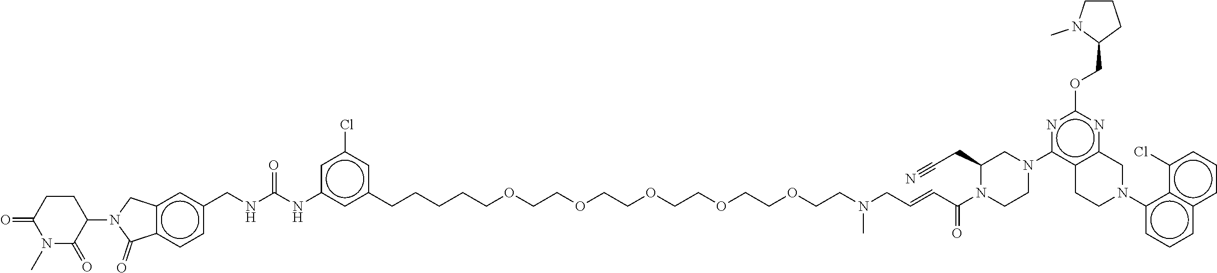 |
| I-117 | 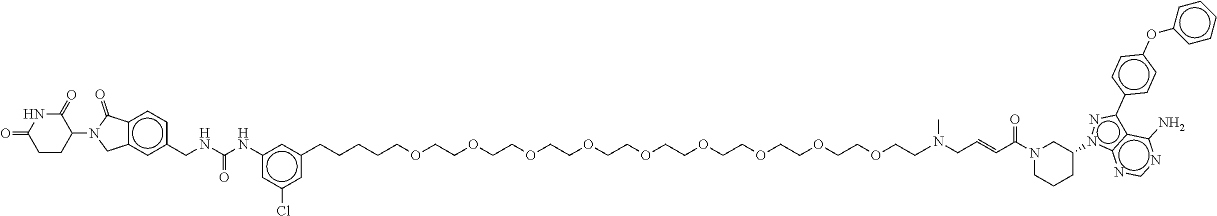 |
| I-118 | 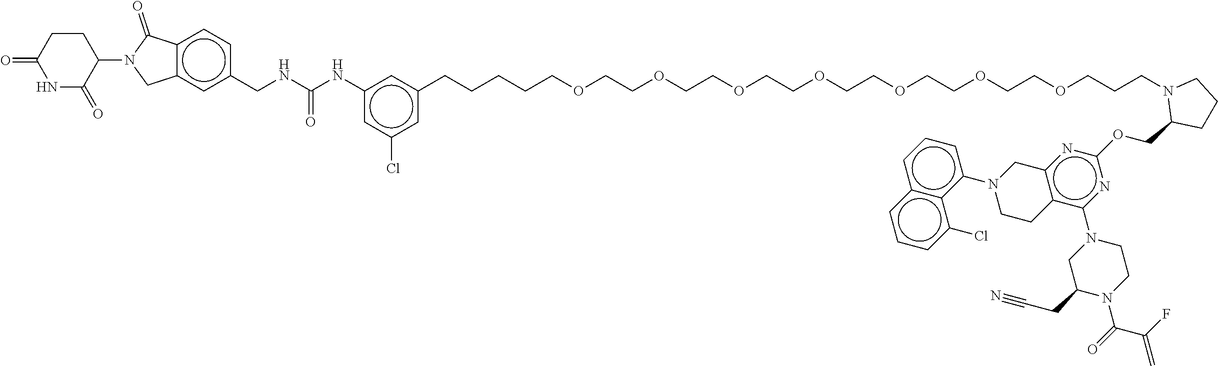 |
| I-119 | 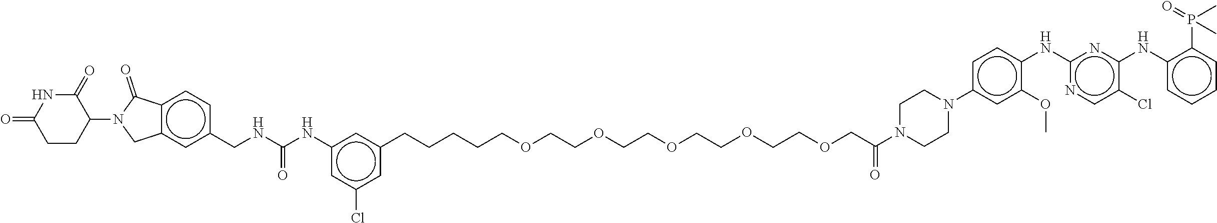 |
| I-120 | 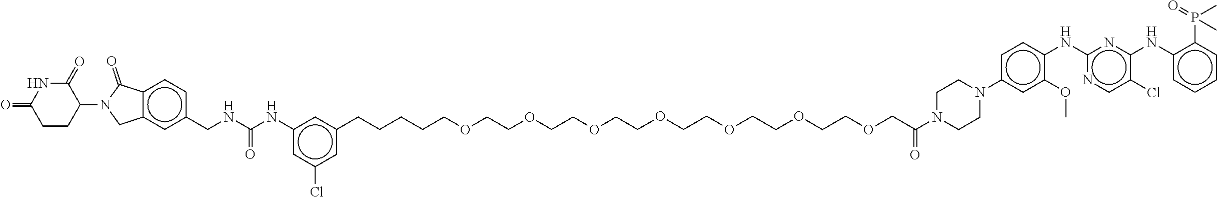 |
| I-121 | 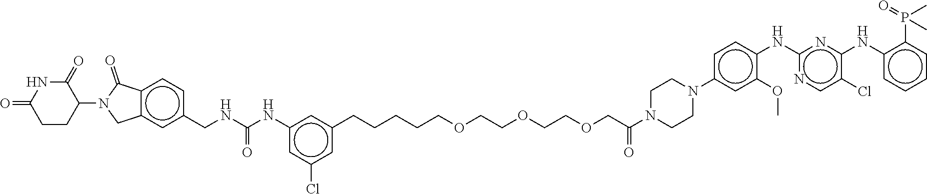 |
| I-122 | 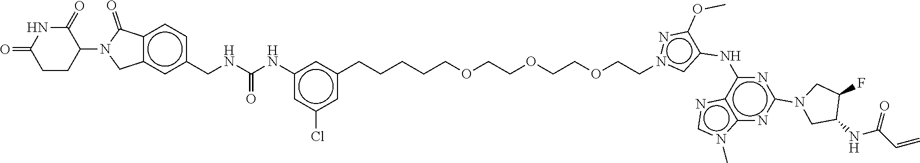 |
| I-123 | 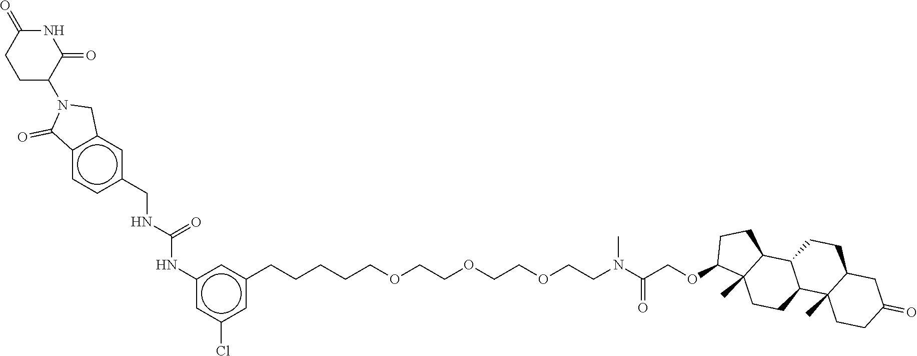 |
| I-124 | 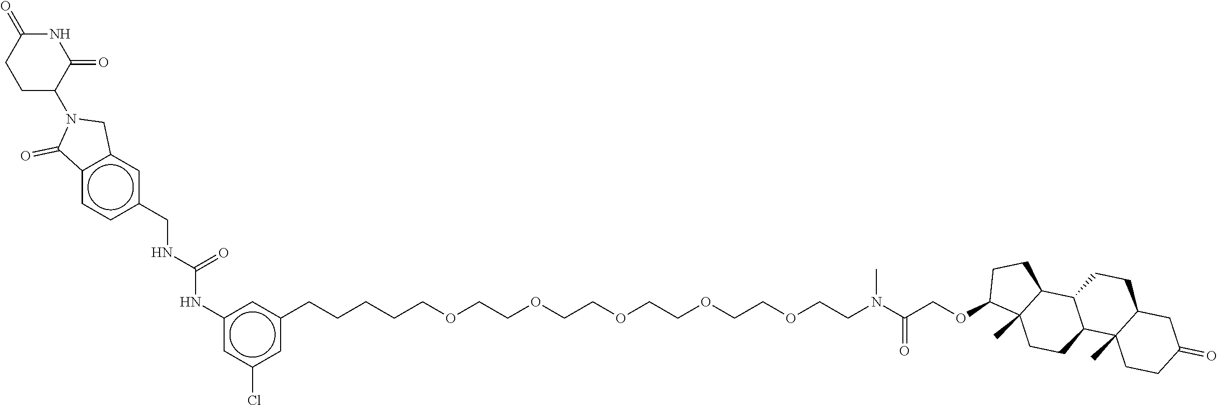 |
| I-125 | 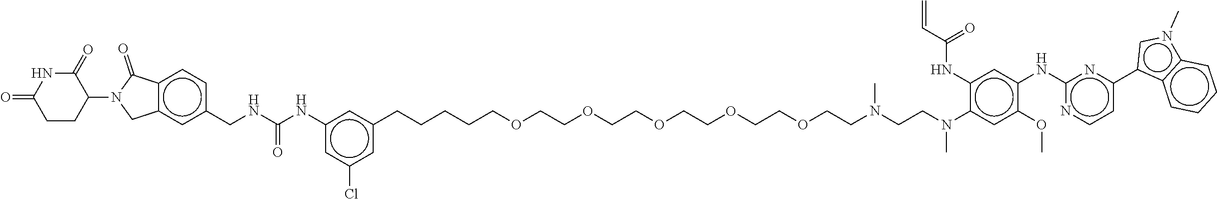 |
| I-126 | 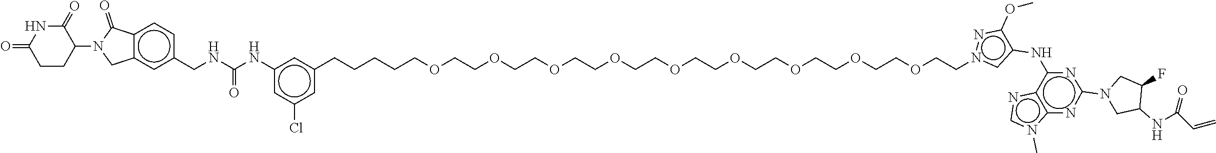 |
| I-127 | 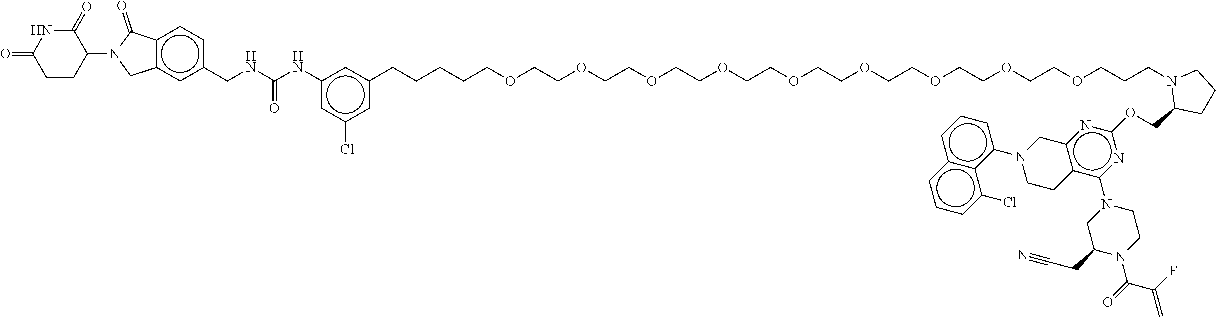 |
| I-128 | 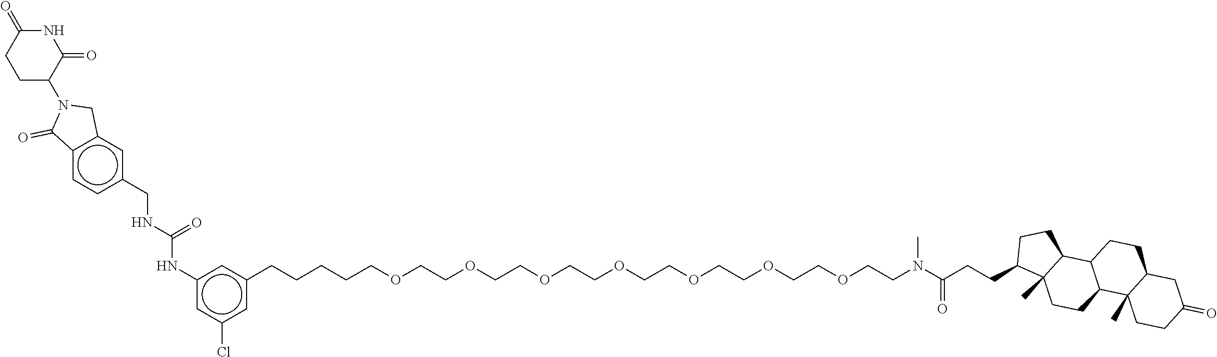 |
| I-129 | 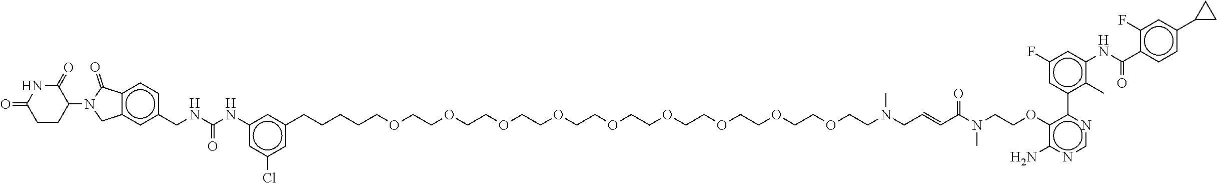 |
| I-130 | 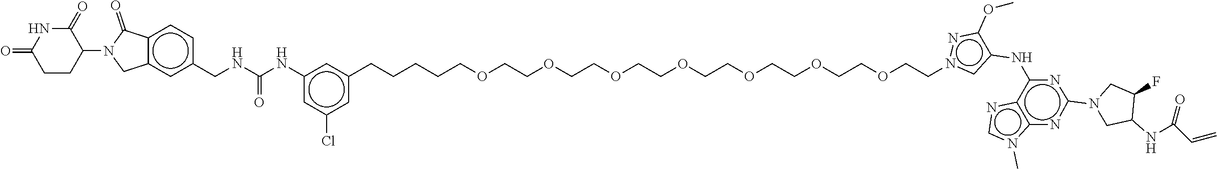 |
| I-131 | 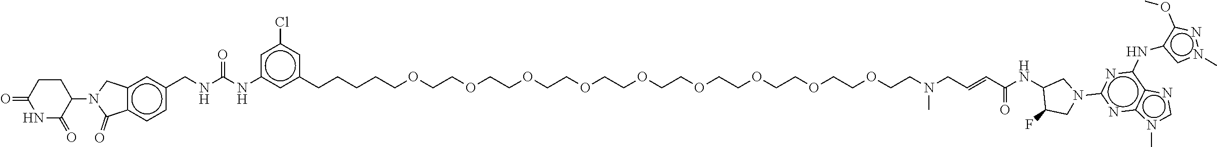 |
| I-132 | 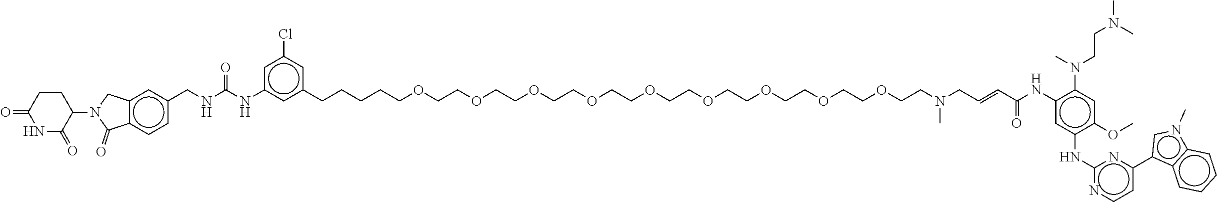 |
| I-133 | 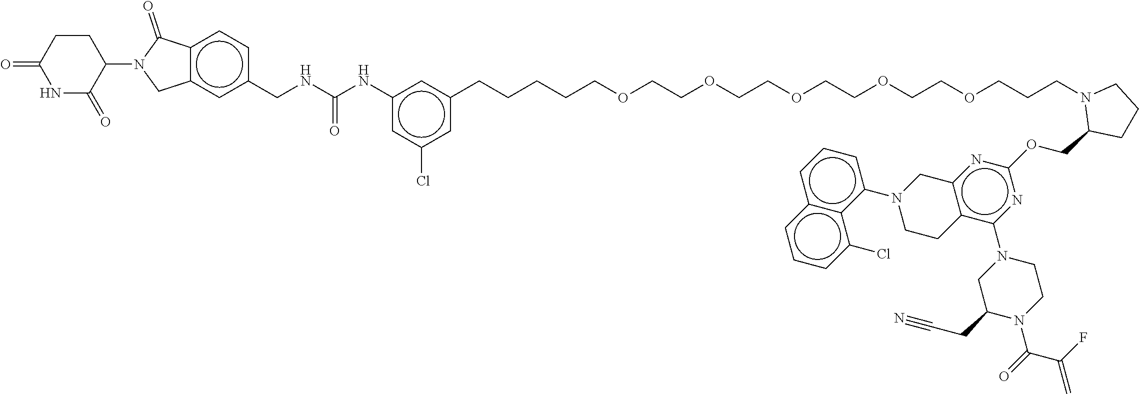 |
| I-134 | 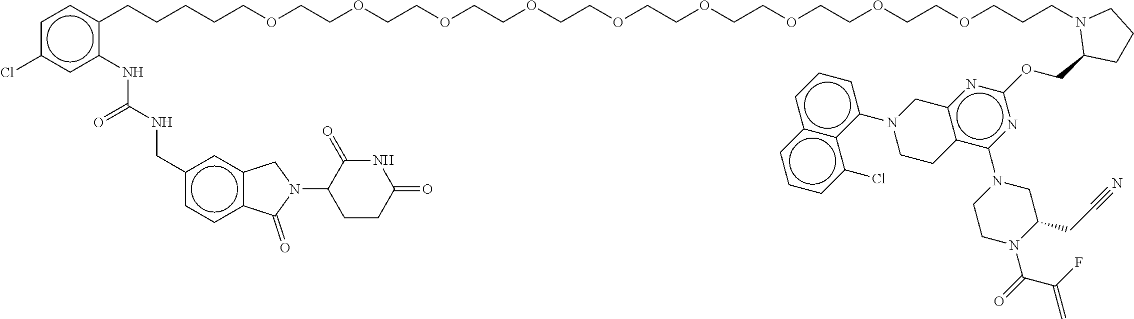 |
| I-135 | 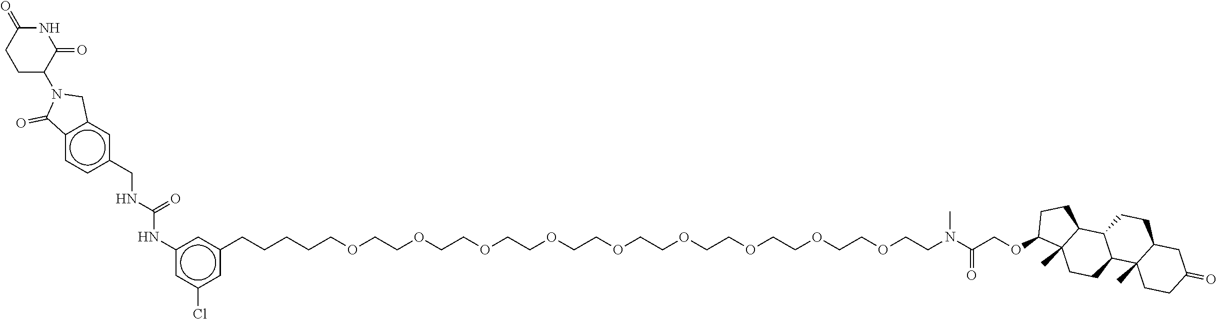 |
| I-136 | 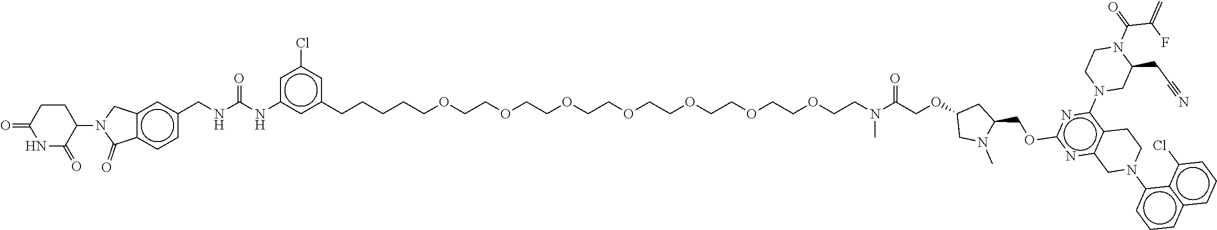 |
| I-137 | 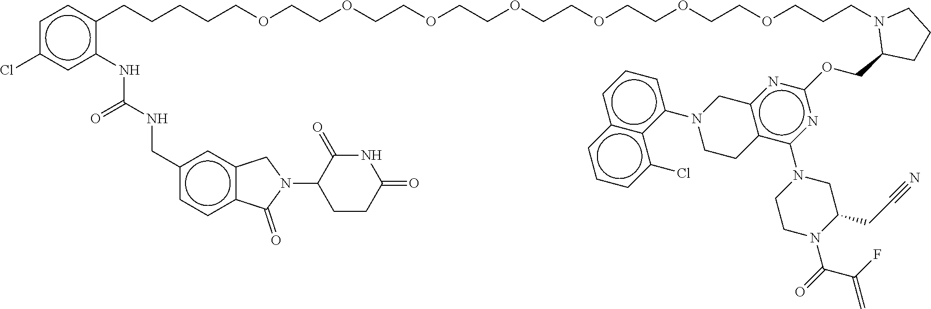 |
| I-138 | 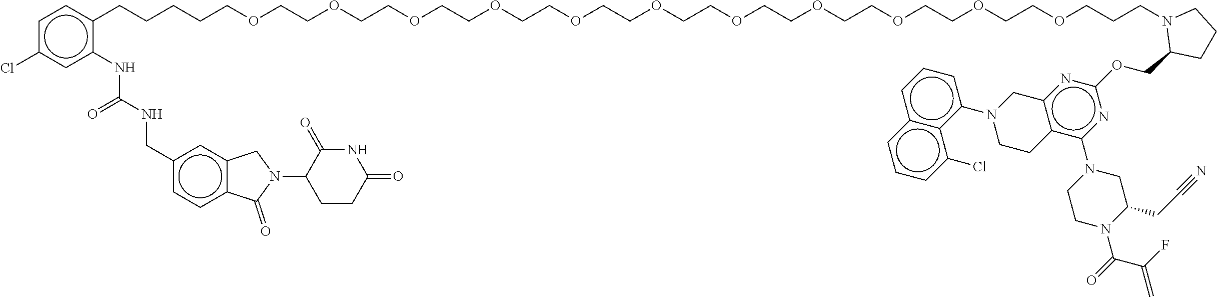 |
| I-139 | 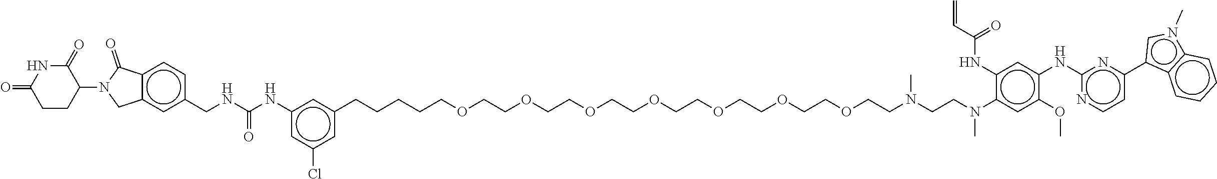 |
| I-140 | 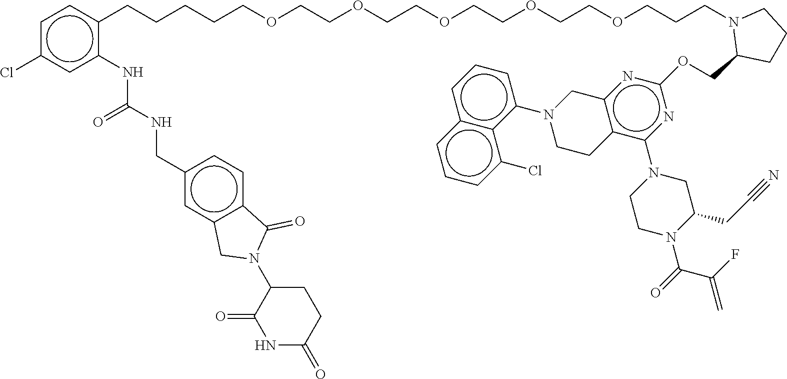 |
| I-141 | 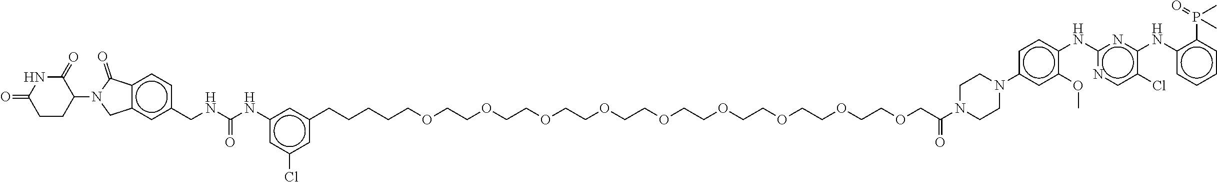 |
| I-142 | 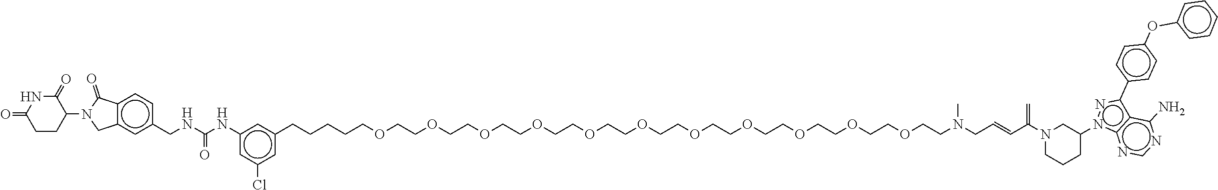 |
| I-143 | 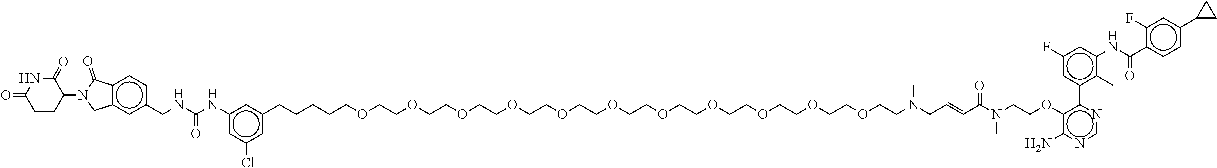 |
| I-144 | 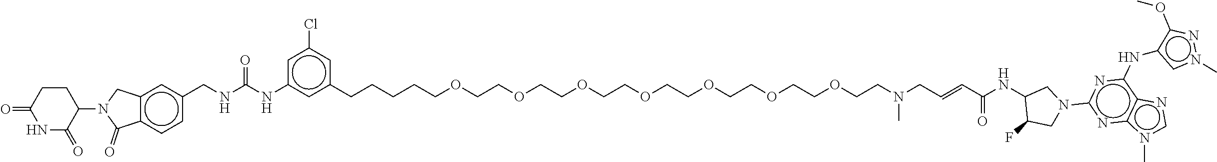 |
| I-145 | 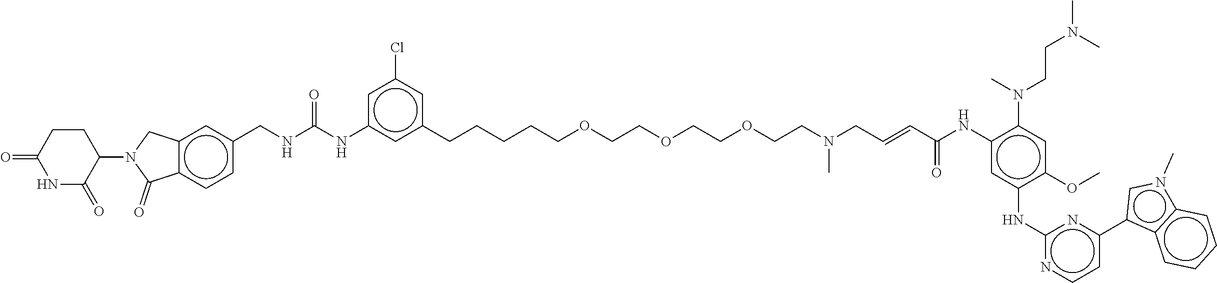 |
| I-146 | 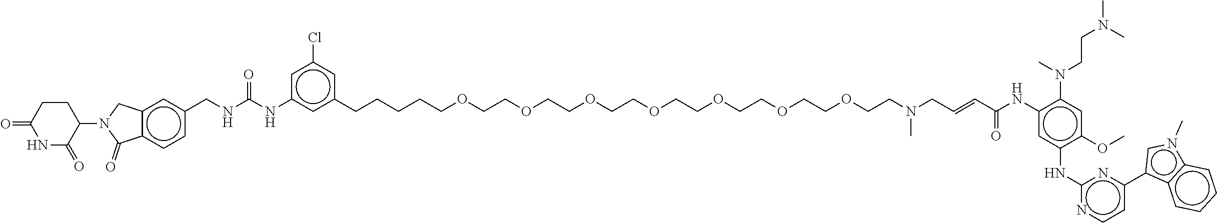 |
| I-147 | 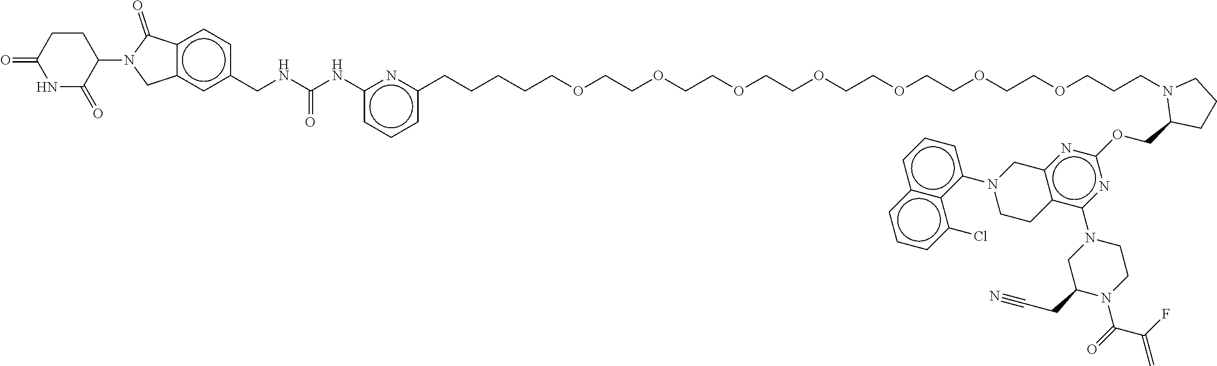 |
| I-148 | 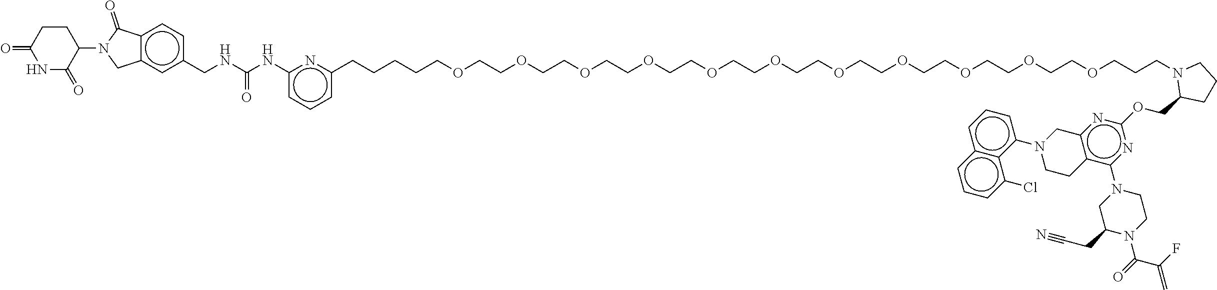 |
| I-149 | 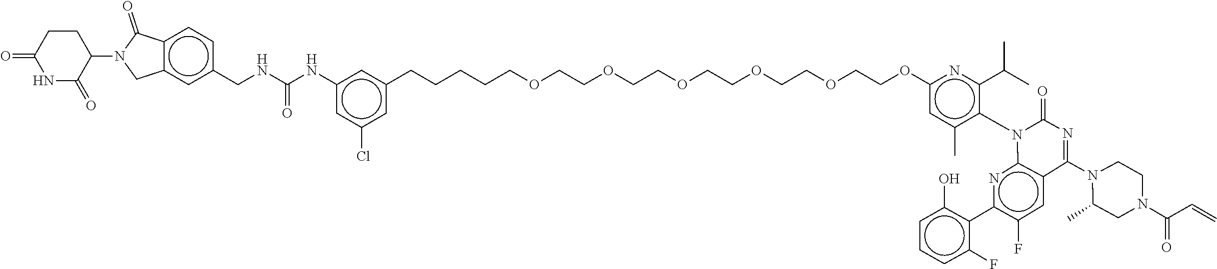 |
| I-150 | 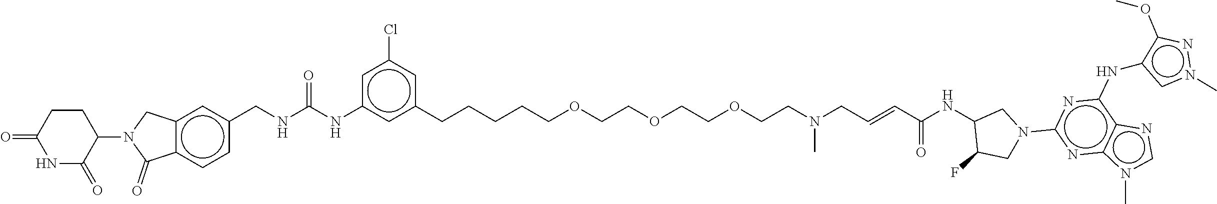 |
| I-151 | 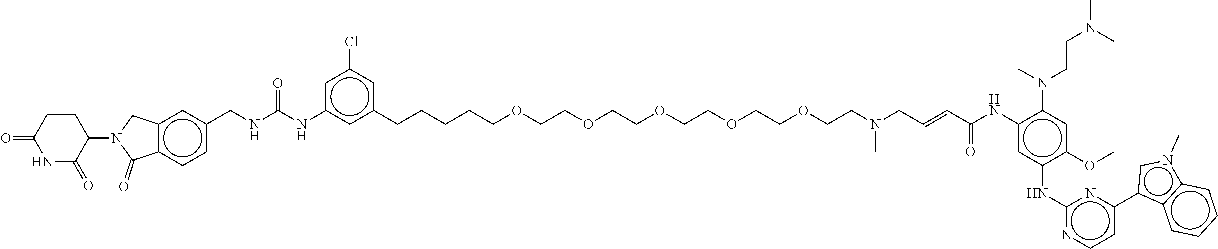 |
| I-152 | 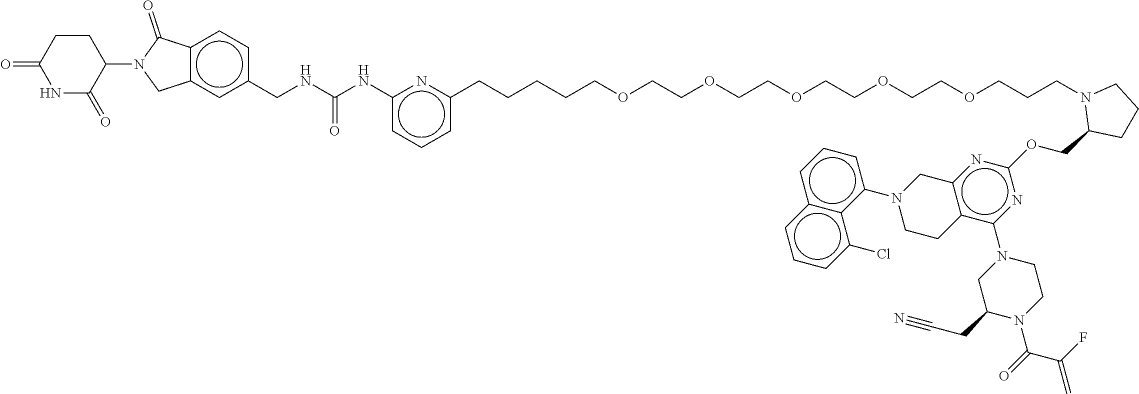 |
| I-153 | 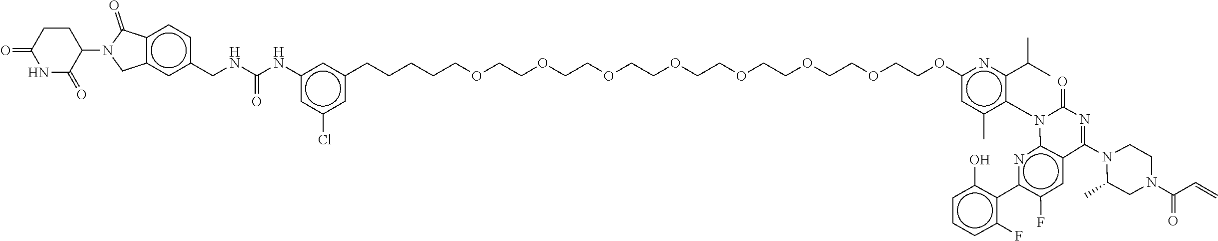 |
| I-154 | 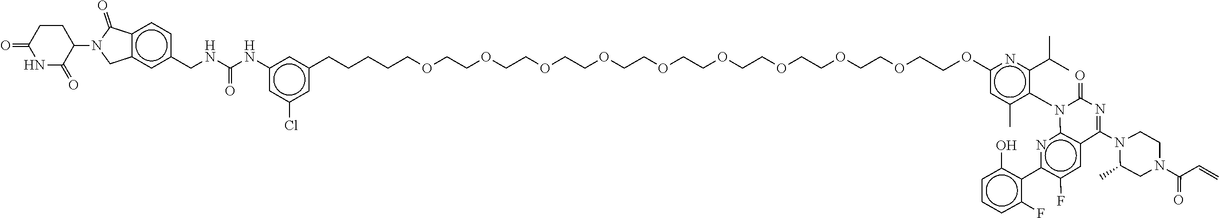 |
| I-155 | 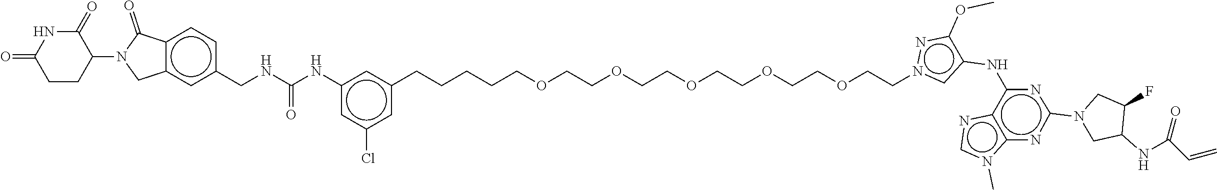 |
| I-156 | 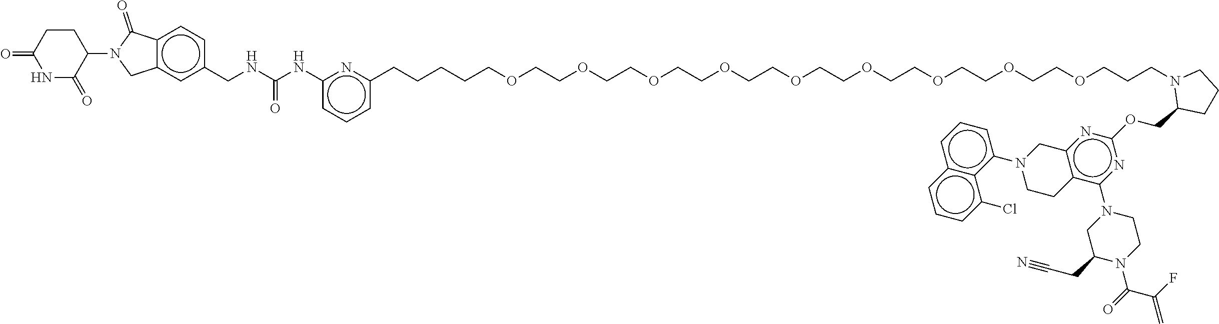 |
| I-157 | 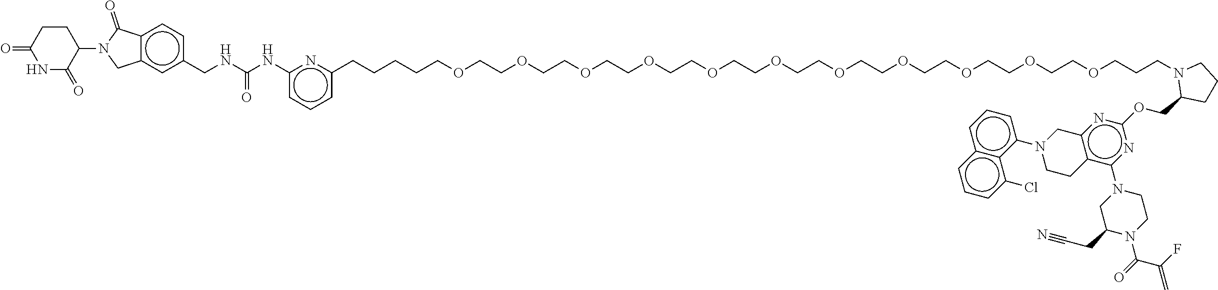 |
| I-158 | 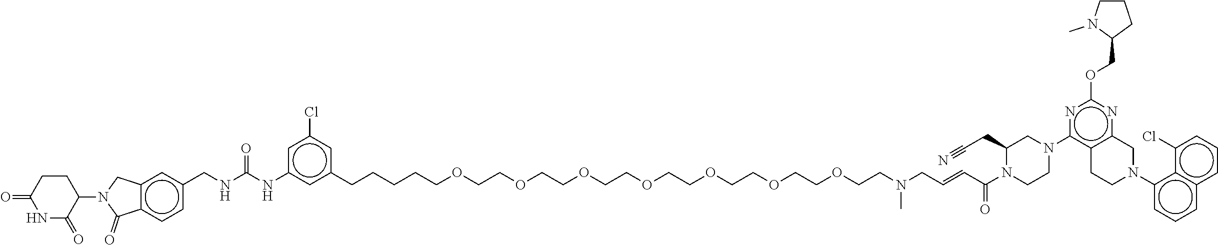 |
| I-159 | 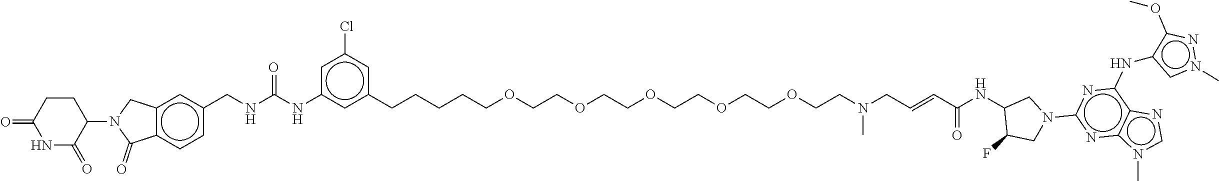 |
| I-160 | 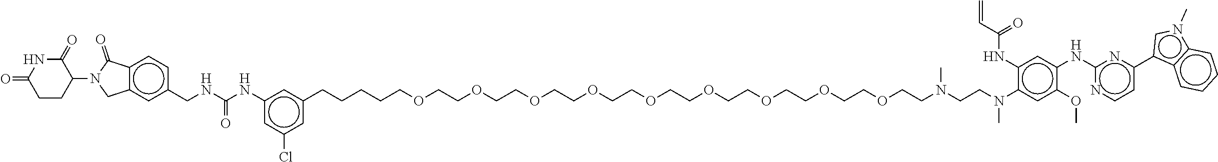 |
| I-161 | 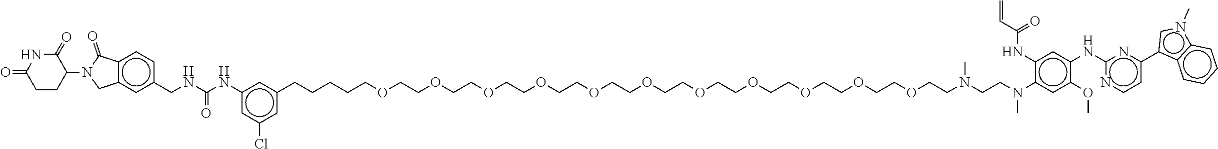 |
| I-162 | 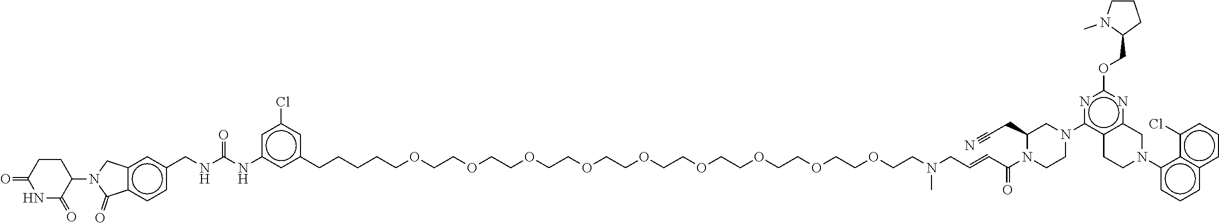 |
| I-163 | 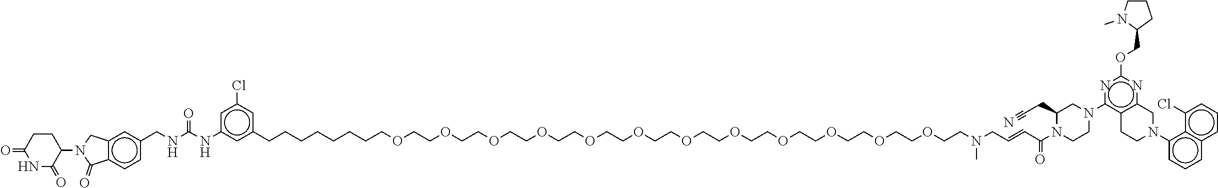 |
| I-164 | 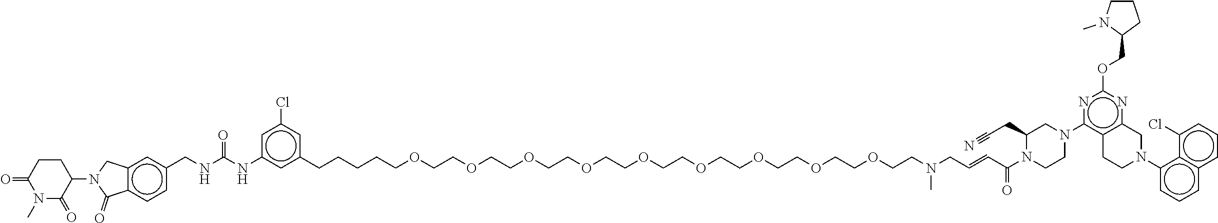 |
| I-165 | 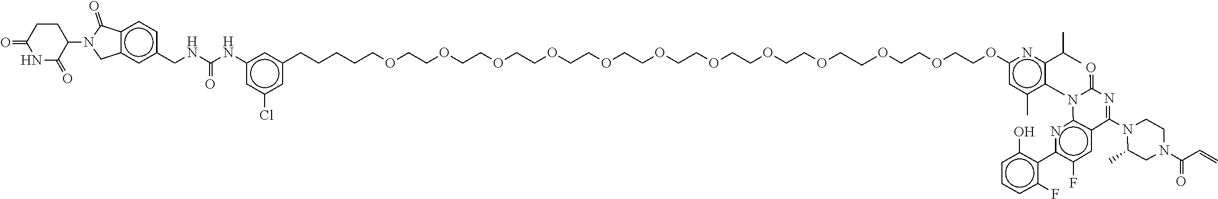 |

TABLE 2A

| | Target | MW | MH+ | | RT (wavelength- |
|---|---|---|---|---|---|
| Compd No. | Protein | Exact | (obs) | | min) |
| I-25 | ALK | 1232.4 | 617.5, | 1234.7 | 220-2.296 |
| I-26 | ALK | 1320.5 | 670.3 | | 220-2.292 |
| I-27 | ALK | 1304.5 | 653.5 | | 220-2.297 |
| I-28 | AR | 1202.5 | 602.9, | 1202.8 | 220-2.637 |
| I-29 | AR | 1290.5 | 645.9, | 646.9 | 220-2.633 |
| I-30 | BTK | 1202.5 | 602.4, | 1203.6 | 220-2.684 |
| I-31 | BTK | 1290.5 | 646.5 | | 220-2.693 |
| I-32 | BTK | 1378.6 | 690.4 | | 220-2.701 |
| I-33 | EGFR | 1217.5 | 609.5, | 1217.7 | 220-1.785 |
| I-34 | EGFR | 1201.5 | 601.5 | | 220-1.796 |
| I-35 | EGFR | 1289.6 | 645.4 | | 220-1.841 |
| I-36 | EGFR | 1377.6 | 689.9 | | 220-2.307 |
| I-37 | KRAS | 1569.7 | 784.7 | | 220-2.928 |
| I-38 | KRAS | 1477.7 | 740.6 | | 220-3.161 |
| I-39 | KRAS | 1565.7 | 782.7 | | 220-3.165 |
| I-40 | KRAS | 1301.6 | 651.9, | 1303.6 | 220-1.982 |
| I-41 | KRAS | 1389.6 | 696 | | 220-2.035 |
| I-42 | KRAS | 1585.7 | 793.9 | | 220-2.914 |
| I-43 | KRAS | 1391.6 | 697.5 | | 220-1.768 |
| I-44 | KRAS | 1479.6 | 741.9, | 1482.6 | 220-3.028 |
| I-45 | ALK | 1392.5 | 697.3 | | 220-2.318 |
| I-46 | KRAS | 1653.8 | 828.7 | | 220-3.153 |
| I-47 | KRAS | 1741.8 | 582.2, | 872.5 | 220-3.151 |
| I-48 | KRAS | 1297.6 | 650.4 | | 220-2.375 |
| I-49 | KRAS | 1385.7 | 694.9 | | 220-2.419 |
| I-50 | KRAS | 1473.7 | 738 | | 220-3.422 |
| I-51 | KRAS | 1561.8 | 783 | | 220-3.419 |
| I-52 | KRAS | 1649.8 | 551.5, | 826.6 | 220-3.423 |
| I-53 | KRAS | 1737.9 | 581.1, | 871.7 | 220-3.419 |
| I-54 | KRAS | 1303.5 | 653 | | 220-1.698 |
| I-55 | ER | 1233.5 | 617.9, | 1234.6 | 220-1.638 |
| I-56 | ER | 1317.6 | 659.9, | 1318.7 | 220-2.175 |
| I-57 | EGFR | 1305.6 | 654.0, | 1306.6 | 220-1.802 |
| I-58 | ER | 969.35 | 970.4 | | 220-2.186 |
| I-59 | ER | 1229.6 | 615.7, | 1230.7 | 220-2.189 |
| I-60 | EGFR | 1393.6 | 698 | | 220-1.879 |
| I-61 | EGFR | 1197.6 | 599.8, | 1198.6 | 220-2.163 |
| I-62 | EGFR | 1285.6 | 644.0, | 1286.6 | 220-2.172 |
| I-63 | EGFR | 1461.7 | 488.4, | 732.4 | 220-2.881 |
| I-64 | HER2 | 1259.6 | 631.0, | 1260.7 | 220-2.534 |
| I-65 | HER2 | 1347.7 | 675 | | 220-2.530 |
| I-66 | HER2 | 1523.8 | 509.1, | 763.0 | 220-2.869 |
| I-67 | BTK | 1195.6 | 598.9 | | 220-2.294 |
| I-68 | EGFR | 1373.7 | 688 | | 220-2.134 |
| I-69 | HER2 | 1435.7 | 718.9, | 1437.6 | 220-2.835 |
| I-70 | KRAS | 1315.6 | 658.8, | 659.8 | 220-1.961 |
| I-71 | KRAS | 1403.6 | 702.9, | 1405.4 | 220-2.023 |
| I-72 | BTK | 1283.6 | 643.0, | 1284.7 | 220-2.261 |
| I-73 | BTK | 1371.7 | 686.8 | | 220-2.276 |
| I-74 | BTK | 1226.6 | 614.4, | 1227.5 | 220-2.326 |
| I-75 | BTK | 1314.6 | 658.3 | | 220-2.356 |
| I-76 | BTK | 1402.7 | 702.4 | | 220-2.709 |
| I-77 | ER | 1145.5 | 574.3, | 1146.4 | 220-2.270 |
| I-78 | KRAS | 1491.7 | 746.9 | | 220-3.169 |
| I-80 | KRAS | 1579.7 | 791 | | 220-3.173 |
| I-81 | KRAS | 1340.6 | 671.5 | | 220-2.098 |
| I-82 | BTK | 1182.5 | 592.3, | 1183.5 | 220-2.010 |
| I-83 | BTK | 1459.7 | 487.9, | 730.9 | 220-2.646 |
| I-84 | KRAS | 1428.7 | 477.5, | 715.4 | 220-2.515 |
| I-85 | BTK | 1270.6 | 636.3, | 1271.4 | 220-2.520 |
| I-86 | KRAS | 1516.7 | 506.8, | 760.0 | 220-2.517 |
| I-87 | KRAS | 1604.8 | 536.1, | 803.9 | 220-2.532 |
| I-88 | KRAS | 1694.9 | 571.5, | 848.4 | 220-2.87 |
| I-89 | KRAS | 1316.9 | 658.9, | 1316.4 | 220-2.056 |
| I-90 | KRAS | 1405 | 1406.3 | | 220-2.48 |
| I-91 | KRAS | 1493.1 | 498.4, | 746.9 | 220-2.479 |
| I-92 | KRAS | 1581.2 | 527.8, | 790.9 | 220-2.487 |
| I-93 | BTK | 1360 | 680.4 | | 220-2.562 |
| I-94 | BTK | 1448.1 | 724.3 | | 220-2.876 |
| I-95 | KRAS | 1669.3 | 557.1, | 835.4 | 220-2.512 |
| I-96 | BTK | 1263.9 | 632.3 | | 220-2.178 |
| I-97 | BTK | 1352 | 676.3 | | 220-1.523 |
| I-98 | BTK | 1528.2 | 510.2, | 764.4 | 220-2.234 |
| I-99 | KRAS | 1298.9 | 649.8, | 1298.4 | 220-2.44 |
| I-100 | KRAS | 1783 | 600.8, | 892.5 | 220-2.558 |
| I-101 | BTK | 1440.1 | 720.5 | | 220-2.596 |
| I-102 | EGFR | 1112.7 | 556.9, | 1112.4 | 220-1.919 |
| I-103 | KRAS | 1727.9 | 576.8, | 865.0 | 220-2.803 |
| I-104 | BTK | 1198.8 | 599.9 | | 220-2.491 |
| I-105 | BTK | 1265.8 | 633.3, | 1265.5 | 220-1.978 |
| I-106 | KRAS | 1727.9 | 576.8, | 865.0 | 220-2.813 |
| I-107 | KRAS | 1569.6 | 785.5 | | 220-2.718 |
| I-108 | KRAS | 1344.4 | 672.4 | | 220-1.971 |
| I-109 | KRAS | 1446.6 | 724 | | 220-2.414 |
| I-110 | BTK | 1110.7 | 555.8, | 1110.4 | 220-2.464 |
| I-111 | BTK | 1286.9 | 643.9, | 1286.7 | 220-2.124 |
| I-112 | BTK | 1177.7 | 589.3 | | 220-2.368 |
| I-113 | BTK | 1353.9 | 677.5 | | 220-2.01 |
| I-114 | KRAS | 1358.5 | 680.3 | | 220-1.984 |
| I-115 | KRAS | 1270.3 | 635.9 | | 220-1.952 |
| I-116 | KRAS | 1358.5 | 680.3 | | 220-2.039 |
| I-117 | BTK | 1375 | 687.9 | | 220-2.138 |
| I-118 | KRAS | 1407.5 | 703.8 | | 220-2.392 |
| I-119 | ALK | 1216.2 | 608.3, | 608.9 | 220-2.024 |
| I-120 | ALK | 1304.3 | 652.3, | 653.0 | 220-2.457 |
| I-121 | ALK | 1128 | 565.2 | | 220-2.027 |
| I-122 | EGFR | 1028.5 | 514.7, | 1028.3 | 220-2.736 |
| I-123 | AR | 988.65 | 494.8, | 988.4 | 220-2.728 |
| I-124 | AR | 1076.8 | 538.9, | 1076.5 | 220-2.721 |
| I-125 | EGFR | 1200.8 | 600.9 | | 220-1.947 |
| I-126 | EGFR | 1292.8 | 646.9, | 1292.5 | 220-2.787 |
| I-127 | KRAS | 1495.6 | 748.5, | 1496.6 | 220-2.723 |
| I-128 | AR | 1164.9 | 582.9, | 1164.6 | 220-2.711 |
| I-129 | BTK | 1442 | 721.4 | | 220-2.444 |
| I-130 | EGFR | 1204.7 | 802.8, | 1204.5 | 220-2.458 |
| I-131 | EGFR | 1349.9 | 675.4 | | 220-2.323 |
| I-132 | EGFR | 1434.1 | 1435.7 | | 220-2.263 |
| I-133 | KRAS | 1319.3 | 659.9 | | 220-2.408 |
| I-134 | KRAS | 1495.6 | 747.9 | | 220-2.415 |
| I-135 | AR | 1253 | 627.4 | | 220-2.711 |
| I-136 | KRAS | 1494.5 | 747.8 | | 220-2.699 |
| I-137 | KRAS | 1407.5 | 704.8 | | 220-2.382 |
| I-138 | KRAS | 1583.7 | 792.8 | | 220-2.398 |
| I-139 | EGFR | 1288.9 | 644.8, | 1288.5 | 220-2.003 |
| I-140 | KRAS | 1319.3 | 659.8 | | 220-2.366 |
| I-141 | ALK | 1392.4 | 1391.3 | | 220-2.469 |
| I-142 | BTK | 1463.1 | 731.8 | | 220-2.919 |
| I-143 | BTK | 1530.2 | 765.4 | | 220-2.455 |
| I-144 | EGFR | 1261.8 | 607.8, | 1213.3 | 220-2.3 |
| I-145 | EGFR | 1169.8 | 1169.3 | | 220-1.657 |
| I-146 | EGFR | 1346 | 673.3 | | 220-1.732 |
| I-147 | KRAS | 1374 | 458.6, | 687.3 | 220-2.111 |
| I-148 | KRAS | 1550.2 | 517.3, | 775.4 | 220-2.792 |
| I-149 | KRAS | 1291.8 | 1291.3 | | 220-2.522 |
| I-150 | EGFR | 1085.6 | 543.2, | 1085.3 | 220-2.203 |
| I-151 | EGFR | 1257.9 | 629.3, | 1257.4 | 220-1.715 |
| I-152 | KRAS | 1285.9 | 643.3, | 1285.4 | 220-2.092 |
| I-153 | KRAS | 1379.9 | 690.3, | 1379.4 | 220-2.512 |
| I-154 | KRAS | 1468 | 734.3 | | 220-2.846 |
| I-155 | EGFR | 1116.6 | 558.7, | 1116.3 | 220-2.445 |
| I-156 | KRAS | 1462.1 | 488.0, | 731.3 | 220-2.476 |
| I-157 | KRAS | 1583.7 | 792.8 | | 220-2.728 |
| I-158 | KRAS | 1432.5 | 478.0, | 716.9 | 220-2.713 |
| I-159 | EGFR | 1173.7 | 1173.4, | 587.3 | 220-2.259 |
| I-160 | EGFR | 1377 | 690.3 | | 220-2.026 |
| I-161 | EGFR | 1465.1 | 732.9 | | 220-2.444 |
| I-162 | KRAS | 1520.6 | 507.6, | 761.0 | 220-2.424 |
| I-163 | KRAS | 1608.7 | 537.1, | 805.0 | 220-2.437 |
| I-164 | KRAS | 1534.7 | 512.5, | 768.4 | 220-2.476 |

| | | | | |
|---|---|---|---|---|
| I-165 | KRAS | 1556.1 | 778.4 | 220-2.838 |

### Example 26—GSPT1 HiBit Degradation Assay

Exemplary compounds were tested for ability to cause degradation of GSPT1. Experimental procedures and results are provided below.

### Part I—Experimental Procedure

Promega developed a HEK293 cell line that constitutively expresses LgBiT protein and GSPT1_HiBiT fusion protein. When both proteins are present, the HiBiT portion of the fusion protein combines with LgBiT protein to form a functional luciferase enzyme. Endurazine, a cell permeable small molecule, is converted by cellular esterases into furimazine, which the luciferase enzyme uses as a substrate to generate luminescence. When GSPT1 is degraded, the HiBit is also degraded. This prevents formation of fully functional luciferase, leading to a loss in luminescence. GSPT1 degradation therefore correlates with a decrease in luminescence and can be measured continuously over time. The concentration of compound at which 50% GSPT1 degradation occurs (DC₅₀) was calculated using this system.

### Reagents:

HEK293 LgBiT Cell line (Promega Custom)
Endurazine Substrate (Promega Cat. #N2570)
DMEM (Gibco Cat. #11995)
Penicillin/Streptomycin (Gibco Cat. #15140-122)
Heat Inactivated Fetal Bovine Serum (Gibco Cat. #A38400-01)
Hygromycin B (Gibco Cat. #10-687-010)
384 Well TC Treated White Microplate (Perkin Elmer Cat. #6007680)

### Protocol:

HEK293 LgBiT cells were cultured in DMEM Media supplemented with 10% FBS, 5% Pen/Strep, 200 μg/ml hygromycin and stored in an incubator set at 37° C. and 5% CO₂. 18-24 hours prior to compound treatment, HEK293 LgBiT cells were seeded at 1000 cells/well in a 384-well microplate in 25 μl complete media. The plate was spun at 300 g for 30 seconds and stored in the incubator overnight. The next day, compounds were titrated in DMSO and added to media supplemented with endurazine substrate. A 25 ul aliquot of 2× compound/endurazine in media were then added to the 384-well microplate with seeded cells. (Endurazine was used at a final concentration of 0.5×). The plate was spun at 300 g for 30 seconds and stored in the incubator. After 6 hours the plate was read on a Perkin Elmer MultiMode Plate Reader Envision 2105 to measure luminescence signal.

### Part II—Results

Experimental results showing GSPT1 degradation observed in the Hibit assay are provided in Table 3 and Table 3A, below. The symbol “++++” indicates a DC₅₀ less than 0.25 μM. The symbol “+++” indicates a DC₅₀ in the range of 0.25 μM to 5 μM. The symbol “++” indicates a DC₅₀ in the range of greater than 5 μM to 10 μM. The symbol “+” indicates a DC₅₀ of greater than 10 μM. The symbol “N/A” indicates that no data was available.

TABLE 3
GSPT1 Degradation (HiBit Assay)

| Compound | GSPT1 |
|---|---|
| No. | DC₅₀ |
| I-1 | +++ |
| I-2 | ++ |
| I-3 | + |
| I-4 | + |
| I-5 | ++++ |
| I-6 | + |
| I-7 | ++ |
| I-8 | + |
| I-9 | + |
| I-10 | ++ |
| I-11 | + |
| I-12 | ++ |
| I-13 | + |
| I-14 | + |
| I-15 | + |
| I-16 | + |
| I-17 | + |
| I-18 | + |
| I-19 | +++ |
| I-20 | +++ |
| I-21 | +++ |
| I-22 | ++++ |
| I-26 | + |
| I-27 | ++ |
| I-28 | +++ |
| I-29 | +++ |
| I-31 | +++ |
| I-33 | ++ |
| I-34 | ++ |
| I-35 | + |
| I-36 | ++ |
| I-37 | +++ |
| I-38 | ++++ |
| I-39 | ++++ |
| I-40 | ++++ |
| I-41 | ++++ |
| I-42 | + |
| I-43 | +++ |
| I-44 | +++ |
| I-45 | ++ |
| I-46 | ++++ |
| I-47 | ++++ |
| I-48 | +++ |
| I-49 | +++ |
| I-50 | +++ |
| I-51 | +++ |
| I-52 | +++ |
| I-53 | ++++ |
| I-54 | +++ |
| I-55 | ++++ |
| I-56 | + |
| I-57 | + |
| I-58 | ++++ |
| I-59 | +++ |
| I-60 | ++ |
| I-61 | ++++ |
| I-62 | ++++ |
| I-63 | ++++ |
| I-64 | ++++ |
| I-65 | ++++ |
| I-66 | +++ |
| I-67 | ++++ |
| I-68 | ++++ |
| I-69 | ++++ |
| I-70 | ++++ |
| I-71 | ++++ |
| I-72 | ++++ |
| I-73 | ++++ |
| I-74 | ++++ |
| I-75 | ++++ |
| I-76 | ++++ |

TABLE 3A
GSPT1 Degradation (HiBit Assay)

| Compound | GSPT1 |
|---|---|
| No. | DC₅₀ |
| I-77 | ++++ |
| I-78 | ++++ |
| I-80 | +++ |
| I-81 | ++++ |
| I-82 | ++++ |
| I-83 | +++ |
| I-84 | ++++ |
| I-85 | ++++ |
| I-86 | +++ |
| I-87 | +++ |
| I-88 | +++ |
| I-89 | ++++ |
| I-90 | ++++ |
| I-91 | +++ |
| I-92 | +++ |
| I-93 | ++++ |
| I-94 | +++ |
| I-95 | +++ |
| I-96 | ++++ |
| I-97 | ++++ |
| I-98 | ++++ |
| I-99 | +++ |
| I-100 | +++ |
| I-101 | ++++ |
| I-102 | +++ |
| I-103 | +++ |
| I-104 | +++ |
| I-105 | ++++ |
| I-106 | +++ |
| I-107 | +++ |
| I-108 | +++ |
| I-109 | ++ |
| I-110 | +++ |
| I-111 | +++ |
| I-112 | +++ |
| I-113 | +++ |
| I-114 | + |
| I-115 | ++ |
| I-116 | +++ |
| I-117 | ++ |
| I-118 | +++ |
| I-119 | +++ |
| I-120 | +++ |
| I-121 | ++++ |
| I-122 | ++++ |
| I-125 | +++ |
| I-126 | +++ |
| I-128 | ++++ |
| I-129 | +++ |
| I-130 | +++ |
| I-131 | +++ |
| I-132 | +++ |
| I-133 | +++ |
| I-134 | +++ |
| I-135 | +++ |
| I-136 | +++ |
| I-137 | +++ |
| I-138 | +++ |
| I-139 | +++ |
| I-140 | +++ |
| I-141 | +++ |
| I-142 | +++ |
| I-143 | +++ |
| I-145 | +++ |
| I-146 | +++ |
| I-147 | ++ |
| I-148 | +++ |
| I-149 | +++ |
| I-150 | +++ |
| I-151 | +++ |
| I-152 | + |
| I-153 | +++ |
| I-154 | +++ |
| I-155 | ++++ |
| I-156 | +++ |
| I-157 | +++ |
| I-159 | +++ |
| I-160 | +++ |
| I-161 | +++ |
| I-162 | +++ |
| I-163 | +++ |
| I-164 | ++ |
| I-165 | +++ |
| I-23 | + |
| I-24 | + |
| I-25 | + |

### Example 27—Cellular Growth Inhibition Assays

Exemplary compounds were tested for ability to inhibit the proliferation of HeLa KRas G12C cells, U937 cells, and/or 22RV1 cells. Experimental procedures and results are provided below.

### Part I—Experimental Procedure

Cells were seeded in Poly-D-lysine-treated 384-well white plates, with 25 μL cell suspension at 250 cells/well for continuous treatments, and 1000 cells/well for washout treatments. Plates were spun at 300×g for 3 minutes, and cells were cultured at 37° C. with 5% CO₂ in a humidified tissue culture incubator.

After 24 hours, the test compounds were diluted with cell culture medium in 96 deep well plates. DMSO was used as a negative control. A 25 μL aliquot of compound-containing medium was added in each well, at a final top concentration of 10 μM or 30 μM test compound, with 3-fold dilutions. For washout assays, after 4 hours, cell medium with compound was removed, and the cells were washed with 75p L fresh medium. Plates were then spun at 300×g for 3 minutes and replaced with 50 μL fresh medium in each well. Plates were then spun at 300×g for 3 minutes again and cultured at 37° C. with 5% CO₂.

At day-0 and day-5 of compound treatments, cell viability was quantified with CellTiter-Glo 2.0 (Promega). After equilibrating cell plates at room temperature for 30 minutes, 25 μL CellTiter-Glo 2.0 reagent was dispensed into each well (on top of 50 μL of cell culture). Plates were mixed on a shaker for 2 minutes at 500 rpm, followed by a 10-minute incubation at room temperature. Luminescence readings were measured with an EnVision Plate Reader. Data was normalized to DMSO treated day-0 cell wells. A four-parameter non-linear regression curve fit was applied to dose-response data in Prism to determine the half maximal growth inhibitory concentration (GI₅₀) of each compound.

### Part II—Results

Experimental results showing inhibition of HeLa KRas G12C cell growth observed in the cellular proliferation assay are provided in Table 4 below. The symbol “++++” indicates a GI₅₀ less than 1 μM. The symbol “+++” indicates a GI₅₀ in the range of 1 μM to 3 μM. The symbol “++” indicates a GI₅₀ in the range of greater than 3 μM to 10 μM. The symbol “+” indicates a GI₅₀ greater than 10 μM. The symbol “N/A” indicates that no data was available.

TABLE 4
Growth Inhibition of HeLa KRas G12C Cells (CTG Assay)

| Compound No. | GI₅₀ |
|---|---|
| I-3 | ++ |
| I-4 | ++ |
| I-6 | + |
| I-8 | + |
| I-5 | + |
| I-7 | + |
| I-13 | + |
| I-15 | + |
| I-14 | + |
| I-16 | + |
| I-17 | + |
| I-18 | + |
| I-1 | +++ |
| I-2 | + |
| I-9 | + |
| I-11 | + |
| I-10 | ++ |
| I-12 | ++ |
| I-42 | + |
| I-37 | + |
| I-54 | ++++ |
| I-43 | +++ |
| I-44 | +++ |
| I-40 | ++++ |
| I-41 | ++++ |
| I-38 | ++++ |
| I-39 | ++++ |
| I-46 | ++++ |
| I-47 | ++++ |
| I-48 | ++ |
| I-49 | + |
| I-50 | ++++ |
| I-51 | ++++ |
| I-52 | ++++ |
| I-53 | ++++ |
| I-116 | +++ |
| I-118 | +++ |
| I-134 | ++ |
| I-137 | ++ |
| I-138 | ++ |
| I-147 | ++ |
| I-148 | ++ |
| I-152 | ++ |

Experimental results showing inhibition of U937 cell growth observed in the cellular proliferation assay are provided in Table 5, below. The symbol “++++” indicates a GI₅₀ less than 0.1 μM. The symbol “+++” indicates a GI₅₀ in the range of 0.1 μM to 1 μM. The symbol “++” indicates a GI₅₀ in the range of greater than 1 μM to 5 μM. The symbol “+” indicates a GI₅₀ greater than 5 μM. The symbol “N/A” indicates that no data was available.

TABLE 5
Growth Inhibition of U937 Cells (CTG Assay)

| Compound No. | GI₅₀ |
|---|---|
| I-30 | +++ |
| I-31 | ++ |
| I-32 | ++ |
| I-67 | ++++ |
| I-72 | ++++ |
| I-73 | ++++ |
| I-74 | ++++ |
| I-75 | ++++ |
| I-76 | ++++ |

Experimental results showing inhibition of 22RV1 cell growth observed in the cellular proliferation assay are provided in Table 6 below. The symbol “++++” indicates a GI₅₀ less than 0.1 μM. The symbol “+++” indicates a GI₅₀ in the range of 0.1 μM to 1 μM. The symbol “++” indicates a GI₅₀ in the range of greater than 1 μM to 5 μM. The symbol “+” indicates a GI₅₀ greater than 5 μM. The symbol “N/A” indicates that no data was available.

TABLE 6
Growth Inhibition of 22RV1 Cells (CTG Assay)

| Compound No. | GI₅₀ |
|---|---|
| I-19 | ++++ |
| I-20 | ++++ |
| I-21 | +++ |
| I-22 | ++++ |
| I-28 | N/A |
| I-29 | N/A |

Experimental results showing growth inhibition of mutant EGFR T790M L858R cells in the cellular proliferation assay are provided in Table 7 below. The symbol “++++” indicates a GI₅₀ less than 0.1 μM. The symbol “+++” indicates a GI₅₀ in the range of 0.1 μM to 1 μM. The symbol “++” indicates a GI₅₀ in the range of greater than 1 μM to 5 μM. The symbol “+” indicates a GI₅₀ greater than 5 μM. The symbol “N/A” indicates that no data was available.

TABLE 7
Growth Inhibition of Mutant EGFR T790M L858R (CTG Assay)

| | CTG - 293 mutant |
|---|---|
| Compound No. | EGFR_T790M L858R Cells GI₅₀ |
| I-102 | ++ |
| I-120 | ++ |
| I-122 | +++ |
| I-125 | ++ |
| I-130 | +++ |
| I-131 | ++ |
| I-132 | ++ |
| I-144 | ++ |
| I-145 | ++ |
| I-146 | ++ |
| I-150 | ++ |
| I-151 | +++ |
| I-155 | +++ |
| I-159 | ++ |
| I-160 | +++ |
| I-161 | ++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 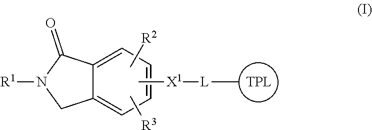
or a pharmaceutically acceptable salt thereof, wherein:
R¹ is 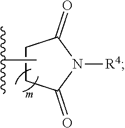
R² is hydrogen, halo, C₁₋₄ alkyl, or C₁₋₄haloalkyl;
R³ is hydrogen, halo, C₁₋₄ alkyl, C₁₋₄ haloalkyl, or —(C₁₋₄ alkylene)-N(R⁵)C(O)N(R⁵)(C₁₋₄ alkyl substituted with 1, 2, or 3 substituents independently selected from the group consisting of halo, hydroxyl, —N(R⁵)(R⁶), —N(R⁵)C(O)(R⁷), —C(O)N(R⁵)(R⁶), C₁₋₄ alkoxyl, phenyl, C₃₋₇ cycloalkyl, a 3-7 membered saturated heterocyclyl containing 1, 2, or 3 heteroatoms independently selected from oxygen, nitrogen, and sulfur, and a 5-12 membered heteroaryl containing 1, 2, or 3 heteroatoms independently selected from oxygen, nitrogen, and sulfur, wherein the phenyl, cycloalkyl, heterocyclyl, and heteroaryl are optionally substituted with 1, 2, or 3 occurrences of R);
R⁴ is hydrogen, C₁₋₄ alkyl, C₃₋₇ cycloalkyl, or —(C₁₋₄ alkylene)-C₃₋₇ cycloalkyl;
R⁵ and R⁶ each represent independently for each occurrence hydrogen, C₁₋₄ alkyl, C₃₋₇ cycloalkyl, or —(C₁₋₄ alkylene)-C₃₋₇ cycloalkyl; or an occurrence of R⁵ and R⁶ attached to same nitrogen atom are taken together with the nitrogen atom to which they are attached to form a 3-7 membered heterocyclic ring;
R⁷ represents independently for each occurrence C₁₋₄ alkyl, C₃₋₇ cycloalkyl, or —(C₁₋₄ alkylene)-C₃₋₇ cycloalkyl;
R⁸ represents independently for each occurrence halo, C₁₋₄ alkyl, C₁₋₄haloalkyl, hydroxyl, C₁₋₄ alkoxyl, —N(R⁵)(R⁶), —N(R⁵)C(O)(R⁷), or —C(O)N(R⁵)(R⁶);
R⁹ represents independently for each occurrence halo, C₁₋₄ alkyl, C₁₋₄ haloalkyl, hydroxyl, or C₁₋₄ alkoxyl; 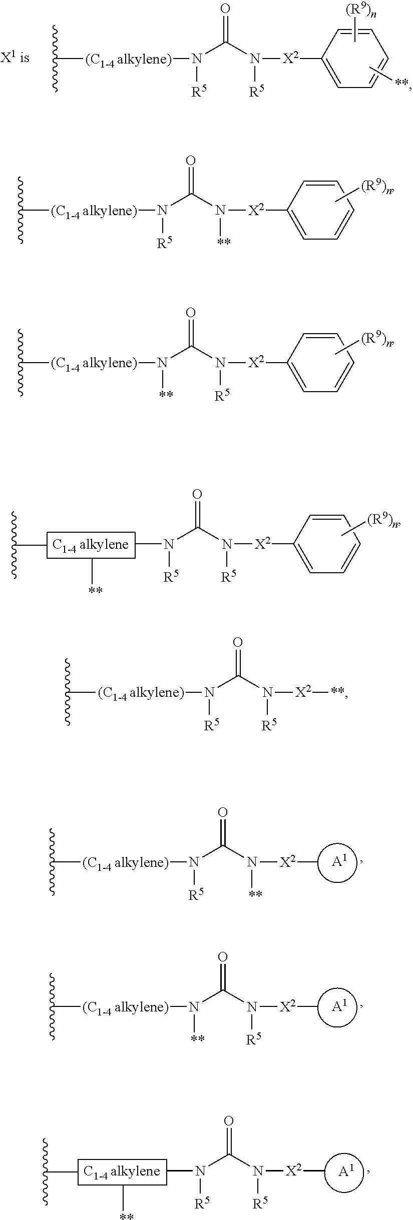
or a bond; wherein ** is a bond to L;
X² is a bond or a C₁₋₄ alkylene optionally substituted with 1, 2, or 3 occurrences of R⁸;
A¹ is a C₃₋₇ cycloalkyl, 3-7 membered saturated or partially saturated heterocyclyl containing 1, 2, or 3 heteroatoms independently selected from oxygen, nitrogen, and sulfur, or a 5-12 membered heteroaryl containing 1, 2, or 3 heteroatoms independently selected from oxygen, nitrogen, and sulfur, wherein each of the cycloalkyl, heterocyclyl, and heteroaryl is optionally substituted with 1, 2, or 3 occurrences of R⁹;
L is a linker;
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα;
m is 1, 2, or 3; and
n is 0, 1, 2, or 3.

2-6. (canceled)

7. The compound of claim 1, wherein the compound is represented by Formula I-A: 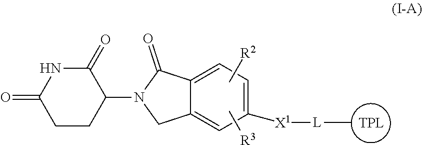
or a pharmaceutically acceptable salt thereof, wherein:
R² is hydrogen, halo, or C₁₋₄ alkyl;
R³ is hydrogen, halo, C₁₋₄ alkyl, or —N(R⁵)(R⁶);
R⁵ and R⁶ each represent independently for each occurrence hydrogen or C₁₋₄ alkyl;
R⁹ represents independently for each occurrence halo, C₁₋₄ alkyl, C₁₋₄ haloalkyl, hydroxyl, or C₁₋₄ alkoxyl;
X¹ is 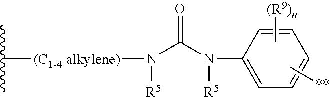
wherein ** is a bond to L;
L is a linker;
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα; and
n is 0, 1, 2, or 3.

8. (canceled)

9. The compound of claim 7, wherein X¹ is

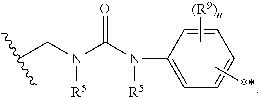

10. (canceled)

11. (canceled)

12. The compound of claim 7, wherein X¹ is

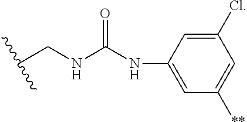

13. (canceled)

14. (canceled)

15. (canceled)

16. A compound represented by Formula II: 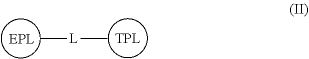
or a pharmaceutically acceptable salt thereof, wherein:
EPL is a moiety that binds to an effector protein selected from GSPT1, Cyclin K, RBM23, RBM39, IKZF1, IKZF3, a PLK1 degrader protein, a CDK4 degrader protein, or CK1alpha;
L is a linker; and
TPL is a moiety that binds to a target protein selected from KRAS, HER2, BTK, EGFR, androgen receptor protein, estrogen receptor protein, ALK, IDH1, FLT3, FGFR1, FGFR4, HCV-NS3, FGFR2, FGFR3, ERK1, ERK2, FGR, HER3, HER4, or PI3Kα.

17-30. (canceled)

31. The compound of claim 1, wherein the TPL is a moiety that binds to KRAS.

32. The compound of claim 1, wherein the TPL is one of the following: 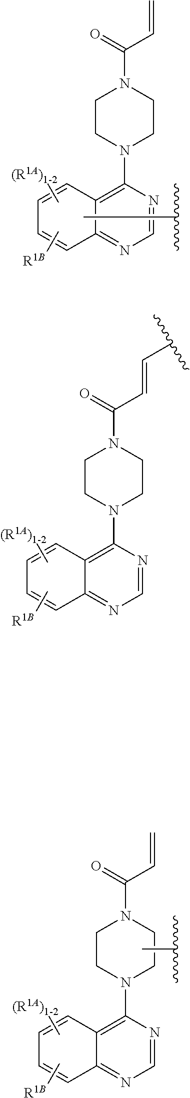
wherein:
R^{1A} represents independently for each occurrence hydrogen, halo, hydroxyl, C₁₋₄ alkyl, or C₁₋₄ alkoxyl; and
R^{1B} is C₆₋₁₂ aryl optionally substituted by 1, 2, or 3 substituents independently selected from halo, hydroxyl, C₁₋₄ alkyl, or C₁₋₄ alkoxyl.

33. The compound of claim 1, wherein the TPL is one of the following: 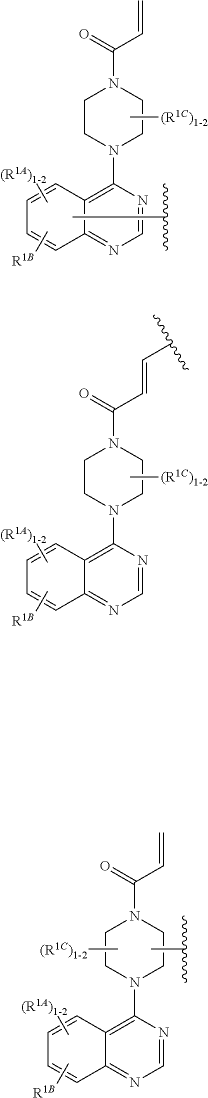 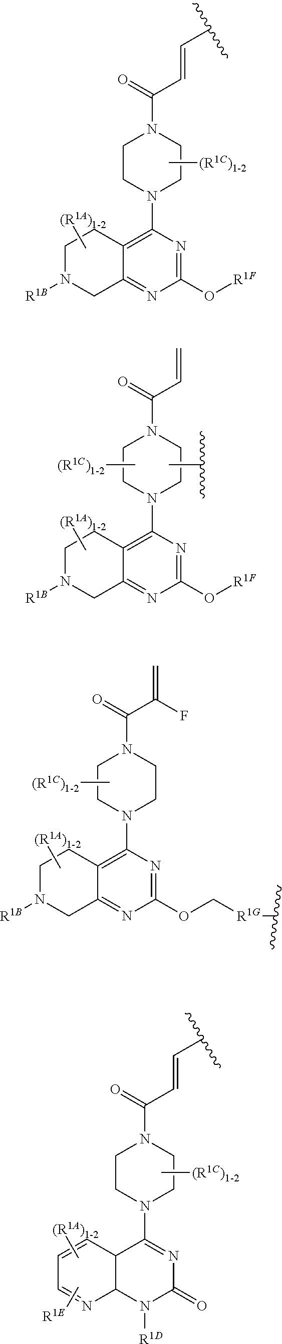 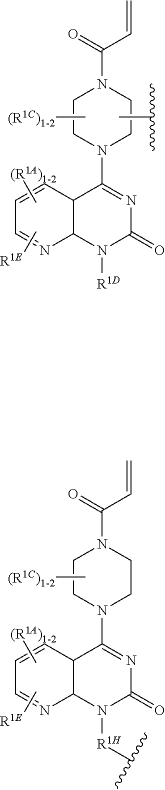
wherein:
R^{1A} represents independently for each occurrence hydrogen, halo, hydroxyl, C₁₋₄ alkyl, or C₁₋₄ alkoxyl;
R^{1B} is C₆₋₁₂ aryl optionally substituted by 1, 2, or 3 substituents independently selected from halo, hydroxyl, C₁₋₄ alkyl, or C₁₋₄ alkoxyl;
R^{1C} represents independently for each occurrence (C₁₋₄ alkylene)-CN, or C₁₋₄ alkyl;
R^{1D} are R^{1E} are independently phenyl or 5-6 membered heteroaryl containing 1, 2, or 3 heteroatoms selected from oxygen, nitrogen, or sulfur, wherein the phenyl or heteroaryl is substituted with 0, 1, 2, or 3 substituents independently selected from the group consisting of halo, hydroxyl, C₁₋₄ alkyl, or C₁₋₄ alkoxyl;
R^{1F} is —(C₁₋₄ alkylene)-(3-7 membered heterocyclyl containing 1, 2, or 3 heteroatoms independently selected from oxygen, nitrogen, or sulfur, wherein the heterocyclyl is substituted with 0, 1, 2, or 3 substituents independently selected from the group consisting of halo, hydroxyl, C₁₋₄ alkyl, or C₁₋₄ alkoxyl);
R^{1G} is 3-7 membered heterocyclylene containing 1, 2, or 3 heteroatoms independently selected from heteroatoms independently selected from oxygen, nitrogen, or sulfur, wherein the heterocyclylene is substituted with 0, 1, 2, or 3 substituents independently selected from the group consisting of halo, hydroxyl, C₁₋₄ alkyl, or C₁₋₄ alkoxyl); and
R^{1H} is phenylene or 5-6 membered heteroarylene containing 1, 2, or 3 heteroatoms selected from oxygen, nitrogen, or sulfur, wherein the phenylene or heteroarylene is substituted with 0, 1, 2, or 3 substituents independently selected from the group consisting of halo, hydroxyl, C₁₋₄ alkyl, or C₁₋₄ alkoxyl.

34. The compound of claim 1, wherein the TPL is one of the following:

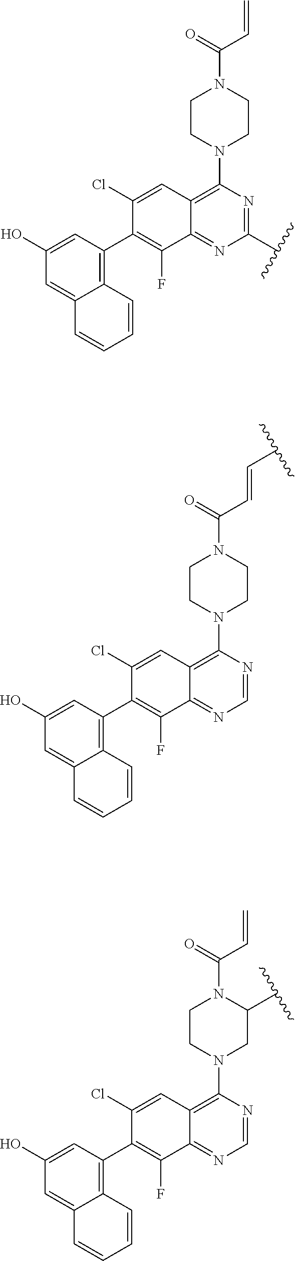 [SMILES: *c1nc(N2CCN(C(=O)C=C)CC2)c2cc(Cl)c(-c3cc(O)cc4ccccc34)c(F)c2n1 ; */C=C/C(=O)N1CCN(c2ncnc3c(F)c(-c4cc(O)cc5ccccc45)c(Cl)cc23)CC1 ; *C1CN(c2ncnc3c(F)c(-c4cc(O)cc5ccccc45)c(Cl)cc23)CCN1C(=O)C=C]

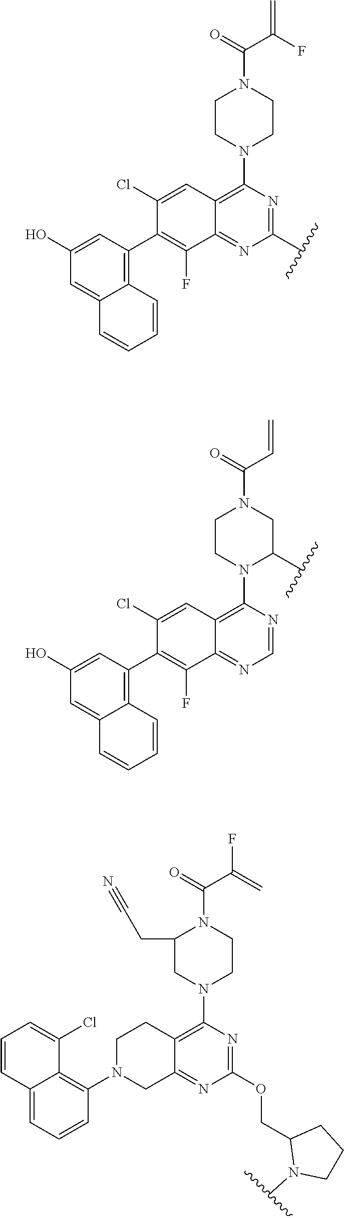 [SMILES: *c1nc(N2CCN(C(=O)C(=C)F)CC2)c2cc(Cl)c(-c3cc(O)cc4ccccc34)c(F)c2n1 ; *C1CN(C(=O)C=C)CCN1c1ncnc2c(F)c(-c3cc(O)cc4ccccc34)c(Cl)cc12 ; *N1CCCC1COc1nc2c(c(N3CCN(C(=O)C(=C)F)C(CC#N)C3)n1)CCN(c1cccc3cccc(Cl)c13)C2]

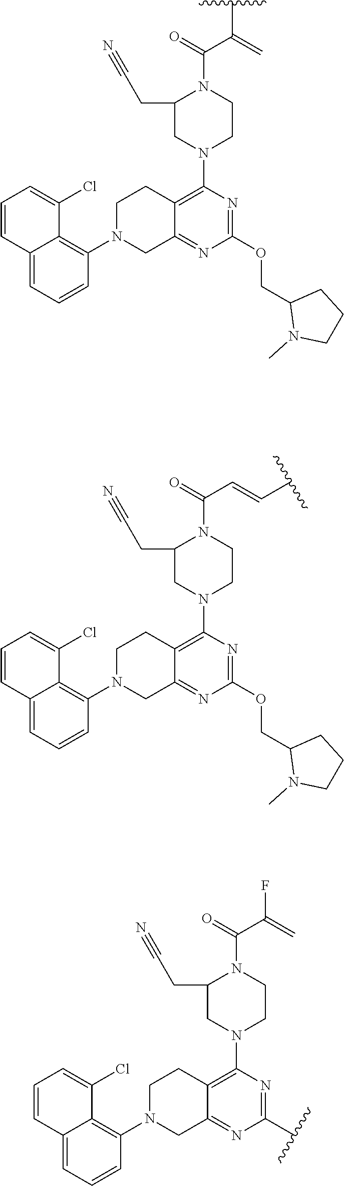 [SMILES: *C(=C)C(=O)N1CCN(c2nc(OCC3CCCN3C)nc3c2CCN(c2cccc4cccc(Cl)c24)C3)CC1CC#N ; */C=C/C(=O)N1CCN(c2nc(OCC3CCCN3C)nc3c2CCN(c2cccc4cccc(Cl)c24)C3)CC1CC#N ; *c1nc2c(c(N3CCN(C(=O)C(=C)F)C(CC#N)C3)n1)CCN(c1cccc3cccc(Cl)c13)C2]

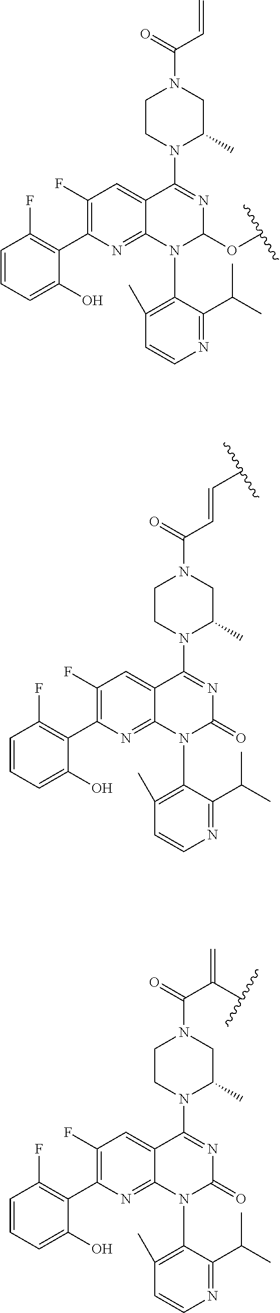 [SMILES: *OC1N=C(N2CCN(C(=O)C=C)C[C@@H]2C)c2cc(F)c(-c3c(O)cccc3F)nc2N1c1c(C)ccnc1C(C)C ; */C=C/C(=O)N1CCN(c2nc(=O)n(-c3c(C)ccnc3C(C)C)c3nc(-c4c(O)cccc4F)c(F)cc23)[C@@H](C)C1 ; *C(=C)C(=O)N1CCN(c2nc(=O)n(-c3c(C)ccnc3C(C)C)c3nc(-c4c(O)cccc4F)c(F)cc23)[C@@H](C)C1]

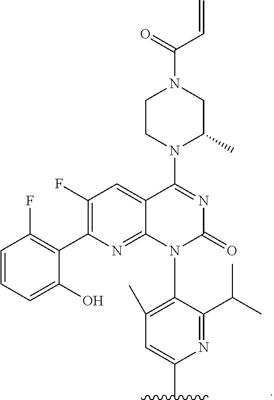 [SMILES: *c1cc(C)c(-n2c(=O)nc(N3CCN(C(=O)C=C)C[C@@H]3C)c3cc(F)c(-c4c(O)cccc4F)nc32)c(C(C)C)n1]

35. (canceled)

36. The compound of claim 1, wherein the TPL is a moiety that binds to HER2.

37. The compound of claim 1, wherein the TPL is one of the following 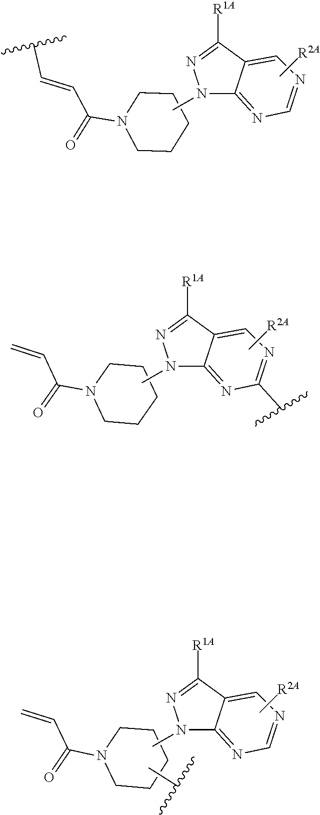
wherein:
R^{1A} is —C(O)(NR^{5A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R^{5A})(R^{6A}));
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

38. The compound of claim 1, wherein the TPL is one of the following:

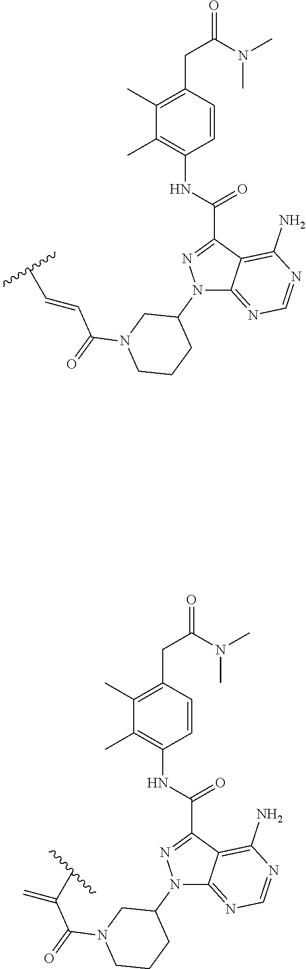 [SMILES: */C=C/C(=O)N1CCCC(n2nc(C(=O)Nc3ccc(CC(=O)N(C)C)c(C)c3C)c3c(N)ncnc32)C1 ; *C(=C)C(=O)N1CCCC(n2nc(C(=O)Nc3ccc(CC(=O)N(C)C)c(C)c3C)c3c(N)ncnc32)C1]

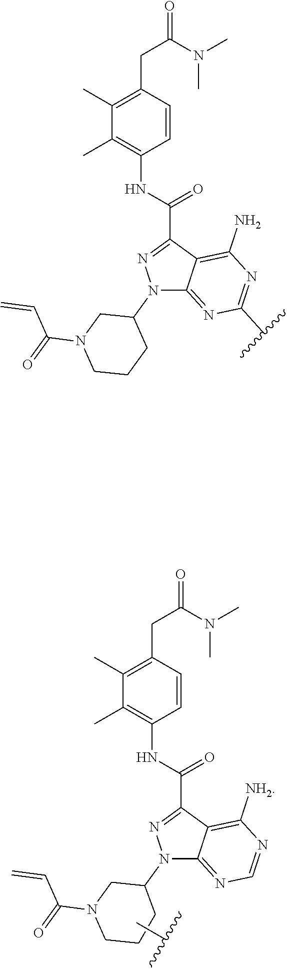

39. The compound of claim 1, wherein the TPL is a moiety that binds to BTK.

40. The compound of claim 1, wherein the TPL is one of the following: 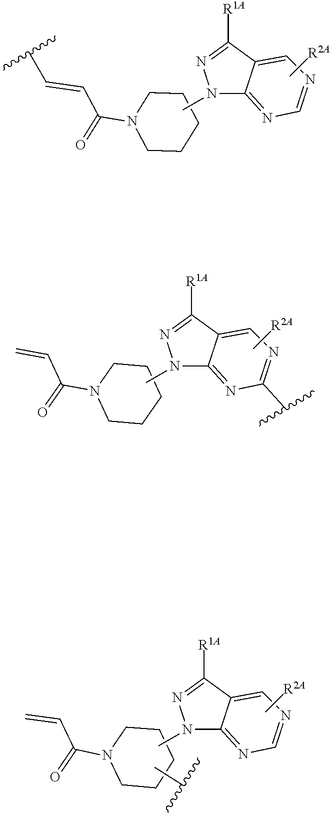
wherein:
R^{1A} is -(phenyl optionally substituted with 1, 2, 3, or 5 substituents independently selected from halo, hydroxyl, C₁₋₄ alkyl, and C₁₋₄ alkoxyl)-O-(phenyl optionally substituted with 1, 2, 3, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

41. The compound of claim 1, wherein the TPL is: 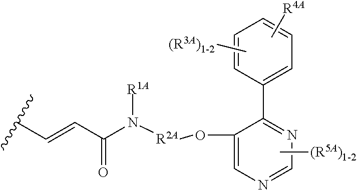
wherein:
R^{1A}, R^{6A} and R^{7A} are independently hydrogen or C₁₋₄ alkyl;
R^{2A} is C₁₋₄ alkylene;
R^{3A} and R^{5A} each represent independently for each occurrence hydrogen, halo, hydroxyl, C₁₋₄ alkyl, C₁₋₄ alkoxyl, or —N(R^{8A})(R^{9A});
R^{4A} is —N(R^{6A})C(O)R^{7A};
R^{7A} is phenyl or 5-6 membered heteroaryl containing 1, 2, or 3 heteroatoms selected from oxygen, nitrogen, or sulfur, wherein the phentyl and heteroaryl are substituted with 0, 1, 2, or 3 substituents independently selected from the group consisting of halo, hydroxyl, C₁₋₄ alkyl, C₃₋₇ cycloalkyl, C₁₋₄ alkoxyl, and —N(R^{8A})(R^{9A});
R^{8A} and R^{9A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{8A} and R^{9A} attached to same nitrogen atom are taken together with the nitrogen atom to which they are attached to form a 3-7 membered heterocyclic ring.

42. The compound of claim 1, wherein the TPL is one of the following:

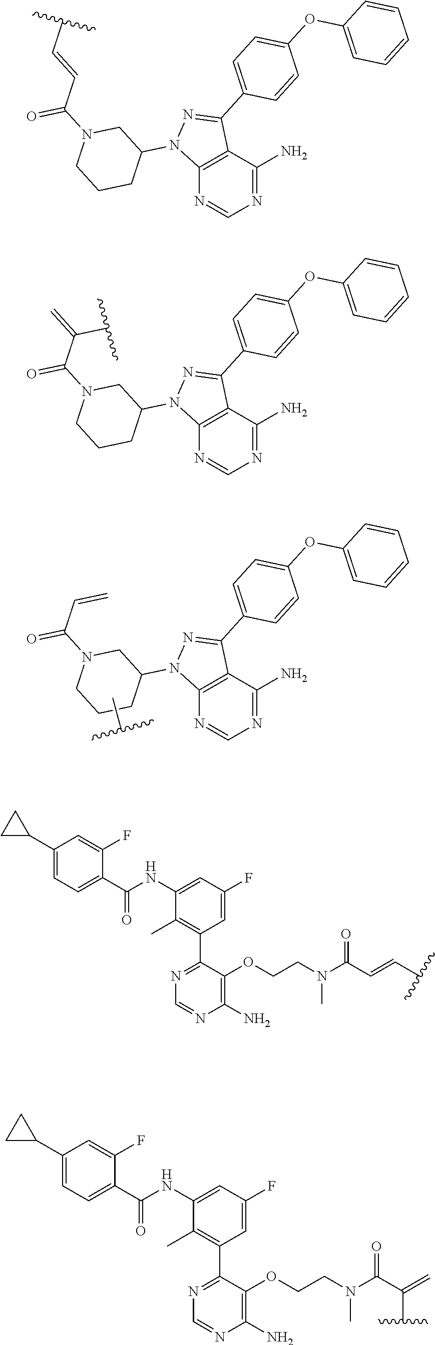

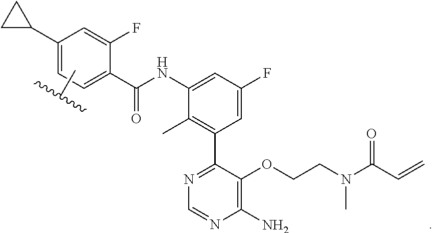

43. The compound of claim 1, wherein the TPL is a moiety that binds to EGFR.

44. The compound of claim 1, wherein the TPL is one of the following: 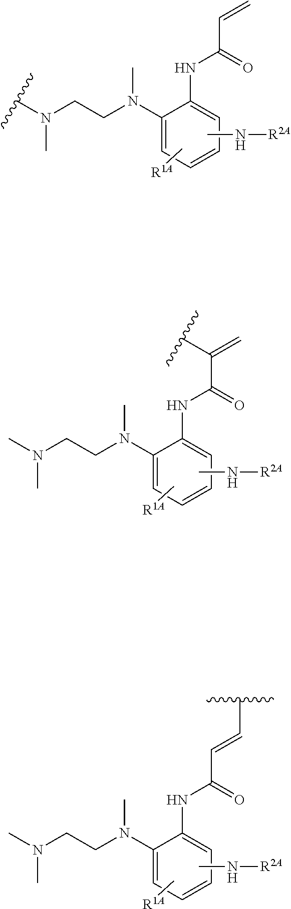
wherein:
R^{1A} is hydrogen, halo, hydroxyl, C₁₋₄ alkyl, C₁₋₄ alkoxyl, or N(R^{5A})(R^{6A}); and
R^{2A} is -(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

45. The compound of claim 1, wherein the TPL is one of the following: 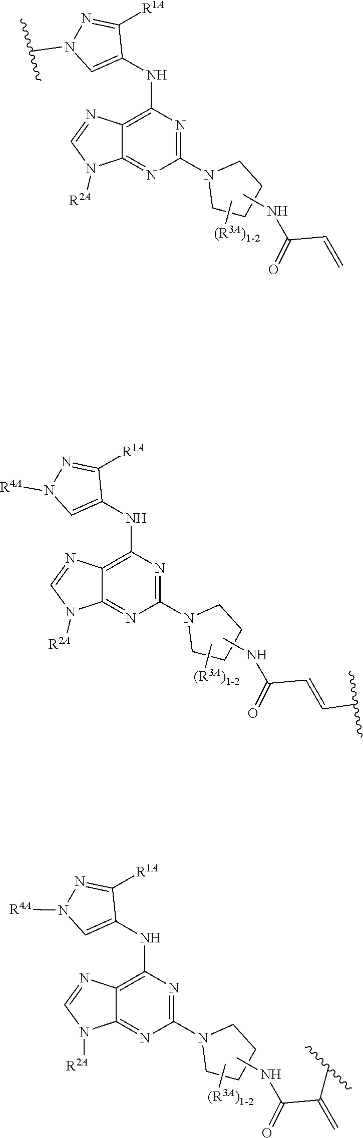
wherein:
R^{1A} is C₁₋₄ alkoxyl, C₁₋₄ alkyl, hydrogen, halo, or hydroxyl;
R^{2A} and R^{4A} are independently hydrogen or C₁₋₄ alkyl; anH
R^{3A} represents independently for each occurrence hydrogen, halo, hydroxyl, C₁₋₄ alkyl, or C₁₋₄ alkoxyl.

46. (canceled)

47. The compound of claim 1, wherein the TPL is a moiety that binds to androgen receptor protein.

48. The compound of claim 1, wherein the TPL is one of the following: 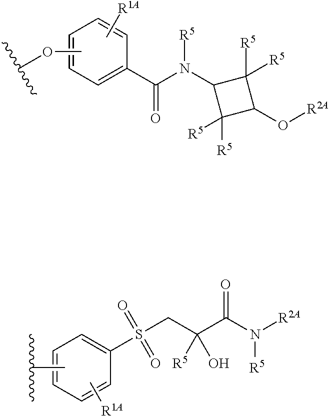
wherein:
R^{1A} is hydrogen, halo, hydroxyl, C₁₋₄ alkyl, C₁₋₄ alkoxyl, or —N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

49. The compound of claim 1, wherein the TPL is one following:

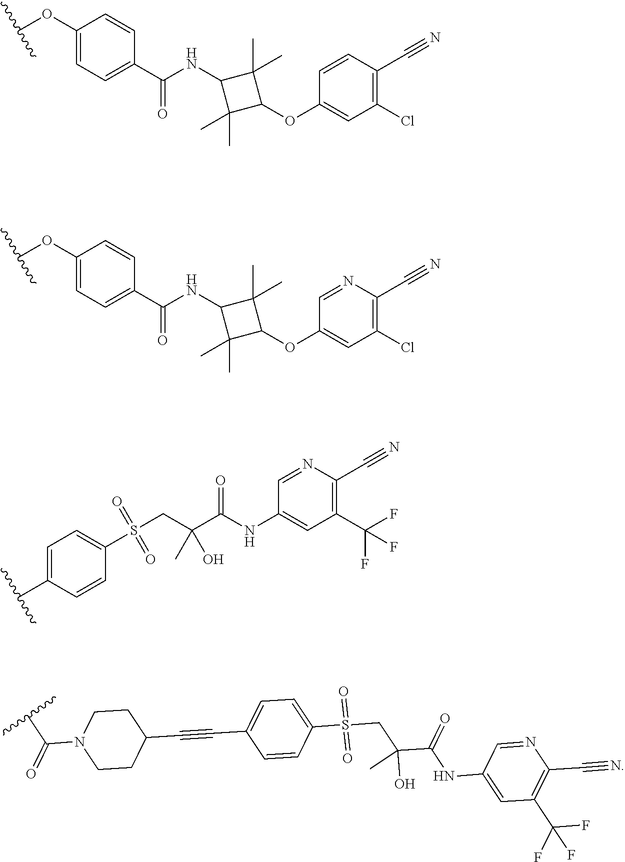 [SMILES: *Oc1ccc(C(=O)NC2C(C)(C)C(Oc3ccc(C#N)c(Cl)c3)C2(C)C)cc1 ; *Oc1ccc(C(=O)NC2C(C)(C)C(Oc3cnc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1]

50-56. (canceled)

57. The compound of claim 1, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

58. (canceled)

59. (canceled)

60. (canceled)

61. (canceled)

62. The compound of claim 1, wherein L is —CH₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄—***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)₁₁—***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where *** is a point of attachment to TPL.

63. The compound of claim 1, wherein L is —(C₂₋₂₀ alkylene)-(OCH₂CH₂)₂₋₄—(C₀₋₄ alkylene)—***, —(C₂₋₂₀alkylene)-(OCH₂CH₂)₅₋₇—(C₀₋₄ alkylene)—***, —(C₂₋₂₀ alkylene)-(OCH₂CH₂)₈₋₁₀—(C₀₋₄ alkylene)—***, —(C₂₋₂₀alkylene)-(OCH₂CH₂)₁₁₋₁₃—(C₀₋₄ alkylene)—***, —(C₂₋₂₀alkylene)-(OCH₂CH₂)₁₄₋₁₆—(C₀₋₄ alkylene)—***, —(C₂₋₂₀alkylene)-(OCH₂CH₂)₁₇₋₂₀—(C₀₋₄ alkylene)—***, —(C₁₋₂₀ alkylene)-(OCH₂CH₂)₁₋₁₀—(C₀₋₄ alkylene)-C(O)—*** or —(C₁₋₂₀ alkylene)-(OCH₂CH₂)₁₁₋₂₀—(C₀₋₄ alkylene)-C(O)—***, where *** is a point of attachment to TPL.

64-66. (canceled)

67. A compound in Table 3 or 3A, or a pharmaceutically acceptable salt thereof.

68. (canceled)

69. A pharmaceutical composition comprising a compound of claim 1 and a pharmaceutically acceptable carrier.

70. A method of treating cancer, comprising administering to a patient in need thereof a therapeutically effective amount of a compound of claim 1 to treat the cancer.

71. The method of claim 70, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

72-76. (canceled)
